SEQUENCE LISTING (537 sequences)

SEQ ID NO: 1 - protein, 9 aa, synthetic construct
VVGACGVGK

SEQ ID NO: 2 - protein, 10 aa, synthetic construct
VVVGACGVGK

SEQ ID NO: 3 - protein, 278 aa, synthetic construct
MASGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 4 - protein, 278 aa, synthetic construct
MASGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 5 - protein, 225 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWIAAMDYWGQGTLVTVFNQIKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 6 - protein, 216 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRKTITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 10 aa, synthetic construct
KLVVVGACGV

SEQ ID NO: 8 - protein, 9 aa, synthetic construct
QLMPFGCLL

SEQ ID NO: 9 - protein, 10 aa, synthetic construct
LMPFGCLLDY

SEQ ID NO: 10 - protein, 9 aa, synthetic construct
MPFGCLLDY

SEQ ID NO: 11 - protein, 10 aa, synthetic construct
YMANGCLLNY

SEQ ID NO: 12 - protein, 17 aa, synthetic construct
SISSSSGSTSYADSVKG

SEQ ID NO: 13 - protein, 9 aa, synthetic construct
  VARIANT 2: note = G, R, H, S, or K
  VARIANT 4: note = Y or I
  VARIANT 5: note = P or A
GXWXXAMDY

SEQ ID NO: 14 - protein, 11 aa, synthetic construct
RASQSVSSAVA

SEQ ID NO: 15 - protein, 7 aa, synthetic construct
SASSLYS

SEQ ID NO: 16 - protein, 11 aa, synthetic construct
  VARIANT 3: note = I, A, P, V, or S
  VARIANT 7: note = K, R, A, or H
  VARIANT 8: note = K or R
  VARIANT 9: note = L, T, K, R, V, A, or E
QQXSYVXXXIT

SEQ ID NO: 17 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRKTITFGQGTKVEIKRTV

SEQ ID NO: 18 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWIAAMDYWGQGTLVTVSS

SEQ ID NO: 19 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRRTITFGQGTKVEIKRTV

SEQ ID NO: 20 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQPSYVRRKITFGQGTKVEIKRTV

SEQ ID NO: 21 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGRWIPAMDYWGQGTLVTVSS

SEQ ID NO: 22 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVARKITFGQGTKVEIKRTV

SEQ ID NO: 23 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQVSYVARRITFGQGTKVEIKRTV

SEQ ID NO: 24 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVKRLITFGQGTKVEIKRTV

SEQ ID NO: 25 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGHWIAAMDYWGQGTLVTVSS

SEQ ID NO: 26 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRRLITFGQGTKVEIKRTV

SEQ ID NO: 27 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRRVITFGQGTKVEIKRTV

SEQ ID NO: 28 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGSWIAAMDYWGQGTLVTVSS

SEQ ID NO: 29 - protein, 111 aa, synthetic construct
DIQMTQSPSSLSASVGDRVTITCRASQSVSSAVAWYQQKPGKAPKLLIYSASSLYSGVPSRFSGSRSGTDTLTISSLQPEDFATYYCQQASYVAKTITFGQGTKVEIKRTV

SEQ ID NO: 30 - protein, 112 aa, synthetic construct
DIQMTQSPSSLSASVGDRVTITCRAGQSVSSAVAWYQQKPGKAPKLLIYSASSLYSGVPSRFSGSRSGTDFTLTISSLQPEDFATYYCQQSSYVRRKITFGQGTKVEIKRTV

SEQ ID NO: 31 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGKWIPAMDYWGQGTLVTVSS

SEQ ID NO: 32 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRKAITFGQGTKVEIKRTV

SEQ ID NO: 33 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRRAITFGQGTKVEIKRTV

SEQ ID NO: 34 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSYVKRTITFGQGTKVEIKRTV

SEQ ID NO: 35 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQPSYVRKTITFGQGTKVEIKRTV

SEQ ID NO: 36 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVKKEITFGQGTKVEIKRTV

SEQ ID NO: 37 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSYVHKLITFGQGTKVEIKRTV

SEQ ID NO: 38 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRREITFGQGTKVEIKRTV

SEQ ID NO: 39 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVHRLITFGQGTKVEIKRTV

SEQ ID NO: 40 - protein, 5 aa, synthetic construct
SSYIH

SEQ ID NO: 41 - protein, 17 aa, synthetic construct
YISPSYGSTSYADSVKG

SEQ ID NO: 42 - protein, 9 aa, synthetic construct
  VARIANT 2: note = Y, L, S, or E
  VARIANT 3: note = V, T, or I
  VARIANT 4: note = T or I
  VARIANT 6: note = A, T, or S
  VARIANT 7: note = L, A, I, K, P, or T
EXXXMXXDY

SEQ ID NO: 43 - protein, 9 aa, synthetic construct
  VARIANT 4: note = S or A, or S
  VARIANT 5: note = Y, H, A, D, E, K, S, or G
  VARIANT 8: note = I or E
QQYXXWPXT

SEQ ID NO: 44 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSHWPITFGQGTKVEIKRTV

SEQ ID NO: 45 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELVTMTADYWGQGTLVTVSS

SEQ ID NO: 46 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSAWPETFGQGTKVEIKRTV

SEQ ID NO: 47 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYTTMSIDYWGQGTLVTVSS

SEQ ID NO: 48 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSAWPITFGQGTKVEIKRTV

SEQ ID NO: 49 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARESVTMSADYWGQGTLVTVSS

SEQ ID NO: 50 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSDWPITFGQGTKVEIKRTV

SEQ ID NO: 51 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARESVTMTKDYWGQGTLVTVSS

SEQ ID NO: 52 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYAEWPITFGQGTKVEIKRTV

SEQ ID NO: 53 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREEVTMSIDYWGQGTLVTVSS

SEQ ID NO: 54 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARESITMTKDYWGQGTLVTVSS

SEQ ID NO: 55 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYAKWPITFGQGTKVEIKRTV

SEQ ID NO: 56 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELTTMSIDYWGQGTLVTVSS

SEQ ID NO: 57 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSSWPITFGQGTKVEIKRTV

SEQ ID NO: 58 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELTEMTTDYWGQGTLVTVSS

SEQ ID NO: 59 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREEVTMTADYWGQGTLVTVSS

SEQ ID NO: 60 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSGWPITFGQGTKVEIKRTV

SEQ ID NO: 61 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELIEMTPDYWGQGTLVTVSS

SEQ ID NO: 62 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELVTMTIDYWGQGTLVTVSS

SEQ ID NO: 63 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYASWPITFGQGTKVEIKRTV

SEQ ID NO: 64 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELTTMTADYWGQGTLVTVSS

SEQ ID NO: 65 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARESITMSPDYWGQGTLVTVSS

SEQ ID NO: 66 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELIEMTTDYWGQGTLVTVSS

SEQ ID NO: 67 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMSIDYWGQGTLVTVSS

SEQ ID NO: 68 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELVEMTPDYWGQGTLVTVSS

SEQ ID NO: 69 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMALDYWGQGTLVTVSS

SEQ ID NO: 70 - protein, 118 aa, synthetic construct
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREEVTMTPDYWGQGTLVTVSS

SEQ ID NO: 71 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSHWPETFGQGTKVEIKRTV

SEQ ID NO: 72 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSEWPETFGQGTKVEIKRTV

SEQ ID NO: 73 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSS

SEQ ID NO: 74 - protein, 107 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 75 - protein, 512 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQYSYWPITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMALDYWGQGTLVTVSSLEGGGGLNDIFEAQKIEWHESRHHHHHH

SEQ ID NO: 76 - protein, 512 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQYSDWPITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMSIDYWGQGTLVTVSSLEGGGGLNDIFEAQKIEWHESRHHHHHH

SEQ ID NO: 77 - protein, 512 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQYSSWPITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSYIHWVRQAPGKGLEWVAYISPSYGSTSYADSVKGRFTISADTSKNTAYLQMNSLRAEDTAVYYCARELVEMTPDYWGQGTLVTVSSLEGGGGLNDIFEAQKIEWHESRHHHHHH

SEQ ID NO: 78 - protein, 28 aa, synthetic construct
LEGGGGLNDIFEAQKIEWHESRHHHHHH

SEQ ID NO: 79 - protein, 514 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQASYVRRTITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWIAAMDYWGQGTLVTVSSLEGGGGLNDIFEAQKIEWHESRHHHHHH

SEQ ID NO: 80 - protein, 514 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQASYVRRVITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWIAAMDYWGQGTLVTVSSLEGGGGLNDIFEAQKIEWHESRHHHHHH

SEQ ID NO: 81 - protein, 514 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQASYVRKTITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWIAAMDYWGQGTLVTVSSLEGGGGLNDIFEAQKIEWHESRHHHHHH

SEQ ID NO: 82 - protein, 9 aa, synthetic construct
MFPGCLLDY

SEQ ID NO: 83 - protein, 9 aa, synthetic construct
GGWIAAMDY

SEQ ID NO: 84 - protein, 11 aa, synthetic construct
QQASYVRKTIT

SEQ ID NO: 85 - protein, 9 aa, synthetic construct
GSWIHAMDY

SEQ ID NO: 86 - protein, 17 aa, synthetic construct
SISSSWGVTSYADSVKG

SEQ ID NO: 87 - protein, 7 aa, synthetic construct
FHWYSIH

SEQ ID NO: 88 - protein, 118 aa, synthetic construct
EVQLVESGGGLVQPGGSLRLSCAASGFTFHWYSIHWVRQAPGKGLEWVASISSSWGVTSYADSVKGRFTISADTSKNTAYLQMNSLRAEDTAVYYCARGSWIHAMDYWGQGTLVTVSS

SEQ ID NO: 89 - protein, 9 aa, synthetic construct
GHWIAAMDY

SEQ ID NO: 90 - protein, 17 aa, synthetic construct
SIASSSGSTGYADSVKG

SEQ ID NO: 91 - protein, 7 aa, synthetic construct
FSWYSIH

SEQ ID NO: 92 - protein, 118 aa, synthetic construct
EVQLVESGGGLVQPGGSLRLSCAASGFTFSWYSIHWVRQAPGKGLEWVASIASSSGSTGYADSVKGRFTISADTSKNTAYLQMNSLRAEDTAVYYCARGHWIAAMDYWGQGTLVTVSS

SEQ ID NO: 93 - protein, 9 aa, synthetic construct
GGVIHAMDY

SEQ ID NO: 94 - protein, 17 aa, synthetic construct
SILSRWGVTSYADSVKG

SEQ ID NO: 95 - protein, 7 aa, synthetic construct
FSPYSIH

SEQ ID NO: 96 - protein, 118 aa, synthetic construct
EVQLVESGGGLVQPGGSLRLSCAASGFTFSPYSIHWVRQAPGKGLEWVASILSRWGVTSYADSVKGRFTISADTSKNTAYLQMNSLRAEDTAVYYCARGGVIHAMDYWGQGTLVTVSS

SEQ ID NO: 97 - protein, 9 aa, synthetic construct
GSWIAAMDY

SEQ ID NO: 98 - protein, 17 aa, synthetic construct
SISSWHGETGYADSVKG

SEQ ID NO: 99 - protein, 118 aa, synthetic construct
EVQLVESGGGLVQPGGSLRLSCAASGFTFSPYSIHWVRQAPGKGLEWVASISSWHGETGYADSVKGRFTISADTSKNTAYLQMNSLRAEDTAVYYCARGSWIAAMDYWGQGTLVTVSS

SEQ ID NO: 100 - protein, 17 aa, synthetic construct
SISSLQGDTGYADSVKG

SEQ ID NO: 101 - protein, 118 aa, synthetic construct
EVQLVESGGGLVQPGGSLRLSCAASGFTFSWYSIHWVRQAPGKGLEWVASISSLQGDTGYADSVKGRFTISADTSKNTAYLQMNSLRAEDTAVYYCARGGWIAAMDYWGQGTLVTVSS

SEQ ID NO: 102 - protein, 17 aa, synthetic construct
SIASWYGDTGYADSVKG

SEQ ID NO: 103 - protein, 7 aa, synthetic construct
FHYYSIH

SEQ ID NO: 104 - protein, 118 aa, synthetic construct
EVQLVESGGGLVQPGGSLRLSCAASGFTFHYYSIHWVRQAPGKGLEWVASIASWYGDTGYADSVKGRFTISADTSKNTAYLQMNSLRAEDTAVYYCARGSWIAAMDYWGQGTLVTVSS

SEQ ID NO: 105 - protein, 7 aa, synthetic construct
FGYYSIH

SEQ ID NO: 106 - protein, 9 aa, synthetic construct
GGRIEAMDY

SEQ ID NO: 107 - protein, 17 aa, synthetic construct
SISSWYGKTGYADSVKG

SEQ ID NO: 108 - protein, 118 aa, synthetic construct
EVQLVESGGGLVQPGGSLRLSCAASGFTFGYYSIHWVRQAPGKGLEWVASISSWYGKTGYADSVKGRFTISADTSKNTAYLQMNSLRAEDTAVYYCARGGRIEAMDYWGQGTLVTVSS

SEQ ID NO: 109 - protein, 9 aa, synthetic construct
GYWIEAMDY

SEQ ID NO: 110 - protein, 17 aa, synthetic construct
SIASSYGSTGYADSVKG

SEQ ID NO: 111 - protein, 7 aa, synthetic construct
FSKYSIH

SEQ ID NO: 112 - protein, 118 aa, synthetic construct
EVQLVESGGGLVQPGGSLRLSCAASGFTFSKYSIHWVRQAPGKGLEWVASIASSYGSTGYADSVKGRFTISADTSKNTAYLQMNSLRAEDTAVYYCARGYWIEAMDYWGQGTLVTVSS

SEQ ID NO: 113 - protein, 17 aa, synthetic construct
SIHSSIGTTGYADSVKG

SEQ ID NO: 114 - protein, 7 aa, synthetic construct
FGLYSIH

SEQ ID NO: 115 - protein, 118 aa, synthetic construct
EVQLVESGGGLVQPGGSLRLSCAASGFTFGLYSIHWVRQAPGKGLEWVASIHSSIGTTGYADSVKGRFTISADTSKNTAYLQMNSLRAEDTAVYYCARGSWIAAMDYWGQGTLVTVSS

SEQ ID NO: 116 - protein, 9 aa, synthetic construct
GSVIHAMDY

SEQ ID NO: 117 - protein, 17 aa, synthetic construct
SILSWIGKTSYADSVKG

SEQ ID NO: 118 - protein, 118 aa, synthetic construct
EVQLVESGGGLVQPGGSLRLSCAASGFTFSPYSIHWVRQAPGKGLEWVASILSWIGKTSYADSVKGRFTISADTSKNTAYLQMNSLRAEDTAVYYCARGSVIHAMDYWGQGTLVTVSS

SEQ ID NO: 119 - protein, 17 aa, synthetic construct
SIASRWGHTGYADSVKG

SEQ ID NO: 120 - protein, 7 aa, synthetic construct
FSPYHIH

SEQ ID NO: 121 - protein, 118 aa, synthetic construct
EVQLVESGGGLVQPGGSLRLSCAASGFTFSPYHIHWVRQAPGKGLEWVASIASRWGHTGYADSVKGRFTISADTSKNTAYLQMNSLRAEDTAVYYCARGGWIAAMDYWGQGTLVTVSS

SEQ ID NO: 122 - protein, 17 aa, synthetic construct
SIASLQGITGYADSVKG

SEQ ID NO: 123 - protein, 7 aa, synthetic construct
FHEYSIH

SEQ ID NO: 124 - protein, 118 aa, synthetic construct
EVQLVESGGGLVQPGGSLRLSCAASGFTFHEYSIHWVRQAPGKGLEWVASIASLQGITGYADSVKGRFTISADTSKNTAYLQMNSLRAEDTAVYYCARGSWIAAMDYWGQGTLVTVSS

SEQ ID NO: 125 - protein, 7 aa, synthetic construct
FSDYSIH

SEQ ID NO: 126 - protein, 118 aa, synthetic construct
EVQLVESGGGLVQPGGSLRLSCAASGFTFSDYSIHWVRQAPGKGLEWVASILSRWGVTSYADSVKGRFTISADTSKNTAYLQMNSLRAEDTAVYYCARGGVIHAMDYWGQGTLVTVSS

SEQ ID NO: 127 - protein, 17 aa, synthetic construct
SISSRWGVTSYADSVKG

SEQ ID NO: 128 - protein, 118 aa, synthetic construct
EVQLVESGGGLVQPGGSLRLSCAASGFTFSDYSIHWVRQAPGKGLEWVASISSRWGVTSYADSVKGRFTISADTSKNTAYLQMNSLRAEDTAVYYCARGGVIHAMDYWGQGTLVTVSS

SEQ ID NO: 129 - protein, 10 aa, synthetic construct
VVVGADGVGK

SEQ ID NO: 130 - protein, 9 aa, synthetic construct
VVGADGVGK

SEQ ID NO: 131 - protein, 10 aa, synthetic construct
KLVVVGADGV

SEQ ID NO: 132 - protein, 10 aa, synthetic construct
VVVGASGVGK

SEQ ID NO: 133 - protein, 9 aa, synthetic construct
VVGASGVGK

SEQ ID NO: 134 - protein, 10 aa, synthetic construct
KLVVVGASGV

SEQ ID NO: 135 - protein, 10 aa, synthetic construct
VVVGARGVGK

SEQ ID NO: 136 - protein, 9 aa, synthetic construct
VVGARGVGK

SEQ ID NO: 137 - protein, 10 aa, synthetic construct
KLVVVGARGV

SEQ ID NO: 138 - protein, 9 aa, synthetic construct
  SITE 5: note = Residue is conjugated to Sotorasib (AMG-510)
VVGACGVGK

SEQ ID NO: 139 - protein, 10 aa, synthetic construct
  SITE 6: note = Residue is conjugated to Sotorasib (AMG-510)
VVVGACGVGK

SEQ ID NO: 140 - protein, 9 aa, synthetic construct
VVGAGGVGK

SEQ ID NO: 141 - protein, 10 aa, synthetic construct
VVVGAGGVGK

SEQ ID NO: 142 - protein, 10 aa, synthetic construct
  SITE 8: note = Residue is conjugated to Sotorasib (AMG-510)
KLVVVGACGV

SEQ ID NO: 143 - protein, 5 aa, synthetic construct
DYSIH

SEQ ID NO: 144 - protein, 9 aa, synthetic construct
GGWYPAMDY

SEQ ID NO: 145 - protein, 9 aa, synthetic construct
GRWIPAMDY

SEQ ID NO: 146 - protein, 9 aa, synthetic construct
GKWIPAMDY

SEQ ID NO: 147 - protein, 11 aa, synthetic construct
QQISYVKKLIT

SEQ ID NO: 148 - protein, 11 aa, synthetic construct
QQASYVRRTIT

SEQ ID NO: 149 - protein, 11 aa, synthetic construct
QQPSYVRRKIT

SEQ ID NO: 150 - protein, 11 aa, synthetic construct
QQASYVARKIT

SEQ ID NO: 151 - protein, 11 aa, synthetic construct
QQVSYVARRIT

SEQ ID NO: 152 - protein, 11 aa, synthetic construct
QQASYVKRLIT

SEQ ID NO: 153 - protein, 11 aa, synthetic construct
QQASYVRRLIT

SEQ ID NO: 154 - protein, 11 aa, synthetic construct
QQASYVRRVIT

SEQ ID NO: 155 - protein, 11 aa, synthetic construct
QQASYVAKTIT

SEQ ID NO: 156 - protein, 11 aa, synthetic construct
QQSSYVRRKIT

SEQ ID NO: 157 - protein, 11 aa, synthetic construct
QQASYVRKAIT

SEQ ID NO: 158 - protein, 11 aa, synthetic construct
QQASYVRRAIT

SEQ ID NO: 159 - protein, 11 aa, synthetic construct
QQSSYVKRTIT

SEQ ID NO: 160 - protein, 11 aa, synthetic construct
QQPSYVRKTIT

SEQ ID NO: 161 - protein, 11 aa, synthetic construct
QQASYVKKEIT

SEQ ID NO: 162 - protein, 11 aa, synthetic construct
QQSSYVHKLIT

SEQ ID NO: 163 - protein, 11 aa, synthetic construct
QQASYVRREIT

SEQ ID NO: 164 - protein, 11 aa, synthetic construct
QQASYVHRLIT

SEQ ID NO: 165 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKRTV

SEQ ID NO: 166 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSS

SEQ ID NO: 167 - protein, 9 aa, synthetic construct
EYVTMALDY

SEQ ID NO: 168 - protein, 9 aa, synthetic construct
ELVTMTADY

SEQ ID NO: 169 - protein, 9 aa, synthetic construct
EYTTMSIDY

SEQ ID NO: 170 - protein, 9 aa, synthetic construct
ESVTMSADY

SEQ ID NO: 171 - protein, 9 aa, synthetic construct
ESVTMTKDY

SEQ ID NO: 172 - protein, 9 aa, synthetic construct
EEVTMSIDY

SEQ ID NO: 173 - protein, 9 aa, synthetic construct
ESITMTKDY

SEQ ID NO: 174 - protein, 9 aa, synthetic construct
ELTTMSIDY

SEQ ID NO: 175 - protein, 9 aa, synthetic construct
ELTEMTTDY

SEQ ID NO: 176 - protein, 9 aa, synthetic construct
EEVTMTADY

SEQ ID NO: 177 - protein, 9 aa, synthetic construct
ELIEMTPDY

SEQ ID NO: 178 - protein, 9 aa, synthetic construct
ELVTMTIDY

SEQ ID NO: 179 - protein, 9 aa, synthetic construct
ELTTMTADY

SEQ ID NO: 180 - protein, 9 aa, synthetic construct
ESITMSPDY

SEQ ID NO: 181 - protein, 9 aa, synthetic construct
ELIEMTTDY

SEQ ID NO: 182 - protein, 9 aa, synthetic construct
EYVTMSIDY

SEQ ID NO: 183 - protein, 9 aa, synthetic construct
ELVEMTPDY

SEQ ID NO: 184 - protein, 9 aa, synthetic construct
EEVTMTPDY

SEQ ID NO: 185 - protein, 9 aa, synthetic construct
QQYSYWPIT

SEQ ID NO: 186 - protein, 9 aa, synthetic construct
QQYSHWPIT

SEQ ID NO: 187 - protein, 9 aa, synthetic construct
QQYSAWPET

SEQ ID NO: 188 - protein, 9 aa, synthetic construct
QQYSAWPIT

SEQ ID NO: 189 - protein, 9 aa, synthetic construct
QQYSDWPIT

SEQ ID NO: 190 - protein, 9 aa, synthetic construct
QQYAEWPIT

SEQ ID NO: 191 - protein, 9 aa, synthetic construct
QQYAKWPIT

SEQ ID NO: 192 - protein, 9 aa, synthetic construct
QQYSSWPIT

SEQ ID NO: 193 - protein, 9 aa, synthetic construct
QQYSGWPIT

SEQ ID NO: 194 - protein, 9 aa, synthetic construct
QQYASWPIT

SEQ ID NO: 195 - protein, 9 aa, synthetic construct
QQYSHWPET

SEQ ID NO: 196 - protein, 9 aa, synthetic construct
QQYSEWPET

SEQ ID NO: 197 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSYWPITFGQGTKVEIKRTV

SEQ ID NO: 198 - protein, 4 aa, synthetic construct
GGGS

SEQ ID NO: 199 - protein, 20 aa, synthetic construct
GGGGSGGGGSGGGGSGGGGS

SEQ ID NO: 200 - protein, 15 aa, synthetic construct
GGGGSGGGGSGGGGS

SEQ ID NO: 201 - protein, 9 aa, synthetic construct
GHWTPAMDY

SEQ ID NO: 202 - protein, 20 aa, synthetic construct
  VARIANT 1..20: note = This sequence may encompass 2-4 GGGGS repeating units
GGGGSGGGGSGGGGSGGGGS

SEQ ID NO: 203 - protein, 15 aa, synthetic construct
  VARIANT 1..15: note = This sequence may encompass 1-3 GGGGS repeating units
GGGGSGGGGSGGGGS

SEQ ID NO: 204 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 205 - protein, 50 aa, synthetic construct
  VARIANT 1..50: note = This sequence may encompass 1-10 SSSSG repeating units
SSSSGSSSSGSSSSGSSSSGSSSSGSSSSGSSSSGSSSSGSSSSGSSSSG

SEQ ID NO: 206 - protein, 8 aa, synthetic construct
GGGGSAAA

SEQ ID NO: 207 - protein, 11 aa, synthetic construct
QQASYVRRVVT

SEQ ID NO: 208 - protein, 9 aa, synthetic construct
GGWKPAMDY

SEQ ID NO: 209 - protein, 18 aa, synthetic construct
EGRGSLLTCGDVEENPGP

SEQ ID NO: 210 - protein, 17 aa, synthetic construct
ATNFSLKQAGDVENPGP

SEQ ID NO: 211 - protein, 19 aa, synthetic construct
QCTNYALKLAGDVESNPGP

SEQ ID NO: 212 - protein, 21 aa, synthetic construct
VKQTLNFDLKLAGDVESNPGP

SEQ ID NO: 213 - protein, 6 aa, synthetic construct
HHHHHH

SEQ ID NO: 214 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQDWYFPITFGQGTKVEIK

SEQ ID NO: 215 - protein, 128 aa, synthetic construct
EVQLVESGGGLVQPGGSLRLSCAASGFTFSSYYIHWVRQAPGKGLEWVASISPSSGSTYYADSVKGRFTISADTSKNTAYLQMNSLRAEDTAVYYCARYGGRSYWQKQDSYFYQHGLDYWGQGTLVSS

SEQ ID NO: 216 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 217 - protein, 125 aa, synthetic construct
EVQLVESGGGLVQPGGSLRLSCAASGFTFSSSSIHWVRQAPGKGLEWVASISSYSGYTSYADSVKGRFTISADTSKNTAYLQMNSLRAEDTAVYYCARSYSYSEFRYYYSGQGMDYWGQGTLVSS

SEQ ID NO: 218 - protein, 123 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSNYGWRWHLVGMDYWGQGTLVTVSS

SEQ ID NO: 219 - protein, 123 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSPYVYYWYMVGFDYWGQGTLVTVSS

SEQ ID NO: 220 - protein, 15 aa, synthetic construct
  SITE 15: note = Residue attached to Tetraethylene glycol monoamine lysine-long chain biotin-amide
YKLVVVGACGVGKSA

SEQ ID NO: 221 - protein, 11 aa, synthetic construct
  SITE 11: note = Lysine side chain attached to long chain Biotin-amide
SSSSCSSSSWK

SEQ ID NO: 222 - protein, 110 aa, synthetic construct
QSVLIQPRSVSGSPGQSVTISCTGTSSDVGGYNYVSWYQQHPGKAPKLMIYDVSKRPSGVPDRFSGSKSGNTASLTVSGLQAEDEADYYCGSYADTDTIVFGTGTKLTVL

SEQ ID NO: 223 - protein, 119 aa, synthetic construct
QVQLVQSEPEVKKPGSSVKLSCKASGGTFSTDAITWVRQAPGQGLEYMGGIIPLLDSVDYAQRFQGRVTVSADKSTGTAYMEVRSLGSEDTAKYYCAKWSSVDTGLDYWGQGTLVTVSS

SEQ ID NO: 224 - protein, 126 aa, synthetic construct
QVQLQESGPGLVKPSETLSLTCTVSGDSIINDPHYWGWIRQSPGKGLEWIGSTSHSGHTYFNPSLKSRVSMSIDVAKNQFSLNVRSVTAADTAVYYCARMRYYYSGTYPVYYFDYWGQGTLVTVSS

SEQ ID NO: 225 - protein, 110 aa, synthetic construct
SYVLTQPPSASGTPGQRVTISCSGSSSNIGSNFVSWYQQLPGTAPKLLISSNNQRPSGVPDRFSGSKSDTSASLAISGLQSEDEADYYCAAWDDSLNGPVFGGGTQLTVL

SEQ ID NO: 226 - protein, 121 aa, synthetic construct
QVQLVQSEAEVKKPGSSVKVSCKASGGTFSRYGVSWVRQAPGQGLEWMGGIIPMFGTANYAQKFQGRVTITADESTSTAYMELRSLRSEDTAVYYCARGDNSAYSDAFNIWGQGTMVTVSS

SEQ ID NO: 227 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGWSYPITFGQGTKVEIKRTV

SEQ ID NO: 228 - protein, 117 aa, synthetic construct
EVQLVESGGGLVQPGGSLRLSCAASGFTFYSSYIHWVRQAPGKGLEWVASISPYYGSTSYADSVKGRFTISADTSKNTAYLQMNSLRAEDTAVYYCARSSYYALDYWGQGTLVTVSS

SEQ ID NO: 229 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVYSLITFGQGTKVEIKRTV

SEQ ID NO: 230 - protein, 118 aa, synthetic construct
EVQLVESGGGLVQPGGSLRLSCAASGFTFSSYSIHWVRQAPGKGLEWVASIYSSYGYTSYADSVKGRFTISADTSKNTAYLQMNSLRAEDTAVYYCARGGWYPAMDYWGQGTLVTVSS

SEQ ID NO: 231 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDLATYYCQQYQYGYNLITFGQGTKVEIKRTV

SEQ ID NO: 232 - protein, 124 aa, synthetic construct
EVQLVESGGGLVQPGGSLRLSCAASGFTISYSSIHWVRQAPGKGLEWVASIYSYSGSTSYADSVKGRFTISADTSKNTAYLQMNSLRAEDTAVYYCARYSYGWVGPGWRAIDYWGQGTLVTVSS

SEQ ID NO: 233 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSSVYKLLTFGQGTKVEIKRTV

SEQ ID NO: 234 - protein, 120 aa, synthetic construct
EVQLVESGGGLVQPGGSLRLSCAASGFTVYYSSIHWVRQAPGKGLEWVASISSSYSYTSYADSVKGRFTISADTSKNTAYLQMNSLRAEDTALYYCARGGPGWYRAMDYWGQGTLVTVSS

SEQ ID NO: 235 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTV

SEQ ID NO: 236 - protein, 123 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARGYFYYGWWAMAFDYWGQGTLVTVSS

SEQ ID NO: 237 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSQWYEPLITFGQGTKVEIKRTV

SEQ ID NO: 238 - protein, 127 aa, synthetic construct
EVQLVESGGGLVQPGGSLRLSCAASGFTIYSSYIHWVRQAPGKGLEWVASISSSSGSTSYADSVKGRFTISADTSKNTAYLQMNSLRAEDTAVYYCARSYSYMSQWGWYQYSGMDYWGQGTLVTVSS

SEQ ID NO: 239 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGSYTYRLITFGQGTKVEIKRTV

SEQ ID NO: 240 - protein, 119 aa, synthetic construct
EVQLVESGGGLVQPGGSLRLSCAASGFTVSYSSIHWVRQAPGKGLEWVASISSSSGYTSYADSVKGRFTISADTSKNTAYLQMNSLRAEDTAVYYCARYAWWAHGLDYWGQGTLVTVSS

SEQ ID NO: 241 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYWYNLFTFGQGTKVEIKRTV

SEQ ID NO: 242 - protein, 123 aa, synthetic construct
EVQLVESGGGLVQPGGSLRLSCAASGFTISSYSIHWVRQAPGKGLEWVASIYSSYGYTSYADSVKGRFTISADTSKNTAYLQMNSLRAEDTAVYYCARQYSMHFPWGYGMDYWGQGTLVTVSS

SEQ ID NO: 243 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSDMPPITFGQGTKVEIKRTV

SEQ ID NO: 244 - protein, 121 aa, synthetic construct
EVQLVESGGGLVQPGGSLRLSCAASGFTFYSSSIHWVRQAPGKGLEWVAYIYSSSGYTSYADSVKGRFTISADTSKNTAYLQMNSLRAEDTAVYYCARPVNYYYQGALDYWGQGTLVTVSS

SEQ ID NO: 245 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYVFPITFGQGTKVEIKRTV

SEQ ID NO: 246 - protein, 130 aa, synthetic construct
EVQLVESGGGLVQPGGSLRLSCAASGFTVYSSSIHWVRQAPGKGLEWVASISPSSGYTYYADSVKGRFTISADTSKNTAYLQMNSLRAEDTAVYYCARYHYMFEYDKGESKWGYYGFDYWGQGTLVTVSS

SEQ ID NO: 247 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSQYFPITFGQGTKVEIKRTV

SEQ ID NO: 248 - protein, 122 aa, synthetic construct
EVQLVESGGGLVQPGGSLRLSCAASGFTISYSSIHWVRQAPGKGLEWVASIYSYYGYTSYADSVKGRFTISADTSKNTAYLQMNSLRAEDTAVYYCARNSWSWYSGVGMDYWGQGTLVTVSS

SEQ ID NO: 249 - protein, 123 aa, synthetic construct
EVQLVESGGGLVQPGGSLRLSCAASGFTISSSSIHWVRQAPGKGLEWVASISSYSSSTYYADSVKGRFTISADTSKNTAYLQMNSLRAEDTAVYYCARYPYGWGWGGSGLDYWGQGTLVTVSS

SEQ ID NO: 250 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQFDFQYLITFGQGTKVEIKRTV

SEQ ID NO: 251 - protein, 117 aa, synthetic construct
EVQLVESGGGLVQPGGSLRLSCAASGFTVYYSSIHWVRQAPGKGLEWVASIYSYYGSTYYADSVKGRFTISADTSKNTAYLQMNSLRAEDTAVYYCARGEKWALDYWGQGTLVTVSS

SEQ ID NO: 252 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYMYYQPLITFGQGTKVEIKRTV

SEQ ID NO: 253 - protein, 121 aa, synthetic construct
EVQLVESGGGLVQPGGSLRLSCAASGFTVYYSSIHWVRQAPGKGLEWVASISSSSGSTSYADSVKGRFTISADTSKNTAYLQMNSLRAEDTAVYYCAREPYNYNWYGMDYWGQGTLVTVSS

SEQ ID NO: 254 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSLWWPITFGQGTKVEIKRTV

SEQ ID NO: 255 - protein, 121 aa, synthetic construct
EVQLVESGGGLVQPGGSLRLSCAASGFTVSSSSIHWVRQAPGKGLEWVASIYSYSGYTSYADSVKGRFTISADTSKNTAYLQMNSLRAEDTAVYYCARHGSYGSWWALDYWGQGTLVTVSS

SEQ ID NO: 256 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYFYFPITFGQGTKVEIKRTV

SEQ ID NO: 257 - protein, 128 aa, synthetic construct
EVQLVESGGGLVQPGGSLRLSCAASGFTFYSSSIHWVRQAPGKGLEWVASISSYYGSTSYADSVKGRFTISADTSKNTAYLQMNSLRAEDTAVYYCARASYYSGYGSSYPYYMGLDYWGQGTLVTVSS

SEQ ID NO: 258 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGSYRNPLLTFGQGTKVEIKRTV

SEQ ID NO: 259 - protein, 119 aa, synthetic construct
EVQLVESGGGLVQPGGSLRLSCAASGFTFSSYSIHWVRQAPGKGLEWVASISSSSGYTSYADSVKGRFTISADTSKNTAYLQMNSLRAEDTAVYYCARMNWSHYAMDYWGQGTLVTVSS

SEQ ID NO: 260 - protein, 123 aa, synthetic construct
EVQLVESGGGLVQPGGSLRLSCAASGFTISSSSIHWVRQAPGKGLEWVAYISSYSGYTYYADSVKGRFTISADTSKNTAYLQMNSLRAEDTAVYYCARYWYGHYHSYFGLDYWGQGTLVTVSS

SEQ ID NO: 261 - protein, 122 aa, synthetic construct
EVQLVESGGGLVQPGGSLRLSCAASGFTISSSSIHWVRQAPGKGLEWVASISSYSGSTYYADSVKGRFTISADTSKNTAYLQMNSLRAEDTAVYYCARYPYGSHVYTGLDYWGQGTLVTVSS

SEQ ID NO: 262 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWNWADYLVTFGQGTKVEIKRTV

SEQ ID NO: 263 - protein, 124 aa, synthetic construct
EVQLVESGGGLVQPGGSLRLSCAASGFTISSSSIHWVRQAPGKGLEWVASIYSSSGSTSYADSVKGRFTISADTSKNTAYLQMNSLRAEDTAVYYCARVYSSRYWGWGVAFDYWGQGTLVTVSS

SEQ ID NO: 264 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYWYSLITFGQGTKVEIKRTV

SEQ ID NO: 265 - protein, 128 aa, synthetic construct
EVQLVESGGGLVQPGGSLRLSCAASGFTVYSSSIHWVRQAPGKGLEWVAYIYSSSGYTSYADSVKGRFTISADTSKNTAYLQMNSLRAEDTAVYYCARRSFPQWYNGSYTPWPAMDYWGQGTLVTVSS

SEQ ID NO: 266 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYMWWPVTFGQGTKVEIKRTV

SEQ ID NO: 267 - protein, 121 aa, synthetic construct
EVQLVESGGGLVQPGGSLRLSCAASGFTVSSSSIHWVRQAPGKGLEWVASIYSYSSYTSYADSVKGRFTISADTSKNTAYLQMNSLRAEDTAVYYCARPFYWGERYALDYWGQGTLVTVSS

SEQ ID NO: 268 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYSTLVTFGQGTKVEIKRTV

SEQ ID NO: 269 - protein, 123 aa, synthetic construct
EVQLVESGGGLVQPGGSLRLSCAASGFTFYSSSIHWVRQAPGKGLEWVASIYSSYGSTSYADSVKGRFTISADTSKNTAYLQMNSLRAEDTAVYYCARIYGWSYQGWAGMDYWGQGTLVTVSS

SEQ ID NO: 270 - protein, 124 aa, synthetic construct
EVQLVESGGGLVQPGGSLRLSCAASGFTISYSSIHWVRQAPGKGLEWVASIYPYYGSTSYADSVKGRFTISADTSKNTAYLQMNSLRAEDTAVYYCARGGDYYWGWYWVAMDYWGQGTLVTVSS

SEQ ID NO: 271 - protein, 110 aa, synthetic construct
  VARIANT 76: note = Any amino acid
DIQMTQSPSSLSASVGDRVTITCRASQSVSSAVAWYQQKPGKAPKLLIYSASSLYSGVPSRFSGSRSGTDFTLTIXSLQPEDFATYYCQKSSSSLITFGQGTKVEIKRTV

SEQ ID NO: 272 - protein, 120 aa, synthetic construct
EVQLVESGGGLVQPGGSLRLSCAASGFTFSSSYIHWVRQAPGKGLEWVASISSSYGSTSYADSVKGRFTISADTSKNTAYLQMNSLRAEDTAVYYCARMYYYTYPGMDYWGQGTLVTVSS

SEQ ID NO: 273 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQKGSSYLLTFGQGTKVEIKRTV

SEQ ID NO: 274 - protein, 124 aa, synthetic construct
EVQLVESGGGLVQPGGSLRLSCAASGFTIYSYSIHWVRQAPGKGLEWVASISPSSGYTSYADSVKGRFTISADTSKNTAYLQMNSLRAEDTAVYYCARYHYGGWSHYMSGMDYWGQGTLVTVSS

SEQ ID NO: 275 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQNYYYHKLITFGQGTKVEIKRTV

SEQ ID NO: 276 - protein, 117 aa, synthetic construct
EVQLVESGGGLVQPGGSLRLSCAASGFTFSYSSIHWVRQAPGKGLEWVASISSSYGYTSYADSVKGRFTISADTSKNTAYLQMNSLRAEDTAVYYCARGRYGGMDYWGQGTLVTVSS

SEQ ID NO: 277 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQLSYVYKLITFGQGTKVEIKRTV

SEQ ID NO: 278 - protein, 117 aa, synthetic construct
EVQLVESGGGLVQPGGSLRLSCAASGFTFYSSSIHWVRQAPGKGLEWVASISSSYGYTSYADSVKGRFTISADTSKNTAYLQMNSLRAEDTAVYYCARGWYKAMDYWGQGTLVTVSS

SEQ ID NO: 279 - protein, 120 aa, synthetic construct
EVQLVESGGGLVQPGGSLRLSCAASGFTFSYSSIHWVRQAPGKGLEWVASISSSYGSTYYADSVKGRFTISADTSKNTAYLQMNSLRAEDTAVYYCARMYYYYYPGIDYWGQGTLVTVSS

SEQ ID NO: 280 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDLATYYCQQYYYFPITFGQGTKVEIKRTV

SEQ ID NO: 281 - protein, 125 aa, synthetic construct
EVQLVESGGGLVQPGGSLRLSCAASGFTFSSSSIHWVRQAPGKGLEWVASISPYYGYTSYADSVKGRFTISADTSKNTAYLQMNSLRAEDTAVYYCARSPYYWYQYFYGWGLDYWGQGTLVTVSS

SEQ ID NO: 282 - protein, 122 aa, synthetic construct
EVQLVESGGGLVQPGGSLRLSCAASGFTFSYSSIHWVRQAPGKGLEWVASISSSSGSTSYADSVKGRFTISADTSKNTAYLQMNSLRAEDTAVYYCARSPYWWNYMSAMDYWGQGTLVTVSS

SEQ ID NO: 283 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGWWWPFTFGQGTKVEIKRTV

SEQ ID NO: 284 - protein, 125 aa, synthetic construct
EVQLVESGGGLVQPGGSLRLSCAASGFTVSSYSIHWVRQAPGKGLEWVASISPYYGYTSYADSVKGRFTISADTSKNTAYLQMNSLRAEDTAVYYCARWSWQYYSGHSSWGLDYWGQGTLVTVSS

SEQ ID NO: 285 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSWYFPLTFGQGTKVEIKRTV

SEQ ID NO: 286 - protein, 125 aa, synthetic construct
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWYNEYYHDYYWDAMDYWGQGTLVTVSS

SEQ ID NO: 287 - protein, 120 aa, synthetic construct
EVQLVESGGGLVQPGGSLRLSCAASGFTLYYSSIHWVRQAPGKGLEWVASISSSSGSTSYADSVKGRFTISADTSKNTAYLQMNSLRAEDTAVYYCARWMYWWSFALDYWGQGTLVTVSS

SEQ ID NO: 288 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYLWPITFGQGTKVEIKRTV

SEQ ID NO: 289 - protein, 122 aa, synthetic construct
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWQYHYNYWYGMDYWGQGTLVTVSS

SEQ ID NO: 290 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYPMSLITFGQGTKVEIKRTV

SEQ ID NO: 291 - protein, 119 aa, synthetic construct
EVQLVESGGGLVQPGGSLRLSCAASGFTVSYSSIHWVRQAPGKGLEWVASISPYSGYTSYADSVKGRFTISADTSKNTAYLQMNSLRAEDTAVYYCARGYDYYAGLDYWGQGTLVTVSS

SEQ ID NO: 292 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYYFPITFGQGTKVEIKRTV

SEQ ID NO: 293 - protein, 127 aa, synthetic construct
EVQLVESGGGLVQPGGSLRLSCAASGFTFSYYSIHWVRQAPGKGLEWVASISPYYGYTSYADSVKGRFTISADTSKNTAYLQMNSLRAEDTAVYYCARWESEYSGTYEDYWAGMDYWGQGTLVTVSS

SEQ ID NO: 294 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYMWWPITFGQGTKVEIKRTV

SEQ ID NO: 295 - protein, 121 aa, synthetic construct
EVQLVESGGGLVQPGGSLRLSCAASGFTISYSSIHWVRQAPGKGLEWVASISSSYSYTSYADSVKGRFTISADTSKNTAYLQMNSLRAEDTAVYYCARTGYWQGYLALDYWGQGTLVTVSS

SEQ ID NO: 296 - protein, 123 aa, synthetic construct
EVQLVESGGGLVQPGGSLRLSCAASGFTISYSSIHWVRQAPGKGLEWVASISSSSGSTSYADSVKGRFTISADTSKNTAYLQMNSLRAEDTAVYYCARTYYYYWNSTPAMDYWGQGTLVTVSS

SEQ ID NO: 297 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYGYPVTFGQGTKVEIKRTV

SEQ ID NO: 298 - protein, 129 aa, synthetic construct
EVQLVESGGGLVQPGGSLRLSCAASGFTFSSSSIHWVRQAPGKGLEWVASISSSYGYTSYADSVKGRFTISADTSKNTAYLQMNSLRAEDTAVYYCARWYNSSWYYSNWWYKGFGMDYWGQGTLVTVSS

SEQ ID NO: 299 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYSSLFTFGQGTKVEIKRTV

SEQ ID NO: 300 - protein, 124 aa, synthetic construct
EVQLVESGGGLVQPGGSLRLSCAASGFTFYSSSIHWVRQAPGKGLEWVASISSSYGYTSYADSVKGRFTISADTSKNTAYLQMNSLRAEDTAVYYCARTSYTYPVYTYYGFDYWGQGTLVTVSS

SEQ ID NO: 301 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSWYYPLTFGQGTKVEIKRTV

SEQ ID NO: 302 - protein, 122 aa, synthetic construct
EVQLVESGGGLVQPGGSLRLSCAASGFTLYSSSIHWVRQAPGKGLEWVASISSSYGYTSYADSVKGRFTISADTSKNTAYLQMNSLRAEDTAVYYCARYRYSSWNRGAIDYWGQGTLVTVSS

SEQ ID NO: 303 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWWPLTFGQGTKVEIKRTV

SEQ ID NO: 304 - protein, 122 aa, synthetic construct
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWSKSPWYYQGIDYWGQGTLVTVSS

SEQ ID NO: 305 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYHYWASLITFGQGTKVEIKRTV

SEQ ID NO: 306 - protein, 123 aa, synthetic construct
EVQLVESGGGLVQPGGSLRLSCAASGFTVSSSSIHWVRQAPGKGLEWVASIYSYSGSTYYADSVKGRFTISADTSKNTAYLQMNSLRAEDTAVYYCARQYSSSYYVWPGMDYWGQGTLVTVSS

SEQ ID NO: 307 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWWKSLVTFGQGTKVEIKRTV

SEQ ID NO: 308 - protein, 126 aa, synthetic construct
EVQLVESGGGLVQPGGSLRLSCAASGFTLSSSSIHWVRQAPGKGLEWVASISSYYGSTSYADSVKGRFTISADTSKNTAYLQMNSLRAEDTAVYYCARMHYSWQEYYSYDWGMDYWGQGTLVTVSS

SEQ ID NO: 309 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQPYYPLITFGQGTKVEIKRTV

SEQ ID NO: 310 - protein, 120 aa, synthetic construct
EVQLVESGGGLVQPGGSLRLSCAASGFTISYSSIHWVRQAPGKGLEWVASIYPSYGSTSYADSVKGRFTISADTSKNTAYLQMNSLRAEDTAVYYCARWQGYYQPALDYWGQGTLVTVSS

SEQ ID NO: 311 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSKYYYPITFGQGTKVEIKRTV

SEQ ID NO: 312 - protein, 121 aa, synthetic construct
EVQLVESGGGLVQPGGSLRLSCAASGFTISSSSIHWVRQAPGKGLEWVASISPYYGYTSYADSVKGRFTISADTSKNTAYLQMNSLRAEDTAVYYCARWGYGWYWYGLDYWGQGTLVTVSS

SEQ ID NO: 313 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGHDMNPVTFGQGTKVEIKRTV

SEQ ID NO: 314 - protein, 120 aa, synthetic construct
EVQLVESGGGLVQPGGSLRLSCAASGFTLSSSSIHWVRQAPGKGLEWVASIYSSYGYTYYADSVKGRFTISADTSKNTAYLQMNSLRAEDTAVYYCARYYYYWYGGMDYWGQGTLVTVSS

SEQ ID NO: 315 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSWMSDSLITFGQGTKVEIKRTV

SEQ ID NO: 316 - protein, 123 aa, synthetic construct
EVQLVESGGGLVQPGGSLRLSCAASGFTFSYSSIHWVRQAPGKGLEWVASIYPSSGYTSYADSVKGRFTISADTSKNTAYLQMNSLRAEDTAVYYCARGWWYWMAWDYAMDYWGQGTLVTVSS

SEQ ID NO: 317 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQMQYSGWLITFGQGTKVEIKRTV

SEQ ID NO: 318 - protein, 130 aa, synthetic construct
EVQLVESGGGLVQPGGSLRLSCAASGFTISSSSIHWVRQAPGKGLEWVASISSYYGYTSYADSVKGRFTISADTSKNTAYLQMNSLRAEDTAVYYCARYYSYSSGYGYYDYFDWGAMDYWGQGTLVTVSS

SEQ ID NO: 319 - protein, 129 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYYGYVWGGYWGWWYSKALDYWGQGTLVTVSS

SEQ ID NO: 320 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYDWNYYLVTFGQGTKVEIKRTV

SEQ ID NO: 321 - protein, 126 aa, synthetic construct
EVQLVESGGGLVQPGGSLRLSCAASGFTIYSSSIHWVRQAPGKGLEWVASISSYYGYTSYADSVKGRFTISADTSKNTAYLQMNSLRAEDTAVYYCARYQYYGSLYYSQQWAMDYWGQGTLVTVSS

SEQ ID NO: 322 - protein, 129 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSPSSPYFMSWGWYWQYGIDYWGQGTLVTVSS

SEQ ID NO: 323 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSWGGLVTFGQGTKVEIKRTV

SEQ ID NO: 324 - protein, 121 aa, synthetic construct
EVQLVESGGGLVQPGGSLRLSCAASGFTFSSSYIHWVRQAPGKGLEWVASISPSYGYTSYADSVKGRFTISADTSKNTAYLQMNSLRAEDTAVYYCARDMYEWWHWAIDYWGQGTLVTVSS

SEQ ID NO: 325 - protein, 129 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYGHYLYYWGWGWYWSAALDYWGQGTLVTVSS

SEQ ID NO: 326 - protein, 514 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVYSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSYSIHWVRQAPGKGLEWVASIYSSYGYTSYADSVKGRFTISADTSKNTAYLQMNSLRAEDTAVYYCARGGWYPAMDYWGQGTLVTVSSLEGGGGLNDIFEAQKIEWHESRHHHHHH

SEQ ID NO: 327 - protein, 517 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSSIHWVRQAPGKGLEWVAYISSYSGYTYYADSVKGRFTISADTSKNTAYLQMNSLRAEDTAVYYCARYWYGHYHSYFGLDYWGQGTLVTVSSLEGGGGLNDIFEAQKIEWHESRHHHHHH

SEQ ID NO: 328 - protein, 11 aa, synthetic construct
QQISYVHRLVT

SEQ ID NO: 329 - protein, 516 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSWGGLVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSSYIHWVRQAPGKGLEWVASISPSYGYTSYADSVKGRFTISADTSKNTAYLQMNSLRAEDTAVYYCARDMYEWWHWAIDYWGQGTLVTVSSLEGGGGLNDIFEAQKIEWHESRHHHHHH

SEQ ID NO: 330 - protein, 514 aa, synthetic construct
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSLEGGGGLNDIFEAQKIEWHESRHHHHHH

SEQ ID NO: 331 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 671 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 333 - protein, 446 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 334 - protein, 212 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 335 - protein, 9 aa, synthetic construct
GPWRPAMDY

SEQ ID NO: 336 - protein, 6 aa, synthetic construct
SASSLY

SEQ ID NO: 337 - protein, 11 aa, synthetic construct
QQTSYVRRKLT

SEQ ID NO: 338 - protein, 9 aa, synthetic construct
GPWYPAMDY

SEQ ID NO: 339 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVAKTITFGQGTKVEIKRTV

SEQ ID NO: 340 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQVSYVAKAVTFGQGTKVEIKRTV

SEQ ID NO: 341 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGEWYAAMDYWGQGTLVTVSS

SEQ ID NO: 342 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVIRALTFGQGTKVEIKRTV

SEQ ID NO: 343 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGLWSAAMDYWGQGTLVTVSS

SEQ ID NO: 344 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQTSYVARSITFGQGTKVEIKRTV

SEQ ID NO: 345 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGPWYAAMDYWGQGTLVTVSS

SEQ ID NO: 346 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQVSYVKREITFGQGTKVEIKRTV

SEQ ID NO: 347 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGSWAAAMDYWGQGTLVTVSS

SEQ ID NO: 348 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQVSYVIRELTFGQGTKVEIKRTV

SEQ ID NO: 349 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGLWTAAMDYWGQGTLVTVSS

SEQ ID NO: 350 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVIRTVTFGQGTKVEIKRTV

SEQ ID NO: 351 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGHWTPAMDYWGQGTLVTVSS

SEQ ID NO: 352 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVHRLVTFGQGTKVEIKRTV

SEQ ID NO: 353 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGPWRPAMDYWGQGTLVTVSS

SEQ ID NO: 354 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVRREITFGQGTKVEIKRTV

SEQ ID NO: 355 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGHWHAAMDYWGQGTLVTVSS

SEQ ID NO: 356 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQTSYVRRKLTFGQGTKVEIKRTV

SEQ ID NO: 357 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGPWYPAMDYWGQGTLVTVSS

SEQ ID NO: 358 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQPSYVRRSITFGQGTKVEIKRTV

SEQ ID NO: 359 - protein, 118 aa, synthetic construct
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWKPAMDYWGQGTLVTVSS

SEQ ID NO: 360 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASYVRRVVTFGQGTKVEIKRTV

SEQ ID NO: 361 - protein, 101 aa, synthetic construct
GSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 362 - protein, 13 aa, synthetic construct
SASSLYSSASSLY

SEQ ID NO: 363 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTVEIKRTV

SEQ ID NO: 364 - protein, 11 aa, synthetic construct
QQPSYVRRSIT

SEQ ID NO: 365 - protein, 10 aa, synthetic construct
QQSGWSYPIT

SEQ ID NO: 366 - protein, 17 aa, synthetic construct
SISPYYGSTSYADSVKG

SEQ ID NO: 367 - protein, 6 aa, synthetic construct
YYALDY

SEQ ID NO: 368 - protein, 11 aa, synthetic construct
QQISYVYSLIT

SEQ ID NO: 369 - protein, 5 aa, synthetic construct
SYSIH

SEQ ID NO: 370 - protein, 17 aa, synthetic construct
SIYSSYGYTSYADSVKG

SEQ ID NO: 371 - protein, 11 aa, synthetic construct
QQYQYGYNLIT

SEQ ID NO: 372 - protein, 5 aa, synthetic construct
YSSIH

SEQ ID NO: 373 - protein, 17 aa, synthetic construct
SIYSYSGSTSYADSVKG

SEQ ID NO: 374 - protein, 15 aa, synthetic construct
YSYGWVGPGWRAIDY

SEQ ID NO: 375 - protein, 11 aa, synthetic construct
QQSSSVYKLLT

SEQ ID NO: 376 - protein, 17 aa, synthetic construct
SISSSYSYTSYADSVKG

SEQ ID NO: 377 - protein, 11 aa, synthetic construct
GGPGWYRAMDY

SEQ ID NO: 378 - protein, 9 aa, synthetic construct
QQSSSSLIT

SEQ ID NO: 379 - protein, 5 aa, synthetic construct
SSSIH

SEQ ID NO: 380 - protein, 14 aa, synthetic construct
GYFYYGWWAMAFDY

SEQ ID NO: 381 - protein, 11 aa, synthetic construct
QQSQWYEPLIT

SEQ ID NO: 382 - protein, 18 aa, synthetic construct
SYSYMSQWGWYQYSGMDY

SEQ ID NO: 383 - protein, 11 aa, synthetic construct
QQGSYTYRLIT

SEQ ID NO: 384 - protein, 17 aa, synthetic construct
SISSSSGYTSYADSVKG

SEQ ID NO: 385 - protein, 10 aa, synthetic construct
YAWWAHGLDY

SEQ ID NO: 386 - protein, 11 aa, synthetic construct
QQASYWYNLFT

SEQ ID NO: 387 - protein, 14 aa, synthetic construct
QYSMHFPWGYGMDY

SEQ ID NO: 388 - protein, 9 aa, synthetic construct
QQSDMPPIT

SEQ ID NO: 389 - protein, 17 aa, synthetic construct
YIYSSSGYTSYADSVKG

SEQ ID NO: 390 - protein, 12 aa, synthetic construct
PVNYYYQGALDY

SEQ ID NO: 391 - protein, 9 aa, synthetic construct
QQYYVFPIT

SEQ ID NO: 392 - protein, 17 aa, synthetic construct
SISPSSGYTYYADSVKG

SEQ ID NO: 393 - protein, 21 aa, synthetic construct
YHYMFEYDKGESKWGYYGFDY

SEQ ID NO: 394 - protein, 9 aa, synthetic construct
QQSQYFPIT

SEQ ID NO: 395 - protein, 17 aa, synthetic construct
SIYSYYGYTSYADSVKG

SEQ ID NO: 396 - protein, 13 aa, synthetic construct
NSWSWYSGVGMDY

SEQ ID NO: 397 - protein, 17 aa, synthetic construct
SISSYSSSTYYADSVKG

SEQ ID NO: 398 - protein, 14 aa, synthetic construct
YPYGWGWGGSGLDY

SEQ ID NO: 399 - protein, 10 aa, synthetic construct
QQFDFQYLIT

SEQ ID NO: 400 - protein, 17 aa, synthetic construct
SIYSYYGSTYYADSVKG

SEQ ID NO: 401 - protein, 8 aa, synthetic construct
GEKWALDY

SEQ ID NO: 402 - protein, 11 aa, synthetic construct
QQYMYYQPLIT

SEQ ID NO: 403 - protein, 12 aa, synthetic construct
EPYNYNWYGMDY

SEQ ID NO: 404 - protein, 9 aa, synthetic construct
QQSLWWPIT

SEQ ID NO: 405 - protein, 17 aa, synthetic construct
SIYSYSGYTSYADSVKG

SEQ ID NO: 406 - protein, 12 aa, synthetic construct
HGSYGSWWALDY

SEQ ID NO: 407 - protein, 9 aa, synthetic construct
QQYFYFPIT

SEQ ID NO: 408 - protein, 17 aa, synthetic construct
SISSYYGSTSYADSVKG

SEQ ID NO: 409 - protein, 19 aa, synthetic construct
ASYYSGYGSSYPYYMGLDY

SEQ ID NO: 410 - protein, 11 aa, synthetic construct
QQGSYRNPLLT

SEQ ID NO: 411 - protein, 10 aa, synthetic construct
MNWSHYAMDY

SEQ ID NO: 412 - protein, 17 aa, synthetic construct
YISSYSGYTYYADSVKG

SEQ ID NO: 413 - protein, 14 aa, synthetic construct
YWYGHYHSYFGLDY

SEQ ID NO: 414 - protein, 17 aa, synthetic construct
SISSYSGSTYYADSVKG

SEQ ID NO: 415 - protein, 13 aa, synthetic construct
YPYGSHVYTGLDY

SEQ ID NO: 416 - protein, 11 aa, synthetic construct
QQWNWADYLVT

SEQ ID NO: 417 - protein, 17 aa, synthetic construct
SIYSSSGSTSYADSVKG

SEQ ID NO: 418 - protein, 15 aa, synthetic construct
VYSSRYWGWGVAFDY

SEQ ID NO: 419 - protein, 10 aa, synthetic construct
QQYYWYSLIT

SEQ ID NO: 420 - protein, 19 aa, synthetic construct
RSFPQWYNGSYTPWPAMDY

SEQ ID NO: 421 - protein, 9 aa, synthetic construct
QQYMWWPVT

SEQ ID NO: 422 - protein, 17 aa, synthetic construct
SIYSYSSYTSYADSVKG

SEQ ID NO: 423 - protein, 12 aa, synthetic construct
PFYWGERYALDY

SEQ ID NO: 424 - protein, 9 aa, synthetic construct
QQSYSTLVT

SEQ ID NO: 425 - protein, 17 aa, synthetic construct
SIYSSYGSTSYADSVKG

SEQ ID NO: 426 - protein, 14 aa, synthetic construct
IYGWSYQGWAGMDY

SEQ ID NO: 427 - protein, 17 aa, synthetic construct
SIYPYYGSTSYADSVKG

SEQ ID NO: 428 - protein, 15 aa, synthetic construct
GGDYYWGWYWVAMDY

SEQ ID NO: 429 - protein, 9 aa, synthetic construct
QKSSSSLIT

SEQ ID NO: 430 - protein, 17 aa, synthetic construct
SISSSYGSTSYADSVKG

SEQ ID NO: 431 - protein, 11 aa, synthetic construct
MYYYTYPGMDY

SEQ ID NO: 432 - protein, 9 aa, synthetic construct
QKGSSYLLT

SEQ ID NO: 433 - protein, 17 aa, synthetic construct
SISPSSGYTSYADSVKG

SEQ ID NO: 434 - protein, 15 aa, synthetic construct
YHYGGWSHYMSGMDY

SEQ ID NO: 435 - protein, 11 aa, synthetic construct
QQNYYYHKLIT

SEQ ID NO: 436 - protein, 17 aa, synthetic construct
SISSSYGYTSYADSVKG

SEQ ID NO: 437 - protein, 8 aa, synthetic construct
GRYGGMDY

SEQ ID NO: 438 - protein, 11 aa, synthetic construct
QQLSYVYKLIT

SEQ ID NO: 439 - protein, 8 aa, synthetic construct
GWYKAMDY

SEQ ID NO: 440 - protein, 17 aa, synthetic construct
SISSSYGSTYYADSVKG

SEQ ID NO: 441 - protein, 11 aa, synthetic construct
MYYYYYPGIDY

SEQ ID NO: 442 - protein, 9 aa, synthetic construct
QQYYYFPIT

SEQ ID NO: 443 - protein, 17 aa, synthetic construct
SISPYYGYTSYADSVKG

SEQ ID NO: 444 - protein, 16 aa, synthetic construct
SPYYWYQYFYGWGLDY

SEQ ID NO: 445 - protein, 13 aa, synthetic construct
SPYWWNYMSAMDY

SEQ ID NO: 446 - protein, 9 aa, synthetic construct
QQGWWWPFT

SEQ ID NO: 447 - protein, 16 aa, synthetic construct
WSWQYYSGHSSWGLDY

SEQ ID NO: 448 - protein, 9 aa, synthetic construct
QQSWYFPLT

SEQ ID NO: 449 - protein, 16 aa, synthetic construct
WYNEYYHDYYWDAMDY

SEQ ID NO: 450 - protein, 11 aa, synthetic construct
WMYWWSFALDY

SEQ ID NO: 451 - protein, 9 aa, synthetic construct
QQSYLWPIT

SEQ ID NO: 452 - protein, 13 aa, synthetic construct
WQYHYNYWYGMDY

SEQ ID NO: 453 - protein, 9 aa, synthetic construct
QQYPMSLIT

SEQ ID NO: 454 - protein, 17 aa, synthetic construct
SISPYSGYTSYADSVKG

SEQ ID NO: 455 - protein, 10 aa, synthetic construct
GYDYYAGLDY

SEQ ID NO: 456 - protein, 5 aa, synthetic construct
YYSIH

SEQ ID NO: 457 - protein, 12 aa, synthetic construct
WESEYSGTYEDY

SEQ ID NO: 458 - protein, 9 aa, synthetic construct
QQYMWWPIT

SEQ ID NO: 459 - protein, 12 aa, synthetic construct
TGYWQGYLALDY

SEQ ID NO: 460 - protein, 14 aa, synthetic construct
TYYYYWNSTPAMDY

SEQ ID NO: 461 - protein, 9 aa, synthetic construct
QQSYGYPVT

SEQ ID NO: 462 - protein, 20 aa, synthetic construct
WYNSSWYYSNWWYKGFGMDY

SEQ ID NO: 463 - protein, 9 aa, synthetic construct
QQYYSSLFT

SEQ ID NO: 464 - protein, 15 aa, synthetic construct
TSYTYPVYTYYGFDY

SEQ ID NO: 465 - protein, 9 aa, synthetic construct
QQSWYYPLT

SEQ ID NO: 466 - protein, 13 aa, synthetic construct
YRYSSWNRGAIDY

SEQ ID NO: 467 - protein, 9 aa, synthetic construct
QQSYWWPLT

SEQ ID NO: 468 - protein, 13 aa, synthetic construct
WSKSPWYYQGIDY

SEQ ID NO: 469 - protein, 11 aa, synthetic construct
QQYHYWASLIT

SEQ ID NO: 470 - protein, 17 aa, synthetic construct
SIYSYSGSTYYADSVKG

SEQ ID NO: 471 - protein, 14 aa, synthetic construct
QYSSSYYVWPGMDY

SEQ ID NO: 472 - protein, 11 aa, synthetic construct
QQSYWWKSLVT

SEQ ID NO: 473 - protein, 17 aa, synthetic construct
MHYSWQEYYSYDWGMDY

SEQ ID NO: 474 - protein, 9 aa, synthetic construct
QQPYYPLIT

SEQ ID NO: 475 - protein, 17 aa, synthetic construct
SIYPSYGSTSYADSVKG

SEQ ID NO: 476 - protein, 11 aa, synthetic construct
WQGYYQPALDY

SEQ ID NO: 477 - protein, 11 aa, synthetic construct
QQSSKYYYPIT

SEQ ID NO: 478 - protein, 12 aa, synthetic construct
WGYGWYWYGLDY

SEQ ID NO: 479 - protein, 10 aa, synthetic construct
QQGHDMNPVT

SEQ ID NO: 480 - protein, 17 aa, synthetic construct
SIYSSYGYTYYADSVKG

SEQ ID NO: 481 - protein, 11 aa, synthetic construct
YYYYWYGGMDY

SEQ ID NO: 482 - protein, 11 aa, synthetic construct
QQSWMSDSLIT

SEQ ID NO: 483 - protein, 17 aa, synthetic construct
SIYPSSGYTSYADSVKG

SEQ ID NO: 484 - protein, 14 aa, synthetic construct
GWWYWMAWDYAMDY

SEQ ID NO: 485 - protein, 11 aa, synthetic construct
QQMQYSGWLIT

SEQ ID NO: 486 - protein, 17 aa, synthetic construct
SISSYYGYTSYADSVKG

SEQ ID NO: 487 - protein, 21 aa, synthetic construct
YYSYSSGYGYYDYFDWGAMDY

SEQ ID NO: 488 - protein, 11 aa, synthetic construct
RASQDIRNYLN

SEQ ID NO: 489 - protein, 7 aa, synthetic construct
YTSRLHS

SEQ ID NO: 490 - protein, 9 aa, synthetic construct
QQGNTLPWT

SEQ ID NO: 491 - protein, 5 aa, synthetic construct
GYTMN

SEQ ID NO: 492 - protein, 17 aa, synthetic construct
LINPYKGVSTYNQKFKD

SEQ ID NO: 493 - protein, 13 aa, synthetic construct
SGYYGDSDWYFDV

SEQ ID NO: 494 - protein, 9 aa, synthetic construct
QQDWYFPIT

SEQ ID NO: 495 - protein, 9 aa, synthetic construct
FTFSSYYIH

SEQ ID NO: 496 - protein, 17 aa, synthetic construct
SISPSSGSTYYADSVKG

SEQ ID NO: 497 - protein, 21 aa, synthetic construct
YGGRSYWQKQDSYFYQHGLDY

SEQ ID NO: 498 - protein, 9 aa, synthetic construct
FTFSSSSIH

SEQ ID NO: 499 - protein, 17 aa, synthetic construct
SISSYSGYTSYADSVKG

SEQ ID NO: 500 - protein, 18 aa, synthetic construct
SYSYSEFRYYYSGQGMDY

SEQ ID NO: 501 - protein, 14 aa, synthetic construct
SNYGWRWHLVGMDY

SEQ ID NO: 502 - protein, 8 aa, synthetic construct
TFSSSSIH

SEQ ID NO: 503 - protein, 14 aa, synthetic construct
SPYVYYWYMVGFDY

SEQ ID NO: 504 - protein, 14 aa, synthetic construct
TGTSSDVGGYNYVS

SEQ ID NO: 505 - protein, 7 aa, synthetic construct
DVSKRPS

SEQ ID NO: 506 - protein, 10 aa, synthetic construct
GSYADTDTIV

SEQ ID NO: 507 - protein, 5 aa, synthetic construct
TDAIT

SEQ ID NO: 508 - protein, 17 aa, synthetic construct
GIIPLLDSVDYAQRFQG

SEQ ID NO: 509 - protein, 10 aa, synthetic construct
WSSVDTGLDY

SEQ ID NO: 510 - protein, 7 aa, synthetic construct
NDPHYWG

SEQ ID NO: 511 - protein, 16 aa, synthetic construct
STSHSGHTYFNPSLKS

SEQ ID NO: 512 - protein, 16 aa, synthetic construct
MRYYYSGTYPVYYFDY

SEQ ID NO: 513 - protein, 13 aa, synthetic construct
SGSSSNIGSNFVS

SEQ ID NO: 514 - protein, 7 aa, synthetic construct
SNNQRPS

SEQ ID NO: 515 - protein, 11 aa, synthetic construct
AAWDDSLNGPV

SEQ ID NO: 516 - protein, 5 aa, synthetic construct
RYGVS

SEQ ID NO: 517 - protein, 17 aa, synthetic construct
GIIPMFGTANYAQKFQG

SEQ ID NO: 518 - protein, 12 aa, synthetic construct
GDNSAYSDAFNI

SEQ ID NO: 519 - protein, 20 aa, synthetic construct
YYGYVWGGYWGWWYSKALDY

SEQ ID NO: 520 - protein, 11 aa, synthetic construct
QQYDWNYYLVT

SEQ ID NO: 521 - protein, 17 aa, synthetic construct
YQYYGSLYYSQQWAMDY

SEQ ID NO: 522 - protein, 20 aa, synthetic construct
SPSSPYFMSWGWYWQYGIDY

SEQ ID NO: 523 - protein, 10 aa, synthetic construct
QQSSWGGLVT

SEQ ID NO: 524 - protein, 17 aa, synthetic construct
SISPSYGYTSYADSVKG

SEQ ID NO: 525 - protein, 12 aa, synthetic construct
DMYEWWHWAIDY

SEQ ID NO: 526 - protein, 20 aa, synthetic construct
YGHYLYYWGWGWYWSAALDY

SEQ ID NO: 527 - protein, 11 aa, synthetic construct
QQVSYVAKAVT

SEQ ID NO: 528 - protein, 9 aa, synthetic construct
GEWYAAMDY

SEQ ID NO: 529 - protein, 11 aa, synthetic construct
QQISYVIRALT

SEQ ID NO: 530 - protein, 9 aa, synthetic construct
GLWSAAMDY

SEQ ID NO: 531 - protein, 11 aa, synthetic construct
QQTSYVARSIT

SEQ ID NO: 532 - protein, 9 aa, synthetic construct
GPWYAAMDY

SEQ ID NO: 533 - protein, 11 aa, synthetic construct
QQVSYVKREIT

SEQ ID NO: 534 - protein, 9 aa, synthetic construct
GSWAAAMDY

SEQ ID NO: 535 - protein, 11 aa, synthetic construct
QQVSYVIRELT

SEQ ID NO: 536 - protein, 9 aa, synthetic construct
GLWTAAMDY

SEQ ID NO: 537 - protein, 11 aa, synthetic construct
QQISYVIRTVT

(19) United States
(12) Patent Application Publication
(10) Pub. No.: US 2025/0353912 A1
(43) Pub. Date: Nov. 20, 2025

(54) COMPOSITIONS AND METHODS COMPRISING ANTIBODIES THAT BIND TO COVALENT PEPTIDE CONJUGATES

(71) Applicant: New York University, New York, NY (US)

(72) Inventors: Shohei KOIDE, New York, NY (US); Lorenzo MASO, New York, NY (US); Benjamin G. NEEL, New York, NY (US); Akiko KOIDE, New York, NY (US); Takamitsu HATTORI, West New York, NJ (US)

(21) Appl. No.: 18/876,523

(22) PCT Filed: Jun. 21, 2023
(86) PCT No.: PCT/US2023/068831

§ 371 Date: Dec. 18, 2024

Related U.S. Application Data
(60) Provisional application No. 63/366,819, filed on Jun. 22, 2022.
(60) Provisional application No. 63/369,702, filed on Jul. 28, 2022.
(60) Provisional application No. 63/402,606, filed on Aug. 31, 2022.
(60) Provisional application No. 63/377,466, filed on Sep. 28, 2022.
(60) Provisional application No. 63/485,788, filed on Feb. 17, 2023.

(51) Int. Cl. C07K 16/28; A61K 45/06; A61K 47/64; A61P 35/00; C12N 15/86; G01N 33/543; G01N 33/569; G01N 33/573; G01N 33/68

(52) U.S. Cl. CPC C07K 16/2833; A61K 45/06; A61K 47/6425; A61P 35/00; C07K 16/2809; C12N 15/86; G01N 33/543; G01N 33/56972; G01N 33/573; G01N 33/6872; C07K 2317/31; C07K 2317/32; C07K 2317/565; C07K 2317/92; G01N 2333/7051; G01N 2333/70539; G01N 2333/71; G01N 2333/912

(57) ABSTRACT

Provided are compositions and methods that include binding partners that specifically bind to a peptide conjugate/MHC complex comprising a peptide conjugate that is formed by the covalent reaction of a targeted covalent inhibitor with a peptide. The binding partners are provided as antibodies and antibody derivatives that specifically bind to the peptide conjugate/MHC complexes.

### CROSS-REFERENCE TO RELATED APPLICATIONS

This application claims the benefit of U.S. Provisional Application No. 63/366,819, filed Jun. 22, 2022; U.S. Provisional Application No. 63/369,702, filed Jul. 28, 2022; U.S. Provisional Application No. 63/402,606, filed Aug. 31, 2022; U.S. Provisional Application No. 63/377,466, filed Sep. 28, 2022, and U.S. Provisional Application No. 63/485,788, filed Feb. 17, 2023, each of which is incorporated herein by reference in its entirety.

### STATEMENT REGARDING FEDERALLY SPONSORED RESEARCH

This invention was made with government support under grant nos. CA016087 and CA267362 awarded by the National Institutes of Health. The government has certain rights in the invention.

### BACKGROUND

There is an ongoing and unmet need for agents that can bind to targets that include drugs that are covalently bound to proteins or peptides. In particular, there is a need to improve the efficacy of targeted therapy and also to increase tumor immunogenicity and the efficacy of immune therapy against cancer driven by intracellular oncogenes and loss of tumor suppressor genes. The disclosure is pertinent to these needs.

### BRIEF SUMMARY

The present disclosure provides compositions and methods that include binding partners that bind with specificity to target sites on proteins or peptides that comprise a covalently attached molecule. It is believed that this is the first disclosure of binding partners with this binding function. The disclosure illustrates this approach using binding partners in the form of numerous antibodies and antibody derivatives that specifically bind to proteins and peptides that have been covalently modified by attachment of a molecule, wherein the molecules are illustrated by a variety of drugs. Further, the disclosure demonstrates binding partners that bind with specificity to peptides that have been covalently modified by attachment of a small molecule drug are specific for the described covalently modified peptides when presented in the context of a human leukocyte antigen (HLA), wherein HLA is a representative example of a major histocompatibility complex (MHC). Thus, binding partners that are specific for peptide-drug conjugates in an HLA complex are demonstrated. The disclosure includes polynucleotides encoding the described binding partners and cells that are modified to express the binding partners. The disclosure includes diagnostic, prophylactic and therapeutic approaches using the binding partners.

In an aspect, the present disclosure provides a binding partner that specifically binds to a peptide conjugate/MHC complex, wherein the peptide conjugate/MHC complex comprises: (a) a peptide conjugate formed by the covalent reaction of a targeted covalent inhibitor or fragment thereof with a peptide; and (b) an MHC.

In some embodiments, the binding partner binds to the peptide conjugate/MHC complex with a greater affinity than to the peptide or free targeted covalent inhibitor. In some embodiments, the affinity of the binding partner for the peptide conjugate/MHC complex is 100-10,000 times greater than the affinity of the binding partner for the peptide or free targeted covalent inhibitor.

In some embodiments, the MHC is a human leukocyte antigen (HLA), optionally wherein the HLA is an HLA-A, HLA-B, or HLA-C. In some embodiments, the HLA molecule is an HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, and/or HLA-B*15:01 molecule.

In some embodiments, the peptide comprises a nucleophilic or an electrophilic residue, said residue optionally being one of cysteine, lysine, tyrosine, histidine, serine, arginine, or threonine.

In some embodiments, the peptide comprises a cysteine residue.

In some embodiments, the peptide conjugate is formed by a covalent reaction between the targeted covalent inhibitor and a cysteine residue in the peptide.

In some embodiments, the peptide is a segment of a protein that is associated with a cancer, optionally wherein the protein is encoded by a gene that is mutated in a cancer.

In some embodiments, the peptide is a segment of an enzyme, and wherein the targeted covalent inhibitor is an inhibitor of the enzyme. In some embodiments, the enzyme is a kinase or a GTPase.

In some embodiments, the peptide is or is derived from RAS (e.g., KRAS, HRAS, or NRAS), Bruton's tyrosine kinase (BTK), any epidermal growth factor receptor (EGFR) family member that is selected from EGFR (ERBB1), HER2/NEU (ERBB2), HER3 (ERBB3), and HER4 (ERBB4); MET (HGFR); any fibroblast growth factor receptor (FGFR); any cyclin-dependent kinase (CDK); Acetylcholine Esterase (ACHE); p90 ribosomal S6 kinase (RSK); TP53, IDH1, GNAS, FBXW7, CTNNB1, DNMT3A, a cathepsin that is selected from cathepsin B, C, F, H, K, L, O, S, V, W and X; any caspase; a protein involved in obesity that is optionally Pancreatic lipase or METAP2; any cancer testis antigen (CTA); a long interspersed element-1 (LINE-1); a short interspersed element that is optionally Alu; and any endogenous retroviral protein, and optionally wherein the RAS is KRAS, HRAS, or NRAS.

In some embodiments, the peptide comprises a segment of KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}.

In some embodiments, the peptide comprises the amino acid sequence of

VVVGACGVGK, VVGACGVGK, or KLVVVGACGV.

In some embodiments, the targeted covalent inhibitor is (i) a tri-complex KRAS^{G12C} inhibitor or a KRAS^{G12C} degrader, (ii) a tri-complex KRAS^{G12D} inhibitor or a KRAS^{G12D} degrader, (iii) a tri-complex KRAS^{G12R} inhibitor or a KRAS^{G12R} degrader, or (iv) a tri-complex KRAS^{G12S} inhibitor or a KRAS^{G12S} degrader.

In some embodiments, the peptide comprises the KRAS^{G12C} mutation, and the targeted covalent inhibitor is a KRAS^{G12C} inhibitor. In some embodiments, the peptide comprises the KRAS^{G12D} mutation, and the targeted covalent inhibitor is a KRAS^{G12D} inhibitor. In some embodiments, the peptide comprises the KRAS^{G12R} mutation, and the targeted covalent inhibitor is a KRAS^{G12R} inhibitor. In some embodiments, the peptide comprises the KRAS^{G12S} mutation, and the targeted covalent inhibitor is a KRAS^{G12S} inhibitor.

In some embodiments, the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

In some embodiments, peptide comprises a segment of EGFR.

In some embodiments, the peptide comprises the amino acid sequence of

QLMPFGCLL, LMPFGCLLDY, or MPFGCLLDY.

In some embodiments, the peptide comprises a segment of EGFR and the targeted covalent inhibitor is an inhibitor of an EGFR family kinase.

In some embodiments, the targeted covalent inhibitor is osimertinib or neratinib.

In some embodiments, peptide comprises a segment of BTK.

In some embodiments, the peptide comprises the amino acid sequence of

YMANGCLLNY.

In some embodiments, the targeted covalent inhibitor is ibrutinib.

In some embodiments, the peptide is a full length protein that is processed in the cell after the covalent reaction with the targeted covalent inhibitor, such that smaller peptide fragments are produced.

In some embodiments, the peptide conjugate comprises a compound selected from the group consisting of compounds 1-8:

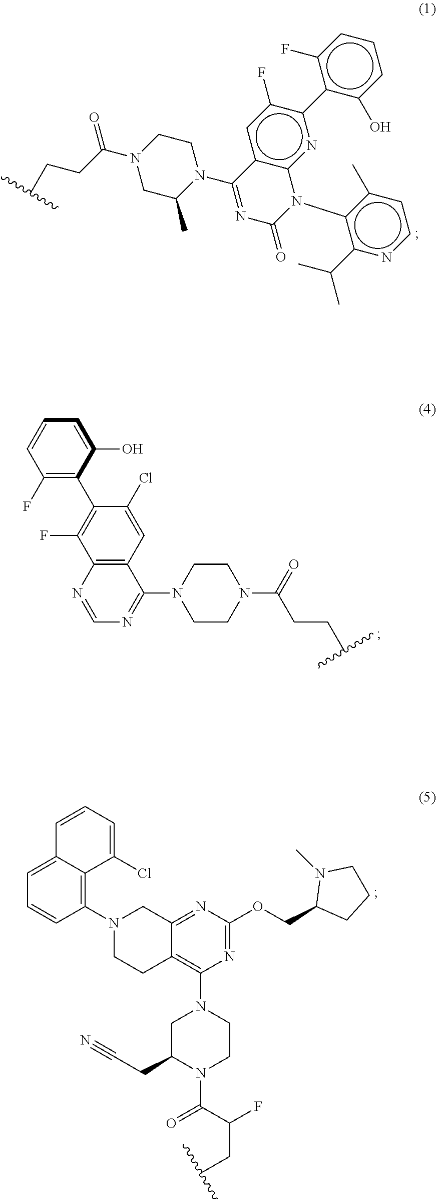

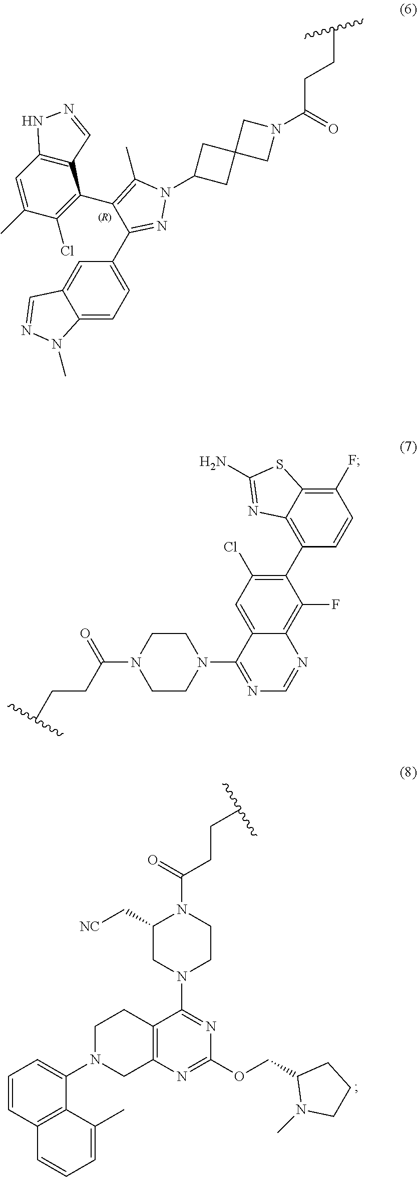

covalently bonded to the cysteine residue in a peptide comprising the amino acid sequence of

VVVGACGVGK, VVGACGVGK, or KLVVVGACGV.

In some embodiments, the MHC is HLA-A*02:01, HLA-A*03:01, and/or HLA-A*11:01.

In some embodiments, the peptide comprises the amino acid sequence of VVVGACGVGK or VVGACGVGK and the MHC is HLA-A*03:01 or HLA-A*11:01. In some embodiments, the peptide comprises the amino acid sequence of KLVVVGACGV and the MHC is HLA-A*02:01.

In some embodiments, the peptide conjugate comprises compound 9:

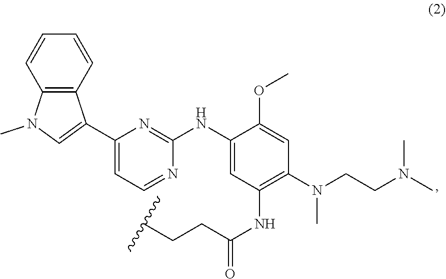

covalently bonded to the cysteine residue in a peptide comprising the amino acid sequence of QLMPFGCLL, LMPFGCLLDY, or MPFGCLLDY.

In some embodiments, the MHC is HLA-A*02, HLA-A*01, HLA-A*03, or HLA-A*26.

In some embodiments, the peptide conjugate comprises compound 10:

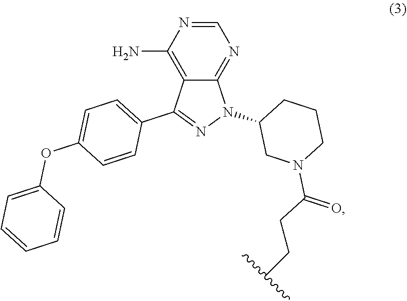 [SMILES: *CCC(=O)N1CCC[C@@H](n2nc(-c3ccc(Oc4ccccc4)cc3)c3c(N)ncnc32)C1]

covalently bonded to the cysteine residue in a peptide comprising the amino acid sequence of YMANGCLLNY.

In some embodiments, the MHC is HLA-A*01:01.

In some embodiments, the binding partner specifically binds to a peptide conjugate/MHC complex, wherein the peptide conjugate is formed by the covalent reaction of sotorasib with a KRAS^{G12C} peptide, wherein the binding partner comprises a heavy chain variable region (VH) and a light chain variable region (VL). In some embodiments, the VH comprises: (i) a CDR-H1 comprising the amino acid sequence of DYSIH, or a variant thereof comprising 1-3 amino acid changes; (ii) a CDR-H2 comprising the amino acid sequence of SISSSSGSTSYADSVKG, or a variant thereof comprising 1-5 amino acid changes; and/or (iii) a CDR-H3 comprising the amino acid sequence of GX₁WX₂X₃AMDY, wherein X₁ is G, R, H, S, or K, X₂ is Y or I, and X₃ is P or A. In some embodiments, the VL comprises: (i) a CDR-L1 comprising the amino acid sequence of RASQSVSSAVA, or a variant thereof comprising 1-5 amino acid changes; (ii) a CDR-L2 comprising the amino acid sequence of SASSLYS, or a variant thereof comprising 1-5 amino acid changes, and/or (iii) a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

In some embodiments, the binding partner specifically binds to a peptide conjugate/MHC complex, wherein the peptide conjugate is formed by the covalent reaction of sotorasib with a KRAS^{G12C} peptide, wherein the binding partner comprises the CDR-H1, CDR-H2, and CDR-H3 amino acid sequences of a VH amino acid sequence and/or the CDR-L1, CDR-L2, and CDR-L3 amino acid sequences of a VL amino acid sequence of a binding partner selected from the group consisting of RA_D11, RA_D01-RA_D04, RA_D06-RA_D09, RA_D12-RA_D14, RA_D16, RA_D18-RA_D21, RA_D23, and RA_D24; or a variant thereof comprising 1-5 amino acid changes in one or more of the CDR amino acid sequences.

In some embodiments, the binding partner comprises the CDR-H1, CDR-H2, CDR-H3, CDR-L1, CDR-L2, and/or CDR-L3 amino acid sequences of a binding partner selected from the group consisting of RA_D11, RA_D01-RA_D04, RA_D06-RA_D09, RA_D12-RA_D14, RA_D16, RA_D18-RA_D21, RA_D23, and RA_D24 (Tables G and H), or a variant thereof comprising 1-5 amino acid changes in one or more of the CDR amino acid sequences.

In some embodiments, the binding partner comprises a VH and/or a VL amino acid sequence that is 90%, 95%, or 100% identical to the following VH and VL sequences:

RA_D11
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRKTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGGWIAAMDYWGQGTLVTVSS
RA_D01
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRRTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGGWIAAMDYWGQGTLVTVSS
RAD02
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQPSYVRRKITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D03
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVARKITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGGWIAAMDYWGQGTLVTVSS
RA_D04
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQVSYVARRITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D06
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVKRLITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGHWIAAMDYWGQGTLVTVSS
RA_D07
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRRLITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D08
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRRVITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGGWIAAMDYWGQGTLVTVSS
RA_D09
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRRTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGSWIAAMDYWGQGTLVTVSS
RA_D12
V_{L}: DIQMTQSPSSLSASVGDRVTITCRASQSVSSAVAWYQQKPGKAPKLLIYSASSLYSGVPSRFSG
SRSGTDTLTISSLQPEDFATYYCQQASYVAKTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D13
V_{L}: DIQMTQSPSSLSASVGDRVTITCRAGQSVSSAVAWYQQKPGKAPKLLIYSASSLYSGVPSRFSG
SRSGTDFTLTISSLQPEDFATYYCQQSSYVRRKITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGKWIPAMDYWGQGTLVTVSS
RA_D14
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRKAITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGGWIAAMDYWGQGTLVTVSS
RA_D16
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRRAITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D18
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQSSYVKRTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D19
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQPSYVRKTITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D20
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVKKEITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D21
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQSSYVHKLITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS
RA_D23
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVRREITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGHWIAAMDYWGQGTLVTVSS
RA_D24
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQASYVHRLITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS

In some embodiments, the binding partner specifically binds to a peptide conjugate/MHC complex, wherein the peptide conjugate is formed by the covalent reaction of osimertinib with an EGFR peptide, wherein the binding partner comprises a heavy chain variable region (VH) and a light chain variable region (VL). In some embodiments, the VH comprises: (i) a CDR-H1 comprising the amino acid sequence of SSYIH, or a variant thereof comprising 1-5 amino acid changes; (ii) a CDR-H2 comprising the amino acid sequence of YISPSYGSTSYADSVKG, or a variant thereof comprising 1-5 amino acid changes; and/or (iii) a CDR-H3 comprising the amino acid sequence of EX₁X₂X₃MX₄X₅DY, wherein X₁ is Y, L, S, or E, X₂ is V, T, or I, X₃ is T or I, X₄ is A, T, or S, and X₅ is L, A, I, K, P, or T. In some embodiments, the VL comprises: (i) a CDR-L1 comprising the amino acid sequence of RASQSVSSAVA, or a variant thereof comprising 1-5 amino acid changes; (ii) a CDR-L2 comprising the amino acid sequence of SASSLYS, or a variant thereof comprising 1-5 amino acid changes; and/or (iii) a CDR-L3 comprising the amino acid sequence of QQYX₁X₂WPX₃T, wherein X₁ is S or A, or S; X₂ is Y, H, A, D, E, K, S, or G; and X₃ is I or E.

In some embodiments, the binding partner specifically binds to a peptide conjugate/MHC complex, wherein the peptide conjugate is formed by the covalent reaction of osimertinib with an EGFR peptide, wherein the binding partner comprises the CDR-H1, CDR-H2, and CDR-H3 amino acid sequences of a VH amino acid sequence and/or the CDR-L1, CDR-L2, and CDR-L3 amino acid sequences of a VL amino acid sequence of a binding partner selected from the group consisting of OEA2-5, EO_Q01-EO_Q18, and EO_Q20-EO_Q24, or a variant thereof comprising 1-5 amino acid changes in one or more of the CDR amino acid sequences.

In some embodiments, the binding partner comprises the CDR-H1, CDR-H2, CDR-H3, CDR-L1, CDR-L2, and/or CDR-L3 amino acid sequences of a binding partner selected from the group consisting of OEA2-5, EO_Q01-EO_Q18, and EO_Q20-EO_Q24 (Tables I and J), or a variant thereof comprising 1-5 amino acid changes in one or more of the CDR amino acid sequences.

In some embodiments, the binding partner comprises a VH and/or a VL amino acid sequence that is 90%, 95%, or 100% identical to the following VH and VL sequences:

EO_Q01
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELVTMTADYWGQGTLVTVSS
EO_Q02
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSAWPETFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYTTMSIDYWGQGTLVTVSS
EO_Q03
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSAWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARESVTMSADYWGQGTLVTVSS
EO_Q04
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARESVTMTKDYWGQGTLVTVSS
EO_Q05
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYAEWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREEVTMSIDYWGQGTLVTVSS
EO_Q06
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARESITMTKDYWGQGTLVTVSS
EO_Q07
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYAKWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELTTMSIDYWGQGTLVTVSS
EO_Q08
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELTEMTTDYWGQGTLVTVSS
EO_Q09
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREEVTMTADYWGQGTLVTVSS
EO_Q10
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSGWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELIEMTPDYWGQGTLVTVSS
EO_Q11
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSGWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELVTMTIDYWGQGTLVTVSS
EO_Q12
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYASWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELTTMSIDYWGQGTLVTVSS
EO_Q13
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELTTMTADYWGQGTLVTVSS
EO_Q14
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARESITMSPDYWGQGTLVTVSS
EO_Q15
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELIEMTTDYWGQGTLVTVSS
EO_Q16
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYVTMSIDYWGQGTLVTVSS
EO_Q17
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCARELVEMTPDYWGQGTLVTVSS
EO_Q18
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSAWPETFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYVTMALDYWGQGTLVTVSS
EO_Q20
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREEVTMTPDYWGQGTLVTVSS
EO_Q22
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYVTMALDYWGQGTLVTVSS
EO_Q23
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSHWPETFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYVTMALDYWGQGTLVTVSS
EO_Q24
V_{L}: DIQMTQSPSSLSASVGDRVTITCRASQSVSSAVAWYQQKPGKAPKLLIYSASSLYSGVPSRFSG
SRSGTDFTLTISSLQPEDFATYYCQQYSEWPETFGQGTKVEIKRTV
V_{H}: EVQLVESGGGLVQPGGSLRLSCAASGFTISSSYIHWVRQAPGKGLEWVAYISPSYGSTSYADS
VKGRFTISADTSKNTAYLQMNSLRAEDTAVYYCAREYVTMALDYWGQGTLVTVSS

In some embodiments, the binding partner has a higher affinity for the peptide conjugate/MHC complex comprising a first HLA than for the peptide conjugate/MHC complex comprising a second HLA.

In some embodiments, the first and second HLA molecules are each selected from the group consisting of HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, and/or HLA-B*15:01.

In some embodiments, the binding partner is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BITE), bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer. In some embodiments, the binding partner is bispecific. In some embodiments, the binding partner specifically binds to the peptide conjugate/MHC complex and a T cell antigen. In some embodiments, the binding partner specifically binds to the peptide conjugate/MHC complex and human CD3.

In some embodiments, the binding partner comprises the VH and/or VL amino acid sequences of

UCHT1:
V_{H}: EVQLQQSGPELVKPGASMKISCKASGYSFTGYTMNWVKQSHGKNL
EWMGLINPYKGVSTYNQKFKDKATLTVDKSSSTAYMELLSLTSEDSAVY
YCARSGYYGDSDWYFDVWGQGTTLTVSS
V_{L}: DIQMTQTTSSLSASLGDRVTISCRASQDIRNYLNWYQQKPDGTVK
LLIYYTSRLHSGVPSKFSGSGSGTDYSLTISNLEQEDIATYFCQQGNTL
PWTFAGGTKLEIK.

In some embodiments, the binding partner comprises a sequence that is at least 90% similar to any of the following sequences, excluding underlined sequences:

OEA2-5_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQYSYWPITFGQGTKVEIKGGGGSEVQLQQSGPELVKPGA
SMKISCKASGYSFTGYTMNWVKQSHGKNLEWMGLINPYKGVSTYNQKFKDKATLTVDKSSSTA
YMELLSLTSEDSAVYYCARSGYYGDSDWYFDVWGQGTTLTVSSGGGGSGGGGSGGGGSDIQMT
QTTSSLSASLGDRVTISCRASQDIRNYLNWYQQKPDGTVKLLIYYTSRLHSGVPSKFSGSGSGTDY
SLTISNLEQEDIATYFCQQGNTLPWTFAGGTKLEIKGGGGSEVQLVESGGGLVQPGGSLRLSCAAS
GFTISSSYIHWVRQAPGKGLEWVAYISPSYGSTSYADSVKGRFTISADTSKNTAYLQMNSLRAEDT
AVYYCAREYVTMALDYWGQGTLVTVSSLEGGGGLNDIFEAQKIEWHESRHHHHHH
EO_Q16_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQYSDWPITFGQGTKVEIKGGGGSEVQLQQSGPELVKPGA
SMKISCKASGYSFTGYTMNWVKQSHGKNLEWMGLINPYKGVSTYNQKFKDKATLTVDKSSSTA
YMELLSLTSEDSAVYYCARSGYYGDSDWYFDVWGQGTTLTVSSGGGGSGGGGSGGGGSDIQMT
QTTSSLSASLGDRVTISCRASQDIRNYLNWYQQKPDGTVKLLIYYTSRLHSGVPSKFSGSGSGTDY
SLTISNLEQEDIATYFCQQGNTLPWTFAGGTKLEIKGGGGSEVQLVESGGGLVQPGGSLRLSCAAS
GFTISSSYIHWVRQAPGKGLEWVAYISPSYGSTSYADSVKGRFTISADTSKNTAYLQMNSLRAEDT
AVYYCAREYVTMSIDYWGQGTLVTVSSLEGGGGLNDIFEAQKIEWHESRHHHHHH
EO_Q17_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQYSSWPITFGQGTKVEIKGGGGSEVQLQQSGPELVKPGAS
MKISCKASGYSFTGYTMNWVKQSHGKNLEWMGLINPYKGVSTYNQKFKDKATLTVDKSSSTAY
MELLSLTSEDSAVYYCARSGYYGDSDWYFDVWGQGTTLTVSSGGGGSGGGGSGGGGSDIQMTQ
TTSSLSASLGDRVTISCRASQDIRNYLNWYQQKPDGTVKLLIYYTSRLHSGVPSKFSGSGSGTDYS
LTISNLEQEDIATYFCQQGNTLPWTFAGGTKLEIKGGGGSEVQLVESGGGLVQPGGSLRLSCAASG
FTISSSYIHWVRQAPGKGLEWVAYISPSYGSTSYADSVKGRFTISADTSKNTAYLQMNSLRAEDTA
VYYCARELVEMTPDYWGQGTLVTVSSLEGGGGLNDIFEAQKIEWHESRHHHHHH

In various embodiments, the binding partners described herein do not comprise the amino acid sequence LEGGGGLNDIFEAQKIEWHESRHHHHHH. In various embodiments, the binding partners described herein do not comprise an AviTag or a His tag.

In various embodiments, the binding partners to be administered into a subject do not comprise the amino acid sequence LEGGGGLNDIFEAQKIEWHESRHHHHHH. In various embodiments, the binding partners to be administered into a subject do not comprise an AviTag or a His tag.

In some embodiments, the binding partner is a single-chain Diabody (scDb) and comprises a sequence that is at least 90% identical to any of the following sequences, excluding underlined sequences:

RA_D01_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQASYVRRTITFGQGTKVEIKGGGGSEVQLQQSGPELVKP
GASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVSSGGGGSGGGGSGGGGSDIQ
MTQTTSSLSASLGDRVTISCRASQDIRNYLNWYQQKPDGTVKLLIYYTSRLHSGVPSKFSGSGSGT
DYSLTISNLEQEDIATYFCQQGNTLPWTFAGGTKLEIKGGGGSEVQLVESGGGLVQPGGSLRLSCA
ASGFTFSDYSIHWVRQAPGKGLEWVASISSSSGSTSYADSVKGRFTISADTSKNTAYLQMNSLRAE
DTAVYYCARGGWIAAMDYWGQGTLVTVSSLEGGGGLNDIFEAQKIEWHESRHHHHHH
RA_D08_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQASYVRRVITFGQGTKVEIKGGGGSEVQLQQSGPELVKP
GASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVSSGGGGSGGGGSGGGGSDIQ
MTQTTSSLSASLGDRVTISCRASQDIRNYLNWYQQKPDGTVKLLIYYTSRLHSGVPSKFSGSGSGT
DYSLTISNLEQEDIATYFCQQGNTLPWTFAGGTKLEIKGGGGSEVQLVESGGGLVQPGGSLRLSCA
ASGFTFSDYSIHWVRQAPGKGLEWVASISSSSGSTSYADSVKGRFTISADTSKNTAYLQMNSLRAE
DTAVYYCARGGWIAAMDYWGQGTLVTVSSLEGGGGLNDIFEAQKIEWHESRHHHHHH
RA_D11_UCHT1_scDb
DIVRSDIQMTQSPSSLSASVGDRVTITCRASQSVSSAVAWYQQKPGKAPKLLIYSASSLYSGVPSRF
SGSRSGTDFTLTISSLQPEDFATYYCQQASYVRKTITFGQGTKVEIKGGGGSEVQLQQSGPELVKP
GASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVSSGGGGSGGGGSGGGGSDIQ
MTQTTSSLSASLGDRVTISCRASQDIRNYLNWYQQKPDGTVKLLIYYTSRLHSGVPSKFSGSGSGT
DYSLTISNLEQEDIATYFCQQGNTLPWTFAGGTKLEIKGGGGSEVQLVESGGGLVQPGGSLRLSCA
ASGFTFSDYSIHWVRQAPGKGLEWVASISSSSGSTSYADSVKGRFTISADTSKNTAYLQMNSLRAE
DTAVYYCARGGWIAAMDYWGQGTLVTVSSLEGGGGLNDIFEAQKIEWHESRHHHHHH

In some embodiments, the binding partner comprises a heavy chain constant region selected from the group consisting of human IgM, IgG₁, IgG₂, IgG₃, IgG₄, IgA₁, and IgA₂.

In some embodiments, the heavy chain constant region comprises one or more amino acid substitutions in the Fc region.

In some embodiments, the binding partner comprises a human kappa light chain constant region or a human lambda light chain constant region.

In some embodiments, the binding partner is in a CrossMab format.

In some embodiments, the binding partner is conjugated to a detectable label, a chemotherapeutic agent, a radioisotope, or a toxin.

In some embodiments, the binding partner is comprised within a chimeric antigen receptor.

In some embodiments, the binding partner is expressed by a T cell, macrophage, neutrophil, or natural killer cell.

In some embodiments, binding of the binding partner to the peptide conjugate/MHC complex is not inhibited by free targeted covalent inhibitor.

In another aspect, the present disclosure provides a complex comprising a binding partner disclosed herein and the peptide conjugate/MHC complex.

In another aspect, the present disclosure provides a polynucleotide encoding the binding partner of disclosed herein.

In another aspect, the present disclosure provides a polynucleotide encoding a heavy chain variable region and/or a light chain variable region of the binding partner disclosed herein.

In another aspect, the present disclosure provides a vector comprising the polynucleotide disclosed herein.

In some embodiments, the vector is a viral vector. In some embodiments, the viral vector is an adenoviral vector, lentiviral vector, retroviral vector, or adeno-associated viral vector.

In another aspect, the present disclosure provides a recombinant host cell comprising (a) the polynucleotide disclosed herein; (b) the vector disclosed herein; (c) a first polynucleotide encoding a VH or a heavy chain of the binding partner disclosed herein, and a second polynucleotide encoding a VL or a light chain of the binding partner disclosed herein; or (d) a first vector comprising a first polynucleotide encoding a VH or a heavy chain of the binding partner disclosed herein, and a second vector comprising a second polynucleotide encoding a VL or a light chain of the binding partner disclosed herein.

In another aspect, the present disclosure provides a pharmaceutical composition comprising the binding partner disclosed herein, the polynucleotide disclosed herein, the vector disclosed herein, or the host cell disclosed herein and a pharmaceutically acceptable carrier or excipient.

In another aspect, the present disclosure provides a method of producing a binding partner, the method comprising culturing the host cell disclosed herein under suitable conditions so that the polynucleotide is expressed and the binding partner is produced.

In another aspect, the present disclosure provides a eukaryotic cell comprising the polynucleotide disclosed herein or the vector disclosed herein. In some embodiments, the cell is optionally a totipotent, multipotent, or pluripotent stem cell. In some embodiments, the stem cell has an induced stem cell phenotype, or wherein the cell is optionally a leukocyte, optionally a CD4+ T cell, optionally a CD8+ T cell, optionally a γδ T cell, optionally a natural killer cell, a natural killer T cell, mucosal-associated invariant T (MAIT) cell, neutrophil, or a macrophage.

In another aspect, the present disclosure provides a method comprising administering to an individual in need thereof the binding partner disclosed herein, the polynucleotide disclosed herein, the vector disclosed herein, the pharmaceutical composition disclosed herein, or the cell disclosed herein.

In another aspect, the present disclosure provides a method for generating a peptide conjugate/MHC complex, the method comprising contacting a cell with a targeted covalent inhibitor, and isolating the peptide conjugate/MHC complex. In some embodiments, the method comprises identifying the peptide conjugate/MHC complex.

In another aspect, the present disclosure provides a cell free peptide conjugate/MHC complex comprising: (a) an isolated peptide conjugate formed by the covalent reaction of a targeted covalent inhibitor with a peptide; and (b) an MHC.

In some embodiments, the peptide comprises a segment of RAS (e.g., KRAS^{G12C}, KRAS^{G12D}, KRAS^{G12R} KRAS^{G12S}, HRAS^{G12C}, HRAS^{G12D}, HRAS^{G12R}, HRAS^{G12S} NRAS^{G12C} NRAS^{G12D}, NRAS^{G12R} or NRAS^{G12S}), EGFR, BTK, HER2/NEU (ERBB2), HER3 (ERBB3), HER4 (ERBB4), MET (HGFR); FGFR, CDK, Acetylcholine Esterase (ACHE), p90 ribosomal S6 kinase (RSK), TP53, IDH1, GNAS, FBXW7, CTNNB1, DNMT3A, cathepsin B, cathepsin C, cathepsin F, cathepsin H, cathepsin K, cathepsin L, cathepsin O, cathepsin S, cathepsin V, cathepsin W, cathepsin X, a caspase, pancreatic lipase, METAP2, any cancer testis antigen (CTA), a long interspersed element-1 (LINE-1), a short interspersed element that is optionally Alu, or any endogenous retroviral protein.

In some embodiments, the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. In another aspect, the present disclosure provides a cell free peptide conjugate/MHC complex comprising (a) a compound selected from the group consisting of compounds 1 and 4-8,

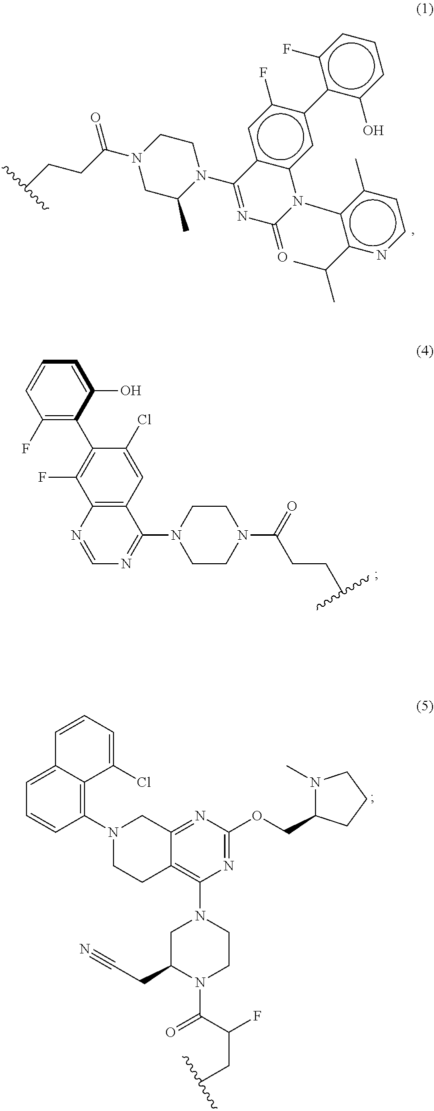

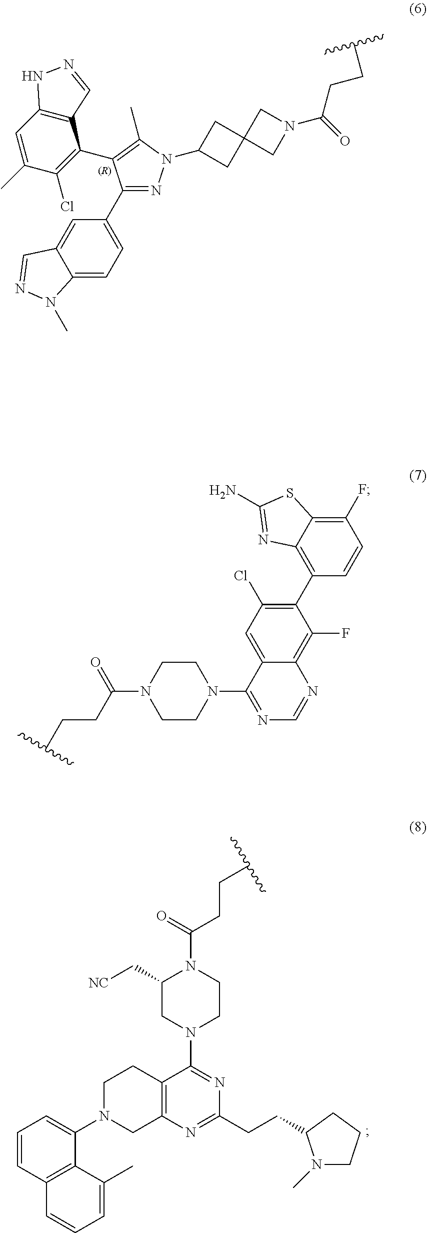

covalently bonded to a cysteine residue in a peptide comprising the amino acid sequence of VVVGACGVGK, VVGACGVGK, or KLVVVGACGV; and (b) an MHC.

In another aspect, the present disclosure provides a cell-free peptide conjugate/MHC complex comprising (a) compound 2:

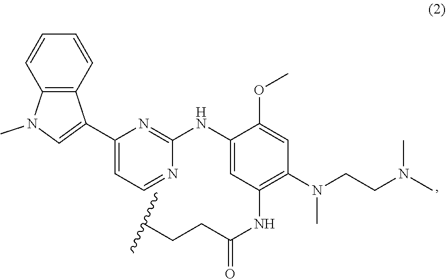

covalently bonded to the cysteine residue in a peptide comprising the amino acid sequence of QLMPFGCLL, LMPFGCLLDY, or MFPGCLLDY; and (b) an MHC.

In another aspect, the present disclosure provides a cell-free peptide conjugate/MHC complex comprising (a) compound 3:

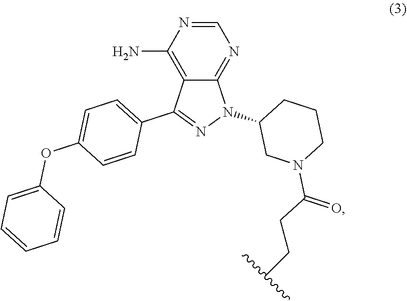 [SMILES: *CCC(=O)N1CCC[C@@H](n2nc(-c3ccc(Oc4ccccc4)cc3)c3c(N)ncnc32)C1]

covalently bonded to the cysteine residue in a peptide comprising the amino acid sequence of YMANGCLLNY; and (b) an MHC.

In some embodiments, the MHC is an HLA, optionally wherein the HLA is an HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, or HLA-B*15:01 molecule.

In another aspect, the present disclosure provides a recombinant cell or particle comprising on its outer surface the cell free peptide conjugate/MHC complex disclosed herein.

In another aspect, the present disclosure provides a method of identifying one or more binding partners that bind with specificity to a peptide conjugate presented by an HLA, the method comprising contacting the cell-free peptide conjugate/MHC complex disclosed herein with a plurality of binding partners, and selecting one or more binding partners that bind with specificity to the cell-free peptide conjugate/MHC complex. In some embodiments, the method further comprises determining the sequence of the one or more selected binding partners. In some embodiments, the method further comprises producing the one or more selected binding partners.

In another aspect, the present disclosure provides a method for identifying a binding partner that specifically binds to a peptide conjugate presented by two or more HLAs, the method comprising providing a sample of cells from a subject who has been treated with a targeted covalent inhibitor and has different HLA types, or providing the cell free peptide conjugate/MHC complex disclosed herein presented by two or more HLAs, and screening binding partners to thereby identify a binding partner that binds with specificity to the peptide conjugate or the antigen that is presented by more than one HLA. In some embodiments, the two or more HLAs types comprise HLA-A*02:01 and at least one additional HLA type. In some embodiments, the two or more HLAs are each HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, or HLA-B*15:01 molecules.

In another aspect, the present disclosure provides a method of killing a cancer cell in a subject, the method comprising administering to the subject: (a) a targeted covalent inhibitor, and (b) the binding partner disclosed herein, the polynucleotide disclosed herein, the vector disclosed herein, the pharmaceutical composition disclosed herein, or the cell disclosed herein.

In some embodiments, the targeted covalent inhibitor targets RAS (e.g., KRAS, HRAS, NRAS), Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, any cancer testis antigen, an endogenous retroviral protein, a long interspersed element-1 (LINE-1), or a short interspersed element (SINE).

In another aspect, the present disclosure provides a method of targeting a cell that expresses EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) in a subject that has been treated with an EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) targeted covalent inhibitor, the method comprising administering to the subject a binding partner disclosed herein, the polynucleotide disclosed herein, the vector disclosed herein, the pharmaceutical composition disclosed herein, or the cell disclosed herein.

In some embodiments, the subject has cancer.

In another aspect, the present disclosure provides a method of treating cancer in a subject that has been treated with a targeted covalent inhibitor, the method comprising administering to the subject a binding partner disclosed herein, the polynucleotide disclosed herein, the vector disclosed herein, the pharmaceutical composition disclosed herein, or the cell disclosed herein.

In another aspect, the present disclosure provides a method of treating a disease or disorder in a subject that has been treated with a targeted covalent inhibitor, the method comprising administering to the subject a binding partner disclosed herein, the polynucleotide disclosed herein, the vector disclosed herein, the pharmaceutical composition disclosed herein, or the cell disclosed herein. In some embodiments, the disease or disorder is an autoimmune disease or a fibrotic disease.

In another aspect, the present disclosure provides a method of enhancing immune recognition of a cell expressing a RAS (e.g., KRAS^{G12C}, KRAS^{G12D}, KRAS^{G12R}, KRAS^{G12S} HRAS^{G12C}, HRAS^{G12D}, HRAS^{G12R}, HRAS^{G12S}, NRAS^{G12C}, NRAS^{G12D}, NRAS^{G12R}, or NRAS^{G12S}) or EGFR mutation in a subject that has a cancer that exhibits a RAS or EGFR mutation, the method comprising administering to the subject: (a) a RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S}, NRAS^{G12C}, NRAS^{G12D}, NRAS^{G12R}, or NRAS^{G12S}) or EGFR inhibitor, and a binding partner disclosed herein, the polynucleotide disclosed herein, the vector disclosed herein, the pharmaceutical composition disclosed herein, or the cell disclosed herein. In some embodiments, the subject has been previously treated with the RAS (e.g., KRAS^{G12C} KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) or EGFR inhibitor.

In some embodiments, the inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. In some embodiments, the targeted covalent inhibitor targets RAS (e.g., KRAS, HRAS, or NRAS), Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, any cancer testis antigen, an endogenous retroviral protein, a long interspersed element-1 (LINE-1), or a short interspersed element (SINE).

In some embodiments, the cancer is renal cell carcinoma, breast cancer, prostate cancer, pancreatic cancer, lung cancer, liver cancer, ovarian cancer, cervical cancer, colon cancer (or colorectal cancer), esophageal cancer, glioma, glioblastoma, brain cancer, stomach cancer, bladder cancer, testicular cancer, head and neck cancer, melanoma, skin cancer, sarcoma, fibrosarcoma, angiosarcoma, osteosarcoma, rhabdomyosarcoma, leukemia, lymphoma, or myeloma.

In some embodiments, the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

In some embodiments, the method disclosed herein further comprises administering an additional therapeutic agent. In some embodiments, the additional therapeutic agent is a chemotherapy, an immunomodulator, or another targeted covalent inhibitor. In some embodiments, the immunomodulator is a checkpoint targeting agent, optionally wherein the checkpoint targeting agent is selected from the group consisting of an antagonist anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, and an agonist anti-CD28 antibody; a cytokine, optionally wherein the cytokine is an anchored IL2 or an engineered IL2; or an inhibitor of extracellular adenosine (eADO) signaling.

In another aspect, the present disclosure provides a method of detecting a peptide conjugate/MHC complex in a biological sample, the method comprising contacting the sample with the binding partner disclosed herein, wherein the peptide conjugate/MHC complex comprises a peptide conjugate formed by the covalent reaction of a targeted covalent inhibitor with a peptide. In some embodiments, the peptide is an EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) peptide. In some embodiments, the targeted covalent inhibitor is an EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D} NRAS^{G12R}, or NRAS^{G12S}) inhibitor. In some embodiments, the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. In some embodiments, the biological sample is blood or serum.

In another aspect, the present disclosure provides a method of identifying a cell containing the peptide conjugate/MHC complex disclosed herein, the method comprising contacting the cell with the binding partner disclosed herein.

In another aspect, the present disclosure provides a method for generating one or more binding partners that specifically bind to a peptide conjugate, wherein said peptide conjugate comprises a peptide covalently bound to a non-peptide molecule, the method comprising: (a) exposing said peptide conjugate to a plurality of binding partners, and b) selecting binding partners that specifically bind to said peptide conjugate to provide one or more selected binding partners. In some embodiments, the method disclosed herein further comprises selecting binding partners that specifically bind to said peptide conjugate but do not detectably bind, or bind with lower affinity, to said peptide or to said non-peptide molecule when they are not covalently bound to each other.

In another aspect, the present disclosure provides a method for generating one or more binding partners that specifically bind to a peptide conjugate presented in the context of a MHC molecule or a fragment or derivative thereof, wherein said peptide conjugate comprises a peptide covalently bound to a non-peptide molecule, the method comprising: (a) providing a complex of said peptide conjugate with said MHC molecule or the fragment or derivative thereof; (b) exposing said complex to a plurality of binding partners, and (c) selecting binding partners that specifically bind to said complex to provide one or more selected binding partners. In some embodiments, the method disclosed herein further comprises selecting binding partners that specifically bind to said complex but do not detectably bind or bind with lower affinity to a complex of said peptide with said MHC molecule or fragment thereof, wherein said peptide is not covalently bound to said non-peptide molecule. In some embodiments, the method disclosed herein further comprises selecting binding partners that specifically bind to said peptide conjugate presented in the context of two or more different MHC molecules.

In some embodiments, the MHC molecule is MHC class I molecule and the peptide is 7-15 amino acids long, or wherein the MHC molecule is a non-classical MHC class I molecule and the peptide is 7-15 amino acids long. In some embodiments, the MHC molecule is MHC class II molecule and the peptide is 9-30 amino acids long.

In some embodiments, the complex is immobilized on a solid support. In some embodiments, the complex is present on a surface of a cell.

In some embodiments, the method further comprises determining whether said one or more binding partners selected in step (c) can mediate immune cell-mediated killing or antibody-dependent cellular cytotoxicity (ADCC), antibody dependent cellular phagocytosis (ADCP), or complement dependent cytotoxicity (CDC)-mediated killing of said cell, or by use of said binding partners as immunotoxins or as antibody-drug conjugates (ADCs) or as radioconjugates. In some embodiments, the plurality of binding partners is generated by a phage-display library or yeast-display library.

In another aspect, the present disclosure provides a method of killing a cancer cell, the method comprising administering to said cell a binding partner that specifically binds to a peptide conjugate, wherein said peptide conjugate comprises (i) a peptide derived from a target protein within said cell which peptide is covalently bound to (ii) a non-peptide molecule, and wherein said binding partner mediates an immune cell-mediated killing or antibody-drug conjugate (ADC)-mediated, antibody dependent cellular phagocytosis (ADCP), or complement dependent cytotoxicity (CDC) immunotoxin killing of said cell, or by using the binding partner as an immunotoxin, or by action of a radioconjugate, to thereby kill the cancer cell.

In some embodiments, the peptide conjugate is presented on said cell in the context of an MHC molecule. In some embodiments, the binding partner specifically binds to a complex comprising said peptide conjugate and said MHC molecule. In some embodiments, the binding partner does not detectably bind to a complex of said peptide with said MHC molecule, wherein said peptide is not covalently bound to said non-peptide molecule.

In some embodiments, the method disclosed herein further comprises administering to said cell said non-peptide molecule, wherein said non-peptide molecule forms a covalent bond with said target protein in said cell. In some embodiments, the non-peptide molecule is administered to said cell prior to administering said binding partner. In some embodiments, the cell is isolated from a subject. In some embodiments, the cell is in a subject and said binding partner is administered to the subject. In some embodiments, the cell is in a subject and said non-peptide molecule and said binding partner are administered to the subject. In some embodiments, the target protein is alternatively spliced or over-expressed in cancer cells but is not alternatively spliced or over-expressed by cancer cells. In some embodiments, the target protein is encoded by a gene that is mutated in cancer cells but not mutated in non-cancer cells. In some embodiments, the non-peptide molecule is a covalent inhibitor of said target protein.

In some embodiments, binding partner is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a Diabody (Db), a Dual-Affinity Retargeting (DART) molecule, a single-chain variable fragment (scFv), a bispecific T-cell engager (BiTE), a bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer. In some embodiments, the CAR is present on a T cell, natural killer (NK) cell, neutrophil, or macrophage. In some embodiments, the binding partner is an antibody-drug conjugate (ADC), a radioconjugate, or toxin conjugate.

In some embodiments, the target protein is selected from RAS (e.g., KRAS, HRAS, or NRAS), Bruton's tyrosine kinase (BTK), a member of epidermal growth factor receptor (EGFR) family, a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, pancreatic lipase, METAP2, a Cancer Testis Antigen, viral polymerase, a protein required for viral cell entry, a protein encoded by a transposable element (e.g. an endogenous retrovirus), or a mutant thereof. In some embodiments, the target protein is a KRAS protein comprising G12C mutation and said non-peptide molecule is selected from sotorasib, ARS-853, ARS-1620, ARS-3248, MRTX849, JNJ74699157, LY3499446, LY3537982, MRTX-1257, JDQ443, RMC-6291, DC-6036, D-1553, 2E07, 6H05, SML-8-73-1, or BI 182391, and derivatives thereof. In some embodiments, the target protein is EGFR and said non-peptide molecule is selected from PD168393, PF00299804 (dacomitinib), EKB569 (pelitinib), afatinib, WZ4002, osimertinib (AZD9291), PF-06459988, nazartinib, naquotinib, olmutinib, avitinib, rociletinib, neratinib, pyrotinib, poziotinib, and derivatives thereof. In some embodiments, the target protein is Bruton's tyrosine kinase (BTK) and said non-peptide molecule is selected from ibrutinib, acalabrutinib, zanubrutinib, CHMFL-BTK-11, ONO/GS-405, PRN1008, CC-292, and derivatives thereof. In some embodiments, the target protein is p90 ribosomal S6 kinase (RSK) and said non-peptide molecule is fluoromethylketone (FMK), dimethyl fumarate, or derivatives thereof. In some embodiments, the target protein is FGFR and said non-peptide molecule is selected from FIIN-1, FIIN-2, FIIN-3, BGJ398, AZD4547, PRN1371, FGF401, and derivatives thereof.

In another aspect, the present disclosure provides a method of treating a cancer in a subject in need thereof, the method comprising administering to said subject an effective amount of a binding partner that specifically binds to a peptide conjugate, wherein said peptide conjugate comprises (i) a peptide derived from a target protein present in cancer cells of said subject, which peptide is covalently bound to (ii) a non-peptide molecule, and wherein said binding partner mediates immune cell-mediated killing or antibody-drug conjugate (ADC)-mediated killing of cancer cells, antibody dependent cellular phagocytosis (ADCP), or complement dependent cytotoxicity (CDC)-mediated killing of said cancer cell, or by use of said binding partners as immunotoxins or radioconjugates in said subject, to thereby kill the cancer cell. In some embodiments, the peptide conjugate is presented on cancer cells of said subject in the context of an MHC molecule. In some embodiments, the binding partner specifically binds to a complex comprising said peptide conjugate and said MHC molecule. In some embodiments, the binding partner does not detectably bind to a complex of said peptide with said MHC molecule, wherein said peptide is not covalently bound to said non-peptide molecule. In some embodiments, the subject has previously received said non-peptide molecule. In some embodiments, the method disclosed herein further comprises administering to said subject an effective amount of said non-peptide molecule, wherein said non-peptide molecule forms a covalent bond with said target protein in cancer cells of said subject. In some embodiments, the non-peptide molecule is administered to said subject prior to administering said binding partner.

In another aspect, the present disclosure provides a method for improving efficacy of an anti-cancer treatment in a subject in need thereof, wherein said anti-cancer treatment comprises administering to said subject a non-peptide molecule, which non-peptide molecule forms a covalent bond with a target protein in cancer cells of said subject, the method comprising further administering to said subject an effective amount of a binding partner that specifically binds to a peptide conjugate, wherein said peptide conjugate comprises a peptide derived from said target protein covalently bound to said non-peptide molecule, and wherein said binding partner mediates immune cell-mediated killing or antibody-drug conjugate (ADC)-mediated killing of cancer cells, antibody dependent cellular phagocytosis (ADCP), or complement dependent cytotoxicity (CDC) mediated killing of said cancer cell, or by use of said binding partners as immunotoxins, or by use of said binding partners as radioconjugates, in said subject. In some embodiments, the peptide conjugate is presented on cancer cells of said subject in the context of an MHC molecule. In some embodiments, the binding partner specifically binds to a complex comprising said peptide conjugate and said MHC molecule. In some embodiments, the binding partner does not detectably bind to a complex of said peptide with said MHC molecule, wherein said peptide is not covalently bound to said non-peptide molecule. In some embodiments, the subject has previously received said non-peptide molecule prior to administering said binding partner.

In another aspect, the present disclosure provides a kit comprising (i) a non-peptide molecule, wherein said non-peptide molecule forms a covalent bond with a target protein in a cell, (ii) a binding partner that specifically binds to a peptide conjugate, wherein said peptide conjugate comprises a peptide derived from said target protein covalently bound to said non-peptide molecule, and optionally (iii) instructions for use.

In another aspect, the present disclosure provides an isolated peptide conjugate comprising a peptide of 7-30 amino acids in length comprising an amino acid sequence that is at least 80% identical to the amino acid sequence VVGACGVGK, wherein the peptide is conjugated to sotorasib or a derivative thereof.

In another aspect, the present disclosure provides an isolated peptide conjugate comprising a peptide of 7-30 amino acids in length comprising an amino acid sequence that is at least 80% identical to the amino acid sequence QLMPFGCLL, wherein the peptide is conjugated to osimertinib or a derivative thereof.

In another aspect, the present disclosure provides an isolated peptide conjugate comprising a peptide of 7-30 amino acids in length comprising an amino acid sequence that is at least 80% identical to the amino acid sequence YMANGCLLNY, wherein the peptide is conjugated to ibrutinib or a derivative thereof.

In another aspect, the present disclosure provides an isolated molecular complex comprising the peptide conjugate disclosed herein and an MHC molecule, or a fragment or derivative thereof.

In another aspect, the present disclosure provides a host cell comprising the molecular complex disclosed herein.

In another aspect, the present disclosure provides a solid surface carrier comprising the molecular complex disclosed herein.

In another aspect, the present disclosure provides a kit comprising (i) the peptide conjugate disclosed herein, the molecular complex disclosed herein, the cell disclosed herein, the solid surface carrier of disclosed herein, or any combination thereof, and optionally (ii) instructions for use.

In another aspect, the present disclosure provides an isolated binding partner that specifically binds the peptide conjugate disclosed herein or the molecular complex disclosed herein. In some embodiments, the binding partner is an antibody or an antigen-binding fragment thereof. In some embodiments, the binding partner is a component of a chimeric antigen receptor (CAR).

In another aspect, the present disclosure provides a fusion protein comprising the binding partner disclosed herein, said fusion protein comprising (i) at least one cytokine, and/or (ii) an additional antibody or fragment thereof that is an immune checkpoint inhibitor. In some embodiments, the cytokine is selected from the group consisting of interleukin (IL)-2, IL-7, IL-8, IL-15, IL-17, IL-18, and a combination thereof, and/or (ii) the additional antibody or fragment thereof that is an immune checkpoint inhibitor is an anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody. In some embodiments, the binding partner comprises a component that binds to a T cell or natural killer (NK) cell protein, wherein said T cell or NK cell protein is selected from the group consisting of a T cell receptor protein, CD4, CD8, CD28, CD16A, NKG2D, NK_{D} 30, NK_{D} 46, and a combination thereof.

In another aspect, the present disclosure provides a polypeptide comprising an antigen-binding domain comprising a heavy chain variable region (VH) and/or a light chain variable region (VL). In some embodiments, the VH comprises a heavy chain complementarity determining region 3 (CDR-H3) comprising the amino acid sequence of GX₁WX₂X₃AMDY, wherein X₁ is G, R, H, S, or K, X₂ is Y or I, and X₃ is P or A. In some embodiments, the VL comprises a light chain complementarity determining region 3 (CDR-L3) comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide binds to an epitope of the MHC.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the antigen-binding domain binds to the peptide conjugate/MHC complex with a dissociation constant (K_{D}) of at most about 50 nM.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the antigen-binding domain binds to the free targeted covalent inhibitor with a dissociation constant (K_{D}) of at least 200 nM.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein: (a) the polypeptide binds to the peptide conjugate/MHC complex at an angle from about 10° to 60° between the axis of the MHC and the axis of the polypeptide; (b) when the polypeptide is bound to the peptide conjugate/MHC complex, the angle between the axis of the polypeptide and the axis of the peptide conjugate/MHC complex is from about 10° to 60°; (c) when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex; (d) when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex; (e) the polypeptide binds to the peptide conjugate/MHC complex at an angle that is different than the angle to which a T cell receptor binds to the peptide conjugate/MHC complex; and/or (f) the polypeptide binds to the peptide conjugate/MHC complex at an angle that is from about 10° to 60° less or from about 10° to 60° more than the angle to which a T cell receptor binds to the peptide conjugate/MHC complex.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide contacts one or more residues of an alpha 1 domain or region and one or more residues of an alpha 2 domain or region of a heavy chain of the MHC of the peptide conjugate/MHC complex.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide binds to residues 62-66, 106-109, and/or 150-170 of the MHC, or one or more residues thereof.

In another aspect, the present disclosure provides a polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein an interface area of the polypeptide with the peptide conjugate/MHC complex is at least about 500 Å2. In some embodiments, the polypeptide disclosed herein comprises a heavy chain variable region (VH) and/or a light chain variable region (VL). In some embodiments, the VH comprises a CDR-H3 comprising the amino acid sequence of GX₁WX₂X₃AMDY, wherein X₁ is G, R, H, S, or K, X₂ is Y or I, and X₃ is P or A. In some embodiments, the VL comprises a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E. In some embodiments, the VH comprises: (i) a CDR-H1 comprising the amino acid sequence of DYSIH, or a variant thereof comprising 1-3 amino acid changes; (ii) a CDR-H2 comprising the amino acid sequence of SISSSSGSTSYADSVKG, or a variant thereof comprising 1-5 amino acid changes; and/or (iii) a CDR-H3 comprising the amino acid sequence of GX₁WX₂X₃AMDY, wherein X₁ is G, R, H, S, or K, X₂ is Y or I, and X₃ is P or A. In some embodiments, the VL comprises: (i) a CDR-L1 comprising the amino acid sequence of RASQSVSSAVA, or a variant thereof comprising 1-5 amino acid changes; (ii) a CDR-L2 comprising the amino acid sequence of SASSLYS, or a variant thereof comprising 1-5 amino acid changes, and/or (iii) a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

In some embodiments, the VH comprises a CDR-H1 having a sequence of DYSIH, a CDR-H2 having a sequence of SISSSSGSTSYADSVKG, and a CDR-H3 having a sequence of GGWIAAMDY. In some embodiments, the VL comprises a CDR-L1 having a sequence of RASQSVSSAVA, a CDR-L2 having a sequence of SASSLYS, and a CDR-L3 having a sequence of QQASYVRKTIT. In some embodiments, the VH comprises an amino acid sequence having at least about 90%, 95%, or 100% sequence identity to the amino acid sequence EVOLVESGGGLVQPGGSLRLSCAASGFTFSDYSIHWVRQAPGKGLEWVASISSSSGST SYADSVKGRFTISADTSKNTAYLQMNSLRAEDTAVYYCARGGWIAAMDYWGQGTL VTVSS. In some embodiments, the VL comprises an amino acid sequence having at least about 90%, 95%, or 100% sequence identity to the amino acid sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSWIHAMDY. In some embodiments, the VH comprises a CDR-H2 comprising the amino acid sequence of SISSSWGVTSYADSVKG. In some embodiments, the VH comprises a CDR-H1 comprising the amino acid sequence of FHWYSIH. In some embodiments, the antigen-binding domain further comprises a light chain complementarity determining region 3 (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSWIHAMDY, a CDR-H2 sequence of SISSSWGVTSYADSVKG, a CDR-H1 sequence of FHWYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFHWYSIHWVRQAPGKGLEWVASISSSWG VTSYADSVKGRFTISADTSKNTAYLQMNSLRAEDTAVYYCARGSWIHAMDYWGQG TLVTVSS. In some embodiments, the VL comprises a sequence with at least 80% sequence identity to the sequence DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. In some embodiments, the antigen-binding domain comprises a heavy chain variable region (VH), and wherein the VH comprises a heavy chain complementarity determining region 3 (CDR-H3) comprising the amino acid sequence of GHWIAAMDY. In some embodiments, the VH comprises a CDR-H2 comprising the amino acid sequence of SIASSSGSTGYADSVKG. In some embodiments, the VH comprises a CDR-H1 comprising the amino acid sequence of FSWYSIH. In some embodiments, the antigen-binding domain further comprises a light chain variable region (VL), and wherein the VL comprises a light chain complementarity determining region 3 (CDR-L3) comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GHWIAAMDY, a CDR-H2 sequence of SIASSSGSTGYADSVKG, a CDR-H1 sequence of FSWYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSWYSIHWVRQAPGKGLEWVASIASSSGS TGYADSVKGRFTISADTSKNTAYLQMNSLRAEDTAVYYCARGHWIAAMDYWGQG TLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGVIHAMDY. In some embodiments, the VH comprises a CDR-H2 comprising the amino acid sequence of SILSRWGVTSYADSVKG. In some embodiments, the VH comprises a CDR-H1 comprising the amino acid sequence of FSPYSIH. In some embodiments, the antigen-binding domain further comprises a light chain complementarity determining region 3 (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGVIHAMDY, a CDR-H2 sequence of SILSRWGVTSYADSVKG, a CDR-H1 sequence of FSPYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSPYSIHWVRQAPGKGLEWVASILSRWGV TSYADSVKGRFTISADTSKNTAYLQMNSLRAEDTAVYYCARGGVIHAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSWIAAMDY. In some embodiments, the VH comprises a CDR-H2 comprising the amino acid sequence of SISSWHGETGYADSVKG. In some embodiments, the VH comprises a CDR-H1 comprising the amino acid sequence of FSPYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSWIAAMDY, a CDR-H2 sequence of SISSWHGETGYADSVKG, a CDR-H1 sequence of FSPYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSPYSIHWVRQAPGKGLEWVASISSWHGE TGYADSVKGRFTISADTSKNTAYLQMNSLRAEDTAVYYCARGSWIAAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGWIAAMDY. In some embodiments, the VH comprises a CDR-H2 comprising the amino acid sequence of SISSLQGDTGYADSVKG. In some embodiments, the VH comprises a CDR-H1 comprising the amino acid sequence of FSWYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGWIAAMDY, a CDR-H2 sequence of SISSLQGDTGYADSVKG, a CDR-H1 sequence of FSWYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSWYSIHWVRQAPGKGLEWVASISSLQGD TGYADSVKGRFTISADTSKNTAYLQMNSLRAEDTAVYYCARGGWIAAMDYWGQG TLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSWIAAMDY. In some embodiments, the VH comprises a CDR-H2 comprising the amino acid sequence of SIASWYGDTGYADSVKG. In some embodiments, the VH comprises a CDR-H1 comprising the amino acid sequence of FHYYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSWIAAMDY, a CDR-H2 sequence of SIASWYGDTGYADSVKG, a CDR-H1 sequence of FHYYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFHYYSIHWVRQAPGKGLEWVASIASWYG DTGYADSVKGRFTISADTSKNTAYLQMNSLRAEDTAVYYCARGSWIAAMDYWGQG TLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGRIEAMDY. In some embodiments, the VH comprises a CDR-H2 comprising the amino acid sequence of SISSWYGKTGYADSVKG. In some embodiments, the VH comprises a CDR-H1 comprising the amino acid sequence of FGYYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGRIEAMDY, a CDR-H2 sequence of SISSWYGKTGYADSVKG, a CDR-H1 sequence of FGYYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFGYYSIHWVRQAPGKGLEWVASISSWYG KTGYADSVKGRFTISADTSKNTAYLQMNSLRAEDTAVYYCARGGRIEAMDYWGQG TLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GYWIEAMDY. In some embodiments, the VH comprises a CDR-H2 comprising the amino acid sequence of SIASSYGSTGYADSVKG. In some embodiments, the VH comprises a CDR-H1 comprising the amino acid sequence of FSKYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GYWIEAMDY, a CDR-H2 sequence of SIASSYGSTGYADSVKG, a CDR-H1 sequence of FSKYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSKYSIHWVRQAPGKGLEWVASIASSYGS TGYADSVKGRFTISADTSKNTAYLQMNSLRAEDTAVYYCARGYWIEAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSWIAAMDY. In some embodiments, the VH comprises a CDR-H2 comprising the amino acid sequence of SIHSSIGTTGYADSVKG. In some embodiments, the VH comprises a CDR-H1 comprising the amino acid sequence of FGLYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSWIAAMDY, a CDR-H2 sequence of SIHSSIGTTGYADSVKG, a CDR-H1 sequence of FGLYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVQLVESGGGLVQPGGSLRLSCAASGFTFGLYSIHWVRQAPGKGLEWVASIHSSIGT TGYADSVKGRFTISADTSKNTAYLQMNSLRAEDTAVYYCARGSWIAAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSVIHAMDY. In some embodiments, the VH comprises a CDR-H2 comprising the amino acid sequence of SILSWIGKTSYADSVKG. In some embodiments, the VH comprises a CDR-H1 comprising the amino acid sequence of FSPYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSVIHAMDY, a CDR-H2 sequence of SILSWIGKTSYADSVKG, a CDR-H1 sequence of FSPYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSPYSIHWVRQAPGKGLEWVASILSWIGK TSYADSVKGRFTISADTSKNTAYLQMNSLRAEDTAVYYCARGSVIHAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGWIAAMDY. In some embodiments, the VH comprises a CDR-H2 comprising the amino acid sequence of SIASRWGHTGYADSVKG. In some embodiments, the VH comprises a CDR-H1 comprising the amino acid sequence of FSPYHIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGWIAAMDY, a CDR-H2 sequence of SIASRWGHTGYADSVKG, a CDR-H1 sequence of FSPYHIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSPYHIHWVRQAPGKGLEWVASIASRWG HTGYADSVKGRFTISADTSKNTAYLQMNSLRAEDTAVYYCARGGWIAAMDYWGQ GTLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GSWIAAMDY. In some embodiments, the VH comprises a CDR-H2 comprising the amino acid sequence of SIASLQGITGYADSVKG. In some embodiments, the VH comprises a CDR-H1 comprising the amino acid sequence of FHEYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GSWIAAMDY, a CDR-H2 sequence of SIASLQGITGYADSVKG, a CDR-H1 sequence of FHEYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFHEYSIHWVRQAPGKGLEWVASIASLQGI TGYADSVKGRFTISADTSKNTAYLQMNSLRAEDTAVYYCARGSWIAAMDYWGQGT LVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGVIHAMDY. In some embodiments, the VH comprises a CDR-H2 comprising the amino acid sequence of SILSRWGVTSYADSVKG. In some embodiments, the VH comprises a CDR-H1 comprising the amino acid sequence of FSDYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGVIHAMDY, a CDR-H2 sequence of SILSRWGVTSYADSVKG, a CDR-H1 sequence of FSDYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSDYSIHWVRQAPGKGLEWVASILSRWG VTSYADSVKGRFTISADTSKNTAYLQMNSLRAEDTAVYYCARGGVIHAMDYWGQG TLVTVSS. In some embodiments, the VL comprises a sequence with at least 80% sequence identity to the sequence

DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

In some embodiments, the antigen-binding domain comprises a heavy chain variable region (VH), and wherein the VH comprises a heavy chain complementarity determining region 3 (CDR-H3) comprising the amino acid sequence of GGVIHAMDY. In some embodiments, the VH comprises a CDR-H2 comprising the amino acid sequence of SISSRWGVTSYADSVKG. In some embodiments, the VH comprises a CDR-H1 comprising the amino acid sequence of FSDYSIH. In some embodiments, the antigen-binding domain further comprises a light chain variable region (VL), and wherein the VL comprises a CDR-L3 comprising the amino acid sequence of QQASYVRKTIT. In some embodiments, the VL comprises a CDR-L2 comprising the amino acid sequence of SASSLYS. In some embodiments, the VL comprises a CDR-L1 comprising the amino acid sequence of RASQSVSSAVA. In some embodiments, the antigen binding domain comprises: a CDR-H3 sequence of GGVIHAMDY, a CDR-H2 sequence of SISSRWGVTSYADSVKG, a CDR-H1 sequence of FSDYSIH, a CDR-L3 sequence of QQASYVRKTIT, a CDR-L2 sequence of SASSLYS, and a CDR-L1 sequence of RASQSVSSAVA. In some embodiments, the VH comprises a sequence with at least 80% sequence identity to the sequence EVOLVESGGGLVQPGGSLRLSCAASGFTFSDYSIHWVRQAPGKGLEWVASISSRWG VTSYADSVKGRFTISADTSKNTAYLQMNSLRAEDTAVYYCARGGVIHAMDYWGQG TLVTVSS. In some embodiments, the VH comprises a sequence with at least 80% sequence identity to the sequence

EVQLVESGGGLVQPGGSLRLSCAASGFTFSDYSIHWVRQAPGKGLEWVAS
ISSRWGVTSYADSVKGRFTISADTSKNTAYLQMNSLRAEDTAVYYCARGG
VIHAMDYWGQGTLVTVSS.

In some embodiments, the antibody or the antigen-binding fragment specifically binds to a peptide conjugate/MHC complex and wherein the antibody or the antigen-binding fragment interacts with the MHC of the peptide conjugate/MHC complex. In some embodiments, the peptide conjugate/MHC complex comprises: (a) a peptide conjugate comprising a peptide covalently linked to a targeted covalent inhibitor or a fragment thereof; and (b) an MHC. In some embodiments, the MHC is a human leukocyte antigen (HLA). In some embodiments, the HLA is a HLA-A*03:01, HLA-A*11:01, and/or HLA-A*02:01 molecule.

In some embodiments, the antigen-binding domain binds to the peptide conjugate/MHC complex with a greater affinity than to the peptide or free targeted covalent inhibitor. In some embodiments, the antigen-binding domain binds to the peptide conjugate/MHC disclosed herein complex with a dissociation constant (K_{D}) of at most about 50 nM, at most about 40 nM, at most about 30 nM, at most about 20 nM, at most about 10 nM, at most about 1 nM, at most about 0.1 nM, at most about 10 pM, at most about 1 pM, or at most about 0.1 pM. In some embodiments, the antigen-binding domain binds to the peptide conjugate/MHC complex with a dissociation constant (K_{D}) of from about 0.01 nM to about 20 nM. In some embodiments, the antigen-binding domain binds to the free targeted covalent inhibitor with a dissociation constant (K_{D}) of at least about 100 nM, at least about 200 nM, at least about 300 nM, at least about 400 nM, at least about 500 nM, at least about 1 μM, at least about 10 μM, at least about 20 μM, at least about 30 μM, at least about 40 μM, at least about 50 μM, or at least 100 μM. In some embodiments, the antigen-binding domain binds to the free targeted covalent inhibitor with a dissociation constant (K_{D}) of at least about 10, 100, 10,000, or 100,000 times more than a K_{D} of the antibody or the antigen-binding fragment binding to the peptide conjugate/MHC complex. In some embodiments, the antigen-binding domain does not detectably bind to the free targeted covalent inhibitor. In some embodiments, the antigen-binding domain binds to the free targeted covalent inhibitor with an IC₅₀ of at least about 50 nM. In some embodiments, the antigen-binding domain binds to the free peptide conjugate with a dissociation constant (K_{D}) of more than about 100 nM, more than about 200 nM, more than about 300 nM, more than about 400 nM, more than about 500 nM, more than 1 μM, more than 10 μM, more than 20 μM, more than 30 μM, more than 40 μM, more than 50 μM, or more than 100 μM. In some embodiments, the antigen-binding domain binds to the free peptide conjugate with a dissociation constant (K_{D}) that is at least about 10, 100, 10,000, 100,000 times more than a K_{D} of the antibody or the antigen-binding fragment to the peptide conjugate/MHC complex. In some embodiments, the antigen-binding domain binds to the free peptide conjugate with a dissociation constant (K_{D}) that is at least 2.5 times more than a K_{D} of the antibody or the antigen-binding fragment to the peptide conjugate/MHC complex. In some embodiments, the antigen-binding domain binds to the free peptide conjugate with a dissociation constant (K_{D}) that is higher than a K_{D} of the antibody or the antigen-binding fragment to the peptide conjugate/MHC complex, wherein the MHC is HLA-A02:01. In some embodiments, the antigen-binding domain binds to the free peptide conjugate with a dissociation constant (K_{D}) that is at least 2.5 times more than a K_{D} of the antibody or the antigen-binding fragment to the peptide conjugate/MHC complex, wherein the MHC is HLA-A03:01. In some embodiments, the antigen-binding domain binds to the peptide conjugate/MHC complex with an affinity that is at least 100 times, at least 200 times, at least 300 times, at least 400 times, at least 500 times, at least 600 times, at least 700 times, at least 800 times, at least 900 times, at least 1,000 times, at least 2,500 times, at least 5,000 times, or at least 10,000 times greater than the affinity of antibody or the antigen-binding fragment to the free targeted covalent inhibitor or the free peptide conjugate.

In some embodiments, the peptide conjugate is formed by the covalent reaction of a free targeted covalent inhibitor with a KRAS^{G12C} peptide, a KRAS^{G12D} peptide, a KRAS^{G12R} or a KRAS^{G12S} peptide. In some embodiments, the free targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. In some embodiments, the peptide conjugate is formed by the covalent reaction of sotorasib with a KRAS^{G12C} peptide. In some embodiments, the peptide comprises or consists of the amino acid sequence of VVVGACGVGK, VVGACGVGK, or KLVVVGACGV.

In some embodiments, the antigen binding domain (i) has specificity to a peptide conjugate/MHC complex comprising VVGACGVGK conjugated to a targeted covalent inhibitor or fragment thereof presented by HLA-A*11:01 and/or a peptide conjugate/MHC complex comprising VVVGACGVGK conjugated to a targeted covalent inhibitor or fragment thereof presented by HLA-A*11:01, or (ii) has specificity to a peptide conjugate/MHC complex comprising VVGACGVGK conjugated to a targeted covalent inhibitor or fragment thereof presented by HLA-A*03:01 and/or a peptide conjugate/MHC complex comprising VVVGACGVGK conjugated to a targeted covalent inhibitor or fragment thereof presented by HLA-A*03:01. In some embodiments, the antigen binding domain has specificity to a peptide conjugate/MHC complex comprising VVVGACGVGK conjugated to a targeted covalent inhibitor or fragment thereof presented by HLA-A*03:01 and/or a peptide conjugate/MHC complex comprising VVVGACGVGK conjugated to a targeted covalent inhibitor or fragment thereof presented by HLA-A*11:01. In some embodiments, the antigen binding domain has specificity to a peptide conjugate/MHC complex comprising VVGACGVGK conjugated to a targeted covalent inhibitor or fragment thereof presented by HLA-A*11:01, a peptide conjugate/MHC complex comprising VVGACGVGK conjugated to a targeted covalent inhibitor or fragment thereof presented by HLA-A*03:01, a peptide conjugate/MHC complex comprising VVVGACGVGK conjugated to a targeted covalent inhibitor or fragment thereof presented by HLA-A*03:01, a peptide conjugate/MHC complex comprising VVVGACGVGK conjugated to a targeted covalent inhibitor or fragment thereof presented by HLA-A*11:01, and/or a peptide conjugate/MHC complex comprising KLVVVGACGV conjugated to a targeted covalent inhibitor or fragment thereof presented by HLA-A*02:01.

In some embodiments, the polypeptide binds to: (i) a peptide conjugate/HLA-A*11:01 MHC complex containing a peptide consisting of the amino acid sequence VVGACGVGK and a peptide conjugate/HLA-A*11:01 MHC complex containing a peptide consisting of the amino acid sequence VVVGACGVGK; (ii) a peptide conjugate/HLA-A*03:01 MHC complex containing a peptide consisting of the amino acid sequence VVGACGVGK and a peptide conjugate/HLA-A*03:01 MHC complex containing a peptide consisting of the amino acid sequence VVVGACGVGK; (iii) a peptide conjugate/HLA-A*03:01 MHC complex containing a peptide consisting of the amino acid sequence VVGACGVGK and a peptide conjugate/HLA-A*11:01 MHC complex containing a peptide consisting of the amino acid sequence VVGACGVGK; (iv) a peptide conjugate/HLA-A*03:01 MHC complex containing a peptide consisting of the amino acid sequence VVVGACGVGK and a peptide conjugate/HLA-A*11:01 MHC complex containing a peptide consisting of the amino acid sequence VVVGACGVGK; and/or (v) a peptide conjugate/HLA-A*02:01 MHC complex containing a peptide consisting of the amino acid sequence KLVVVGACGV.

In some embodiments, the polypeptide binds to: a peptide conjugate/HLA-A*03:01 MHC complex containing a peptide consisting of the amino acid sequence VVGACGVGK, and a peptide conjugate/HLA-A*03:01 MHC complex containing a peptide consisting of the amino acid sequence VVVGACGVGK, and a peptide conjugate/HLA-A*11:01 MHC complex containing a peptide consisting of the amino acid sequence VVGACGVGK, and a peptide conjugate/HLA-A*11:01 MHC complex containing a peptide consisting of the amino acid sequence VVVGACGVGK, and a peptide conjugate/HLA-A*02:01 MHC complex containing a peptide consisting of the amino acid sequence KLVVVGACGV.

In some embodiments, the targeted covalent inhibitor has a chemical structure comprising C-R1, wherein C is a chemical fragment linked to R1 of any compound selected from the group consisting of

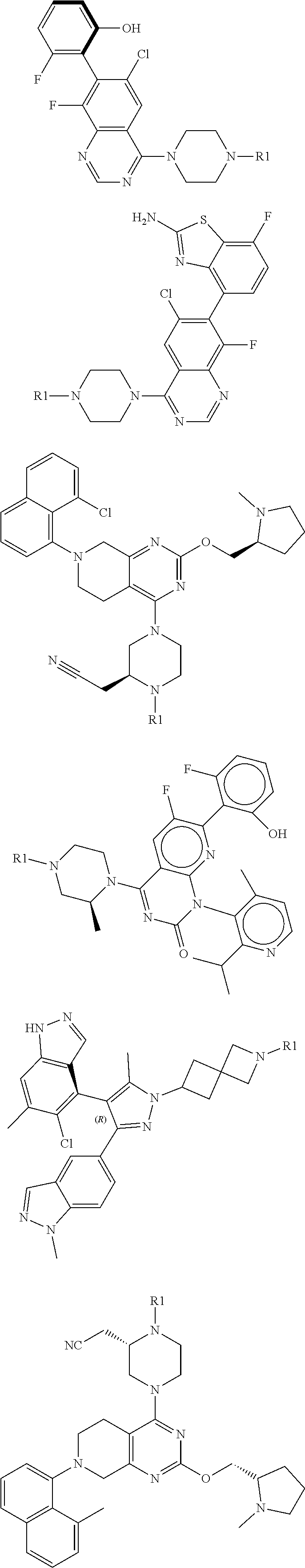

and wherein R1 is selected from

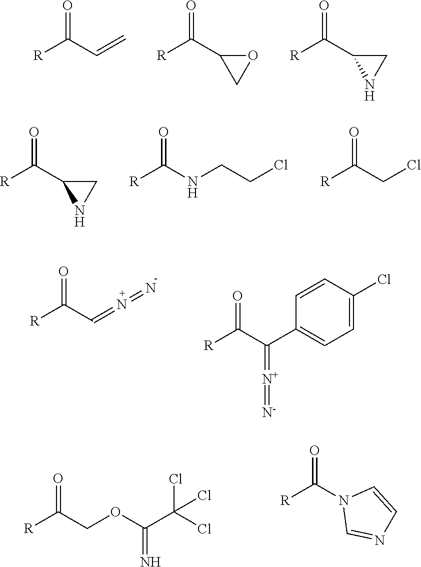

In some embodiments, the targeted covalent inhibitor forms a covalent bond to (i) the cysteine residue in a peptide comprising the amino acid sequence of VVVGACGVGK, VVGACGVGK, or KLVVVGACGV, (ii) the aspartic acid residue in a peptide comprising the amino acid sequence of VVVGADGVGK, VVGADGVGK, or KLVVVGADGV, (iii) the serine acid residue in a peptide comprising the amino acid sequence of VVVGASGVGK, VVGASGVGK, or KLVVVGASGV, or (iv) the arginine residue in a peptide comprising the amino acid sequence of VVVGARGVGK, VVGARGVGK, or KLVVVGARGV.

In some embodiments, the antigen binding domain of the polypeptide recognizes the C portion of the targeted covalent inhibitor of the peptide conjugate/MHC complex.

In some embodiments, the antigen binding domain of the polypeptide recognizes the C portion of the targeted covalent inhibitor but not an R1 portion of the targeted covalent inhibitor of the peptide conjugate/MHC complex.

In some embodiments, an interface area of the polypeptide with the peptide conjugate/MHC complex is at least about 500 Å², 600 Å², 700 Å², 800 Å², 900 Å², 1,000 Å², 1,200 Å², 1,500 Å², or 2,000 Å². In some embodiments, the interface area of the polypeptide with the MHC of the peptide conjugate/MHC complex is more than the interface area of the polypeptide with the peptide or the targeted covalent inhibitor.

In some embodiments, the polypeptide forms a binding pocket at the interface between VH and VL domains to accommodate the targeted covalent inhibitor of the peptide conjugate/MHC complex.

In some embodiments, the polypeptide does not bind to the peptide conjugate/MHC complex with a head-to-head coaxial interaction.

In some embodiments, the polypeptide binds to the peptide conjugate/MHC complex at an angle from about 10° to 60° between the axis of the MHC and the axis of the polypeptide. In some embodiments, when the polypeptide is bound to the peptide conjugate/MHC complex, the angle between the axis of the polypeptide and the axis of the peptide conjugate/MHC complex is from about 10° to 60°; In some embodiments, when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex. In some embodiments, when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex. In some embodiments, the polypeptide binds to the peptide conjugate/MHC complex at an angle that is different than the angle to which a T cell receptor binds to the peptide conjugate/MHC complex. In some embodiments, the polypeptide binds to the peptide conjugate/MHC complex at an angle that is from about 10° to 60° less or from about 10° to 60° more than the angle to which a T cell receptor binds to the peptide conjugate/MHC complex.

In some embodiments, the polypeptide binds to the peptide conjugate/MHC complex at an angle of about 40° between the axis of the MHC and the axis of the polypeptide. In some embodiments, when the polypeptide is bound to the peptide conjugate/MHC complex, the angle between the axis of the polypeptide and the axis of the peptide conjugate/MHC complex is about 40°. In some embodiments, when the polypeptide is bound to the peptide conjugate/MHC complex, the angle between the axis of the polypeptide and the axis of the peptide conjugate/MHC complex is about 40° less or about 40° more than the angle between the axis of a TCR and the axis of the peptide conjugate/MHC complex when the TCR is bound to the peptide conjugate/MHC complex. In some embodiments, the polypeptide binds to the peptide conjugate/MHC complex at an angle that is about 40° less or about 40° more than the angle to which a T cell receptor binds to the peptide conjugate/MHC complex.

In some embodiments, the polypeptide contacts the alpha 1 domain and alpha 2 domain of a heavy chain of the MHC of the peptide conjugate/MHC complex.

In some embodiments, the polypeptide binds to an epitope of the MHC, and wherein the epitope comprises one or more residues from the regions comprising residues 62-66, 106-109, and/or 150-170 of the MHC.

In some embodiments, the polypeptide binds to an epitope of the MHC, and wherein the epitope comprises one or more residues from the regions comprising residues 62-66, 106-109, and/or 150-170 of the HLA-A*03:01 molecule or the HLA-A*11:01 molecule.

In some embodiments, the polypeptide binds to an epitope of the MHC, and wherein the epitope comprises one or more residues selected from the group consisting of residues 62, 106, 108, 109, 158, 161, 162, 162, 165, 166, 167, 169 and 170 of the HLA-A*03:01 molecule.

In some embodiments, the VL domain of the antigen-binding domain binds to an epitope of the MHC, and wherein the epitope comprises one or more residues selected from the group consisting of residues 62, 106, 108, 109, 158, 161, 162, 162, 165, 166, 167, 169 and 170 of the HLA-A*03:01.

In some embodiments, the polypeptide binds to an epitope of the MHC, and wherein the epitope comprises one or more residues selected from the group consisting of residues 62, 65, 66, 150, 151, 154, 155, 157, and 158 of the HLA-A*03:01.

In some embodiments, the VH domain of the antigen-binding domain binds to an epitope of the MHC, and wherein the epitope comprises one or more residues selected from the group consisting of residues 62, 65, 66, 150, 151, 154, 155, 157, and 158 of the HLA-A*03:01.

In some embodiments, the polypeptide binds to an epitope of the MHC, and wherein the epitope comprises one or more residues selected from the group consisting of residues 62, 106, 108, 109, 154, 157, 158, 161, 162, 163, 165, 166, 167, 169 and 170 of the HLA-A*11:01.

In some embodiments, the VL domain of the antigen-binding domain binds to an epitope of the MHC, and wherein the epitope comprises one or more residues selected from the group consisting of residues 62, 106, 108, 109, 154, 157, 158, 161, 162, 163, 165, 166, 167, 169 and 170 of the HLA-A*11:01.

In some embodiments, the polypeptide binds to an epitope of the MHC, and wherein the epitope comprises one or more residues selected from the group consisting of residues 62, 65, 66, 151, 154, 155 and 158 of the HLA-A*11:01.

In some embodiments, the VH domain of the antigen-binding domain binds to an epitope of the MHC, and wherein the epitope comprises one or more residues selected from the group consisting of residues 62, 65, 66, 151, 154, 155 and 158 of the HLA-A*11:01. In some embodiments, the VH is linked to the VL through a linker. In some embodiments, the linker comprises (G₄S)ₙ or (S₄G)ₙ, where n is any integer from 1 to 10. In some embodiments, the linker comprises the glycine-serine-alanine linker G₄SA₃ or a glycine-serine linker (G₄S)₄.

In some embodiments, the polypeptide is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BiTE), bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer. In some embodiments, the polypeptide is a bispecific antibody. In some embodiments, the bispecific antibody is a bispecific T-cell engager (BiTE).

In some embodiments, the polypeptide further comprises a second antigen-binding domain that binds to a T cell surface marker. In some embodiments, the T cell surface marker is CD3 epsilon, CD3 gamma, CD3 delta, CD3 eta, a TCR alpha, or a TCR beta of a TCR. In some embodiments, the antigen-binding domain and the second antigen-binding domain is linked by a linker. In some embodiments, the linker comprises (G₄S)ₙ or (S₄G)ₙ where n is any integer from 1 to 10.

In some embodiments, the linker is configured such that: (a) the polypeptide binds to the peptide conjugate/MHC complex at an angle from about 10° to 60° between the axis of the MHC and the axis of the polypeptide; (b) when the polypeptide is bound to the peptide conjugate/MHC complex, the angle between the axis of the polypeptide and the axis of the peptide conjugate/MHC complex is from about 10° to 60°; (c) when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex; (d) when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex; (e) the polypeptide binds to the peptide conjugate/MHC complex at an angle that is different than the angle to which a T cell receptor binds to the peptide conjugate/MHC complex; and/or (f) the polypeptide binds to the peptide conjugate/MHC complex at an angle that is from about 10° to 60° less or from about 10° to 60° more than the angle to which a T cell receptor binds to the peptide conjugate/MHC complex.

In some embodiments, the polypeptide comprises a first polypeptide chain comprising the antigen-binding domain and a second polypeptide chain comprising the second antigen-binding domain. In some embodiments, the first or the second polypeptide chain is further fused to a cytokine or fragment thereof. In some embodiments, the cytokine comprises IL-2, IL-7, IL-15, IL-12, IL-18, or IL-21, or an interferon (IFN). In some embodiments, the peptide conjugate/MHC complex is presented on a surface of a cell. In some embodiments, the cell expresses a low copy number of the peptide conjugate/MHC complex, and wherein the low copy number is at most about 1000, 900, 800, 700, 600, 500, 400, 300, 200, 100, 90, 80, 70, 60, 50, 40, 30, 20, 10, 5, 3, or 1 copy per a single cell.

In some embodiments, the peptide of the peptide conjugate/MHC complex is from an intracellular protein.

In some embodiments, the polypeptide is the binding partner disclosed herein.

In another aspect, the present disclosure provides a polypeptide comprising an antigen binding domain that specifically binds to a peptide conjugate/MHC complex, wherein the peptide conjugate/MHC complex comprises: (a) a peptide conjugate comprising the peptide covalently linked to a targeted covalent inhibitor or fragment thereof; and (b) an MHC.

In another aspect, the present disclosure provides a pharmaceutical composition comprising a polypeptide disclosed herein and a pharmaceutically acceptable carrier.

In another aspect, the present disclosure provides a method of treating cancer in a subject that has been treated with a free targeted covalent inhibitor, the method comprising administering to the subject the polypeptide disclosed herein or the pharmaceutical composition disclosed herein. In some embodiments, the subject is refractory to a treatment with the free targeted covalent inhibitor.

In another aspect, the present disclosure provides a method of treating cancer in a subject, the method comprising administering to the subject the polypeptide disclosed herein or the pharmaceutical composition disclosed herein after or simultaneously with administration of a small molecule drug.

In another aspect, the present disclosure provides a method of treating cancer in a subject, the method comprising administering to the subject the targeted covalent inhibitor, and administering to the subject the polypeptide disclosed herein or the pharmaceutical composition disclosed herein. In some embodiments, the polypeptide disclosed herein or the pharmaceutical composition disclosed herein is administered after administration of the targeted covalent inhibitor or simultaneously with the targeted covalent inhibitor.

In another aspect, the present disclosure provides a computer-assisted method for identifying or designing a potential polypeptide comprising an antigen binding domain that binds to a peptide conjugate/MHC complex, wherein the peptide conjugate of the peptide conjugate/MHC complex is a peptide covalently linked to a targeted covalent inhibitor or fragment thereof, the method comprising: (a) providing the coordinates of at least two atoms of (i) the peptide conjugate/MHC complex of the FIGS. 57A-C, 58A-B, 59A-C, 60A-B and 61, (b) providing the structure of a candidate polypeptide comprising an antigen binding domain for binding to the peptide conjugate/MHC complex, (c) fitting the structure of the candidate polypeptide to the at least two atoms of the peptide conjugate/MHC complex, wherein fitting comprises determining interactions between one or more atoms of the antigen binding domain of the candidate polypeptide and atoms of the peptide conjugate/MHC complex, and (d) selecting the candidate polypeptide if it is predicted to bind to the peptide conjugate/MHC complex.

In another aspect, the present disclosure provides a computer-assisted method for designing a potential polypeptide comprising an antigen binding domain that binds to a peptide conjugate/MHC complex, wherein the peptide conjugate of the peptide conjugate/MHC complex is a peptide covalently linked to a targeted covalent inhibitor or fragment thereof, the method comprising: (a) providing the structure of a candidate polypeptide comprising an antigen binding domain for binding to a peptide conjugate/MHC complex, (b) providing the coordinates of at least two atoms of the peptide conjugate/MHC complex and the coordinates of at least two atoms of an antigen binding domain of a polypeptide that is bound to the peptide conjugate/MHC complex of the FIGS. 57A-C, 58A-B, 59A-C, 60A-B and 61, (c) fitting the structure of the candidate polypeptide to the at least two atoms of the peptide conjugate/MHC complex and the at least two atoms of an antigen binding domain of a polypeptide that is bound to the peptide conjugate/MHC complex, wherein fitting comprises determining interactions between one or more atoms of the antigen binding domain of the candidate polypeptide and atoms of the peptide conjugate/MHC complex, and (d) selecting the candidate polypeptide if it is predicted to bind to the peptide conjugate/MHC complex.

In another aspect, the present disclosure provides a computer-assisted method for designing a potential polypeptide comprising an antigen binding domain that binds to a peptide conjugate/MHC complex, wherein the peptide conjugate of the peptide conjugate/MHC complex is a peptide covalently linked to a targeted covalent inhibitor or fragment thereof, the method comprising: (a) providing the structure of a polypeptide comprising an antigen binding domain bound to a peptide conjugate/MHC complex, (b) determining interactions between one or more atoms of the antigen binding domain of the polypeptide and one or more atoms of the peptide conjugate/MHC complex, (c) providing a candidate polypeptide comprising an antigen binding domain for binding to a peptide conjugate/MHC complex, wherein the antigen binding domain of the candidate polypeptide comprises one or more amino acid substitutions relative to the polypeptide comprising an antigen binding domain, wherein the one or more amino acid substitutions are of residues of the antigen binding domain of the polypeptide that comprise the one or more atoms that interact with or modulate interaction with the one or more atoms of the peptide conjugate/MHC complex, and (d) selecting the candidate polypeptide if it binds or is predicted to bind to the peptide conjugate/MHC complex with a higher affinity than the affinity of the polypeptide comprising an antigen binding domain to the peptide conjugate/MHC complex.

In another aspect, the present disclosure provides a computer-assisted method for identifying or designing a potential polypeptide comprising an antigen binding domain that binds to a peptide conjugate/MHC complex, wherein the peptide conjugate of the peptide conjugate/MHC complex is a peptide covalently linked to a targeted covalent inhibitor or fragment thereof, the method comprising: using a computer system, e.g., a programmed computer comprising a processor, a data storage system, an input device, and an output device, the steps of: (a) inputting into the programmed computer through said input device data comprising the three-dimensional coordinates of a subset of the atoms from or pertaining to a crystal structure of FIGS. 57A-C, 58A-B, 59A-C, 60A-B and 61, thereby generating a data set, (b) comparing, using said processor, said data set to a computer database of structures stored in said computer data storage system structures of polypeptides comprising an antigen binding domain that bind or putatively bind or that are desired to bind to a peptide conjugate/MHC complexes, (c) selecting from said database, using computer methods, structure(s) that may bind to certain peptide conjugate/MHC complexes, (d) constructing, using computer methods, a model of the selected structure(s), and (e) outputting to said output device the selected structure(s); and optionally synthesizing one or more of the selected structure(s); and further optionally testing said synthesized selected structure(s) for binding a peptide conjugate/MHC complex.

In another aspect, the present disclosure provides a computer-readable media containing: atomic coordinate data according to the structure of any one of FIGS. 57A-C, 58A-B, 59A-C, 60A-B and 61, said data defining the three dimensional structure or providing the structure of a polypeptide comprising an antigen binding domain bound to a peptide conjugate/MHC complex, wherein the peptide conjugate of the peptide conjugate/MHC complex is a peptide covalently linked to a targeted covalent inhibitor or fragment thereof, or at least one sub-domain thereof, or structure factor data for the polypeptide comprising an antigen binding domain bound to the peptide conjugate/MHC complex, said structure factor data being derivable from the atomic coordinate data of any one of FIGS. 57A-C, 58A-B, 59A-C, 60A-B and 61.

In another aspect, the present disclosure provides a computer-readable media containing: atomic coordinate data according to the structure of any one of FIGS. 57A-C, 58A-B, 59A-C, 60A-B and 61, said data defining the three dimensional structure or providing the structure of a peptide conjugate/MHC complex, wherein the peptide conjugate of the peptide conjugate/MHC complex is a peptide covalently linked to a targeted covalent inhibitor or fragment thereof, or at least one sub-domain thereof, or structure factor data for the peptide conjugate/MHC complex, said structure factor data being derivable from the atomic coordinate data of the structure of any one of FIGS. 57A-C, 58A-B, 59A-C, 60A-B and 61.

In another aspect, the present disclosure provides a method of identifying a T-cell receptor (TCR) that recognizes the peptide conjugate/MHC complex disclosed herein, the method comprising: (a) contacting a plurality of candidate TCRs with the peptide conjugate/MHC complex, and (b) identifying at least one TCR that binds to the peptide conjugate/MHC complex.

In another aspect, the present disclosure provides a method of identifying a T-cell receptor (TCR) that recognizes the peptide conjugate/MHC complex disclosed herein, the method comprising: (a) culturing T cells and antigen presenting cells (APCs) treated with the targeted covalent inhibitor, thereby generating peptide conjugate/MHC complex specific T cells with a T cell receptor (TCR) that binds to the peptide conjugate/MHC complex; and (b) identifying at least one TCR from the peptide conjugate/MHC complex specific T cells that binds to the peptide conjugate/MHC complex. In some embodiments, the method disclosed herein further comprises selecting or isolating the at least one TCR.

In some embodiments, the plurality of candidate TCRs is a plurality of soluble TCRs or a plurality of TCRs expressed on cell surface of a plurality of cells. In some embodiments, the plurality of candidate TCRs is the plurality of TCRs expressed on cell surface of the plurality of cells, and identifying in (b) comprises isolating or selecting a cell comprising the at least one TCR based on an activation marker of the cell. In some embodiments, the activation marker is a T cell activation marker. In some embodiments, the T cell activation marker is CD26, CD27, CD28, CD30, CD154, CD40L, CD134. CD25, CD44, CD69, CD137, or KLRG1.

In another aspect, the present disclosure provides a T-cell receptor (TCR) comprising the at least one TCR disclosed herein. In some embodiments, the TCR is a soluble TCR. In some embodiments, the TCR is a bispecific TCR.

In some aspects disclosed herein is a method of treating cancer. In some embodiments, the method comprises prophylactically administering a peptide conjugate to a subject in need thereof prior to the subject developing a cancer, or prior to the subject being administered a drug, or both, wherein the peptide conjugate comprises the peptide covalently linked to a targeted covalent inhibitor or fragment thereof.

### BRIEF DESCRIPTION OF THE FIGURES

FIGS. 1A-C. Graphs depicting results of phage enzyme-linked immunosorbent assay (ELISA) of phage-displayed antibody clones. Binding to KRAS^{G12C}-GDP and KRAS^{G12C}-GDP-ARS-1620 conjugate was determined. For each clone, the bars in the graph are, from left to right, Buffer, G12C-GDP-ARS, and G12C-GDP. Results for clones 1-32 are represented in FIG. 1A, results for clones 33-64 in FIG. 1B, and results for clones 65-96 in FIG. 1C.

FIG. 2. Graph showing 12C-ARS Fab59 binding to KRAS^{G12C} in GTPγS- or GDP-bound nucleotide state with or without ARS-1620 was characterized using the bead binding assay.

FIG. 3. Graphs showing that 12C-ARS-Fab59 specifically binds KRAS^{G12C}-GDP conjugated to ARS-1620.

FIGS. 4A-D. Shown in FIG. 4A is data obtained using 12C-ARS-Fab59 to measure ARS-1620/KRAS^{G12C} adducts by pull-down assays from lysates prepared from cell lines. Shown in FIG. 4B are immunoblots of whole cell lysates and 12C-ARS Fab-pull-downs (PD) from RAS-less MEFs reconstituted with the indicated KRAS mutants (FIG. 4A) and KCP (Kras^{G12C}; Tp53^{R172H}; Pdx-Cre) mouse pancreas cancer cells (FIG. 4B), treated in the presence or absence of ARS-1620. Shown in FIG. 4C are whole cell lysates and 12C-ARS Fab pull-downs (PD) from H358 and MIAPaCa-2 cells, treated as indicated. Shown in FIG. 4D is ARS-adduct formation in samples from FIG. 4C, quantified by LC/MS-MS assay. ARS-1620 and SHP099 concentrations were 10 μM in all panels.

FIGS. 5A and 5B. Data showing that 12C-ARS-Fab59 can be used to measure the engagement of ARS-1620 to mutant KRAS by pull-down assay with lysates prepared from animal tissues. FIGS. 5A and 5B show anti-pan RAS and anti-ERK2 (loading control) immunoblots of lysates and 12C-ARS Fab pull-downs (PD) from LSL-KRAS^{G12C}-Tp53^{R270H} (FIG. 5A) and LSL-KRAS^{G12C} (FIG. 5B) tumors after 3 days of oral gavage with ARS-1620 (200 mg/kg/d) alone or with the SHP2 inhibitor SHP099 (75 mg/kg/d).

FIG. 6. Binding of antibody clones to AMG-510 conjugated to the KRAS(G12C) peptide and the poly-Ser (unrelated) peptide. For each antibody clone, the bars are from left to right are No target, KRAS(G12C), KRAS(G12C)-AMG, and Unrelated peptide-AMG. Signals for the two negative controls, no target and the KRAS(G12C) peptide without a conjugated drug, were too low to be visible in the graph. The antibody clones were displayed on the yeast cell surface, and binding of the targets conjugated to fluorescently labeled streptavidin was detected by using flow cytometry.

FIG. 7. Binding of the P2AMR-1 clone in the human IgG1 format to AMG-510 conjugated to the KRAS(G12C) peptide (circles) and the same peptide without drug conjugation (squares). The peptide was immobilized on streptavidin-coated beads, and antibody bound to the beads was detected with a fluorescently labeled secondary antibody. The apparent K_{D} value is shown. Data shown here are from triplicate measurements. Error bars are within the size of the symbols.

FIG. 8. Recognition of AMG-510 presented on class I MHC molecule. The AMG510-RAS(G12C) conjugate (circles) and unconjugated peptide (squares) were loaded onto HLA-A*03:01 and immobilized on streptavidin-coated beads. Antibody binding was detected using the same method as in FIG. 2. The apparent K_{D} value is shown. Data shown here are from triplicate measurements. Error bars are within the size of the symbols.

FIG. 9. Cartoon representation of the disclosed concept referred to as HapImmune™.

FIGS. 10A-C. Data representing development of antibodies that bind MHC/peptide-drug conjugate complexes. FIG. 10A shows multiplex bead-binding assay (MBBA) of phages displaying different antibody clones. FIG. 10B shows MBBA assay of phages displaying different antibody clones to: HLA-A*01:01 in complex with the BTK peptide conjugated to Ibrutinib. FIG. 10C shows MBBA assay of phages displaying different antibody clones to: HLA-A*02:01 in complex with the EGFR peptide conjugated to osimertinib.

FIGS. 11A-C. Binding titration graphs using the multiplex bead-binding assay (MBBA) of purified antibodies targeted to the KRAS(G12C)-AMG510 conjugate. Clone names are shown over each graph. Graphs for clones AMRA3-7 hIgG1, AMRA3-18 hIgG1, and AMRA3-22 hIgG1 appear in FIG. 11A; graphs for clones AMRA11-2 hIgG1 and AMRA11-15 hIgG1 appear in FIG. 11B; graphs for clones AMRA311-16 hIgG1, AMRA311-17 hIgG1, and AMRA311-18 hIgG1 appear in FIG. 11C. Antigen nomenclature is described in FIG. 10.

FIG. 12. Graph showing that binding of antibodies to the AMG510-peptide conjugate in complex with an HLA was not affected by the presence of the free drug, AMG-510.

FIG. 13. Graphs of binding titrations using the multiplex bead-binding assay (MBBA) of purified antibodies targeted to the BTK-Ibrutinib conjugate. Clone names are shown over each graph. Antigen nomenclature is described in FIG. 10.

FIGS. 14A and 14B. Graphs of binding titrations of purified antibodies to the KRAS(G12C)-AMG510 conjugate presented by endogenous HLA molecules on the cell surface. Raji cells were first incubated with the KRAS(G12C)-AMG510 conjugate or the KRAS (wild type) peptide, and excess conjugate and peptide were washed away. The antibody levels detected using a fluorescently labeled secondary antibody are shown as a function of IgG concentration used for staining. Apparent dissociation constant (K_{D}) values were determined using nonlinear least-squared fitting of a 1:1 binding function. Data shown here are from triplicate measurements. Graphs for antibodies AMRA3-7 hIgG1, AMRA3-22 hIgG1, AMRA3-18 hIgG1, and AMRA11-2 hIgG1 appear in FIG. 14A; graphs for antibodies AMRA3-7 hIgG1, AMRA3-22 hIgG1, AMRA3-18 hIgG1, and AMRA11-2 hIgG1 appear in FIG. 14B.

FIGS. 15A-C. Antibody binding to a KRAS(G12C)-expressing cell line pretreated with AMG-510. FIG. 15A shows flow cytometry histograms. FIG. 15B shows quantification of the median fluorescence intensity of H358 cells treated with or without AMG-510. FIG. 15C shows quantification of the median fluorescence intensity of H358 cells and HEK293T cells (a negative control) treated with or without AMG-510 and stained with the AMRA3-7 antibody.

FIG. 16. Graph showing binding of P2AMR-1 IgG to cells preincubated with the KRAS(G12C) peptide-AMG510 conjugate, KRAS (wild type) peptide, or no peptide.

FIGS. 17A and 17B. Graphs showing binding of purified antibodies in the IgG format to the indicated drug-peptide/HLA complexes as measured using the multiplex bead binding assay (MBBA). FIG. 17A shows antibody clones identified with AMG-510 conjugated to KRAS(G12C) peptide in complex with HLA-A*03:01 as the antigen. FIG. 17B shows antibody clones identified with AMG-510 conjugated to KRAS(G12C) peptide in complex with HLA-*11:01 as the antigen.

FIGS. 18A and 18B. Graphs showing the cytotoxic effect of single-chain Diabodies (scDbs) on cells pulsed with an exogenous peptide-drug conjugate. FIG. 18A shows Raji cells that were first pulsed with AMG-510 conjugated to a peptide corresponding to a fragment of KRAS(G12C) or a control peptide corresponding to KRAS (wild type). The pulsed cells were co-cultured with human T cells (Effector:Target=3:1) in the presence of single-chain Diabodies (scDbs) at the indicated concentrations. After incubation, dead cells were stained and detected by flow cytometry. Data shown here are from triplicate measurements. Error bars indicate the s.d. Where error bars are not visible, the errors are smaller than the symbols. FIG. 18B shows equivalent experiments using T2 cells and osimertinib conjugated to an EGFR peptide. As a negative control, peptide conjugated with beta-mercaptoethanol was used.

FIGS. 19A and 19B. Graphs showing specific cytotoxic effect of AMRA3-7_UCHT1 scDb on drug-treated lung cancer cell lines. FIG. 19A shows lung cancer cell lines that were treated with 100 nM AMG-510 for 24 hr, then co-cultured with human T-cells (E:T=5:1) in the presence of AMRA3-7_UCHT1 scDb. After incubation, cell viability was measured. The scDb antibody showed dose-dependent cytotoxic effects only on AMG510-treated cells with the cognate KRAS mutation (G12C) and HLA (HLA-A3). FIG. 19B shows cytotoxic effect of scDb at 0.1 nM concentration. Data shown here are from quadruplicate measurements.

FIG. 20. Binding titration curves are shown of AMR-A3-7 and AMR-A3-7D displayed on the yeast cell surface. Binding to HLA-A*03:01 presenting the 9-mer or 10-mer RAS(G12C) peptides, VVGACGVGK and VVVGACGVGK respectively, with AMG-510 conjugated to their Cys residues.

FIGS. 21A and 21B. Graphs showing cell killing effects of AMRA3-7D scDb. FIG. 21A shows dose-dependent cell killing effect of AMRA3-7D scDb tested with Raji cells that were first pulsed with AMG-510 conjugated to a peptide corresponding to a fragment of KRAS(G12C) (RAS-AMG510) or a control peptide corresponding to KRAS (wild type, WTRAS). FIG. 21B shows cell killing effect of AMRA3-7D scDb tested against H2122 non-small cell lung cancer cell line treated with AMG-510 or DMSO only (negative control).

FIGS. 22A-C. Cell binding and killing effects of AMRA3-7D CrossMab. FIG. 22A shows binding of AMRA3-7D to Jurkat cells, which express CD3, and to Raji cells, which do not express CD3. FIG. 22B shows dose-dependent cell killing effect of AMRA3-7D CrossMab tested with Raji cells that were first pulsed with AMG-510 conjugated to a peptide corresponding to a fragment of KRAS(G12C) (RAS-AMG510) or a control peptide, corresponding to KRAS (wild type, WTRAS). FIG. 22C shows cell killing effect of AMRA3-7D CrossMab tested with H2122 non-small cell lung cancer cell line treated with AMG-510 or DMSO only (negative control).

FIGS. 23A and 23B. Deep mutational scanning of AMR-A3-7D. FIG. 23A shows representative flow cytometry profiles of yeast cells displaying AMRA3-7D and its deep mutational scanning library derivatives. FIG. 23B shows the prevalence of mutations at each position in the sorted subsets of the deep mutational scanning library is shown in a heat map format.

FIGS. 24A and 24B. Deep mutational scanning of OEA2-5. FIG. 24A shows representative flow cytometry profiles of yeast cells displaying OEA2-5 in the single-chain Fv format and its deep mutational scanning library derivatives. FIG. 24B shows the prevalence of mutations at each position in the sorted subsets of the deep mutational scanning library in a heat map format.

FIG. 25. Binding of antibody clones in the single-chain Fv format to an AMG510-KRAS(G12C) peptide conjugate in complex with HLA-A*11 measured in yeast display format. The binding intensity is shown in terms of median fluorescence intensity (MFI) in arbitrary units.

FIGS. 26A-F. Binding titration of select clones to the AMG510-KRAS(G12C) peptide conjugates in complex with HLA-A*03 (top row; FIGS. 26A-C) and with HLA-A*11 (bottom row; FIGS. 26D-F), tested in a yeast display format. Data with the 9mer peptide, VVGAC*GVGK, where C* denotes Cys residue conjugated with AMG-510, are shown with the closed circles, and data with the 10mer peptide, VVVGAC*GVGK, are shown with the open circles. Data with the wild-type 9mer, VVGAGGVGK, are shown with the closed triangles. The curves are the best fits of the 1:1 binding model.

FIGS. 27A-D. FIGS. 27A-C show comparisons of binding of antibody clones to the AMG510-KRAS(G12C) peptide (9mer) presented on HLA-A*03 (closed circles) with that to AMG-510-KRAS(G12C) peptide in isolation, i.e. in the absence of HLA-A*03 (open boxes). FIG. 27D shows binding of antibodies to the AMG510-KRAS(G12C) peptide conjugate presented on HLAs in the presence and absence of 10 μM free AMG-510, demonstrating that these antibodies are not inhibited by the presence of excess concentrations of free AMG-510.

FIG. 28. Cytotoxic effect of scDb antibodies on AMG510-treated lung cancer cells. NCI-H2122 cells expressing luciferase were incubated with 1 μM AMG-510 or DMSO (vehicle), and then co-cultured with human T-cells (E:T=10:1) in the presence of scDbs. After incubation, the death of target cells was assessed by measuring the activity of luciferase released into the media from dead cells. The affinity-matured antibodies showed potent cytotoxic effects in a manner dependent on AMG-510 treatment. The concentration of scDbs was 10 nM except that 5 nM was used for anti-HLA-A3 scDb (a positive control scDb targeted to HLA-A*03). Data shown here are from quadruplicate measurements.

FIG. 29. Cytotoxic effect of scDb antibodies on AMG510-treated lung cancer cells. Top, Dose titration of three different scDb antibodies. Bottom, cytotoxic effects at 10 nM scDb compared with 5 nM control scDb. Experiments were performed in the same manner as for FIG. 28.

FIG. 30 shows Table E representing combinations of mutations that exhibited improved binding.

FIG. 31 shows Table F representing combinations of mutations that exhibited improved binding.

FIG. 32. Binding titration of clones exhibiting reduced affinity to the AMG510-KRAS(G12C) peptide conjugates in complex with HLA-A*03, relative to affinity of the parental AMR3-7-D clone, tested in a yeast display format. Experiments were performed in the same manner as for FIG. 26.

FIG. 33 shows a table representing combinations of mutations that exhibited reduced binding as shown in FIG. 32.

FIG. 34 shows a table representing combinations of mutations that exhibited reduced binding as shown in FIG. 32.

FIG. 35. Biolayer interferometry (BLI) measurements of RA_D11 to the AMG510-KRAS(G12C) peptide conjugates in complex with HLA-A*03 (left panels) and with HLA-A*11 (right panels). Sensorgrams with the HLA complexes at the indicated concentrations with the 9mer peptide, VVGAC*GVGK, where C* denotes Cys residue conjugated with AMG-510, are shown in black in the upper panels. Data with the 10mer peptide, VVVGAC*GVGK, are shown in the lower panels. Data with the HLA complexes at 64 nM with the wild-type 9mer, VVGAGGVGK, and 10mer, VVVGAGGVGK, are shown in gray. RA_D11 in the biotinylated Fab format was immobilized on the BLI tip and reacted with HLA complexes in solution. The K_{D} values were estimated by global curve fitting of a 1:1 binding model.

FIG. 36. BLI sensorgrams showing binding of RA_D11 Fab to an AMG510-KRAS(G12C) peptide conjugate in complex with HLA-A*02. Sensorgrams with the HLA complex at the indicated concentrations with the 10mer peptide, KLVVVGAC*GV, where C* denotes Cys residue conjugated with AMG-510. The K_{D} value was estimated by global curve fitting of a 1:1 binding model.

FIG. 37. Specific cytotoxic effect of the RA_D11 scDb antibody on H2122 (left panel) and SW1573 (right panel) lung cancer cell lines, which express KRAS(G12C), pretreated with 1 μM AMG-510. Anti-pan HLA-A*03 scDb (A3-2) was used as a positive control. Note that it also binds to HLA-A02, albeit less tightly than to HLA-A*03. The experiments were performed in the same manner as for FIG. 28.

FIG. 38. Lack of cytotoxic effect of the AMRA3-7 and RA_D11 scDb antibodies on AMG510-treated Raji cells, which express wild-type KRAS, as expected. Raji cells were pretreated with 1 μM AMG-510 and stained with carboxyfluorescein succinimidyl ester (CSFE). The cells were then co-cultured with human T-cells (E:T=5:1) in the presence of scDbs for 18 hr, harvested, stained with Fixable Viability Dye eFluor660, and analyzed by flow cytometry.

FIG. 39. Binding titration of EO_Q16 and EO_Q17 to osimertinib-EGFR/HLA-A*02 (filled circles) and to EGFR/HLA-A*02 (no drug-conjugation; open circles), showing that these antibodies have high affinity to their intended antigen but not to the equivalent antigen without drug conjugation.

FIG. 40. Binding of EO_Q16 and EO_Q17 to osimertinib-EGFR/HLA-A*02 (10 nM) in the absence and presence of free osimertinib (1 μM), showing that antigen binding of these antibodies is not inhibited by the presence of free drug.

FIG. 41. Cytotoxic effect of scDb antibodies on the cells spiked with the drug-peptide conjugates. EO_Q16 and EO_Q17 scDbs showed potent cytotoxic effects on the cells spiked with the osimertinib-EGFRb peptide conjugate but not on the cells treated with the same peptide without drug conjugation. BB7.2 scDb is an anti-pan HLA-A2 clone, which was used as a positive control. Data shown are from triplicate measurements.

FIG. 42. Cytotoxic effects of scDb antibodies on OCI-AML3 cells treated with osimertinib. EO_Q16 and EO_Q17 scDb antibodies did not show cytotoxic effects, as expected, because OCI-AML3 cells do not possess an osimertinib target. BB7.2 scDb is an anti-pan HLA-A2 clone, which was used as a positive control. Data shown are from triplicate measurements.

FIGS. 43A-H. Cytotoxic effects of RA_D11 scDb on sotorasib-treated tumor cells. FIG. 43A shows dose response curves of the viability of H358 and H2122 cells following exposure to sotorasib for 72 hr. FIG. 43B shows analysis of sotorasib conjugation to KRAS(G12C) in H2122 cells by Western blot. H2122 cells were incubated with 100 nM sotorasib for 24 hr. The arrow indicates KRAS(G12C) conjugated to sotorasib. Note that the anti-pan-RAS antibody detects KRAS, HRAS, and NRAS, so complete shift of the original band is not expected. FIG. 43C shows cytotoxic effects of the indicated scDbs on H2122-Nluc cells treated with 1 μM sotorasib. The fold-changes in luminescence signal intensity, normalized to that from the no scDb control (i.e., H2122 and T cells only), are shown. The scDb concentration was 10 nM except for the A3-2 scDb (1 nM). DMSO is the vehicle for sotorasib. FIG. 43D shows cell killing titration curve of the RA_D11 scDb on H2122-Nluc cells treated with 1 μM sotorasib. FIG. 43E shows dependence of cell killing on sotorasib concentration with the indicated scDbs at 1 nM. FIG. 43F shows HLA dependence of cell killing by RA_D11 scDb. The normalized luminescence intensity is shown for cell lines treated with 0.3 μM sotorasib and cocultured with T cells in the presence of 1 nM scDbs and 0.3 μM sotorasib. KRAS mutation state and HLA alleles for the cell lines are shown. Because each cell line expresses a different level of Nluc, assays were performed separately, and relative luciferase levels were determined for each cell line. FIGS. 43G and 43H show cytotoxic effects of the RA_D11 scDb (1 nM) on H2030-Nluc (G) and SW1573-Nluc (H) cells treated with sotorasib. Data shown are from quadruplicate measurements.

FIGS. 44A and 44B. HLA expression of cell lines. FIG. 44A shows analysis of HLA-A*03 expression on Raji cells. FIG. 44B shows analysis of HLA-A*03 expression on H2122 cells and H2122 (HLA-A*03KO) cells. Note the lack of HLA-A*03 in the latter, indicating successful deletion.

FIGS. 45A-D. Cytotoxic effects of RA_D11 on sotorasib-treated tumor cells. FIG. 45A shows cytotoxic effects of the indicated scDbs on H2122-Nluc cells treated with 1 mM sotorasib. The scDb concentration was 10 nM except for A3-2 scDb, a positive control (1 nM). FIG. 45B shows cytotoxic effects of the indicated scDbs on various tumor cell lines expressing NLuc treated with 0.3 μM sotorasib. The scDb concentration was 1 nM. FIGS. 45C and 45D show cytotoxic effect of the RA_D11 scDb on H2030-Nluc cells (FIG. 45C) and SW1573-Nluc (FIG. 45D) treated with sotorasib. The scDb concentration was 1 nM. Anti-pan HLA-A*03/A*02 antibody (A3-2 scDb), anti-pan HLA-A*02 antibody (BB7.2 scDb), and anti-pan HLA-A*11 antibody (A11-1) were used as positive controls. Data shown are from quadruplicate measurements.

FIG. 46. Binding analysis of antibody phage clones to peptide/MHC complexes or sotorasib/peptide/MHC complexes by the multiplex bead binding assay (MBBA). Phage MBBA signals show the binding specificity of antibody clones (A3-2 and A11-1) that are not specific to the identity of the bound peptide.

FIGS. 47A-C. Structures of soto-pMHC-RA_D11 complexes (side and top views). FIG. 47A shows a side view structure (top panel) and a top view structure (bottom panel) of soto-p₈/A03-RA_D11. FIG. 47B shows a side view (top panel) structure and a top view (bottom panel) structure of soto-p₇/A03-RA_D11. FIG. 47C shows a side view (top panel) structure and a top view (bottom panel) structure of soto-p₇/A11-RA_D11. Proteins and peptides are represented with tube diagrams, sotorasib with sticks. 4701 indicates the relative region of RA_D11 VH, 4702 indicates the relative region of RA_D11 VL, 4703 indicates the relative region of β2m, 4704 indicates the relative region of HLAs, and 4705 indicates the relative position of the drug-peptide conjugate soto-p. The angle between the axes of Fab RA_D11 and soto-pMHCs is shown in the soto-p₈/A03 structure (FIG. 47A).

FIG. 48. Surface areas of residues buried following interaction of Fab RA_D11 and soto-pMHCs, as calculated by PDBePISA. The shades of grey indicate the different percentages of solvent-accessible residue surfaces buried upon interaction of the indicated molecules, as shown on the scale bar on the right (darker grey, higher percentage of Buried Surface Area, BSA).

FIGS. 49A-E. Details of interaction between Fab RA_D11 and soto-p₈/A03. FIG. 49A shows a tube representation of HLA-A*03:01 (tubes) presenting soto-p₈ (sticks; e.g., V₈V₉G₁₀A₁₁C₁₂G₁₃V₁₄G₁₅K₁₆-sotorasib) in complex with Fab RA_D11 (not shown). HLA-A*03:01 residues contacting either Fab RA_D11 or sotorasib are represented as sticks. FIG. 49B shows contact areas between the HLA molecule and Fab RA_D11. Contact areas of Fab RA_D11 (4901 shows the approximate area for contacting VL, and 4902 shows the approximate area for contacting VH), sotorasib (4903 shows the approximate area for contacting sorotasib) or both (4904) depicted on the surface of HLA-A*03:01 are labeled. FIG. 49C shows a tube representation of Fab RA_D11 in complex with soto-p₈/A03 (only sotorasib shown; sticks). Fab RA_D11 residues contacting either HLA-A*03:01 or sotorasib are represented as sticks. FIG. 49D shows contact areas of HLA-A*03:01 (4905), sotorasib (4906), or both (4907, the area surrounding sotorasib) depicted on the surface of Fab RA_D11. FIG. 49E shows detail of HLA-A*03:01 residues (e.g., R₁₀₈, D₁₆₁, E₁₆₆, W₁₆₇, R₁₆₈) forming H-bonds with Fab RA_D11 (e.g., Q₂₇, S₂₉, S₃₀, S₃₁, Y₉₃). Residues involved in the H-bonds are shown as sticks, H-bonds as dashed lines.

FIGS. 50A-E. Interaction details of Fab RA_D11 binding to soto-p₇/A03. FIG. 50A shows a tube representation of HLA-A*03:01 (tubes) presenting soto-p₇ (sticks; e.g., V₇V₈V₉G₁₀A₁₁C₁₂G₁₃V₁₄G₁₅K₁₆-sotorasib) in complex with Fab RA_D11 (not shown). HLA-A*03:01 residues contacting either Fab RA_D11 or sotorasib are represented as sticks. FIG. 50B shows contact areas of Fab RA_D11 (5001 shows the approximate area for contacting VH; 5002: VL; 5003: VH and VL), sotorasib (5004) or both (5005) depicted on the surface of HLA-A*03:01. FIG. 50C shows a tube representation of Fab RA_D11 in complex with soto-p7/A03 (only sotorasib shown; stick). Fab RA_D11 residues contacting either HLA-A*03:01 or sotorasib are represented as sticks. FIG. 50D shows contact areas of HLA-A*03:01 (5006), sotorasib (5007) or both (5008) depicted on the surface of Fab RA_D11. FIG. 50E shows detail of HLA-A*03:01 (e.g., E₁₆₆, W₁₆₇, R₁₀₈) residues forming h-bonds with Fab RA_D11 VH (e.g., S33) and with RA_D11 VL (e.g., Y₉₃, S₉₂, S₂₈, S₃₀). Residues involved in the h-bonds are shown as sticks, H-bonds as black dashed lines.

FIGS. 51A-F. Interaction details of Fab RA_D11 binding to soto-p₇/A11. FIG. 51A shows tube representation of HLA-A*11:01 (tubes) presenting soto-p₇ (sticks; e.g., V₈V₉G₁₀A₁₁C₁₂G₁₃V₁₄G₁₅K₁₆-sotorasib) in complex with Fab RA_D11 (not shown). HLA-A*11:01 residues contacting either Fab RA_D11 or sotorasib are represented as sticks. FIG. 51B shows contact areas of Fab RA_D11 (5101 shows the approximate area for contacting VH; 5102: VL; 5103: VH and VL), sotorasib (5104) or both (5105) depicted on the surface of HLA-A*11:01. FIG. 51C shows tube representation of Fab RA_D11 in complex with soto-p7/A11 (only sotorasib shown; sticks). Fab RA_D11 residues contacting either HLA-A*11:01 or sotorasib are represented as sticks. FIG. 51D shows contact areas of HLA-A*11:01 (5106), sotorasib (5107) or both (5108) depicted on the surface of Fab RA_D11. FIGS. 51E and 51F show details of sotorasib and HLA-A*11:01 (e.g., E₁₆₆, R₁₆₉, R₁₀₈, D₁₀₆) residues forming H-bonds with Fab RA_D11 VH (e.g., G100, A103) and RA_D11 VL (e.g., K₉₆, S₉₂, R₉₅, Q₂₇, S₂₈, G₆₈, R₆₆). Residues involved in the H-bonds are shown as sticks, H-bonds as black dashed lines.

FIGS. 52A-F. Comparison of the different Fab RA_D11-soto-pMHCs structures. FIGS. 52A and 52B show superimposition of structures of soto-p₈/A03 and soto-p₇/A03. HLA-A*03:01 is represented in tubes. Side chains of HLA residues involved in RA_D11 interactions are represented in sticks. FIG. 52B shows detail of the comparison between soto-p₈ (dark) and soto-p₇ (light) when loaded by HLA-A*03:01 (not shown) and bound to Fab RA_D11 (not shown). FIGS. 52C-F show superimposition of structures of soto-p₇/A03 and soto-p₇/A11. Soto-pz is represented in sticks (light), and HLA-A*03:01 (light) HLA-A*11:01 (dark) are represented in tubes. Side chains of HLA residues different between HLA-A*03 and A*11 are represented in sticks. FIG. 52D shows detail of the comparison between soto-p₇ peptides presented by HLA-A*03:01 (light sticks, HLA not shown) or HLA-A*11:01 (dark sticks, HLA not shown) and bound to Fab RA_D11 (not shown). FIG. 52E shows detail of soto-p₇ presentation differences caused by T163/R163 presence in HLA-A*03/A*11. FIG. 52F shows detail of Fab RA_D11 CDRH3 different conformations when binding soto-p₇/A03 or soto-p₇/A11.

FIG. 53. Flow cytometry profiles of RA_D11 (left column) binding to drug-peptide/MHC complexes and to sorted deep mutational scanning libraries. Condition 1:10 nM soto-p₇/A03; condition 2:10 nM soto-p₇/A11; condition 3:10 nM soto-p₅/A02 tetramer (on streptavidin-DyLight650).

FIG. 54. Heatmap representation of single-point mutants in VL of RA_D11 that retained antigen binding (left column) or that lost binding (right column). The numbers indicate number of reads for a mutation of interest divided by the total number of reads for that position multiplied by 100. Condition 1:10 nM soto-p₇/A03; condition 2:10 nM soto-p7/A11; condition 3:10 nM soto-p₅/A02 tetramer (on streptavidin-DyLight650).

FIG. 55. Heatmap representation of single-point mutants in VH of RA_D11 that retained antigen binding (left column) or that lost binding (right column). The numbers indicate number of reads for a mutation of interest divided by the total number of reads for that position multiplied by 100. Condition 1:10 nM soto-p₇/A03; condition 2:10 nM soto-p7/A11; condition 3:10 nM soto-p₈/A02 tetramer (on streptavidin-DyLight650).

FIG. 56. Fab RA_D11 can specifically bind with high affinity to all four soto-pMHC targets, and it is minimally inhibited by the free inhibitor. The minimal inhibition of RA_D11 binding by free sotorasib can be a fundamental prerequisite for coadministration with the inhibitor.

FIGS. 57A-C. CryoEM structures of RA_D11 bound to multiple drug-peptide/MHC complexes elucidate the molecular basis of target recognition. RA_D11 recognizes soto-p/A03-A11 targets in a similar way, mainly interacting with the HLA surface and sotorasib (with a pocket formed by the interface of the Fab variable chains), while having minimal contacts with the rest of the peptide (p₇ or p₈).

FIGS. 58A and 58B. RA_D11 binds to the soto-pMHCs in a non-canonical manner, differently from typical TCR-pMHC engagement.

FIGS. 59A-C. RA_D11 can recognize both soto-p₇/A03 and soto-p₈/A03 in an almost identical way thanks to a different peptide accommodation of soto-p₈ (stretched) vs. soto-p₇ (bent) into the HLA-A*03 pocket. When soto-p₇ peptide is loaded by HLA-A*03 or A*11 different residues between the two HLAs result in a slightly different conformation of the Fab CDRs (in particular CDR-H3) when binding to the targets.

FIGS. 60A and 60B. RA_D11 can also bind to soto-p₅/A02, and accordingly RA_D11-based specific T-cell engager can selectively kill KRAS(G12C) cancer cells having the most diffused HLA-A*02 as well. RA_D11 binds soto-p₅/A02 in a non-univocal way, showing multiple possible orientations and have a reduced surface contract with the HLA with respect to soto-p/A03-A11 targets binding.

FIG. 61. Soto-p₅ is a much different peptide from soto-p₇₋₈ and must be presented in a distinct way.

FIG. 62. Establishing grid on the MHC backbone. The grid angles were set using the following α-carbons from amino acids of the MHC complex: origin set using was Met98; X-axis was set using Val28; Y-axis was set using Glu128; and the Y-axis was orthogonal. Incident angle was measured along the X-Y axis and back angle was measured along the Y-Z axis.

FIG. 63. Binding angles of soto-p₇/A03 and RA_D11 antibody. The angles were measured from axes to the center of the RA_D11 antibody (Met105 sulfur). The back angle, taken from the Y-Z axis, was measured as 64.7°. The incident angle, taken from the X-Y axis, was measured as 77.3°.

FIG. 64. Binding angles of soto-p₇/A11 and RA_D11 antibody. The angles were measured from axes to the center of the RA_D11 antibody (Met105 sulfur). The back angle, taken from the Y-Z axis, was measured as 67.2°. The incident angle, taken from the X-Y axis, was measured as 78.4°.

FIG. 65. Binding angles of soto-p₈/A03 and RA_D11 antibody. The angles were measured from axes to the center of the RA_D11 antibody (Met105 sulfur). The back angle, taken from the Y-Z axis, was measured as 65.1°. The incident angle, taken from the X-Y axis, was measured as 77.2°.

FIG. 66. Binding angles of a TCR-like antibody from PDB 3CHV. The angles were measured from axes to the center of the TCR-like antibody (Phe107 C4). The back angle, taken from the Y-Z axis, was measured as 92.5°. The incident angle, taken from the X-Y axis, was measured as 111.1°.

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

This disclosure includes every amino acid sequence described herein and all nucleotide sequences encoding the amino acid sequences. Every antibody sequence and antigen binding fragments of them are included. Polynucleotide and amino acid sequences having from 80-99% similarity, inclusive, and including all numbers and ranges of numbers there between, with the sequences provided herein, are included in the invention. All of the amino acid sequences described herein can include amino acid substitutions, such as conservative substitutions, that do not adversely affect the function of the protein that comprises the amino acid sequences. In this regard, the disclosure provides alternative residues for certain positions in described binding partner as described below. In certain examples, the alternative residues were identified by deep mutational scanning, which demonstrates binding functionality for each binding partner that contains the described amino acid change(s). The disclosure includes each binding partner with each alternative residue substituted for the original residue alone and in any combination with the described alternative residues. Thus, any binding partner described herein may have any single described residue change or a combination of described changes. Representative changes for particular antibodies are described the Tables. The changes may be in CDR1, CDR2, CDR3, and combinations thereof. The changes can also include amino acid insertions. The disclosure includes each amino acid sequence that is encompassed by the description of alternative amino acids by reference to a specific sequence identifier and those described in the aforementioned Tables.

As described above, the present disclosure provides antibodies and antigen binding fragments thereof (collectively “binding partners” and each individually a “binding partner”). The term “antibody” includes each binding partner format herein. The binding partners bind with specificity to a protein or fragment thereof, or a peptide provided in peptide form, that comprises a covalently attached molecule. The covalently attached molecule forms a peptide conjugate. A “peptide conjugate” as used herein means any protein or peptide that has been modified so that it is covalently conjugated to another molecule. The peptide conjugate is considered to be a novel antigen, i.e., a neoantigen. The other molecule that is covalently conjugated to the protein or peptide to form the peptide conjugate is not particularly limited, with the proviso that the other molecule is not an additional amino acid that is added to the described peptide conjugates. In embodiments, the molecule that is covalently conjugated to the protein or peptide has or had biological activity before conjugation, or it may be biologically inert before conjugation. In embodiments, the molecule is a drug, including but not necessarily limited to small molecule drugs. As used herein, the molecule that is covalently attached to a peptide to form peptide conjugate is referred to as a “targeted covalent inhibitor (TCI)” or as a “covalent drug.” In some cases, the targeted covalent inhibitor can be a molecule that inhibits an activity of a target protein and can be covalently attached to a peptide from the target protein. In some cases, the targeted covalent inhibitor may not inhibit an activity of a target protein, but can be covalently attached to the peptide from the target protein. Representative and non-limiting examples of drugs that covalently attach to a peptide or protein to form a peptide conjugate are described below. Peptide conjugates include but are not limited to covalently modified full length proteins and fragments thereof. Peptide conjugates include fragments of full length proteins that include a covalent modification and are produced, for example, by intracellular processing. In certain embodiments, a full length protein may be covalently modified within a cell and subsequently processed such that a peptide conjugate that is a fragment of the full length protein is produced. In an embodiment, the peptide conjugate comprises a fragment of a full-length protein. As described further below, the produced peptide conjugate may be displayed on a cell surface. The cell surface display of the peptide conjugate may be any form of cell surface display, including but not limited to by way of any receptor having an extracellular segment, or it may be displayed by way of any type of major histocompatibility complex (MHC) or human leukocyte antigen (HLA). Non-limiting examples of HLA types that display peptide conjugates, and to which the described binding partners bind with specificity, are described further below.

As used herein, the term “peptide conjugate/MHC complex” refers to a peptide conjugate comprising: a peptide and a chemical fragment of a targeted covalent inhibitor, presented by a major histocompatibility complex (MHC). For example, the peptide conjugate can be formed by the covalent reaction of a targeted covalent inhibitor with a residue (e.g., a cysteine residue) in a peptide. In some embodiments, the peptide conjugate is formed by the covalent reaction of AMG-510 with a KRAS^{G12C} peptide. In some embodiments, the peptide is externally introduced as a vaccine. In some embodiments, the peptide comprises a nucleophilic or an electrophilic residue. In some embodiments, the residue comprises cysteine, lysine, tyrosine, histidine, serine, arginine, or threonine. In an embodiment, the MHC is a human leukocyte antigen (HLA). In an embodiment, the HLA is HLA-A*01:01, HLA-A*02:01, HLA-A*03:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, or HLA-B*15:01.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., a peptide conjugate). CDR-H1, CDR-H2, and CDR-H3 denote the heavy chain CDRs, and CDR-L1, CDR-L2 and CDR-L3 denote the light chain CDRs.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul SF (1990) PNAS 87:2264-2268, modified as in Karlin S & Altschul SF (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the terms “free targeted covalent inhibitor” or “free drug” refer to a targeted covalent inhibitor that is not covalently linked to a protein or peptide. Once the targeted covalent inhibitor is covalently linked to a protein or peptide, the targeted covalent inhibitor can be referred as a portion or fragment of the free target covalent inhibitor or drug. For example, the protein or fragment thereof can be the chemical fragment that is bonded to the cysteine residue of the peptide upon covalent reaction of the free drug with the cysteine residue of the peptide. As used herein the term “KRAS^{G12C}” refers to the KRAS protein (UniProt Accession No. P01116) with a G12C mutation, i.e., a cysteine at amino acid position 12. As used herein the term “KRAS^{G12D}” refers to the KRAS protein (UniProt Accession No. P01116) with a G12D mutation, i.e., an aspartic acid at amino acid position 12. As used herein the term “KRAS^{G12R}” refers to the KRAS protein (UniProt Accession No. P01116) with a G12R mutation, i.e., an arginine at amino acid position 12. As used herein the term “KRAS^{G12S}” refers to the KRAS protein (UniProt Accession No. P01116) with a G12S mutation, i.e., a serine at amino acid position 12.

In embodiments, the binding partners preferentially bind to the protein or peptide or a complex comprising the protein or peptide when covalently bound to the peptide conjugate, relative to the same protein or peptide that is not bound to the drug. Accordingly, binding partners described herein either do not detectably bind, or bind with a lower affinity, to the same protein or fragment thereof in the absence of the covalently attached molecule. In embodiments, the binding partners bind to the protein or peptide comprising the covalently attached drug with an affinity that is 10-10,000 fold, including all numbers and ranges of numbers from 10-10,000, greater than the affinity for the protein or peptide that does not comprise the covalently bound molecule. In this regard, and without intending to be bound by any particular theory, it is considered that the presence of the covalently bound molecule contributes to the epitope to which the binding partners bind with specificity. Likewise, binding partners of this disclosure preferentially bind to the peptide conjugate relative to binding to the free drug. In embodiments, the binding partners bind to the peptide comprising the covalently attached drug (e.g., the peptide-conjugate/MHC complex) with an affinity that is 10-10,000 fold, including all numbers and ranges of numbers from 10-10,000, greater than the affinity for the free drug. In some embodiments, the interaction between the binding partner and the peptide-conjugate/MHC complex is not inhibited by the free drug. For example, the interaction between the binding partner and the peptide-conjugate/MHC complex is not inhibited by a 100×, 1,000×, 10,000×, 100,000× or more excess of the free drug.

In embodiments, the molecule that is covalently bound to form the peptide conjugate is a drug and may be any targeted covalent inhibitor (TCI), but the covalent drug need not necessarily inhibit the target peptide. In embodiments, the molecule reacts with a specific residue within the target protein. In embodiments, the molecule reacts at least in part with a segment of the protein or peptide that comprises a nucleophilic, or an electrophilic, residue. In embodiments, the segment of the protein or peptide to which the molecule reacts comprises any of Cys, Lys, Tyr, His, Ser, Thr, or Arg, the latter being described in Ziyang Zhang, Johannes Morstein, Andrew K. Ecker, Keelan Z. Guiley, and Kevan M. Shokat Journal of the American Chemical Society Article ASAP, DOI: 10.1021/jacs.2c05377, from which the disclosure is incorporated herein by reference. In embodiments, the protein or peptide comprises a selenocysteine. In embodiments, the targeted covalent inhibitor reacts with selenocysteine. In embodiments, the molecule reacts at least in part with a segment of the protein or peptide that comprises a wild type Cys, or a mutation of a residue to a Cys, and thus may be covalently attached by a so-called sulfur tether. In embodiments, the drug is any drug described in Ghosh A K, Samanta I, Mondal A, Liu W R. Covalent Inhibition in Drug Discovery. ChemMedChem. 2019; 14(9):889-906. doi:10.1002/cmdc.201900107, or in De Cesco, et al., European Journal of Medicinal Chemistry 138 (2017) 96e114, or in Bauer, RA, Drug Discovery Today, Volume 20, Number 9, September 2015, from which the disclosures of compounds that covalently modify protein targets is incorporated herein by reference. In non-limiting embodiments, any of said Cys, Lys, Tyr, Ser, Thr, Arg, and His amino acids are present in the protein or peptide to which the molecule binds because the gene encoding the wild type protein has been mutated to encode a protein that includes one or a combination of the described residues. In non-limiting embodiments, the molecule binds to a protein or peptide that is correlated with a disease or condition, such as a cancer, an autoimmune disease, or other disease or disorder that is treated with a targeted covalent inhibitor. In embodiments, the target (e.g., the protein or peptide to which the molecule covalently binds) is a receptor, including but not necessarily limited to any receptor having a catalytically active segment. In embodiments, the drug binds to an enzyme that is not necessarily a receptor, including but not limited to any kinase. In embodiments, a protein target comprises a receptor with one or more activating mutations, which promote ligand-independent enzyme activity.

In embodiments, the molecule targets and thus covalently binds to an amino acid sequence present within any of the following proteins and/or variants thereof, which may or may not comprise a mutation, such as a mutation that is related to a particular condition, including but not limited to any type of cancer. In embodiments, the protein is any protein described in Visscher M, et al., Covalent targeting of acquired cysteines in cancer. Curr Opin Chem Biol. 2016; 30:61-67. doi:10.1016/j.cbpa.2015.11.004, from which the description is incorporated herein by reference. Visscher et al. also teaches methods for identifying disease-associated mutated genes that introduces a Cys residue suitable for covalent modification. In embodiments, the protein is KRAS, Bruton's tyrosine kinase (BTK), any member of the epidermal growth factor receptor (EGFR) family, also referred to as the ERBB family, including but not limited to EGFR (ERBB1), HER2/NEU (ERBB2), HER3 (ERBB3), and HER4 (ERBB4); a fibroblast growth factor receptor (FGFR); the receptor kinase known in the art as MET, BRAF, a cyclin-dependent kinase (CDK); Acetyl Choline Esterase (ACHE); TP53, IDH1, GNAS, FBXW7, CTNNB1, DNMT3A, any cathepsin, including cathepsin B, C, F, H, K, L, O, S, V, W and X; any caspase; any protein involved in obesity, such as Pancreatic lipase and METAP2, or any Cancer Testis Antigen. In embodiments, the drug targets and therefore covalently binds to any viral protein, including but not limited to a polymerase, including any viral DNA polymerase, RNA polymerase, reverse transcriptase, or RNA-dependent RNA polymerase, or a viral protein that is required, for example, viral cell entry, or a protein encoded by any a transposable element. In embodiments, the drug targets EGFR and may be selected from PD168393, PF00299804 (dacomitinib), EKB569 (pelitinib), afatinib, WZ4002, osimertinib (formerly known as AZD9291), PF-06459988, nazartinib, naquotinib, olmutinib, avitinib, and rociletinib, neratinib, pyrotinib, poziotinib, and derivatives thereof. In embodiments, the drug targets Bruton's tyrosine kinase (BTK), and may be selected from ibrutinib, acalabrutinib, zanubrutinib, CHMFL-BTK-11, ONO/GS-405, PRN1008, and CC-292. In embodiments, the drug targets any p90 ribosomal S6 kinase (RSK), and may be selected from fluoromethylketone (FMK) and dimethyl fumarate. In embodiments, the drug targets any FGFR, and may be selected from FIIN-1, FIIN-2, FIIN-3, BGJ398, AZD4547, PRN1371, FGF401. In an embodiment, the targeted covalent inhibitor targets an E3 ligase, such as RNF4, HOIP, RSP5, SMURF1, E6AP, HUWE1, and NEDD4-1. In an embodiment, the targeted covalent inhibitor targets a DDB1- and CUL4-associated factor (DCAF), such as DCAF1 or DCAF15. In an embodiment, the targeted covalent inhibitor targets any cancer testis antigen, any endogenous retroviral protein, a long interspersed element-1 (LINE-1), or a short interspersed element (SINE). In an embodiment, the targeted covalent inhibitor targets a short interspersed element that is optionally Alu. In an embodiment, the targeted covalent inhibitor is iniparib, abiraterone, carfilzomib, afatinib, or neratinib.

In embodiments, the molecule that becomes covalently bound to form the peptide conjugate targets any RAS oncogene protein product, including but not necessarily limited to HRAS, NRAS, KRAS4A, and KRAS4B. The amino acid sequences of RAS proteins are known in the art, and residue numbering is identical for the relevant part of all RAS isotypes that are discussed in this disclosure for which the amino acid sequence is available from, for example, UniProt P01116, from which the amino acid sequence is incorporated herein as of the effective filing date of this application or patent. The G12 position is numbered according to the known amino acid sequence, regardless of whether or not the G12 is the twelfth amino acid in an express RAS peptide sequence of this disclosure.

In one embodiment, the molecule covalently binds to a KRAS protein or peptide that comprises a mutation. In embodiments, the mutation is at least one of KRAS residues 12, 13, or 61. Reference to any drug herein includes its name in capitalized and un-capitalized form.

In some embodiments, the drug targets a KRAS protein comprising a KRAS^{G12C} mutation. In some embodiments, the drug targets a KRAS protein comprising a KRAS^{G12D} mutation. In some embodiments, the drug targets a KRAS protein comprising a KRAS^{G12R} mutation. In some embodiments, the drug targets a KRAS protein comprising a KRAS^{G12S} mutation. In non-limiting embodiments, the drug that targets a KRAS protein is selected from AMG-510 (sotorasib), ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391, or derivatives thereof. In other embodiments, the drug that targets a KRAS protein is selected from beta-lactones G12Si-1, G12Si-2, G12Si-3, G12Si-4 and G12Si-5. In an embodiment the drug comprises a proteolysis targeting chimera (PROTAC) derivative of a covalent drug, a non-limiting description of which is available in doi:10.1021/acscentsci.0c00411, from which the description of PROTACs is incorporated herein by reference. In embodiments, the PROTAC is LC-1 or LC-2. In embodiments, the disclosure relates to an autophagy-mediated degrader, referred to as an AUTAC, as described in doi.org/10.1080/15548627.2020.1718362, from which the description of AUTACs is incorporated herein by reference.

The peptide conjugate described herein can be formed by the covalent reaction of a free targeted covalent inhibitor with a KRAS peptide. The peptide conjugate can be formed by the covalent reaction of a free targeted covalent inhibitor with a KRAS^{G12C} peptide, a KRAS^{G12D} peptide, a KRAS^{G12R} peptide, or a KRAS^{G12S}. The free targeted covalent inhibitor can be any free targeted covalent inhibitor described herein. For example, the free targeted covalent inhibitor can be osimertinib, ibrutinib, neratinib, AMG-510, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391, or derivatives thereof. In other embodiments, the free targeted covalent inhibitor can be beta-lactones G12Si-1, G12Si-2, G12Si-3, G12Si-4, or G12Si-5. (see Table R). In some cases, the peptide conjugate can be formed by the covalent reaction of AMG-510 with a KRAS^{G12C} peptide. The peptide can comprise or consist of the amino acid sequence of VVVGACGVGK, VVGACGVGK, or KLVVVGACGV.

The antigen binding domain of the polypeptide described herein or the binding partner described herein can bind to the peptide conjugate/MHC complex presented by different HLAs. For example, in certain embodiments, the antigen binding domain can have specificity to: (i) a peptide conjugate/MHC complex comprising VVVGACGVGK conjugated to a targeted covalent inhibitor or fragment thereof presented by HLA-A*11:01, (ii) a peptide conjugate/MHC complex comprising VVVGACGVGK conjugated to a targeted covalent inhibitor or fragment thereof presented by HLA-A*03:01, or (iii) both. In certain other embodiments, the antigen binding domain can have specificity to: (i) a peptide conjugate/MHC complex comprising VVGACGVGK conjugated to a targeted covalent inhibitor or fragment thereof presented by HLA-A*03:01, (ii) a peptide conjugate/MHC complex comprising VVVGACGVGK conjugated to a targeted covalent inhibitor or fragment thereof presented by HLA-A*03:01, or (iii) both. In other embodiments, the antigen binding domain can have specificity to: (i) a peptide conjugate/MHC complex comprising VVVGACGVGK conjugated to a targeted covalent inhibitor or fragment thereof presented by HLA-A*11:01, (ii) a peptide conjugate/MHC complex comprising VVGACGVGK conjugated to a targeted covalent inhibitor or fragment thereof presented by HLA-A*11:01, (iii) a peptide conjugate/MHC complex comprising VVGACGVGK conjugated to a targeted covalent inhibitor or fragment thereof presented by HLA-A*03:01, (iv) a peptide conjugate/MHC complex comprising VVVGACGVGK conjugated to a targeted covalent inhibitor or fragment thereof presented by HLA-A*03:01, (v) a peptide conjugate/MHC complex comprising KLVVVGACGV conjugated to a targeted covalent inhibitor or fragment thereof presented by HLA-A*02:01, or (vi) any combination of (i)-(v) (e.g., (i) and (ii); or (i), (ii), and (iv)), or (vii) all of (i)-(v). In certain specific embodiments, the polypeptide binds to: (i) a peptide conjugate/HLA-A*11:01 MHC complex containing a peptide consisting of the amino acid sequence VVGACGVGK, (ii) a peptide conjugate/HLA-A*11:01 MHC complex containing a peptide consisting of the amino acid sequence VVVGACGVGK, (iii) a peptide conjugate/HLA-A*03:01 MHC complex containing a peptide consisting of the amino acid sequence VVGACGVGK, (iv) a peptide conjugate/HLA-A*03:01 MHC complex containing a peptide consisting of the amino acid sequence VVVGACGVGK, and/or (v) a peptide conjugate/HLA-A*02:01 MHC complex containing a peptide consisting of the amino acid sequence KLVVVGACGV. In still other embodiments, the polypeptide binds to: (i) a peptide conjugate/HLA-A*03:01 MHC complex containing a peptide consisting of the amino acid sequence VVVGACGVGK and a peptide conjugate/HLA-A*11:01 MHC complex containing a peptide consisting of the amino acid sequence VVVGACGVGK; (ii) a peptide conjugate/HLA-A*03:01 MHC complex containing a peptide consisting of the amino acid sequence VVGACGVGK and a peptide conjugate/HLA-A*03:01 MHC complex containing a peptide consisting of the amino acid sequence VVVGACGVGK; (iii) a peptide conjugate/HLA-A*02:01 MHC complex containing a peptide consisting of the amino acid sequence KLVVVGACGV; or any combination of (i)-(iii) (e.g., (i) and (ii); or (i) and (iii)); or all of (i)-(iii). The covalent inhibitor that targets the peptide conjugate/MHC complex can have a chemical structure comprising C-R1, where C is a chemical fragment linked to R1 of any compound illustrated below.

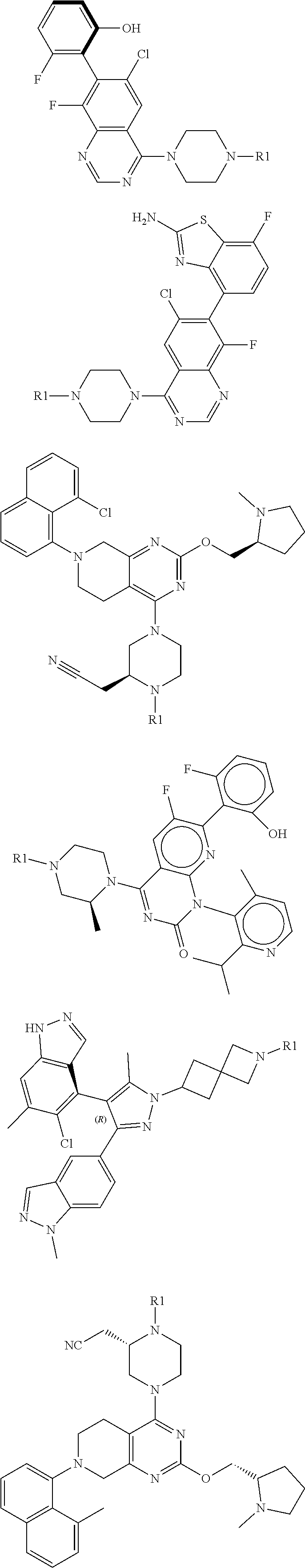

R1 of the C-R1 chemical structure of the covalent inhibitor can be any of the structures illustrated below.

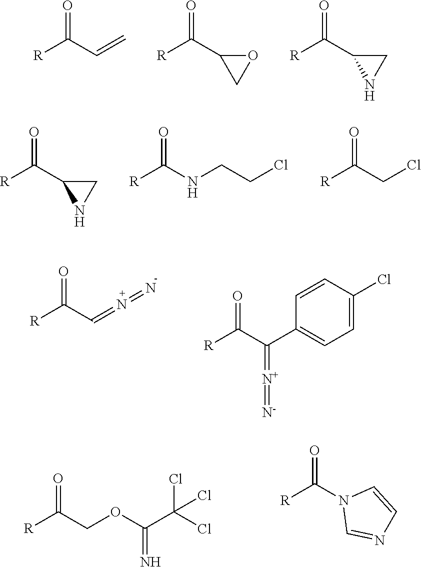

The targeted covalent inhibitor with the structure comprising C-R1 can form a covalent bond to several different amino acid residues on the peptide. For example, the targeted covalent inhibitor can form a covalent bond to the cysteine residue in a peptide comprising the amino acid sequence of VVVGACGVGK, VVGACGVGK, or KLVVVGACGV. In other embodiments, the targeted covalent inhibitor can form a covalent bond to the aspartic acid residue, the serine residue, or the arginine residue in a peptide comprising the amino acid sequence of VVVGADGVGK, VVGADGVGK, or KLVVVGADGV. In some embodiments, the antigen binding domain of the polypeptide recognizes the C portion of the targeted covalent inhibitor with the structure comprising C-R1 of the peptide conjugate/MHC complex. In certain other embodiments, the antigen binding domain of the polypeptide recognizes C portion but not R1 portion of the targeted covalent inhibitor of the peptide conjugate/MHC complex.

Small molecules (e.g., targeted covalent inhibitors) having an electrophilic warhead group can undergo covalent reaction with a cysteine residue of a peptide to form a peptide-small molecule conjugate. This type of reaction is illustrated in the following scheme for AMG-510 (sotorasib).

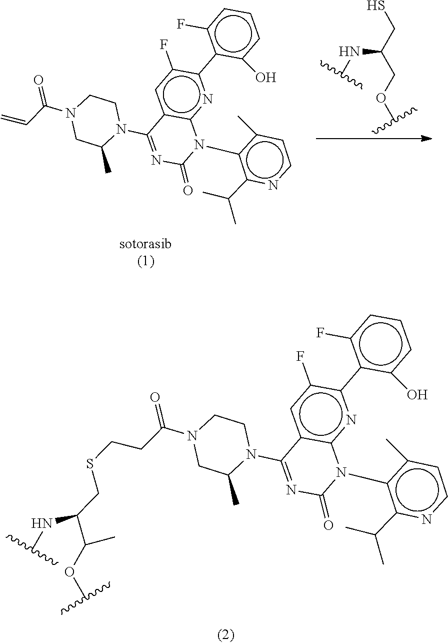

The table below lists targeted covalent inhibitors, along with the chemical structure of the fragment (“chemical fragment”) that is bonded to the cysteine residue of the peptide upon covalent reaction of the drug with the cysteine. In an embodiment, the binding partners disclosed herein bind to a peptide conjugate/MHC complex comprising a peptide conjugated to a chemical fragment in Table K below and an MHC.

TABLE K
Structures of targeted covalent inhibitors and the chemical fragments after
covalent reaction with a cysteine residue.

| | | | Chemical Fragment that is |
|---|---|---|---|
| | | | Covalently Attached to Cysteine |
| | | | Residue of Peptide Upon Reaction |
| No | Drug Name | Drug Structure | of Drug with Peptide |
| 1 | sotorasib | 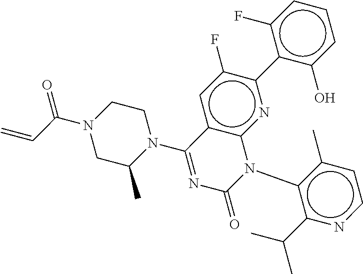 | 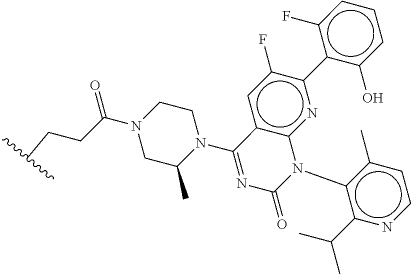 |
| | | | Compound 1 |
| 2 | osimertinib | 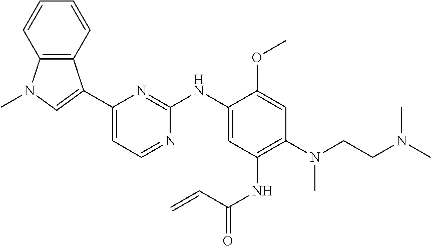 | 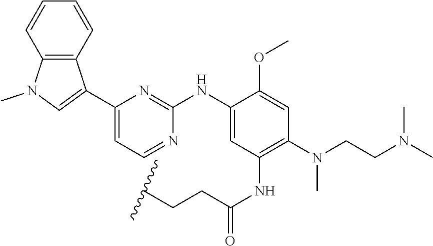 |
| | | | Compound 2 |
| 3 | ibrutinib | 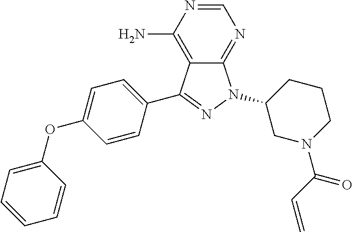 [SMILES: C=CC(=O)N1CCC[C@@H](n2nc(-c3ccc(Oc4ccccc4)cc3)c3c(N)ncnc32)C1] | 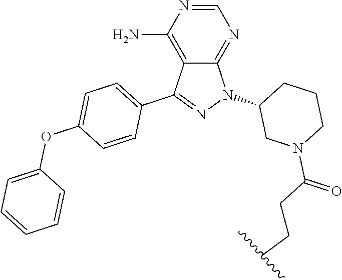 [SMILES: *CCC(=O)N1CCC[C@@H](n2nc(-c3ccc(Oc4ccccc4)cc3)c3c(N)ncnc32)C1] |
| | | | Compound 3 |
| 4 | ARS-1620 | 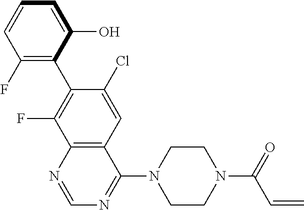 | 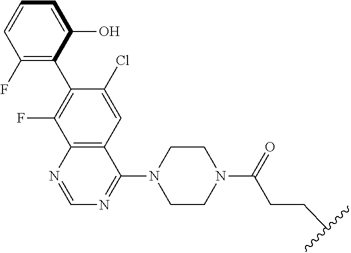 |
| | | | Compound 4 |
| 5 | adagrasib (MRTX849) | 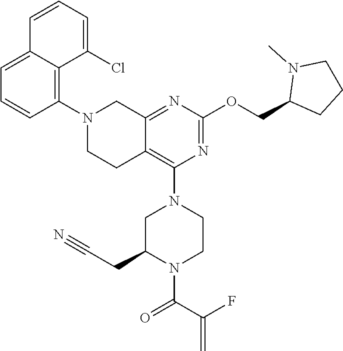 [SMILES: C=C(F)C(=O)N1CCN(c2nc(OC[C@@H]3CCCN3C)nc3c2CCN(c2cccc4cccc(Cl)c24)C3)C[C@@H]1CC#N] | 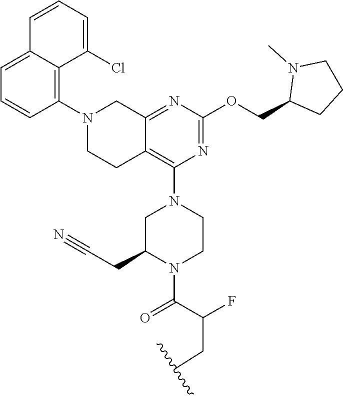 [SMILES: *CC(F)C(=O)N1CCN(c2nc(OC[C@@H]3CCCN3C)nc3c2CCN(c2cccc4cccc(Cl)c24)C3)C[C@@H]1CC#N] |
| | | | Compound 5 |
| 6 | JDQ443 | 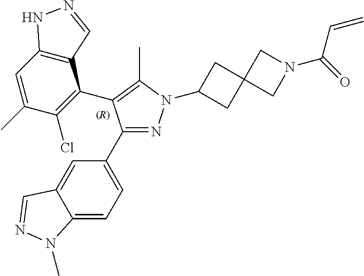 | 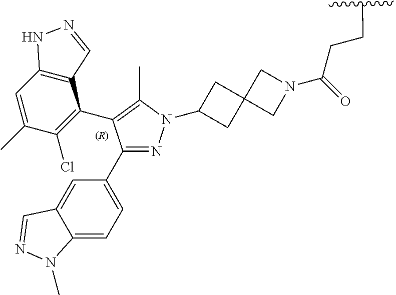 |
| | | | Compound 6 |
| 7 | LY3499446 | 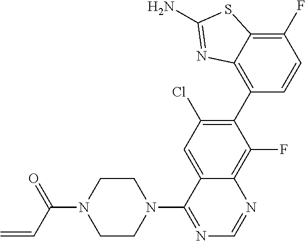 [SMILES: C=CC(=O)N1CCN(c2ncnc3c(F)c(-c4ccc(F)c5sc(N)nc45)c(Cl)cc23)CC1] | 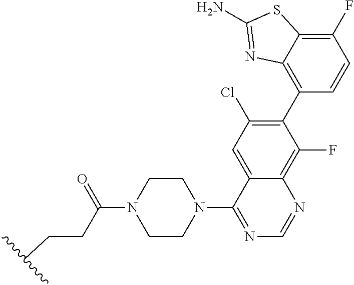 [SMILES: *CCC(=O)N1CCN(c2ncnc3c(F)c(-c4ccc(F)c5sc(N)nc45)c(Cl)cc23)CC1] |
| | | | Compound 7 |
| 8 | MRTX- 1257 | 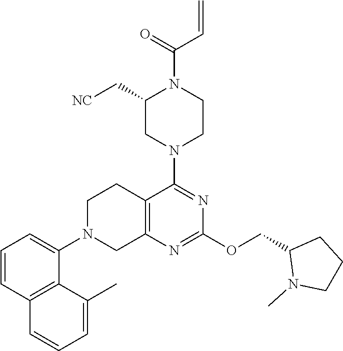 | 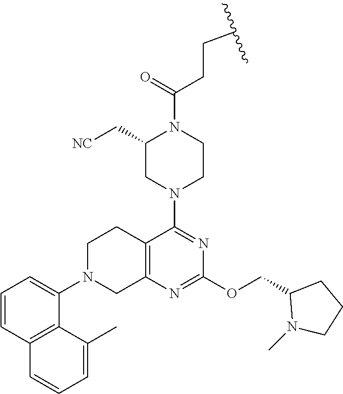 |
| | | MRTX1257 | Compound 8 |

TABLE R
Structures of exemplary targeted covalent inhibitors targeting G12S

| | |
|---|---|
| beta- lactone G12Si-1 | 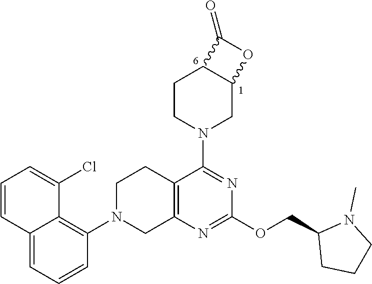 [SMILES: CN1CCC[C@H]1COc1nc2c(c(N3CCC4C(=O)OC4C3)n1)CCN(c1cccc3cccc(Cl)c13)C2] |
| beta- lactone G12Si-2 | 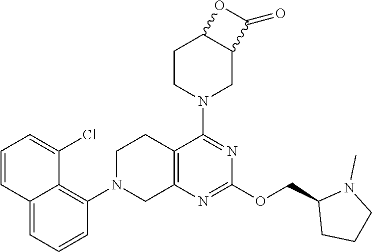 [SMILES: CN1CCC[C@H]1COc1nc2c(c(N3CCC4OC(=O)C4C3)n1)CCN(c1cccc3cccc(Cl)c13)C2] |
| beta- lactone G12Si-3 | 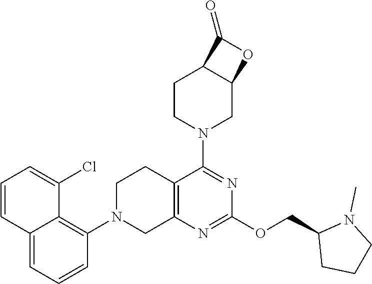 [SMILES: CN1CCC[C@H]1COc1nc2c(c(N3CC[C@H]4C(=O)O[C@H]4C3)n1)CCN(c1cccc3cccc(Cl)c13)C2] |
| beta- lactone G12Si-4 | 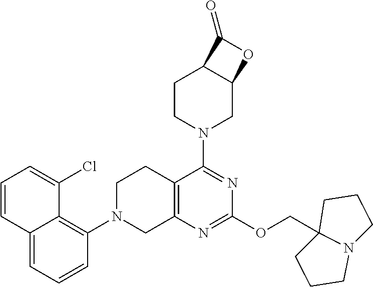 [SMILES: O=C1O[C@H]2CN(c3nc(OCC45CCCN4CCC5)nc4c3CCN(c3cccc5cccc(Cl)c35)C4)CC[C@@H]12] |
| beta- lactone G12Si-5 | 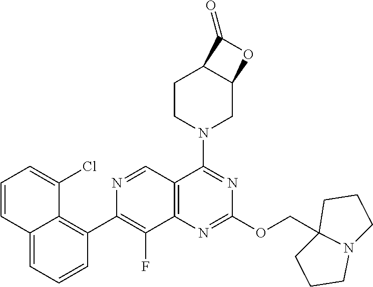 [SMILES: O=C1O[C@H]2CN(c3nc(OCC45CCCN4CCC5)nc4c(F)c(-c5cccc6cccc(Cl)c56)ncc34)CC[C@@H]12] |

In a non-limiting embodiment, the binding partner binds with specificity to a site comprising a neoantigen that includes a covalently linked small molecule drug or other covalently linked molecule as a component of an antigen in a specific MHC context. In an aspect, provided herein is a binding partner that specifically binds to a peptide conjugate/MHC complex, wherein the peptide conjugate is formed by the covalent reaction of a targeted covalent inhibitor with a peptide. In an embodiment, the binding partner binds to the peptide conjugate/MHC complex with a greater affinity than to the peptide or free targeted covalent inhibitor.

In an embodiment, the affinity of the binding partner for the peptide conjugate/MHC complex is 100-10,000 times greater than the affinity of the binding partner for the peptide or free targeted covalent inhibitor. In an embodiment, the affinity of the binding partner for the peptide conjugate/MHC complex is at least 100 times, at least 200 times, at least 300 times, at least 400 times, at least 500 times, at least 600 times, at least 700 times, at least 800 times, at least 900 times, at least 1,000 times, at least 2,500 times, at least 5,000 times, or at least 10,000 times greater than the affinity of the binding partner to the free targeted covalent inhibitor or the free peptide conjugate. In some embodiments, the affinity of the binding partner to the free drug is reported as IC₅₀. In some embodiments, the affinity of the binding partner to the free drug is reported as an IC₅₀ of more than 1 μM. In some embodiments, the affinity of the binding partner to the free drug is reported as an IC₅₀ of more than 5 μM. In some embodiments, the affinity of the binding partner to the free drug is reported as an IC₅₀ of more than 10 μM. In some embodiments, the affinity of the binding partner to the free drug is reported as an IC₅₀ of more than 15 μM. In some embodiments, the affinity of the binding partner to the free drug is reported as an IC₅₀ of more than 20 μM. In some embodiments, the affinity of the binding partner to the free drug is reported as an IC₅₀ of more than 30 μM. In some embodiments, the affinity of the binding partner to the free drug is reported as an IC₅₀ of more than 40 μM. In some embodiments, the affinity of the binding partner to the free drug is reported as an IC₅₀ of more than 50 μM. In some embodiments, the affinity of the binding partner to the free drug is reported as an IC₅₀ of more than 75 μM. In some embodiments, the affinity of the binding partner to the free drug is reported as an IC₅₀ of more than 100 μM. In some embodiments, the affinity of the binding partner to the free drug is reported as an IC₅₀ of more than 200 μM.

In some embodiments, the antigen-binding domain does not detectably bind to the free targeted covalent inhibitor. In some embodiments, the antigen-binding domain binds to the free targeted covalent inhibitor with an IC₅₀ of at least about 50 nM. In some embodiments, the antigen-binding domain does not bind to the free targeted covalent inhibitor with an IC₅₀ of lower than about 50 nM. The antigen-binding domain can bind to the free targeted covalent inhibitor with an IC₅₀ of at least about 5 nM. The antigen-binding domain can bind to the free targeted covalent inhibitor with an IC₅₀ of at least about 25 nM. The antigen-binding domain can bind to the free targeted covalent inhibitor with an IC₅₀ of at least about 40 nM. The antigen-binding domain can bind to the free targeted covalent inhibitor with an IC₅₀ of at least about 60 nM. The antigen-binding domain can bind to the free targeted covalent inhibitor with an IC₅₀ of at least about 75 nM. The antigen-binding domain can bind to the free targeted covalent inhibitor with an IC₅₀ of at least about 100 nM. The antigen-binding domain can bind to the free targeted covalent inhibitor with an IC₅₀ of at least about 250 nM. The antigen-binding domain can bind to the free targeted covalent inhibitor with an IC₅₀ of at least about 500 nM. The antigen-binding domain can bind to the free targeted covalent inhibitor with an IC₅₀ of at least about 750 nM. The antigen-binding domain can bind to the free targeted covalent inhibitor with an IC₅₀ of at least about 1000 nM. The antigen-binding domain can bind to the free targeted covalent inhibitor with an IC₅₀ of at least about 2 μM. The antigen-binding domain can bind to the free targeted covalent inhibitor with an IC₅₀ of at least about 5 μM. The antigen-binding domain can bind to the free targeted covalent inhibitor with an IC₅₀ of at least about 10 μM. The antigen-binding domain can bind to the free targeted covalent inhibitor with an IC₅₀ of at least about 15 μM. The antigen-binding domain can bind to the free targeted covalent inhibitor with an IC₅₀ of at least about 20 μM. The antigen-binding domain can bind to the free targeted covalent inhibitor with an IC₅₀ of at least about 25 μM. The antigen-binding domain can bind to the free targeted covalent inhibitor with an IC₅₀ of at least about 50 μM.

In some embodiments, the antigen-binding domain may bind to the free targeted covalent inhibitor with an IC₅₀ of less than about 50 nM. The antigen-binding domain may bind to the free targeted covalent inhibitor with an IC₅₀ of less than about 5 nM. The antigen-binding domain may bind to the free targeted covalent inhibitor with an IC₅₀ of less than about 25 nM. The antigen-binding domain may bind to the free targeted covalent inhibitor with an IC₅₀ of less than about 40 nM. The antigen-binding domain may bind to the free targeted covalent inhibitor with an IC₅₀ of less than about 60 nM. The antigen-binding domain may bind to the free targeted covalent inhibitor with an IC₅₀ of less than about 75 nM. The antigen-binding domain may bind to the free targeted covalent inhibitor with an IC₅₀ of less than about 100 nM. The antigen-binding domain may bind to the free targeted covalent inhibitor with an IC₅₀ of less than about 250 nM. The antigen-binding domain may bind to the free targeted covalent inhibitor with an IC₅₀ of less than about 500 nM. The antigen-binding domain may bind to the free targeted covalent inhibitor with an IC₅₀ of less than about 750 nM. The antigen-binding domain may bind to the free targeted covalent inhibitor with an IC₅₀ of less than about 1000 nM. The antigen-binding domain may bind to the free targeted covalent inhibitor with an IC₅₀ of less than about 2 μM. The antigen-binding domain may bind to the free targeted covalent inhibitor with an IC₅₀ of less than about 5 μM. The antigen-binding domain may bind to the free targeted covalent inhibitor with an IC₅₀ of less than about 10 μM. The antigen-binding domain may bind to the free targeted covalent inhibitor with an IC₅₀ of less than about 15 μM. The antigen-binding domain may bind to the free targeted covalent inhibitor with an IC₅₀ of less than about 20 μM. The antigen-binding domain may bind to the free targeted covalent inhibitor with an IC₅₀ of less than about 25 μM. The antigen-binding domain may bind to the free targeted covalent inhibitor with an IC₅₀ of less than about 50 μM.

The binding partner described herein may not bind to the peptide/MHC complex without the conjugate (e.g., the targeted covalent inhibitor). In some cases, the affinity of the binding partner to the peptide conjugate/MHC complex is reported as a dissociation constant (K_{D}) from 10 pM to 50 nM. In some cases, the affinity of the binding partner to the peptide conjugate/MHC complex is reported as a dissociation constant (K_{D}) from 1 pM to 10 pM. In some cases, the affinity of the binding partner to the peptide conjugate/MHC complex is reported as a dissociation constant (K_{D}) from 0.01 pM to 10 pM. In some cases, the affinity of the binding partner to the peptide conjugate/MHC complex is reported as a dissociation constant (K_{D}) from 0.1 nM to 1 nM. In some cases, the affinity of the binding partner to the peptide conjugate/MHC complex is reported as a dissociation constant (K_{D}) from 1 nM to 2 nM. In some cases, the affinity of the binding partner to the peptide conjugate/MHC complex is reported as a dissociation constant (K_{D}) from 2 nM to 5 nM. In some cases, the affinity of the binding partner to the peptide conjugate/MHC complex is reported as a dissociation constant (K_{D}) of equal to less than about 5 nM. In some cases, the affinity of the binding partner to the peptide conjugate/MHC complex is reported as a dissociation constant (K_{D}) of equal to less than about 4 nM. In some cases, the affinity of the binding partner to the peptide conjugate/MHC complex is reported as a dissociation constant (K_{D}) of equal to less than about 3 nM. In some cases, the affinity of the binding partner to the peptide conjugate/MHC complex is reported as a dissociation constant (K_{D}) of equal to less than about 2 nM. In some cases, the affinity of the binding partner to the peptide conjugate/MHC complex is reported as a dissociation constant (K_{D}) of equal to less than about 1 nM. In some cases, the affinity of the binding partner to the peptide conjugate/MHC complex is reported as a dissociation constant (K_{D}) of equal to less than about 0.1 nM. In some cases, the affinity of the binding partner to the peptide conjugate/MHC complex is reported as a dissociation constant (K_{D}) of about equal to less than 0.01 nM. In some cases, the affinity of the binding partner to the peptide conjugate/MHC complex is reported as a dissociation constant (K_{D}) of equal to less than about 1 pM. In some cases, the affinity of the binding partner to the peptide conjugate/MHC complex is reported as a dissociation constant (K_{D}) of equal to less than about 0.1 pM.

In some embodiments, the affinity of the binding partner to the peptide conjugate/MHC complex is reported as IC₅₀ or EC₅₀. In some embodiments, the affinity of the binding partner to the peptide conjugate/MHC complex is reported as an IC₅₀ of less than about 1 μm. In some embodiments, the affinity of the binding partner to the peptide conjugate/MHC complex is reported as an IC₅₀ of less than about 10 μm. In some embodiments, the affinity of the binding partner to the peptide conjugate/MHC complex is reported as an IC₅₀ of less than about 100 μm. In some embodiments, the affinity of the binding partner to the peptide conjugate/MHC complex is reported as an IC₅₀ of less than about 1000 μm. In some embodiments, the affinity of the binding partner to the peptide conjugate/MHC complex is reported as an IC₅₀ of less than about 1 nm. In some embodiments, the affinity of the binding partner to the peptide conjugate/MHC complex is reported as an IC₅₀ of less than about 10 nm. In some embodiments, the affinity of the binding partner to the peptide conjugate/MHC complex is reported as an IC₅₀ of less than about 100 nm. In some embodiments, the affinity of the binding partner to the peptide conjugate/MHC complex is reported as an IC₅₀ of less than about 1000 nm. In some embodiments, the affinity of the binding partner to the peptide conjugate/MHC complex is reported as an IC₅₀ of less than about 1 μM.

In some embodiments, the binding partner does not detectably bind to a complex of a peptide with a MHC molecule, wherein the peptide is not covalently bound to a non-peptide molecule. In some embodiments, the binding partner binds at less than 10× affinity to a complex of a peptide with a MHC molecule, wherein the peptide is not covalently bound to a non-peptide molecule. In some embodiments, the binding partner binds at less than 100× affinity to a complex of a peptide with a MHC molecule, wherein the peptide is not covalently bound to a non-peptide molecule. In some embodiments, the binding partner binds at less than 1,000× affinity to a complex of a peptide with a MHC molecule, wherein the peptide is not covalently bound to a non-peptide molecule.

In some embodiments, the binding partner can be a polypeptide. In some embodiments, the polypeptide can have an antigen-binding domain. The antigen-binding domain can bind to the peptide conjugate/MHC complex with a dissociation constant (K_{D}) of at most about 50 nM, at most about 40 nM, at most about 30 nM, at most about 20 nM, at most about 10 nM, at most about 1 nM, at most about 0.1 nM, at most about 10 pM, or at most about 1 pM. The antigen-binding domain can bind to the peptide conjugate/MHC complex with a dissociation constant (K_{D}) from about 0.01 nM to about 20 nM.

The antigen-binding domain can bind to the free targeted covalent inhibitor with a dissociation constant (K_{D}) that is higher than a K_{D} of the antibody or the antigen-binding fragment to the peptide conjugate/MHC complex. In some cases, the antigen-binding domain can bind to the free targeted covalent inhibitor with a dissociation constant (K_{D}) of at least about 100 nM, at least about 200 nM, at least about 300 nM, at least about 400 nM, at least about 500 nM, at least about 1 μM, at least about 10 μM, at least about 20 μM, at least about 30 μM, at least about 40 μM, at least about 50 M, or at least 100 μM. The antigen-binding domain can bind to the free targeted covalent inhibitor with a dissociation constant (K_{D}) of at least about 2, 3, 4, 5, 10, 20, 50, 100, 200, 500, 1,000, 2,000, 5,000, 10,000, 20,000, 50,000, 100,000 times more than a K_{D} of the antigen-binding domain binding to the peptide conjugate/MHC complex. The antigen-binding domain can bind to the free peptide conjugate with a dissociation constant (K_{D}) that is higher than a K_{D} of the antibody or the antigen-binding fragment to the peptide conjugate/MHC complex. The antigen-binding domain can bind to the free peptide conjugate with a dissociation constant (K_{D}) of more than about 100 nM, more than about 200 nM, more than about 300 nM, more than about 400 nM, more than about 500 nM, more than 1 μM, more than 10 μM, more than 20 μM, more than 30 μM, more than 40 μM, more than 50 μM, or more than 100 μM. The antigen-binding domain can bind to the free peptide conjugate with a dissociation constant (K_{D}) at least about 10, 100, 10,000, or 100,000 times more than a K_{D} of the antigen-binding domain binding to the peptide conjugate/MHC complex. The antigen-binding domain can bind to the free peptide conjugate with a dissociation constant (K_{D}) that is at least 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 times more than a K_{D} of the antibody or the antigen-binding fragment to the peptide conjugate/MHC complex. In some embodiments, the antigen-binding domain binds to the free peptide conjugate with a dissociation constant (K_{D}) that is at least 2.5 times more than a K_{D} of the antibody or the antigen-binding fragment to the peptide conjugate/MHC complex. The antigen-binding domain can bind to the peptide conjugate/MHC complex with an affinity that is at least 100 times, at least 200 times, at least 300 times, at least 400 times, at least 500 times, at least 600 times, at least 700 times, at least 800 times, at least 900 times, at least 1,000 times, at least 2,500 times, at least 5,000 times, or at least 10,000 times greater than the affinity of antigen-binding domain to the free targeted covalent inhibitor or the free peptide conjugate. The MHC can be HLA-A02:01. The MHC can be HLA-A03:01.

In an embodiment, the MHC is a human leukocyte antigen (HLA). In an embodiment, the HLA is any HLA found in the Allele Frequency Net Database (allelefrequencies.net). In an embodiment, the HLA is HLA-A*02:01, HLA-A*03:01, HLA-A*01:01, HLA-A*02:01, HLA-A*03:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, HLA-B*15:01, and/or HLA-A*11:01.

In an aspect, provided herein is a cell-free peptide conjugate/MHC complex comprising: (a) a peptide conjugate formed by the covalent reaction of a targeted covalent inhibitor with a peptide; and (b) an MHC. In an embodiment, the peptide comprises a segment of RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}), EGFR, BTK, HER2/NEU (ERBB2), HER3 (ERBB3), HER4 (ERBB4), MET (HGFR); FGFR, CDK, Acetylcholine Esterase (ACHE), p90 ribosomal S6 kinase (RSK), TP53, IDH1, GNAS, FBXW7, CTNNB1, DNMT3A, cathepsin B, cathepsin C, cathepsin F, cathepsin H, cathepsin K, cathepsin L, cathepsin O, cathepsin S, cathepsin V, cathepsin W, cathepsin X, a caspase, pancreatic lipase, METAP2, any cancer testis antigen, any endogenous retroviral protein, a long interspersed element-1 (LINE-1), or a short interspersed element (SINE). In an embodiment, the short interspersed element is Alu. In an embodiment, the targeted covalent inhibitor is osimertinib, ibrutinib, neratinib, AMG-510, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. Examples of additional compounds that can covalently target a KRAS peptide containing a G12C mutation can be found in Internal Application No. PCT/IB2019/050993, Internal Application No. PCT/EP2018/083853, and U.S. Application No. U.S. Ser. No. 16/917,128, each of which is incorporated herein by reference in its entirety. In other embodiments, the targeted covalent inhibitor is selected from beta-lactones G12Si-1, G12Si-2, G12Si-3, G12Si-4 and G12Si-5.

In an aspect, provided herein is a cell free peptide conjugate/MHC complex comprising: (a) a compound selected from the group consisting of compounds 1 and 4-8,

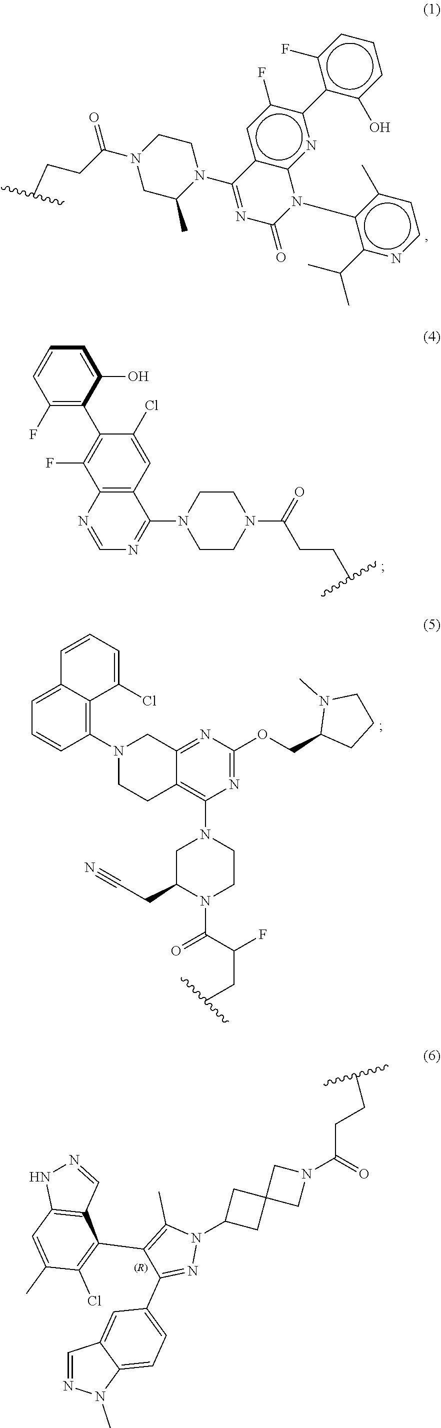

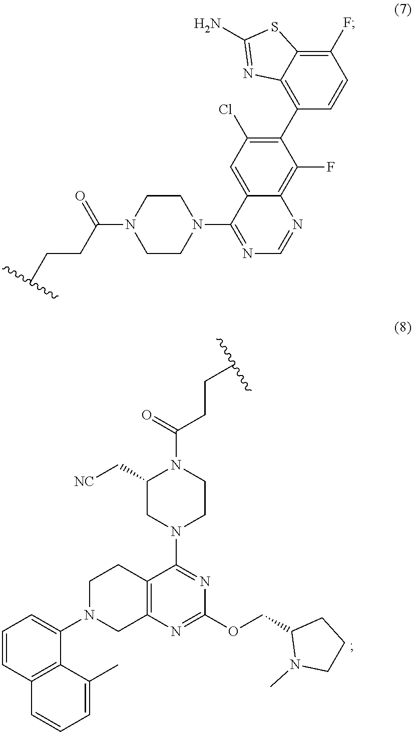

covalently bonded to a cysteine residue in a peptide comprising the amino acid sequence of VVVGACGVGK, VVGACGVGK or KLVVVGACGV; and (b) an MHC.

In an aspect, provided herein is a cell free peptide conjugate/MHC complex comprising: (a) compound 2:

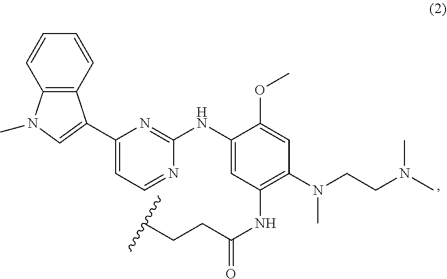

covalently bonded to the cysteine residue in a peptide comprising the amino acid sequence of QLMPFGCLL, LMPFGCLLDY, or MPFGCLLDY; and (b) an MHC.

In an aspect, provided herein is a cell-free peptide conjugate/MHC complex comprising: (a) compound 3:

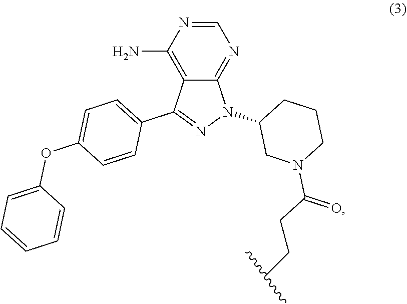 [SMILES: *CCC(=O)N1CCC[C@@H](n2nc(-c3ccc(Oc4ccccc4)cc3)c3c(N)ncnc32)C1]

covalently bonded to the cysteine residue in a peptide comprising the amino acid sequence of YMANGCLLNY; and (b) an MHC.

In an embodiment the MHC is an HLA, optionally wherein the HLA is HLA-A*02:01, HLA-A*03:01, HLA-A*01:01, HLA-A*02:01, HLA-A*03:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, HLA-B*15:01, or HLA-A*11:01.

In an aspect, provided herein is a binding partner that specifically binds to a conjugate formed by the covalent reaction of AMG-510 with a KRAS^{G12C} peptide presented by an HLA, wherein the binding partner comprises a heavy chain variable region (VH) and a light chain variable region (VL), wherein
(a) the VH comprises:
   (i) a CDR-H1 comprising the amino acid sequence of DYSIH, or a variant thereof comprising 1-5 amino acid changes;
   (ii) a CDR-H2 comprising the amino acid sequence of SISSSSGSTSYADSVKG, or a variant thereof comprising 1-5 amino acid changes; and/or
   (iii) a CDR-H3 comprising the amino acid sequence of GX₁WX₂X₃AMDY, wherein X₁ is G, R, H, S, or K, X₂ is Y or I, and X₃ is P or A; and/or
(b) the VL comprises:
   (i) a CDR-L1 comprising the amino acid sequence of RASQSVSSAVA, or a variant thereof comprising 1-5 amino acid changes;
   (ii) a CDR-L2 comprising the amino acid sequence of SASSLYS, or a variant thereof comprising 1-5 amino acid changes; and/or
   (iii) a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

The CDR sequences of exemplary antibodies that bind a conjugate formed by the covalent reaction of AMG-510 with a KRAS(G12C) peptide presented on HLA-A*03:01 and HLA-A*11:01 are provided in Table G and Table H below.

TABLE G
Heavy chain CDR sequences of exemplary antibodies
that bind a conjugate formed by the covalent
reaction of AMG-510 with a KRAS(G12C) peptide
presented on HLA-A*03: 01 and HLA-A*11: 01.

| Antibody | CDR-H1 | CDR-H2 | CDR-H3 |
|---|---|---|---|
| AMRA3-7D | DYSIH | SISSSSGSTSYADSVKG | GGWYPAMDY |
| RA_D01 | DYSIH | SISSSSGSTSYADSVKG | GGWIAAMDY |
| RA_D02 | DYSIH | SISSSSGSTSYADSVKG | GRWIPAMDY |
| RA_D03 | DYSIH | SISSSSGSTSYADSVKG | GGWIAAMDY |
| RA_D04 | DYSIH | SISSSSGSTSYADSVKG | GRWIPAMDY |
| RA_D06 | DYSIH | SISSSSGSTSYADSVKG | GHWIAAMDY |
| RA_D07 | DYSIH | SISSSSGSTSYADSVKG | GRWIPAMDY |
| RA_D08 | DYSIH | SISSSSGSTSYADSVKG | GGWIAAMDY |
| RA_D09 | DYSIH | SISSSSGSTSYADSVKG | GSWIAAMDY |
| RA_D11 | DYSIH | SISSSSGSTSYADSVKG | GGWIAAMDY |
| RA_D12 | DYSIH | SISSSSGSTSYADSVKG | GRWIPAMDY |
| RA_D13 | DYSIH | SISSSSGSTSYADSVKG | GKWIPAMDY |
| RA_D14 | DYSIH | SISSSSGSTSYADSVKG | GGWIAAMDY |
| RA_D16 | DYSIH | SISSSSGSTSYADSVKG | GRWIPAMDY |
| RA_D18 | DYSIH | SISSSSGSTSYADSVKG | GRWIPAMDY |
| RA_D19 | DYSIH | SISSSSGSTSYADSVKG | GRWIPAMDY |
| RA_D20 | DYSIH | SISSSSGSTSYADSVKG | GRWIPAMDY |
| RA_D21 | DYSIH | SISSSSGSTSYADSVKG | GRWIPAMDY |
| RA_D23 | DYSIH | SISSSSGSTSYADSVKG | GHWIAAMDY |
| RA_D24 | DYSIH | SISSSSGSTSYADSVKG | GRWIPAMDY |

TABLE H
Light chain CDR sequences of exemplary antibodies
that bind a conjugate formed by the covalent
reaction of AMG-510 with a KRAS(G12C) peptide
presented on HLA-A *03: 01 and HLA-A*11: 01.

| Antibody | CDR-L1 | CDR-L2 | CDR-L3 |
|---|---|---|---|
| AMRA3-7D | RASQSVSSAVA | SASSLYS | QQISYVKKLIT |
| RA_D01 | RASQSVSSAVA | SASSLYS | QQASYVRRTIT |
| RA_D02 | RASQSVSSAVA | SASSLYS | QQPSYVRRKIT |
| RA_D03 | RASQSVSSAVA | SASSLYS | QQASYVARKIT |
| RA_D04 | RASQSVSSAVA | SASSLYS | QQVSYVARRIT |
| RA_D06 | RASQSVSSAVA | SASSLYS | QQASYVKRLIT |
| RA_D07 | RASQSVSSAVA | SASSLYS | QQASYVRRLIT |
| RA_D08 | RASQSVSSAVA | SASSLYS | QQASYVRRVIT |
| RA_D09 | RASQSVSSAVA | SASSLYS | QQASYVRRTIT |
| RA_D11 | RASQSVSSAVA | SASSLYS | QQASYVRKTIT |
| RA_D12 | RASQSVSSAVA | SASSLYS | QQASYVAKTIT |
| RA_D13 | RASQSVSSAVA | SASSLYS | QQSSYVRRKIT |
| RA_D14 | RASQSVSSAVA | SASSLYS | QQASYVRKAIT |
| RA_D16 | RASQSVSSAVA | SASSLYS | QQASYVRRAIT |
| RA_D18 | RASQSVSSAVA | SASSLYS | QQSSYVKRTIT |
| RA_D19 | RASQSVSSAVA | SASSLYS | QQPSYVRKTIT |
| RA_D20 | RASQSVSSAVA | SASSLYS | QQASYVKKEIT |
| RA_D21 | RASQSVSSAVA | SASSLYS | QQSSYVHKLIT |
| RA_D23 | RASQSVSSAVA | SASSLYS | QQASYVRREIT |
| RA_D24 | RASQSVSSAVA | SASSLYS | QQASYVHRLIT |

In an embodiment, the binding partner comprises the CDR-H1, CDR-H2, and CDR-H3 of a heavy chain variable region amino acid sequence and/or the CDR-L1, CDR-L2, and CDR-L3 of a light chain variable region of a binding partner selected from the group consisting of RA_D11, RA_D01-RA_D04, RA_D06-RA_D09, RA_D12-RA_D14, RA_D16, RA_D18-RA_D21, RA_D23, and RA_D24, or a variant thereof comprising 1-5 amino acid changes in one or more of the CDR amino acid sequences.

In an embodiment, the binding partner comprises the CDR-H1, CDR-H2, CDR-H3, CDR-L1, CDR-L2, and/or CDR-L3 amino acid sequences of a binding partner selected from the group consisting of RA_D11, RA_D01-RA_D04, RA_D06-RA_D09, RA_D12-RA_D14, RA_D16, RA_D18-RA_D21, RA_D23, and RA_D24 (Tables G and H), or a variant thereof comprising 1-5 amino acid changes in one or more of the CDR amino acid sequences.

In an embodiment, the binding partner can comprise a VH and/or a VL amino acid sequence having at least about 40%, 50%, 55%, 60%, 65%, 70%, 75%, 80%, 85%, 90%, 95%, or 100% sequence identity to the following VH and VL sequences described herein.

In an embodiment, the binding partner can comprise a VH and/or a VL amino acid sequence having at least about 40%, 50%, 55%, 60%, 65%, 70%, 75%, 80%, 85%, 90%, 95%, or 100% sequence identity to a VH and/or a VL of RA_D11, RA_D01-RA_D04, RA_D06-RA_D09, RA_D12-RA_D14, RA_D16, RA_D18-RA_D21, RA_D23, and RA_D24 (Tables G and H).

A non-limiting example of a reference binding partner that binds to a conjugate formed by the covalent reaction of AMG-510 with a KRAS^{G12C} peptide and presented by an HLA molecule is referred to herein as RA_D11.

The amino acid sequence of the light chain variable region (VL) and heavy chain variable region (VH) of RA_D11 are (CDRs are shown in bold; underlining corresponds to residues shown in Table E and F which are presented in the Figures.):

V_{L}: DIQMTQSPSSLSASVGDRVTITCRASQSVSSAVAWYQQKPGKAPKL
LIYSASSLYSGVPSRFSGSRSGTDFTLTISSLQPEDFATYYCQQASYVRK
TITFGQGTKVEIKRTV
V_{H}: EVQLVESGGGLVQPGGSLRLSCAASGFTFSDYSIHWVRQAPGKGLE
WVASISSSSGSTSYADSVKGRFTISADTSKNTAYLQMNSLRAEDTAVYYC
ARGGWIAAMDYWGQGTLVTVSS

A non-limiting example of a reference binding partner that binds to KRAS(G12C)-AMG-510 presented by an HLA molecule is referred to herein as AMRA3-7D. The amino acid sequence of the light chain variable region (VL) and heavy chain variable region (VH) of AMRA3-7D are:

V_{L}:
DIQMTQSPSSLSASVGDRVTITCRASQSVSSAVAWYQQKPGKAPKLLIY
SASSLYSGVPSRFSGSRSGTDFTLTISSLQPEDFATYYCQQISYVKKLI
TFGQGTKVEIKRTV
V_{H}:
EVQLVESGGGLVQPGGSLRLSCAASGFTFSDYSIHWVRQAPGKGLEWVA
SISSSSGSTSYADSVKGRFTISADTSKNTAYLQMNSLRAEDTAVYYCAR
GGWYPAMDYWGQGTLVTVSS

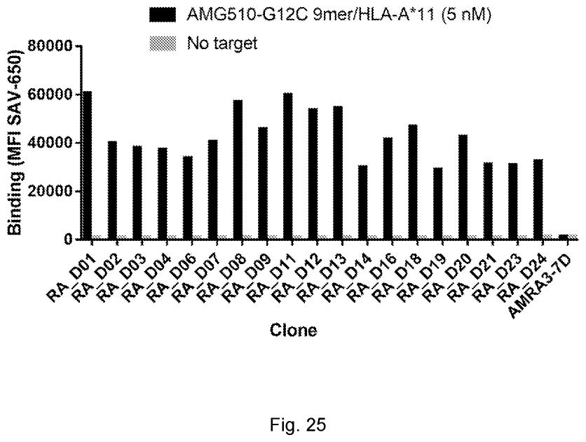

Data that include a comparison of AMRA3-7D and RA-D11 (and other binding partners) binding to an AMG510-G12C 9mer in an HLA-A*11 context is shown in FIG. 25.

In another aspect, the present disclosure provides binding partners (or antigen-binding domains) that have improved binding affinities to the drug-peptide/MHC complex. The peptide can be a KRAS peptide. The drug can be sotorasib (e.g., AMG-510). The drug-peptide conjugate can be a KRAS(G12C) peptide conjugated to sotorasib. The binding partners can bind to KRAS(G12C)-sotorasib presented by an MHC molecule. In some aspects, the MHC can be HLA-A*02:01, HLA-A*03:01, or HLA-A*11:01. The binder partners described herein can comprise various antigen-binding domains disclosed herein.

For example, in some embodiments, the antigen-binding domain comprises a heavy chain variable region (VH), wherein the VH can comprise a heavy chain complementarity determining region 3 (CDR-H3), the CDR-H3 comprising the amino acid sequence of GSWIHAMDY. The VH can further comprise a CDR-H2 comprising the amino acid sequence of SISSSWGVTSYADSVKG. The VH can further comprise a CDR-H1 comprising the amino acid sequence of FHWYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSWIHAMDY; a CDR-H2 sequence of SISSSWGVTSYADSVKG; a CDR-H1 sequence of FHWYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFHWYSIHWVRQAPGKGLEWVASISSSWG VTSYADSVKGRFTISADTSKNTAYLQMNSLRAEDTAVYYCARGSWIHAMDYWGQG TLVTVSS. The VH can comprise a sequence with at least 80% sequence identity to the sequence: EVOLVESGGGLVQPGGSLRLSCAASGFTFHWYSIHWVRQAPGKGLEWVASISSSWG VTSYADSVKGRFTISADTSKNTAYLQMNSLRAEDTAVYYCARGSWIHAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GHWIAAMDY. The VH can further comprise a CDR-H2 comprising the amino acid sequence of SIASSSGSTGYADSVKG. The VH can further comprise a CDR-H1 comprising the amino acid sequence of FSWYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GHWIAAMDY; a CDR-H2 sequence of SIASSSGSTGYADSVKG; a CDR-H1 sequence of FSWYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSWYSIHWVRQAPGKGLEWVASIASSSGS TGYADSVKGRFTISADTSKNTAYLQMNSLRAEDTAVYYCARGHWIAAMDYWGQG TLVTVSS. The VH can comprise a sequence with at least 80% sequence identity to the sequence: EVOLVESGGGLVQPGGSLRLSCAASGFTFSWYSIHWVRQAPGKGLEWVASIASSSGS TGYADSVKGRFTISADTSKNTAYLQMNSLRAEDTAVYYCARGHWIAAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGVIHAMDY. The VH can further comprise a CDR-H2 comprising the amino acid sequence of SILSRWGVTSYADSVKG. The VH can further comprise a CDR-H1 comprising the amino acid sequence of FSPYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGVIHAMDY; a CDR-H2 sequence of SILSRWGVTSYADSVKG; a CDR-H1 sequence of FSPYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSPYSIHWVRQAPGKGLEWVASILSRWGV TSYADSVKGRFTISADTSKNTAYLQMNSLRAEDTAVYYCARGGVIHAMDYWGQGT LVTVSS. The VH can comprise a sequence with at least 80% sequence identity to the sequence: EVOLVESGGGLVQPGGSLRLSCAASGFTFSPYSIHWVRQAPGKGLEWVASILSRWGV TSYADSVKGRFTISADTSKNTAYLQMNSLRAEDTAVYYCARGGVIHAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GSWIAAMDY. The VH can further comprise a CDR-H2 comprising the amino acid sequence of SISSWHGETGYADSVKG. The VH can further comprise a CDR-H1 comprising the amino acid sequence of FSPYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSWIAAMDY; a CDR-H2 sequence of SISSWHGETGYADSVKG; a CDR-H1 sequence of FSPYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSPYSIHWVRQAPGKGLEWVASISSWHGE TGYADSVKGRFTISADTSKNTAYLQMNSLRAEDTAVYYCARGSWIAAMDYWGQGT LVTVSS. The VH can comprise a sequence with at least 80% sequence identity to the sequence: EVOLVESGGGLVQPGGSLRLSCAASGFTFSPYSIHWVRQAPGKGLEWVASISSWHGE TGYADSVKGRFTISADTSKNTAYLQMNSLRAEDTAVYYCARGSWIAAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGWIAAMDY. The VH can further comprise a CDR-H2 comprising the amino acid sequence of SISSLQGDTGYADSVKG. The VH can further comprise a CDR-H1 comprising the amino acid sequence of FSWYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGWIAAMDY; a CDR-H2 sequence of SISSLQGDTGYADSVKG; a CDR-H1 sequence of FSWYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSWYSIHWVRQAPGKGLEWVASISSLQGD TGYADSVKGRFTISADTSKNTAYLQMNSLRAEDTAVYYCARGGWIAAMDYWGQG TLVTVSS. The VH can comprise a sequence with at least 80% sequence identity to the sequence: EVOLVESGGGLVQPGGSLRLSCAASGFTFSWYSIHWVRQAPGKGLEWVASISSLQGD TGYADSVKGRFTISADTSKNTAYLQMNSLRAEDTAVYYCARGGWIAAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GSWIAAMDY. The VH can further comprise a CDR-H2 comprising the amino acid sequence of SIASWYGDTGYADSVKG. The VH can further comprise a CDR-H1 comprising the amino acid sequence of FHYYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSWIAAMDY; a CDR-H2 sequence of SIASWYGDTGYADSVKG; a CDR-H1 sequence of FHYYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFHYYSIHWVRQAPGKGLEWVASIASWYG DTGYADSVKGRFTISADTSKNTAYLQMNSLRAEDTAVYYCARGSWIAAMDYWGQG TLVTVSS. The VH can comprise a sequence with at least 80% sequence identity to the sequence: EVOLVESGGGLVQPGGSLRLSCAASGFTFHYYSIHWVRQAPGKGLEWVASIASWYG DTGYADSVKGRFTISADTSKNTAYLQMNSLRAEDTAVYYCARGSWIAAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGRIEAMDY. The VH can further comprise a CDR-H2 comprising the amino acid sequence of SISSWYGKTGYADSVKG. The VH can further comprise a CDR-H1 comprising the amino acid sequence of FGYYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGRIEAMDY; a CDR-H2 sequence of SISSWYGKTGYADSVKG; a CDR-H1 sequence of FGYYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFGYYSIHWVRQAPGKGLEWVASISSWYG KTGYADSVKGRFTISADTSKNTAYLQMNSLRAEDTAVYYCARGGRIEAMDYWGQG TLVTVSS. The VH can comprise a sequence with at least 80% sequence identity to the sequence: EVOLVESGGGLVQPGGSLRLSCAASGFTFGYYSIHWVRQAPGKGLEWVASISSWYG KTGYADSVKGRFTISADTSKNTAYLQMNSLRAEDTAVYYCARGGRIEAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GYWIEAMDY. The VH can further comprise a CDR-H2 comprising the amino acid sequence of SIASSYGSTGYADSVKG. The VH can further comprise a CDR-H1 comprising the amino acid sequence of FSKYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GYWIEAMDY; a CDR-H2 sequence of SIASSYGSTGYADSVKG; a CDR-H1 sequence of FSKYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSKYSIHWVRQAPGKGLEWVASIASSYGS TGYADSVKGRFTISADTSKNTAYLQMNSLRAEDTAVYYCARGYWIEAMDYWGQGT LVTVSS. The VH can comprise a sequence with at least 80% sequence identity to the sequence: EVOLVESGGGLVQPGGSLRLSCAASGFTFSKYSIHWVRQAPGKGLEWVASIASSYGS TGYADSVKGRFTISADTSKNTAYLQMNSLRAEDTAVYYCARGYWIEAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GSWIAAMDY. The VH can further comprise a CDR-H2 comprising the amino acid sequence of SIHSSIGTTGYADSVKG. The VH can further comprise a CDR-H1 comprising the amino acid sequence of FGLYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSWIAAMDY; a CDR-H2 sequence of SIHSSIGTTGYADSVKG; a CDR-H1 sequence of FGLYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVQLVESGGGLVQPGGSLRLSCAASGFTFGLYSIHWVRQAPGKGLEWVASIHSSIGT TGYADSVKGRFTISADTSKNTAYLQMNSLRAEDTAVYYCARGSWIAAMDYWGQGT LVTVSS. The VH can comprise a sequence with at least 80% sequence identity to the sequence: EVOLVESGGGLVQPGGSLRLSCAASGFTFGLYSIHWVRQAPGKGLEWVASIHSSIGT TGYADSVKGRFTISADTSKNTAYLQMNSLRAEDTAVYYCARGSWIAAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GSVIHAMDY. The VH can further comprise a CDR-H2 comprising the amino acid sequence of SILSWIGKTSYADSVKG. The VH can further comprise a CDR-H1 comprising the amino acid sequence of FSPYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSVIHAMDY; a CDR-H2 sequence of SILSWIGKTSYADSVKG; a CDR-H1 sequence of FSPYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSPYSIHWVRQAPGKGLEWVASILSWIGK TSYADSVKGRFTISADTSKNTAYLQMNSLRAEDTAVYYCARGSVIHAMDYWGQGT LVTVSS. The VH can comprise a sequence with at least 80% sequence identity to the sequence: EVOLVESGGGLVQPGGSLRLSCAASGFTFSPYSIHWVRQAPGKGLEWVASILSWIGK TSYADSVKGRFTISADTSKNTAYLQMNSLRAEDTAVYYCARGSVIHAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGWIAAMDY. The VH can further comprise a CDR-H2 comprising the amino acid sequence of SIASRWGHTGYADSVKG. The VH can further comprise a CDR-H1 comprising the amino acid sequence of FSPYHIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGWIAAMDY; a CDR-H2 sequence of SIASRWGHTGYADSVKG; a CDR-H1 sequence of FSPYHIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSPYHIHWVRQAPGKGLEWVASIASRWG HTGYADSVKGRFTISADTSKNTAYLQMNSLRAEDTAVYYCARGGWIAAMDYWGQ GTLVTVSS. The VH can comprise a sequence with at least 80% sequence identity to the sequence: EVQLVESGGGLVQPGGSLRLSCAASGFTFSPYHIHWVRQAPGKGLEWVASIASRWG HTGYADSVKGRFTISADTSKNTAYLQMNSLRAEDTAVYYCARGGWIAAMDYWGQ GTLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GSWIAAMDY. The VH can further comprise a CDR-H2 comprising the amino acid sequence of SIASLQGITGYADSVKG. The VH can further comprise a CDR-H1 comprising the amino acid sequence of FHEYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GSWIAAMDY; a CDR-H2 sequence of SIASLQGITGYADSVKG; a CDR-H1 sequence of FHEYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFHEYSIHWVRQAPGKGLEWVASIASLQGI TGYADSVKGRFTISADTSKNTAYLQMNSLRAEDTAVYYCARGSWIAAMDYWGQGT LVTVSS. The VH can comprise a sequence with at least 80% sequence identity to the sequence: EVOLVESGGGLVQPGGSLRLSCAASGFTFHEYSIHWVRQAPGKGLEWVASIASLQGI TGYADSVKGRFTISADTSKNTAYLQMNSLRAEDTAVYYCARGSWIAAMDYWGQGT LVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGVIHAMDY. The VH can further comprise a CDR-H2 comprising the amino acid sequence of SILSRWGVTSYADSVKG. The VH can further comprise a CDR-H1 comprising the amino acid sequence of FSDYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGVIHAMDY; a CDR-H2 sequence of SILSRWGVTSYADSVKG; a CDR-H1 sequence of FSDYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSDYSIHWVRQAPGKGLEWVASILSRWG VTSYADSVKGRFTISADTSKNTAYLQMNSLRAEDTAVYYCARGGVIHAMDYWGQG TLVTVSS. The VH can comprise a sequence with at least 80% sequence identity to the sequence: EVOLVESGGGLVQPGGSLRLSCAASGFTFSDYSIHWVRQAPGKGLEWVASILSRWG VTSYADSVKGRFTISADTSKNTAYLQMNSLRAEDTAVYYCARGGVIHAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

In some embodiments, the antigen-binding domain comprises a heavy chain variable region (VH), wherein the VH can comprise a heavy chain complementarity determining region 3 (CDR-H3), the CDR-H3 comprising the amino acid sequence of GGVIHAMDY. The VH can further comprise a CDR-H2 comprising the amino acid sequence of SISSRWGVTSYADSVKG. The VH can further comprise a CDR-H1 comprising the amino acid sequence of FSDYSIH. In some embodiments, the antigen-binding domain can further comprise a light chain variable region (VL), wherein the VL can comprise a CDR-L3 comprising the amino acid sequence of QQASYVRKTIT. The VL can further comprise a CDR-L2 comprising the amino acid sequence of SASSLYS. The VL can further comprise a CDR-L1 comprising the amino acid sequence of RASQSVSSAVA. The antigen binding domain can comprise: a CDR-H3 sequence of GGVIHAMDY; a CDR-H2 sequence of SISSRWGVTSYADSVKG; a CDR-H1 sequence of FSDYSIH; a CDR-L3 sequence of QQASYVRKTIT; a CDR-L2 sequence of SASSLYS; and a CDR-L1 sequence of RASQSVSSAVA. The VH can comprise a sequence with at least 50%, 55%, 60%, 65%, 70%, 75%, 80%, 85%, 90%, 95%, 96%, 97%, 98%, 99%, or 100% sequence identity to the sequence: EVOLVESGGGLVQPGGSLRLSCAASGFTFSDYSIHWVRQAPGKGLEWVASISSRWG VTSYADSVKGRFTISADTSKNTAYLQMNSLRAEDTAVYYCARGGVIHAMDYWGQG TLVTVSS. The VH can comprise a sequence with at least 80% sequence identity to the sequence: EVOLVESGGGLVQPGGSLRLSCAASGFTFSDYSIHWVRQAPGKGLEWVASISSRWG VTSYADSVKGRFTISADTSKNTAYLQMNSLRAEDTAVYYCARGGVIHAMDYWGQG TLVTVSS. The VL can comprise a sequence with at least 50%, 55%, 60%, 65%, 70%, 75%, 80%, 85%, 90%, 95%, 96%, 97%, 98%, 99%, or 100% sequence identity to the sequence: DIQMTQSPSSLSASVGDRVTITCRASQSVSSAVAWYQQKPGKAPKLLIYSASSLYSGV PSRFSGSRSGTDFTLTISSLQPEDFATYYCQQASYVRKTITFGQGTKVEIKRTV. The VL can comprise a sequence with at least 80% sequence identity to the sequence:

DIQMTQSPSSLSASVGDRVTITCRASQSVSSAVAWYQQKPGKAPKLLIY
SASSLYSGVPSRFSGSRSGTDFTLTISSLQPEDFATYYCQQASYVRKTI
TFGQGTKVEIKRTV.

The CDRs for any binding partners or antigen-binding domains described herein can be designated by Kabat numbering scheme. In some cases, the light chain (LC) CDRs can be designated by Kabat numbering scheme. In some cases, the LC CDRs can be designated by Kabat numbering scheme with modifications. In some cases, the heavy chain (HC) CDRs can be designated by Kabat numbering scheme. In some cases, the HC CDRs can be designated by Kabat numbering scheme with modifications. For example, the CDRs may comprise one or more extra amino acids than the CDRs designated by Kabat numbering scheme.

In an aspect, provided herein is a binding partner that specifically binds to a conjugate formed by the covalent reaction of osimertinib with an EGFR peptide presented by an HLA, wherein the binding partner comprises a heavy chain variable region (VH) and a light chain variable region (VL), wherein
(a) the VH comprises:
   (i) a CDR-H1 comprising the amino acid sequence of SSYIH, or a variant thereof comprising 1-5 amino acid changes;
   (ii) a CDR-H2 comprising the amino acid sequence of YISPSYGSTSYADSVKG, or a variant thereof comprising 1-5 amino acid changes; and/or
   (iii) a CDR-H3 comprising the amino acid sequence of EX₁X₂X₃MX₄X₅DY, wherein X₁ is Y, L, S, or E, X₂ is V, T, or I, X₃ is T or I, X₄ is A, T, or S, and X₅ is L, A, I, K, P, or T; and/or
(b) the VL comprises:
   (i) a CDR-L1 comprising the amino acid sequence of RASQSVSSAVA, or a variant thereof comprising 1-5 amino acid changes;
   (ii) a CDR-L2 comprising the amino acid sequence of SASSLYS, or a variant thereof comprising 1-5 amino acid changes; and/or
   (iii) a CDR-L3 comprising the amino acid sequence of QQYX₁X₂WPX₃T, wherein X₁ is S or A, or S; X₂ is Y, H, A, D, E, K, S, or G; and X₃ is I or E.

The CDR sequences of exemplary antibodies that bind a conjugate formed by the covalent reaction of osimertinib with an EGFR peptide presented on an HLA are provided in Table I and Table J below.

TABLE I
Heavy chain CDR sequences of exemplary antibodies that
bind a conjugate formed by the covalent reaction of
osimertinib with an EGFR peptide presented on an HLA.

| Antibody | CDRH1 | CDRH2 | CDRH3 |
|---|---|---|---|
| OEA2-5 | SSYIH | YISPSYGSTSYADSVKG | EYVTMALDY |
| EO_Q01 | SSYIH | YISPSYGSTSYADSVKG | ELVTMTADY |
| EO_Q02 | SSYIH | YISPSYGSTSYADSVKG | EYTTMSIDY |
| EO_Q03 | SSYIH | YISPSYGSTSYADSVKG | ESVTMSADY |
| EO_Q04 | SSYIH | YISPSYGSTSYADSVKG | ESVTMTKDY |
| EO_Q05 | SSYIH | YISPSYGSTSYADSVKG | EEVTMSIDY |
| EO_Q06 | SSYIH | YISPSYGSTSYADSVKG | ESITMTKDY |
| EO_Q07 | SSYIH | YISPSYGSTSYADSVKG | ELTTMSIDY |
| EO_Q08 | SSYIH | YISPSYGSTSYADSVKG | ELTEMTTDY |
| EO_Q09 | SSYIH | YISPSYGSTSYADSVKG | EEVTMTADY |
| EO_Q10 | SSYIH | YISPSYGSTSYADSVKG | ELIEMTPDY |
| EO_Q11 | SSYIH | YISPSYGSTSYADSVKG | ELVTMTIDY |
| EO_Q12 | SSYIH | YISPSYGSTSYADSVKG | ELTTMSIDY |
| EO_Q13 | SSYIH | YISPSYGSTSYADSVKG | ELTTMTADY |
| EO_Q14 | SSYIH | YISPSYGSTSYADSVKG | ESITMSPDY |
| EO_Q15 | SSYIH | YISPSYGSTSYADSVKG | ELIEMTTDY |
| EO_Q16 | SSYIH | YISPSYGSTSYADSVKG | EYVTMSIDY |
| EO_Q17 | SSYIH | YISPSYGSTSYADSVKG | ELVEMTPDY |
| EO_Q18 | SSYIH | YISPSYGSTSYADSVKG | EYVTMALDY |
| EO_Q20 | SSYIH | YISPSYGSTSYADSVKG | EEVTMTPDY |
| EO_Q22 | SSYIH | YISPSYGSTSYADSVKG | EYVTMALDY |
| EO_Q23 | SSYIH | YISPSYGSTSYADSVKG | EYVTMALDY |
| EO_Q24 | SSYIH | YISPSYGSTSYADSVKG | EYVTMALDY |

TABLE J
Light chain CDR sequences of exemplary antibodies that
bind a conjugate formed by the covalent reaction of
osimertinib with an EGFR peptide presented on an HLA.

| Antibody | CDR-L1 | CDR-L2 | CDR-L3 |
|---|---|---|---|
| OEA2-5 | RASQSVSSAVA | SASSLYS | QQYSYWPIT |
| EO_Q01 | RASQSVSSAVA | SASSLYS | QQYSHWPIT |
| EO_Q02 | RASQSVSSAVA | SASSLYS | QQYSAWPET |
| EO_Q03 | RASQSVSSAVA | SASSLYS | QQYSAWPIT |
| EO_Q04 | RASQSVSSAVA | SASSLYS | QQYSDWPIT |
| EO_Q05 | RASQSVSSAVA | SASSLYS | QQYAEWPIT |
| EO_Q06 | RASQSVSSAVA | SASSLYS | QQYSHWPIT |
| EO_Q07 | RASQSVSSAVA | SASSLYS | QQYAKWPIT |
| EO_Q08 | RASQSVSSAVA | SASSLYS | QQYSSWPIT |
| EO_Q09 | RASQSVSSAVA | SASSLYS | QQYSHWPIT |
| EO_Q10 | RASQSVSSAVA | SASSLYS | QQYSGWPIT |
| EO_Q11 | RASQSVSSAVA | SASSLYS | QQYSGWPIT |
| EO_Q12 | RASQSVSSAVA | SASSLYS | QQYASWPIT |
| EO_Q13 | RASQSVSSAVA | SASSLYS | QQYSHWPIT |
| EO_Q14 | RASQSVSSAVA | SASSLYS | QQYSSWPIT |
| EO_Q15 | RASQSVSSAVA | SASSLYS | QQYSHWPIT |
| EO Q16 | RASQSVSSAVA | SASSLYS | QQYSDWPIT |
| EO_Q17 | RASQSVSSAVA | SASSLYS | QQYSSWPIT |
| EO_Q18 | RASQSVSSAVA | SASSLYS | QQYSAWPET |
| EO Q20 | RASQSVSSAVA | SASSLYS | QQYSDWPIT |
| EO_Q22 | RASQSVSSAVA | SASSLYS | QQYSHWPIT |
| EO_Q23 | RASQSVSSAVA | SASSLYS | QQYSHWPET |
| EO_Q24 | RASQSVSSAVA | SASSLYS | QQYSEWPET |

In an embodiment, the binding partner comprises the CDR-H1, CDR-H2, and CDR-H3 of a heavy chain variable region amino acid sequence and/or the CDR-L1, CDR-L2, and CDR-L3 of a light chain variable region of a binding partner selected from the group consisting of OEA2-5, EO_Q01-EO_Q18, and EO_Q20-EO_Q24, or a variant thereof comprising 1-5 amino acid changes in one or more of the CDR amino acid sequences.

In an embodiment, the binding partner comprises the CDR-H1, CDR-H2, CDR-H3, CDR-L1, CDR-L2, and/or CDR-L3 amino acid sequences of a binding partner selected from the group consisting of OEA2-5, EO_Q01-EO_Q18, and EO_Q20-EO_Q24 (Tables I and J), or a variant thereof comprising 1-5 amino acid changes in one or more of the CDR amino acid sequences.

In an embodiment, the binding partner can comprise a VH and/or a VL amino acid sequence having at least about 40%, 50%, 55%, 60%, 65%, 70%, 75%, 80%, 85%, 90%, 95%, or 100% sequence identity a VH and/or a VL amino acid sequence of an antibody disclosed in Tables I and J.

A non-limiting example of a binding partner that binds to an Epidermal Growth Factor receptor (EGFR)-osimertinib conjugate is referred to herein as OEA2-5. The disclosure includes all derivatives of OEA2-5 that are described herein, including the alternative residues described below by way of deep mutational analysis, and in the form of an scDb, for which representative amino acid sequences are provided.

The amino acid sequences of the light chain variable region (VL) and heavy chain variable region (VH) of OEA2-5 are:

V_{L}:
DIQMTQSPSSLSASVGDRVTITCRASQSVSSAVAWYQQKPGKAPKL
LIYSASSLYSGVPSRFSGSRSGTDFTLTISSLQPEDFATYYCQQYS
YWPITFGQGTKVEIKRTV
V_{H}:
EVQLVESGGGLVQPGGSLRLSCAASGFTISSSYIHWVRQAPGKGLE
WVAYISPSYGSTSYADSVKGRFTISADTSKNTAYLQMNSLRAEDTA
VYYCAREYVTMALDYWGQGTLVTVSS

In embodiments, the binding partner binds to a protein in its native form, with the exception that the drug or other molecule is covalently attached to it. “Native form” means the intact protein that retains its biological function before covalent attachment of the drug or other molecule. In embodiments, the native form or the protein is its form before being fragmented such as by intracellular processing. In embodiments, the binding proteins therefore bind to full length polypeptides that are covalently attached to the drug or other molecule and wherein the covalently bound drug or other molecule at least in part permits the preferential binding of the binding partners. In general, a polypeptide, which is used interchangeably herein with the term “protein,” comprises more than 50 contiguous amino acids. In embodiments, a binding partner binds with specificity to an intact protein that is covalently attached to a drug or other molecule. In other embodiments, the binding partners bind with specificity to a peptide comprising the covalently bound molecule. In embodiments, the binding partner binds with specificity to a peptide having a specific amino acid sequence and is covalently conjugated to another molecule, such as a drug. In embodiments, the binding partner binds preferentially to a peptide covalently bound to a molecule such as a drug, where the sequence of the peptide is not relevant. This preferential binding is relative to binding to the same peptide that is not conjugated to the drug. In embodiments, the binding partner binds preferentially to a peptide comprising a KRAS (G12) mutation, or to a variant thereof, wherein the variant is at least 50% similar to the KRAS (G12)-containing peptide. This preferential binding is relative to binding to a KRAS (G12)-containing peptide, or the variant thereof, respectively, that is not covalently conjugated to the drug or other molecule.

In embodiments, the described binding partners bind with specificity to peptide conjugates that are of suitable length to be presented in a major histocompatibility complex (MHC), referred to as human leukocyte antigen (HLA) in humans, or to MHC or its equivalent complex in non-human animals, including but not limited to non-human mammals.

In general, the peptide conjugate comprises fewer than 50 contiguous amino acids. In embodiments, the peptide conjugates which comprise the described epitopes may therefore be from 2-49 amino acids in length. In embodiments, the peptide to which the drug or other molecule is covalently attached, and which attached drug and one or more residues of the peptide may be comprised by the epitope, comprises from 4-12 contiguous amino acids, which may or may not be derived from a longer protein during the processing of a protein, such as an antigen processed for presentation by an MHC molecule. In embodiments, the drug is conjugated to a peptide that comprises, or consists of 7-30 amino acids. In embodiments, the drug or other molecule is conjugated to a peptide that comprises, or consists of, 7, 8, 9, 10, 11, 12, 13, 14, or 15 amino acids, and which may be presented in an MHC Class I context. In embodiments, the drug or other molecule is conjugated to a peptide that is 9-30 amino acids, inclusive, and including all numbers and ranges of numbers there between, and which may be presented in an MHC Class II context. In embodiments, the drug or other molecule is conjugated to a peptide comprises at least 7 amino acids. Non-classical MHC class I molecules function to mediate inhibitory or activating stimuli in natural killer (NK) cells. Non-classical MHC class I molecules can be expressed by immune and tumor cells. For example, expression of non-classical MHC complexes on malignant cells hampers cytotoxic activity of effector cells in the immune system. Overexpression of non-classical MHC class I molecules, including but not limited to HLA-E, HLA-F, and HLA-G, can be found in cancer cells. In some embodiments, the drug or other molecule is conjugated to a peptide that is 9-30 amino acids, inclusive, and including all numbers and ranges of numbers there between, and which may be presented in a non-classical MHC Class I context. In some embodiments, the peptide conjugate forms a complex with a non-classical MHC class I molecule. In some embodiments, the non-classical MHC class I molecule is selected from the group consisting of HLA-E, HLA-F, and HLA-G. In some embodiments, the non-classical MHC class I molecule is selected from HLA-E, HLA-F, HLA-G, or some combination thereof.

In embodiments, a binding partner binds with specificity to a peptide conjugate that is covalently conjugated to a drug or other molecule independent of MHC presentation. In embodiments, non-limiting examples of which are described below in Example 3, the binding partner binds with specificity to the peptide conjugate only when the peptide conjugate is presented by an MHC molecule. In embodiments, the binding partner can bind with specificity to a peptide conjugate in both an MHC-independent and an MHC-presented context. In embodiments, the MHC-peptide conjugate complex comprises an antigen to which a described binding partner binds with specificity.

In embodiments, a described binding partner exhibits at least one improved property relative to the same property of a reference binding partner. In embodiments, a reference binding partner is AMRA3-7D. In one embodiment, a described binding partner exhibits a greater affinity for its target relative to a reference binding partner.

In embodiments, the binding partners accordingly can bind to cells via any MHC that can present peptide conjugates. In embodiments, the HLA is expressed by cells that are capable of Class I, Class II, or Class III MHC presentation. In embodiments, the binding partners can bind to cells that express Class I MHC that presents the peptide conjugate. Those skilled in the art will recognize that Class I MHC includes, among other components, a polymorphic a chain and B2 microglobulin, wherein the peptide conjugate binds to the polymorphic chain.

In embodiments, the cells are antigen presenting cells (APCs). In embodiments, the cells are so-called professional antigen presenting cells, and thus may include but are not limited to macrophages and dendritic cells, which display Class II MHC. Those skilled in the art will recognize that Class II MHC includes, among other components, MHC polymorphic a and B chains, and the displayed peptide conjugate binds to both chains. In other embodiments, Class II MHC may be displayed with the peptide conjugate by other cell types, such as cancer/tumor cells, and thus the disclosure provides for direct recognition of such cells using the described binding partners, without requirement for a professional APC.

In embodiments, the peptide conjugate is displayed by a non-classical MHC complex, which may include CD1d, MR1, MHC-E, -F, -G and/or other emerging family members that will be recognized by those skilled in the art.

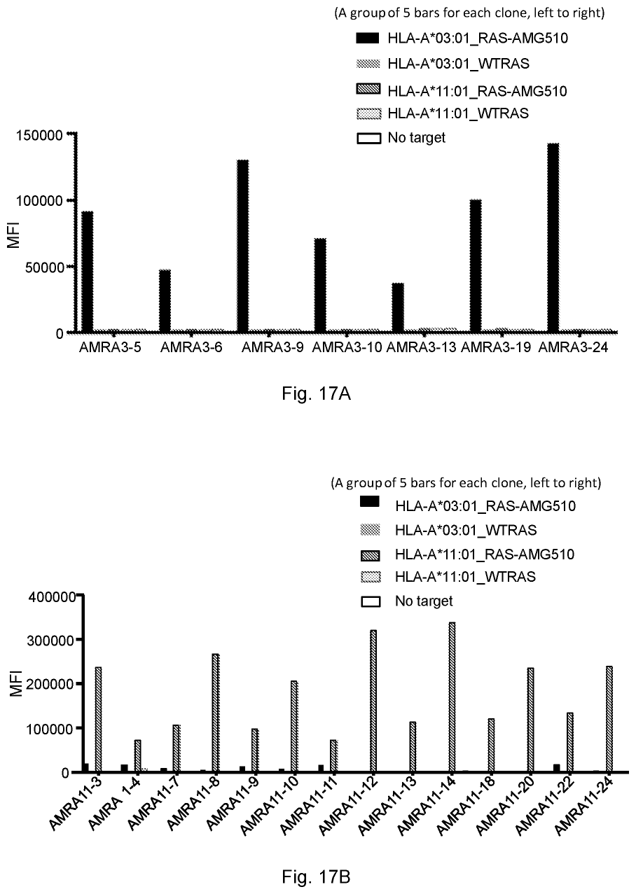

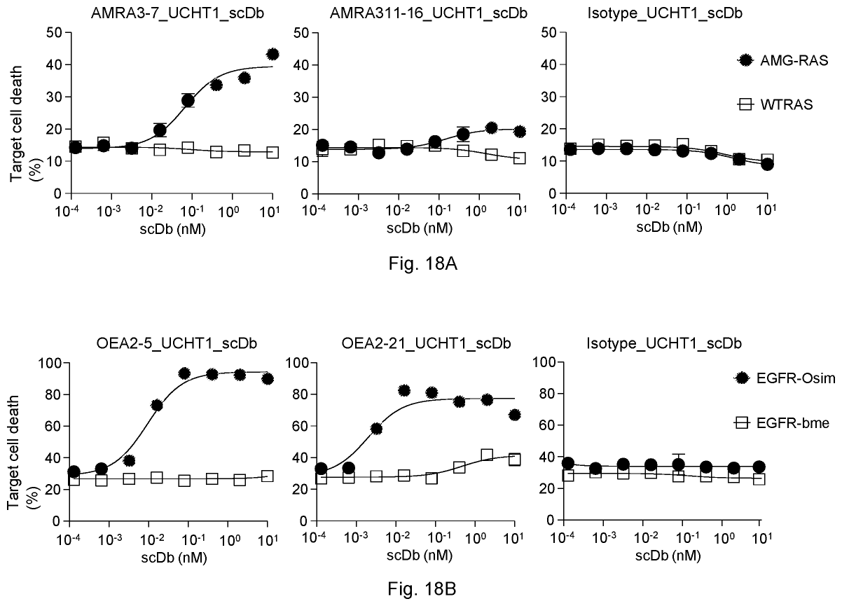

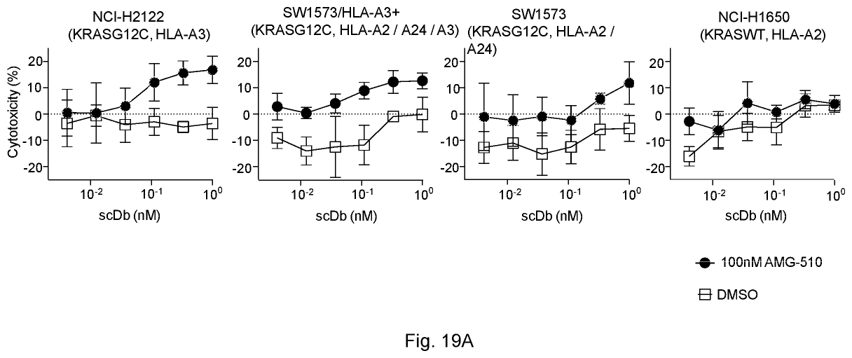

The disclosure includes binding partners that bind with specificity to a peptide conjugate displayed only by a specific MHC type, and thus provides binding partners that discriminate between MHC types. Representative examples of such binding partners are described herein at least by way of FIG. 17.

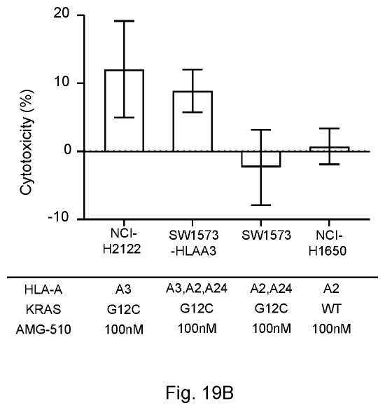

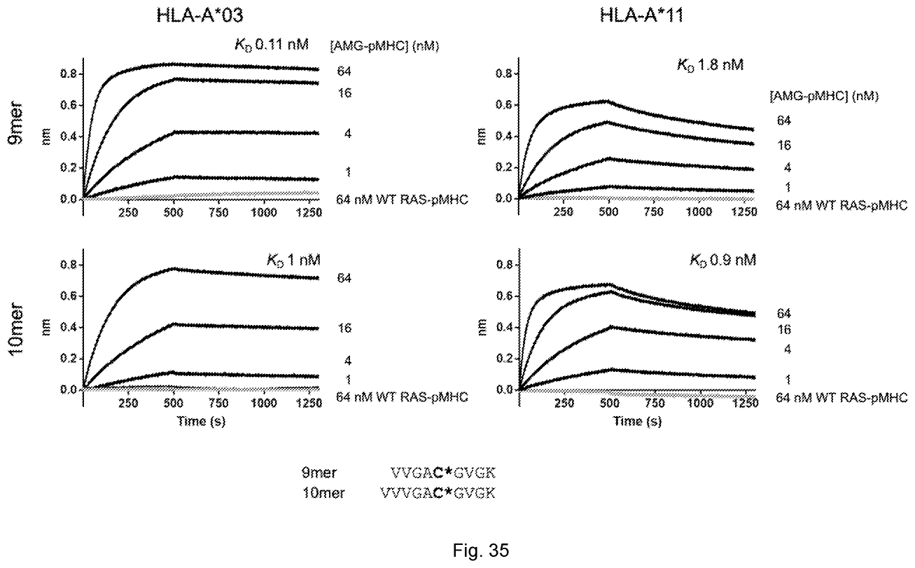

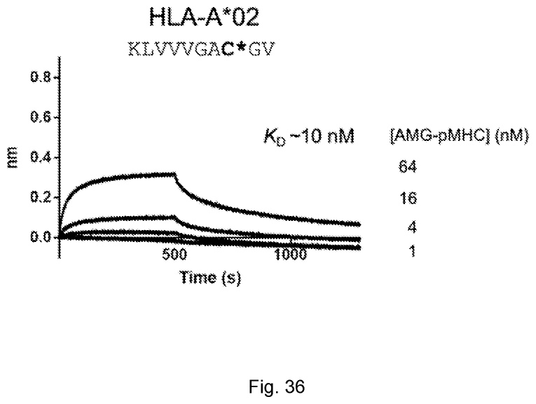

In embodiments, a binding partner of this disclosure can bind with specificity to a peptide conjugate comprising a covalently conjugated drug or other molecule that is displayed by more than one specific MHC type. In embodiments, a single binding partner of the disclosure is suitable for use with a diversity of HLA types. Furthermore, a single binding partner can bind with specificity multiple combinations of (a) peptide conjugate(s) comprising a covalently conjugated drug or other molecule and (b) HLA types, such as demonstrated by way of FIGS. 35 and 36. In particular, FIGS. 35 and 36 demonstrate that that a single antibody can bind to different peptides conjugated to AMG-510 that are presented by different HLAs having distinct preferences for peptide binding. For example, compare the HLA-A*03/11 peptide, VVVGAC*GVGK with KLVVVGAC*GV, which shows that the antibody RA_D11 still selectively binds to the hapten-peptide-HLA complex relative to binding to the free drug. In embodiments, a binding partner of this disclosure can bind with specificity to a peptide conjugate comprising a covalently conjugated drug only in a specific MHC context. In embodiments, the peptide conjugate is displayed by an MHC class I type selected from HLA-A, -B, -C, and combinations thereof. In certain aspects, the peptide conjugate is displayed in the context of any MHC class I that is A*02/B*35/C*04. In embodiments, the peptide conjugate is displayed by any of MHC of class II that is DR*01/DR*04/DR*07/DP*04. In embodiments, the HLA comprises A*01:01, A*02:01, A*03:01, A*11:01, A*24:02, A*26:01, B*07:02, B*08:01, B*27:05, B*39:01, B*40:01, B*58:01, or B*15:01. Specific examples of antibodies include antibodies that bind to KRAS(G12C)-AMG510 conjugate presented on HLA-A*02:01, HLA-A*03:01 and HLA-A*11:01, BTK-Ibrutinib conjugate presented on HLA-A*01:01, and EGFR-osimertinib conjugate presented on HLA-A*02:01. In non-limiting embodiments, the disclosure provides scDbs that are specific for a particular drug that is covalently bound to a described peptide that is present on a specific HLA, or the same drug that is covalently bound to a described peptide that is present on two different HLAs. Representative scDbs are described in Example 4. Data obtained using the scDbs are presented via FIGS. 18 and 19. Data obtained using CrossMab formats are provided in Example 5 and its accompanying figures.

In embodiments, the disclosure comprises selecting an individual based on the HLA type of the individual and selecting an antibody described herein for treating that individual. In embodiments, the binding partner is selected based at least on part on the degree of the HLA restriction exhibited by the selected binding partner, relative to the HLA type of the individual.

In certain embodiments, such as KRAS(G12C) binding partners, representative examples are provided below and bind to two different peptides derived from KRAS(G12C) conjugated to a drug (AMG-510, also referred to as sotorasib).

In embodiments, the peptide conjugate is displayed by cells that participate in, or can be the targets of, cell-mediated immune responses. In embodiments the peptide conjugate that is displayed in any suitable MHC context is presented by a cell that is recognized by a leukocyte, including but not necessarily limited to a T cell or a natural killer (NK) cell. In embodiments, the T cell is a CD4+ T cell, a CD8+ T cell, a double positive CD4+/CD8+ T cell, a CD4+/CD8+ double negative T cell, or a γδ T cell. Thus, and as described further below, the disclosure provides binding partners that are configured to interact with both the presented peptide conjugate and cells that participate in cell-mediated immune responses. In embodiments, certain described binding partners are capable of binding to a complex of 1) a specific MHC and 2) a specific peptide conjugate. In embodiments, certain described binding partners are capable of being bound to a specific peptide conjugate presented by at least two different MHCs.

In embodiments, any binding partner of this disclosure comprises at least one chain that comprises a complementary determining region (CDR) that is CDR1, CDR2, or CDR3 from any heavy or light chain amino acid sequence described herein. In certain examples in the present specification, the CDRs are shown in bold font. The amino acid sequences of the CDR sequences are separately encompassed by this disclosure by way of their positions in the described heavy and light chain amino acid sequences. The disclosure includes binding partners that comprise a described heavy chain CDR1, CDR2, and CDR3. The disclosure also includes binding partners that comprise a described light chain CDR1, CDR2, and CDR3. The disclosure also includes binding partners that comprise a described heavy chain CDR1, CDR2, and CDR3 and a described light chain CDR1, CDR2, and CDR3. For amino acid sequences of this disclosure that include amino acids that comprise purification or protein production tags, such as HIS tags and/or AVI-tags, the disclosure includes the proviso that the sequences of the described tags may be excluded from the amino acid sequences. Amino acids between the described tags may also be excluded.

Binding partners of this disclosure can be provided as intact immunoglobulins or as fragments of immunoglobulins, including but not necessarily limited to antigen-binding (Fab) fragments, Fab′ fragments, (Fab′)2 fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), isolated complementary determining regions (CDRs), single-chain variable fragment (scFv), and other antibody fragments that retain antigen binding function. In embodiments, one or more binding partners are provided as a component of a bispecific T-cell engager (BiTE), bispecific killer cell engager (BiKE), CrossMab (e.g., a binding partner containing four different chains; immunoglobulin crossover (also known as Fab domain exchange or CrossMab format) technology (see e.g., WO2009/080253; Schaefer et al., Proc. Natl. Acad. Sci. USA, 108:11187-11192 (2011).), or a chimeric antigen receptor (CAR), such as for producing chimeric antigen receptor T cells (e.g., CAR T cells) and CAR natural killer (NK) cells, neutrophil, and macrophages. The disclosure includes binding partners that include the described heavy and light chain variable regions.

The binding partners of this disclosure can be a scFv. In the present disclosure, the VH of the polypeptide can be linked to the VL through a linker. The linker can be a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). In some cases, the linker sequence comprises (G₄S)ₙ, wherein n=2 to 4. In some cases, the linker sequence comprises (G₄S)ₙ, wherein n=1 to 3. In some cases, the linker can comprise (G₄S)ₙ or (S₄G)ₙ where n is any integer from 1 to 10. In some cases, the linker can comprise the glycine-serine-alanine linker G₄SA₃ or the glycine-serine linker (G₄S)₄.

The binding partners of this disclosure can be a bispecific T-cell engager (BiTE). BiTE therapies can be used to connect a subject's endogenous T cells to cancerous cells. A BiTE molecule can comprise two Fv fragments from monoclonal antibodies, joined by a peptide linker. A BiTE molecule can comprise a first antigen binding domain and a second antigen binding domain. The first antigen binding domain can be specific for and bind to a T cell antigen. The second antigen binding domain can bind to a tumor antigen (e.g., a tumor-associated antigen) expressed on the surface of cancerous cells. In some embodiments, the BiTE molecule can specifically bind to a peptide conjugate/MHC complex and a T cell surface antigen. In some embodiments, the T cell surface antigen is CD3 (e.g., CD3 epsilon, CD3 delta, or CD3 gamma), a TCR alpha chain, a TCR beta chain, a TCR gamma chain, a TCR delta chain, ICOS, CD28, CD27, HVEM, LIGHT, CD40, 4-1BB, OX40, DR3, GITR, CD30, TIM1, SLAM, CD2, CD4, CD8, or CD226. In some embodiments, the tumor-associated antigen comprises a peptide conjugate formed by the covalent reaction of a targeted covalent inhibitor with a peptide in a tumor cell. In some embodiments, the tumor-associated antigen comprises a peptide that is a segment of a protein that is associated with cancer, optionally wherein the protein is encoded by a gene that is mutated in a cancer. In some embodiments, the protein is overexpressed in a cancer. In some embodiments, the segment of a protein is overexpressed in cancer. Cancer testis antigens are a class of tumor-associated antigens expressed in human tumors. In some embodiments, the protein is a member of the cancer testis antigen (CTA) family. In some embodiments, the protein associated with cancer comprises an endogenous retrovirus (ERV). In some embodiments, the protein is derived from a retrotransposon family, such as long interspersed elements (LINEs) or short interspersed elements (SINEs). In some embodiments, the tumor-associated antigen is an antigen associated with renal cell carcinoma. In some embodiments, the tumor-associated antigen is an antigen associated with breast cancer. In some embodiments, the tumor-associated antigen is an antigen associated with prostate cancer. In some embodiments, the tumor-associated antigen is an antigen associated with pancreatic cancer. In some embodiments, the tumor-associated antigen is an antigen associated with lung cancer. In some embodiments, the tumor-associated antigen is an antigen associated with liver cancer. In some embodiments, the tumor-associated antigen is an antigen associated with ovarian cancer. In some embodiments, the tumor-associated antigen is an antigen associated with cervical cancer. In some embodiments, the tumor-associated antigen is an antigen associated with colon cancer (or colorectal cancer). In some embodiments, the tumor-associated antigen is an antigen associated with esophageal cancer. In some embodiments, the tumor-associated antigen is an antigen associated with glioma. In some embodiments, the tumor-associated antigen is an antigen associated with glioblastoma or another brain cancer. In some embodiments, the tumor-associated antigen is an antigen associated with stomach cancer. In some embodiments, the tumor-associated antigen is an antigen associated with bladder cancer. In some embodiments, the tumor-associated antigen is an antigen associated with testicular cancer. In some embodiments, the tumor-associated antigen is an antigen associated with head and neck cancer. In some embodiments, the tumor-associated antigen is an antigen associated with melanoma or another skin cancer. In some embodiments, the tumor-associated antigen is an antigen associated with any sarcoma including but not limited to fibrosarcoma, angiosarcoma, osteosarcoma, and rhabdomyosarcoma. In some embodiments, the tumor-associated antigen is an antigen associated with any blood cancer, including all types of leukemia, lymphoma, and myeloma.

The binding partners can comprise a first antigen-binding domain that specifically binds to the peptide-conjugate/MHC complex and a second antigen-binding domain that specifically binds to a T cell surface marker. The binding partners can comprise two polypeptide chains. For example, the polypeptide can comprise a first polypeptide chain comprising the first antigen-binding domain and a second polypeptide chain comprising the second antigen-binding domain. In some cases, the two polypeptide chains can comprise a Fc region. The binding partners of this disclosure can be bispecific or multivalent antibodies or antibody fragments comprising a first antigen-binding domain that specifically binds to the peptide-conjugate/MHC complex and a second antigen-binding domain that specifically binds to a T cell surface marker. In some embodiments, the bispecific or multivalent molecule can comprise a Fc region chosen from heavy chain constant regions of human IgM, IgG1, IgG2, IgG3, IgG4, IgA1, and IgA2. The Fc region can be a modified version of the wildtype Fc region. For example, the Fc region can be a silenced version of the Fc region, including but not limited to, LALA Fc, LALAPG Fc, LALAKA Fc, LAGA Fc, LGGR Fc, or LALE Fc. The Fc region can comprise one or more mutations of a wildtype Fc region. The Fc region can be linked to one or more tumor targeting moieties, a T cell engager, or a cytokine molecule. In some embodiments, the interface of a first and second Fc region can be altered to increase or decrease dimerization. The dimerization of the Fc region can be enhanced by providing an Fc interface of a first and a second Fc region with a paired-cavity protuberance, e.g. knob-in-hole. Knob-in-hole as described in U.S. Pat. Nos. 5,731,116, 7,476,724 and Ridgway, J. et al. (1996) Prot. Engineering 9 (7): 617-621, broadly involves: (1) mutating the CH3 domain of one or both antibodies to promote heterodimerization; and (2) combining the mutated antibodies under conditions that promote heterodimerization. “Knobs” or “protuberances” can be created by replacing a small amino acid in a parental antibody with a larger amino acid (e.g. T366Y or T366W). “Holes” or “cavities” are created by replacing a larger residue in a parental antibody with a smaller amino acid (e.g. Y407T, T366S, L368A, and/or Y407V). Exemplary knob-in-hole mutations include S354C or T366W in the “knob” heavy chain and Y349C, T366S, L368A, or Y407V in the “hole” heavy chain. For bispecific antibodies including an Fc domain, introduction of specific mutations into the constant region of the heavy chains to promote the correct heterodimerization of the Fc portion can be used. These techniques include the knob-in-hole approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228). In some cases, the two polypeptide chains can be combined using the knob-in-hole approach described herein. In some cases, the two polypeptide chains cannot be combined using the knob-in-hole approach. In some cases, the first antigen-binding domain and the second antigen-binding domain are linked by a linker. The linker can be a peptide linker that consists of amino acids such as glycine and/or serine residues used alone or in combination. The linker can comprise at least about 1, 2, 5, 10, 15, 20, 25, 30, 35, 40, 45, 50, 55, 60, 65, 70, 75, 80, 85, 90, 95, 100, 150, 200, 250, or more amino acid residues in length. In some cases, the linker can comprise (G₄S)ₙ where n is any integer from 1 to 10. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). In some cases, the linker sequence comprises (G₄S)ₙ, wherein n=2 to 4. In some cases, the linker sequence comprises (G₄S)ₙ, wherein n=1 to 3. In some cases, the linker can comprise (G₄S)ₙ or (S₄G)ₙ where n is any integer from 1 to 10. In some cases, the linker can comprise the glycine-serine-alanine linker G₄SA₃ or a glycine-serine linker (G₄S)₄.

The first or the second polypeptide chain can further be fused to a cytokine or fragment thereof. The cytokine can comprise IL-2, IL-7, IL-15, IL-12, IL-18, or IL-21, or an interferon (IFN). In some cases, the cytokine can be selected from the group consisting of IFNγ, IL-2, IL-4, IL-6, IL-7, IL-10, IL-12, IL-13, IL-15, IL-18, IL-23, IL-24, IL-27, IL-28a, IL-28b, IL-29, KGF, IFN α (e.g., INF α 2b), IFN β, IFN 2, and GM-CSF. In some embodiments, the cytokine is an engineered cytokine. For example, the engineered cytokine can be a variant or mutant of any cytokine described herein. The engineered cytokine can be a mutein, a superkine, or a cytokine with PEGylation. In some embodiments, the cytokine is a single chain cytokine. In some embodiments, the cytokine is a multichain cytokine. In some cases, the two polypeptides can contain two moieties. In some cases, the two polypeptide chains can contain at least three moieties. In some cases, the first moiety can bind to the peptide conjugate/MHC complex, the second moiety can comprise a binding partner for a T cell surface antigen (e.g., T cell engager), and the third moiety can comprise a cytokine or fragment thereof. In some cases, the first polypeptide chain can contain the antigen-binding domain for the peptide conjugate/MHC complex and the second polypeptide chain can contain the antigen-binding domain for a T cell surface antigen. In some cases, the first polypeptide chain can contain the antigen-binding domain for the T cell surface antigen and the second polypeptide chain can contain the antigen-binding domain for the peptide conjugate/MHC complex. In some cases, the first polypeptide chain can contain the antigen-binding domain for the peptide conjugate/MHC complex and a cytokine or fragment thereof and the second polypeptide chain can contain the antigen-binding domain for the T cell surface antigen. In some cases, the first polypeptide chain can contain the antigen-binding domain for the T cell surface antigen and a cytokine or fragment thereof and the second polypeptide chain can contain the antigen-binding domain for the peptide conjugate/MHC complex. In some cases, the first polypeptide chain can contain the antigen-binding domain for the peptide conjugate/MHC complex and the second polypeptide chain can contain the antigen-binding domain for the T cell surface antigen and a cytokine or fragment thereof. In some cases, the first polypeptide chain can contain the antigen-binding domain for the T cell surface marker and the second polypeptide chain can contain the antigen-binding domain for the peptide conjugate/MHC complex and a cytokine or fragment thereof. In various cases, the antigen-binding domain for the peptide conjugate/MHC complex and the antigen-binding domain for the T cell surface antigen can be on a same polypeptide chain, and the cytokine or fragment thereof is fused to a different polypeptide chain. In some cases, the antigen-binding domain for the peptide conjugate/MHC complex and the cytokine or fragment thereof can be on a same polypeptide chain, and the antigen-binding domain for the T cell surface antigen is on a different polypeptide chain. In some cases, the antigen-binding domain for the T cell surface antigen and the cytokine or fragment thereof can be on a same polypeptide chain, and the antigen-binding domain for the peptide conjugate/MHC complex is on a different polypeptide chain.

The polypeptide can be an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BiTE), bispecific killer cell engager (BiKE), CrossMab, a camelid antibody, a tri-specific binding partner, a chimeric antigen receptor (CAR), a Monobody (aka Adnectin), a DARPin, an anticalin, an affibody, a nanobody, or an affimer. In some embodiments, the nanobody is derived from the heavy chain variable domain of antibodies found in members of Camelidae (e.g. camels, llamas, alpacas). In some cases, the polypeptide can be a bispecific antibody. The bispecific antibody can be a bispecific T-cell engager (BiTE). The polypeptide can comprise a first antigen-binding domain and a second antigen-binding domain. The second antigen-binding domain can bind to a T cell surface marker. The T cell surface marker can be CD3 epsilon, CD3 gamma, CD3 delta, a TCR alpha, a TCR beta, a TCR gamma, a TCR delta, ICOS, CD28, CD27, HVEM, LIGHT, CD40, 4-1BB, OX40, DR3, GITR, CD30, TIM1, SLAM, CD2, CD4, CD8, or CD226. In some cases, the antigen-binding domain and the second antigen-binding domain of the polypeptide can be linked by a linker. As described above, the linker can comprise at least about 2, 5, 10, 15, 20, 25, 30, 35, 40, 45, 50, 55, 60, 65, 70, 75, 80, 85, 90, 95, 100, 150, 200, 250, or more amino acid residues in length. The linker can comprise (G₄S)ₙ or (S₄G)ₙ, where n is any integer from 1 to 10.

In various cases, the linker linking the antigen-binding domain and the second antigen-binding domain of the polypeptide can affect the angle between the axis of the MHC and the axis of the polypeptide.

Binding geometry of TCRs to peptide-MHC complexes can be used as a descriptor of binding affinities. Diagonal binding of a TCR to a MHC complex can be quantified using the crossing angle, e.g., the angle between the MHC pocket and the vector between the TCR domains. This angle corresponds to the twist of the receptor over the peptide-MHC complex. The incident angle refers to the angle between the MHC peptide binding groove normal vector and the TCR interdomain axis of rotation. This angle corresponds to the tilt of the TCR over the peptide-MHC complex and is typically measured along the X-Y axis. Binding geometry between MHC complexes and polypeptide disclosed herein can be similarly measured and methods to quantify these binding geometries are known to those in the art (Rudolph et al. How TCRs bind MHCs, peptides, and coreceptors. Annu Rev Immunol. 2006; 24:419-66; Singh et al. Geometrical characterization of T cell receptor binding modes reveals class-specific binding to maximize access to antigen. Proteins. 2020 March; 88 (3):503-513).

The binding geometry between a peptide conjugate/MHC complex and binding partner can be characterized using an incident angle, measured along the X-Y axis, and/or a back angle, measured along the Y-Z axis. In some embodiments, the binding geometry between the polypeptide and peptide conjugate/MHC complex can be given as an incident angle and/or a backangle. In some embodiments, the polypeptide can bind to the peptide conjugate/MHC complex with a back angle from about 10° to 70° (e.g., about 10°, about 20°, about 30°, about 40°, about 50°, about 60°, or about) 70° between the axis of the peptide conjugate/MHC complex and the axis of the polypeptide. In some embodiments, the polypeptide can bind to the peptide conjugate/MHC complex with a back angle from about 60° to 65° between the axis of the peptide conjugate/MHC complex and the axis of the polypeptide. In some embodiments, the polypeptide can bind to the peptide conjugate/MHC complex with a back angle of about 64.7°, 67.2°, or 65.1° between the axis of the peptide conjugate/MHC complex and the axis of the polypeptide.

In some embodiments, the polypeptide can bind to the peptide conjugate/MHC complex with an incident angle from about 10° to 100° (e.g., about 10°, about 20°, about 30°, about 40°, about 50°, about 60°, about 70°, about 80°, about 90, or about) 100° between the axis of the peptide conjugate/MHC complex and the axis of the polypeptide. In some embodiments, the polypeptide can bind to the peptide conjugate/MHC complex with an incident angle from 70° to 80° between the axis of the peptide conjugate/MHC complex and the axis of the polypeptide. In some embodiments, the polypeptide can bind to the peptide conjugate/MHC complex with an incident angle of about 77.2°, 77.3° or 78.4° between the axis of the peptide conjugate/MHC complex and the axis of the polypeptide.

In various cases described herein, the peptide conjugate/MHC complex can be presented on a surface of a cell. The cell can express a low copy number of the peptide conjugate/MHC complex, and wherein the low copy number is at most about 1000, 900, 800, 700, 600, 500, 400, 300, 200, 100, 90, 80, 70, 60, 50, 40, 30, 20, 10, 5, 3, or 1 copy per a single cell. The peptide of the peptide conjugate/MHC complex can be from an intracellular protein. For example, the peptide can be processed from an endogenous or an intracellular protein and presented on the MHC complex.

The binding partners described herein can be a T-cell receptor (TCR) or a functional fragment thereof (e.g., antigen-binding fragment, variable region or extracellular domain of a TCR). The binding partners described herein can be a TCR mimetic binder. The T-cell receptor or the functional fragment thereof can bind specifically to the peptide conjugate/MHC complex described herein. In some cases, the K_{D} of the TCR mimetic binder for the peptide conjugate/MHC complex can be less than about 1 μM, less than about 500 nM, less than about 400 nM, less than about 300 nM, less than about 200 nM, less than about 100 nM, less than about 50 nM, less than about 40 nM, less than about 30 nM, less than about 20 nM, less than about 10 nM, less than about 5 nM, less than about 1 nM, less than about 0.1 nM, less than about 50 pM, less than about 20 pM, less than about 10 pM, less than about 1 pM, less than about 0.1 pM, or less. In some cases, the K_{D} of the TCR for the peptide conjugate/MHC complex can be less than about 1 μM, less than about 500 nM, less than about 400 nM, less than about 300 nM, less than about 200 nM, less than about 100 nM, less than about 50 nM, less than about 40 nM, less than about 30 nM, less than about 20 nM, less than about 10 nM, less than about 5 nM, less than about 1 nM, or less than about 0.1 nM, less than about 50 pM, less than about 20 pM, less than about 10 pM, less than about 1 pM, less than about 0.1 pM, or less. In an aspect, the present disclosure provides a method of identifying a T-cell receptor (TCR) that recognizes the peptide conjugate/MHC complex described herein. In some embodiments, the method of identifying a TCR comprises contacting a plurality of candidate TCRs with the peptide conjugate/MHC complex and identifying at least one TCR that binds to the peptide conjugate/MHC complex. In some embodiments, the method of identifying a TCR that recognizes the peptide conjugate/MHC complex described herein comprises selecting or isolating at least one TCR. Isolation of a TCR can be achieved through analysis of binding affinities to peptide conjugate/MHC complexes. In some embodiments, the peptide-drug conjugate can be a vaccine that enhances isolation of TCRs. In some embodiments, the vaccine can be used to augment G12C-inhibitory therapy. In some embodiments, the vaccine can be an RNA vaccine. For example, the vaccine can be an RNA vaccine encoding a KRAS peptide comprising G12C mutation. In some embodiments, the vaccine can be an RNA vaccine encoding a KRAS peptide comprising G12C mutation that is co-administered with the KRAS peptide comprising G12C mutation. In some embodiments, the vaccine can augment reactivity in patients treated with, for example, G12C-inhibitory therapy or with HapImmune™ antibodies.

In some instances, the plurality of candidate TCRs is a plurality of soluble TCRs. In some instances, the plurality of candidate TCRs is a plurality of TCRs expressed on cell surface of a plurality of cells. In some embodiments, the method of identifying a TCR that recognizes the peptide conjugate/MHC complex described herein comprises isolating or selecting a cell comprising at least one TCR based on an activation marker of the cell. In some embodiments, the activation marker is a T cell marker. In embodiments, the T cell activation marker is CD26, CD27, CD28, CD30, CD154, CD40L, CD134. CD25, CD44, CD69, CD137, or KLRG1. In another aspect, the disclosure provides a T-cell receptor (TCR) comprising at least one identified TCR that recognizes the peptide conjugate/MHC complex described herein. In some embodiments, the TCR is a soluble TCR. In embodiments, the TCR is a bispecific TCR. In some embodiments, the TCR is expressed on a CD4+ T cell. In some embodiments, the TCR is expressed on a CD8+ T cell.

In embodiments, the binding partners are multivalent. In embodiments, a tri-specific binding partner is provided. In embodiments, cells express at least a segment of one or more binding partners in the form of a CAR. In an embodiment, a binding partner of this disclosure may be provided as a complex with a polynucleotide, such as an RNA polynucleotide, to form an aptamer. In embodiments, a multi-valent binding partner includes one binding component, such as a paratope, that confers specificity to a particular target on a desired cell type, such as any cancer cell marker. In embodiments, a tri-specific leukocyte engager is provided. In embodiments, the binding partners may be part of a molecule that is activated only in the presence of a protease or other enzyme present in a tumor microenvironment, such embodiments being pertinent to, for instance, a probody, examples of which are known in the art, for example in doi:10.1126/scitranslmed.3006682, doi:10.1038/s41467-020-16838-w, and doi:10.1038/s41587-019-0135-x, from which the descriptions of probodies, and protease activation, are incorporated herein by reference. In an embodiment, the disclosure provides a universal hapten that can be grafted onto inhibitors.

In embodiments, a CAR of this disclosure comprises scFv that comprises heavy and light chain variable regions as described herein. As is known in the art for previously described CARs, the scFv is present in a contiguous polypeptide that further comprises a CD3zeta chain and a costimulatory domain. In some embodiments, the contiguous polypeptide further comprises a CD3gamma chain or a CD3epsilon chain. In embodiments, the costimulatory domain comprises a 4-1BB costimulatory domain or a CD28 costimulatory domain. A CAR may also contain a co-receptor hinge sequence, such as a CD8 a co-receptor hinge sequence.

In embodiments, binding partners of this disclosure may comprise a constant region, e.g., an Fc region. Any isotype of constant region can be included. Binding partners that comprise a constant region may be particularly adapted for antibody-dependent cell mediated cytotoxicity (ADCC) or antibody dependent cellular phagocytosis (ADCP) and thus may function to kill targeted cells by cell-mediated responses by any of a variety of effector cells. Similarly, a constant region may be particularly adapted for enhancing complement-mediated responses.

In embodiments, a binding partner of this disclosure may be modified such that it is present in a fusion protein. In embodiments, an antigen binding segment of a binding partner may be present in a fusion protein, and/or the constant region may be a component of a fusion protein. In embodiments, a fusion protein comprises amino acids from at least two different proteins. Fusion proteins can be produced using any of a wide variety of standard molecular biology approaches, including but not necessarily limited to expression from any suitable expression vector. In embodiments, a binding partner described herein may be present in a fusion protein with a detectable protein, such as green fluorescent protein (GFP), enhanced GFP (eGFP), mCherry, and the like. In embodiments, as an alternative to an expression vector, an mRNA or chemically modified mRNA encoding any binding partner described herein can be delivered to cells such that the binding partner is translated by the cells. In embodiments, the fusion proteins comprise a binding partner disclosed herein and a cytokine. In embodiments, the cytokine is IL-2, IL-7, IL-15, IL-12, IL-18, or IL-21. In embodiments, the cytokine is modified to increase at least one therapeutic property, including but not limited to bioavailability, efficacy, extension of half-life, or other desirable properties. Examples of suitable cytokine modifications are described in Front. Immunol., 14 Oct. 2021, doi.org/10.3389/fimmu.2021, the disclosure of which is incorporated herein by reference.

In some embodiments, the binding partner of this disclosure may be fused to another antibody. For example, the binding partner of this disclosure may be fused to an antibody or fragment thereof that is an immune checkpoint inhibitor. In some embodiments, the antibody or fragment thereof that is an immune checkpoint inhibitor can be an anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.

In embodiments, binding partners described herein are used to carry drugs or toxins, and thus the binding partners may be provided as immunotoxins, or in the form of antibody-drug conjugates (ADCs).

In embodiments, agents useful in the generation of immunotoxins include enzymatically active toxins and enzymatically active fragments thereof. Suitable enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. These can be provided as components of fusion proteins or can be covalently attached to the binding partner by any suitable conjugation approach.

The binding partner may be connected to a chemotherapeutic agent by using any suitable linker to form an antibody drug conjugate (ADC). In embodiments, the linker comprises a disulfide, a hydrazine, or a thioether. In embodiments, the ADC comprises two payloads (e.g., two different chemotherapeutic agents), each linked to the binding partner by a linker. In embodiments, the ADC comprises two payloads (e.g., two different chemotherapeutic agents), each linked to the binding partner by a linker using site specific aldehyde tags. The chemotherapeutic agent may be reversibly or irreversibly attached to the binding partner.

Cleavable linkers may be particularly useful for killing bystander cells. In embodiments, a protease recognition site may be included to liberate the chemotherapeutic agent from the binding partner by operation of a protease that recognizes and cleaves at the protease recognition site. The ADC may therefore be considered to contain a prodrug.

In embodiments, binding partners of this disclosure may comprise linking sequences. As a non-limiting example, an ScFv may comprise a linker that links segments comprising paratopes to one another. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, and 20 amino acids. In an example, the linker may be the glycine-serine-alanine linker G₄SA₃ or a glycine-serine linker (G₄S)₄ linker. In embodiments, a binding partner may include a cellular localization signal, or a secretion signal. In embodiments, binding partner may comprise a transmembrane domain, and thus may be trafficked to, and anchored in a cell membrane. For secretion, any suitable secretion signal can be used, and many are known in the art.

In embodiments, the binding partners can be part of an ADC and therefore the binding partners comprise a drug. The drug can include, but is not necessarily limited to, any suitable chemotherapeutic agent. In embodiments, the ADC comprises a binding partner and a chemotherapeutic agent that is an anti-microtubule agent, an alkylating agent, or a DNA minor groove binding agent. In embodiments, the chemotherapeutic agent comprises a maytansinoid, a dolastatin, an auristatin drug analog, or a cryptophycin. In embodiments, the chemotherapeutic agent is a duocarmycin derivative, or an antibiotic, such as an enediyne antibiotic, or pyrolobenodiazepine (PBD), including dimers thereof. In embodiments, the chemotherapeutic agent is an enzyme inhibitor, such as a topoisomerase or polymerase inhibitor. In embodiments, the chemotherapeutic agent comprises doxorubicin, or a metal-containing compound, such as a platinum-containing compound, non-limiting examples of which include cisplatin, carboplatin or oxaliplatin. In embodiments, the ADC comprises a binding partner described herein, and any drug that is described in Barf and Kaptein, dx.doi.org/10.1021/jm3003203, J. Med. Chem. 2012, 55, 6243-6262, or in Wilson et al., dx.doi.org/10.1021/jm400224q, J. Med. Chem. 2013, 56, 7463-7476, Lambert and Morris, Adv Ther (2017)34:1015-1035, or Tarantino and Tolaney, Cancer Res (2022) 82 (20): 3659-3661, from which the descriptions of drugs for use as components as ADCs is incorporated herein by reference. In embodiments, the binding partner is conjugated to or otherwise includes a cytokine, including but not necessarily limited to an interleukin, including but not limited to IL-2, IL-7, IL-8, IL-15, IL-12, IL-18, or IL-21, or an interferon (IFN), to thereby provide a cytokine conjugate. In embodiments, the binding partner comprises a toxin conjugate. The toxin conjugate can include, but is not necessarily limited to Pseudomonas exotoxin A (PE), diphtheria toxin (DT), or a recombinant variant thereof. In some embodiments, the binding partner can comprise an immunotoxin agent derived from PE or DT, including but not limited to BL22, LMB-2, CAT-8015, SSIP, MR1-1, or Zemab.

For production of binding partners, any suitable expression system may be used. In general, polynucleotides encoding binding partners are used to express the binding partners in any suitable cell system, non-limiting embodiments of which include NS0 murine myeloma cells, human cell lines, and Chinese hamster ovary (CHO) cells. In embodiments, the disclosure provides a polynucleotide that can selectively hybridize to a polynucleotide encoding any CDR or combination of CDRs described herein. In embodiments, the polynucleotide selectively hybridizes to a polynucleotide encoding a heavy chain CDR1, CDR2, and CDR3 of any described binding partner. In embodiments, the polynucleotide selectively hybridizes to a polynucleotide encoding a light chain CDR1, CDR2, and CDR3 of any described binding partner. In embodiments, the polynucleotide selectively hybridizes to a polynucleotide encoding CDR1, CDR2, and CDR3 of a heavy and light chain of any described binding partner.

In embodiments, a binding partner described herein may be a component of a fusion protein. In embodiments, such as for a binding partner that is produced as a fusion protein, a peptide linker may be used. In embodiments, the peptide linker comprises any self-cleaving signal. In embodiments, the self-cleaving signal may be present in the same open reading frame (ORF) as the ORF that encodes the binding partner. A self-cleaving amino acid sequence is typically about 18-22 amino acids long. Any suitable sequence can be used, non-limiting examples of which include: T2A (EGRGSLLTCGDVEENPGP); P2A (ATNFSLKQAGDVENPGP); E2A (QCTNYALKLAGDVESNPGP) and F2A (VKQTLNFDLKLAGDVESNPGP).

To the extent any segment of a protein comprising a binding partner described herein was a component of a library, including but not necessarily limited to a phage display library or a yeast surface display library, the disclosure includes the proviso that the binding partner may be free of any segment of the library that comprises a bacteriophage or yeast amino acid sequence, including but not limited to phage coat protein or a yeast host protein, including but not limited to Aga2. Thus, in certain embodiments, the binding partner may be present in a fusion protein, but the fusion protein does not comprise bacteriophage coat protein. In embodiments, any binding partner described herein may be free of any of pIII phage coat protein, or any part of MI, fd filamentous phage, T4, T7, or λ phage protein.

In embodiments, a binding partner of this disclosure comprises a detectable label, which may be used for diagnostic or therapeutic purposes. For example, a detectable label can be used for localization of the binding partner for pathology and/or in vivo imaging approaches. In embodiments, a binding partner is conjugated to any of a variety of radioactive agents, including but not limited to a highly radioactive atom, such as In111, At211, I131, I125, Y90, Re186, Re188, Sm153, Bi212, P32, Pb212, and radioactive isotopes of Lu. In particular embodiments, such as for imaging, the binding partner may be conjugated to a radioactive atom for scintigraphic approaches, for example Tc99m (metastable technetium-99), 1123, or a spin label for nuclear magnetic resonance (NMR) imaging (also known as magnetic resonance imaging, or “MRI”), such as 1123, 1131, 1124, F19, C13, N15, O17 or Gadlinium (III) or Manganese (II). In embodiments, the radioactive agent is suitable for use in CAT scan or PET imaging. In embodiments, Indium111, Technetium99 or Iodine131 can be used for planar scans or single photon emission computed tomography (SPECT). Positron emitting labels such as Fluorine19 Iodine 123 and Iodine 124 can be used in positron emission tomography. Paramagnetic ions such as Gadlinium (III) or Manganese (II) can used in magnetic resonance imaging MRI. In embodiments, the described radioactive isotopes that are attached to a described binding partner can also be used in therapeutic approaches. In embodiments, radioactive agents or isotopes include alpha-emitting radionuclides. In embodiments, radioactive agents or isotopes include beta-emitting radionuclides. In some embodiments, the present disclosure provides an antibody of the present technology conjugated to a diagnostic or therapeutic agent. The diagnostic agent may comprise a non-radioactive label, a contrast agent (such as for magnetic resonance imaging, computed tomography or ultrasound), and/or a radioactive label which can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

The binding partner can be a polypeptide. The polypeptide can bind to the peptide conjugate/MHC complex. The three-dimensional structures of the polypeptide binding to the peptide-conjugate/MHC complex can be determined by various methods, for example, crystallography, cryoEM, or NMR. The polypeptide can contact with the peptide conjugate/MHC complex with an interface area of at least about 500, 600, 700, 800, 900, 1,000, 1,200, 1,500, 2,000 or more Å2. Surface areas of residues, buried upon the interaction between the polypeptide and the peptide conjugate/MHC complex can be calculated by PDBePISA. The polypeptide can contact with the MHC of the peptide conjugate/MHC complex with an interface area more than that of the peptide or the targeted covalent inhibitor. The polypeptide can form a binding pocket at the interface between VH and VL domains to accommodate the targeted covalent inhibitor of the peptide conjugate/MHC complex. The polypeptide may not bind to the peptide conjugate/MHC complex with a head-to-head coaxial interaction. In some cases, the polypeptide may bind to the peptide conjugate/MHC complex with a head-to-head coaxial interaction. For example, the polypeptide may bind to the peptide conjugate/MHC complex in a similar way as a TCR binding to the peptide/MHC complex. The polypeptide can bind to the peptide conjugate/MHC complex with an angle from about 10° to 60°, from about 10° to 70°, or from about 10° to 80° between the axis of the MHC and the axis of the polypeptide. The polypeptide can bind to the peptide conjugate/MHC complex with an angle of about 40° between the axis of the MHC and the axis of the polypeptide.

The polypeptide can contact the alpha 1 domain and alpha 2 domain of a heavy chain of the MHC. The polypeptide can bind to an epitope of the MHC. The epitope can comprise one or more residues from the regions comprising residues 62-66, 106-109, and/or 150-170 of the MHC. The polypeptide can bind to an epitope of the MHC, and the epitope can comprise one or more residues from the regions comprising residues 62-66, 106-109, and/or 150-170 of the HLA-A*03:01 or the HLA-A*11:01. The polypeptide can bind to an epitope of the MHC, and the epitope can comprise one or more residues selected from the group consisting of residues 62, 106, 108, 109, 158, 161, 162, 162, 165, 166, 167, 169 and 170 of the HLA-A*03:01. The polypeptide can bind to an epitope of the MHC, and the epitope can comprise one or more residues selected from the group consisting of residues 62, 106, 108, 109, 158, 161, 162, 162, 165, 166, 167, 169 and 170 of the HLA-A*03:01. The polypeptide can bind to an epitope of the MHC, and the epitope can comprise one or more residues selected from the group consisting of residues 62, 65, 66, 150, 151, 154, 155, 157, and 158 of the HLA-A*03:01. The polypeptide can bind to an epitope of the MHC, and the epitope can comprise one or more residues selected from the group consisting of residues 62, 65, 66, 150, 151, 154, 155, 157, and 158 of the HLA-A*03:01. The polypeptide can bind to an epitope of the MHC, and the epitope can comprise one or more residues selected from the group consisting of residues 62, 106, 108, 109, 154, 157, 158, 161, 162, 163, 165, 166, 167, 169 and 170 of the HLA-A*11:01. The polypeptide can bind to an epitope of the MHC, and the epitope can comprise one or more residues selected from the group consisting of residues 62, 106, 108, 109, 154, 157, 158, 161, 162, 163, 165, 166, 167, 169 and 170 of the HLA-A*11:01. The polypeptide can bind to an epitope of the MHC, and the epitope can comprise one or more residues selected from the group consisting of residues 62, 65, 66, 151, 154, 155 and 158 of the HLA-A*11:01. The polypeptide can bind to an epitope of the MHC, and the epitope can comprise one or more residues selected from the group consisting of residues 62, 65, 66, 151, 154, 155 and 158 of the HLA-A*11:01.

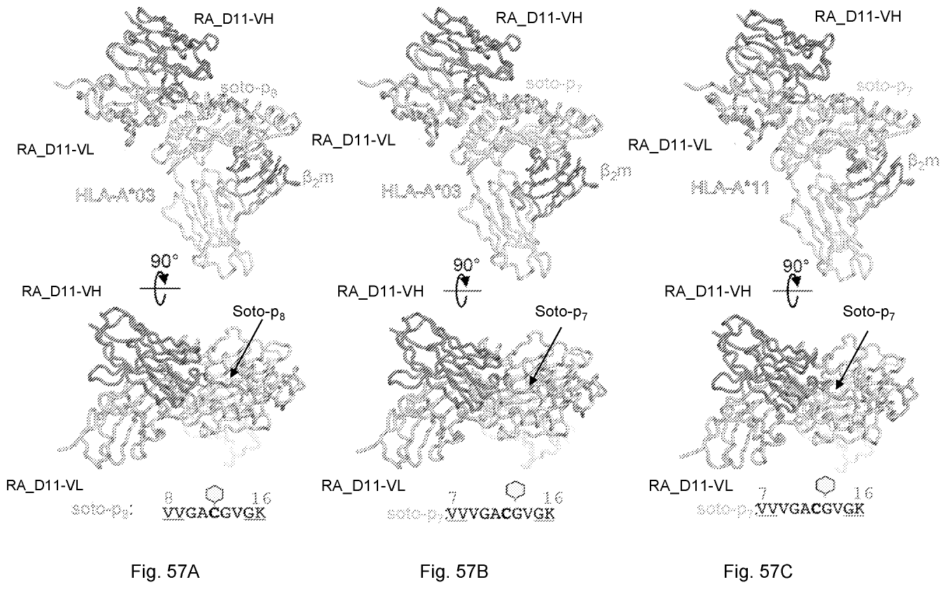

In some aspects, the present disclosure provides methods of identifying or designing polypeptides that can bind to a peptide-conjugate/MHC complex based on three-dimensional structure(s) of the peptide-conjugate/MHC complex. The three-dimensional structures of the peptide-conjugate/MHC complex may be obtained from existing complex structures of binding partners binding to the peptide-conjugate/MHC complexes (e.g., the structures provided herein). Computer-assisted methods can be used to identify or design polypeptides that can link to a targeted covalent inhibitor, or fragment thereof, that is covalently linked to the peptide of a peptide conjugate/MHC complex. In some embodiments, a method for identifying or designing polypeptides candidates that can link to a targeted covalent inhibitor comprises: (a) providing the coordinates of at least two atoms of (i) the peptide conjugate/MHC complex of the FIGS. 57A-C, 58A-B, 59A-C, 60A-B and 61. The method can further comprise (b) providing the structure of a candidate polypeptide comprising an antigen binding domain for binding to the peptide conjugate/MHC complex. The method can further comprise (c) fitting the structure of the candidate polypeptide to the at least two atoms of the peptide conjugate/MHC complex. In some cases, fitting can comprise determining interactions between one or more atoms of the antigen binding domain of the candidate polypeptide and atoms of the peptide conjugate/MHC complex. The method can further comprise (d) selecting the candidate polypeptide if it is predicted to bind to the peptide conjugate/MHC complex.

Step (a) and step (b) in the above-described computer-assisted method for designing polypeptides that can link to a targeted covalent inhibitor can occur in any order or concurrently. For example, in some embodiments, a computer-assisted method for designing polypeptides that can link to a targeted covalent comprises the steps of: (a) providing the structure of a candidate polypeptide comprising an antigen binding domain for binding to a peptide conjugate/MHC complex; (b) providing the coordinates of at least two atoms of the peptide conjugate/MHC complex and the coordinates of at least two atoms of an antigen binding domain of a polypeptide that is bound to the peptide conjugate/MHC complex of the FIGS. 57A-C, 58A-B, 59A-C, 60A-B and 61; (c) fitting the structure of the candidate polypeptide to the at least two atoms of the peptide conjugate/MHC complex and the at least two atoms of an antigen binding domain of a polypeptide that is bound to the peptide conjugate/MHC complex, wherein fitting comprises determining interactions between one or more atoms of the antigen binding domain of the candidate polypeptide and atoms of the peptide conjugate/MHC complex, and (d) selecting the candidate polypeptide if it is predicted to bind to the peptide conjugate/MHC complex.

In some embodiments of the computer-assisted method for designing polypeptides that can link to a targeted covalent inhibitor, interactions between atoms of the antigen binding domain and atoms of the peptide are determined. For example, such a method can comprise: (a) providing the structure of a polypeptide comprising an antigen binding domain bound to a peptide conjugate/MHC complex; (b) determining interactions between one or more atoms of the antigen binding domain of the polypeptide and one or more atoms of the peptide conjugate/MHC complex; (c) providing a candidate polypeptide comprising an antigen binding domain for binding to a peptide conjugate/MHC complex, wherein the antigen binding domain of the candidate polypeptide comprises one or more amino acid substitutions relative to the polypeptide comprising an antigen binding domain, wherein the one or more amino acid substitutions are of residues of the antigen binding domain of the polypeptide that comprise the one or more atoms that interact with or modulate interaction with the one or more atoms of the peptide conjugate/MHC complex; and (d) selecting the candidate polypeptide if it binds or is predicted to bind to the peptide conjugate/MHC complex with a higher affinity than the affinity of the polypeptide comprising an antigen binding domain to the peptide conjugate/MHC complex.

In some instances, the computer-assisted method for identifying or designing polypeptides that can link to a targeted covalent inhibitor comprises using a computer system. The computer system could be, for example, a programmed computer comprising a processor, a data storage system, an input device, and an output device. In some embodiments, the steps of such a methods comprise: (a) inputting into the programmed computer through said input device data comprising the three-dimensional coordinates of a subset of the atoms from or pertaining to a crystal structure of FIGS. 57A-C, 58A-B, 59A-C, 60A-B and 61, thereby generating a data set; (b) comparing, using said processor, said data set to a computer database of structures stored in said computer data storage system structures of polypeptides comprising an antigen binding domain that bind or putatively bind or that are desired to bind to a peptide conjugate/MHC complexes; (c) selecting from said database, using computer methods, structure(s) that may bind to certain peptide conjugate/MHC complexes; (d) constructing, using computer methods, a model of the selected structure(s); and (e) outputting to said output device the selected structure(s). Optionally, one or more of the selected structure(s) can be synthesized, and the synthesized structure(s) can be tested for being to a peptide conjugate/MHC complex.

A computer-readable media can be used to identify or design polypeptide candidates that comprise an antigen binding domain bound to a peptide conjugate/MHC complex. For example, in some embodiments, such a computer-readable media can contain: (a) atomic coordinate data according to the structure of any one of FIGS. 57A-C, 58A-B, 59A-C, 60A-B and 61, where the data can define the three dimensional structure or provide the structure of a polypeptide comprising an antigen binding domain bound to a peptide conjugate/MHC complex, and where the peptide conjugate of the peptide conjugate/MHC complex is a peptide covalently linked to a targeted covalent inhibitor or fragment thereof, or at least one sub-domain thereof; or (b) structure factor data for the polypeptide comprising an antigen binding domain bound to the peptide conjugate/MHC complex, where the structure factor data can be derivable from the atomic coordinate data of any one of FIGS. 57A-C, 58A-B, 59A-C, 60A-B and 61. In other embodiments, the computer-readable media can contain: (a) atomic coordinate data according to the structure of any one of FIGS. 57A-C, 58A-B, 59A-C, 60A-B and 61, where the data can define the three dimensional structure or provide the structure of a peptide conjugate/MHC complex, wherein the peptide conjugate of the peptide conjugate/MHC complex is a peptide covalently linked to a targeted covalent inhibitor or fragment thereof, or at least one sub-domain thereof; or (b) structure factor data for the peptide conjugate/MHC complex, where the structure factor data can be derivable from the atomic coordinate data of the structure of any one of FIGS. 57A-C, 58A-B, 59A-C, 60A-B and 61.

Any binding partner described herein may be fully or partially humanized. Techniques for humanization of antibodies are known in the art and can be adapted for use in the present disclosure. In embodiments, humanization may be performed, for example, by CDR-grafting. In embodiments, for humanization or to otherwise improve a characteristic of the binding partners, one or more amino acids in a variable region can be changed. In embodiments, one or more amino acids in a framework region can be changed.

The disclosure includes binding partners for use in diagnostic and therapeutic approaches. For therapeutic approaches, in certain embodiments, binding partners may be delivered as mRNA or DNA polynucleotides that encode the binding partners. It is considered that administering a DNA or RNA encoding any binding partner described herein is also a method of delivering such binding partners to an individual or one or more cells. In some embodiments, the mRNA comprises synthetic mRNA containing modified nucleotides.

In some embodiments, the modifications can include N1-methyl-pseudouridine (1 ml) nucleotide substitutions and/or 5-methylcytidine (m5C) substitutions. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus. In embodiments, a retroviral vector adapted from a murine Moloney leukemia virus (MLV) or a lentiviral vector may be used, such as a lentiviral vector adapted from human immunodeficiency virus type 1 (HIV-1).

In an embodiment, an oncolytic viral vector is used. Oncolytic viruses (oVs), including vaccinia (OVV), mediate anticancer effects by both direct oncolysis and stimulation of innate immune responses through production of damage-associated molecular patterns (DAMPs) and the presence of virus-derived pathogen-associated molecular patterns (PAMPs), leading to increased type I interferon production. Additionally, OVV-mediated oncolysis may facilitate the direct acquisition of tumor-derived antigens by host antigen-presenting cells within the tumor microenvironment, thereby leading to improved T cell priming as well as coordination of the effector phase of antitumor immune responses. In alternative embodiments, a recombinant adeno-associated virus (AAV) vector may be used. In certain embodiments, the expression vector is a self-complementary adeno-associated virus (scAAV).

Pharmaceutical formulations containing binding partners are included in the disclosure and can be prepared by mixing them with one or more pharmaceutically acceptable carriers. Pharmaceutically acceptable carriers include solvents, dispersion media, isotonic agents, and the like. The carrier can be liquid, semi-solid, e.g. pastes, or solid carriers. Examples of carriers include water, saline solutions or other buffers (such as phosphate, citrate buffers), oil, alcohol, proteins (such as serum albumin, gelatin), carbohydrates (such as monosaccharides, disaccharides, and other carbohydrates including glucose, sucrose, trehalose, mannose, mannitol, sorbitol or dextrins), gel, lipids, liposomes, resins, porous matrices, binders, fillers, coatings, stabilizers, preservatives, liposomes, antioxidants, chelating agents such as EDTA, salt forming counter-ions such as sodium; non-ionic surfactants such as TWEEN, PLURONICS or polyethylene glycol (PEG), or combinations thereof. In embodiments, a liposomal formulation comprising one or more binding partners is provided. Liposomal formulations include but are not limited to liposomal nanoparticles.

In embodiments, an effective amount of one or more binding partners is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight, and gender of the patient, desired duration of treatment, method of administration, time and frequency of administration, drug combination(s), reaction sensitivities, and/or tolerance/response to therapy.

Binding partners and pharmaceutical compositions comprising the binding partners can be administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. The compositions may be administered parenterally or enterically. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated.

In embodiments, the individual in need of a composition of this disclosure has been diagnosed with or is suspected of having cancer. In embodiments, the cancer is a solid tumor or a hematologic malignancy. In embodiments, the cancer is renal cell carcinoma, breast cancer, prostate cancer, pancreatic cancer, lung cancer (e.g. non-small-cell lung cancer), liver cancer, ovarian cancer, cervical cancer, colon cancer (or colorectal cancer), esophageal cancer, glioma, glioblastoma or another brain cancer, stomach cancer, bladder cancer, testicular cancer, head and neck cancer, thyroid cancer, adrenal cancer, melanoma or another skin cancer, any sarcoma, including but not limited to fibrosarcoma, angiosarcoma, osteosarcoma, and rhabdomyosarcoma, and any blood cancer, including all types of leukemia, lymphoma, and myeloma. In some embodiments, the individual is in need of treatment for a neuroendocrine tumor. In some embodiments, the individual is in need of treatment for any pre-neoplastic disorder, including myelodysplastic syndromes or myeloproliferative neoplasms. In embodiments, a described binding partner is used prophylactically for any of the described types of cancer. In some embodiments, the individual in need thereof has been treated with a prior treatment and the individual is refractory to the prior treatment. In some embodiments, the cancer is a relapsed or refractory cancer.

In embodiments, administering one or more binding partners, including but not necessarily in a pharmaceutical formulation, to an individual in need thereof, exhibits an improved activity relative to a control. In an embodiment, the control comprises different antibodies, a different form of the same antibodies/binding partner, or antibodies/binding partners that are delivered without adding additional agents. In embodiments, a binding partner described herein provides for improved antibody dependent cell cytotoxicity (ADCC), or for internalization (such as for an ADC), relative to a control. In embodiments, a control protein or peptide does not comprise the covalently linked molecule. The control peptide may comprise the same sequence as the experimental peptide, or if the experimental peptide comprises a mutation the control peptide may comprise the wild type sequence.

A composition of this disclosure, such as a pharmaceutical formulation, can contain only one, or more than one binding partner, and thus combinations of different binding partners are included.

In an aspect, provided herein is a method of treating a disease or disorder in a subject, the method comprising administering to the subject: (a) a targeted covalent inhibitor, and (b) a binding partner disclosed herein. In an embodiment, the disease or disorder is cancer, a fibrotic disease, or an autoimmune disease (e.g., rheumatoid arthritis). In an embodiment, the targeted covalent inhibitor targets KRAS, Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, a Cancer Testis Antigen, an E3 ligase, a DCAF, an ERV, a LINE, or a transposable element protein.

In an aspect, provided herein is a method of killing a cancer cell in a subject, the method comprising administering to the subject: (a) a targeted covalent inhibitor, and (b) a binding partner disclosed herein. In an embodiment, the targeted covalent inhibitor targets KRAS, Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, a Cancer Testis Antigen, an E3 ligase, a DCAF, an ERV, a LINE, or a transposable element protein.

In an aspect, provided herein is a method of targeting a cell that expresses EGFR, BTK, or RAS in a subject that has been treated with an EGFR, BTK, or RAS targeted covalent inhibitor, the method comprising administering to the subject a binding partner disclosed herein. In an embodiment, the subject has cancer.

In an aspect, provided herein is a method of treating a disease or disorder in a subject that has been treated with a targeted covalent inhibitor, the method comprising administering to the subject a binding partner disclosed herein. In an embodiment, the disease or disorder is an autoimmune disease or a fibrotic disease.

In an aspect, provided herein is a method of treating cancer in a subject that has been treated with a targeted covalent inhibitor, the method comprising administering to the subject a binding partner disclosed herein. In an embodiment, the targeted covalent inhibitor targets RAS, Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, a Cancer Testis Antigen, an E3 ligase, or a DCAF.

The targeted covalent inhibitor can be a non-peptide molecule described herein. The non-peptide molecule can be administered to a subject in need thereof prior to administering the binding partner described herein. The subject described herein can be a cancer patient. In some cases, the binding partner can be administered to a subject already treated with a non-peptide molecule previously. In some cases, the binding partner can be administered to a subject simultaneously or after administration of a non-peptide molecule (e.g., the targeted covalent inhibitor or the drug described herein). In some cases, the binding partner can be administered to a subject before administration of a non-peptide molecule.

In some cases, the subject described herein is refractory to a first line of treatment. For example, the subject described herein can be refractory to a first line of treatment which is a chemotherapy. For example, the subject described herein can be refractory to a non-peptide molecule such as AMG-510, osimertinib, ARS-1620, MRTX849, JNJ74699157, LY3499446, MRTX-1257, JDQ443, MRTX-1133, RMC-6291, or RMC-9805. In some cases, the subject can be further treated or administered with the binding partner described herein (e.g., antibody or fragment thereof) after relapsed from a prior treatment. In some cases, the binding partners described herein can be used to treat a relapsed or refractory cancer.

In some cases, a subject is prophylactically administered the peptide conjugate described herein prior to the subject developing a cancer, or prior to the subject being administered a drug, or both. The cancer can be due to a RAS mutation. The cancer can be due to HRAS mutation, e.g., HRAS^{G12C}, HRAS^{G12D}, HRAS^{G12R}, or HRAS^{G12S}. The cancer can be due to NRAS mutation, e.g., NRAS^{G12C}, NRAS^{G12D}, NRAS^{G12R}, or NRAS^{G12S}. The cancer can be due to KRAS mutation, e.g., KRAS^{G12C}, KRAS^{G12D}, KRAS^{G12R}, or KRAS^{G12S} In some cases, the method provided herein comprises administering a peptide conjugate to a subject prophylactically, thereby preventing the subject from developing a cancer or preventing the subject from developing a drug resistance. The subject can be a patient who would be receiving the drug described herein.

In some cases, provided herein is a method of preventing a disease or condition in a subject that will receive a drug to treat the disease or condition, the method comprising administering the peptide-drug conjugate prophylactically to a subject as a vaccine to a disease or condition treated with a drug, wherein the subject does not have the disease or condition at the time of administration, and wherein the subject will receive the drug if the subject develops the disease or condition.

In some cases, provided herein is a method of preventing drug resistance in a subject in need thereof, the method comprising administering the peptide-drug conjugate to a subject with the disease or condition, wherein the subject has previously received a drug to treat the disease or condition, and wherein the subject has not developed resistance to the drug prior to the administration of the peptide-drug conjugate, thereby preventing resistance to the drug in the subject.

In some cases, provided herein is a method of preventing drug resistance in a subject in need thereof, the method comprising administering the peptide-drug conjugate to a subject with the disease or condition, wherein the subject has not yet received a drug to treat the disease or condition prior to the administration of the peptide-drug conjugate, thereby preventing resistance to the drug in the subject.

In an aspect, provided herein is a method of enhancing immune recognition of a cell expressing a RAS or EGFR mutation in a subject that has a cancer that exhibits a RAS or EGFR mutation, the method comprising administering to the subject: (a) a RAS or EGFR inhibitor, and (b) a binding partner disclosed herein. In an embodiment, the subject has been previously treated with the RAS or EGFR inhibitor. The RAS mutation can be KRAS^{G12C} KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}. In an embodiment, the inhibitor is osimertinib, ibrutinib, neratinib, AMG-510, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. Examples of additional compounds that can covalently target a KRAS peptide containing a G12C mutation can be found in Internal Application No. PCT/IB2019/050993, Internal Application No. PCT/EP2018/083853, and U.S. Application No. U.S. Ser. No. 16/917,128, each of which is incorporated herein by reference in its entirety. In an embodiment, the inhibitor is selected from beta-lactones G12Si-1, G12Si-2, G12Si-3, G12Si-4 and G12Si-5.

In an embodiment, the cancer is renal cell carcinoma, breast cancer, prostate cancer, pancreatic cancer, lung cancer, liver cancer, ovarian cancer, cervical cancer, colon cancer (or colorectal cancer), esophageal cancer, glioma, glioblastoma, brain cancer, stomach cancer, bladder cancer, testicular cancer, thyroid cancer, adrenal cancer, head and neck cancer, melanoma, skin cancer, sarcoma, fibrosarcoma, angiosarcoma, osteosarcoma, rhabdomyosarcoma, leukemia, lymphoma, myeloma, or a neuroendocrine tumor. In an embodiment, the targeted covalent inhibitor is osimertinib, ibrutinib, neratinib, AMG-510, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. Examples of additional compounds that can covalently target a KRAS peptide containing a G12C mutation can be found in Internal Application No. PCT/IB2019/050993, Internal Application No. PCT/EP2018/083853, and U.S. Application No. U.S. Ser. No. 16/917,128, each of which is incorporated herein by reference in its entirety. In other embodiments, the targeted covalent inhibitor is selected from beta-lactones G12Si-1, G12Si-2, G12Si-3, G12Si-4 and G12Si-5.

In an embodiment, the additional therapeutic agent is a conventional chemotherapeutic agent, a modulator of T-cell costimulatory molecules, or an immune checkpoint inhibitor. In an embodiment, the additional therapeutic agent is a chemotherapeutic or an immunomodulator. In an embodiment, the immunomodulator is a checkpoint targeting agent.

In an embodiment, the additional therapeutic agent is a checkpoint targeting agent selected from the group consisting of an antagonist anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, an agonist anti-CD28 antibody. In an embodiment, the additional therapeutic agent is radiotherapy. In an embodiment, the anti-PD-1 antibody is pembrolizumab, nivolumab, or spartalizumab. In an embodiment, the anti-PD-L1 antibodies is avelumab or atezolizumab. In an embodiment, the anti-CTLA-4 antibody is ipilimumab. In an embodiment, the anti-LAG-3 antibody is relatlimab. In an embodiment, the additional therapeutic agent is adoptive immunotherapy.

In an embodiment, the immunomodulator is selected from the group consisting of a cytokine, a modified cytokine, a stem cell growth factor, a lymphotoxin, a hematopoietic factor, a colony stimulating factor (CSF), an interferon (IFN), erythropoietin, thrombopoietin, a lymphotoxin (e.g., a tumor necrosis factor (TNF)), a hematopoietic factor such as interleukin (IL), a colony stimulating factor (e.g., granulocyte-colony stimulating factor (G-CSF), granulocyte macrophage-colony stimulating factor (GM-CSF)), an interferon (e.g., interferons-alpha, -beta, -gamma, -lambda), a stem cell growth factor, and an immunomodulatory drug (IMiD/CelMoD) (e.g., lenalidomide, pomalidomide, avadomide, iberdomide, and GSPT1 degraders (e.g. CC-90009; MRT-2359)). The cytokine can be an anchored cytokine (e.g., an anchored IL-2). The anchored cytokine can be a cytokine fused a collagen-binding protein. The cytokine can be an engineered cytokine. The engineered cytokine can be a variant or mutant of a wild-type cytokine.

In an embodiment, the additional therapeutic agent is a glutaminase inhibitor, such as Telaglenastat (CB839). In an embodiment, the additional therapeutic agent is an arginase inhibitor, such as INCB001158 (numidargistat, CB-280). In an embodiment, the additional therapeutic agent is a high-affinity adenosine 2a receptor (A2aR) inhibitor, and/or low-affinity A2bR inhibitor (e.g., imaradenant, ciforadenant, inupadenant, TT-702 etrumadenant and INCB106385). In an embodiment, the additional therapeutic agent is an anti CD73 antibody or small molecule modulator (e.g., AB680, OP5244). In an embodiment, the additional therapeutic agent is a kynurenine pathway inhibitor, TDO and IDO inhibitors.

In an embodiment, the additional therapeutic agent is an AHR blocker (e.g., BAY2416964 and IK175). In an embodiment, the additional therapeutic agent is a TREX1 modulator, GTP modulator, ATP modulator, cGAMP modulator, cGAMP modulator, cGAS modulator, ENPP1 modulator, CD73 modulator, CD39 modulator, CD38 modulator, TBK1 modulator, PARP7 modulator, IRF3 modulator, or Type I IFN modulator.

Examples of additional therapeutic agents include, but are not limited to, STING agonists such as cyclic dinucleotides (CDNs), SB 11285, SNX281, GSK3745417; ENPP1 inhibitors such as MV626, SR-8314, SR-8541 (ref. 118); three-prime repair exonuclease 1 (TREX1) inhibitors; PARP7 inhibitors (e.g., RBN-23972), TLR agonists such as TLR7/TLR8 ligand imiquimod, monophosphoryl lipid A (MPL), TLR4 ligand, TLR7 agonist (e.g., LHC165); DGKa and DGKζ inhibitors; Diacylglycerol (DAG); CBL-B inhibitors: cell-permeable peptides (e.g., APN431) and CBLB-targeting small interfering RNA (siRNA) e.g., APN401) or CBLB small molecule modulators.

Examples of additional therapeutic agents include, but are not limited to, anti-androgens (e.g., Casodex, Flutamide, MDV3100, or ARN-509, Enzaluatide, apalutamide, darolutamid and abiraterone), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), BRAF inhibitor (e.g. Vemurafenib), CDK4/6 inhibitors, SHP1 inhibitors, SHP2 inhibitors (e.g. TNO155; RMC-4630; RMC-2550; RMC-4550; SHP099), PTPN22 inhibitors, PTPN1 inhibitors, PTPN2 inhibitors, CCR2 inhibitors, CXCR2 inhibitors, TORC1/TORC 2 inhibitors, PI-3K-AKT inhibitors, PARP inhibitors (e.g. Olaparib, Niraparib, Rucaparib), CDK4/6 inhibitors, CDK2 inhibitors, CDK7 inhibitors, TEAD inhibitors, alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, or BAY 43-9006), inhibitors of phosphatidylinositol 3-kinase signaling (e.g. wortmannin or LY294002), mTOR inhibitors, antibodies (e.g., rituxan), MAP4K1 inhibitor (e.g ZYF0033), 5-aza-2′-deoxycytidine, doxorubicin, vincristine, etoposide, gemcitabine, imatinib (Gleevec®), geldanamycin, dasatinib, 17-N-Allylamino-17-Demethoxygeldanamycin (17-AAG), bortezomib, carfilzomide, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (e.g., ICOS, Silmitasertib); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; RAS farnesyl protein transferase inhibitors; RAS inhibitors; RAS-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-CD22, anti CD25, anti-CD37, anti-CD38, anti-HER2, anti-CD52, anti-HLA-DR, anti Nectin-4, anti Trop2, anti-Muc1, anti-mesothelin, anti-alpha-folate, anti DLL3, anti-GPRNMB, anti-Glypican3, anti-VEGF, anti-EGFR monoclonal antibodies), immunotoxins (e.g., anti-CD33 monoclonal antibody-calicheamicin conjugate, anti-CD22 monoclonal antibody-pseudomonas exotoxin conjugate, anti-CD30 etc.), radioimmunotherapy (e.g., anti-CD20 monoclonal antibody conjugated to 111 In, 90Y, or 131I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, or the like.; Vaccines (e.g., Bacillus Calmette-Guérin (BCG), CSF1R inhibitors (e.g., pexidartinib); other class 3 receptor tyrosine kinases (RTKs), such as KIT, FLT3, and platelet-derived growth factor receptors PDGFRα and PDGFRβ.

In embodiments, the disclosure comprises administering to an individual in need thereof one or more binding partners and at least one additional agent to provide an additive effect, or a greater than additive effect such as a synergistic result. In embodiments, the described effect comprises inhibition of cancer growth, inhibition of metastasis, or other beneficial effect. An additive effect or synergistic effect may also be achieved by using a combination of at least two described binding partners.

In an aspect, provided herein is a method of detecting a peptide conjugate/MHC complex in a biological sample, the method comprising contacting the sample with a binding partner disclosed herein, wherein the peptide conjugate/MHC complex comprises a peptide conjugate formed by the covalent reaction of a targeted covalent inhibitor with a peptide. In an embodiment, the peptide is an EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) peptide. In an embodiment, the targeted covalent inhibitor is an EGFR, BTK, or RAS (e.g., KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}) inhibitor. In an embodiment, the targeted covalent inhibitor is osimertinib, ibrutinib, neratinib, AMG-510, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391. Examples of additional compounds that can covalently target a KRAS peptide containing a G12C mutation can be found in Internal Application No. PCT/IB2019/050993, Internal Application No. PCT/EP2018/083853, and U.S. Application No. U.S. Ser. No. 16/917,128, each of which is incorporated herein by reference in its entirety. In an embodiment, the targeted covalent inhibitor is selected from beta-lactones G12Si-1, G12Si-2, G12Si-3, G12Si-4 and G12Si-5. In an embodiment, the targeted covalent inhibitor is a covalent degrader of an EGFR family kinase. In an embodiment, the biological sample is blood or serum.

In an aspect, provided herein is a method of identifying a cell containing a peptide conjugate/MHC complex provided herein, the method comprising contacting the cell with a binding partner disclosed herein.

Various techniques have been developed for the production of binding partners and are included in the scope of this disclosure. In embodiments, the binding partners are produced by host cells by way of recombinant expression vectors. The present disclosure includes all polynucleotide sequences encoding the amino acid sequences described herein, expression vectors comprising such polynucleotide sequences, and in vitro cell cultures comprising such expression vectors. In embodiments, the cell cultures include prokaryotic cells or eukaryotic cells. In embodiments, the cell cultures are mammalian cells. In embodiments, the cells are CHO cells. In embodiments, the cells are HEK293 cells and their derivatives. Kits comprising the binding partners, and/or cell cultures expressing the binding partners, are provided by this disclosure. In general, the kits comprise one or more sealed containers that contain the binding partners, or cells expressing them. Instructions for using the binding partners for therapeutic and/or diagnostic purposes can be included in the kits.

Cells that are modified to express any described binding partner include but are not necessarily limited to CD4+ T cells, CD8+ T cells, Natural Killer T cells, γδ T cells, neutrophils, mucosal-associated invariant T (MAIT) cells, and cells that are progenitors of T cells, such as hematopoietic stem cells or other lymphoid progenitor cells, such as immature thymocytes (double-negative CD4−CD8−) cells, or double-positive thymocytes (CD4+CD8+). In some embodiments, the cell is optionally a totipotent, multipotent, or pluripotent stem cell, wherein optionally the stem cell has an induced stem cell phenotype, or wherein the cell is optionally a leukocyte. In embodiments, the cell is a macrophage. In some embodiments, the progenitor cells comprise markers, such as CD34, CD117 (c-kit) and CD90 (Thy-1). In embodiments, the modified cells comprise macrophages. In some embodiments, the modified cells comprise neutrophils. In some embodiments, the modified cell is a neutrophil. The described modified cells may be used therapeutically or prophylactically.

In embodiments, the disclosure provides for generation of a binding partner. This approach comprises providing a plurality of distinct binding partners, exposing the plurality of distinct (e.g., different) binding partners to one or a diversity of peptide conjugates, and selecting binding partners that bind with specificity to the peptide conjugates that contain the covalently conjugated drug or other molecule, but do not bind to the protein or peptide that does not comprise the covalently conjugated drug or other molecule. As described above, this approach can be performed on a manner that either does, or does not, require the amino acid sequence of the protein or peptide to be part of the antigenic determinant. The described approach can be used to select binding partners that are specific for presentation of a peptide conjugate as a component of any MHC complex.

In embodiments, binding partners described herein and as otherwise will be apparent by those skilled in the art, can be used to determine whether or not a particular drug or other molecule forms a covalent interaction with a protein or peptide. Thus, the disclosure provides for exposing protein or peptide substrates to drug candidates and using the binding partners described herein or as identified as described herein to determine whether or not the drug forms a covalent interaction with the pertinent substrate. This determination can be made based on whether or not the binding partner binds to the protein or peptide that has been covalently attached to the drug. This approach can be used in lieu of currently available techniques, such as mass spectroscopy and the like.

In an embodiment, the disclosure comprises a method comprising treating an individual in need of said treatment, wherein the individual is optionally in need of treatment for cancer, the method comprising administering to the individual a covalent drug, and administering to the individual a described binding partner that binds with specificity to a peptide that is covalently linked to the covalent drug (a peptide conjugate) wherein optionally the specificity of the binding partner is for the peptide conjugate in an HLA complex. The drug and the binding partner may be administered concurrently or sequentially. In an embodiment, the drug is administered to the individual before the binding partner to thereby form a peptide conjugate that is present in an HLA context, after which the binding partner is administered and binds to the peptide conjugate in the HLA context, and wherein said binding may kill or inhibit growth of the cells that comprise the peptide conjugate in the HLA complex.

In another embodiment the disclosure comprises generating a neoantigen that comprises a covalently attached drug. The method of generating the neoantigen comprises administering to an individual or to cells in vitro a covalent drug, such that the drug is covalently bound to a peptide to thereby produce a neoantigen comprising a peptide conjugate. The cells in which the neoantigen is generated may be any type of cancer cells, non-limiting examples of which are described herein. The peptide may be introduced into cells, or may be processed from an intact protein by intracellular protein processing. The covalent drug may initially bind to an intact protein, or the covalent drug may initially bind to a peptide. The method may further comprise identifying the neoantigen that comprises the covalently bound drug. Identifying the neoantigen can also include identifying the neoantigen as a component of a particular peptide conjugate-HLA complex. Thus the particular HLA complex in which the neoantigen is present may also be identified. In an embodiment HLA complexes comprising the neoantigen can be separated from cells, purified to any degree of purity, and the neoantigen amino acid sequence can be determined using any suitable technique, a non-limiting example of which is mass spectrometry.

In another aspect the disclosure provides a method of developing binding partners that bind with specificity to the same peptide conjugate presented by a particular HLA type, or a plurality of HLA types. This approach can be implemented using neoantigens that comprise a covalently attached drug generated as described above. Alternatively, samples from individuals who have received a covalent drug may be obtained and used for screening binding partners. Thus, a method provided by the present disclosure comprises providing isolated peptide-conjugate HLA complexes, or cells expressing an HLA in a complex with a peptide conjugate, and screening binding partners to determine binding partners that bind with specificity to the peptide-conjugate HLA complexes. A plurality of binding partners can be screened, such as by using phage or yeast display libraries. Binding partners can be identified by preferential binding to the peptide-conjugate HLA complexes. The preferential binding can be relative to binding to the same peptide conjugate in the same HLA complex but without the covalent drug, or by preferential binding to the peptide-conjugate HLA complex relative to binding to the free covalent drug, or a combination thereof.

In another aspect, the present disclosure provides a method of identifying and isolating T cells that recognize the particular drug-peptide conjugate/MHC complex. This method can be implemented using peripheral mononuclear blood cells (PBMCs, e.g., lymphocytes and other leukocytes) obtained from patients previously treated with a targeted covalent inhibitor. Samples from subjects may be obtained and cell-surface marker techniques well known by those in the art can be used to isolate specific T cells. For example, the specific T cells can be isolated or sorted out by contacting drug-peptide conjugate/MHC complex with the T cells.

With respect to developing binding partners that bind with specificity to a plurality of HLA types, the disclosure includes adapting the procedure described above, but using a plurality of different HLAs comprising the same peptide conjugate to screen binding partners. Using this approach facilitates identification of single binding partners that can bind to the same peptide conjugate when presented in different HLA types. Such binding partners expand members of the patient population that are eligible to be treated with a single identified binding partner. In non-limiting embodiments the disclosure provides for identification of single binding partners that can bind with specificity to the same peptide conjugate that is presented by HLA-A*02:01 and at least one other HLA type. In embodiments the disclosure provides for binding partners that can bind with specificity to the same peptide conjugate presented by a combination of HLA-A*02:01, HLA-A*03:01 and HLA-A*11:01.

In embodiments, an individual who is treated with a described binding partner has a condition that is resistant to treatment with the covalent drug that forms part of a peptide conjugate.

In embodiments, binding partners of this disclosure may be used in any immunological diagnostic test, including but not limited to the imaging approaches described above. In embodiments, one or more binding partners described herein can be used as a component in any form of, for example, enzyme-linked immunosorbent assay (ELISA) assay, including but not limited to a direct ELISA, a sandwich ELISA, a competitive ELISA, and a reverse ELISA. In embodiments, one or more binding partners described herein can also be incorporated into an immunodiagnostic device, such as a microfluidic device, a lateral flow device, and the like. The binding partners may also be used in, for example, Western blots and immunoprecipitation assays.

The following Examples are intended to illustrate but not limit the disclosure. In embodiments, antibodies described in Example 3 have different properties relative to those described in Example 1. Other differences between binding partners will be apparent from the Examples and their accompanying figures. The different properties include, but are not necessarily limited to, specificity for a drug conjugate displayed in the context of a specific MHC type. Thus, binding partners may exhibit different binding partners when a peptide conjugate is in a particular MHC complex.

### EXAMPLE 1

This Example provides a description of the identification and characterization of binding partners that bind with specificity to ARS-1620, which forms a covalent interaction with KRAS^{G12C}.

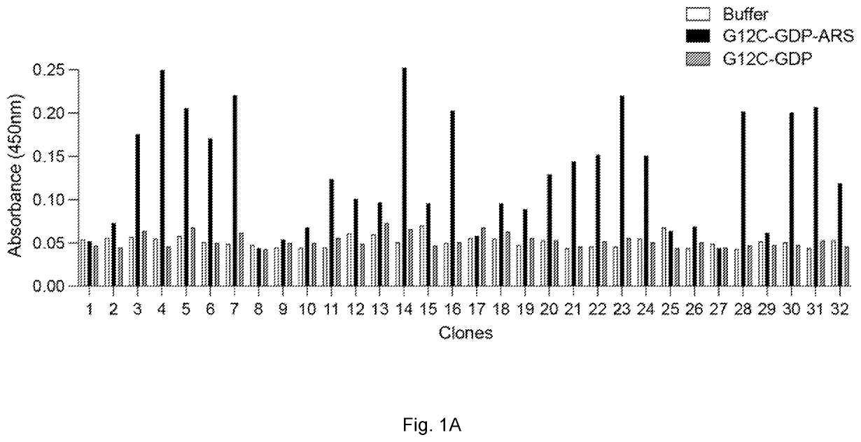

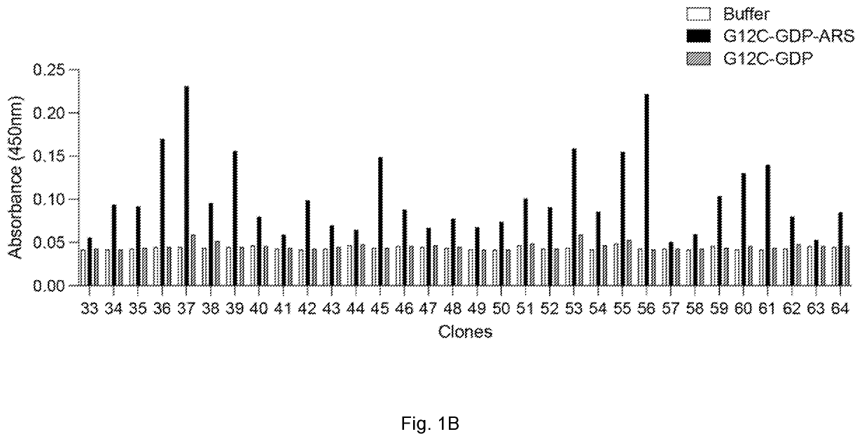

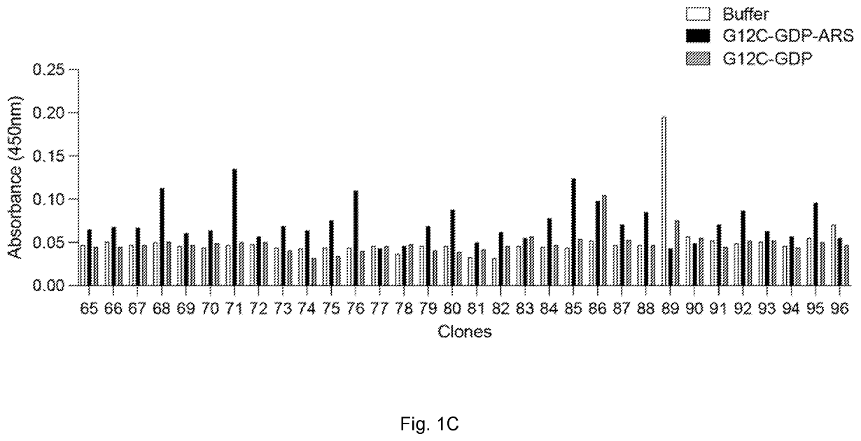

In particular, FIG. 1 demonstrates phage ELISA of phage-displayed antibody clones.

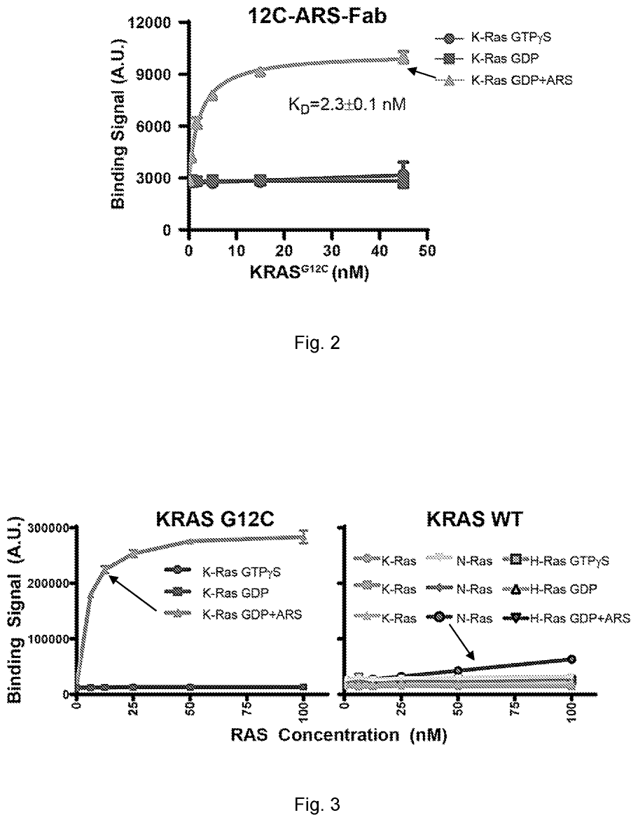

Binding to KRAS^{G12C}-GDP and KRAS^{G12C}-GDP-ARS-1620 conjugate was determined. Buffer denotes binding signal to the wells that did not contain KRAS^{G12C} From these candidates, four different antibodies were identified. Among these, 12C-ARS-Fab59 showed high affinity binding to KRAS^{G12C}-GDP covalently bound to ARS-1620. The results are presented in FIG. 2, which shows 12C-ARS Fab59 binding to KRAS^{G12C} in the GTPγS- or GDP-bound nucleotide state with or without ARS-1620, as characterized by the bead binding assay (PMID: 33358997). FIG. 3 also demonstrates that 12C-ARS-Fab59 specifically binds KRAS^{G12C}-GDP conjugated to ARS-1620. In particular, 12C-ARS-Fab binding to KRAS^{G12C} (left) or WT RAS isoforms (right) in the GTPγS- or GDP-bound nucleotide state with or without ARS-1620 conjugation, is shown as indicated.

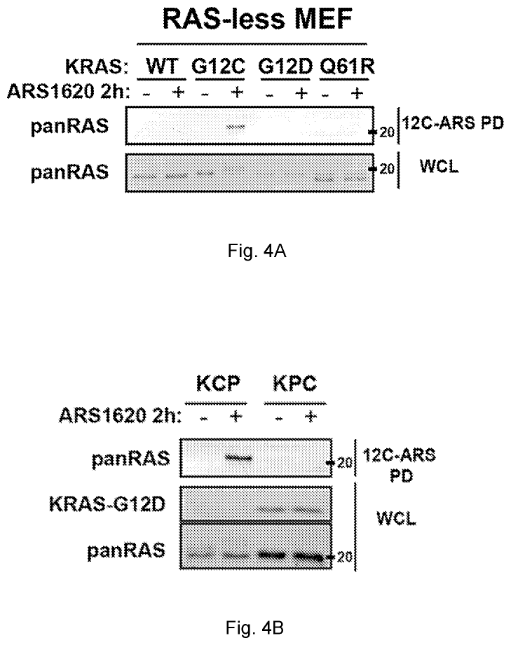

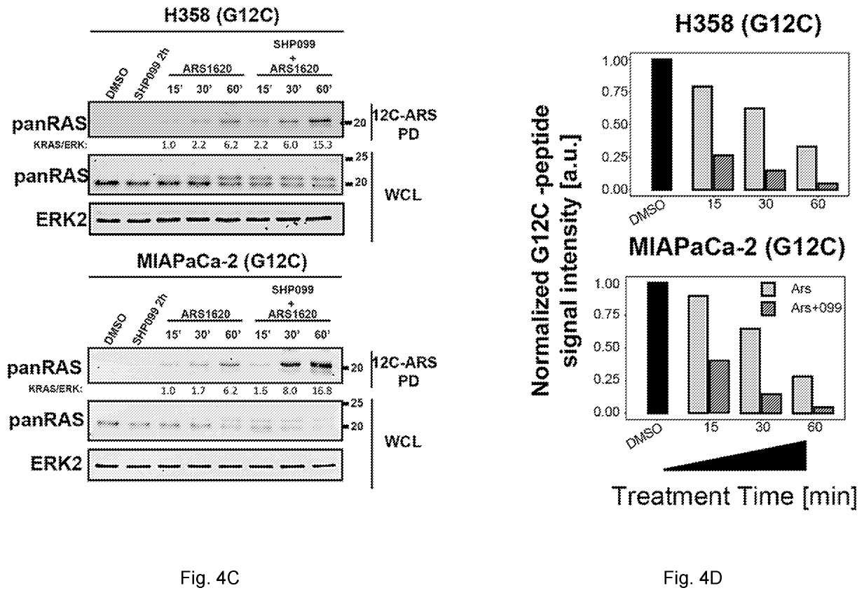

FIG. 4 demonstrates the use of 12C-ARS-Fab59 to measure ARS-1620/KRAS^{G12C} adducts by pull-down assays from lysates prepared from cell lines. To produce the data shown in FIG. 4, immunoblots were performed on whole cell lysates and 12C-ARS Fab-pull-downs (PD) from RAS-less MEFs reconstituted with the indicated KRAS mutants (4A) and from KCP (Kras^{G12C}; Tp53^{R172H}; Pdx-Cre) mouse pancreas cancer cells (4B), treated in the presence or absence of ARS-1620. FIG. 4C shows whole cell lysates and 12C-ARS Fab pull-downs (PD) from H358 and MIAPaCa-2 cells, treated as indicated, which were subjected to SDS-PAGE and immunoblotting with anti-pan RAS and anti-ERK2 antibodies, the latter as a loading control. FIG. 4D shows ARS-adduct formation in samples from 4C, quantified by LC/MS-MS assay. ARS-1620 and SHP099 concentrations were 10 μM in all panels.

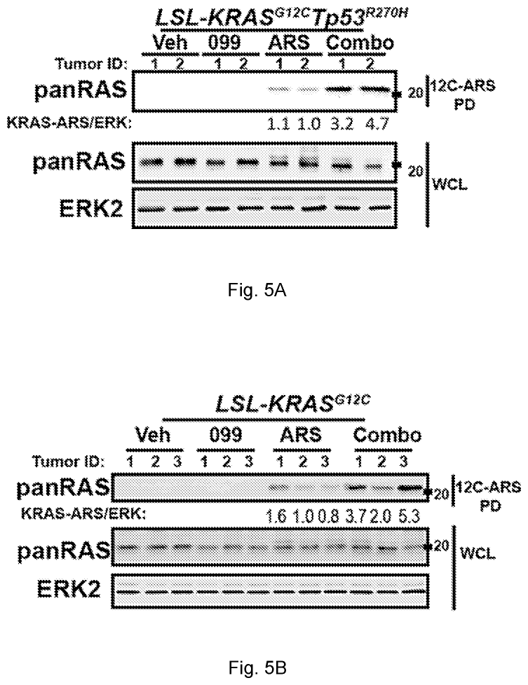

FIG. 5 shows that 12C-ARS-Fab59 can be used to measure the engagement of ARS-1620 to mutant KRAS by pull-down assay with lysates prepared from animal tissues. In particular, in FIGS. 5A-B, anti-pan RAS and anti-ERK2 (loading control) immunoblots of lysates and 12C-ARS Fab pull-downs (PD) from LSL-KRAS^{G12C}-Tp53^{R270H} (A) and LSL-KRAS^{G12C} (B) tumors after 3 days of oral gavage with ARS-1620 (200 mg/kg/d) alone or with the SHP2 inhibitor SHP099 (75 mg/kg/d) are shown.

To produce the foregoing results, the following materials and methods were used.

### RAS Nucleotide Exchange and Generation of ARS-1620-Conjugated RAS

Purified RAS (1-174) proteins containing a 6×HIS-tag and an AVI-tag⁽¹⁾, used in the binding experiments and phage display selections, were prepared by diluting stock protein (typically containing 20-100 μM RAS) 25-fold with 20 mM Tris-Cl buffer pH 7.5 containing 5 mM EDTA, 0.1 mM DTT, and 1 mM (final concentration) of nucleotide (GDP or GTPγS). For generating ARS-bound RAS, ARS-1620 (final concentration: 100 μM) was added during the nucleotide exchange reaction of RAS along with GDP. Samples were incubated at 30° C. for 30 minutes. MgCl₂ was then added to a final concentration of 20 mM to quench the nucleotide exchange reaction, and the solution was incubated on ice for at least 5 minutes prior to use.

### Selection of Phage-Displayed Antibody Fragments Against ARS-Bound KRAS^{G12C}

General procedures for the development of Fabs against purified protein targets have been described⁽²⁾. Four rounds of phage display library selection were performed, with biotinylated KRAS(G12C)-GDP+ARS-1620 at 100 nM, 100 nM, 50 nM, and 20 nM in the first, second, third and fourth rounds, respectively. The first round recovered clones that bound to KRAS^{G12C}-GDP+ARS-1620; the second round recovered clones that bound to KRAS^{G12C}-GDP+ARS-1620, previously pre-cleared with KRAS^{G12C}-GDP; the third round recovered clones that bound to KRAS^{G12C}-GDP+ARS-1620, previously pre-cleared with KRAS^{G12C}-GTP. The final round recovered clones that bound to KRAS^{G12C}-GDP+ARS-1620, previously precleared with KRAS^{G12C}-GDP. Phage captured on beads were eluted in 100 μl of 0.1 M Gly-HCl (pH 2.1) and immediately neutralized with 35 μl of 1M Tris-Cl (pH 8). Recovered clones were analyzed by phage ELISA and DNA sequencing, as described⁽²⁾.

### Bead Binding Assays

General methods have been previously described (3). Fifty microliters (50 μl) of M280 streptavidin beads (Thermo Fisher) were incubated with 100 μl of biotinylated 12C-ARS Fab, at 30 nM or 4 nM. Ligand-free streptavidin on the beads was then blocked by adding excess biotin. Beads were washed with supplemented TBST (50 mM Tris pH7.5, 150 mM NaCl, 20 mM MgCl₂, 0.1 mM DTT, 0.05% Tween-20) and dispensed into wells of a 96-well U bottom plate (Greiner). Beads were then incubated at 1:1 ratio with purified RAS proteins diluted in supplemented TBS (50 mM Tris pH7.5, 150 mM NaCl, 20 mM MgCl₂, 0.1 mM DTT) at 2× the concentration stated for the titration curve for 30 minutes at room temperature. Beads containing bound Fab and RAS were transferred to the wells of a 96-well filter plate (Millipore, MSHVN4550) and washed twice with supplemented TBST before incubating with Neutravidin-Dylight 650 (Thermo Fisher Scientific) for 30 minutes at 4° C. The beads were washed twice with supplemented TBST before resuspension in supplemented TBS for flow cytometry using an iQue screener (Sartorius). The median signal intensity in the Dylight650 channel for the 75-95th percentile population was taken as binding signal to the target. K_{D} was calculated by fitting the binding signals to a 1:1 binding model.

### Expression, Purification, and Characterization of Recombinant Fabs

Phage display vectors were converted into Fab expression vectors that contain a substrate tag for the biotin ligase BirA at the carboxyl terminus of the heavy chain. Fabs were expressed in E. coli strain 55244 (ATCC), and were purified by protein G affinity chromatography, followed by cation exchange chromatography, as described (2). Purified Fabs were biotinylated in vitro using purified BirA. Approximately 2-5 mg of purified Fabs were obtained routinely from a 1 L bacterial culture. SDS-PAGE showed that Fabs were >90% pure.

### KRAS^{G12C}-Adduct Assays

Cells cultured in 6-well plates were treated with ARS-1620 and/or SHP099 as described in the Figures. Cells were lysed by incubation in GTPase lysis buffer (25 mM Tris-Cl pH7.2, 150 mM NaCl, 5 mM MgCl₂, 1% NP-40 and 5% glycerol), supplemented with protease inhibitors and phosphatase inhibitors on ice for 15 minutes immediately before analysis. After centrifugation for 15 minutes at 15,000 g, supernatants were collected and incubated with streptavidin (SA) agarose resin (Thermo Fisher Scientific) for 1 hour at 4° C., followed by a brief centrifugation, to decrease non-specific binding to the resin. Pre-cleared lysates were incubated with biotinylated 12C-ARS-Fab bound to SA agarose for 1.5 hours at 4° C. while rotating. Agarose beads were then washed twice with GTPase lysis buffer, boiled in 1×SDS-PAGE sample buffer, and subjected to immunoblotting with a pan-RAS antibody (Millipore).

### Immunoblotting

Whole cell lysates were generated in modified radioimmunoprecipitation (RIPA) buffer (50 mM Tris-HCl pH 8.0, 150 mM NaCl, 2 mM EDTA, 1% NP-40, and 0.1% SDS, without sodium deoxycholate), supplemented with protease (40 ug/ml PMSF, 2 ug/ml antipain, 2 ug/ml pepstatin A, 20 ug/ml leupeptin, and 20 ug/ml aprotinin) and phosphatase (10 mM NaF, 1 mM Na₃VO₄, 10 mM β-glycerophosphate, and 10 mM sodium pyrophosphate) inhibitors. After clarification of debris by centrifugation in a microfuge, samples were quantified with the DC Protein Assay Kit (Bio-Rad). Total lysate protein was resolved by standard SDS-PAGE and transferred in 1× transfer buffer and 15% methanol. Membranes were incubated with their respective primary and secondary antibodies labeled with IRDye (680 nm and 800 nm) and then visualized by using a LICOR device. Monoclonal pan-RAS antibody (clone Ab-3; OP40-100UG; 1:1000) was obtained from Millipore, and mouse monoclonal ERK-2 (D2: sc-1647; 1:1000) was purchased from Santa Cruz Biotechnology.

### LC/MS-MS Assay for ARS Binding to KRAS^{G12C}

Cells (5×10⁵) were treated with the indicated compounds for the times listed and subsequently washed twice with PBS and prepared for protein extraction and LC/MS-MS analysis, as described (4). LC/MS-MS was performed at the PCC Proteomics Shared Resource at NYU School of Medicine.

Similar methods were used to obtain the results described in Example 2.

The antibodies described in this Example are as follows:

### Exemplary Antibody Clones Binding to the KRAS(G12C)-ARS-1620 Conjugate:

CDR residues (Kabat numbering) in bold.

12C-ARS-Fab59
V_{L}:
DIQMTQSPSSLSASVGDRVTITCRASQSVSSAVAWYQQKPGKAPKLLIYSASSLY
SGVPSRFSGSRSGTDFTLTISSLQPEDFATYYCQQDWYFPITFGQGTKVEIK
V_{H}:
EVQLVESGGGLVQPGGSLRLSCAASGFTFSSYYIHWVRQAPGKGLEWVASISPSS
GSTYYADSVKGRFTISADTSKNTAYLQMNSLRAEDTAVYYCARYGGRSYWQKQD
SYFYQHGLDYWGQGTLVSS
12C-ARS-Fab56
V_{L}:
DIQMTQSPSSLSASVGDRVTITCRASQSVSSAVAWYQQKPGKAPKLLIYSASSLY
SGVPSRFSGSRSGTDFTLTISSLQPEDFATYYCQQSSSSLITFGQGTKVEIK
V_{H}:
EVQLVESGGGLVQPGGSLRLSCAASGFTFSSSSIHWVRQAPGKGLEWVASISSYS
GYTSYADSVKGRFTISADTSKNTAYLQMNSLRAEDTAVYYCARSYSYSEFRYYYSG
QGMDYWGQGTLVSS
12C-ARS-Fab30
V_{L}:
DIQMTQSPSSLSASVGDRVTITCRASQSVSSAVAWYQQKPGKAPKLLIYSASSLY
SGVPSRFSGSRSGTDFTLTISSLQPEDFATYYCQQSSSSLITFGQGTKVEIK
V_{H}:
EVQLVESGGGLVQPGGSLRLSCAASGFTFSSSSIHWVRQAPGKGLEWVASISSSS
GSTSYADSVKGRFTISADTSKNTAYLQMNSLRAEDTAVYYCARSNYGWRWHLVG
MDYWGQGTLVTVSS
12C-ARS-Fab85
V_{L}:
DIQMTQSPSSLSASVGDRVTITCRASQSVSSAVAWYQQKPGKAPKLLIYSASSLY
SGVPSRFSGSRSGTDFTLTISSLQPEDFATYYCQQSSSSLITFGQGTKVEIK
V_{H}:
EVQLVESGGGLVQPGGSLRLSCAASGFTFSSSSIHWVRQAPGKGLEWVASISSSS
GSTSYADSVKGRFTISADTSKNTAYLQMNSLRAEDTAVYYCARSPYVYYWYMVGF
DYWGQGTLVTVSS

This reference listing pertains to Example 1.
1. Spencer-Smith R, Koide A, Zhou Y, Eguchi R R, Sha F, Gajwani P, Santana D, Gupta A, Jacobs M, Herrero-Garcia E, Cobbert J, Lavoie H, Smith M, Rajakulendran T, Dowdell E, Okur M N, Dementieva I, Sicheri F, Therrien M, Hancock J F, Ikura M, Koide S, O'Bryan J P. Inhibition of RAS function through targeting an allosteric regulatory site. Nat Chem Biol. 2017; 13(1): 62-8. doi:10.1038/nchembio.2231. PubMed PMID: 27820802; PMCID: 5193369.
2. Fellouse F A, Esaki K, Birtalan S, Raptis D, Cancasci V J, Koide A, Jhurani P, Vasser M, Wiesmann C, Kossiakoff A A, Koide S, Sidhu S S. High-throughput generation of synthetic antibodies from highly functional minimalist phage-displayed libraries. J Mol Biol. 2007; 373(4): 924-40. Epub 2007 Sep. 11. doi:10.1016/j.jmb.2007.08.005. PubMed PMID: 17825836.
3. Nishikori S, Hattori T, Fuchs S M, Yasui N, Wojcik J, Koide A, Strahl B D, Koide S. Broad ranges of affinity and specificity of anti-histone antibodies revealed by a quantitative Peptide immunoprecipitation assay. J Mol Biol. 2012; 424 (5): 391-9. Epub 2012 Oct. 9. doi: 10.1016/j.jmb.2012.09.022. PubMed PMID: 23041298; PMCID: 3502729.
4. Patricelli M P, Janes M R, Li L S, Hansen R, Peters U, Kessler L V, Chen Y, Kucharski J M, Feng J, Ely T, Chen J H, Firdaus S J, Babbar A, Ren P, Liu Y. Selective Inhibition of Oncogenic KRAS Output with Small Molecules Targeting the Inactive State. Cancer Discov. 2016; 6(3): 316-29. Epub 2016 Jan. 8. doi:10.1158/2159-8290.Cd-15-1105. PubMed PMID: 26739882.
5. doi.org/10.1084/jem.20201414

### EXAMPLE 2

This Example provides a description of binding partners that bind with specificity to AMG-510 that is covalently linked to peptides.

To produce the results described in this Example, some methods as described in Example 1 were adapted. For this Example, AMG-510 (purchased from Selleckchem) was conjugated to a peptide corresponding to KRAS(G12C) residues 4-18:

H2N-YKLVVVGACGVGKSA(dPEG4)
(K-long chain Biotin)-amide

and a poly-Ser peptide containing a central Cys:

H2N-SSSSCSSSSW(K-long chain Biotin)-amide.

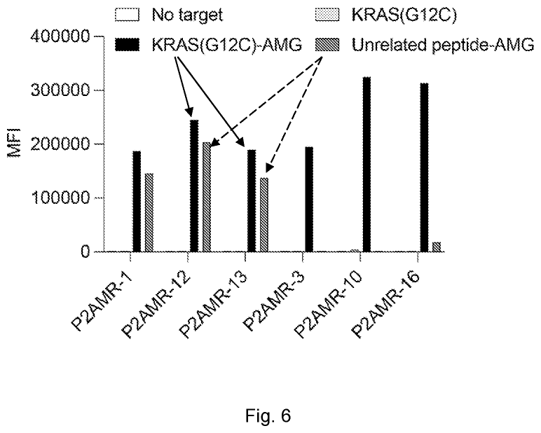

A human single-chain Fv yeast-display library was sorted using these peptides as targets by using established methods (1-3). After rounds of library sorting, individual clones were screened. We developed three antibodies that bound to AMG-510 conjugated to both KRAS(G12C) and poly-Ser peptide (FIG. 6). Consequently, these antibodies recognize predominantly the AMG-510 moiety but not the peptide moiety of the conjugates. Additionally, we developed other clones that are selective to AMG-510 conjugated to the KRAS(G12C) peptide.

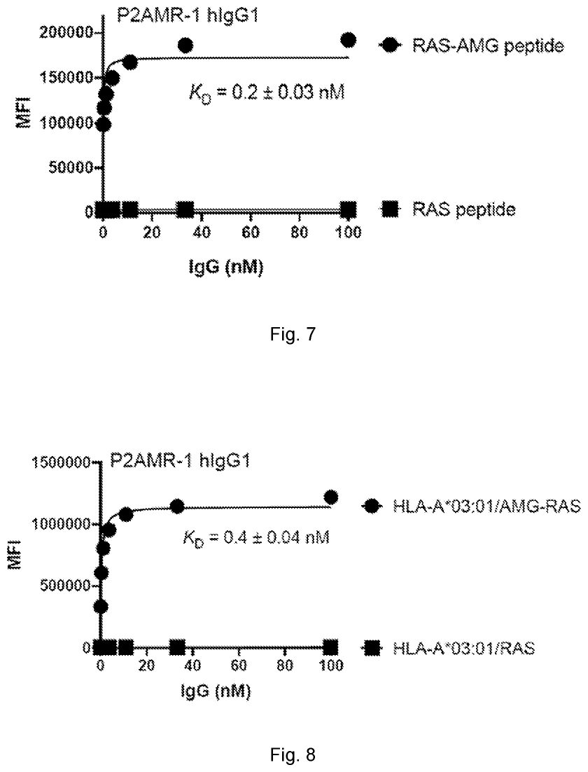

One such clone, P2AMR-1 was then produced in the format of human IgG1 and further characterized. It bound to AMG-510 conjugated to the KRAS(G12C) peptide with high apparent affinity in a bead binding assay (FIG. 7) (4). The antibody clone also bound tightly to AMG-510 conjugated a shorter KRAS(G12C) peptide, VVGACGVGK, in the context of HLA-A*03:01 (BioLegend Flex-T) (FIG. 8).

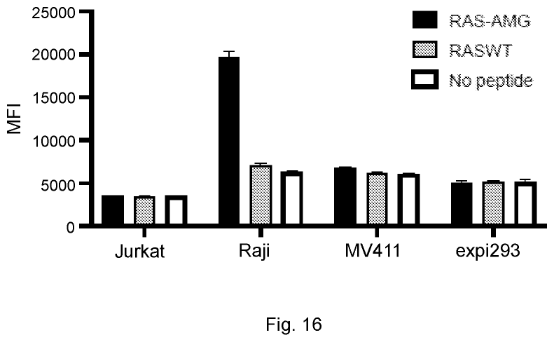

P2AMR-1 detected AMG-510 conjugated to KRAS(G12C) peptide that had been added to Raji cells, which are known to express HLA-A*03:01. By contrast, P2AMR-1 did not detect KRAS (wild type) peptide loaded in the same manner (FIG. 16). In addition, P2AMR-1 did not bind to AMG-510 conjugated to the KRAS(G12C) peptide added to cells that are not known to express HLA-A*03:01, e.g., MV4-11 and Expi293 cells (FIG. 16).

These results demonstrate that that the presently provided antibodies, which represent binding partners of this disclosure, recognize the AMG-510 moiety in a manner agnostic of the conjugation partner, and they suggest that our antibodies and their derivatives can be used to identify cells that present AMG510-KRAS(G12C) peptide conjugate on MHC molecules on the cell surface.

More generally, these results suggest methods for targeting any cells that harbor intracellular targets that form covalent adducts with small molecule ligands.

This example demonstrates the following non-limiting binding partners, restricted to AMG-510 covalent modifications of the described substrates. CDR residues (Kabat scheme) are shown in bold.

P2AMR-1
V_{L}:
QSVLIQPRSVSGSPGQSVTISCTGTSSDVGGYNYVSWYQQHPGKAPKLMIYDVS
KRPSGVPDRFSGSKSGNTASLTVSGLQAEDEADYYCGSYADTDTIVFGTGTKLTVL
V_{H}:
QVQLVQSEPEVKKPGSSVKLSCKASGGTFSTDAITWVRQAPGQGLEYMGGIIPL
LDSVDYAQRFQGRVTVSADKSTGTAYMEVRSLGSEDTAKYYCAKWSSVDTGLDY
WGQGTLVTVSS
P2AMR-12 (this clone has only the heavy chain)
V_{H}:
QVQLQESGPGLVKPSETLSLTCTVSGDSIINDPHYWGWIRQSPGKGLEWIGSTSH
SGHTYFNPSLKSRVSMSIDVAKNQFSLNVRSVTAADTAVYYCARMRYYYSGTYPV
YYFDYWGQGTLVTVSS
P2AMR-13
V_{L}:
SYVLTQPPSASGTPGQRVTISCSGSSSNIGSNFVSWYQQLPGTAPKLLISSNNQRP
SGVPDRFSGSKSDTSASLAISGLQSEDEADYYCAAWDDSLNGPVFGGGTQLTVL(
V_{H}:
QVQLVQSEAEVKKPGSSVKVSCKASGGTFSRYGVSWVRQAPGQGLEWMGGIIP
MFGTANYAQKFQGRVTITADESTSTAYMELRSLRSEDTAVYYCARGDNSAYSDAF
NIWGQGTMVTVSS

This reference listing pertains to Example 2.
1. Chao G, Lau W L, Hackel B J, Sazinsky S L, Lippow S M, Wittrup K D. Isolating and engineering human antibodies using yeast surface display. Nat Protoc. 2006; 1 (2): 755-68. Epub 2007 Apr. 5. doi:10.1038/nprot.2006.94. PubMed PMID: 17406305.
2. Feldhaus M J, Siegel R W, Opresko L K, Coleman J R, Feldhaus J M, Yeung Y A, Cochran J R, Heinzelman P, Colby D, Swers J, Graff C, Wiley H S, Wittrup K D. Flow-cytometric isolation of human antibodies from a nonimmune Saccharomyces cerevisiae surface display library. Nat Biotechnol. 2003; 21 (2): 163-70. Epub 2003 Jan. 22. doi: 10.1038/nbt785nbt785. PubMed PMID: 12536217.
3. Hattori T, Taft J M, Swist K M, Luo H, Witt H, Slattery M, Koide A, Ruthenburg A J, Krajewski K, Strahl B D, White K P, Farnham P J, Zhao Y, Koide S. Recombinant antibodies to histone post-translational modifications. Nat Methods. 2013; 10 (10): 992-5. doi: 10.1038/nmeth.2605. PubMed PMID: 23955773; PMCID: 3828030.
4. Nishikori S, Hattori T, Fuchs S M, Yasui N, Wojcik J, Koide A, Strahl B D, Koide S. Broad ranges of affinity and specificity of anti-histone antibodies revealed by a quantitative Peptide immunoprecipitation assay. J Mol Biol. 2012; 424 (5): 391-9. Epub 2012 Oct. 9. doi: 10.1016/j.jmb.2012.09.022. PubMed PMID: 23041298; PMCID: 3502729.

### EXAMPLE 3

This Example describes antibodies that bind to peptide-drug conjugates, but only in the context of specific MHC display of the described peptide-drug conjugates. The antibodies were produced as follows.

### Antigen Preparation

KRAS(G12C) peptides ((H2N-VVGACGVGK-OH and H2N-VVVGACGVGK-OH) were reacted with AMG-510 (Selleckchem) and loaded onto Flex-T HLA-A*03:01 and Flex-T HLA-A*11:01 (produced by Biolegend), or onto HLA-A*03:01 and HLA-A*11:01 produced in house. KRAS (WT) peptide ((H2N-VVGAGGVGK-OH) was loaded onto the HLA molecules in the same manner. EGFR peptide (H2N-QLMPFGCLL-OH) was reacted with osimertinib (Selleckchem) and loaded onto Flex-T HLA-A*02:01 or HLA-A*02:01 produced in house. As a control, the same peptide was reacted with beta-mercaptoethanol and loaded onto the HLA molecule. BTK peptide (H2N-YMANGCLLNY-OH was reacted with Ibrutinib (Selleckchem) and loaded onto Flex-T HLA-A*01:01 or HLA-A*01:01 produced in house. As a control, the same peptide was reacted with beta-mercaptoethanol and loaded onto the HLA molecule. The peptide-loaded HLA mixtures prepared with Flex-T HLA proteins were used without further purification. The peptide-loaded HLA mixtures prepared with HLA samples prepared in house were further purified using size-exclusion chromatography with a Superdex S200 column.

### Antibody Phage-Display Library Sorting

Sorting of an antibody phage-display library was performed as described previously (1). Briefly, a phage-display library was first sorted with all four antigens at 100 nM in the first round, followed by sorting with a single antigen at 100, 50, and 20 nM in the second, third, and fourth rounds, respectively. To enrich for clones with the desired specificity, counterselection was performed using KRAS (WT) peptide-loaded MHC molecules or beta-mercaptoethanol-treated peptide-loaded MHC molecules in the second, third, and fourth rounds.

Binding of individual phage clones were tested using the multiplex bead binding assay (2).

### Antibody Yeast-Display Library Sorting and Clone Characterization

Display of antibody clones in the form of single-chain Fv (scFv) on the yeast surface, library sorting using fluorescence-activated cell sorting, and characterization of individual clones were performed essentially as described previously (Hattori et al. PMID 23955773; Cao et al. PMID 17406305).

### Deep Mutational Scanning

Deep mutational scanning was performed following general procedures published previously (PMID 32841599). A yeast-display library, in the scFv format, contained variants in which a single position was diversified with the NNK codon. A yeast display library was subjected to FACS using an antigen of interest to enrich a pool of clones that bound the antigen and a pool of clones that did not bind the antigen. The DNA sequences of the enriched pools were determined, and amino acid substitutions were deduced.

### Antibody Production

The genes encoding selected antibody clones were transferred from the phage-display vector to IgG expression vectors (pFUSEss-CHIg-hG1 and pFUSE2ss-CLIg-hK, InvivoGen), and IgG proteins were produced using the ExpiCHO cell line (Thermo Fisher) and purified using a Protein Capture Devices with Protein A (GORE).

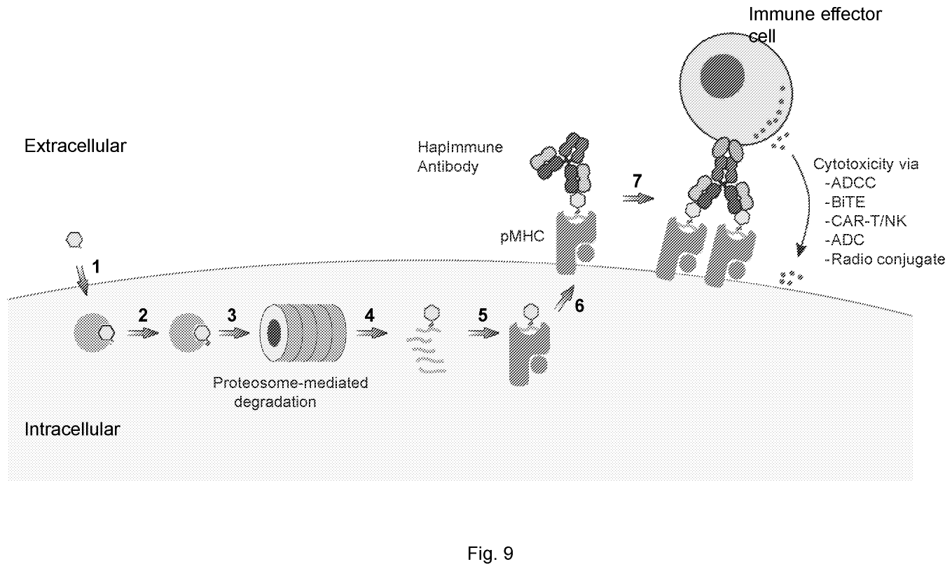

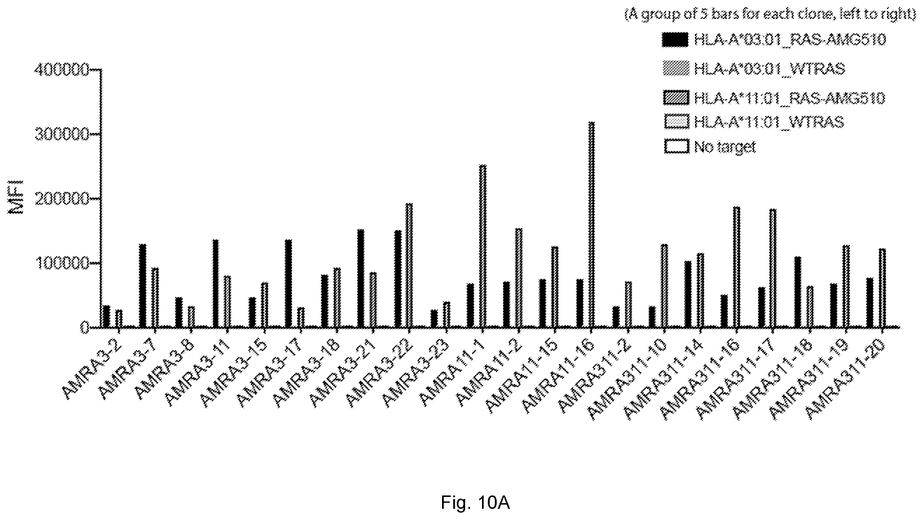

Data presented in this Example relates to FIGS. 9-15, 17, which provide the following information:

FIG. 9 provides a cartoon representation of a concept of the disclosure referred to as HapImmune™. The numbers 1-7 denote relevant steps. 1. A covalent inhibitor is administered, and it enters the cell harboring the target protein. 2. The inhibitor binds the target and forms a covalent bond with the target. 3 and 4. As a part of natural protein turnover (or induced protein degradation in the case of a PROTAC), the target-drug conjugate is degraded by the proteosome system. As a result, peptides with the conjugated drug are produced. 5. A peptide conjugate is incorporated into a compatible MHC molecule. 6. The MHC/peptide-drug conjugate complex translocates to the surface of the cell. A HapImmune™ antibody recognizes the complex. 7. The surface bound antibody recruits an immune effector cell, such as an NK cell, which in turn initiates cell killing activities. Multiple modalities are envisioned for effecting cell killing activities, including ADCC, ADCP, CDC, BITE, CAR-T, CAR-NK, ADC, and radioisotope conjugate, but they are not explicitly depicted here.

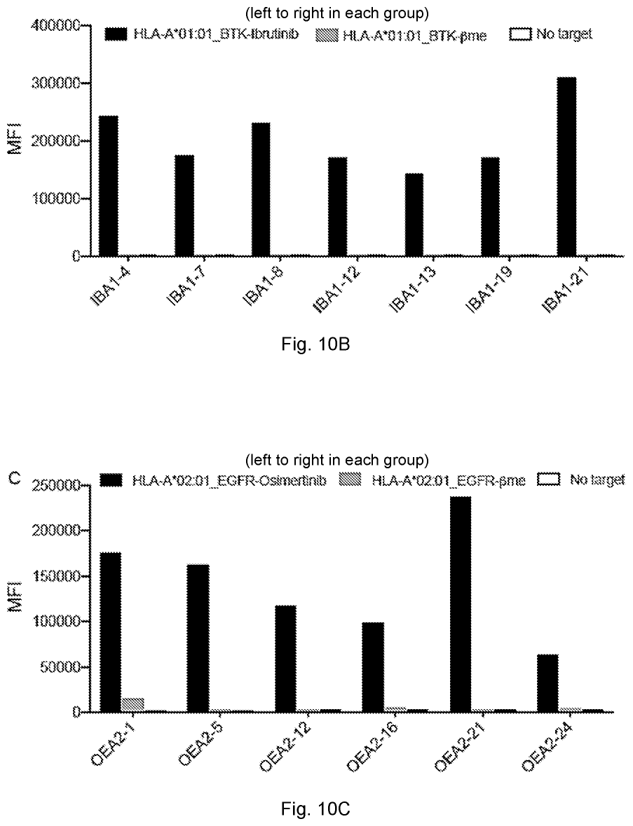

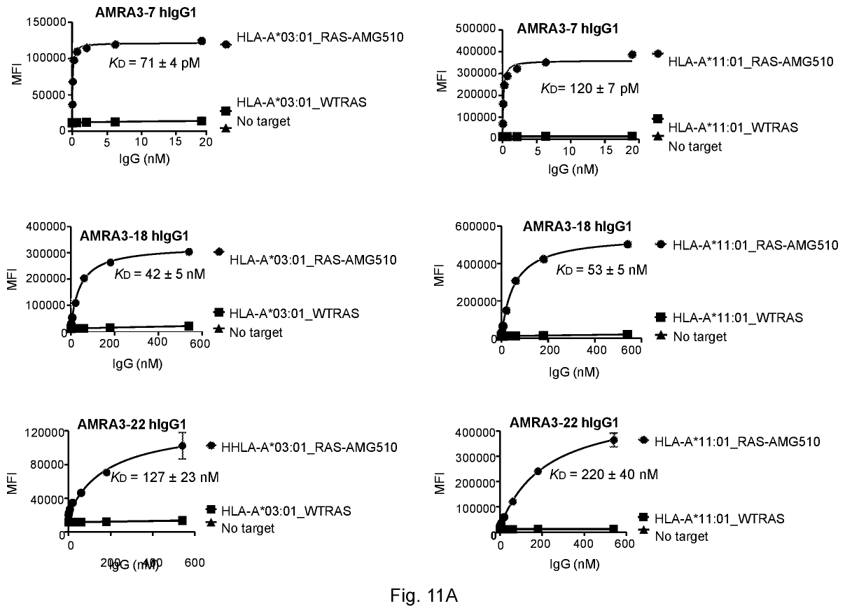

FIG. 10 shows data from development of antibodies that bind MHC/peptide-drug conjugate complexes. (A) Multiplex bead-binding assay (MBBA)⁽¹⁾ of phages displaying different antibody clones. For each phage clone, binding to a total of five antigens presented on beads was tested: HLA-A*03:01 in complex with the KRAS(G12C) peptide conjugated with AMG-510 (denoted as HLA-A*03:01_RAS-AMG510 in the figure); HLA-A*03:01 in complex with the KRAS (wild type) peptide (denoted as HLA-A*03:01_WTRAS); HLA-A*11:01 in complex with the KRAS(G12C) peptide conjugated with AMG-510 (denoted as HLA-A*11:01 RAS-AMG510); HLA-A*11:01 in complex with the KRAS (wild type) peptide (denoted as HLA-A*11:01_WTRAS); beads presenting no antigen (denoted as No target). (B) MBBA assay of phages displaying different antibody clones to: HLA-A*01:01 in complex with the BTK peptide conjugated with Ibrutinib (denoted as HLA-A*01:01 BTK-Ibrutinib in the figure); HLA-A*01:01 in complex with the BTK peptide conjugated with beta-mercaptoethanol (denoted as HLA-A*01:01_BTK-βme); beads presenting no antigen (denoted as No target). (C) MBBA assay of phages displaying different antibody clones to: HLA-A*02:01 in complex with the EGFR peptide conjugated with osimertinib (denoted as HLA-A*02:01_EGFR-osimertinib in the figure); HLA-A*02:01 in complex with the EGFR peptide conjugated with beta-mercaptoethanol (denoted as HLA-A*02:01_EGFR-βme); beads presenting no antigen (denoted as No target).

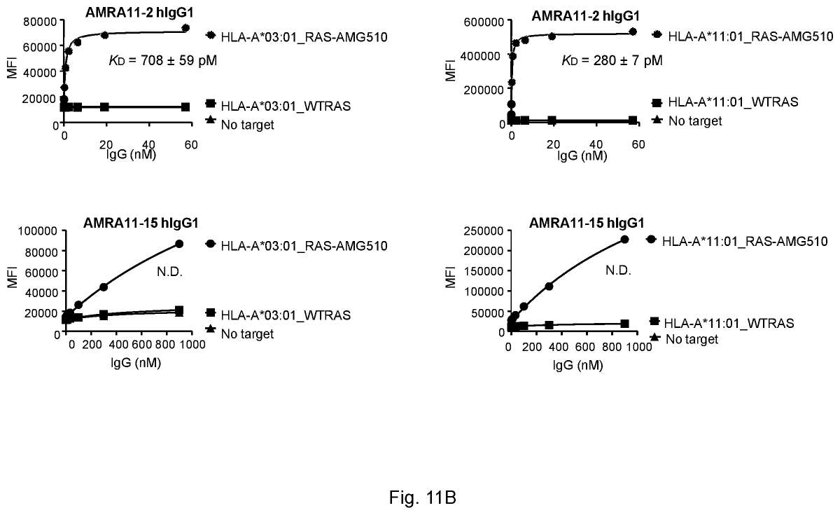

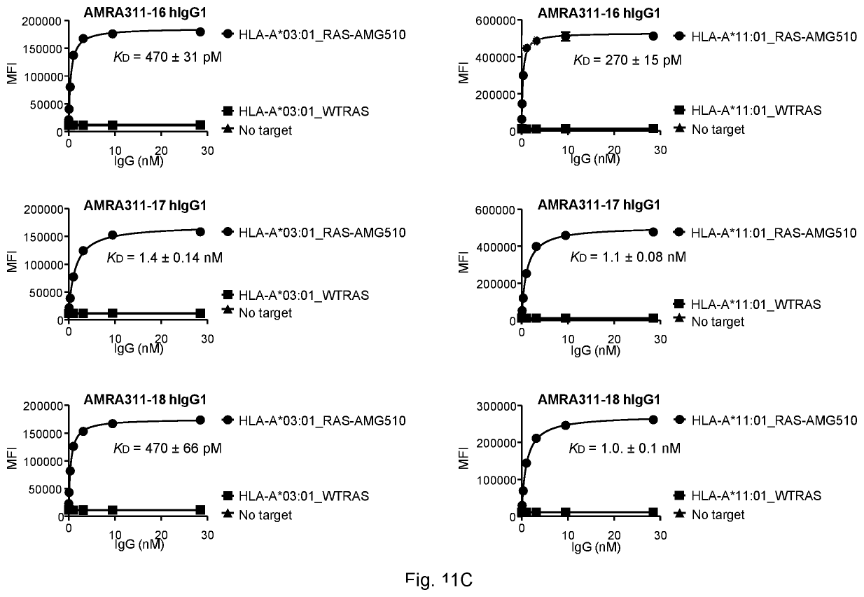

FIG. 11 shows results from binding titration using the multiplex bead-binding assay (MBBA) of purified antibodies targeted to the KRAS(G12C)-AMG510 conjugate. Clone names are shown over each graph. Antigen nomenclature is described in FIG. 10. The left column shows binding data with HLA-A*03:01 complexes, whereas the right column shows data with HLA-A*11:01 complexes. Apparent dissociation constant (K_{D}) values were determined using nonlinear least-squared fitting of a 1:1 binding function. The data for the wild-type RAS peptide complexes and for the no target were all close to the baseline and overlap, and thus their apparent K_{D} values were not determined. Data shown here are from triplicate measurements. Error bars are within the size of the symbols.

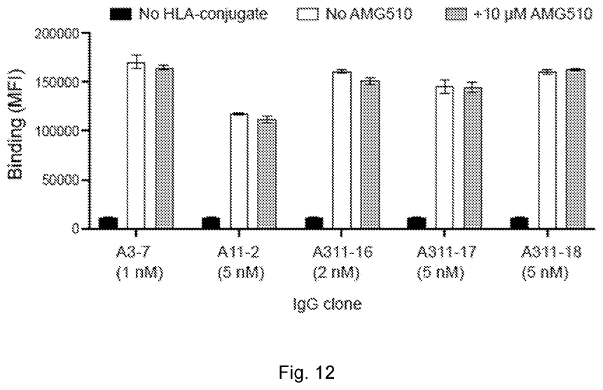

FIG. 12 demonstrates that binding of antibodies to the drug-peptide conjugate in complex with an MHC was not affected by the presence of the free drug. MBBA binding signals of select “AMR” series antibodies to HLA-A*03:01 in complex with the KRAS(G12C) peptide conjugated with AMG-510 in the absence (the white bars) and presence (the gray bars) of 10 μM free AMG-510 are shown. The antibody concentrations were adjusted to give sub-saturating signals and are shown in parentheses. Data shown here are from triplicate measurements.

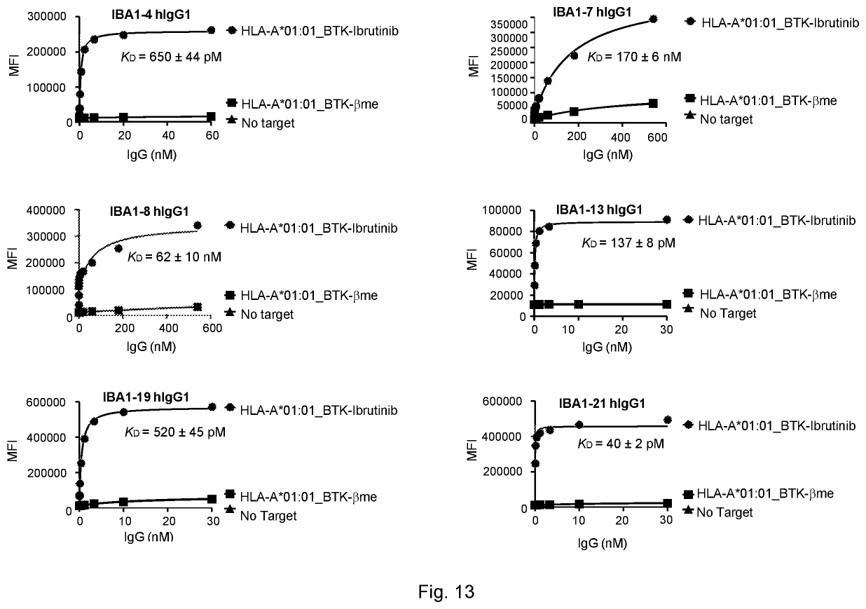

FIG. 13 shows results from binding titrations using the multiplex bead-binding assay (MBBA) of purified antibodies targeted to the BTK-Ibrutinib conjugate. Clone names are shown over each graph. Antigen nomenclature is described in FIG. 10. Apparent dissociation constant (K_{D}) values were determined using nonlinear least-squared fitting of a 1:1 binding function. The data for the beta-mercaptoethanol-conjugated peptide in complex with HLA-A*01:01 and for the no target were all close to the baseline and overlap, and thus their apparent K_{D} values were not determined. Data shown here are from triplicate measurements. Error bars are within the size of the symbols.

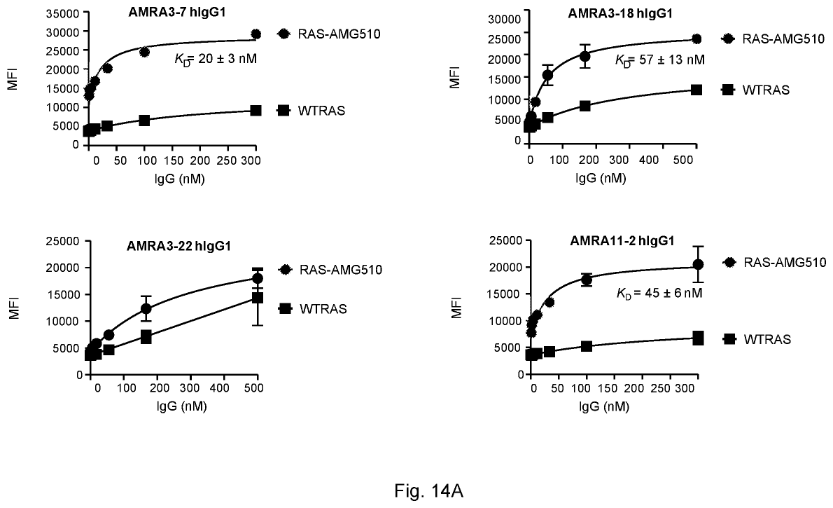

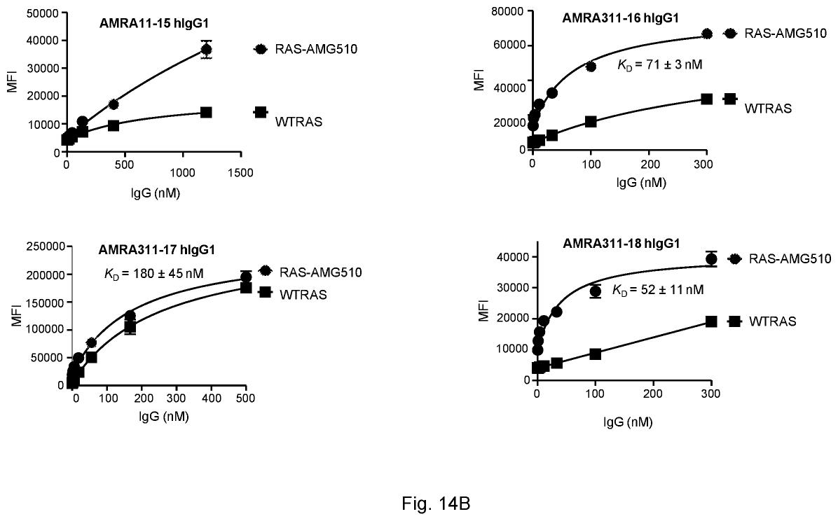

FIG. 14 shows results from binding titrations of purified antibodies to the KRAS(G12C)-AMG510 conjugate presented by endogenous MHC molecules on the cell surface. Raji cells were first incubated with the KRAS(G12C)-AMG510 conjugate or the KRAS (wild type) peptide, and excess conjugate and peptide were washed away. Surface-bound antibody levels detected using a fluorescently labeled secondary antibody are shown as a function of IgG concentration used for staining. Apparent dissociation constant (K_{D}) values were determined using nonlinear least-squared fitting of a 1:1 binding function. Data shown here are from triplicate measurements.

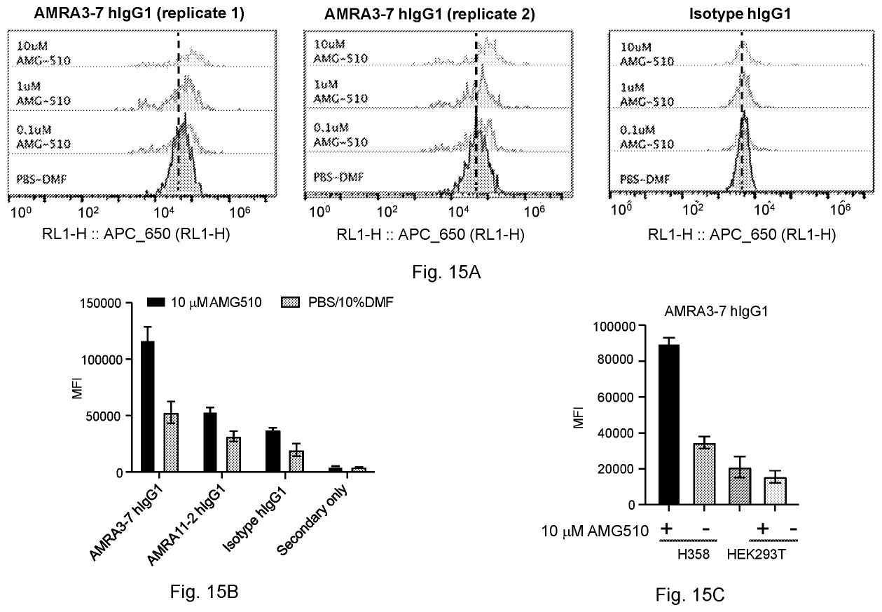

FIG. 15 shows results from an antibody binding to a KRAS(G12C)-expressing cell line pretreated with AMG510. The non-small cell lung cancer cell line H358 was incubated with AMG-510 for 2 days and then stained with antibodies targeting the KRAS(G12C) peptide-AMG510 conjugate or an isotype control, followed by detection with a secondary antibody. (A) Flow cytometry histograms. (B) Quantification of the median fluorescence intensity of H358 cells treated with or without AMG510. The antibodies used are indicated along the horizontal axis. (C) Quantification of the median fluorescence intensity of H358 cells and HEK293T cells (a negative control) treated with or without AMG-510 and stained with the AMRA3-7 antibody.

FIG. 16 is related to Example 2 and shows binding of P2AMR-1 IgG to cells preincubated with the KRAS(G12C) peptide-AMG510 conjugate, KRAS (wild type) peptide, or no peptide. The antibody was precomplexed with a dye-labeled secondary antibody in order to enhance the effective binding (avidity). The antibody bound to the Raji cells that express HLA-A*03:01 when the cells were incubated with the conjugate.

FIG. 17 shows results from binding of purified antibodies in the IgG format to the indicated drug-peptide/MHC complexes as measured using the multiplex bead binding assay (MBBA).

FIGS. 18 and 19 are discussed in Example 4.

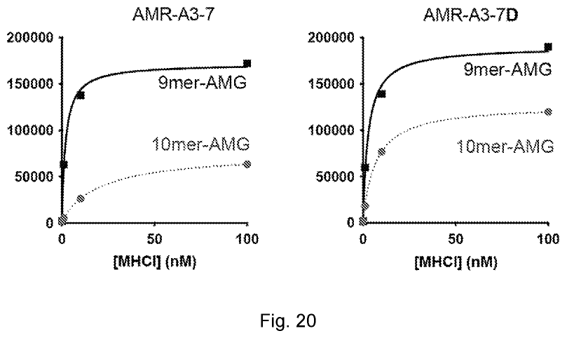

FIG. 20 shows results from binding titration curves of AMR-A3-7 and AMR-A3-7D displayed on the yeast surface. Binding to HLA-A*03:01 presenting AMG-510 conjugated to the Cys residue in the 9mer and 10mer RAS(G12C) peptides, VVGACGVGK and VVVGACGVGK, respectively, is shown.

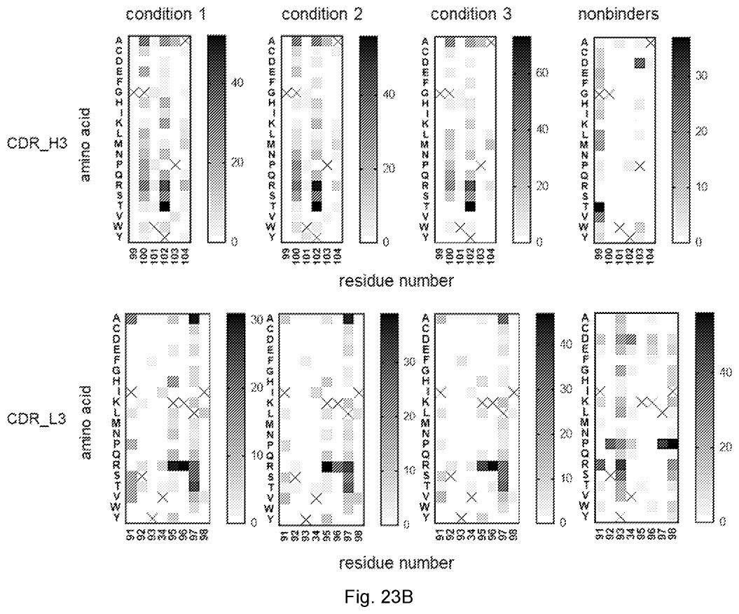

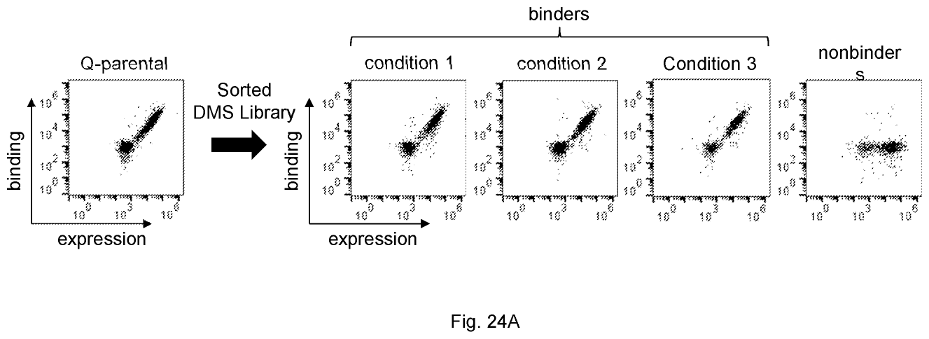

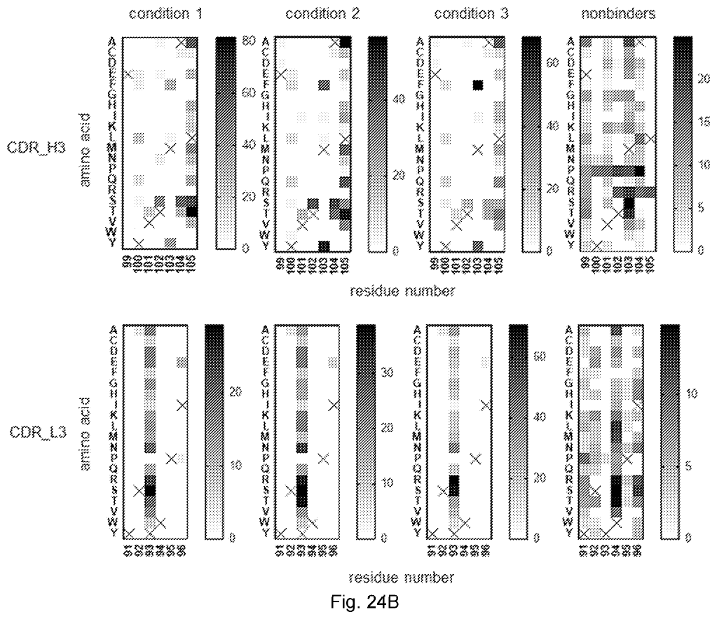

FIGS. 23 and 24 show results from deep mutational scanning of CDR-L3 and CDR-H3 of AMR-A3-7D and OEA2-5, respectively.

Specific and non-limiting examples of antibody sequences that bind in an MHC-drug conjugate-specific manner are as follows:

Exemplary Antibody Clones Binding to KRAS(G12C)-AMG510 conjugate presented on HLA-A*03:01 and HLA-A*11:01. CDR residues (Kabat scheme) in bold.

AMRA3-2
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSGWSYPITFGQGTKVEI
KRTV
V_{H}:
EVQLVESGGGLVQPGGSLRLSCAASGFTFYSSYIHWVRQAPGKGLEWVASIS
PYYGSTSYADSVKGRFTISADTSKNTAYLQMNSLRAEDTAVYYCARSSYYA
LDYWGQGTLVTVSS
AMRA3-7
V_{L}:
DIQMTQSPSSLSASVGDRVTITCRASQSVSSAVAWYQQKPGKAPKLLIYSASSL
YSGVPSRFSGSRSGTDFTLTISSLQPEDFATYYCQQISYVYSLITFGQGTKVEI
KRTV)
V_{H}:
EVQLVESGGGLVQPGGSLRLSCAASGFTFSSYSIHWVRQAPGKGLEWVASIY
SSYGYTSYADSVKGRFTISADTSKNTAYLQMNSLRAEDTAVYYCARGGWY
PAMDYWGQGTLVTVSS
AMRA3-7KK
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQISYVKKLITFGQGTKV
EIKRTV
V_{H}:
EVQLVESGGGLVQPGGSLRLSCAASGFTFSSYSIHWVRQAPGKGLEWVASIY
SSYGYTSYADSVKGRFTISADTSKNTAYLQMNSLRAEDTAVYYCARGGWY
PAMDYWGQGTLVTVSS
AMRA3-7D
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQISYVKKLITFGQGTKV
EIKRTV
V_{H}:
EVQLVESGGGLVQPGGSLRLSCAASGFTFSDYSIHWVRQAPGKGLEWVASIS
SSSGSTSYADSVKGRFTISADTSKNTAYLQMNSLRAEDTAVYYCARGGWYP
AMDYWGQGTLVTVSS
AMRA3-8
V_{L}:
DIQMTQSPSSLSASVGDRVTITCRASQSVSSAVAWYQQKPGKAPKLLIYSASS
LYSGVPSRFSGSRSGTDFTLTISSLQPEDLATYYCQQYQYGYNLITFGQGTKV
EIKRTV
V_{H}:
EVQLVESGGGLVQPGGSLRLSCAASGFTISYSSIHWVRQAPGKGLEWVASIYS
YSGSTSYADSVKGRFTISADTSKNTAYLQMNSLRAEDTAVYYCARYSYGWV
GPGWRAIDYWGQGTLVTVSS
AMRA3-11
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSVYKLLTFGQGTKV
EIKRTV
V_{H}:
EVQLVESGGGLVQPGGSLRLSCAASGFTVYYSSIHWVRQAPGKGLEWVASIS
SSYSYTSYADSVKGRFTISADTSKNTAYLQMNSLRAEDTALYYCARGGPGW
YRAMDYWGQGTLVTVSS
AMRA3-15
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTFSSSSIHWVRQAPGKGLEWVASIS
SSSGSTSYADSVKGRFTISADTSKNTAYLQMNSLRAEDTAVYYCARGYFYYG
WWAMAFDYWGQGTLVTVSS
AMRA3-17
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSQWYEPLITFGQGTKV
EIKRTV
V_{H}:
EVQLVESGGGLVQPGGSLRLSCAASGFTIYSSYIHWVRQAPGKGLEWVASISS
SSGSTSYADSVKGRFTISADTSKNTAYLQMNSLRAEDTAVYYCARSYSYMS
QWGWYQYSGMDYWGQGTLVTVSS
AMRA3-18
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQGSYTYRLITFGQGTKV
EIKRTV
V_{H}:
EVQLVESGGGLVQPGGSLRLSCAASGFTVSYSSIHWVRQAPGKGLEWVASIS
SSSGYTSYADSVKGRFTISADTSKNTAYLQMNSLRAEDTAVYYCARYAWW
AHGLDYWGQGTLVTVSS
AMRA3-21
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQASYWYNLFTFGQGTKV
EIKRTV
V_{H}:
EVQLVESGGGLVQPGGSLRLSCAASGFTISSYSIHWVRQAPGKGLEWVASIYS
SYGYTSYADSVKGRFTISADTSKNTAYLQMNSLRAEDTAVYYCARQYSMHF
PWGYGMDYWGQGTLVTVSS
AMRA3-22
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSDMPPITFGQGTKVEIK
RTV
V_{H}:
EVQLVESGGGLVQPGGSLRLSCAASGFTFYSSSIHWVRQAPGKGLEWVAYIY
SSSGYTSYADSVKGRFTISADTSKNTAYLQMNSLRAEDTAVYYCARPVNYY
YQGALDYWGQGTLVTVSS
AMRA3-23
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYYVFPITFGQGTKVEIK
RTV
V_{H}:
EVQLVESGGGLVQPGGSLRLSCAASGFTVYSSSIHWVRQAPGKGLEWVASIS
PSSGYTYYADSVKGRFTISADTSKNTAYLQMNSLRAEDTAVYYCARYHYMF
EYDKGESKWGYYGFDYWGQGTLVTVSS
AMRA11-1
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSQYFPITFGQGTKVEIK
RTV
V_{H}:
EVQLVESGGGLVQPGGSLRLSCAASGFTISYSSIHWVRQAPGKGLEWVASIYS
YYGYTSYADSVKGRFTISADTSKNTAYLQMNSLRAEDTAVYYCARNSWSW
YSGVGMDYWGQGTLVTVSS
AMRA11-2
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTISSSSIHWVRQAPGKGLEWVASISS
YSSSTYYADSVKGRFTISADTSKNTAYLQMNSLRAEDTAVYYCARYPYGWG
WGGSGLDYWGQGTLVTVSS
AMRA11-15
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQFDFQYLITFGQGTKVEI
KRTV
V_{H}:
EVQLVESGGGLVQPGGSLRLSCAASGFTVYYSSIHWVRQAPGKGLEWVASIY
SYYGSTYYADSVKGRFTISADTSKNTAYLQMNSLRAEDTAVYYCARGEKW
ALDYWGQGTLVTVSS
AMRA11-16
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYMYYQPLITFGQGTKV
EIKRTV
V_{H}:
EVQLVESGGGLVQPGGSLRLSCAASGFTVYYSSIHWVRQAPGKGLEWVASIS
SSSGSTSYADSVKGRFTISADTSKNTAYLQMNSLRAEDTAVYYCAREPYNYN
WYGMDYWGQGTLVTVSS
AMRA311-2
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSLWWPITFGQGTKVEI
KRTV
V_{H}:
EVQLVESGGGLVQPGGSLRLSCAASGFTVSSSSIHWVRQAPGKGLEWVASIY
SYSGYTSYADSVKGRFTISADTSKNTAYLQMNSLRAEDTAVYYCARHGSYG
SWWALDYWGQGTLVTVSS
AMRA311-10
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYFYFPITFGQGTKVEIK
RTV
V_{H}:
EVQLVESGGGLVQPGGSLRLSCAASGFTFYSSSIHWVRQAPGKGLEWVASISS
YYGSTSYADSVKGRFTISADTSKNTAYLQMNSLRAEDTAVYYCARASYYSG
YGSSYPYYMGLDYWGQGTLVTVSS
AMRA311-14
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQGSYRNPLLTFGQGTKV
EIKRTV
V_{H}:
EVQLVESGGGLVQPGGSLRLSCAASGFTFSSYSIHWVRQAPGKGLEWVASISS
SSGYTSYADSVKGRFTISADTSKNTAYLQMNSLRAEDTAVYYCARMNWSH
YAMDYWGQGTLVTVSS
AMRA311-16
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTISSSSIHWVRQAPGKGLEWVAYISS
YSGYTYYADSVKGRFTISADTSKNTAYLQMNSLRAEDTAVYYCARYWYGH
YHSYFGLDYWGQGTLVTVSS
AMRA311-17
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEIKR
TV
V_{H}:
EVQLVESGGGLVQPGGSLRLSCAASGFTISSSSIHWVRQAPGKGLEWVASISS
YSGSTYYADSVKGRFTISADTSKNTAYLQMNSLRAEDTAVYYCARYPYGSH
VYTGLDYWGQGTLVTVSS
AMRA311-18
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQWNWADYLVTFGQGTK
VEIKRTV
V_{H}:
EVQLVESGGGLVQPGGSLRLSCAASGFTISSSSIHWVRQAPGKGLEWVASIYS
SSGSTSYADSVKGRFTISADTSKNTAYLQMNSLRAEDTAVYYCARVYSSRY
WGWGVAFDYWGQGTLVTVSS
AMRA311-19
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYYWYSLITFGQGTKVE
IKRTV
V_{H}:
EVQLVESGGGLVQPGGSLRLSCAASGFTVYSSSIHWVRQAPGKGLEWVAYIY
SSSGYTSYADSVKGRFTISADTSKNTAYLQMNSLRAEDTAVYYCARRSFPQ
WYNGSYTPWPAMDYWGQGTLVTVSS
AMRA311-20
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYMWWPVTFGQGTKVE
IKRTV
V_{H}:
EVQLVESGGGLVQPGGSLRLSCAASGFTVSSSSIHWVRQAPGKGLEWVASIY
SYSSYTSYADSVKGRFTISADTSKNTAYLQMNSLRAEDTAVYYCARPFYWG
ERYALDYWGQGTLVTVSS

Antibody clones that bind preferentially to KRAS(G12C)-AMG510 conjugate presented on HLA-A*03:01 relative to the same conjugate presented on HLA-A*11:01.

CDR residues (Kabat scheme) in bold.

AMRA3-5
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSYSTLVTFGQGTKVEI
KRTV
V_{H}:
EVQLVESGGGLVQPGGSLRLSCAASGFTFYSSSIHWVRQAPGKGLEWVASIY
SSYGSTSYADSVKGRFTISADTSKNTAYLQMNSLRAEDTAVYYCARIYGWS
YQGWAGMDYWGQGTLVTVSS
AMRA3-6
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTISYSSIHWVRQAPGKGLEWVASIYP
YYGSTSYADSVKGRFTISADTSKNTAYLQMNSLRAEDTAVYYCARGGDYY
WGWYWVAMDYWGQGTLVTVSS
AMRA3-9
V_{L}:
DIQMTQSPSSLSASVGDRVTITCRASQSVSSAVAWYQQKPGKAPKLLIYSASS
LYSGVPSRFSGSRSGTDFTLTIXSLQPEDFATYYCQKSSSSLITFGQGTKVEIK
RTV
V_{H}:
EVQLVESGGGLVQPGGSLRLSCAASGFTFSSSYIHWVRQAPGKGLEWVASISS
SYGSTSYADSVKGRFTISADTSKNTAYLQMNSLRAEDTAVYYCARMYYYTY
PGMDYWGQGTLVTVSS
AMRA3-10
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQKGSSYLLTFGQGTKVEIK
RTV
V_{H}:
EVQLVESGGGLVQPGGSLRLSCAASGFTIYSYSIHWVRQAPGKGLEWVASISP
SSGYTSYADSVKGRFTISADTSKNTAYLQMNSLRAEDTAVYYCARYHYGG
WSHYMSGMDYWGQGTLVTVSS
AMRA3-13
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQNYYYHKLITFGQGTKV
EIKRTV
V_{H}:
EVQLVESGGGLVQPGGSLRLSCAASGFTFSYSSIHWVRQAPGKGLEWVASISS
SYGYTSYADSVKGRFTISADTSKNTAYLQMNSLRAEDTAVYYCARGRYGG
MDYWGQGTLVTVSS
AMRA3-19
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQLSYVYKLITFGQGTKV
EIKRTV
V_{H}:
EVQLVESGGGLVQPGGSLRLSCAASGFTFYSSSIHWVRQAPGKGLEWVASISS
SYGYTSYADSVKGRFTISADTSKNTAYLQMNSLRAEDTAVYYCARGWYKA
MDYWGQGTLVTVSS
AMRA3-24
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEIKR
TV
V_{H}:
EVQLVESGGGLVQPGGSLRLSCAASGFTFSYSSIHWVRQAPGKGLEWVASISS
SYGSTYYADSVKGRFTISADTSKNTAYLQMNSLRAEDTAVYYCARMYYYY
YPGIDYWGQGTLVTVSS

Antibody clones that bind preferentially to KRAS(G12C)-AMG510 conjugate presented on HLA-A*11:01 relative to KRAS(G12C)-AMG510 conjugate presented on HLA-A*03:01.

AMRA11-3
V_{L}:
DIQMTQSPSSLSASVGDRVTITCRASQSVSSAVAWYQQKPGKAPKLLIYSASS
LYSGVPSRFSGSRSGTDFTLTISSLQPEDLATYYCQQYYYFPITFGQGTKVEIK
RTV
V_{H}:
EVQLVESGGGLVQPGGSLRLSCAASGFTFSSSSIHWVRQAPGKGLEWVASISP
YYGYTSYADSVKGRFTISADTSKNTAYLQMNSLRAEDTAVYYCARSPYYW
YQYFYGWGLDYWGQGTLVTVSS
AMRA11-4
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEIKR
TV
V_{H}:
EVQLVESGGGLVQPGGSLRLSCAASGFTFSYSSIHWVRQAPGKGLEWVASISS
SSGSTSYADSVKGRFTISADTSKNTAYLQMNSLRAEDTAVYYCARSPYWWN
YMSAMDYWGQGTLVTVSS
AMRA11-7
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQGWWWPFTFGQGTKVE
IKRTV
V_{H}:
EVQLVESGGGLVQPGGSLRLSCAASGFTVSSYSIHWVRQAPGKGLEWVASIS
PYYGYTSYADSVKGRFTISADTSKNTAYLQMNSLRAEDTAVYYCARWSWQ
YYSGHSSWGLDYWGQGTLVTVSS
AMRA11-8
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSWYFPLTFGQGTKVEI
KRTV
V_{H}:
EVQLVESGGGLVQPGGSLRLSCAASGFTVSSSSIHWVRQAPGKGLEWVASIY
SYYGYTSYADSVKGRFTISADTSKNTAYLQMNSLRAEDTAVYYCARWYNE
YYHDYYWDAMDYWGQGTLVTVSS
AMRA11-9
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEIKR
TV
V_{H}:
EVQLVESGGGLVQPGGSLRLSCAASGFTLYYSSIHWVRQAPGKGLEWVASIS
SSSGSTSYADSVKGRFTISADTSKNTAYLQMNSLRAEDTAVYYCARWMYW
WSFALDYWGQGTLVTVSS
AMRA11-10
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSYLWPITFGQGTKVEIK
RTV
V_{H}:
EVQLVESGGGLVQPGGSLRLSCAASGFTVSSSSIHWVRQAPGKGLEWVASIY
SYYGYTSYADSVKGRFTISADTSKNTAYLQMNSLRAEDTAVYYCARWQYH
YNYWYGMDYWGQGTLVTVSS
AMRA11-11
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYPMSLITFGQGTKVEIK
RTV
V_{H}:
EVQLVESGGGLVQPGGSLRLSCAASGFTVSYSSIHWVRQAPGKGLEWVASIS
PYSGYTSYADSVKGRFTISADTSKNTAYLQMNSLRAEDTAVYYCARGYDYY
AGLDYWGQGTLVTVSS
AMRA11-12
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYYYFPITFGQGTKVEIK
RTV
V_{H}:
EVQLVESGGGLVQPGGSLRLSCAASGFTFSYYSIHWVRQAPGKGLEWVASIS
PYYGYTSYADSVKGRFTISADTSKNTAYLQMNSLRAEDTAVYYCARWESEY
SGTYEDYWAGMDYWGQGTLVTVSS
AMRA11-13
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYMWWPITFGQGTKVEI
KRTV
V_{H}:
EVQLVESGGGLVQPGGSLRLSCAASGFTISYSSIHWVRQAPGKGLEWVASISS
SYSYTSYADSVKGRFTISADTSKNTAYLQMNSLRAEDTAVYYCARTGYWQ
GYLALDYWGQGTLVTVSS
AMRA11-14
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEIKR
TV
V_{H}:
EVQLVESGGGLVQPGGSLRLSCAASGFTISYSSIHWVRQAPGKGLEWVASISS
SSGSTSYADSVKGRFTISADTSKNTAYLQMNSLRAEDTAVYYCARTYYYYW
NSTPAMDYWGQGTLVTVSS
AMRA11-18
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSYGYPVTFGQGTKVEI
KRTV
V_{H}:
EVQLVESGGGLVQPGGSLRLSCAASGFTFSSSSIHWVRQAPGKGLEWVASISS
SYGYTSYADSVKGRFTISADTSKNTAYLQMNSLRAEDTAVYYCARWYNSS
WYYSNWWYKGFGMDYWGQGTLVTVSS
AMRA11-20
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYYSSLFTFGQGTKVEIK
RTV
V_{H}:
EVQLVESGGGLVQPGGSLRLSCAASGFTFYSSSIHWVRQAPGKGLEWVASISS
SYGYTSYADSVKGRFTISADTSKNTAYLQMNSLRAEDTAVYYCARTSYTYP
VYTYYGFDYWGQGTLVTVSS
AMRA11-22
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSWYYPLTFGQGTKVEI
KRTV
V_{H}:
EVQLVESGGGLVQPGGSLRLSCAASGFTLYSSSIHWVRQAPGKGLEWVASIS
SSYGYTSYADSVKGRFTISADTSKNTAYLQMNSLRAEDTAVYYCARYRYSS
WNRGAIDYWGQGTLVTVSS
AMRA11-24
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSYWWPLTFGQGTKVEI
KRTV
V_{H}:
EVQLVESGGGLVQPGGSLRLSCAASGFTVSSSSIHWVRQAPGKGLEWVASIY
SYYGYTSYADSVKGRFTISADTSKNTAYLQMNSLRAEDTAVYYCARWSKSP
WYYQGIDYWGQGTLVTVSS

Exemplary Antibody Clones Binding to BTK-Ibrutinib conjugate presented on HLA-A*01:01. CDR residues (Kabat scheme) in bold.

IBA1-4
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYHYWASLITFGQGTKV
EIKRTV
V_{H}:
EVQLVESGGGLVQPGGSLRLSCAASGFTVSSSSIHWVRQAPGKGLEWVASIY
SYSGSTYYADSVKGRFTISADTSKNTAYLQMNSLRAEDTAVYYCARQYSSS
YYVWPGMDYWGQGTLVTVSS
IBA1-7
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSYWWKSLVTFGQGTK
VEIKRTV
V_{H}:
EVQLVESGGGLVQPGGSLRLSCAASGFTLSSSSIHWVRQAPGKGLEWVASISS
YYGSTSYADSVKGRFTISADTSKNTAYLQMNSLRAEDTAVYYCARMHYSW
QEYYSYDWGMDYWGQGTLVTVSS
IBA1-8
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQPYYPLITFGQGTKVEIK
RTV
V_{H}:
EVQLVESGGGLVQPGGSLRLSCAASGFTISYSSIHWVRQAPGKGLEWVASIYP
SYGSTSYADSVKGRFTISADTSKNTAYLQMNSLRAEDTAVYYCARWQGYY
QPALDYWGQGTLVTVSS
IBA1-12
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSKYYYPITFGQGTKV
EIKRTV
V_{H}:
EVQLVESGGGLVQPGGSLRLSCAASGFTISSSSIHWVRQAPGKGLEWVASISP
YYGYTSYADSVKGRFTISADTSKNTAYLQMNSLRAEDTAVYYCARWGYG
WYWYGLDYWGQGTLVTVSS
IBA1-13
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQGHDMNPVTFGQGTKV
EIKRTV
V_{H}:
EVQLVESGGGLVQPGGSLRLSCAASGFTLSSSSIHWVRQAPGKGLEWVASIY
SSYGYTYYADSVKGRFTISADTSKNTAYLQMNSLRAEDTAVYYCARYYYY
WYGGMDYWGQGTLVTVSS
IBA1-19
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSWMSDSLITFGQGTKV
EIKRTV
V_{H}:
EVQLVESGGGLVQPGGSLRLSCAASGFTFSYSSIHWVRQAPGKGLEWVASIY
PSSGYTSYADSVKGRFTISADTSKNTAYLQMNSLRAEDTAVYYCARGWWY
WMAWDYAMDYWGQGTLVTVSS
IBA1-21
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQMQYSGWLITFGQGTK
VEIKRTV
V_{H}:
EVQLVESGGGLVQPGGSLRLSCAASGFTISSSSIHWVRQAPGKGLEWVASISS
YYGYTSYADSVKGRFTISADTSKNTAYLQMNSLRAEDTAVYYCARYYSYSS
GYGYYDYFDWGAMDYWGQGTLVTVSS

Exemplary Antibody Clones binding to EGFR-osimertinib conjugate presented on HLA-A*02:01. CDR residues (Kabat scheme) in bold.

OEA2-1
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTFSSSSIHWVRQAPGKGLEWVASISS
SSGSTSYADSVKGRFTISADTSKNTAYLQMNSLRAEDTAVYYCARYYGYVW
GGYWGWWYSKALDYWGQGTLVTVSS
OEA2-5
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYSYWPITFGQGTKVEIK
RTV
V_{H}:
EVQLVESGGGLVQPGGSLRLSCAASGFTISSSYIHWVRQAPGKGLEWVAYISP
SYGSTSYADSVKGRFTISADTSKNTAYLQMNSLRAEDTAVYYCAREYVTMA
LDYWGQGTLVTVSS
OEA2-12
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQYDWNYYLVTFGQGTK
VEIKRTV
V_{H}:
EVQLVESGGGLVQPGGSLRLSCAASGFTIYSSSIHWVRQAPGKGLEWVASISS
YYGYTSYADSVKGRFTISADTSKNTAYLQMNSLRAEDTAVYYCARYQYYG
SLYYSQQWAMDYWGQGTLVTVSS
OEA2-16
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTFSSSSIHWVRQAPGKGLEWVASISS
SSGSTSYADSVKGRFTISADTSKNTAYLQMNSLRAEDTAVYYCARSPSSPYF
MSWGWYWQYGIDYWGQGTLVTVSS
OEA2-21
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSWGGLVTFGQGTKVE
IKRTV
V_{H}:
EVQLVESGGGLVQPGGSLRLSCAASGFTFSSSYIHWVRQAPGKGLEWVASIS
PSYGYTSYADSVKGRFTISADTSKNTAYLQMNSLRAEDTAVYYCARDMYE
WWHWAIDYWGQGTLVTVSS
OEA2-24
V_{L}:
DIQMTQSPSSLSASVGDRVTITCRASQSVSSAVAWYQQKPGKAPKLLIYSASS
LYSGVPSRFSGSRSGTDFTLTISSLQPEDFATYYCQQSSSSLITFGQGTKVEI
KRTV
V_{H}:
EVQLVESGGGLVQPGGSLRLSCAASGFTFSSSSIHWVRQAPGKGLEWVASISS
SSGSTSYADSVKGRFTISADTSKNTAYLQMNSLRAEDTAVYYCARYGHYLY
YWGWGWYWSAALDYWGQGTLVTVSS

### References Related to Example 3

1. Miller K R, Koide A, Leung B, Fitzsimmons J, Yoder B, Yuan H, Jay M, Sidhu S S, Koide S, Collins E J. T cell receptor-like recognition of tumor in vivo by synthetic antibody fragment. PloS one. 2012; 7 (8): e43746. Epub 2012 Aug. 24. doi: 10.1371/journal.pone.0043746. PubMed PMID: 22916301; PMCID: 3423377.
2. Hattori T, Koide A, Panchenko T, Romero L A, Teng K W, Corrado A D, Koide S. Multiplex bead binding assays using off-the-shelf components and common flow cytometers. J Immunol Methods. 2020:112952. Epub 2020 Dec. 29. doi:10.1016/j.jim.2020.112952. PubMed PMID: 33358997.

### EXAMPLE 4

This Example demonstrates single-chain Diabody (scDb) formats of HapImmune™ antibodies and their effectiveness in cell killing. Data from non-limiting embodiments are presented in FIGS. 18 and 19. The results are summarized as in the brief descriptions of FIGS. 18 and 19.

To obtain the results for FIG. 18, Raji cells and T2 cells (ATCC) were cultured in RPMI supplemented with 10% fetal bovine serum (FBS) and penicillin/streptomycin. The cytotoxic effect of scDbs was measured by following the protocol published previously (ref: PMID 26813960). Briefly, Raji cells or T2 cells were stained with carboxyfluorescein succinimidyl ester (CSFE, ThermoFisher, 65-0850-84), then incubated with the final 10 μM KRAS(G12C)-AMG510 conjugate or 1 μM EGFR-osimertinib in the presence of 10 μg/mL human beta-2 microglobulin for 4 hr. The cells were harvested using centrifugation and washed in media to remove the unbound conjugate and peptide. Peptide-drug-pulsed cells were then co-cultured with human T-cells (E:T=3:1) in the presence of single-chain Diabodies (scDbs) for 19-21 hr. After incubation, cells were harvested and washed with PBS, then stained with Fixable Viability Dye eFluor660 (ThermoFisher, 65-0864-14). After washing cells, the cells were analyzed on iQue screener (Sartorius).

To obtain the results for FIG. 19, lung cancer cell lines were cultured in RPMI supplemented with 10% fetal bovine serum (FBS) and penicillin/streptomycin. For cytotoxicity assays, 1×10⁴ cells/well were seeded in 96-well flat bottom plates and incubated at 37° C., 5% CO₂ for 24 hours. Media were replaced with fresh media supplemented with 100 nM AMG-510 or DMSO, then the cells were incubated for 24 hours at 37° C. After incubation, cells were co-cultured with human T cells (E:T=5:1) and AMRA3-7_UCHT1 scDb in the presence of 100 IU/mL IL-2 for 24 hr at 37° C. Cell viability was assessed by using PrestoBlue™ Cell Viability Reagent (ThermoFisher, A13261). Cytotoxicity was calculated by taking the fluorescent signal of a given well, subtracting the fluorescent signal from the wells that contain only T-cells, and normalizing to the fluorescent signal from the wells without scDb.

### Exemplary Single-Chain Diabody Clones Targeting Both HLA-A*03:01 RAS-AMG510 and HLA-A*11:01 RAS-AMG510

The italicized sequences represent AviTag and HisTag, respectively.

AMRA3-7_UCHT1_scDb
DIVRSDIQMTQSPSSLSASVGDRVTITCRASQSVSSAVAWYQQKP
GKAPKLLIYSASSLYSGVPSRFSGSRSGTDFTLTISSLQPEDFAT
YYCQQISYVYSLITFGQGTKVEIKGGGGSEVQLQQSGPELVKPGA
SMKISCKASGYSFTGYTMNWVKQSHGKNLEWMGLINPYKGVSTYN
QKFKDKATLTVDKSSSTAYMELLSLTSEDSAVYYCARSGYYGDSD
WYFDVWGQGTTLTVSSGGGGSGGGGSGGGGSDIQMTQTTSSLSAS
LGDRVTISCRASQDIRNYLNWYQQKPDGTVKLLIYYTSRLHSGVP
SKFSGSGSGTDYSLTISNLEQEDIATYFCQQGNTLPWTFAGGTKL
EIKGGGGSEVQLVESGGGLVQPGGSLRLSCAASGFTFSSYSIHWV
RQAPGKGLEWVASIYSSYGYTSYADSVKGRFTISADTSKNTAYLQ
MNSLRAEDTAVYYCARGGWYPAMDYWGQGTLVTVSSLEGGGGLND
IFEAQKIEWHESRHHHHHH
AMRA311-16_UCHT1_scDb
DIVRSDIQMTQSPSSLSASVGDRVTITCRASQSVSSAVAWYQQKP
GKAPKLLIYSASSLYSGVPSRFSGSRSGTDFTLTISSLQPEDFAT
YYCQQSSSSLITFGQGTKVEIKGGGGSEVQLQQSGPELVKPGASM
KISCKASGYSFTGYTMNWVKQSHGKNLEWMGLINPYKGVSTYNQK
FKDKATLTVDKSSSTAYMELLSLTSEDSAVYYCARSGYYGDSDWY
FDVWGQGTTLTVSSGGGGSGGGGSGGGGSDIQMTQTTSSLSASLG
DRVTISCRASQDIRNYLNWYQQKPDGTVKLLIYYTSRLHSGVPSK
FSGSGSGTDYSLTISNLEQEDIATYFCQQGNTLPWTFAGGTKLEI
KGGGGSEVQLVESGGGLVQPGGSLRLSCAASGFTISSSSIHWVRQ
APGKGLEWVAYISSYSGYTYYADSVKGRFTISADTSKNTAYLQMN
SLRAEDTAVYYCARYWYGHYHSYFGLDYWGQGTLVTVSSLEGGGG
LNDIFEAQKIEWHESRHHHHHH

### Exemplary Single-Chain Diabody Clones Targeting HLA-A*02:01_EGFR-Osimertinib

The italicized sequences represent AviTag and HisTag, respectively.

QEA2-5_UCHT1_scDb
DIVRSDIQMTQSPSSLSASVGDRVTITCRASQSVSSAVAWYQQKP
GKAPKLLIYSASSLYSGVPSRFSGSRSGTDFTLTISSLQPEDFAT
YYCQQYSYWPITFGQGTKVEIKGGGGSEVQLQQSGPELVKPGASM
KISCKASGYSFTGYTMNWVKQSHGKNLEWMGLINPYKGVSTYNQK
FKDKATLTVDKSSSTAYMELLSLTSEDSAVYYCARSGYYGDSDWY
FDVWGQGTTLTVSSGGGGSGGGGSGGGGSDIQMTQTTSSLSASLG
DRVTISCRASQDIRNYLNWYQQKPDGTVKLLIYYTSRLHSGVPSK
FSGSGSGTDYSLTISNLEQEDIATYFCQQGNTLPWTFAGGTKLEI
KGGGGSEVQLVESGGGLVQPGGSLRLSCAASGFTISSSYIHWVRQ
APGKGLEWVAYISPSYGSTSYADSVKGRFTISADTSKNTAYLQMN
SLRAEDTAVYYCAREYVTMALDYWGQGTLVTVSSLEGGGGLNDIF
EAQKIEWHESRHHHHHH
QEA2-21_UCHT1_scDb
DIVRSDIQMTQSPSSLSASVGDRVTITCRASQSVSSAVAWYQQKP
GKAPKLLIYSASSLYSGVPSRFSGSRSGTDFTLTISSLQPEDFAT
YYCQQSSWGGLVTFGQGTKVEIKGGGGSEVQLQQSGPELVKPGAS
MKISCKASGYSFTGYTMNWVKQSHGKNLEWMGLINPYKGVSTYNQ
KFKDKATLTVDKSSSTAYMELLSLTSEDSAVYYCARSGYYGDSDW
YFDVWGQGTTLTVSSGGGGSGGGGSGGGGSDIQMTQTTSSLSASL
GDRVTISCRASQDIRNYLNWYQQKPDGTVKLLIYYTSRLHSGVPS
KFSGSGSGTDYSLTISNLEQEDIATYFCQQGNTLPWTFAGGTKLE
IKGGGGSEVQLVESGGGLVQPGGSLRLSCAASGFTFSSSYIHWVR
QAPGKGLEWVASISPSYGYTSYADSVKGRFTISADTSKNTAYLQM
NSLRAEDTAVYYCARDMYEWWHWAIDYWGQGTLVTVSSLEGGGGL
NDIFEAQKIEWHESRHHHHHH

In various embodiments, the binding partners described herein do not comprise the amino acid sequence LEGGGGLNDIFEAQKIEWHESRHHHHHH. In various embodiments, the binding partners described herein do not comprise an AviTag or a His tag. In various embodiments, the binding partners to be administered into a subject do not comprise the amino acid sequence LEGGGGLNDIFEAQKIEWHESRHHHHHH. In various embodiments, the binding partners to be administered into a subject do not comprise an AviTag or a His tag.

### EXAMPLE 5

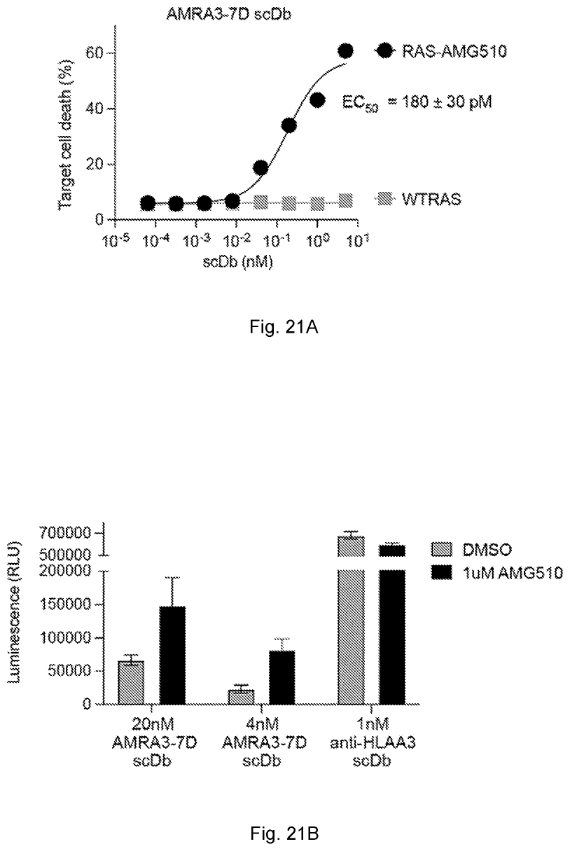

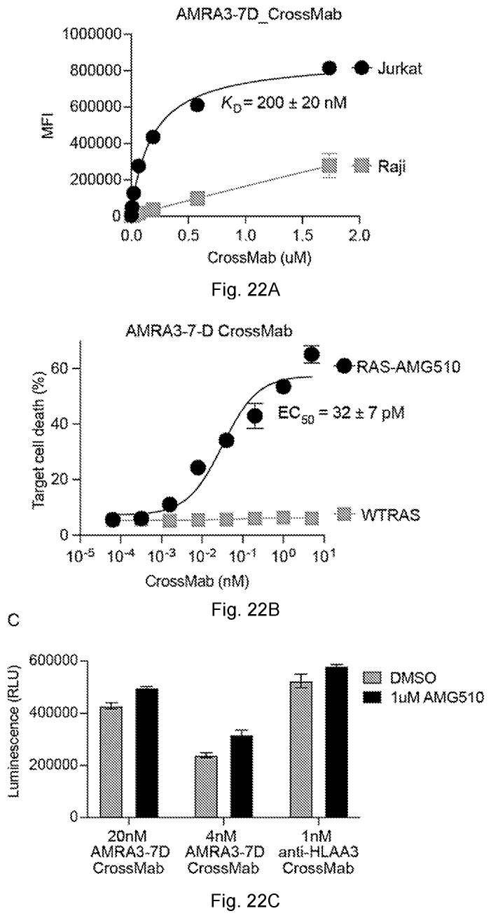

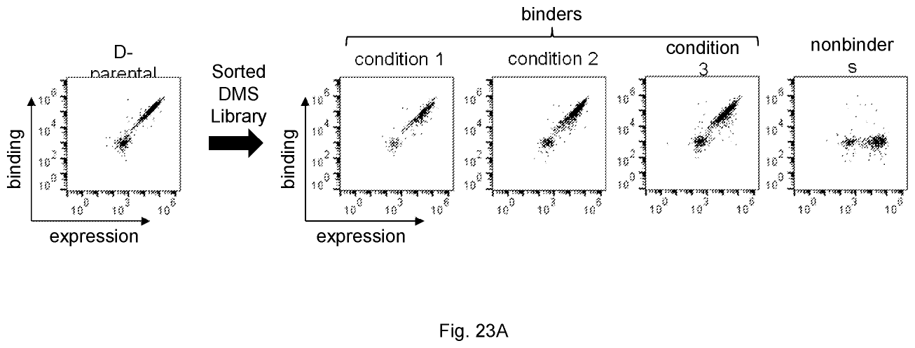

This Example demonstrates scDb and 2+1 CrossMab antibodies constructed with the AMRA3-7 clone and their effectiveness in cell killing. Data from non-limiting embodiments are presented in FIGS. 21-22.

To obtain the results for FIG. 21 (A) and FIG. 22 (B), Raji cells (ATCC) were cultured in RPMI supplemented with 10% fetal bovine serum (FBS) and penicillin/streptomycin. The cytotoxic effects of scDb and CrossMab were measured by following the protocol published previously (ref: PMID 26813960). Briefly, Raji cells were stained with carboxyfluorescein succinimidyl ester (CSFE, ThermoFisher, 65-0850-84), then incubated with 10 μM KRAS(G12C)-AMG510 conjugate or 10 μM KRAS (WT) peptide (final concentrations) in the presence of 10 μg/mL human beta-2 microglobulin for 4 hr. The cells were harvested by centrifugation and washed in media to remove the unbound conjugate and peptide. Peptide-drug-pulsed cells were then co-cultured with human T-cells (E:T=3:1) in the presence of scDb or CrossMab for 19 hr. After incubation, cells were harvested and washed with PBS, then stained with Fixable Viability Dye eFluor660 (ThermoFisher, 65-0864-14). After washing again, cells were analyzed on iQue screener (Satorius).

To obtain the results for FIGS. 21 (B) and 22 (C), H2122 cells (ATCC) were cultured in RPMI supplemented with 10% fetal bovine serum (FBS) and penicillin/streptomycin. For cytotoxicity assays, 5×10³ cells/well were seeded in 96-well flat bottom plates in the presence of 1 mM AMG-510 or DMSO and 5 μg/mL human beta-2 microglobulin, and then were incubated at 37° C., 5% CO₂ for 48 hours. After incubation, cells were co-cultured with human T cells (E:T=10:1) and AMRA3-7D scDb or CrossMab in the presence of 10 ng/ml IL7 and IL15 for 24 hr at 37° C. Dead cells were measured by using CytoTox-Glo cytotoxic assay (Promega, G9290). The luminescent signal of a given well was calculated by subtracting the signal from wells that contain H2122 and T-cells without scDb or CrossMab constructs.

To obtain the results for FIG. 22 (A), Jurkat and Raji cells (ATCC) were cultured in RPMI supplemented with 10% fetal bovine serum (FBS) and penicillin/streptomycin. Cells were washed twice with PBS, then incubated with AMRA3-7D CrossMab at 4° C. for 30 min. After washing three times with PBS containing 1% BSA (PBS/BSA), cells were stained with Alexa647 Goat Anti-Human IgG Fc (Jackson ImmunoResearch, 109-605-098). After incubation, cells were washed three times with PBS/BSA and analyzed on iQue screener (Sartorius).

AMRA3-7D_UCHT1_scDb
DIVRSDIQMTQSPSSLSASVGDRVTITCRASQSVSSAVAWYQQKP
GKAPKLLIYSASSLYSGVPSRFSGSRSGTDFTLTISSLQPEDFAT
YYCQQISYVKKLITFGQGTKVEIKGGGGSEVQLQQSGPELVKPGA
SMKISCKASGYSFTGYTMNWVKQSHGKNLEWMGLINPYKGVSTYN
QKFKDKATLTVDKSSSTAYMELLSLTSEDSAVYYCARSGYYGDSD
WYFDVWGQGTTLTVSSGGGGSGGGGSGGGGSDIQMTQTTSSLSAS
LGDRVTISCRASQDIRNYLNWYQQKPDGTVKLLIYYTSRLHSGVP
SKFSGSGSGTDYSLTISNLEQEDIATYFCQQGNTLPWTFAGGTKL
EIKGGGGSEVQLVESGGGLVQPGGSLRLSCAASGFTFSDYSIHWV
RQAPGKGLEWVASISSSSGSTSYADSVKGRFTISADTSKNTAYLQ
MNSLRAEDTAVYYCARGGWYPAMDYWGQGTLVTVSSLEGGGGLND
IFEAQKIEWHESRHHHHHH
AMRA3-7D_CrossMab
>Chain A. QMY30735.1
EVQLLESGGGLVQPGGSLRLSCAASGFTFSTYAMNWVRQAPGKGL
EWVSRIRSKYNNYATYYADSVKGRFTISRDDSKNTLYLQMNSLRA
EDTAVYYCVRHGNFGNSYVSWFAYWGQGTLVTVSSASVAAPSVFI
FPPSDEQLKSGTASVVCLLNNFYPREAKVQWKVDNALQSGNSQES
VTEQDSKDSTYSLSSTLTLSKADYEKHKVYACEVTHQGLSSPVTK
SFNRGEC
>Chain B.
EVQLVESGGGLVQPGGSLRLSCAASGFTFSDYSIHWVRQAPGKGL
EWVASISSSSGSTSYADSVKGRFTISADTSKNTAYLQMNSLRAED
TAVYYCARGGWYPAMDYWGQGTLVTVSSASTKGPSVFPLAPSSKS
TSGGTAALGCLVEDYFPEPVTVSWNSGALTSGVHTFPAVLQSSGL
YSLSSVVTVPSSSLGTQTYICNVNHKPSNTKVDEKVEPKSCDGGG
GSGGGGSQAVVTQEPSLTVSPGGTVTLTCGSSTGAVTTSNYANWV
QEKPGQAFRGLIGGTNKRAPGTPARFSGSLLGGKAALTLSGAQPE
DEAEYYCALWYSNLWVFGGGTKLTVLSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPEAAGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALGAPIEKTISKAKGQPREPQVYTLPPCRDELT
KNQVSLWCLVKGFYPSDIAVEWESNGQPENNYKTTPPVLDSDGSF
FLYSKLTVDKSRWQQGNVFSCSVMHEALHNHYTQKSLSLSP
>Chain C.
EVQLVESGGGLVQPGGSLRLSCAASGFTFSDYSIHWVRQAPGKGL
EWVASISSSSGSTSYADSVKGRFTISADTSKNTAYLQMNSLRAED
TAVYYCARGGWYPAMDYWGQGTLVTVSSASTKGPSVFPLAPSSKS
TSGGTAALGCLVEDYFPEPVTVSWNSGALTSGVHTFPAVLQSSGL
YSLSSVVTVPSSSLGTQTYICNVNHKPSNTKVDEKVEPKSCDKTH
TCPPCPAPEAAGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALGAPIEKTISKAKGQPREPQVCTLPPSRDELT
KNQVSLSCAVKGFYPSDIAVEWESNGQPENNYKTTPPVLDSDGSF
FLVSKLTVDKSRWQQGNVFSCSVMHEALHNHYTQKSLSLSP
>Chain D.
DIQMTQSPSSLSASVGDRVTITCRASQSVSSAVAWYQQKPGKAPK
LLIYSASSLYSGVPSRFSGSRSGTDFTLTISSLQPEDFATYYCQQ
ISYVKKLITFGQGTKVEIKRTVAAPSVFIFPPSDRKLKSGTASVV
CLLNNFYPREAKVQWKVDNALQSGNSQESVTEQDSKDSTYSLSST
LTLSKADYEKHKVYACEVTHQGLSSPVTKSFNRGEC

FIG. 21 shows the cytotoxic effect of AMRA3-7D in the single-chain Diabody (scDb) format. (A) Raji cells were first pulsed with AMG-510 conjugated to a peptide corresponding to a fragment of KRAS(G12C) or a control peptide corresponding to KRAS (wild type). Pulsed cells were co-cultured with human T cells (Effector:Target=3:1) in the presence of scDb at the indicated concentrations. After incubation for 17 hours, dead cells were stained and detected by flow cytometry. (B) H2122 cells were first incubated with AMG-510 or DMSO. The cells were then co-cultured with human T cells (Effector:Target=10:1) in the presence of scDb at the indicated concentrations. After incubation, dead cells were measured by detecting a distinct intracellular protease activity released from membrane-compromised cells. Data shown here are from triplicate measurements. Error bars indicate the s.d. Where error bars are not visible, the errors are smaller than the symbols. Anti-HLA-A3 is a positive control.

FIG. 22 shows the CD3 binding properties and cytotoxic effects of AMRA3-7D in the CrossMab format. (A) Binding titration curve of AMRA3-7 CrossMab to Jurkat (CD3 positive) and Raji (CD3 negative) cells. The apparent K_{D} value is shown. Note that cells were not pulsed with any peptides. (B, C) Cytotoxic effects of AMRA3-7D CrossMab on Raji cells pulsed with an exogenous peptide-drug conjugate (B) and on H2122 cells treated with the drug (C). Methods are the same as in FIG. 22. Data shown here are from triplicate measurements. Error bars indicate the s.d. Where error bars are not visible, the errors are smaller than the symbols. Anti-HLA-A3 serves as a positive control.

### EXAMPLE 6

This example demonstrates deep mutational analysis of the AMR-A3-7D and OEA2-5 antibodies. Data from non-limiting embodiments is presented in FIGS. 23 and 24.

To identify mutations in CDRs of AMR-A3-7D and OEA2-5 that retain antigen binding, we performed deep mutational scanning on residues CDR-L3 and CDR-H3. In the yeast display format, each of the CDR-L3 and CDR-H3 residues were mutated to all genetically encoded amino acids using the NNK codon (N=A, T, G and C; K=G and T), one residue at a time. The resulting pool of mutants was combined, and the library was subjected to FACS using the relevant antigen, i.e., AMG510-KRAS(G12C) peptide in complex with HLA-A*03:01 for AMR-A3-7D and osimertinib-EGFR in complex with HLA-A*02:01. We used different antigen concentrations in order to adjust the stringency of library sorting. Vectors recovered from binding-capable and binding-incapable pools were analyzed by deep sequencing on an Illumina MiSeq instrument. Mutations found in different pools were deduced from the DNA sequencing analysis.

From this analysis, the disclosure provides the following permissible mutations at each CDR position as shown in the tables below. As references, the VL and VH sequences of the parent clones are shown, with the analyzed CDR residues in bold and italics. In embodiments, the disclosure includes each mutation alone, and all combination of mutations. Thus, as evident from the Tables, the disclosure included the described CDRs with 1, 2, 3, 4, 5, 6, 7, or 8 mutations as indicated in the Tables. The disclosure includes additional amino acid chances, such as in CDR1, CDR2, and in the framework sequences.

AMRA3-7D
V_{L}:
DIQMTQSPSS LSASVGDRVT ITCRASQSVS SAVAWYQQKP
GKAPKLLIYS ASSLYSGVPS RFSGSRSGTD FTLTISSLQP
EDFATYYCQQ ISYVKKLITF GQGTKVEIKR TV
V_{H}:
EVQLVESGGG LVQPGGSLRL SCAASGFTFS DYSIHWVRQA
PGKGLEWVAS ISSSSGSTSY ADSVKGRFTI SADTSKNTAY
LQMNSLRAED TAVYYCARGG WYPAMDYWGQ GTL VTVSS

TABLE A
Parental and consensus sequences d

| | Parental | | |
|---|---|---|---|
| Position | amino acid | | Consensus amino |
| (VL) | residue | Permissible mutation | acid residue |
| 91 | I | A, L, P, S, T, V | A, I, L, P, S, T, V |
| 92 | S | K, R, T | K, R, S, T |
| 93 | Y | F | F, Y |
| 94 | V | I, R, T | I, R, T, V |
| 95 | K | A, E, H, L, M, N, Q, | A, E, H, K, L, M, N, |
| | | R, S, T, Y | Q, R, S, T, Y |
| 96 | K | R | K, R |
| 97 | L | A, C, D, E, G, H, K, | A, C, D, E, G, H, K, |
| | | M, N, P, Q, R, S, T, | L, M, N, P, Q, R, S, |
| | | V, W | T, V, W |
| 98 | I | L, V | I, L, V |

TABLE B
Parental and consensus sequences

| | Parental | | |
|---|---|---|---|
| Position | amino acid | | Consensus amino |
| (VH) | residue | Permissible mutation | acid residue |
| 99 | G | | G |
| 100 | G | A, C, E, H, K, L, M, | A, C, E, G, H, K, L, M, |
| | | N, P, Q, R, S, T, W, Y | N, P, Q, R, S, T, W, Y |
| 101 | W | G, P, R, T | G, P, R, T, W |
| 102 | Y | A, D, E, F, G, H, I, K, | A, D, E, F, G, H, I, K, |
| | | M, N, Q, R, S, T, W | M, N, Q, R, S, T, W, Y |
| 103 | P | A, V | A, P, V |
| 104 | A | C, G, K, L, M, Q, R, | A, C, G, K, L, M, Q, R, |
| | | S, T, Y | S, T, Y |

### Deep Mutational Scanning of OEA2-5

OEA2-5
V_{L}:
DIQMTQSPSS LSASVGDRVT ITCRASQSVS SAVAWYQQKP
GKAPKLLIYS ASSLYSGVPS RFSGSRSGTD FTLTISSLQP
EDFATYYCQQ YSYWPITFGQ GTK VEIKRTV
V_{H}:
EVQLVESGGG LVQPGGSLRL SCAASGFTIS SSYIHWVRQA
PGKGLEWVAY ISPSYGSTSY ADSVKGRFTI SADTSKNTAY
LQMNSLRAED TAVYYCAREY VTMALDYWGQ GTLVTVSS

TABLE C
Parental and consensus sequences

| | Parental | | |
|---|---|---|---|
| Position | amino acid | | Consensus amino |
| (VL) | residue | Permissible mutation | acid residue |
| 91 | Y | | Y |
| 92 | S | A | A, S |
| 93 | Y | A, C, D, E, F, G, H, | A, C, D, E, F, G, H, |
| | | I, K, L, M, N, Q, R, | I, K, L, M, N, Q, R, |
| | | S, T, V, W | S, T, V, W, Y |
| 94 | W | | W |
| 95 | P | | P |
| 96 | I | E, P | E, I, P |

TABLE D
Parental and consensus sequences

| | Parental | | |
|---|---|---|---|
| Position | amino acid | | Consensus amino |
| (VH) | residue | Permissible mutation | acid residue |
| 99 | E | D | D, E |
| 100 | Y | E, F, L, Q, S, W | E, F, L, Q, S, W, Y |
| 101 | V | T, I | I, T, V |
| 102 | T | A, E, N, S | A, E, N, S, T |
| 103 | M | F, Y | F, M, Y |
| 104 | A | C, G, S, T | A, C, G, S, T |
| 105 | L | A, C, E, F, H, I, K, | A, C, E, F, H, I, K, L, |
| | | M, N, P, Q, S, T, V | M, N, P, Q, S, T, V |

FIG. 23 describes deep mutational scanning analysis of the CDR-L3 and H3 residues of AMR-A3-7D. (a) Representative flow cytometry profiles of yeast cells displaying AMRA3-7D or its deep mutational scanning library populations. Binding to 5 nM HLA-A*03:01 presenting AMG-510 conjugated to the Cys residue in the peptides, VVGACGVGK, was measured. The profile of the parental antibody, AMRA3-7D, is shown on the left, and those for sorted subsets of the deep mutational scanning library are shown on the right. The library was sorted with 1, 3, and 10 nM target, referred to as conditions 1, 2 and 3, respectively, and the nonbinder pool was sorted with 50 nM target. (b) The prevalence of mutations in the sorted subsets of the deep mutational scanning library is presented in a heat map format. The number of deep sequencing reads were normalized to the total reads for each sorted pool and multiplied by 1000. Crosses indicate the wild-type amino acid.

FIG. 24 describes results from deep mutational scanning analysis of CDR-L3 and H3 residues of OEA2-5. (a) Representative flow cytometry profiles of yeast cells displaying OEA2-5 in single-chain Fv format and its deep mutational scanning library populations. Binding to 1.5 nM streptavidin tetramer saturated with biotinylated HLA-A*02:01 presenting osimertinib conjugated to the Cys residue in the peptides, QLMPFGCLL, was measured. The profile of the parental antibody, OEA2-5, is shown on the left, and those for sorted subsets of the deep mutational scanning library are shown on the right. The library was sorted with 12.5, 2.5, and 0.5 nM target, referred to as conditions 1, 2 and 3, respectively, and the nonbinder pool was sorted with 12.5 nM target. (b) The prevalence of mutations in the sorted subsets of the deep mutational scanning library is presented in a heat map format. The number of deep sequencing reads were normalized to the total reads for each sorted pool and multiplied by 1000. Crosses indicate the wild-type amino acid.

### EXAMPLE 7

This example discloses novel binding partner sequences that recognize drug-peptide conjugates in the context of human MHC (major histocompatibility complex) molecules (also referred to as human leukocyte antigens (HLAs) in human), as further described above. These novel binding partners exhibit improved properties relative to the binding partners described in the above examples, in that they bind to the drug-peptide/HLA complexes with greater affinity. In the case of KRAS(G12C) antibodies, they bind to two different peptides derived from KRAS(G12C) conjugated to a drug (AMG-510, a.k.a. sotorasib) presented on two different HLAs, which together substantially broaden the range of targets that each antibody can recognize.

This Example also describes a set of T-cell engager constructs that effectively kill otherwise sotorasib-resistant cancer cells in a drug treatment- and T cell-dependent manner. These results validate the described HapImmune™ concept and the KRAS(G12C) approach.

This example also describes preparation and use of binding partner sequences that comprise combinations of permissible mutations, which mutations are individually described in herein, including but not necessarily limited to Tables E and F as shown in the Figures. The disclosure includes all combinations of the described mutations. The binding partners may be an intact antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BiTE), bispecific killer cell engager (BiKE), CrossMab, a camelid antibody, a tri-specific binding partner, or chimeric antigen receptor (CAR).

Methods. NCI-H2122 cells (from ATCC) expressing luciferase were cultured in RPMI supplemented with 10% fetal bovine serum (FBS), penicillin/streptomycin and 10 μg/mL Blasticidin. For drug-treated cells, H2122 cells were cultured with 1 mM AMG-510 for more than a week. For the cytotoxicity assay, 1×10⁴ cells/well were seeded in a 96-well flat bottom plate and incubated at 37° C. for 24 hours. After incubation, the cells were co-cultured with human T cells (E:T=10:1) and scDbs in the presence of 1 mM AMG-510 or DMSO, 2 μg/mL human beta-2 microglobulin and 10 ng/mL IL-7 and IL-15 for 24 hours at 37° C. The supernatant of each well was transferred into a 96-well plate, then Coelenterazine was added to the wells. Luminescence was measured using Synergy Neo2 hybrid multi-mode reader (BioTek).

Sequences for exemplary binding partners are provided below:

The following are heavy chain variable region and light chain variable region sequences of exemplary antibodies targeting AMG510-KRAS^{G12C} peptide conjugates presented on HLA-A*03:01 and HLA-A*11:01.

• AMRA3-7D (reference binding partner)
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQISYVKKLITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGGWYPAMDYWGQGTLV
TVSS
• RA_D01
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRRTITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGGWIAAMDYWGQGTLVTVS
S
• RA_D02
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQPSYVRRKITFGQGTKVEIKRTV
V_{H}:
EVQLVESGGGLVQPGGSLRLSCAASGFTESDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D03
V_{L}:
DIQMTQSPSSLSASVGDRVTITCRASQSVSSAVAWYQQKPGKAPKLLIYSASSLYSG
VPSRESGSRSGTDFTLTISSLQPEDFATYYCQQASYVARKITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGGWIAAMDYWGQGTLVTVS
S
• RA_D04
V_{L}:
DIQMTQSPSSLSASVGDRVTITCRASQSVSSAVAWYQQKPGKAPKLLIYSASSLYSG
VPSRESGSRSGTDFTLTISSLQPEDFATYYCQQVSYVARRITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D06
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVKRLITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGHWIAAMDYWGQGTLVTVS
S
• RA_D07
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRRLITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D08
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRRVITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGGWIAAMDYWGQGTLVTVS
S
• RA_D09
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRRTITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGSWIAAMDYWGQGTLVTVS
S
• RA_D11
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRKTITFGQGTKVEIKRTV
V_{H}:
EVQLVESGGGLVQPGGSLRLSCAASGFTESDYSIHWVRQAPGKGLEWVASISSSSGS
TSYADSVKGRFTISADTSKNTAYLQMNSLRAEDTAVYYCARGGWIAAMDYWGQGTLVTVS
S
• RA_D12
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVAKTITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D13
V_{L}:
DIQMTQSPSSLSASVGDRVTITCRAGQSVSSAVAWYQQKPGKAPKLLIYSASSLYSG
VPSRFSGSRSGTDFTLTISSLQPEDFATYYCQQSSYVRRKITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGKWIPAMDYWGQGTLVTVS
S
• RA_D14
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRKAITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGGWIAAMDYWGQGTLVTVS
S
• RA_D16
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRRAITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D18
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQSSYVKRTITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D19
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQPSYVRKTITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D20
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVKKEITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D21
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQSSYVHKLITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S
• RA_D23
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVRREITFGQGTKVEIKRTV
V_{H}:
EVQLVESGGGLVQPGGSLRLSCAASGFTESDYSIHWVRQAPGKGLEWVASISSSSGS
TSYADSVKGRFTISADTSKNTAYLQMNSLRAEDTAVYYCARGHWIAAMDYWGQGTLVTVS
S
• RA_D24
V_{L}:
DIQMTQSPSSLSASVGDRVTITCRASQSVSSAVAWYQQKPGKAPKLLIYSASSLYSG
VPSRFSGSRSGTDFTLTISSLQPEDFATYYCQQASYVHRLITFGQGTKVEIKRTV
V_{H}:
EVQLVESGGGLVQPGGSLRLSCAASGFTFSDYSIHWVRQAPGKGLEWVASISSSSGS
TSYADSVKGRFTISADTSKNTAYLQMNSLRAEDTAVYYCARGRWIPAMDYWGQGTLVTVS
S

### Exemplary Single-Chain Diabody Clones Targeting AMG510-KRAS(G12C) Peptide Conjugates Presented on HLA-A*03:01 and HLA-A*11:01

The underlined sequences immediately below represent AviTag and HisTag, respectively, and may be excluded from the binding partners of this disclosure.

• RA_D01_UCHT1_scDb
DIVRSDIQMTQSPSSLSASVGDRVTITCRASQSVSSAVAWYQQKPGKAPKLLIYSAS
SLYSGVPSRESGSRSGTDFTLTISSLQPEDFATYYCQQASYVRRTITFGQGTKVEIK
GGGGSEVQLQQSGPELVKPGASMKISCKASGYSFTGYTMNWVKQSHGKNLEWMGLIN
PYKGVSTYNQKFKDKATLTVDKSSSTAYMELLSLTSEDSAVYYCARSGYYGDSDWYF
DVWGQGTTLTVSSGGGGSGGGGSGGGGSDIQMTQTTSSLSASLGDRVTISCRASQDI
RNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTESD
YSIHWVRQAPGKGLEWVASISSSSGSTSYADSVKGRFTISADTSKNTAYLQMNSLRA
EDTAVYYCARGGWIAAMDYWGQGTLVTVSSLEGGGGLNDIFEAQKIEWHESRHHHHHH
• RA_D08_UCHT1_scDb
DIVRSDIQMTQSPSSLSASVGDRVTITCRASQSVSSAVAWYQQKPGKAPKLLIYSAS
SLYSGVPSRFSGSRSGTDFTLTISSLQPEDFATYYCQQASYVRRVITFGQGTKVEIK
GGGGSEVQLQQSGPELVKPGASMKISCKASGYSFTGYTMNWVKQSHGKNLEWMGLIN
PYKGVSTYNQKFKDKATLTVDKSSSTAYMELLSLTSEDSAVYYCARSGYYGDSDWYF
DVWGQGTTLTVSSGGGGSGGGGSGGGGSDIQMTQTTSSLSASLGDRVTISCRASQDI
RNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTFSD
YSIHWVRQAPGKGLEWVASISSSSGSTSYADSVKGRFTISADTSKNTAYLQMNSLRA
EDTAVYYCARGGWIAAMDYWGQGTLVTVSSLEGGGGLNDIFEAQKIEWHESRHHHHHH
• RA_D11_UCHT1_scDb
DIVRSDIQMTQSPSSLSASVGDRVTITCRASQSVSSAVAWYQQKPGKAPKLLIYSAS
SLYSGVPSRFSGSRSGTDFTLTISSLQPEDFATYYCQQASYVRKTITFGQGTKVEIK
GGGGSEVQLQQSGPELVKPGASMKISCKASGYSFTGYTMNWVKQSHGKNLEWMGLIN
PYKGVSTYNQKFKDKATLTVDKSSSTAYMELLSLTSEDSAVYYCARSGYYGDSDWYF
DVWGQGTTLTVSSGGGGSGGGGSGGGGSDIQMTQTTSSLSASLGDRVTISCRASQDI
RNYLNWYQQKPDGTVKLLIYYTSRLHSGVPSKESGSGSGTDYSLTISNLEQEDIATY
FCQQGNTLPWTFAGGTKLEIKGGGGSEVQLVESGGGLVQPGGSLRLSCAASGFTFSD
YSIHWVRQAPGKGLEWVASISSSSGSTSYADSVKGRFTISADTSKNTAYLQMNSLRA
EDTAVYYCARGGWIAAMDYWGQGTLVTVSSLEGGGGLNDIFEAQKIEWHESRHHHHHH

This disclosure includes the sequences of the scDbs RA_D01_UCHT1_scDb, RA_D08_UCHT1_scDb, and RA_D11_UCHT1_scDb without the underlined AviTag and HisTag sequences. Non-underlined amino acids between underlined sequences represent linkers. The underlined sequences may be excluded from the sequences of this disclosure here and elsewhere unless indicated otherwise and where underlined sequences are presented but are not CDR sequences. The same applies to non-underlined sequences that intervene such underlined non-CDR sequences.

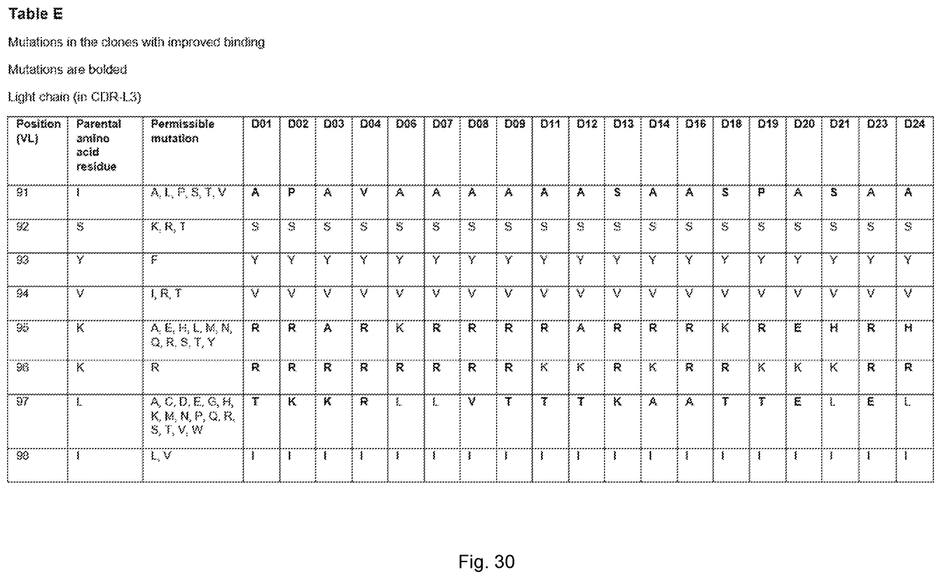

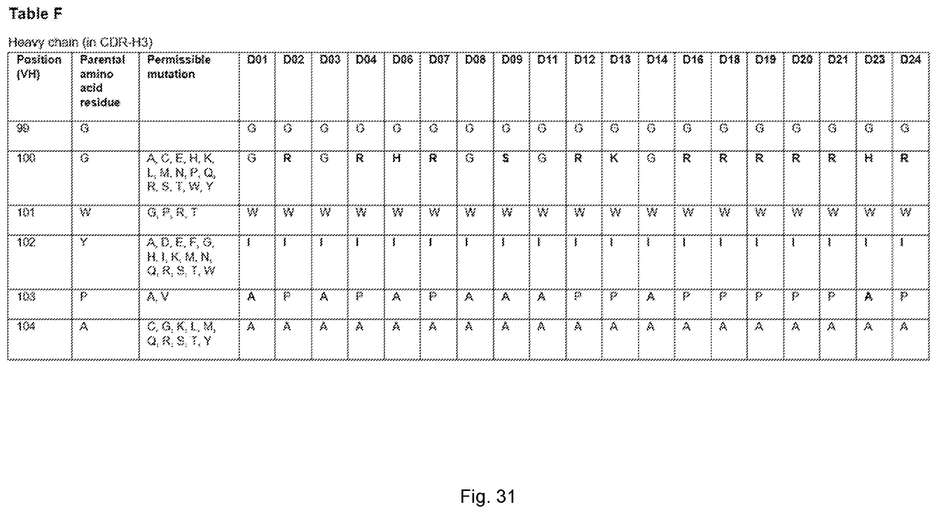

The combinations of permissible mutations using sequence of AMRA3-7D as a reference, are shown in Tables E and F, which are presented as FIGS. 30 and 31 and the tables in those figures, respectively.

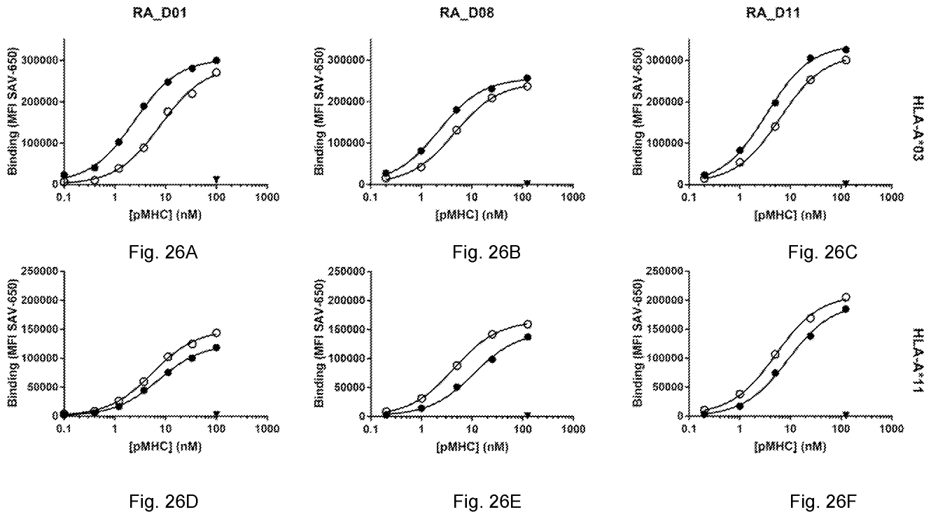

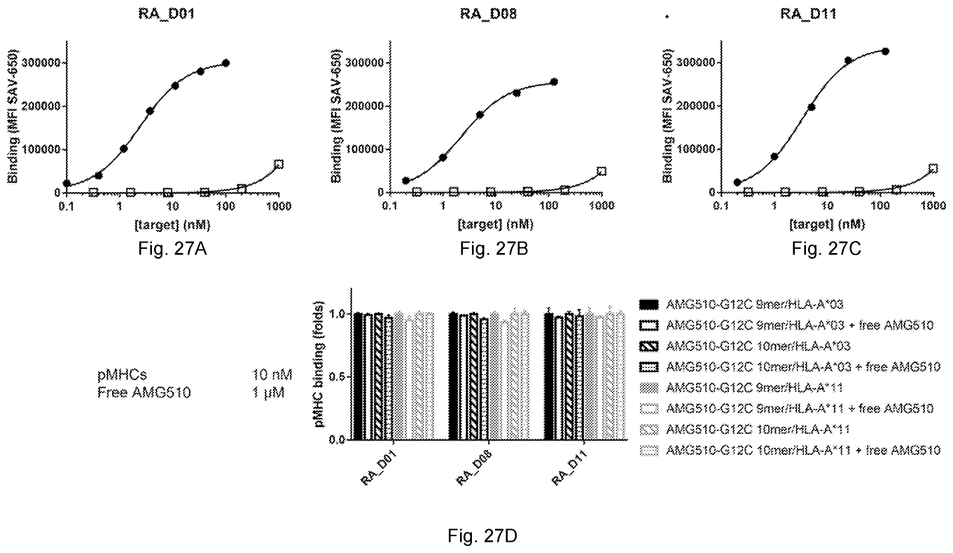

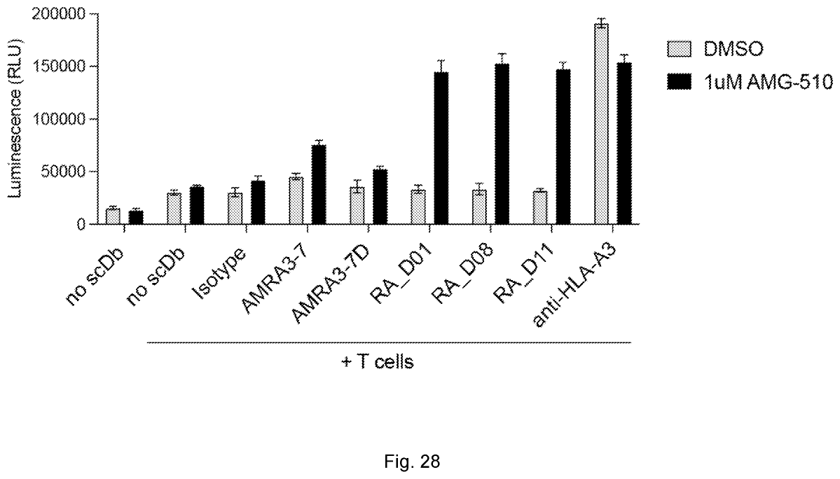

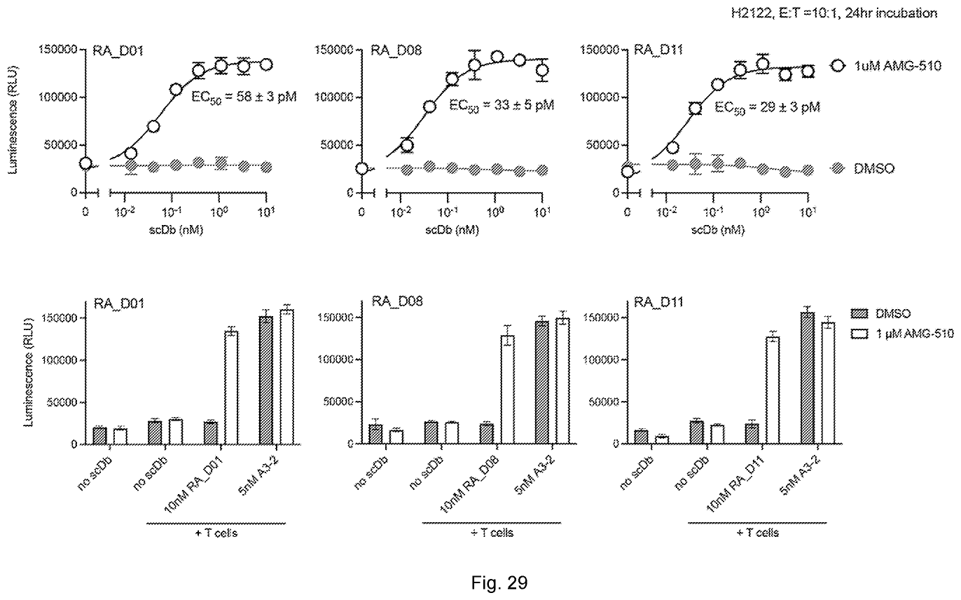

Results of binding for the various antibody clones are shown in FIGS. 25-29. FIG. 25 shows that the binding of all the antibody clones (measured as median fluorescence intensity) was higher than the reference AMRA3-7D. FIG. 26 shows specific binding of the antibodies for conjugates of AMG-510 with two different peptides of KRAS G12C in the context of two different HLAs (HLA-A*03 and HLA-A*11). FIG. 27 panels A-C show that these antibodies show marginal binding to the peptide-AMG conjugate in the absence of HLA presentation. FIG. 27 panel D shows that these antibodies are not inhibited by the presence of excess concentrations of free AMG510. FIG. 28 shows that the affinity-matured antibodies showed potent cytotoxic effect in a manner dependent on AMG-510 treatment. FIG. 29 shows the cytotoxic effect of some of the scDb antibodies compared to control scDb on AMG510-treated lung cancer cells. The positive control is an scDb that contains a scFv sequence that binds HLA-A*03 regardless of the presented peptide.

The present antibodies can be used to deplete cells that have the target (KRAS G12C) upon the treatment with a covalent inhibitor (e.g., AMG510). The HapImmune™ approach achieves exquisite specificity by create an AND logic from (i) drug treatment, (ii) MHC presentation, and (iii) antibody treatment. It can also result in reduction of the drug dose and potentially temporal control of immune-associated adverse effects by withholding a drug.

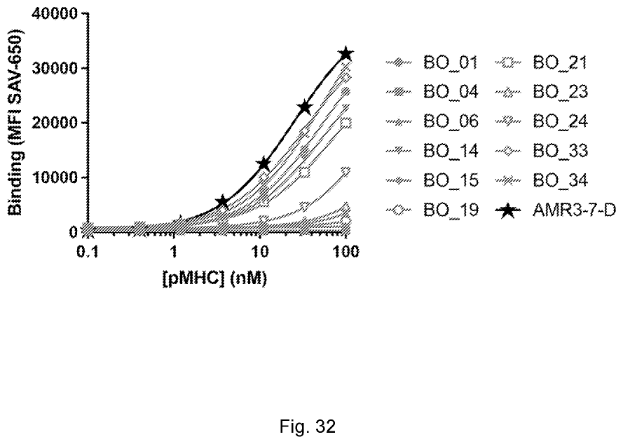

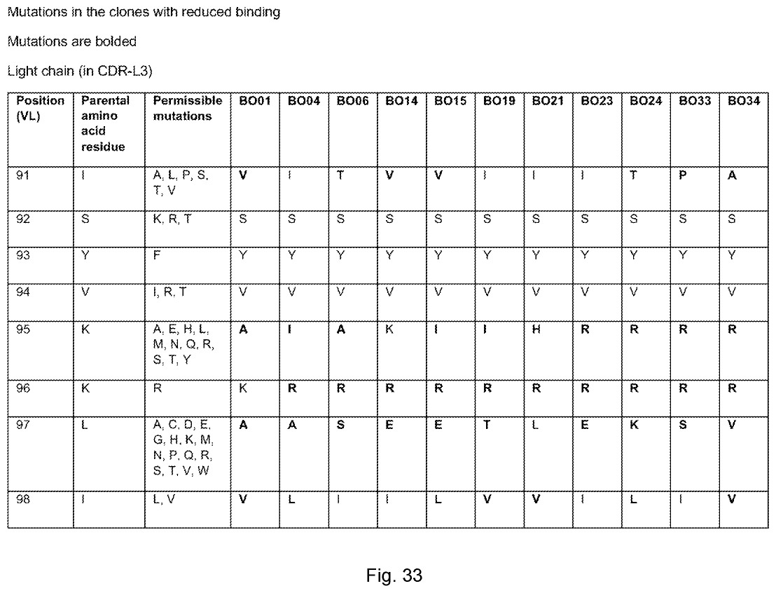

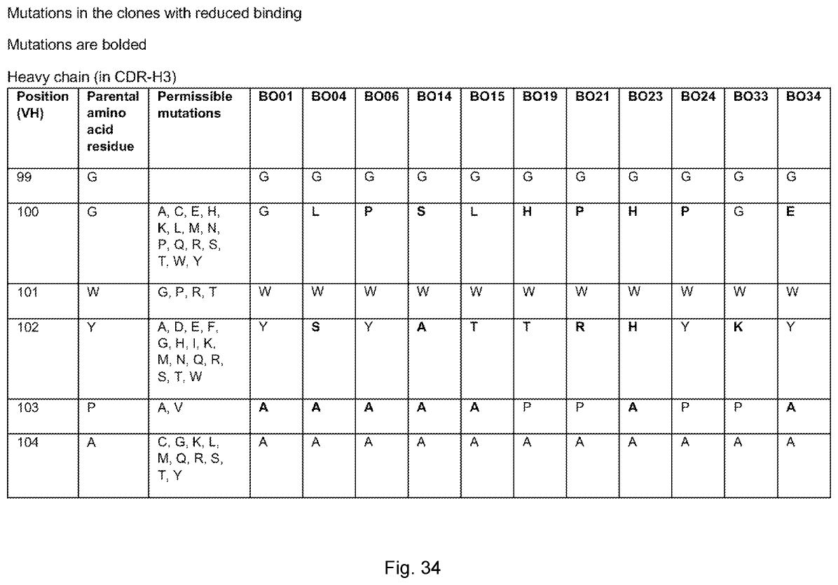

Additionally, we identified antibodies having sequences consistent with the consensus sequences described in Tables A and B that exhibit reduced affinity to AMG510-KRAS(G12C) peptide conjugates presented on HLA-A*03:01. FIG. 32 shows binding titrations demonstrating the reduced affinity of these antibodies relative to the parental clone, AMRA3-7D. FIGS. 33 and 34 show the amino acid sequences of the relevant CDRs of these clones, confirming that these sequences fall within the consensus sequences. Comparison of the sequences of the reduced affinity clones with the sequences of other antibodies described herein provides information regarding permissible changes that maintain affinity for the described peptide conjugates and remain part of the consensus sequences. The sequences of the reduced affinity clones shown in FIG. 32 are:

• BO01
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQVSYVAKAVTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGEWYAAMDYWGQGTLVTVSS
• BO04
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQISYVIRALTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGLWSAAMDYWGQGTLVTVSS
• BO06
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQTSYVARSITFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGPWYAAMDYWGQGTLVTVSS
• BO14
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQVSYVKREITFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGSWAAAMDYWGQGTLVTVSS
• BO15
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQVSYVIRELTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGLWTAAMDYWGQGTLVTVSS
• BO19
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQISYVIRTVTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGHWTPAMDYWGQGTLVTVSS
• BO21
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQISYVHRLVTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGPWRPAMDYWGQGTLVTVSS
• BO23
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQISYVRREITFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGHWHAAMDYWGQGTLVTVSS
• BO24
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQTSYVRRKLTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGPWYPAMDYWGQGTLVTVSS
• BO33
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQPSYVRRSITFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWKPAMDYWGQGTLVTVSS
• BO34
V_{L}:
DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRVVTFGQGTKVEIKRTV
V_{H}:
EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGEWYAAMDYWGQGTLVTVSS

We further show that one of the improved antibody clones, RA_D11, binds to AMG510-KRAS(G12C) peptide conjugates presented on any of three different HLAs. RA_D11 was produced as purified Fab, and its binding was examined using biolayer interferometry (BLI) on an Octet instrument (Sartorius). Consistent with its characterization in the yeast display format (FIG. 26), the RA_D11 Fab binds to AMG510-KRAS(G12C) peptide conjugates presented on HLA-A*03 and HLA-A*11 with high affinity, with K_{D} values in the sub nM and single nM ranges.

Importantly, RA_D11 Fab also binds to AMG510-KRAS(G12C) peptide conjugates presented on HLA-A*02 with only slightly reduced affinity. HLA-A*02 and HLA-A*03/11 have distinct preferences for peptide presentation. Consequently, the peptide used for HLA-A*02, KLVVVGAC*GV, is distinct from those for HLA-A*03/11, VVVGAC*GVGK. These results indicate that an antibody can be developed that binds to one covalent drug that is conjugated to different peptides and presented on correspondingly different HLA molecules, while maintaining specificity to the peptide conjugation on HLA over the free drug.

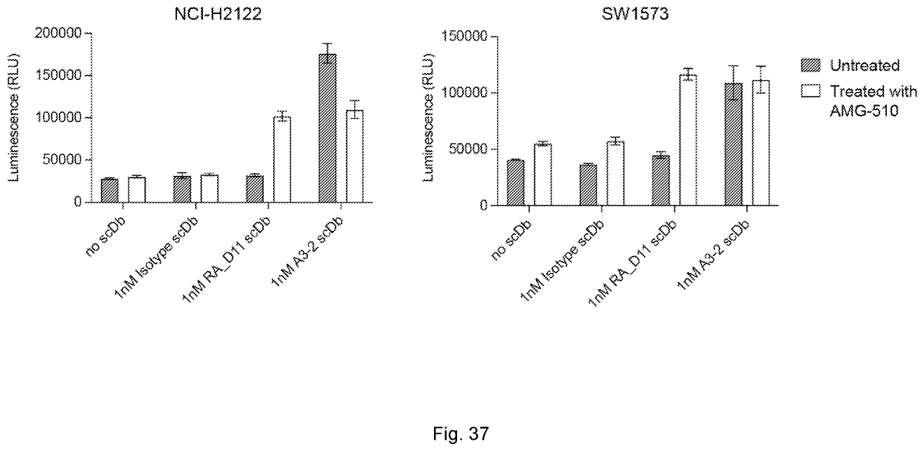

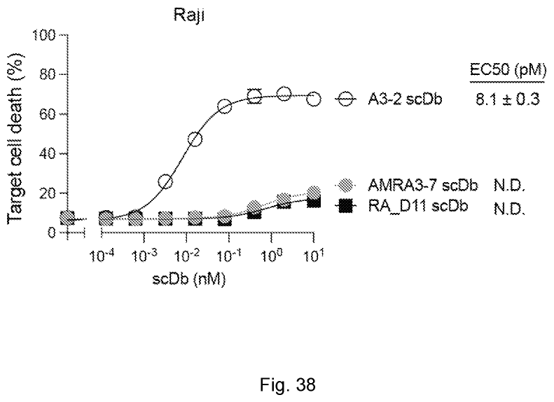

The ability of RA_D11 to bind to AMG510-KRAS(G12C) peptide conjugates presented on HLA-A*02 suggests that a RA_D11 scDb antibody might have cytotoxic activity on cells that express KRAS(G12C) and HLA-A*02 and are treated with AMG510. FIG. 37 shows that the RA_D11 scDb antibody indeed shows selective cytotoxic effect on the KRAS(G12C)-positive lung cancer cell line, SW1573, which expresses HLA-A*02 and HLA-A*24 (but not HLA-A*03 or HLA-A*11) in a manner dependent on AMG-510 pretreatment. FIG. 38 shows that, by contrast, the RA_D11 scDb antibody did not show cytotoxicity on Raji cells, which express HLA-A*03 but not KRAS(G12C), supporting the conclusion that the cytotoxic activity of the RA_D11 scDb antibody is not due to nonspecific recognition of AMG-510 (or another off-target molecule) presented in a manner independent of KRAS(G12C).

### EXAMPLE 8

This Example provides a description of additional binding partners that specifically bind to a conjugate formed by the covalent reaction of osimertinib-with an EGFR peptide presented on HLA-A*02.

The binding partners in this Example relate in part to affinity maturation of OEA2-5. The sequence of OAE2-5 is reproduced here for reference.

• OEA2-5
V_{L}:
DIQMTQSPSSLSASVGDRVTITCRASQSVSSAVAWYQQKPGKAPKLLIYS
ASSLYSGVPSRFSGSRSGTDFTLTISSLQPEDFATYYCQQYSYWPITFGQ
GTKVEIKRTV
V_{H}:
EVQLVESGGGLVQPGGSLRLSCAASGFTISSSYIHWVRQAPGKGLEWVAY
ISPSYGSTSYADSVKGRFTISADTSKNTAYLQMNSLRAEDTAVYYCAREY
VTMALDYWGQGTLVTVSS

Based on deep mutational scanning study of OEA2-5 (Table C and FIG. 24), we constructed a combinatorial library from which the following sequences with improved binding to osimertinib-EGFR presented on HLA-A*02.

### Affinity Matured Clones:

EO_Q01
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELVTMTADYWGQGTLVTVSS
EO_Q02
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSAWPETFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYTTMSIDYWGQGTLVTVSS
EO_Q03
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSAWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARESVTMSADYWGQGTLVTVSS
EO_Q04
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSDWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARESVTMTKDYWGQGTLVTVSS
EO_Q05
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYAEWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREEVTMSIDYWGQGTLVTVSS
EO_Q06
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARESITMTKDYWGQGTLVTVSS
EO_Q07
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYAKWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELTTMSIDYWGQGTLVTVSS
EO_Q08
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSSWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELTEMTTDYWGQGTLVTVSS
EO_Q09
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREEVTMTADYWGQGTLVTVSS
EO_Q10
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSGWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELIEMTPDYWGQGTLVTVSS
EO_Q11
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSGWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELVTMTIDYWGQGTLVTVSS
EO_Q12
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYASWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELTTMSIDYWGQGTLVTVSS
EO_Q13
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELTTMTADYWGQGTLVTVSS
EO_Q14
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSSWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARESITMSPDYWGQGTLVTVSS
EO_Q15
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELIEMTTDYWGQGTLVTVSS
EO_Q16
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSDWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYVTMSIDYWGQGTLVTVSS
EO_Q17
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSSWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCARELVEMTPDYWGQGTLVTVSS
EO_Q18
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSAWPETFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYVTMALDYWGQGTLVTVSS
EO_Q20
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSDWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREEVTMTPDYWGQGTLVTVSS
EO_Q22
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSHWPITFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYVTMALDYWGQGTLVTVSS
EO_Q23
V_{L}:
DIQMTQSPSSLSASVGDRVTITCRASQSVSSAVAWYQQKPGKAPKLLIYSASSLYSGVPSRESGSR
SGTDFTLTISSLQPEDFATYYCQQYSHWPETFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYVTMALDYWGQGTLVTVSS
EO_Q24
V_{L}:
DIQMTQSPSSLSASVGDRVTITCRASQSVSSAVAWYQQKPGKAPKLLIYSASSLYSGVPSRFSGSR
SGTDFTLTISSLQPEDFATYYCQQYSEWPETFGQGTKVEIKRTV
V_{H}:
EVQLVESGGGLVQPGGSLRLSCAASGFTISSSYIHWVRQAPGKGLEWVAYISPSYGSTSYADSVKG
RFTISADTSKNTAYLQMNSLRAEDTAVYYCAREYVTMALDYWGQGTLVTVSS

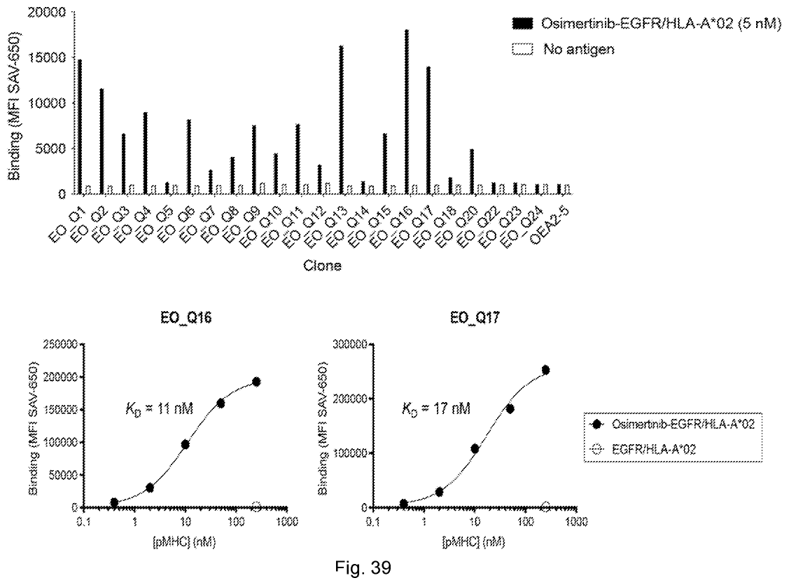

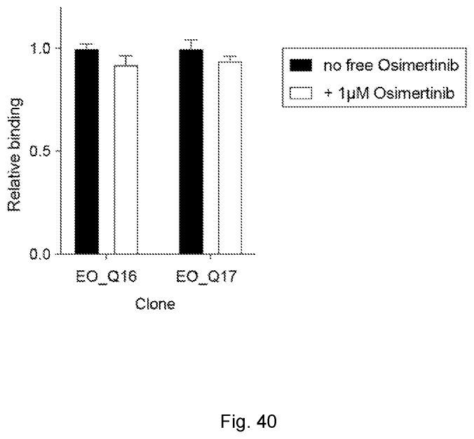

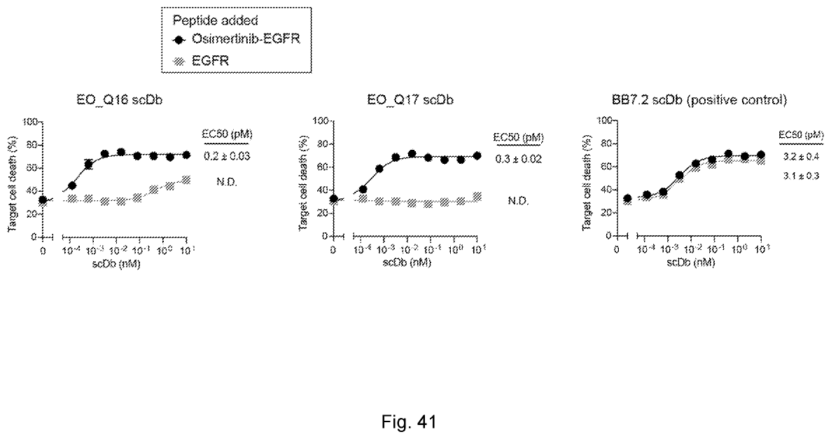

The binding partners described above in this Example show higher binding signals to osimertinib-EGFR/HLA-A*02, as tested in the yeast display format and as shown in FIGS. 39 and 40. In particular, FIG. 39 demonstrates results from binding titration of EO_Q16 and EO_Q17 to osimertinib-EGFR/HLA-A*02 (filled circles) and to EGFR/HLA-A*02 (no drug-conjugation; open circles), showing that these antibodies have high affinity to their intended antigen but not to the equivalent antigen without drug conjugation. FIG. 40 demonstrates results from binding of EO_Q16 and EO_Q17 to osimertinib-EGFR/HLA-A*02 (10 nM) in the absence and presence of free osimertinib (1 μM), showing that antigen binding of these antibodies is not inhibited by the presence of free drug.

We constructed the following representative single-chain Diabodies (scDbs) using these described in this Example as building blocks.
scDb sequences are as shown below. The underlined sequences represent AviTag and HisTag, respectively and can be excluded from the described sequences.

OEA2-5_UCHT1_scDb
DIVRSDIQMTQSPSSLSASVGDRVTITCRASQSVSSAVAWYQQKPGKAPKLLIYSASSLYS
GVPSRFSGSRSGTDFTLTISSLQPEDFATYYCQQYSYWPITFGQGTKVEIKGGGGSEVQLQ
QSGPELVKPGASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVWGQGTTLTVSS
GGGGSGGGGSGGGGSDIQMTQTTSSLSASLGDRVTISCRASQDIRNYLNWYQQKPDGTV
KLLIYYTSRLHSGVPSKFSGSGSGTDYSLTISNLEQEDIATYFCQQGNTLPWTFAGGTKLEI
KGGGGSEVQLVESGGGLVQPGGSLRLSCAASGFTISSSYIHWVRQAPGKGLEWVAYISPS
YGSTSYADSVKGRFTISADTSKNTAYLQMNSLRAEDTAVYYCAREYVTMALDYWGQGT
LVTVSSLEGGGGLNDIFEAQKIEWHESRHHHHHH
EO_Q16_UCHT1_scDb
DIVRSDIQMTQSPSSLSASVGDRVTITCRASQSVSSAVAWYQQKPGKAPKLLIYSASSLYS
GVPSRFSGSRSGTDFTLTISSLQPEDFATYYCQQYSDWPITFGQGTKVEIKGGGGSEVQLQ
QSGPELVKPGASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVWGQGTTLTVSS
GGGGSGGGGSGGGGSDIQMTQTTSSLSASLGDRVTISCRASQDIRNYLNWYQQKPDGTV
KLLIYYTSRLHSGVPSKFSGSGSGTDYSLTISNLEQEDIATYFCQQGNTLPWTFAGGTKLEI
KGGGGSEVQLVESGGGLVQPGGSLRLSCAASGFTISSSYIHWVRQAPGKGLEWVAYISPS
YGSTSYADSVKGRFTISADTSKNTAYLQMNSLRAEDTAVYYCAREYVTMSIDYWGQGTL
VTVSSLEGGGGLNDIFEAQKIEWHESRHHHHHH
EO_Q17_UCHT1_scDb
DIVRSDIQMTQSPSSLSASVGDRVTITCRASQSVSSAVAWYQQKPGKAPKLLIYSASSLYS
GVPSRFSGSRSGTDFTLTISSLQPEDFATYYCQQYSSWPITFGQGTKVEIKGGGGSEVQLQ
QSGPELVKPGASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVWGQGTTLTVSS
GGGGSGGGGSGGGGSDIQMTQTTSSLSASLGDRVTISCRASQDIRNYLNWYQQKPDGTV

KLLIYYTSRLHSGVPSKFSGSGSGTDYSLTISNLEQEDIATYFCQQGNTL
PWTFAGGTKLEIKGGGGSEVQLVESGGGLVQPGGSLRLSCAASGFTISSS
YIHWVRQAPGKGLEWVAYISPSYGSTSYADSVKGRFTISADTSKNTAYLQ
MNSLRAEDTAVYYCARELVEMTPDYWGQGTLVTVSSLEGGGGLNDIFEAQ
KIEWHESRHHHHHH

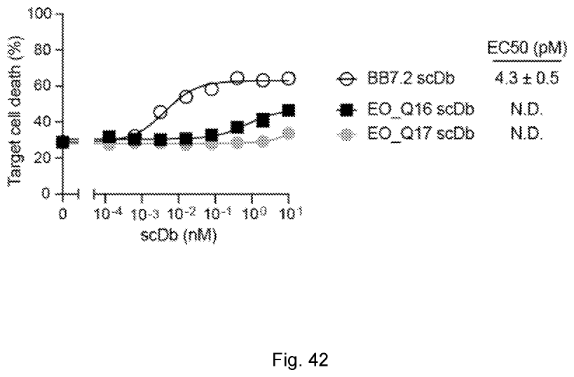

FIGS. 41 and 42 illustrate data obtained from testing the scDB antibodies. In particular, FIG. 41 shows cytotoxic effect of scDb antibodies on the cells spiked with the drug-peptide conjugates. EO_Q16 and EO_Q17 scDbs showed potent cytotoxic effects on the cells spiked with the osimertinib-EGFRb peptide conjugate but not on the cells treated with the same peptide without drug conjugation. BB7.2 scDb is an anti-pan HLA-A2 clone, which was used as a positive control. Data shown are from triplicate measurements.

FIG. 42 shows cytotoxic effects of scDb antibodies on OCI-AML3 cells treated with osimertinib. EO_Q16 and EO_Q17 scDb antibodies did not show cytotoxic effects, as expected because OCI-AML3 cells do not possess the target of osimertinib, an activated EGFR mutant. BB7.2 scDb is an anti-pan HLA-A2 clone, which was used as a positive control. Data shown are from triplicate measurements.

### Methods for FIGS. 41 and 42

OCI-AML3 cells were culture in IMDM supplemented with 20% fetal bovine serum (FBS) and penicillin/streptomycin. OCI-AML3 cells were first stained with carboxyfluorescein succinimidyl ester (CSFE, ThermoFisher, 65-0850-84), then incubated with the final 1 μM EGFR-osimertinib peptide, or 1 μM EGFR peptide pretreated with beta-mercaptoethanol (EGFR-bME) (FIG. 41) or 1 mM osimertinib (FIG. 42) in the presence of 10 μg/mL human beta-2 microglobulin for 4 hr. The cells were harvested using centrifugation and washed in the media. The cells were then co-cultured with human T-cells (E:T=5:1) in the presence of scDb for 18-19 hr. After incubation, cells were harvested and washed with PBS, then stained with Fixable Viability Dye eFluor660 (ThermoFisher, 65-0864-14). After washing cells, the cells were analyzed on iQue screener (Satorius) and the viability of CSFE-positive cells was assessed.

The amino acid sequence of BB7.2 (Parham P & Brodsky FM (1981) Partial purification and some properties of BB7.2. A cytotoxic monoclonal antibody with specificity for HLA-A2 and a variant of HLA-A28. Hum Immunol 3:277-299) was obtained from US20200283529A1, from which the amino acid and nucleotide sequences encoding BB7.2 are incorporated herein by reference.

### EXAMPLE 9

### Sotorasib-Treated Tumor Cells can be Killed Selectively by HapImmune™ Antibodies

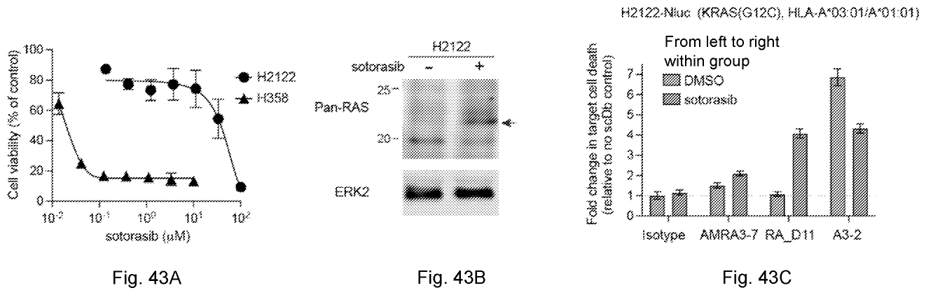

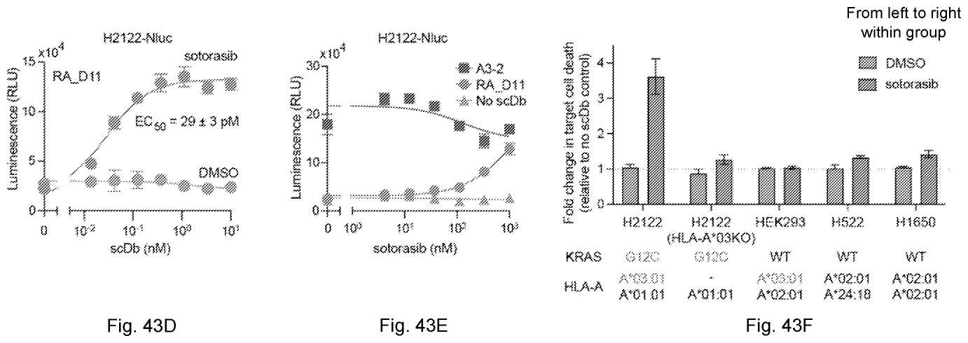

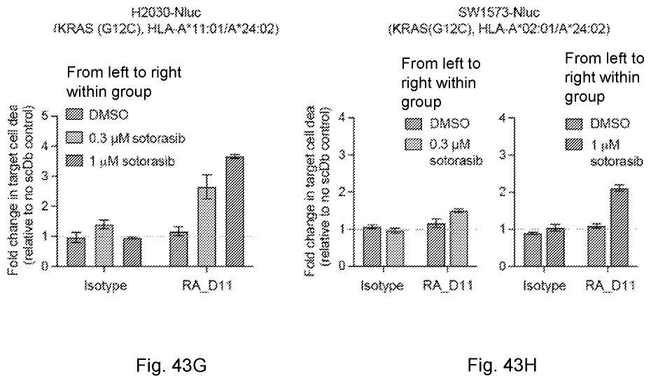

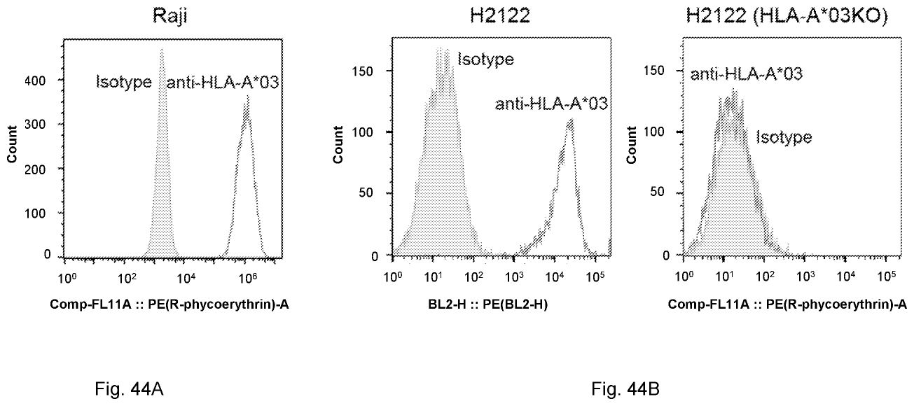

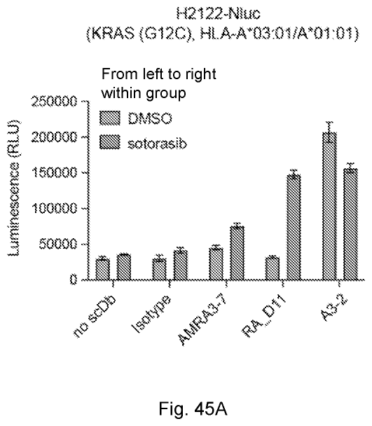

The ability of the RA_D11 scDb to target sotorasib-treated KRAS(G12C)-harboring tumor cells that are resistant to the inhibitor was analyzed. The NCI-H358 cell line commonly used for studies of sotorasib and other G12C inhibitors is highly sensitive to these agents and therefore is not suitable for evaluating the HapImmune approach. Instead, a sotorasib-insensitive cell line was identified, NCI-H2122 (hereafter H2122) that expresses KRAS(G12C) and HLA-A*03 (FIG. 44). H2122 is resistant to sotorasib at a concentration up to ˜10 μM (FIG. 43A), a therapeutically relevant concentration range, even though sotorasib a concentration as low as 0.1 μM fully engages KRAS(G12C) in these cells (FIG. 43B). By contrast, nearly all of the H358 cells were killed with 0.1 μM sotorasib (FIG. 43A). Therefore, the HapImmune™ scDb-induced killing was assessed in H2122 cells exposed to 0.1-1.0 M sotorasib. To specifically measure target cell death in the presence of T cells (some of which also die), a variant of H2122 cells, H2122-Nluc, with intracellular expression of NanoLuc was generated. Luciferase released into the media by dying cells can then be quantified, providing an accurate assessment of cancer cell death.

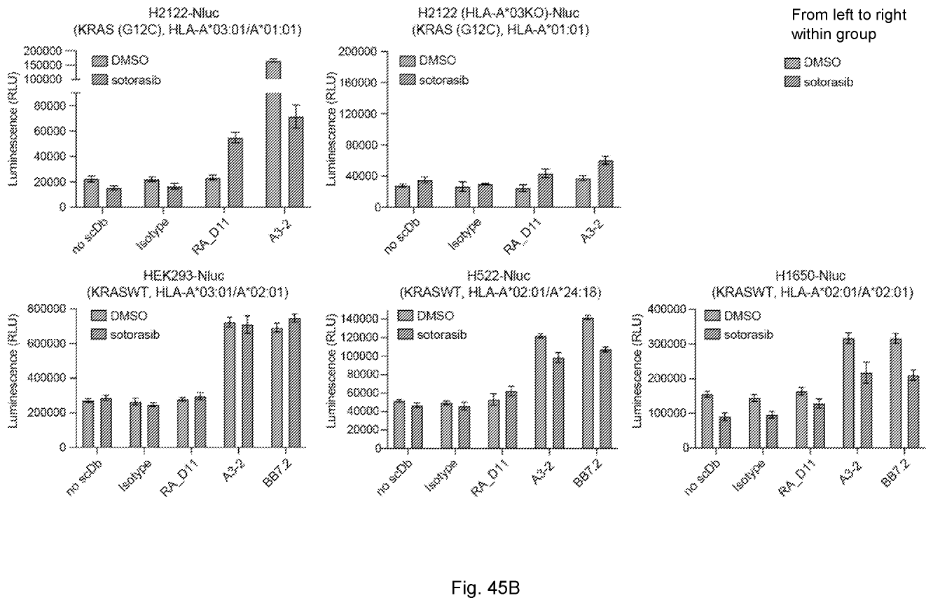

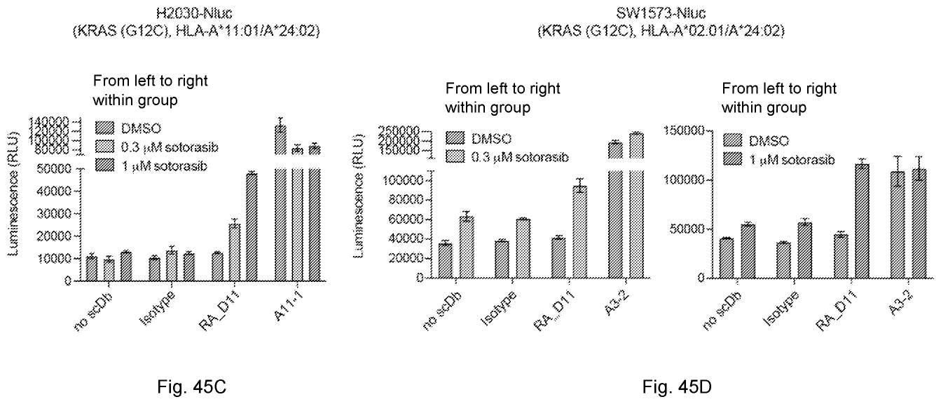

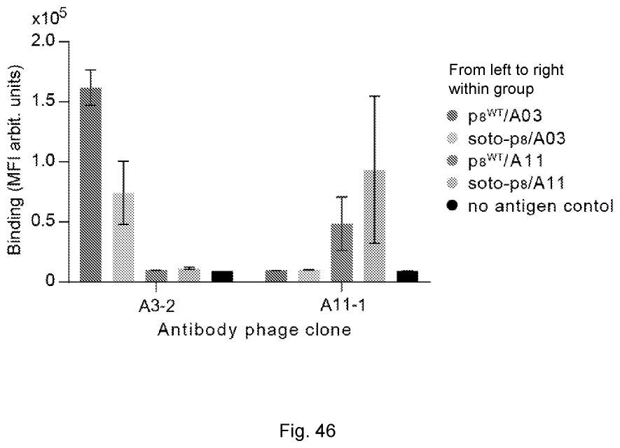

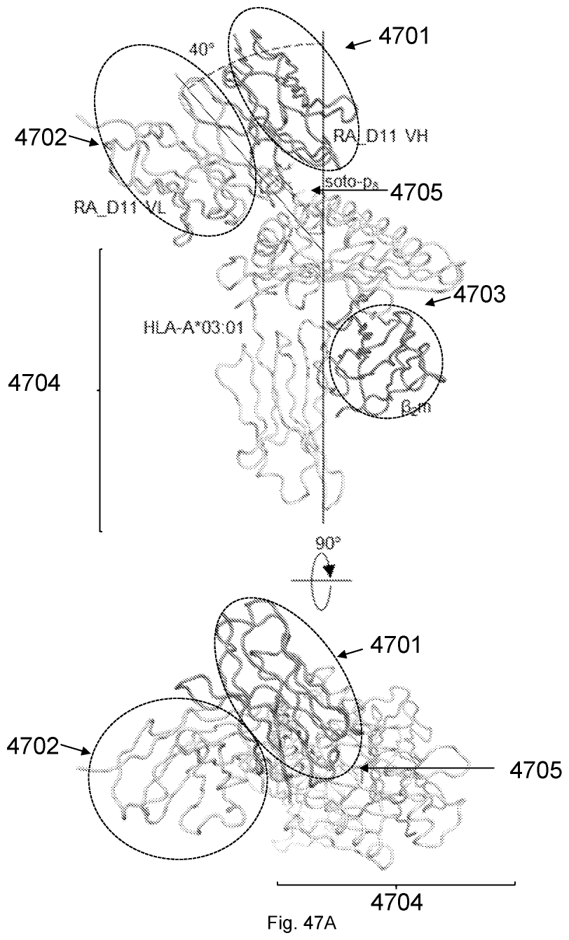

H2122-Nluc cells were cultured in the presence of sotorasib for a week to allow adequate time for the processes of sotorasib engagement with KRAS(G12C), degradation of the sotorasib-KRAS(G12C) conjugate, and loading of the conjugates on HLA to reach a steady state. Remarkably, coculture of sotorasib pre-treated H2122-Nluc cells with T cells in the presence of sotorasib and the RA_D11 scDb resulted in efficient cell killing (FIG. 43C, FIG. 45). Although the copy number of sotorasib-RASG12C peptide conjugate in complex with HLA-A*03 on the cell surface is predicted to be low, the killing efficiency of the sotorasib/scDb combination was comparable to that evoked by a positive control scDb made with an antibody targeting all cell surface-expressed HLA-A*03, irrespective of its bound peptides, clone A3-2 (FIG. 43C, FIG. 46). The EC₅₀ of the RA_D11 scDb on sotorasib-treated H2122-Nluc was 29 pM (FIG. 43D), whereas it showed no killing of vehicle-treated H2122-Nluc cells. Cell killing was dependent on sotorasib concentration, as expected (FIG. 43E). These results support the notion that selective targeting of inhibitor-peptide/MHC complexes could lead to a new immunotherapeutic approach.

A series of rigorous control experiments was performed to validate the proposed mechanism of tumor cell killing. Deletion of the HLA-A3 allele in H2122-Nluc by means of CRISPR/Cas9 technology (FIG. 44B) rendered these cells resistant to killing by the sotorasib/RA_D11 scDb combination (FIG. 43F, FIG. 45). Likewise, the RA_D11 scDb had no cytotoxic effects on cells harboring KRAS (WT) either with matched or mismatched HLAs (FIG. 43F). Hence, killing of H2122 cells by RA_D11 scDb depends on the presence of the covalent targeted therapy drug, its target, and an appropriately matched HLA, providing strong support for the HapImmune™ concept.

Intriguingly, the RA_D11 scDb also killed sotorasib-treated H2030-Nluc (expressing HLA-A11), and SW1573-Nluc (expressing HLA-A02), as anticipated from the binding profile of RA_D11 Fab to purified soto-p/MHC complexes (FIG. 43G-H, FIG. 45). These results demonstrate the potential of the HapImmune™ approach.

In contrast to other antibodies in the art that bind to inhibitor-peptide conjugates, these results demonstrate that the binding partners, compositions, and methods described herein can be used to target a cell surface inhibitor/peptide/MHC complex antigen in the presence of the free inhibitor at a therapeutically relevant concentration. In particular, as shown in FIG. 43, the described scDb effectively killed H2122 cells in the presence of 1 μM sotorasib. Thus, the described approach can be generalized to other covalent drug-peptide complexes.

Further, in the Codebreak-200 trial (NCT04303780), a randomized trial for second line disease comparing sotorasib with the standard of care-docetaxel, progression-free survival was improved by one month, but there was no impact on overall survival. In addition, 10% of patients had grade 3/4 liver toxicity, forcing six patients to be removed from the study, while two patients experienced drug-induced liver injury. However, this toxicity was mitigated by dose reduction, which may have impacted overall survival. The results disclosed herein indicate that these limitations of sotorasib may be mitigated by lowering its dose combined with a binding partner described herein.

### EXAMPLE 10

Cryo-Electron Microscopic Structures of HapImmune™ Antibody in Complex with Sotorasib-Peptide: MHCs and Consensus Sequences of RA_D11 VL and VH Domains

In this example, the cryo-EM structures of a HapImmune™ antibody, RA_D11, in complex with human MHCs is disclosed. HapImmune™ antibodies can bind to drug-peptide conjugate/MHC complexes but do not to the free drugs. This antibody can bind to human MHCs (HLAs), HLA-A*03:01 or HLA-A*11:01, presenting 9mer and 10mer peptides derived from KRAS(G12C) conjugated with a covalent inhibitor, sotorasib. The antibody's functional properties are described in the present application and also published in Hattori, Maso, et al. (PMID 36250888).

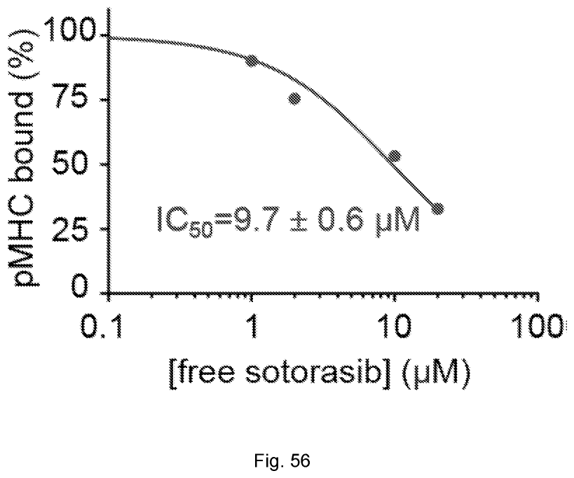

Fab RA_D11 can specifically bind with high affinity to all the four soto-pMCH targets, and it is minimally inhibited by the free inhibitor. Fab RA_D11 selectively binds to soto-pMHCs (FIG. 35, black lines). The minimal inhibition of RA_D11 binding by free sotorasib is a fundamental prerequisite for coadministration with the inhibitor (FIG. 56). RA_D11-based bispecific T cell engager selectively and potently kills sotorasib-resistant lung cancer cells upon sotorasib treatment, only in cells having both the KRAS G12C mutation and the matching HLA (A03 and A11). RA_D11-based scDb induces extremely selective T cell mediated cytotoxicity, being harmless to cells lacking either KRAS(G12C) or the matching HLA (or both) resulting in no off-target toxicity (FIG. 43F).

RA_D11 is a representative of the first set of antibodies that selectively recognize sotorasib-peptide: MHC complexes. Importantly, RA_D11 binds to multiple complexes. It was unclear how the RA_D11 antibody can bind tightly to the at least three peptides presented by three MHCs, while maintaining selectivity toward sotorasib-peptide: MHC complexes over the free drug. Therefore, the cryo-EM structures of the RA_D11 in complex with multiple sotorasib-peptide: MHC complexes were determined. The structures reveal an unusual mode of recognition of the sotorasib-peptide: MHC complexes by the antibody and rationalize how a single antibody can recognize multiple sotorasib-peptide: MHC complexes with high affinity and high selectivity. These structures can greatly help develop next-generation HapImmune™ antibodies.

### Nomenclatures and Amino Acid Sequences of Peptides and Proteins

Peptides Derived from KRAS(G12C)

p₈:
(SEQ ID NO:2)
H2N-VVGACGVGK-OH
p₇:
(SEQ ID NO:2)
H2N-VVVGACGVGK-OH

### HLA Proteins

HLA-A*03:01 heavy chain
(SEQ ID NO:3)
MASG₁SHSMRYFFTSVSRPGRGEPRFIAVGYVDDTQFVRFDSDAASQRME
PRAPWIEQEGPEYWDQETRNVKAQSQTDRVDLGTLRGYYNQSEAGSHTIQ
IMYGCDVGSDGRFLRGYRQDAYDGKDYIALNEDLRSWTAADMAAQITKRK
WEAAHEAEQLRAYLDGTCVEWLRRYLENGKETLQRTDPPKTHMTHHPISD
HEATLRCWALGFYPAEITLTWQRDGEDQTQDTELVETRPAGDGTFQKWAA
VVVPSGEEQRYTCHVQHEGLPKPLTLRWE

(G₁ indicates Gly 1 according to the residue numbering of HLAs. The three residues preceding it originate from expression artifacts.)

HLA-A*11:01 heavy chain
(SEQ ID NO:4)
MASG1SHSMRYFYTSVSRPGRGEPRFIAVGYVDDTQFVRFDSDAASQRME
PRAPWIEQEGPEYWDQETRNVKAQSQTDRVDLGTLRGYYNQSEDGSHTIQ
IMYGCDVGPDGRFLRGYRQDAYDGKDYIALNEDLRSWTAADMAAQITKRK
WEAAHAAEQQRAYLEGRCVEWLRRYLENGKETLQRTDPPKTHMTHHPISD
HEATLRCWALGFYPAEITLTWQRDGEDQTQDTELVETRPAGDGTFQKWAA
VVVPSGEEQRYTCHVQHEGLPKPLTLRWE
β₂microglobulin (β₂m)
GSIQRTPKIQVYSRHPAENGKSNFLNCYVSGFHPSDIEVDLLKNGERIEK
VEHSDLSFSKDWSFYLLYYTEFTPTEKDEYACRVNHVTLSQPKIVKWDRD
M
Fab RA_D11
Heavy chain
(SEQ ID NO:5)
EVQLVESGGGLVQPGGSLRLSCAASGFTFSDYSIHWVRQAPGKGLEWVAS
ISSSSGSTSYADSVKGRFTISADTSKNTAYLQMNSLRAEDTAVYYCARGG
WIAAMDYWGQGTLVTVFNQIKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHT
Light chain
(SEQ ID NO:6)
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTVAAPSVFIFPPSDSQLKSGTASVVCLLNNFYPREAKVQW
KVDNALQSGNSQESVTEQDSKDSTYSLSSTLTLSKADYEKHKVYACEVTH
QGLSSPVTKSFNRGEC

### Results

HLA proteins presenting sotorasib-conjugated peptides derived from KRAS(G12C) were reconstituted, namely, soto-p₈/A03, soto-p₇/A03 and soto-p₇/A11, as described previously in Hattori, Maso, et al. (PMID 36250888). An elbow-rigidifying mutations in the heavy chain of Fab RA_D11 was introduced, Fab RA_D11 was expressed in Escherichia coli and purified. The three peptide/HLA-Fab complexes (soto-p₈/A03-RA_D11, soto-p₇/A03-RA_D11, and soto-p₇/A11-RA_D11) were then formed and purified.

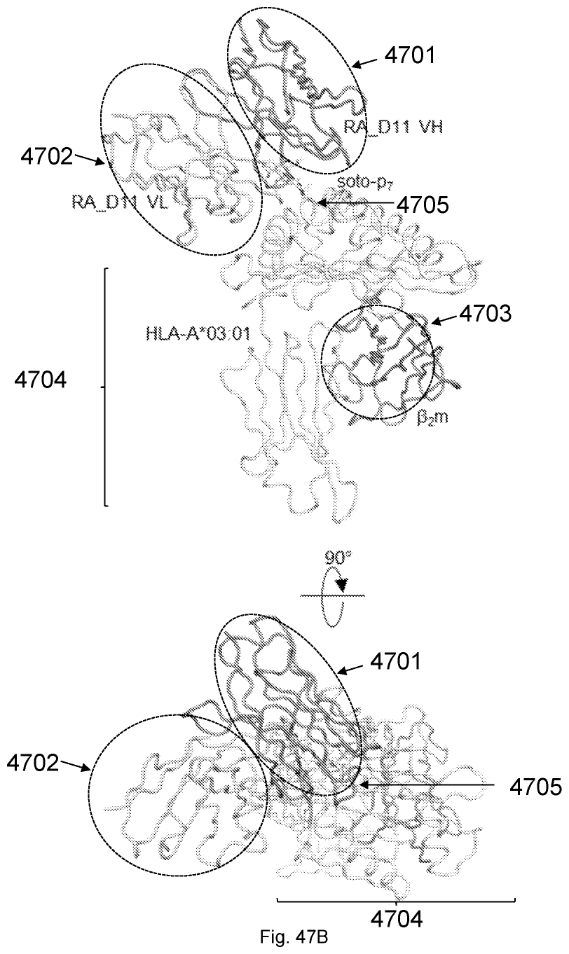

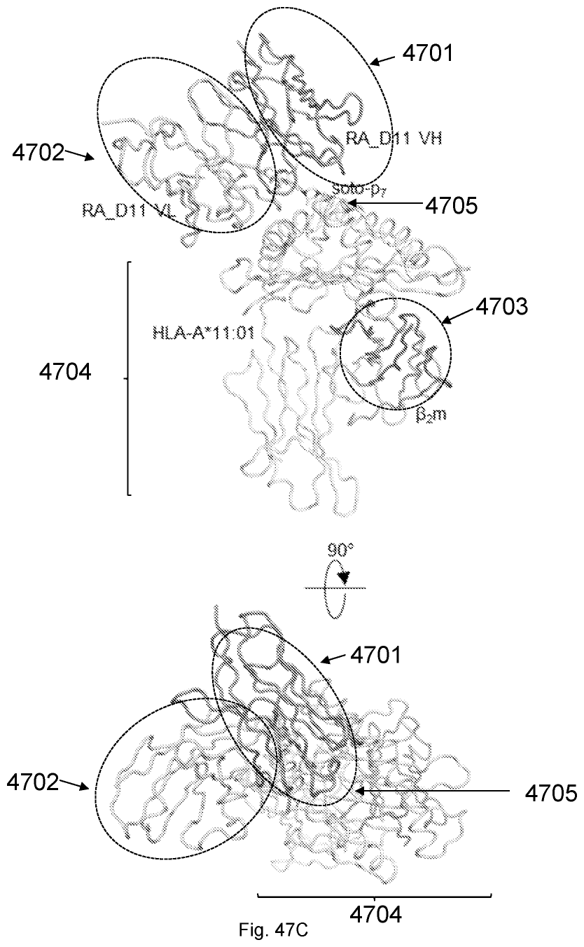

Using single particle cryo-electron microscopy, the molecular structures of the three complexes was determined (FIG. 47). Density maps with nominal resolutions ranging from 3.08 to 3.23 Å were obtained. Although the elbow-rigidifying mutations were utilized, the constant domains of Fab RA_D11 was less-well defined. The constant domain was excluded from model building. Thus, the final structures include the variable domains of Fab RA_D11 and the entire structure of sotorasib-peptide/HLA (FIG. 47).

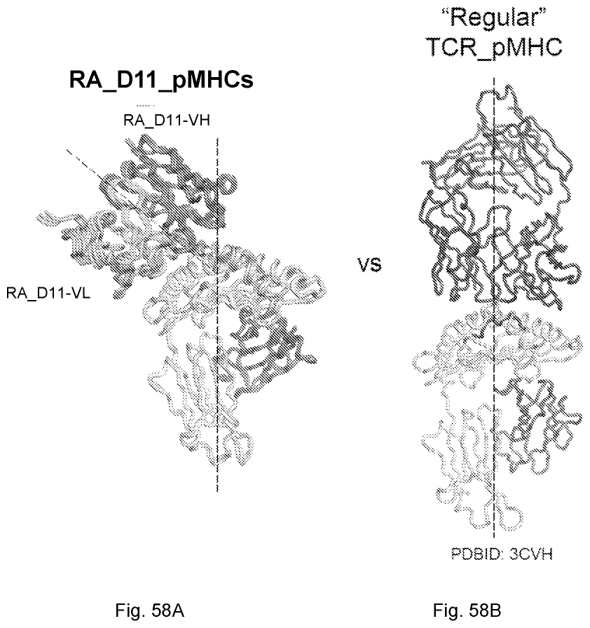

CryoEM structures of RA_D11 bound to multiple drug-peptide/MHC complexes elucidate the molecular basis of target recognition. RA_D11 recognizes soto-p/A03-A11 targets in a similar way, mainly interacting with the HLA surface and sotorasib (with a pocket formed by the interface of the Fab variable chains), while having minimal contacts with the rest of the peptide (p₇ or p₈, FIG. 57A-C). RA_D11 binds to the soto-pMHCs in a non-canonical manner, differently from typical TCR-pMHC engagement (FIGS. 58A and 58B).

Unlike typical T-cell receptors binding to peptide MHC complexes, Fab RA_D11 does not bind to the target pMHC with a head-to-head coaxial interaction. Instead, Fab RA_D11 engages the sotorasib-p/MHC complexes with an angle of about 40° between the axis of the MHC and its own axis (FIG. 47A).

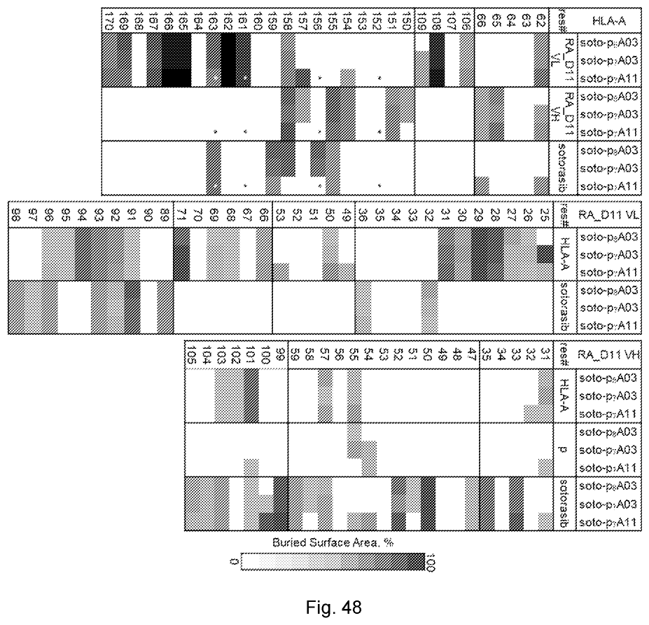

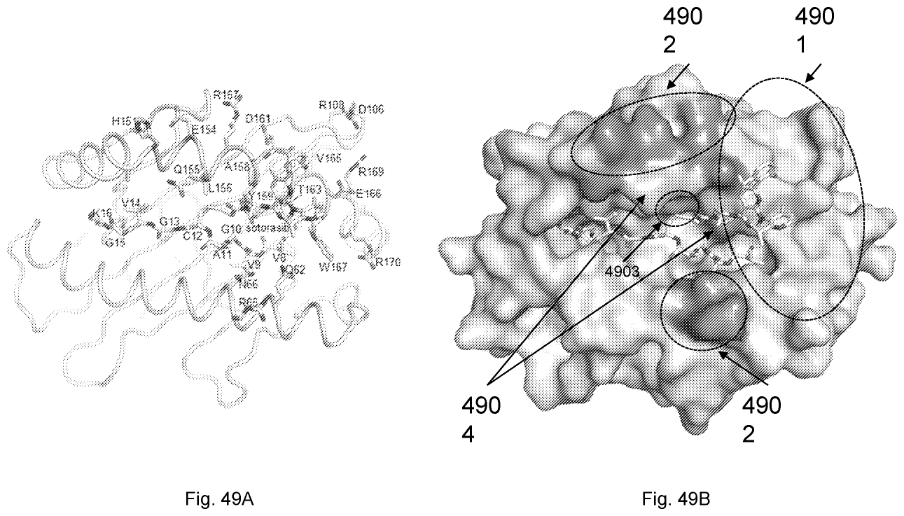

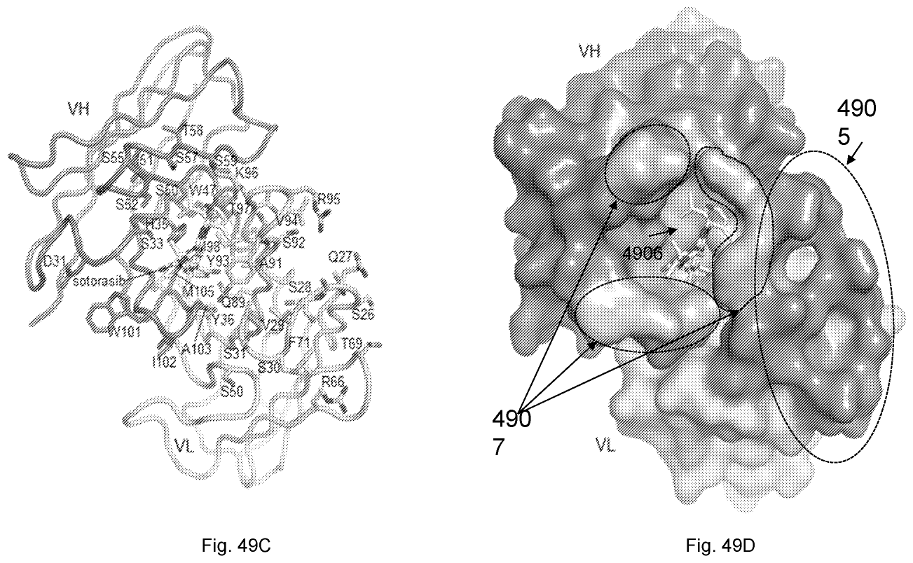

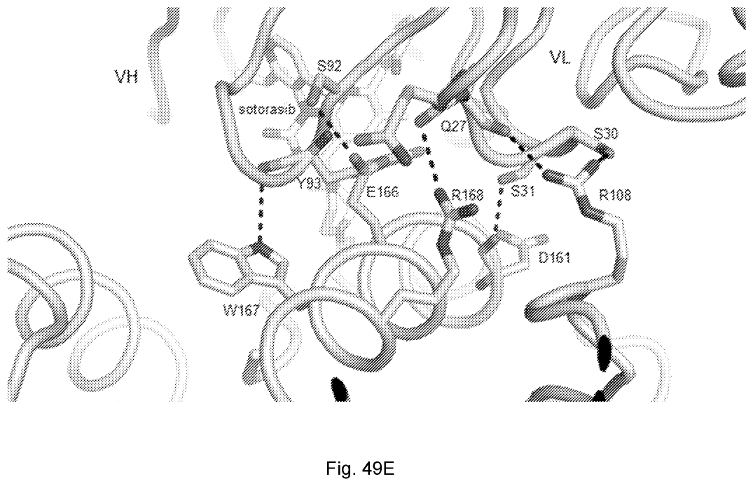

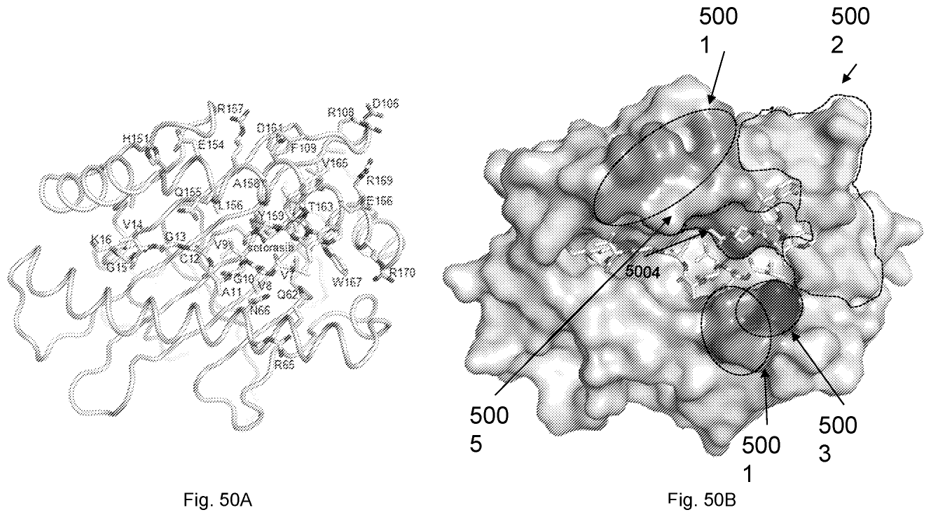

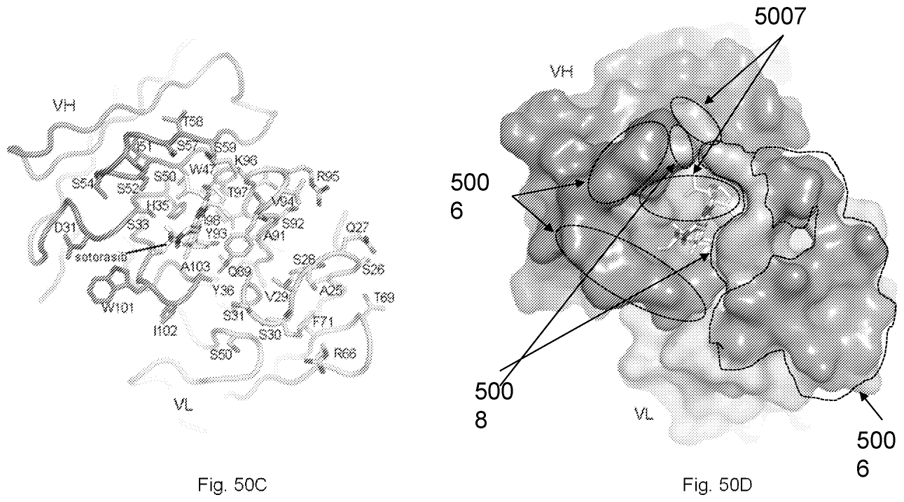

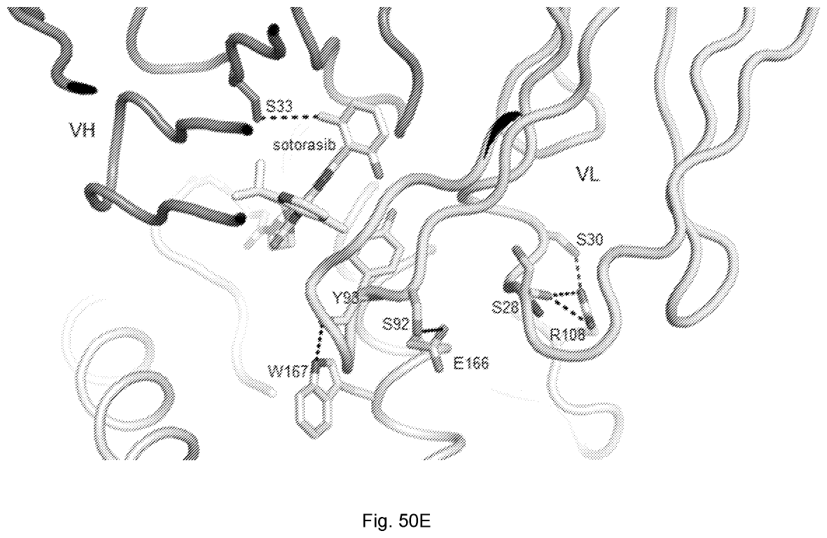

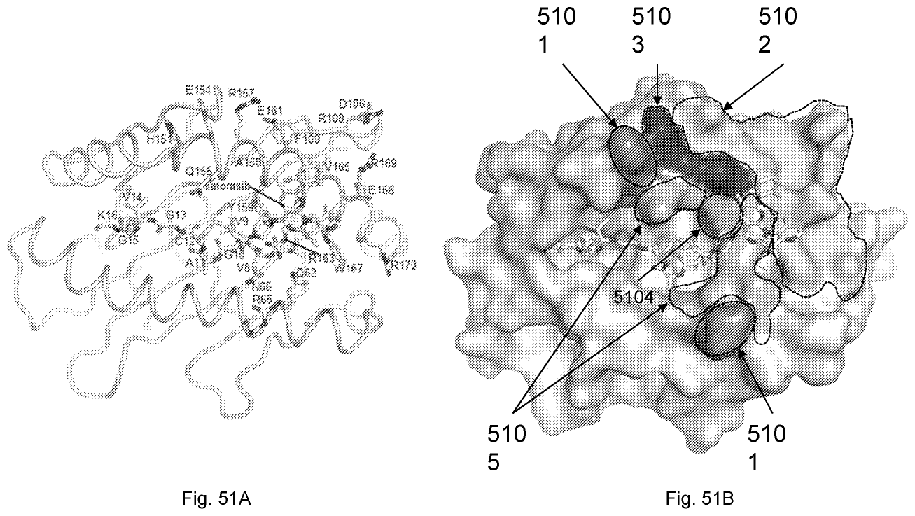

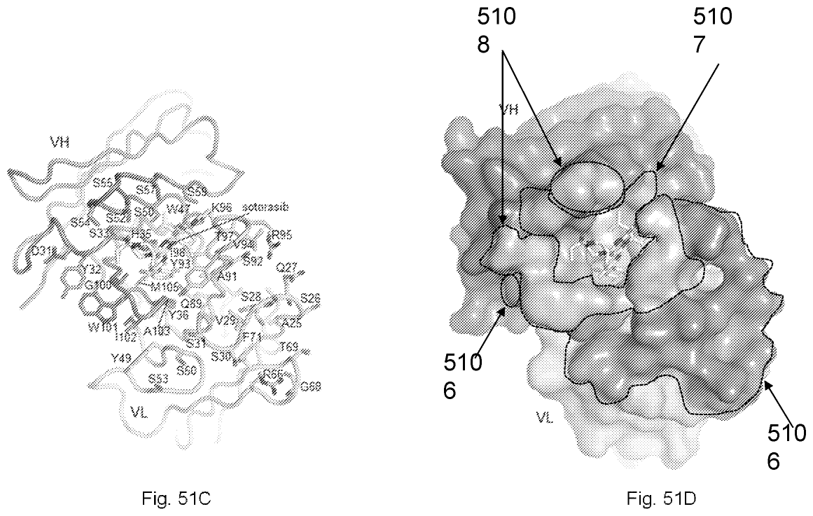

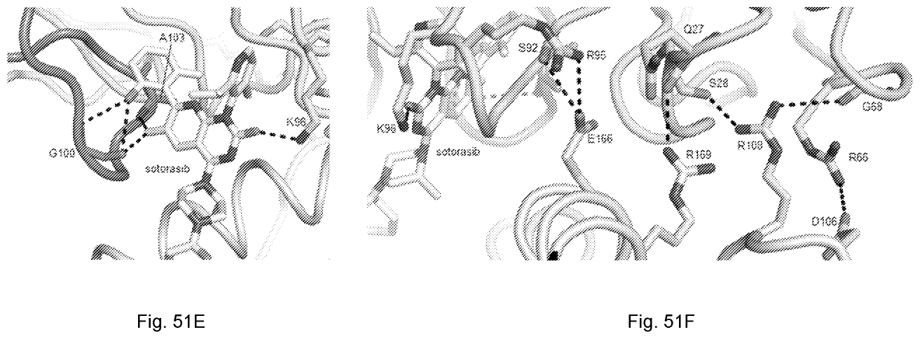

Fab RA_D11, in all the three complexes, mainly interacts with the surface of the HLAs and the sotorasib moiety of the hapten-peptides, forming almost no contacts with the rest of the hapten-peptide (FIG. 47 and FIG. 48). In all the three structures sotorasib was conjugated to Cys12 of KRAS(G12C) peptides and protrudes away from the HLA groove (FIG. 47, FIG. 49B, FIG. 50B, FIG. 51B). The sotorasib moiety is bound to a pocket formed by the interface between the VH and VL domains of Fab RA_D11 (FIG. 47, FIG. 49D, FIG. 50D, FIG. 51D), a feature commonly found in hapten-binding antibodies.

Fab RA_D11 contacts soto-p₈/A03 with an interface area of 1087 Å², interacting mainly the HLA-A*03:01 and sotorasib (RA_D11-HLA: 619 Å²; RA_D11-sotorasib: 452 Å²; RA_D11-p₈: 35 Å²), with a favorable calculated solvation free energy gain upon formation of the interface estimated using the PISA server (FIG. 48, FIG. 49A-D). A total of 20 residues of the HLA-A*03:01 are involved in the interaction with Fab RA_D11, and a total of 39 residues of Fab RA_D11 make contact with soto-p₈/A03 (VL: 22; VH: 17) (FIGS. 48 and 49). There are 6 hydrogen bonds between HLA-A*03:01 and the VL domain (FIG. 49E and Table L).

Although the p₇ peptide is longer by one residue than the p₈ peptide, the overall structures of the two complexes, with soto-p₈/A03 and soto-p₇/A03, resemble each other closely (FIG. 47). In the complex with soto-p₇/A03, the antibody has an interface area of 1106 Å², again mainly interacting with the HLA-A*03:01 and sotorasib but not with the peptide (RA_D11-HLA: 627 Å²; RA_D11-sotorasib: 455 Å²; RA_D11-p₈: 5 Å²), with a favorable calculated solvation free energy gain upon formation of the interaction interface (FIG. 50A-D and Table M). A total of 22 residues of the HLA-A*03:01 are involved in the interaction with Fab RA_D11, whereas a total of 45 residues of the antibody are involved in the interface with soto-p₇/A03 (VL: 23; VH: 22) (FIG. 48 and FIG. 50). Fab RA_D11 forms 5 hydrogen-bonds with HLA-A*03:01 through its VL, and one hydrogen-bond between its VH and sotorasib (FIG. 50E and Table L).

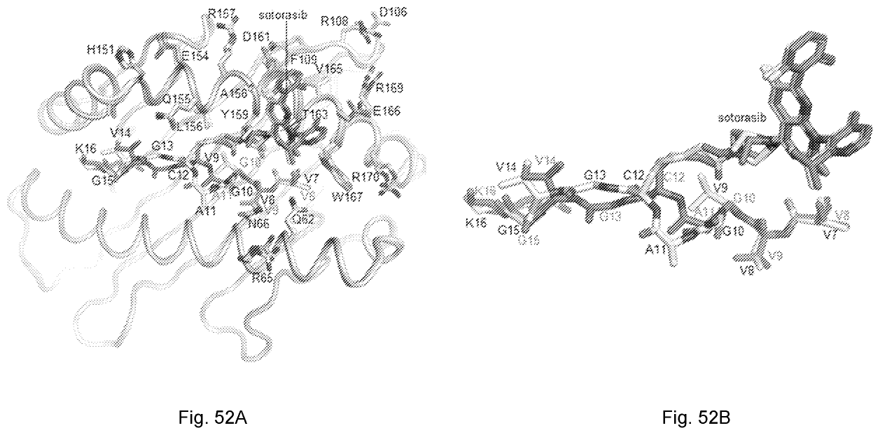

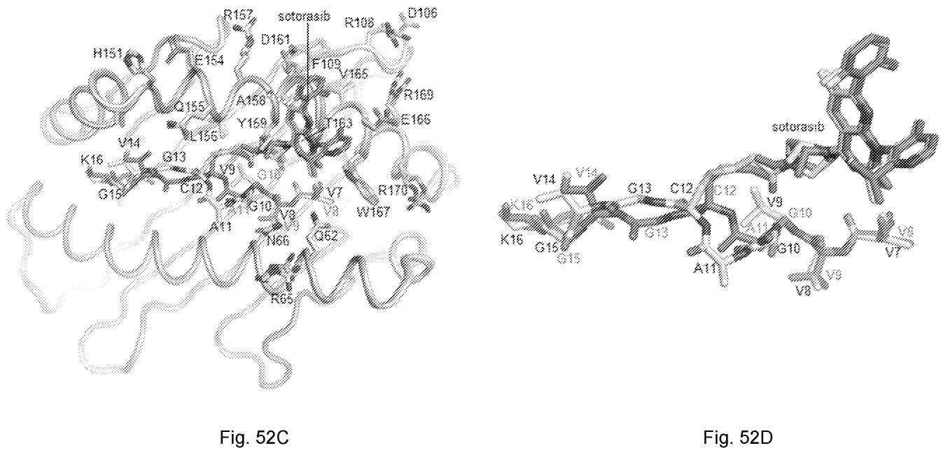

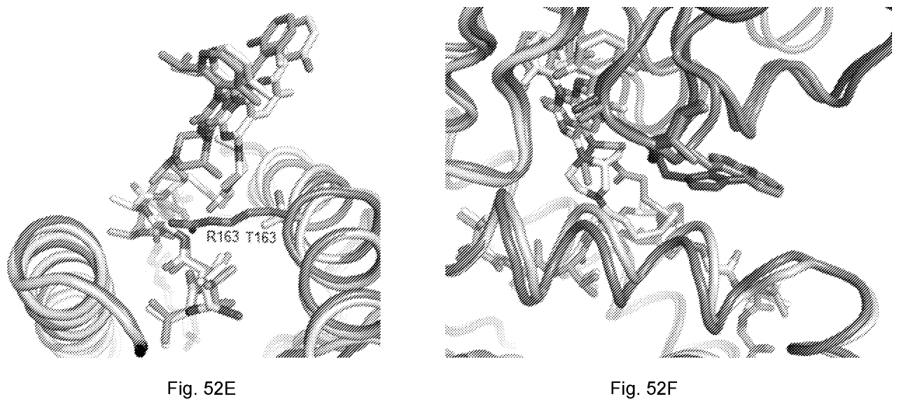

In the structures with soto-p₈/A03 and soto-p₇/A03, HLA-A*03 and sotorasib are almost perfectly superimposable (FIGS. 52A and 52B). The main difference between the two structures is the conformations of the peptide portion of the soto-p₈ and soto-p₇ conjugates, with the former being stretched along the HLA pocket, whereas soto-p₇ takes on a curved conformation to accommodate one extra residue (FIGS. 52A and 52B).

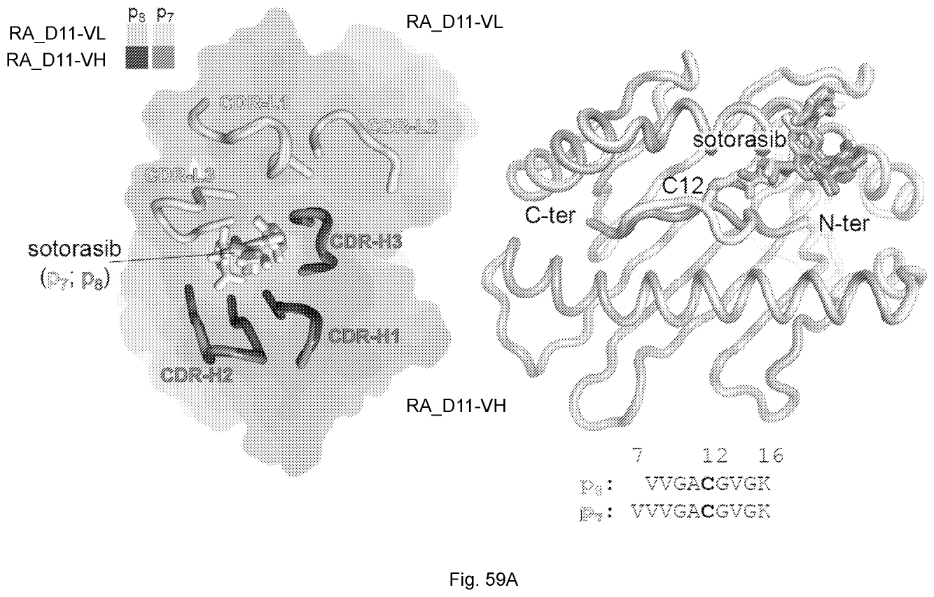

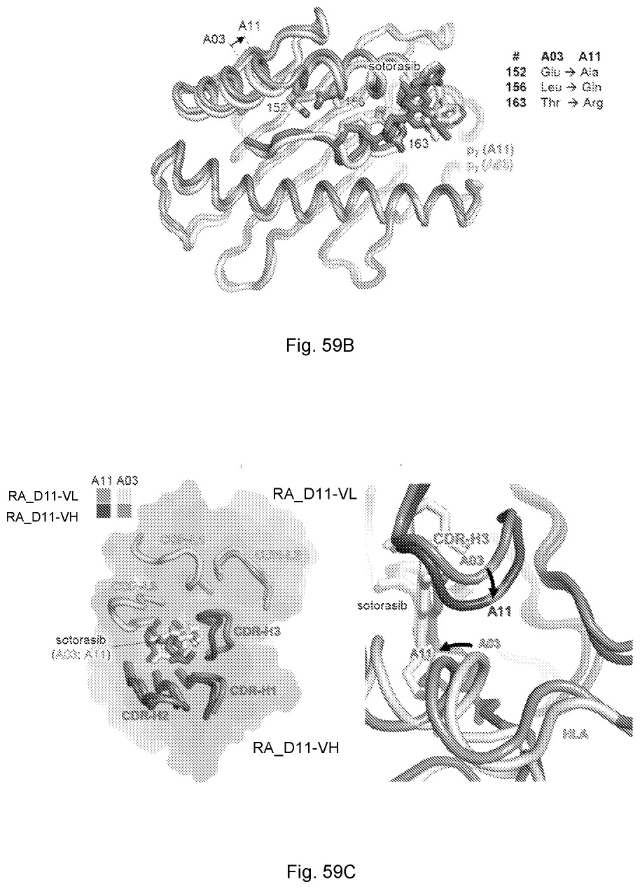

Fab RA_D11 can recognize both soto-p₇/A03 and soto-p₈/A03 in an almost identical way thanks to a different peptide accommodation of soto-p₈ (stretched) vs. soto-p₇ (bent) into the HLA-A*03 pocket. When soto-p₇ peptide is loaded by HLA-A*03 or A*11 different residues between the two HLAs result in a slightly different conformation of the Fab CDRs (in particular CDR-H3) when binding to the targets (FIG. 59A-C).

HLA-A*03 and HLA-A*11 belong to the same HLA superfamily and they are highly homologous, with a total of 7 different residues (residues 9, 90, 105, 152, 156, 161 and 163). Consistent with the observation that RA_D11 exhibits similar affinity to these antigens, the overall structure of the complexes of RA_D11 with soto-p₇/A03 and soto-p₇/A11 are similar (FIG. 47). The interaction between RA_D11 and soto-p₇/A11 involves an interface area of 1255 Å². Fab RA_D11 contacts the target soto-p/MHC mainly interacting with the HLA-A*11:01 and sotorasib (RA_D11-HLA: 751 Å²; RA_D11-sotorasib: 496 Å²; RA_D11-p₈: 8 Å²), with a favorable calculated solvation free energy gain upon formation of the interaction interface (FIGS. 51A-D and Table M). A total of 20 residues of the HLA-A*11:01 and a total of 44 residues of the antibody (VL: 25; VH: 19) are involved in the interaction (FIG. 48 and FIGS. 51A and 51C). Fab RA_D11 also forms 6 hydrogen-bonds with HLA-A*11:01 through its VL, and 5 hydrogen-bonds with sotorasib (one through its VH and the rest through its VL) (FIGS. 51E and 51F and Table M).

RA_D11 accommodates just one residue (R163) that is different between HLA-A*03 and A*11 in the interface. Three additional residues (A152, Q156 and E161) located in the α2 domain are different between HLA-A*03 and A*11 and are adjacent to the Fab-HLA interface (FIG. 48, FIG. 51A, and FIGS. 52C-E). The presence of R163 in HLA-A*11 instead of T163 in HLA-A*03 can result in distinct modes of presentation of soto-p₇ hapten-peptide.

The conformation of soto-p₇ in the two complexes exhibits differences, in particular in the positions of A11 and sotorasib (FIG. 52C-F). Moreover, residues A152 and Q156, located in the peptide-presenting groove, are different between HLA-A*11 and A*03 (the latter having E152 and L156 instead), resulting in a contracted pocket in HLA-A*11 compared with that of A*03 when presenting the same soto-p₇ peptide (FIG. 52C). These differences in the conformation of HLA and soto-p₇ result in a different conformation of Fab RA_D11 in the two complexes. W101 in CDRH3 of Fab RA_D11 shows a clear shift between the two structures (FIG. 52F). A mutation, Pro106 to Ala, in CDRH3 was introduced during affinity maturation of AMRA3-7D that increased the affinity to soto-p₇/A11. This mutation may have increased the range of CDRH3 conformation, which in turn allowed the movement of W101.

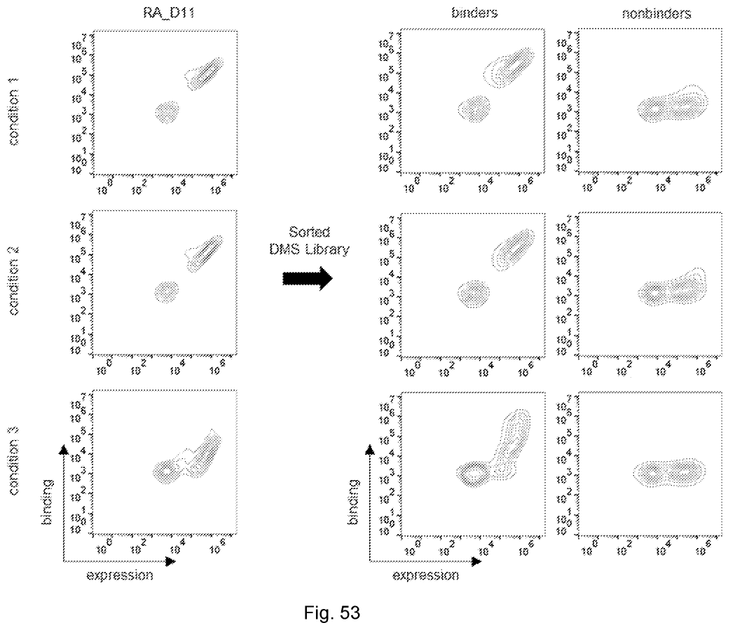

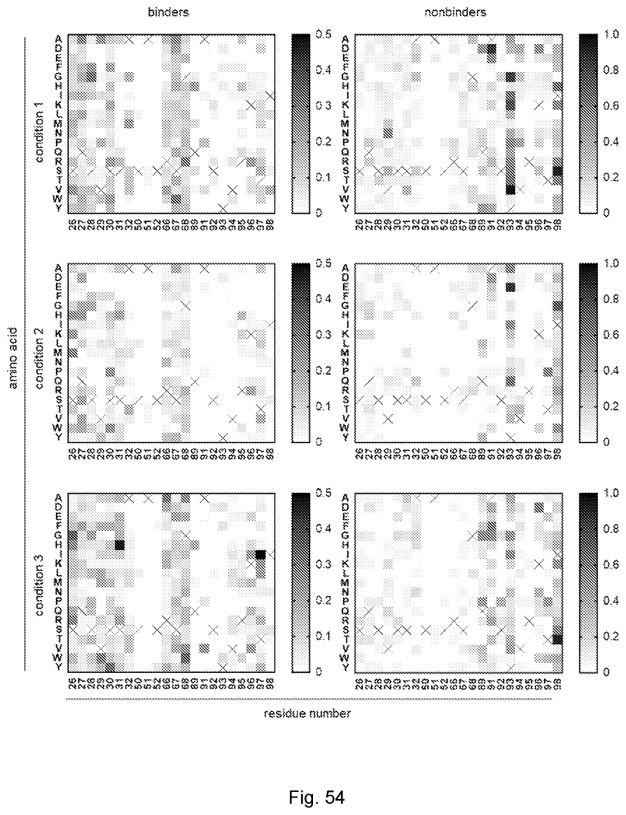

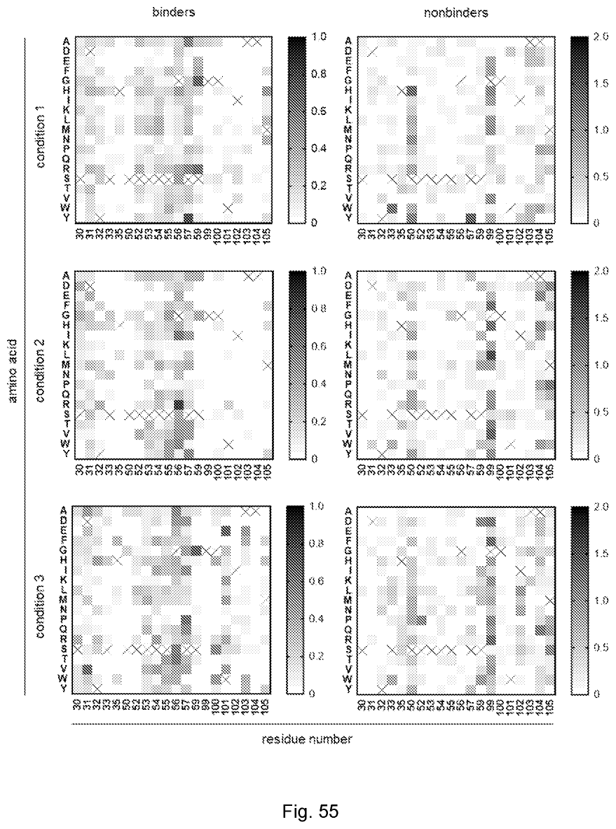

Deep mutational scanning of residues of clone RA_D11 that are located proximal to sotorasib, peptide, or HLA in the structures (selecting from positions that are within 5 Å of any atom of the sotorasib-peptide conjugate or of the HLAs) was performed. In total, 22 positions in VL and 20 positions in VH were analyzed. Single mutations that retain or lose binding to the antigen (Condition 1:10 nM soto-p₇/HLA-A03; condition 2:10 nM soto-p7/HLA-A11; condition 3:10 nM soto-p₈/A02 tetramerized using streptavidin-DyLight650) were recovered and their identities were deduced using deep sequencing. The analysis identified permissible and non-permissible amino acids at each position (FIG. 53-FIG. 55), from which consensus sequences were derived (Tables N and O).

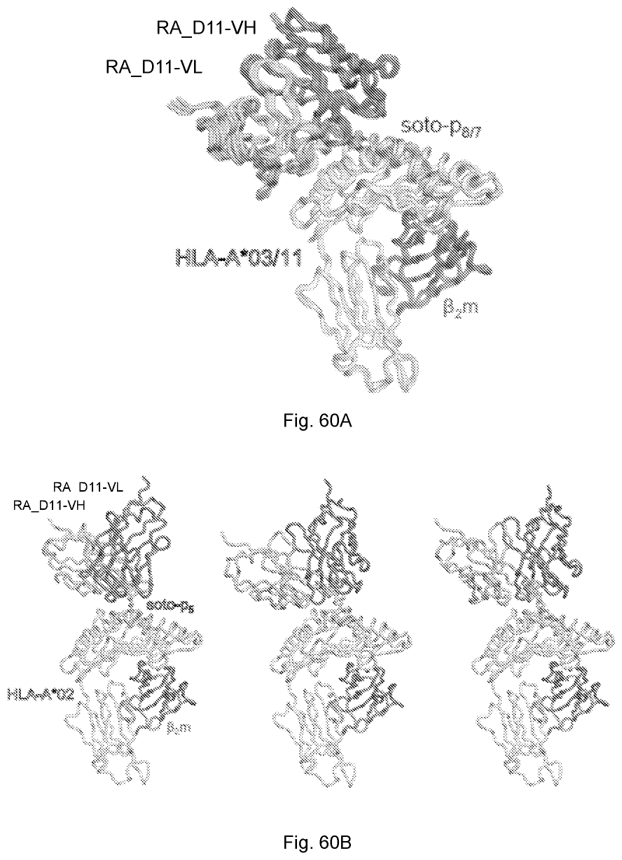

RA_D11 can also bind to soto-p₈/A02, and accordingly RA_D11-based specific T-cell engager can selectively kill KRAS(G12C) cancer cells having the most diffused HLA-A*02 as well (FIG. 36). RA_D11 binds soto-p₈/A02 in a non-univocal way, showing multiple possible orientations and have a reduced surface contract with the HLA with respect to soto-p/A03-A11 targets binding (FIGS. 60A and 60B).

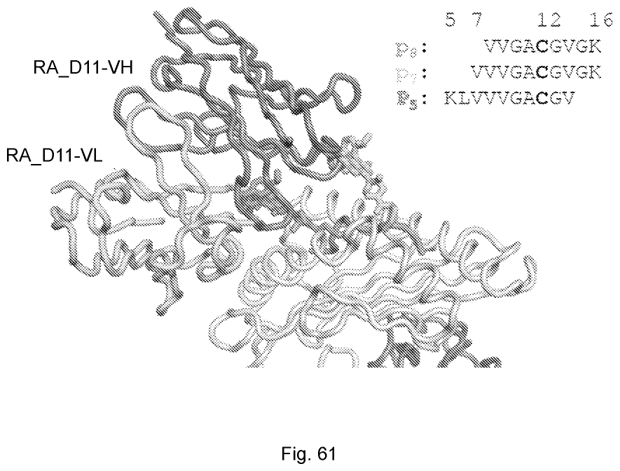

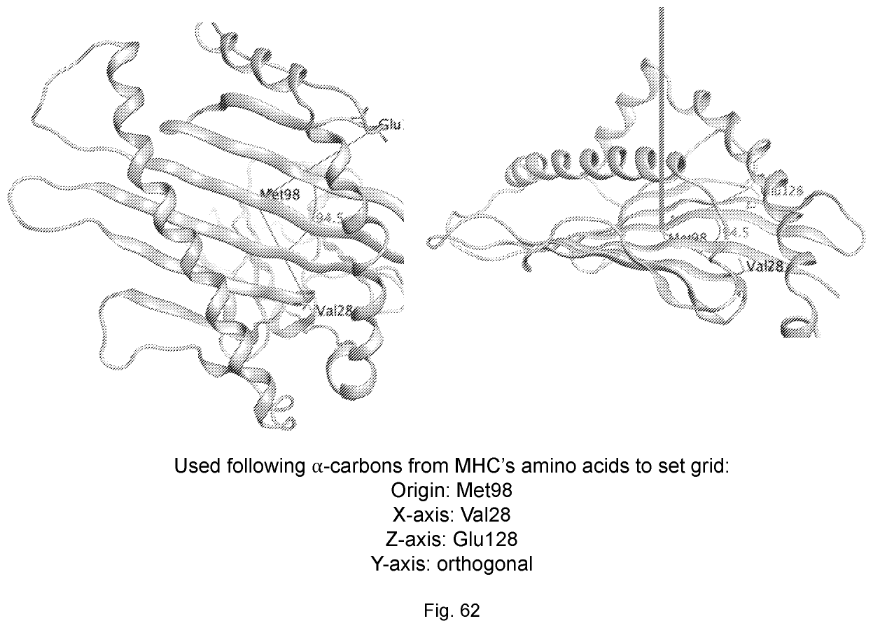

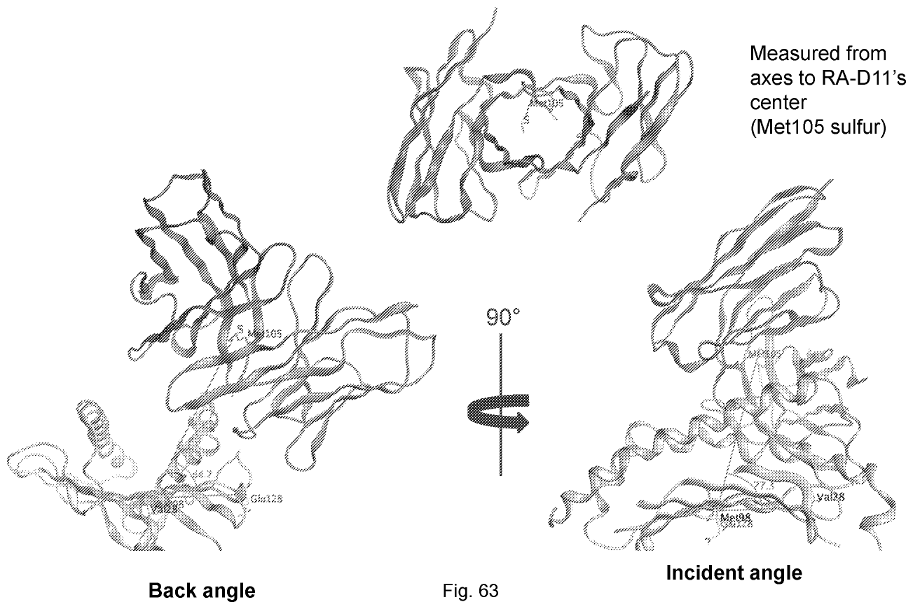

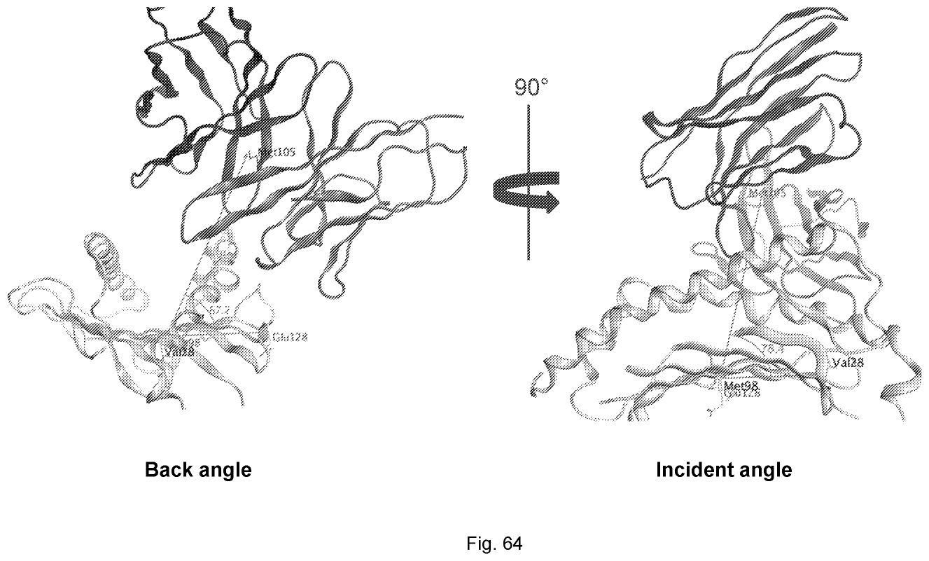

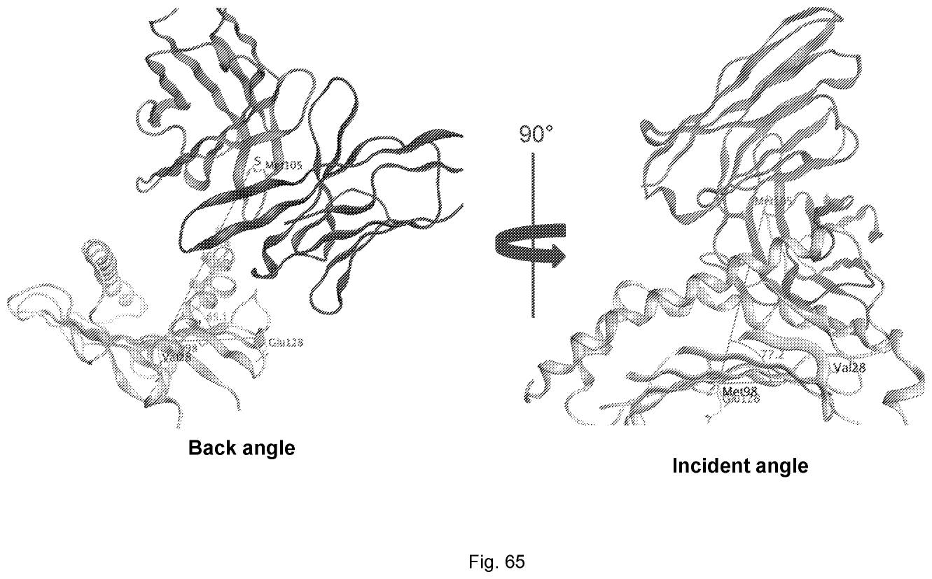

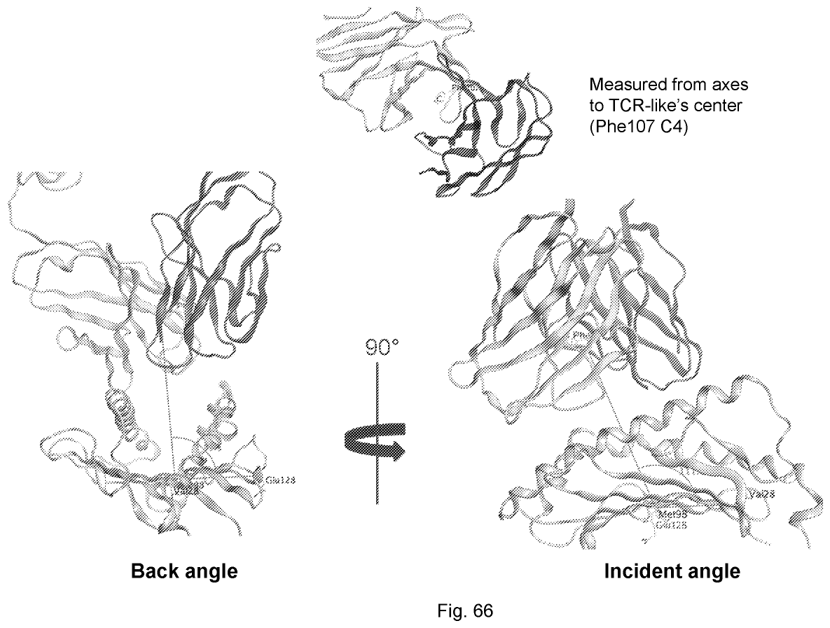

Soto-p₅ is a much different peptide from soto-p₇₋₈ and must be presented in a distinct way (FIG. 61).

TABLE L
Hydrogen-bonds formed between Fab RA_D11 and soto-pMHCs
in the three complexes, as identified with the PISA server.

| | soto-pMHC | | H-bond | Fab RA_D11 | |
|---|---|---|---|---|---|
| | residue (molecule) | atom | length, Å | residue (chain) | atom |
| soto-p₈/ | R108 (HLA-A*03:01) | NH1 | 3.0 | S28 (VL) | O |
| A03_RA_D11 | R108 (HLA-A*03:01) | NH2 | 2.8 | S30 (VL) | OG |
| | W167 (HLA-A*03:01) | NE1 | 3.8 | Y93 (VL) | O |
| | R169 (HLA-A*03:01) | NH1 | 3.2 | Q27 (VL) | O |
| | D161 (HLA-A*03:01) | O | 2.9 | S31 (VL) | OG |
| | E166 (HLA-A*03:01) | OE2 | 3.2 | S92 (VL) | OG |
| soto-p₇/ | R108 (HLA-A*03:01) | NH1 | 3.4 | S28 (VL) | O |
| A03_RA_D11 | R108 (HLA-A*03:01) | NH2 | 2.8 | S30 (VL) | OG |
| | R108 (HLA-A*03:01) | NH2 | 3.4 | S28 (VL) | O |
| | W167 (HLA-A*03:01) | NE1 | 3.8 | Y93 (VL) | O |
| | E166 (HLA-A*03:01) | OE1 | 3.8 | S92 (VL) | OG |
| | MOV (soto-p₇) | F2 | 3.6 | S33 (VH) | OG |
| soto-p₇/ | R169 (HLA-A*11:01) | NH1 | 3.2 | Q27 (VL) | O |
| A11_RA_D11 | R108 (HLA-A*11:01) | NH2 | 2.8 | S28 (VL) | O |
| | R108 (HLA-A*11:01) | NH1 | 3.5 | G68 (VL) | O |
| | D106 (HLA-A*11:01) | OD2 | 3.7 | R66 (VL) | NH2 |
| | E166 (HLA-A*11:01) | OE2 | 3.4 | S92 (VL) | OG |
| | E166 (HLA-A*11:01) | OE2 | 3.5 | R95 (VL) | NH1 |
| | MOV (soto-p₇) | O1 | 2.6 | K96 (VL) | NZ |
| | MOV (soto-p₇) | F1 | 2.4 | G100 (VH) | O |
| | MOV (soto-p₇) | O3 | 3.3 | G100 (VH) | O |
| | MOV (soto-p₇) | F1 | 3.4 | A103 (VH) | N |
| | MOV (soto-p₇) | O3 | 3.3 | G100 (VH) | N |

TABLE M
Analysis of the Fab RA_D11- soto-pMHCs interaction interfaces.
Interface areas and solvation free energy gain upon formation
of the interface (ΔG, kcal/mol), as calculated by PDBePISA
(https://www.ebi.ac.uk/msd-srv/prot_int/cgi-bin/piserver).
The value is calculated as the difference in total solvation
energies of isolated and interfacing structures. Negative
ΔG corresponds to positive protein affinity. This value
does not include the effect of satisfied hydrogen bonds
and salt bridges across the interface.

| | | | | | Global |
|---|---|---|---|---|---|
| | | | Fab | ΔG | ΔG |
| | soto-pMHC | Interface | RA_D11 | (kcal/ | (kcal/ |
| | molecule | area, Å² | chain | mol) | mol) |
| soto-p₈/ | HLA-A*03:01 | 178.5 | VH | −2.3 | −7.7 |
| A03_RA_D11 | | 448.9 | VL | −0.5 | |
| | sotorasib | 240.9 | VH | −2.1 | |
| | | 213.7 | VL | −2.6 | |
| | p₈ | 5.1 | VH | −0.2 | |
| soto-p₇/ | HLA-A*03:01 | 171.6 | VH | −1.9 | −7.8 |
| A03_RA_D11 | | 447.5 | VL | −0.9 | |
| | sotorasib | 241.2 | VH | −1.6 | |
| | | 210.6 | VL | −2.7 | |
| | p₇ | 35.0 | VH | −0.7 | |
| soto-p₇/ | HLA-A*11:01 | 203.3 | VH | −1.8 | −2.2 |
| A11_RA_D11 | | 548.0 | VL | 1.3 | |
| | sotorasib | 298.2 | VH | −0.5 | |
| | | 197.5 | VL | −1.1 | |
| | p₇ | 7.8 | VH | −0.1 | |

### Methods

pMHCI Preparation

KRAS(G12C) peptides ((H₂N-VVGACGVGK-OH (SEQ ID NO: 1) and H₂N-VVVGACGVGK-OH (SEQ ID NO: 2) were reacted with AMG-510 (Selleckchem) and loaded onto HLA-A*03:01 and HLA-A*11:01, produced in house. The peptide-loaded HLA mixtures were further purified using size-exclusion chromatography with a Superdex S200 column. Details are previously described in Hattori, Maso, et al. (PMID 36250888).

### Antibody Production

The gene encoding RA_D11 antibody was cloned into a Fab expression vector (modified pTac for rigidified heavy-chain hinge) and Fab proteins were produced using the E. coli 55244 bacterial strain (ATCC) and purified using a HiTrap Protein G HP column (Cytiva).

### Fab-p/MHC Complex Preparation

Purified soto-p/MHC samples were incubated with Fab RA_D11 at a 1:1.5 molar ratio (p/MHC:Fab), and the complexes (soto-p₈/A03_RA_D11; soto-p₇/A03_RA_D11; soto-p₇/A11_RA_D11) were purified by size-exclusion chromatography with a Superdex S200 column (Cytiva).

### Cryo-EM Grid Preparation

Fab-pMHCs complexes at a concentration of 1 to 2 mg/ml were frozen in R0.6/1 gold foil on gold 300 mesh Cryo-EM grids (QUANTIFOIL) as follows. Cryo-EM grids were first hydrophilized using a PELCO easiGlow™ Glow Discharge Cleaning System (Ted Pella). (1H, 1H, 2H, 2H-Perfluorooctyl)-β-D-Maltopyranoside (Anatrace) was added to samples at a final concentration of 0.6 mM. Each sample (3 μl) was loaded onto the hydrophilized grids and frozen in liquid ethane using a FEI Vitrobot™ Mark IV (Thermo Scientific).

### Cryo-EM Data Collection and Processing

Cryo-EM data were collected using a Titan-Krios 300 kV TEM (Thermo Fisher scientific) equipped with a Gatan K3 camera. For each sample, 2500 to 5000 images were collected with a 105000× magnification (final pixel size of images: 0.825 Å). Collected data were processed using the CryoSPARC™ software platform. Resulting maps were used to build structure models using WinCoot and Phenix 1.18.2 software. As starting models, PDBID 3RL1 for pMHC moieties and a model of Fab RA_D11 were generated with the fully automated protein structure homology-modelling server SWISS-MODEL (https://swissmodel.expasy.org/). The interaction interfaces in the obtained structures were then analyzed using the PDBePISA tool (https://www.ebi.ac.uk/pdbe/prot_int/pistart.html).

### Deep Mutational Scanning

Deep mutational scanning was performed, following methods previously published in Hattori, Maso, et al. (PMID 36250888), by constructing separate libraries for VL positions and VH positions, selecting from the ones that are within 5 Å of any atom of the sotorasib-peptide conjugate or of the HLAs. Each of the chosen residues of clone RA_D11 was diversified, one amino acid at a time, to all other amino acids except for Cys using oligo pools (Twist Bioscience). The libraries were sorted using soto-p₇/A03 and soto-p₇/A11 at a target concentration of 10 nM, soto-p₅/A02 pre-complexed with streptavidin-DyLight650 (to enhance effective binding via multivalent interaction) at 10 nM. Plasmids containing scFv genes were purified from the enriched pools of yeast cells, using Zymoprep Yeast Plasmid Miniprep II (Zymo Research Corporation), and the scFv genes were amplified and sequenced on a MiSeq sequencer (Illumina). Sequencing data were analyzed using a set of in-house developed UNIX and Python scripts to deduce the number of reads for each mutation.

TABLE N
Permissible mutations include mutations that retain binding to at least
one of the targets tested, i.e., soto-p₇/A03, soto-p₇/A11 and soto-p₅/
A02. Consensus amino acid residue is defined as the parental amino acid
residue plus permissible mutations.
Parental and consensus sequences of RA_D11 VL

| | Parental | | |
|---|---|---|---|
| Position | amino acid | | |
| (VL) | residue | permissible mutation | Consensus amino acid residue |
| 26 | S | A, D, E, F, G, H, I, K, L, | A, D, E, F, G, H, I, K, L, |
| | | M, N, P, Q, R, T, V, W, Y | M, N, P, Q, R, S, T, V, W, Y |
| 27 | Q | A, D, E, F, G, H, I, K, L, | A, D, E, F, G, H, I, K, L, M, |
| | | M, P, R, S, T, V, W, Y | P, Q, R, S, T, V, W, Y |
| 28 | S | E, F, G, H, I, L, M, P, Q, | E, F, G, H, I, L, M, P, Q, R, |
| | | R, T, V, W, Y | S, T, V, W, Y |
| 29 | V | A, E, F, I, L, M, P, R, W, Y | A, E, F, I, L, M, P, R, V, W, Y |
| 30 | S | A, D, F, H, I, K, L, M, P, | A, D, F, H, I, K, L, M, P, Q, |
| | | Q, R, T, V, W, Y | R, S, T, V, W, Y |
| 31 | S | A, E, F, G, H, I, L, N, Q, | A, E, F, G, H, I, L, N, Q, R, |
| | | R, T, V, W, Y | S, T, V, W, Y |
| 32 | A | I, L, M, S, T, V | A, I, L, M, S, T, V |
| 50 | S | F, L, T | F, L, S, T |
| 51 | A | S, T, V | A, S, T, V |
| 52 | S | F, Y | F, S, Y |
| 66 | R | A, D, E, F, H, I, K, L, M, | A, D, E, F, H, I, K, L, M, N, |
| | | N, Q, S, T, V, W, Y | Q, R, S, T, V, W, Y |
| 67 | S | A, D, E, F, G, H, I, K, L, | A, D, E, F, G, H, I, K, L, M, |
| | | M, N, P, Q, R, T, V, W, Y | N, P, Q, R, S, T, V, W, Y |
| 68 | G | A, D, E, F, H, I, K, L, M, | A, D, E, F, G, H, I, K, L, M, |
| | | N, P, Q, R, S, T, V, W, Y | N, P, Q, R, S, T, V, W, Y |
| 89 | Q | G, H, M | G, H, M, Q |
| 90 | Q | | Q |
| 91 | A | P, V | A, P, V |
| 92 | S | D, K, L | D, K, L, S |
| 93 | Y | F | F, Y |
| 94 | V | F, M, Q, R, S, W | F, M, Q, R, S, V, W |
| 95 | R | A, E, F, H, I, K, L, M, N, | A, E, F, H, I, K, L, M, N, |
| | | Q, S, W | Q, R, S, W |
| 96 | K | E, H, I, M, Q, R, T, V, Y | E, H, I, K, M, Q, R, T, V, Y |
| 97 | T | D, I, K, L, M, N, Q, R, S, | D, I, K, L, M, N, Q, R, S, |
| | | V, W, Y | T, V, W, Y |
| 98 | I | L, V | I, L, V |

TABLE O
Permissible mutations include mutations that retain binding to at least
one of the targets tested, i.e., soto-p₇/A03, soto-p₇/A11 and soto-p₅/
A02. Consensus amino acid residue is defined as the parental amino acid
residue plus permissible mutations.
Parental and consensus sequences of RA_D11 VH

| | Parental | | |
|---|---|---|---|
| Position | amino acid | | |
| (VH) | residue | permissible mutation | Consensus amino acid residue |
| 30 | S | A, D, E, F, G, H, I, K, M, | A, D, E, F, G, H, I, K, M, |
| | | N, P, R, Q, T, V, W, Y | N, P, R, S, Q, T, V, W, Y |
| 31 | D | A, E, F, G, H, K, L, M, P, | A, D, E, F, G, H, K, L, M, |
| | | Q, R, S, T, V, W, Y | P, Q, R, S, T, V, W, Y |
| 32 | Y | A, E, F, H, K, M, N, R, S, | A, E, F, H, K, M, N, R, S, |
| | | W | Y, W |
| 33 | S | D, G, H, N, Q | D, G, H, N, Q, S |
| 34 | I | | I |
| 35 | H | F, L, M, W | F, H, L, M, W |
| 50 | S | A, G | A, G, S |
| 51 | I | | I |
| 52 | S | A, F, G, H, I, K, M, R, T, | A, F, G, H, I, K, M, R, S, |
| | | V, Y | T, V, Y |
| 53 | S | A, D, E, F, G, H, I, K, L, | A, D, E, F, G, H, I, K, L, |
| | | M, P, Q, R, T, V, W, Y | M, P, Q, R, S, T, V, W, Y |
| 54 | S | A, D, F, G, H, I, K, L, M, | A, D, F, G, H, I, K, L, M, |
| | | N, Q, R, V, W, Y | N, Q, R, S, V, W, Y |
| 55 | S | A, D, F, G, H, I, M, P, Q, | A, D, F, G, H, I, M, P, Q, |
| | | R, T, V, W, Y | R, S, T, V, W, Y |
| 56 | G | A, D, E, F, H, I, K, M, N, | A, D, E, F, G, H, I, K, M, |
| | | Q, P, R, S, T, V, W, Y | N, Q, P, R, S, T, V, W, Y |
| 57 | S | A, D, E, F, G, H, I, K, L, | A, D, E, F, G, H, I, K, L, |
| | | M, P, Q, R, T, V, Y | M, P, Q, R, S, T, V, Y |
| 58 | T | | T |
| 59 | S | A, E, F, G, H, P, Q, R, T, | A, E, F, G, H, P, Q, R, S, |
| | | Y | T, Y |
| 99 | G | A | A, G |
| 100 | G | A, H, R, S, W, Y | A, G, H, R, S, W, Y |
| 101 | W | A, E, G, H, I, K, L, M, R, | A, E, G, H, I, K, L, M, R, |
| | | V | V, W |
| 102 | I | P, R, V | I, P, R, V |
| 103 | A | E, F, H, M, P, W, Y | A, E, F, H, M, P, W, Y |
| 104 | A | | A |
| 105 | M | F, H, L, N, Y | F, H, L, M, N, Y |

### EXAMPLE 11

Exemplary HapImmune™ Antibodies Targeting Sotorasib-Peptide: MHCs Developed Based on the Cryo-EM Structures of RA_D11 in Complex with the Sotorasib-Peptide: MHC Complexes

This example provides additional antigen binding domain sequences that have improved binding affinities to the sotorasib-peptide: MHC complexes discussed above. These additional antigen binding domain sequences were developed using the deep mutational scanning discussed above.

TABLE P
Additional antigen binding domain sequences

| Name | VL | VH | LCDR1 | LCDR2 | LCDR3 | HCDR1 | HCDR2 | HCDR3 |
|---|---|---|---|---|---|---|---|---|
| RA-101 | DIQMTQSPS | EVQLVES | RASQS | SASSL | QQAS | FHWYS | SISSSW | GSWIH |
| | SLSASVGDR | GGGLVQP | VSSA | YS | YVRK | IH | GVTSY | AMDY |
| | VTITCRASQ | GGSLRLS | VA | | TIT | | ADSVK | |
| | SVSSAVAW | CAASGFT | | | | | G | |
| | YQQKPGKA | FHWYSIH | | | | | | |
| | PKLLIYSAS | WVRQAP | | | | | | |
| | SLYSGVPSR | GKGLEW | | | | | | |
| | FSGSRSGTD | VASISSS | | | | | | |
| | FTLTISSLQP | WGVTSY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGSWIH | | | | | | |
| | | AMDYWG | | | | | | |
| | | QGTLVTV | | | | | | |
| | | SS | | | | | | |
| RA-102 | DIQMTQSPS | EVQLVES | RASQS | SASSL | QQAS | FSWYSI | SIASSS | GHWIA |
| | SLSASVGDR | GGGLVQP | VSSA | YSSASS | YVRK | H | GSTGY | AMDY |
| | VTITCRASQ | GGSLRLS | VA | LY | TIT | | ADSVK | |
| | SVSSAVAW | CAASGFT | | | | | G | |
| | YQQKPGKA | FSWYSIH | | | | | | |
| | PKLLIYSAS | WVRQAP | | | | | | |
| | SLYSGVPSR | GKGLEW | | | | | | |
| | FSGSRSGTD | VASIASSS | | | | | | |
| | FTLTISSLQP | GSTGYAD | | | | | | |
| | EDFATYYC | SVKGRFT | | | | | | |
| | QQASYVRK | ISADTSKN | | | | | | |
| | TITFGQGTK | TAYLQMN | | | | | | |
| | VEIKRTV | SLRAEDT | | | | | | |
| | | AVYYCAR | | | | | | |
| | | GHWIAA | | | | | | |
| | | MDYWGQ | | | | | | |
| | | GTLVTVS | | | | | | |
| | | S | | | | | | |
| RA-103 | DIQMTQSPS | EVQLVES | RASQS | SASSL | QQAS | FSPYSI | SILSRW | GGVIH |
| | SLSASVGDR | GGGLVQP | VSSA | YS | YVRK | H | GVTSY | AMDY |
| | VTITCRASQ | GGSLRLS | VA | | TIT | | ADSVK | |
| | SVSSAVAW | CAASGFT | | | | | G | |
| | YQQKPGKA | FSPYSIH | | | | | | |
| | PKLLIYSAS | WVRQAP | | | | | | |
| | SLYSGVPSR | GKGLEW | | | | | | |
| | FSGSRSGTD | VASILSR | | | | | | |
| | FTLTISSLQP | WGVTSY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGGVIH | | | | | | |
| | | AMDYWG | | | | | | |
| | | QGTLVTV | | | | | | |
| | | SS | | | | | | |
| RA-104 | DIQMTQSPS | EVQLVES | RASQS | SASSL | QQAS | FSPYSI | SISSWH | GSWIA |
| | SLSASVGDR | GGGLVQP | VSSA | YS | YVRK | H | GETGY | AMDY |
| | VTITCRASQ | GGSLRLS | VA | | TIT | | ADSVK | |
| | SVSSAVAW | CAASGFT | | | | | G | |
| | YQQKPGKA | FSPYSIH | | | | | | |
| | PKLLIYSAS | WVRQAP | | | | | | |
| | SLYSGVPSR | GKGLEW | | | | | | |
| | FSGSRSGTD | VASISSW | | | | | | |
| | FTLTISSLQP | HGETGY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGSWIA | | | | | | |
| | | AMDYWG | | | | | | |
| | | QGTLVTV | | | | | | |
| | | SS | | | | | | |
| RA-105 | DIQMTQSPS | EVQLVES | RASQS | SASSL | QQAS | FSWYSI | SISSLQ | GGWIA |
| | SLSASVGDR | GGGLVQP | VSSA | YS | YVRK | H | GDTGY | AMDY |
| | VTITCRASQ | GGSLRLS | VA | | TIT | | ADSVK | |
| | SVSSAVAW | CAASGFT | | | | | G | |
| | YQQKPGKA | FSWYSIH | | | | | | |
| | PKLLIYSAS | WVRQAP | | | | | | |
| | SLYSGVPSR | GKGLEW | | | | | | |
| | FSGSRSGTD | VASISSLQ | | | | | | |
| | FTLTISSLQP | GDTGYA | | | | | | |
| | EDFATYYC | DSVKGRF | | | | | | |
| | QQASYVRK | TISADTSK | | | | | | |
| | TITFGQGTK | NTAYLQM | | | | | | |
| | VEIKRTV | NSLRAED | | | | | | |
| | | TAVYYCA | | | | | | |
| | | RGGWIA | | | | | | |
| | | AMDYWG | | | | | | |
| | | QGTLVTV | | | | | | |
| | | SS | | | | | | |
| RA-106 | DIQMTQSPS | EVQLVES | RASQS | SASSL | QQAS | FHYYSI | SIASW | GSWIA |
| | SLSASVGDR | GGGLVQP | VSSA | YS | YVRK | H | YGDTG | AMDY |
| | VTITCRASQ | GGSLRLS | VA | | TIT | | YADSV | |
| | SVSSAVAW | CAASGFT | | | | | KG | |
| | YQQKPGKA | FHYYSIH | | | | | | |
| | PKLLIYSAS | WVRQAP | | | | | | |
| | SLYSGVPSR | GKGLEW | | | | | | |
| | FSGSRSGTD | VASIASW | | | | | | |
| | FTLTISSLQP | YGDTGY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGSWIA | | | | | | |
| | | AMDYWG | | | | | | |
| | | QGTLVTV | | | | | | |
| | | SS | | | | | | |
| RA-107 | DIQMTQSPS | EVQLVES | RASQS | SASSL | QQAS | FGYYSI | SISSWY | GGRIE |
| | SLSASVGDR | GGGLVQP | VSSA | YS | YVRK | H | GKTGY | AMDY |
| | VTITCRASQ | GGSLRLS | VA | | TIT | | ADSVK | |
| | SVSSAVAW | CAASGFT | | | | | G | |
| | YQQKPGKA | FGYYSIH | | | | | | |
| | PKLLIYSAS | WVRQAP | | | | | | |
| | SLYSGVPSR | GKGLEW | | | | | | |
| | FSGSRSGTD | VASISSW | | | | | | |
| | FTLTISSLQP | YGKTGY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGGRIE | | | | | | |
| | | AMDYWG | | | | | | |
| | | QGTLVTV | | | | | | |
| | | SS | | | | | | |
| RA-108 | DIQMTQSPS | EVQLVES | RASQS | SASSL | QQAS | FSKYSI | SIASSY | GYWIE |
| | SLSASVGDR | GGGLVQP | VSSA | YS | YVRK | H | GSTGY | AMDY |
| | VTITCRASQ | GGSLRLS | VA | | TIT | | ADSVK | |
| | SVSSAVAW | CAASGFT | | | | | G | |
| | YQQKPGKA | FSKYSIH | | | | | | |
| | PKLLIYSAS | WVRQAP | | | | | | |
| | SLYSGVPSR | GKGLEW | | | | | | |
| | FSGSRSGTD | VASIASSY | | | | | | |
| | FTLTISSLQP | GSTGYAD | | | | | | |
| | EDFATYYC | SVKGRFT | | | | | | |
| | QQASYVRK | ISADTSKN | | | | | | |
| | TITFGQGTK | TAYLQMN | | | | | | |
| | VEIKRTV | SLRAEDT | | | | | | |
| | | AVYYCAR | | | | | | |
| | | GYWIEA | | | | | | |
| | | MDYWGQ | | | | | | |
| | | GTLVTVS | | | | | | |
| | | S | | | | | | |
| RA-109 | DIQMTQSPS | EVQLVES | RASQS | SASSL | QQAS | FGLYSI | SIHSSI | GSWIA |
| | SLSASVGDR | GGGLVQP | VSSA | YS | YVRK | H | GTTGY | AMDY |
| | VTITCRASQ | GGSLRLS | VA | | TIT | | ADSVK | |
| | SVSSAVAW | CAASGFT | | | | | G | |
| | YQQKPGKA | FGLYSIH | | | | | | |
| | PKLLIYSAS | WVRQAP | | | | | | |
| | SLYSGVPSR | GKGLEW | | | | | | |
| | FSGSRSGTD | VASIHSSI | | | | | | |
| | FTLTISSLQP | GTTGYA | | | | | | |
| | EDFATYYC | DSVKGRF | | | | | | |
| | QQASYVRK | TISADTSK | | | | | | |
| | TITFGQGTK | NTAYLQM | | | | | | |
| | VEIKRTV | NSLRAED | | | | | | |
| | | TAVYYCA | | | | | | |
| | | RGSWIAA | | | | | | |
| | | MDYWGQ | | | | | | |
| | | GTLVTVS | | | | | | |
| | | S | | | | | | |
| RA-110 | DIQMTQSPS | EVQLVES | RASQS | SASSL | QQAS | FSPYSI | SILSWI | GSVIH |
| | SLSASVGDR | GGGLVQP | VSSA | YS | YVRK | H | GKTSY | AMDY |
| | VTITCRASQ | GGSLRLS | VA | | TIT | | ADSVK | |
| | SVSSAVAW | CAASGFT | | | | | G | |
| | YQQKPGKA | FSPYSIH | | | | | | |
| | PKLLIYSAS | WVRQAP | | | | | | |
| | SLYSGVPSR | GKGLEW | | | | | | |
| | FSGSRSGTD | VASILSW | | | | | | |
| | FTLTISSLQP | IGKTSYA | | | | | | |
| | EDFATYYC | DSVKGRF | | | | | | |
| | QQASYVRK | TISADTSK | | | | | | |
| | TITFGQGTK | NTAYLQM | | | | | | |
| | VEIKRTV | NSLRAED | | | | | | |
| | | TAVYYCA | | | | | | |
| | | RGSVIHA | | | | | | |
| | | MDYWGQ | | | | | | |
| | | GTLVTVS | | | | | | |
| | | S | | | | | | |
| RA-111 | DIQMTQSPS | EVQLVES | RASQS | SASSL | QQAS | FSPYHI | SIASR | GGWIA |
| | SLSASVGDR | GGGLVQP | VSSA | YS | YVRK | H | WGHT | AMDY |
| | VTITCRASQ | GGSLRLS | VA | | TIT | | GYADS | |
| | SVSSAVAW | CAASGFT | | | | | VKG | |
| | YQQKPGKA | FSPYHIH | | | | | | |
| | PKLLIYSAS | WVRQAP | | | | | | |
| | SLYSGVPSR | GKGLEW | | | | | | |
| | FSGSRSGTD | VASIASR | | | | | | |
| | FTLTISSLQP | WGHTGY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGGWI | | | | | | |
| | | AAMDYW | | | | | | |
| | | GQGTLVT | | | | | | |
| | | VSS | | | | | | |
| RA-112 | DIQMTQSPS | EVQLVES | RASQS | SASSL | QQAS | FHEYSI | SIASLQ | GSWIA |
| | SLSASVGDR | GGGLVQP | VSSA | YS | YVRK | H | GITGY | AMDY |
| | VTITCRASQ | GGSLRLS | VA | | TIT | | ADSVK | |
| | SVSSAVAW | CAASGFT | | | | | G | |
| | YQQKPGKA | FHEYSIH | | | | | | |
| | PKLLIYSAS | WVRQAP | | | | | | |
| | SLYSGVPSR | GKGLEW | | | | | | |
| | FSGSRSGTD | VASIASL | | | | | | |
| | FTLTISSLQP | QGITGYA | | | | | | |
| | EDFATYYC | DSVKGRF | | | | | | |
| | QQASYVRK | TISADTSK | | | | | | |
| | TITFGQGTK | NTAYLQM | | | | | | |
| | VEIKRTV | NSLRAED | | | | | | |
| | | TAVYYCA | | | | | | |
| | | RGSWIAA | | | | | | |
| | | MDYWGQ | | | | | | |
| | | GTLVTVS | | | | | | |
| | | S | | | | | | |
| RA-203 | DIQMTQSPS | EVQLVES | RASQS | SASSL | QQAS | FSDYSI | SILSRW | GGVIH |
| | SLSASVGDR | GGGLVQP | VSSA | YS | YVRK | H | GVTSY | AMDY |
| | VTITCRASQ | GGSLRLS | VA | | TIT | | ADSVK | |
| | SVSSAVAW | CAASGFT | | | | | G | |
| | YQQKPGKA | FSDYSIH | | | | | | |
| | PKLLIYSAS | WVRQAP | | | | | | |
| | SLYSGVPSR | GKGLEW | | | | | | |
| | FSGSRSGTD | VASILSR | | | | | | |
| | FTLTISSLQP | WGVTSY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGGVIH | | | | | | |
| | | AMDYWG | | | | | | |
| | | QGTLVTV | | | | | | |
| | | SS | | | | | | |
| RA-303 | DIQMTQSPS | EVQLVES | RASQS | SASSL | QQAS | FSDYSI | SISSRW | GGVIH |
| | SLSASVGDR | GGGLVQP | VSSA | YS | YVRK | H | GVTSY | AMDY |
| | VTITCRASQ | GGSLRLS | VA | | TIT | | ADSVK | |
| | SVSSAVAW | CAASGFT | | | | | G | |
| | YQQKPGKA | FSDYSIH | | | | | | |
| | PKLLIYSAS | WVRQAP | | | | | | |
| | SLYSGVPSR | GKGLEW | | | | | | |
| | FSGSRSGTD | VASISSR | | | | | | |
| | FTLTISSLQP | WGVTSY | | | | | | |
| | EDFATYYC | ADSVKGR | | | | | | |
| | QQASYVRK | FTISADTS | | | | | | |
| | TITFGQGTK | KNTAYLQ | | | | | | |
| | VEIKRTV | MNSLRAE | | | | | | |
| | | DTAVYYC | | | | | | |
| | | ARGGVIH | | | | | | |
| | | AMDYWG | | | | | | |
| | | QGTLVTV | | | | | | |
| | | SS | | | | | | |

TABLE Q
Summary of binding partner sequences described herein.

| Name | VL | VH | LCDR1 | LCDR2 | LCDR3 | HCDR1 | HCDR2 | HCDR3 |
|---|---|---|---|---|---|---|---|---|
| OEA2-5 | DIQMTQSPS | EVQLVESG | RASQS | SASSL | QQYS | SSYIH | YISPSY | EYVTM |
| | SLSASVGDR | GGLVQPG | VSSAV | YS | YWPIT | | GSTSY | ALDY |
| | VTITCRASQ | GSLRLSCA | A | | | | ADSVK | |
| | SVSSAVAW | ASGFTISSS | | | | | G | |
| | YQQKPGKA | YIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSYWPI | AYLQMNS | | | | | | |
| | TFGQGTKV | LRAEDTAV | | | | | | |
| | EIKRTV | YYCAREY | | | | | | |
| | | VTMALDY | | | | | | |
| | | WGQGTLV | | | | | | |
| | | TVSS | | | | | | |
| EO_Q01 | DIQMTQSPS | EVQLVESG | RASQS | SASSL | QQYS | SSYIH | YISPSY | ELVTM |
| | SLSASVGDR | GGLVQPG | VSSAV | YS | HWPIT | | GSTSY | TADY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCAREL | | | | | | |
| | | VTMTADY | | | | | | |
| | | WGQGTLV | | | | | | |
| | | TVSS | | | | | | |
| EO_Q02 | DIQMTQSPS | EVQLVESG | RASQS | SASSL | QQYS | SSYIH | YISPSY | EYTTM |
| | SLSASVGDR | GGLVQPG | VSSAV | YS | AWPE | | GSTSY | SIDY |
| | VTITCRASQ | GSLRLSCA | A | | T | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSAWPE | AYLQMNS | | | | | | |
| | TFGQGTKV | LRAEDTAV | | | | | | |
| | EIKRTV | YYCAREY | | | | | | |
| | | TTMSIDYW | | | | | | |
| | | GQGTLVT | | | | | | |
| | | VSS | | | | | | |
| EO_Q03 | DIQMTQSPS | EVQLVESG | RASQS | SASSL | QQYS | SSYIH | YISPSY | ESVTM |
| | SLSASVGDR | GGLVQPG | VSSAV | YS | AWPIT | | GSTSY | SADY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSAWPI | AYLQMNS | | | | | | |
| | TFGQGTKV | LRAEDTAV | | | | | | |
| | EIKRTV | YYCARESV | | | | | | |
| | | TMSADYWG | | | | | | |
| | | QGTLVT | | | | | | |
| | | VSS | | | | | | |
| EO_Q04 | DIQMTQSPS | EVQLVESG | RASQS | SASSL | QQYS | SSYIH | YISPSY | ESVTM |
| | SLSASVGDR | GGLVQPG | VSSAV | YS | DWPIT | | GSTSY | TKDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSDWPI | AYLQMNS | | | | | | |
| | TFGQGTKV | LRAEDTAV | | | | | | |
| | EIKRTV | YYCARESV | | | | | | |
| | | TMTKDYWG | | | | | | |
| | | QGTLVTVSS | | | | | | |
| EO_Q05 | DIQMTQSPS | EVQLVESG | RASQS | SASSL | QQYA | SSYIH | YISPSY | EEVTM |
| | SLSASVGDR | GGLVQPG | VSSAV | YS | EWPIT | | GSTSY | SIDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYAEWPI | AYLQMNS | | | | | | |
| | TFGQGTKV | LRAEDTAV | | | | | | |
| | EIKRTV | YYCAREE | | | | | | |
| | | VTMSIDY | | | | | | |
| | | WGQGTLV | | | | | | |
| | | TVSS | | | | | | |
| EO_Q06 | DIQMTQSPS | EVQLVESG | RASQS | SASSL | QQYS | SSYIH | YISPSY | ESITMT |
| | SLSASVGDR | GGLVQPG | VSSAV | YS | HWPIT | | GSTSY | KDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCARESI | | | | | | |
| | | TMTKDYW | | | | | | |
| | | GQGTLVT | | | | | | |
| | | VSS | | | | | | |
| EO_Q | DIQMTQSPS | EVQLVESG | RASQS | SASSL | QQYA | SSYIH | YISPSY | ELTTM |
| 07 | SLSASVGDR | GGLVQPG | VSSAV | YS | KWPIT | | GSTSY | SIDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYAKWPI | AYLQMNS | | | | | | |
| | TFGQGTKV | LRAEDTAV | | | | | | |
| | EIKRTV | YYCARELT | | | | | | |
| | | TMSIDYW | | | | | | |
| | | GQGTLVT | | | | | | |
| | | VSS | | | | | | |
| EO_Q | DIQMTQSPS | EVQLVESG | RASQS | SASSL | QQYS | SSYIH | YISPSY | ELTEM |
| 08 | SLSASVGDR | GGLVQPG | VSSAV | YS | SWPIT | | GSTSY | TTDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSSWPIT | AYLQMNS | | | | | | |
| | FGQGTKVEI | LRAEDTAV | | | | | | |
| | KRTV | YYCARELT | | | | | | |
| | | EMTTDYW | | | | | | |
| | | GQGTLVT | | | | | | |
| | | VSS | | | | | | |
| EO_Q | DIQMTQSPS | EVQLVESG | RASQS | SASSL | QQYS | SSYIH | YISPSY | EEVTM |
| 09 | SLSASVGDR | GGLVQPG | VSSAV | YS | HWPIT | | GSTSY | TADY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCAREE | | | | | | |
| | | VTMTADY | | | | | | |
| | | WGQGTLV | | | | | | |
| | | TVSS | | | | | | |
| EO_Q10 | DIQMTQSPS | EVQLVESG | RASQS | SASSL | QQYS | SSYIH | YISPSY | ELIEM |
| | SLSASVGDR | GGLVQPG | VSSAV | YS | GWPIT | | GSTSY | TPDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSGWPI | AYLQMNS | | | | | | |
| | TFGQGTKV | LRAEDTAV | | | | | | |
| | EIKRTV | YYCARELI | | | | | | |
| | | EMTPDYW | | | | | | |
| | | GQGTLVT | | | | | | |
| | | VSS | | | | | | |
| EO_Q11 | DIQMTQSPS | EVQLVESG | RASQS | SASSL | QQYS | SSYIH | YISPSY | ELVTM |
| | SLSASVGDR | GGLVQPG | VSSAV | YS | GWPIT | | GSTSY | TIDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSGWPI | AYLQMNS | | | | | | |
| | TFGQGTK | LRAEDTAV | | | | | | |
| | VEIKRTV | YYCAREL | | | | | | |
| | | VTMTIDY | | | | | | |
| | | WGQGTLV | | | | | | |
| | | TVSS | | | | | | |
| EO_Q12 | DIQMTQSPS | EVQLVESG | RASQS | SASSLYS | QQYA | SSYIH | YISPSY | ELTTMSIDY |
| | SLSASVGDR | GGLVQPGG | VSSAV | | SWPIT | | GSTSYA | |
| | VTITCRASQ | SLRLSCA | A | | | | DSVKG | |
| | SVSSAVAW | ASGFTISS | | | | | | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYASWPI | AYLQMNSL | | | | | | |
| | TFGQGTKV | RAEDTAVY | | | | | | |
| | EIKRTV | YCARELTT | | | | | | |
| | | MSIDYWGQ | | | | | | |
| | | GTLVTVSS | | | | | | |
| EO_Q13 | DIQMTQSPS | EVQLVESG | RASQS | SASSL | QQYS | SSYIH | YISPSYGS | ELTTMTADY |
| | SLSASVGDR | GGLVQPG | VSSAV | YS | HWPIT | | TSYADSVKG | |
| | VTITCRASQ | GSLRLSCA | A | | | | | |
| | SVSSAVAWY | ASGFTISS | | | | | | |
| | QQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCARELT | | | | | | |
| | | TMTADYWG | | | | | | |
| | | QGTLVTVSS | | | | | | |
| EO_Q | DIQMTQSPS | EVQLVESG | RASQS | SASSL | QQYS | SSYIH | YISPSY | ESITMS |
| 14 | SLSASVGDR | GGLVQPG | VSSAV | YS | SWPIT | | GSTSY | PDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSSWPIT | AYLQMNS | | | | | | |
| | FGQGTKVEI | LRAEDTAV | | | | | | |
| | KRTV | YYCARESI | | | | | | |
| | | TMSPDYW | | | | | | |
| | | GQGTLVT | | | | | | |
| | | VSS | | | | | | |
| EO_Q | DIQMTQSPS | EVQLVESG | RASQS | SASSL | QQYS | SSYIH | YISPSY | ELIEM |
| 15 | SLSASVGDR | GGLVQPG | VSSAV | YS | HWPIT | | GSTSY | TTDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCARELI | | | | | | |
| | | EMTTDYW | | | | | | |
| | | GQGTLVT | | | | | | |
| | | VSS | | | | | | |
| EO_Q | DIQMTQSPS | EVQLVESG | RASQS | SASSL | QQYS | SSYIH | YISPSY | EYVTM |
| 16 | SLSASVGDR | GGLVQPG | VSSAV | YS | DWPIT | | GSTSY | SIDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSDWPI | AYLQMNS | | | | | | |
| | TFGQGTKV | LRAEDTAV | | | | | | |
| | EIKRTV | YYCAREYV | | | | | | |
| | | TMSIDYWG | | | | | | |
| | | QGTLVTVSS | | | | | | |
| EO_Q | DIQMTQSPS | EVQLVESG | RASQS | SASSL | QQYS | SSYIH | YISPSY | ELVEM |
| 17 | SLSASVGDR | GGLVQPG | VSSAV | YS | SWPIT | | GSTSY | TPDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSSWPIT | AYLQMNS | | | | | | |
| | FGQGTKVEI | LRAEDTAV | | | | | | |
| | KRTV | YYCAREL | | | | | | |
| | | VEMTPDYW | | | | | | |
| | | GQGTLVTV | | | | | | |
| | | SS | | | | | | |
| EO_Q | DIQMTQSPS | EVQLVESG | RASQS | SASSL | QQYS | SSYIH | YISPSY | EYVTM |
| 18 | SLSASVGDR | GGLVQPG | VSSAV | YS | AWPE | | GSTSY | ALDY |
| | VTITCRASQ | GSLRLSCA | A | | T | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSAWPE | AYLQMNS | | | | | | |
| | | LRAEDTAV | | | | | | |
| | TFGQGT | YYCARE | | | | | | |
| | KVEIKR | YVTMAL | | | | | | |
| | TV | DYWGQG | | | | | | |
| | | TLVTVSS | | | | | | |
| EO_Q20 | DIQMTQSPS | EVQLVESG | RASQS | SASSL | QQYS | SSYIH | YISPSY | EEVTM |
| | SLSASVGDR | GGLVQPG | VSSAV | YS | DWPIT | | GSTSY | TPDY |
| | VTITCRASQ | GSLRLSCA | A | | | | ADSVK | |
| | SVSSAVAW | ASGFTISS | | | | | G | |
| | YQQKPGKA | SYIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSDWPI | AYLQMNS | | | | | | |
| | TFGQGTKV | LRAEDTAV | | | | | | |
| | EIKRTV | YYCAREE | | | | | | |
| | | VTMTPDY | | | | | | |
| | | WGQGTLV | | | | | | |
| | | TVSS | | | | | | |
| EO_Q22 | DIQMTQSPS | EVQLVESG | RASQS | SASSL | QQYS | SSYIH | YISPSY | EYVTM |
| | SLSASVGDR | GGLVQPG | VSSAV | YS | HWPIT | | GSTSY | ALDY |
| | VTITCRASQ | GSLRLSCA | A | | | | ADSVK | |
| | SVSSAVAW | ASGFTISSS | | | | | G | |
| | YQQKPGKA | YIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPI | AYLQMNS | | | | | | |
| | TFGQGTKV | LRAEDTAV | | | | | | |
| | EIKRTV | YYCAREY | | | | | | |
| | | VTMALDY | | | | | | |
| | | WGQGTLV | | | | | | |
| | | TVSS | | | | | | |
| EO_Q | DIQMTQSPS | EVQLVESG | RASQS | SASSL | QQYS | SSYIH | YISPSY | EYVTM |
| 23 | SLSASVGDR | GGLVQPG | VSSAV | YS | HWPE | | GSTSY | ALDY |
| | VTITCRASQ | GSLRLSCA | A | | T | | ADSVK | |
| | SVSSAVAW | ASGFTISSS | | | | | G | |
| | YQQKPGKA | YIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSHWPE | AYLQMNS | | | | | | |
| | TFGQGTKV | LRAEDTAV | | | | | | |
| | EIKRTV | YYCAREY | | | | | | |
| | | VTMALDY | | | | | | |
| | | WGQGTLV | | | | | | |
| | | TVSS | | | | | | |
| EO_Q | DIQMTQSPS | EVQLVESG | RASQS | SASSL | QQYS | SSYIH | YISPSY | EYVTM |
| 24 | SLSASVGDR | GGLVQPG | VSSAV | YS | EWPE | | GSTSY | ALDY |
| | VTITCRASQ | GSLRLSCA | A | | T | | ADSVK | |
| | SVSSAVAW | ASGFTISSS | | | | | G | |
| | YQQKPGKA | YIHWVRQ | | | | | | |
| | PKLLIYSAS | APGKGLE | | | | | | |
| | SLYSGVPSR | WVAYISPS | | | | | | |
| | FSGSRSGTD | YGSTSYAD | | | | | | |
| | FTLTISSLQ | SVKGRFTI | | | | | | |
| | PEDFATYYC | SADTSKNT | | | | | | |
| | QQYSEWPE | AYLQMNS | | | | | | |
| | TFGQGTKV | LRAEDTAV | | | | | | |
| | EIKRTV | YYCAREY | | | | | | |
| | | VTMALDY | | | | | | |
| | | WGQGTLV | | | | | | |
| | | TVSS | | | | | | |
| AMR | DIQMTQSPS | EVQLVESG | RASQS | SASSL | QQISY | DYSIH | SISSSS | GGWY |
| A3-7D | SLSASVGDR | GGLVQPG | VSSAV | YS | VKKLI | | GSTSY | PAMDY |
| | VTITCRASQ | GSLRLSCA | A | | T | | ADSVK | |
| | SVSSAVA | ASGFTFS | | | | | G | |
| | WYQQKPG | DYSIHWV | | | | | | |
| | KAPKLL | RQAPGKG | | | | | | |
| | IYSASSL | LEWVASI | | | | | | |
| | YSGVPSRF | SSSSGSTS | | | | | | |
| | SGSRSGTD | YADSVKG | | | | | | |
| | FTLTISSL | RFTISAD | | | | | | |
| | QPEDFATY | TSKNTAY | | | | | | |
| | YCQQISYVK | LQMNSLR | | | | | | |
| | KLITFGQGT | AEDTAVY | | | | | | |
| | VEIKRTV | YCARGGWY | | | | | | |
| | | PAMDYWGQ | | | | | | |
| | | GTLVTVSS | | | | | | |
| RAD01 | DIQMTQSPS | EVQLVESG | RASQS | SASSL | QQAS | DYSIH | SISSSS | GGWIA |
| | SLSASVGDR | GGLVQPG | VSSAV | YS | YVRR | | GSTSY | AMDY |
| | VTITCRASQ | GSLRLSCA | A | | TIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | TITFGQGTK | LRAEDTAV | | | | | | |
| | VEIKRTV | YYCARGG | | | | | | |
| | | WIAAMDYW | | | | | | |
| | | GQGTLVTV | | | | | | |
| | | SS | | | | | | |
| RAD | DIQMTQSPS | EVQLVESG | RASQS | SASSL | QQPS | DYSIH | SISSSS | GRWIP |
| 02 | SLSASVGDR | GGLVQPG | VSSAV | YS | YVRR | | GSTSY | AMDY |
| | VTITCRASQ | GSLRLSCA | A | | KIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQPSYVRR | AYLQMNS | | | | | | |
| | KITFGQGTK | LRAEDTAV | | | | | | |
| | VEIKRTV | YYCARGR | | | | | | |
| | | WIPAMDYW | | | | | | |
| | | GQGTLVTV | | | | | | |
| | | SS | | | | | | |
| RAD03 | DIQMTQSPS | EVQLVESG | RASQS | SASSL | QQAS | DYSIH | SISSSS | GGWIA |
| | SLSASVGDR | GGLVQPG | VSSAV | YS | YVAR | | GSTSY | AMDY |
| | VTITCRASQ | GSLRLSCA | A | | KIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVAR | AYLQMNS | | | | | | |
| | KITFGQGTK | LRAEDTAV | | | | | | |
| | VEIKRTV | YYCARGG | | | | | | |
| | | WIAAMDY | | | | | | |
| | | WGQGTLV | | | | | | |
| | | TVSS | | | | | | |
| RAD04 | DIQMTQSPS | EVQLVESG | RASQS | SASSL | QQVS | DYSIH | SISSSS | GRWIP |
| | SLSASVGDR | GGLVQPG | VSSAV | YS | YVAR | | GSTSY | AMDY |
| | VTITCRASQ | GSLRLSCA | A | | RIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQVSYVAR | AYLQMNS | | | | | | |
| | RITFGQGTK | LRAEDTAV | | | | | | |
| | VEIKRTV | YYCARGR | | | | | | |
| | | WIPAMDY | | | | | | |
| | | WGQGTLV | | | | | | |
| | | TVSS | | | | | | |
| RAD | DIQMTQSPS | EVQLVESG | RASQS | SASSL | QQAS | DYSIH | SISSSS | GHWIA |
| 06 | SLSASVGDR | GGLVQPG | VSSAV | YS | YVKR | | GSTSY | AMDY |
| | VTITCRASQ | GSLRLSCA | A | | LIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVKR | AYLQMNS | | | | | | |
| | LITFGQGTK | LRAEDTAV | | | | | | |
| | VEIKRTV | YYCARGH | | | | | | |
| | | WIAAMDYW | | | | | | |
| | | GQGTLVTV | | | | | | |
| | | SS | | | | | | |
| RAD | DIQMTQSPS | EVQLVESG | RASQS | SASSL | QQAS | DYSIH | SISSSS | GRWIP |
| 07 | SLSASVGDR | GGLVQPG | VSSAV | YS | YVRR | | GSTSY | AMDY |
| | VTITCRASQ | GSLRLSCA | A | | LIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | LITFGQGTK | LRAEDTAV | | | | | | |
| | VEIKRTV | YYCARGR | | | | | | |
| | | WIPAMDY | | | | | | |
| | | WGQGTLV | | | | | | |
| | | TVSS | | | | | | |
| RAD | DIQMTQSPS | EVQLVESG | RASQS | SASSL | QQAS | DYSIH | SISSSS | GGWIA |
| 08 | SLSASVGDR | GGLVQPG | VSSAV | YS | YVRR | | GSTSY | AMDY |
| | VTITCRASQ | GSLRLSCA | A | | VIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | EPDFATYYC | SADTSKNT | | | | | | |
| | QQASYVRR | AYLQMNS | | | | | | |
| | VITFGQGTK | LRAEDTAV | | | | | | |
| | VEIKRTV | YYCARGGWI | | | | | | |
| | | AAMDYWGQ | | | | | | |
| | | GTLVTVSS | | | | | | |
| RAD09 | DIQMTQSPS | EVQLVESG | RASQS | SASSL | QQAS | DYSIH | SISSSS | GSWIA |
| | SLSASVGDR | GGLVQPG | VSSAV | YS | YVRR | | GSTSY | AMDY |
| | VTITCRASQ | GSLRLSCA | A | | TIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | TITFGQGTK | LRAEDTAV | | | | | | |
| | VEIKRTV | YYCARGS | | | | | | |
| | | WIAAMDY | | | | | | |
| | | WGQGTLV | | | | | | |
| | | TVSS | | | | | | |
| RAD11 | DIQMTQSPS | EVQLVESG | RASQS | SASSL | QQAS | DYSIH | SISSSS | GGWIA |
| | SLSASVGDR | GGLVQPG | VSSAV | YS | YVRK | | GSTSY | AMDY |
| | VTITCRASQ | GSLRLSCA | A | | TIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRK | AYLQMNS | | | | | | |
| | | LRAEDTAV | | | | | | |
| | TITFGQGTK | YYCARG | | | | | | |
| | VEIKRTV | GWIAAM | | | | | | |
| | | DYWGQG | | | | | | |
| | | TLVTVSS | | | | | | |
| RAD12 | DIQMTQSPS | EVQLVESG | RASQS | SASSL | QQAS | DYSIH | SISSSS | GRWIP |
| | SLSASVGDR | GGLVQPG | VSSAV | YS | YVAK | | GSTSY | AMDY |
| | VTITCRASQ | GSLRLSCA | A | | | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | TLTISSLQP | SVKGRFTI | | | | | | |
| | EDFATYYCQ | SADTSKNT | | | | | | |
| | QASYVAKTI | AYLQMNS | | | | | | |
| | TFGQGTKV | LRAEDTAV | | | | | | |
| | EIKRTV | YYCARGR | | | | | | |
| | | WIPAMDYW | | | | | | |
| | | GQGTLVTV | | | | | | |
| | | SS | | | | | | |
| RAD13 | DIQMTQSPS | EVQLVESG | RASQS | SASSL | QQSS | DYSIH | SISSSS | GKWIP |
| | SLSASVGDR | GGLVQPG | VSSAV | YS | YVRR | | GSTSY | AMDY |
| | VTITCRAGQ | GSLRLSCA | A | | KIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSYVRR | AYLQMNS | | | | | | |
| | KITFGQGTK | LRAEDTAV | | | | | | |
| | VEIKRTV | YYCARGK | | | | | | |
| | | WIPAMDY | | | | | | |
| | | WGQGTLVT | | | | | | |
| | | VSS | | | | | | |
| RA_D | DIQMTQSPS | EVQLVESG | RASQS | SASSL | QQAS | DYSIH | SISSSS | GGWIA |
| 14 | SLSASVGDR | GGLVQPG | VSSAV | YS | YVRK | | GSTSY | AMDY |
| | VTITCRASQ | GSLRLSCA | A | | AIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRK | AYLQMNS | | | | | | |
| | AITFGQGTK | LRAEDTAV | | | | | | |
| | VEIKRTV | YYCARGG | | | | | | |
| | | WIAAMDYW | | | | | | |
| | | GQGTLVTV | | | | | | |
| | | SS | | | | | | |
| RAD | DIQMTQSPS | EVQLVESG | RASQS | SASSL | QQAS | DYSIH | SISSSS | GRWIP |
| 16 | SLSASVGDR | GGLVQPG | VSSAV | YS | YVRR | | GSTSY | AMDY |
| | VTITCRASQ | GSLRLSCA | A | | AIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | AITFGQGTK | LRAEDTAV | | | | | | |
| | VEIKRTV | YYCARGR | | | | | | |
| | | WIPAMDY | | | | | | |
| | | WGQGTLV | | | | | | |
| | | TVSS | | | | | | |
| RAD | DIQMTQSPS | EVQLVESG | RASQS | SASSL | QQSS | DYSIH | SISSSS | GRWIP |
| 18 | SLSASVGDR | GGLVQPG | VSSAV | YS | YVKR | | GSTSY | AMDY |
| | VTITCRASQ | GSLRLSCA | A | | TIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSYVKR | AYLQMNS | | | | | | |
| | TITFGQGTK | LRAEDTAV | | | | | | |
| | VEIKRTV | YYCARGR | | | | | | |
| | | WIPAMDY | | | | | | |
| | | WGQGTLV | | | | | | |
| | | TVSS | | | | | | |
| RAD | DIQMTQSPS | EVQLVESG | RASQS | SASSL | QQPS | DYSIH | SISSSS | GRWIP |
| 19 | SLSASVGDR | GGLVQPG | VSSAV | YS | YVRK | | GSTSY | AMDY |
| | VTITCRASQ | GSLRLSCA | A | | TIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQPSYVRK | AYLQMNS | | | | | | |
| | TITFGQGTK | LRAEDTAV | | | | | | |
| | VEIKRTV | YYCARGR | | | | | | |
| | | WIPAMDYW | | | | | | |
| | | GQGTLVTVS | | | | | | |
| | | S | | | | | | |
| RAD20 | DIQMTQSPS | EVQLVESG | RASQS | SASSL | QQAS | DYSIH | SISSSS | GRWIP |
| | SLSASVGDR | GGLVQPG | VSSAV | YS | YVKK | | GSTSY | AMDY |
| | VTITCRASQ | GSLRLSCA | A | | EIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVKK | AYLQMNS | | | | | | |
| | EITFGQG | LRAEDTAV | | | | | | |
| | TKVEIKR | YYCARGRW | | | | | | |
| | TV | IPAMDYWG | | | | | | |
| | | QGTLVTVSS | | | | | | |
| RAD21 | DIQMTQSPS | EVQLVESG | RASQS | SASSLYS | QQSS | DYSIH | SISSSS | GRWIP |
| | SLSASVGDR | GGLVQPG | VSSAV | | YVHK | | GSTSYA | AMDY |
| | VTITCRASQ | GSLRLSCA | A | | LIT | | DSVKG | |
| | SVSSAVAW | ASGFTFSD | | | | | | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSYVHK | AYLQMNS | | | | | | |
| | LITFGQGTK | LRAEDTAV | | | | | | |
| | VEIKRTV | YYCARGR | | | | | | |
| | | WIPAMDY | | | | | | |
| | | WGQGTLV | | | | | | |
| | | TVSS | | | | | | |
| RAD23 | DIQMTQSPS | EVQLVESG | RASQS | SASSL | QQAS | DYSIH | SISSSS | GHWIA |
| | SLSASVGDR | GGLVQPG | VSSAV | YS | YVRR | | GSTSY | AMDY |
| | VTITCRASQ | GSLRLSCA | A | | EIT | | ADSVKG | |
| | SVSSAVAW | ASGFTFSD | | | | | | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | EITFGQGTK | LRAEDTAV | | | | | | |
| | VEIKRTV | YYCARGH | | | | | | |
| | | WIAAMDY | | | | | | |
| | | WGQGTLV | | | | | | |
| | | TVSS | | | | | | |
| RAD | DIQMTQSPS | EVQLVESG | RASQS | SASSL | QQAS | DYSIH | SISSSS | GRWIP |
| 24 | SLSASVGDR | GGLVQPG | VSSAV | YS | YVHR | | GSTSY | AMDY |
| | VTITCRASQ | GSLRLSCA | A | | LIT | | ADSVK | |
| | SVSSAVAW | ASGFTFSD | | | | | G | |
| | YQQKPGKA | YSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVHR | AYLQMNS | | | | | | |
| | LITFGQGTK | LRAEDTAV | | | | | | |
| | VEIKRTV | YYCARGRWI | | | | | | |
| | | PAMDYWGQG | | | | | | |
| | | TLVTVSS | | | | | | |
| AMR | DIQMTQSPS | EVQLVESG | RASQS | SASSL | QQSG | SSYIH | SISPYY | YYALD |
| A3-2 | SLSASVGDR | GGLVQPG | VSSAV | YS | WSYPI | | GSTSY | Y |
| | VTITCRASQ | GSLRLSCA | A | | T | | ADSVK | |
| | SVSSAVAW | ASGFTFYS | | | | | G | |
| | YQQKPGKA | SYIHWVR | | | | | | |
| | PKLLIYSAS | QAPGKGLE | | | | | | |
| | SLYSGVPSR | WVASISPY | | | | | | |
| | FSGSRSGTD | YGSTSYA | | | | | | |
| | FTLTISSLQ | DSVKGRFT | | | | | | |
| | PEDFATYYC | ISADTSKN | | | | | | |
| | QQSGWSYP | TAYLQMN | | | | | | |
| | ITFGQGTKV | SLRAEDTA | | | | | | |
| | EIKRTV | VYYCARSS | | | | | | |
| | | YYALDYW | | | | | | |
| | | GQGTLVT | | | | | | |
| | | VSS | | | | | | |
| AMRA3-7 | DIQMTQSPS | EVQLVESG | RASQS | SASSL | QQISY | SYSIH | SIYSSY | GGWY |
| | SLSASVGDR | GGLVQPG | VSSAV | YS | VYSLI | | GYTSY | PAMDY |
| | VTITCRASQ | GSLRLSCA | A | | T | | ADSVK | |
| | SVSSAVAW | ASGFTFSS | | | | | G | |
| | YQQKPGKA | YSIHWVR | | | | | | |
| | PKLLIYSAS | QAPGKGLE | | | | | | |
| | SLYSGVPSR | WVASIYSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQISYVYS | TAYLQMN | | | | | | |
| | LITFGQGTK | SLRAEDTA | | | | | | |
| | VEIKRTV | VYYCARG | | | | | | |
| | | GWYPAMD | | | | | | |
| | | YWGQGTLV | | | | | | |
| | | TVSS | | | | | | |
| AMR | DIQMTQSPS | EVQLVESG | RASQS | SASSL | QQISY | SYSIH | SIYSSY | GGWY |
| A3- | SLSASVGDR | GGLVQPG | VSSAV | YS | VKKLI | | GYTSY | PAMDY |
| 7KK | VTITCRASQ | GSLRLSCA | A | | T | | ADSVK | |
| | SVSSAVAW | ASGFTFSS | | | | | G | |
| | YQQKPGKA | YSIHWVR | | | | | | |
| | PKLLIYSAS | QAPGKGLE | | | | | | |
| | SLYSGVPSR | WVASIYSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQISYVKK | TAYLQMN | | | | | | |
| | LITFGQGTK | SLRAEDTA | | | | | | |
| | VEIKRTV | VYYCARG | | | | | | |
| | | GWYPAM | | | | | | |
| | | DYWGQGT | | | | | | |
| | | LVTVSS | | | | | | |
| AMR | DIQMTQSPS | EVQLVESG | RASQS | SASSL | QQISY | DYSIH | SISSSS | GGWY |
| A3-7D | SLSASVGDR | GGLVQPG | VSSAV | YS | VKKLI | | GSTSY | PAMDY |
| | VTITCRASQ | GSLRLSCA | A | | T | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQISYVKK | AYLQMNS | | | | | | |
| | LITFGQ | LRAEDTA | | | | | | |
| | GTKVEIKR | VYYCARGG | | | | | | |
| | TV | WYPAMDY | | | | | | |
| | | WGQGTLVT | | | | | | |
| | | VSS | | | | | | |
| AMRA3-8 | DIQMTQSPS | EVQLVESG | RASQS | SASSL | QQYQ | YSSIH | SIYSYS | YSYG |
| | SLSASVGDR | GGLVQPG | VSSAV | YS | YGYN | | GSTSY | WVGP |
| | VTITCRASQ | GSLRLSCA | A | | LIT | | ADSVK | GWRAI |
| | SVSSAVAW | ASGFTISYS | | | | | G | DY |
| | YQQKPGKA | SIHWVRQ | | | | | | |
| | PKLLIYSAS | APGKGLE | | | | | | |
| | SLYSGVPSR | WVASIYSY | | | | | | |
| | FSGSRSGTD | SGSTSYAD | | | | | | |
| | FTLTISSLQ | SVKGRFTI | | | | | | |
| | PEDLATYYC | SADTSKNT | | | | | | |
| | QQYQYGY | AYLQMNS | | | | | | |
| | NLITFGQGT | LRAEDTAV | | | | | | |
| | KVEIKRTV | YYCARYS | | | | | | |
| | | YGWVGPGW | | | | | | |
| | | RAIDYWGQ | | | | | | |
| | | GTLVTVSS | | | | | | |
| AMRA | DIQMTQSPS | EVQLVESG | RASQS | SASSL | QQSSS | YSSIH | SISSSY | GGPG |
| 3-11 | SLSASVGDR | GGLVQPG | VSSAV | YS | VYKL | | SYTSY | WYRA |
| | VTITCRASQ | GSLRLSCA | A | | LT | | ADSVK | MDY |
| | SVSSAVAW | ASGFTVYY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YSYTSYAD | | | | | | |
| | FTLTISSLQ | SVKGRFTI | | | | | | |
| | PEDFATYYC | SADTSKNT | | | | | | |
| | QQSSSVYK | AYLQMNS | | | | | | |
| | LLTFGQGT | LRAEDTAL | | | | | | |
| | KVEIKRTV | YYCARGG | | | | | | |
| | | PGWYRA | | | | | | |
| | | MDYWGQ | | | | | | |
| | | GTLVTVSS | | | | | | |
| AMRA | DIQMTQSPS | EVQLVESG | RASQS | SASSL | QQSSS | SSSIH | SISSSS | GYFYY |
| 3-15 | SLSASVGDR | GGLVQPG | VSSAV | YS | SLIT | | GSTSY | GWWA |
| | VTITCRASQ | GSLRLSCA | A | | | | ADSVK | MAFDY |
| | SVSSAVAW | ASGFTFSSS | | | | | G | |
| | YQQKPGKA | SIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARGY | | | | | | |
| | | FYYGWW | | | | | | |
| | | AMAFDYW | | | | | | |
| | | GQGTLVT | | | | | | |
| | | VSS | | | | | | |
| AMRA3-17 | DIQMTQSPS | EVQLVESG | RASQS | SASSL | QQSQ | SSYIH | SISSSS | SYSYM |
| | SLSASVGDR | GGLVQPG | VSSAV | YS | WYEP | | GSTSY | SQWG |
| | VTITCRASQ | GSLRLSCA | A | | LIT | | ADSVK | WYQY |
| | SVSSAVAW | ASGFTIYSS | | | | | G | SGMDY |
| | YQQKPGKA | YIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSQWYE | AYLQMNS | | | | | | |
| | PLITFGQGT | LRAEDTAV | | | | | | |
| | KVEIKRTV | YYCARSYS | | | | | | |
| | | YMSQWG | | | | | | |
| | | WYQYSG | | | | | | |
| | | MDYWGQ | | | | | | |
| | | GTLVTVSS | | | | | | |
| AMR | DIQMTQSPS | EVQLVESG | RASQS | SASSL | QQGS | YSSIH | SISSSS | YAWW |
| A3-18 | SLSASVGDR | GGLVQPG | VSSAV | YS | YTYR | | GYTSY | AHGLD |
| | VTITCRASQ | GSLRLSCA | A | | LIT | | ADSVK | Y |
| | SVSSAVAW | ASGFTVSY | | | | | | |
| | YQQKPGKA | SSIHWVRQ | | | | | G | |
| | PKLLIYSAS | APGKGLE | | | | | | |
| | SLYSGVPSR | WVASISSS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQGSYTYR | TAYLQMN | | | | | | |
| | LITFGQGTK | SLRAEDTA | | | | | | |
| | VEIKRTV | VYYCARY | | | | | | |
| | | AWWAHG | | | | | | |
| | | LDYWGQG | | | | | | |
| | | TLVTVSS | | | | | | |
| AMRA3-21 | DIQMTQSPS | EVQLVESG | RASQS | SASSL | QQAS | SYSIH | SIYSSY | QYSMH |
| | SLSASVGDR | GGLVQPG | VSSAV | YS | YWYN | | GYTSY | FPWGY |
| | VTITCRASQ | GSLRLSCA | A | | LFT | | ADSVK | GMDY |
| | SVSSAVAW | ASGFTISSY | | | | | G | |
| | YQQKPGKA | SIHWVRQ | | | | | | |
| | PKLLIYSAS | APGKGLE | | | | | | |
| | SLYSGVPSR | WVASIYSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQASYWY | TAYLQMN | | | | | | |
| | NLFTFGQG | SLRAEDTA | | | | | | |
| | TKVEIKRTV | VYYCARQ | | | | | | |
| | | YSMHFPW | | | | | | |
| | | GYGMDY | | | | | | |
| | | WGQGTLV | | | | | | |
| | | TVSS | | | | | | |
| AMRA3-22 | DIQMTQSPS | EVQLVESG | RASQS | SASSL | QQSD | SSSIH | YIYSSS | PVNYY |
| | SLSASVGDR | GGLVQPG | VSSAV | YS | MPPIT | | GYTSY | YQGAL |
| | VTITCRASQ | GSLRLSCA | A | | | | ADSVK | DY |
| | SVSSAVAW | ASGFTFYS | | | | | G | |
| | YQQKPGKA | SSIHWVRQ | | | | | | |
| | PKLLIYSAS | APGKGLE | | | | | | |
| | SLYSGVPSR | WVAYIYSS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQSDMPPI | TAYLQMN | | | | | | |
| | TFGQGTKV | SLRAEDTA | | | | | | |
| | EIKRTV | VYYCARP | | | | | | |
| | | VNYYYQG | | | | | | |
| | | ALDYWGQ | | | | | | |
| | | GTLVTVSS | | | | | | |
| AMRA3-23 | DIQMTQSPS | EVQLVESG | RASQS | SASSL | QQYY | SSSIH | SISPSS | YHYMF |
| | SLSASVGDR | GGLVQPG | VSSAV | YS | VFPIT | | GYTYY | EYDKG |
| | VTITCRASQ | GSLRLSCA | A | | | | ADSVK | ESKWG |
| | SVSSAVAW | ASGFTVYS | | | | | G | YYGFD |
| | YQQKPGKA | SSIHWVRQ | | | | | | Y |
| | PKLLIYSAS | APGKGLE | | | | | | |
| | SLYSGVPSR | WVASISPS | | | | | | |
| | FSGSRSGTD | SGYTYYA | | | | | | |
| | FTLTISSLQ | DSVKGRFT | | | | | | |
| | PEDFATYYC | ISADTSKN | | | | | | |
| | QQYYVFPI | TAYLQMN | | | | | | |
| | TFGQGTKV | SLRAEDTA | | | | | | |
| | EIKRTV | VYYCARYH | | | | | | |
| | | YMFEYDKG | | | | | | |
| | | ESKWGYYG | | | | | | |
| | | FDYWGQGT | | | | | | |
| | | LVT | | | | | | |
| | | VSS | | | | | | |
| AMRA11-1 | DIQMTQSPS | EVQLVESG | RASQS | SASSL | QQSQ | YSSIH | SIYSYY | NSWS |
| | SLSASVGDR | GGLVQPG | VSSAV | YS | YFPIT | | GYTSY | WYSG |
| | VTITCRASQ | GSLRLSCA | A | | | | ADSVK | VGMD |
| | SVSSAVAW | ASGFTISY | | | | | G | Y |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSQYFPI | TAYLQMN | | | | | | |
| | TFGQG | SLRAEDTA | | | | | | |
| | TKVEI | VYYCARNS | | | | | | |
| | KRTV | WSWYSGVG | | | | | | |
| | | MDYWGQG | | | | | | |
| | | TLVTVSS | | | | | | |
| AMRA11- | DIQMTQSPS | EVQLVESG | RASQS | SASSL | QQSSS | SSSIH | SISSYS | YPYG |
| 2 | SLSASVGDR | GGLVQPG | VSSAV | YS | SLIT | | SSTYY | WGWG |
| | VTITCRASQ | GSLRLSCA | A | | | | ADSVK | GSGLD |
| | SVSSAVAW | ASGFTISSS | | | | | G | Y |
| | YQQKPGKA | SIHWVRQ | | | | | | |
| | PKLLIYSAS | APGKGLE | | | | | | |
| | SLYSGVPSR | WVASISSY | | | | | | |
| | FSGSRSGTD | SSSTYYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARYP | | | | | | |
| | | YGWGWG | | | | | | |
| | | GSGLDYW | | | | | | |
| | | GQGTLVT | | | | | | |
| | | VSS | | | | | | |
| AMRA11- | DIQMTQSPS | EVQLVESG | RASQS | SASSL | QQFD | YSSIH | SIYSYY | GEKW |
| 15 | SLSASVGDR | GGLVQPG | VSSAV | YS | FQYLI | | GSTYY | ALDY |
| | VTITCRASQ | GSLRLSCA | A | | T | | ADSVK | |
| | SVSSAVAW | ASGFTVYY | | | | | G | |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | YGSTYYA | | | | | | |
| | FTLTISSLQ | DSVKGRFT | | | | | | |
| | PEDFATYYC | ISADTSKN | | | | | | |
| | QQFDFQYL | TAYLQMN | | | | | | |
| | ITFGQGTKV | SLRAEDTA | | | | | | |
| | EIKRTV | VYYCARG | | | | | | |
| | | EKWALDY | | | | | | |
| | | WGQGTLV | | | | | | |
| | | TVSS | | | | | | |
| AMR | DIQMTQSPS | EVQLVESG | RASQS | SASSL | QQYM | YSSIH | SISSSS | EPYNY |
| A11- | SLSASVGDR | GGLVQPG | VSSAV | YS | YYQP | | GSTSY | NWYG |
| 16 | VTITCRASQ | GSLRLSCA | A | | LIT | | ADSVK | MDY |
| | SVSSAVAW | ASGFTVYY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQYMYYQ | AYLQMNS | | | | | | |
| | PLITFGQGT | LRAEDTAV | | | | | | |
| | KVEIKRTV | YYCAREP | | | | | | |
| | | YNYNWY | | | | | | |
| | | GMDYWGQ | | | | | | |
| | | GTLVTVSS | | | | | | |
| AMR | DIQMTQSPS | EVQLVESG | RASQS | SASSL | QQSL | SSSIH | SIYSYS | HGSYG |
| A311- | SLSASVGDR | GGLVQPG | VSSAV | YS | WWPI | | GYTSY | SWWA |
| 2 | VTITCRASQ | GSLRLSCA | A | | T | | ADSVK | LDY |
| | SVSSAVAW | ASGFTVSS | | | | | G | |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQSLWWPI | TAYLQMN | | | | | | |
| | TFGQGTKV | SLRAEDTA | | | | | | |
| | EIKRTV | VYYCARH | | | | | | |
| | | GSYGSWW | | | | | | |
| | | ALDYWGQ | | | | | | |
| | | GTLVTVSS | | | | | | |
| AMR | DIQMTQSPS | EVQLVESG | RASQS | SASSL | QQYF | SSSIH | SISSYY | ASYYS |
| A311- | SLSASVGDR | GGLVQPG | VSSAV | YS | YFPIT | | GSTSY | GYGSS |
| 10 | VTITCRASQ | GSLRLSCA | A | | | | ADSVK | YPYYM |
| | SVSSAVAW | ASGFTFYS | | | | | G | GLDY |
| | YQQKPGKA | SSIHWVRQ | | | | | | |
| | PKLLIYSAS | APGKGLE | | | | | | |
| | SLYSGVPSR | WVASISSY | | | | | | |
| | FSGSRSGTD | YGSTSYA | | | | | | |
| | FTLTISSLQ | DSVKGRFT | | | | | | |
| | PEDFATYYC | ISADTSKN | | | | | | |
| | QQYFYFPI | TAYLQMN | | | | | | |
| | TFGQGTKV | SLRAEDTA | | | | | | |
| | EIKRTV | VYYCARA | | | | | | |
| | | SYYSGYG | | | | | | |
| | | SSYPYYMG | | | | | | |
| | | LDYWGQG | | | | | | |
| | | TLVTVSS | | | | | | |
| AMR | DIQMTQSPS | EVQLVESG | RASQS | SASSL | QQGS | SYSIH | SISSSS | MNWS |
| A311- | SLSASVGDR | GGLVQPG | VSSAV | YS | YRNP | | GYTSY | HYAM |
| 14 | VTITCRASQ | GSLRLSCA | A | | LLT | | ADSVK | DY |
| | SVSSAVAW | ASGFTFSS | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQGSYRNP | TAYLQMN | | | | | | |
| | LLTFGQGT | SLRAEDTA | | | | | | |
| | KVEIKRTV | VYYCARM | | | | | | |
| | | NWSHYA | | | | | | |
| | | MDYWGQ | | | | | | |
| | | GTLVTVSS | | | | | | |
| AMR | DIQMTQSPS | EVQLVESG | RASQS | SASSL | QQSSS | SSSIH | YISSYS | YWYG |
| A311- | SLSASVGDR | GGLVQPG | VSSAV | YS | SLIT | | GYTYY | HYHSY |
| 16 | VTITCRASQ | GSLRLSCA | A | | | | ADSVK | FGLDY |
| | SVSSAVAW | ASGFTISS | | | | | G | |
| | YQQKPGKA | SSIHWVRQ | | | | | | |
| | PKLLIYSAS | APGKGLE | | | | | | |
| | SLYSGVPSR | WVAYISSY | | | | | | |
| | FSGSRSGTD | SGYTYYA | | | | | | |
| | FTLTISSLQ | DSVKGRFT | | | | | | |
| | PEDFATYYC | ISADTSKN | | | | | | |
| | QQSSSSLI | TAYLQMN | | | | | | |
| | TFGQGTKV | SLRAEDTA | | | | | | |
| | EIKRTV | VYYCARY | | | | | | |
| | | WYGHYHSY | | | | | | |
| | | FGLDYWGQ | | | | | | |
| | | GTLVTVSS | | | | | | |
| AMR | DIQMTQSPS | EVQLVESG | RASQS | SASSL | QQSSS | SSSIH | SISSYS | YPYGS |
| A311- | SLSASVGDR | GGLVQPGG | VSSAV | YS | SLIT | | GSTYY | HVYTG |
| 17 | VTITCRASQ | SLRLSCAA | A | | | | ADSVK | LDY |
| | SVSSAVAW | SGFTISSS | | | | | G | |
| | YQQKPGKA | SIHWVRQ | | | | | | |
| | PKLLIYSAS | APGKGLE | | | | | | |
| | SLYSGVPSR | WVASISSY | | | | | | |
| | FSGSRSGTD | SGSTYYA | | | | | | |
| | FTLTISSLQ | DSVKGRFT | | | | | | |
| | PEDFATYYC | ISADTSKN | | | | | | |
| | QQSSSSLIT | TAYLQMN | | | | | | |
| | FGQGTKVEI | SLRAEDTA | | | | | | |
| | KRTV | VYYCARY | | | | | | |
| | | PYGSHVY | | | | | | |
| | | TGLDYWG | | | | | | |
| | | QGTLVTVSS | | | | | | |
| AMR | DIQMTQSPS | EVQLVESG | RASQS | SASSL | QQWN | SSSIH | SIYSSS | VYSSR |
| A311- | SLSASVGDR | GGLVQPG | VSSAV | YS | WADY | | GSTSY | YWGW |
| 18 | VTITCRASQ | GSLRLSCA | A | | LVT | | ADSVK | GVAFD |
| | SVSSAVAW | ASGFTISS | | | | | G | Y |
| | YQQKPGKA | SSIHWVRQ | | | | | | |
| | PKLLIYSAS | APGKGLE | | | | | | |
| | SLYSGVPSR | WVASIYSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQWNWAD | AYLQMNS | | | | | | |
| | YLVTFGQG | LRAEDTAV | | | | | | |
| | TKVEIKRTV | YYCARVY | | | | | | |
| | | SSRYWGW | | | | | | |
| | | GVAFDYW | | | | | | |
| | | GQGTLVT | | | | | | |
| | | VSS | | | | | | |
| AMR | DIQMTQSPS | EVQLVESG | RASQSV | SASS | QQYYW | SSSIH | YIYSSS | RSFPQ |
| A311- | SLSASVGDR | GGLVQPG | SSAV | LYS | YSLIT | | GYTSY | WYNG |
| 19 | VTITCRASQ | GSLRLSCA | A | | | | ADSVK | SYTPW |
| | SVSSAVAW | ASGFTVYS | | | | | G | PAMDY |
| | YQQKPGKA | SSIHWVRQ | | | | | | |
| | PKLLIYSAS | APGKGLE | | | | | | |
| | SLYSGVPSR | WVAYIYSS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQYYWYS | TAYLQMN | | | | | | |
| | LITFGQGTK | SLRAEDTA | | | | | | |
| | VEIKRTV | VYYCARRS | | | | | | |
| | | FPQWYNGSY | | | | | | |
| | | TPWPAMDYW | | | | | | |
| | | GQGTLVTVSS | | | | | | |
| AMR | DIQMTQSPS | EVQLVESG | RASQSV | SASS | QQYMW | SSSIH | SIYSYS | PFYWG |
| A311- | SLSASVGDR | GGLVQPG | SSAV | LYS | WPVT | | SYTSY | ERYAL |
| 20 | VTITCRASQ | GSLRLSCA | A | | | | ADSVK | DY |
| | SVSSAVAW | ASGFTVSS | | | | | G | |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | SSYTSYAD | | | | | | |
| | FTLTISSLQ | SVKGRFTI | | | | | | |
| | EPDFATYYC | SADTSKNT | | | | | | |
| | QQYMWWP | AYLQMNS | | | | | | |
| | VTFGQGTK | LRAEDTAV | | | | | | |
| | VEIKRTV | YYCARPF | | | | | | |
| | | YWGERYA | | | | | | |
| | | LDYWGQG | | | | | | |
| | | TLVTVSS | | | | | | |
| AMR | DIQMTQSPS | EVQLVESG | RASQS | SASSL | QQSY | SSSIH | SIYSSY | IYGWS |
| A3-5 | SLSASVGDR | GGLVQPG | VSSAV | YS | STLVT | | GSTSY | YQGW |
| | VTITCRASQ | GSLRLSCA | A | | | | ADSVK | AGMD |
| | SVSSAVAW | ASGFTFYS | | | | | G | Y |
| | YQQKPGKA | SSIHWVRQ | | | | | | |
| | PKLLIYSAS | APGKGLE | | | | | | |
| | SLYSGVPSR | WVASIYSS | | | | | | |
| | FSGSRSGTD | YGSTSYA | | | | | | |
| | FTLTISSLQ | DSVKGRFT | | | | | | |
| | PEDFATYYC | ISADTSKN | | | | | | |
| | QQSYSTLV | TAYLQMN | | | | | | |
| | TFGQGTKV | SLRAEDTA | | | | | | |
| | EIKRTV | VYYCARIY | | | | | | |
| | | GWSYQGWA | | | | | | |
| | | GMDYWGQG | | | | | | |
| | | TLVTVSS | | | | | | |
| AMR | DIQMTQSPS | EVQLVESG | RASQS | SASSL | QQSSS | YSSIH | SIYPYY | GGDYY |
| A3-6 | SLSASVGDR | GGLVQPG | VSSAV | YS | SLIT | | GSTSY | WGWY |
| | VTITCRASQ | GSLRLSCA | A | | | | ADSVK | WVAM |
| | SVSSAVAW | ASGFTISY | | | | | G | DY |
| | YQQKPGKA | SSIHWVRQ | | | | | | |
| | PKLLIYSAS | APGKGLE | | | | | | |
| | SLYSGVPSR | WVASIYPY | | | | | | |
| | FSGSRSGTD | YGSTSYA | | | | | | |
| | FTLTISSLQ | DSVKGRFT | | | | | | |
| | PEDFATYYC | ISADTSKN | | | | | | |
| | QQSSSSLIT | TAYLQMN | | | | | | |
| | FGQGTKVEI | SLRAEDTA | | | | | | |
| | KRTV | VYYCARG | | | | | | |
| | | GDYYWG | | | | | | |
| | | WYWVAM | | | | | | |
| | | DYWGQGT | | | | | | |
| | | LVTVSS | | | | | | |
| AMR | DIQMTQSPS | EVQLVESG | RASQS | SASSL | QKSSS | SSYIH | SISSSY | MYYY |
| A3-9 | SLSASVGDR | GGLVQPG | VSSAV | YS | SLIT | | GSTSY | TYPGM |
| | VTITCRASQ | GSLRLSCA | A | | | | ADSVK | DY |
| | SVSSAVAW | ASGFTFSSS | | | | | G | |
| | YQQKPGKA | YIHWVRQ | | | | | | |
| | PKLLIYSAS | APGKGLE | | | | | | |
| | SLYSGVPSR | WVASISSS | | | | | | |
| | FSGSRSGTD | YGSTSYA | | | | | | |
| | FTLTIXSLQ | DSVKGRFT | | | | | | |
| | PEDFATYY | ISADTSKN | | | | | | |
| | CQKSSSSLI | TAYLQMN | | | | | | |
| | TFGQGTKV | SLRAEDTA | | | | | | |
| | EIKRTV | VYYCARM | | | | | | |
| | | YYYTYPG | | | | | | |
| | | MDYWGQ | | | | | | |
| | | GTLVTVSS | | | | | | |
| AMR | DIQMTQSPS | EVQLVESG | RASQS | SASSL | QKGS | SYSIH | SISPSS | YHYGG |
| A3-10 | SLSASVGDR | GGLVQPG | VSSAV | YS | SYLLT | | GYTSY | WSHY |
| | VTITCRASQ | GSLRLSCA | A | | | | ADSVK | MSGM |
| | SVSSAVAW | ASGFTIYS | | | | | G | DY |
| | YQQKPGKA | YSIHWVR | | | | | | |
| | PKLLIYSAS | QAPGKGLE | | | | | | |
| | SLYSGVPSR | WVASISPS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QKGSSYLL | TAYLQMN | | | | | | |
| | TFGQGTKV | SLRAEDTA | | | | | | |
| | EIKRTV | VYYCARY | | | | | | |
| | | HYGGWSH | | | | | | |
| | | YMSGMDYW | | | | | | |
| | | GQGTLVTV | | | | | | |
| | | SS | | | | | | |
| AMR | DIQMTQSPS | EVQLVESG | RASQS | SASSL | QQNY | YSSIH | SISSSY | GRYGG |
| A3-13 | SLSASVGDR | GGLVQPG | VSSAV | YS | YYHK | | GYTSY | MDY |
| | VTITCRASQ | GSLRLSCA | A | | LIT | | ADSVK | |
| | SVSSAVAW | ASGFTFSY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQNYYYH | TAYLQMN | | | | | | |
| | KLITFGQGT | SLRAEDTA | | | | | | |
| | KVEIKRTV | VYYCARGR | | | | | | |
| | | YGGMDYWG | | | | | | |
| | | QGTLV | | | | | | |
| | | TVSS | | | | | | |
| AMR | DIQMTQSPS | EVQLVESG | RASQS | SASSL | QQLS | SSSIH | SISSSY | GWYK |
| A3-19 | SLSASVGDR | GGLVQPG | VSSAV | YS | YVYK | | GYTSY | AMDY |
| | VTITCRASQ | GSLRLSCA | A | | LIT | | ADSVK | |
| | SVSSAVAW | ASGFTFYS | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQLSYVYK | TAYLQMN | | | | | | |
| | LITFGQGTK | SLRAEDTA | | | | | | |
| | VEIKRTV | VYYCARG | | | | | | |
| | | WYKAMD | | | | | | |
| | | YWGQGTL | | | | | | |
| | | VTVSS | | | | | | |
| AMR | DIQMTQSPS | EVQLVESG | RASQS | SASSL | QQSSS | YSSIH | SISSSY | MYYY |
| A3-24 | SLSASVGDR | GGLVQPG | VSSAV | YS | SLIT | | GSTYY | YYPGI |
| | VTITCRASQ | GSLRLSCA | A | | | | ADSVK | DY |
| | SVSSAVAW | ASGFTFSY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YGSTYYA | | | | | | |
| | FTLTISSLQ | DSVKGRFT | | | | | | |
| | PEDFATYYC | ISADTSKN | | | | | | |
| | QQSSSSLIT | TAYLQMN | | | | | | |
| | FGQGTKVEI | SLRAEDTA | | | | | | |
| | KRTV | VYYCARM | | | | | | |
| | | YYYYYPGI | | | | | | |
| | | DYWGQG | | | | | | |
| | | TLVTVSS | | | | | | |
| AMR | DIQMTQSPS | EVQLVESG | RASQS | SASSL | QQYY | SSSIH | SISPYY | SPYYW |
| A11-3 | SLSASVGDR | GGLVQPG | VSSAV | YS | YFPIT | | GYTSY | YQYFY |
| | VTITCRASQ | GSLRLSCA | A | | | | ADSVK | GWGL |
| | SVSSAVAW | ASGFTFSS | | | | | G | DY |
| | YQQKPGKA | SSIHWVRQ | | | | | | |
| | PKLLIYSAS | APGKGLE | | | | | | |
| | SLYSGVPSR | WVASISPY | | | | | | |
| | FSGSRSGTD | YGYTSYA | | | | | | |
| | FTLTISSLQ | DSVKGRFT | | | | | | |
| | PEDLATYYC | ISADTSKN | | | | | | |
| | QQYYYFPI | TAYLQMN | | | | | | |
| | TFGQGTKV | SLRAEDTA | | | | | | |
| | EIKRTV | VYYCARSP | | | | | | |
| | | YYWYQYFY | | | | | | |
| | | GWGLDYWG | | | | | | |
| | | QGTLVTVSS | | | | | | |
| AMR | DIQMTQSPS | EVQLVESG | RASQS | SASSL | QQSSS | YSSIH | SISSSS | SPYW |
| A11-4 | SLSASVGDR | GGLVQPG | VSSAV | YS | SLIT | | GSTSY | WNYM |
| | VTITCRASQ | GSLRLSCA | A | | | | ADSVK | SAMDY |
| | SVSSAVAW | ASGFTFSY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARSPY | | | | | | |
| | | WWNYMS | | | | | | |
| | | AMDYWG | | | | | | |
| | | QGTLVTV | | | | | | |
| | | SS | | | | | | |
| AMR | DIQMTQSPS | EVQLVESG | RASQS | SASSL | QQGW | SYSIH | SISPYY | WSWQ |
| A11-7 | SLSASVGDR | GGLVQPG | VSSAV | YS | WWPF | | GYTSY | YYSGH |
| | VTITCRASQ | GSLRLSCA | A | | T | | ADSVK | SSWGL |
| | SVSSAVAW | ASGFTVSS | | | | | G | DY |
| | YQQKPGKA | YSIHWVR | | | | | | |
| | PKLLIYSAS | QAPGKGLE | | | | | | |
| | SLYSGVPSR | WVASISPY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQGWWW | TAYLQMN | | | | | | |
| | PFTFGQGT | SLRAEDTA | | | | | | |
| | KVEIKRTV | VYYCARW | | | | | | |
| | | SWQYYSGH | | | | | | |
| | | SSWGLDYW | | | | | | |
| | | GQGTLVTV | | | | | | |
| | | SS | | | | | | |
| AMR | DIQMTQSPS | EVQLVESG | RASQS | SASSL | QQSW | SSSIH | SIYSYY | WYNE |
| A11-8 | SLSASVGDR | GGLVQPG | VSSAV | YS | YFPLT | | GYTSY | YYHDY |
| | VTITCRASQ | GSLRLSCA | A | | | | ADSVK | YWDA |
| | SVSSAVAW | ASGFTVSS | | | | | G | MDY |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSWYFPL | TAYLQMN | | | | | | |
| | TFGQGTKV | SLRAEDTA | | | | | | |
| | EIKRTV | VYYCARW | | | | | | |
| | | YNEYYHD | | | | | | |
| | | YYWDAM | | | | | | |
| | | DYWGQGT | | | | | | |
| | | LVTVSS | | | | | | |
| AMR | DIQMTQSPS | EVQLVESG | RASQS | SASSL | QQSSS | YSSIH | SISSSS | WMYW |
| A11-9 | SLSASVGDR | GGLVQPG | VSSAV | YS | SLIT | | GSTSY | WSFAL |
| | VTITCRASQ | GSLRLSCA | A | | | | ADSVK | DY |
| | SVSSAVAW | ASGFTLYY | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARW | | | | | | |
| | | MYWWSFA | | | | | | |
| | | LDYWGQ | | | | | | |
| | | GTLVTVSS | | | | | | |
| AMR | DIQMTQSPS | EVQLVESG | RASQS | SASSL | QQSY | SSSIH | SIYSYY | WQYH |
| A11- | SLSASVGDR | GGLVQPG | VSSAV | YS | LWPI | | GYTSY | YNYW |
| 10 | VTITCRASQ | GSLRLSCA | A | | T | | ADSVK | YGMD |
| | SVSSAVAW | ASGFTVSS | | | | | G | Y |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSYLWPI | TAYLQMN | | | | | | |
| | TFGQGTKV | SLRAEDTA | | | | | | |
| | EIKRTV | VYYCARW | | | | | | |
| | | QYHYNY | | | | | | |
| | | WYGMDY | | | | | | |
| | | WGQGTL | | | | | | |
| | | VTVSS | | | | | | |
| AMR | DIQMTQSPS | EVQLVESG | RASQS | SASSL | QQYP | YSSIH | SISPYS | GYDYY |
| A11- | SLSASVGDR | GGLVQPG | VSSAV | YS | MSLIT | | GYTSY | AGLDY |
| 11 | VTITCRASQ | GSLRLSCA | A | | | | ADSVK | |
| | SVSSAVAW | ASGFTVSY | | | | | G | |
| | YQQKPGKA | SSIHWVRQ | | | | | | |
| | PKLLIYSAS | APGKGLE | | | | | | |
| | SLYSGVPSR | WVASISPY | | | | | | |
| | FSGSRSGTD | SGYTSYA | | | | | | |
| | FTLTISSLQ | DSVKGRFT | | | | | | |
| | PEDFATYY | ISADTSK | | | | | | |
| | CQQYPMSLI | NTAYLQMN | | | | | | |
| | TFGQGTKV | SLRAEDTA | | | | | | |
| | EIKRTV | VYYCARG | | | | | | |
| | | YDYYAGLD | | | | | | |
| | | YWGQGTLV | | | | | | |
| | | TVSS | | | | | | |
| AMR | DIQMTQSPS | EVQLVESG | RASQS | SASSL | QQYY | YYSIH | SISPYY | WESEY |
| A11- | SLSASVGDR | GGLVQPG | VSSAV | YS | YFPIT | | GYTSY | SGTYE |
| 12 | VTITCRASQ | GSLRLSCA | A | | | | ADSVK | DY |
| | SVSSAVAW | ASGFTFSY | | | | | G | |
| | YQQKPGKA | YSIHWVR | | | | | | |
| | PKLLIYSAS | QAPGKGLE | | | | | | |
| | SLYSGVPSR | WVASISPY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQYYYFPI | TAYLQMN | | | | | | |
| | TFGQGTKV | SLRAEDTA | | | | | | |
| | EIKRTV | VYYCARW | | | | | | |
| | | ESEYSGTY | | | | | | |
| | | EDYWAGMD | | | | | | |
| | | YWGQGTLV | | | | | | |
| | | TVSS | | | | | | |
| AMR | DIQMTQSPS | EVQLVESG | RASQS | SASSL | QQYM | YSSIH | SISSSY | TGYW |
| A11- | SLSASVGDR | GGLVQPG | VSSAV | YS | WWPI | | SYTSY | QGYLA |
| 13 | VTITCRASQ | GSLRLSCA | A | | T | | ADSVK | LDY |
| | SVSSAVAW | ASGFTISYS | | | | | G | |
| | YQQKPGKA | SIHWVRQ | | | | | | |
| | PKLLIYSAS | APGKGLE | | | | | | |
| | SLYSGVPSR | WVASISSS | | | | | | |
| | FSGSRSGTD | YSYTSYAD | | | | | | |
| | FTLTISSLQ | SVKGRFTI | | | | | | |
| | PEDFATYYC | SADTSKNT | | | | | | |
| | QQYMWWP | AYLQMNS | | | | | | |
| | ITFGQGTKV | LRAEDTAV | | | | | | |
| | EIKRTV | YYCARTG | | | | | | |
| | | YWQGYLA | | | | | | |
| | | LDYWGQ | | | | | | |
| | | GTLVTVSS | | | | | | |
| AMR | DIQMTQSPS | EVQLVESG | RASQS | SAS | QQSSS | YSSIH | SISSSS | TYYYY |
| A11-14 | SLSASVGDR | GGLVQPG | VSSAV | SLYS | SLIT | | GSTSY | WNSTP |
| | VTITCRASQ | GSLRLSCA | A | | | | ADSVK | AMDY |
| | SVSSAVAW | ASGFTISYS | | | | | G | |
| | YQQKPGKA | SIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARTY | | | | | | |
| | | YYYWNST | | | | | | |
| | | PAMDYWG | | | | | | |
| | | QGTLVTVSS | | | | | | |
| AMR | DIQMTQSPS | EVQLVESG | RASQ | SASS | QQSY | SSSIH | SISSSY | WYNSS |
| A11-18 | SLSASVGDR | GGLVQPG | SVSSAV | LYS | GYPVT | | GYTSY | WYYS |
| | VTITCRASQ | GSLRLSCA | A | | | | ADSVK | NWWY |
| | SVSSAVAW | ASGFTFSS | | | | | G | KGFGM |
| | YQQKPGKA | SSIHWVRQ | | | | | | DY |
| | PKLLIYSAS | APGKGLE | | | | | | |
| | SLYSGVPSR | WVASISSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSYGYPV | TAYLQMN | | | | | | |
| | TFGQGTKV | SLRAEDTA | | | | | | |
| | EIKRTV | VYYCARWY | | | | | | |
| | | NSSWYYSN | | | | | | |
| | | WWYKGFGMD | | | | | | |
| | | YWGQGTLVT | | | | | | |
| | | VSS | | | | | | |
| AMR | DIQMTQSPS | EVQLVESG | RASQS | SASSL | QQYY | SSSIH | SISSSY | TSYTY |
| A11- | SLSASVGDR | GGLVQPG | VSSAV | YS | SSLFT | | GYTSY | PVYTY |
| 20 | VTITCRASQ | GSLRLSCA | A | | | | ADSVK | YGFDY |
| | SVSSAVAW | ASGFTFYS | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQYYSSLF | TAYLQMN | | | | | | |
| | TFGQGTKV | SLRAEDTA | | | | | | |
| | EIKRTV | VYYCART | | | | | | |
| | | SYTYPVYT | | | | | | |
| | | YYGFDYWG | | | | | | |
| | | QGTLVT | | | | | | |
| | | VSS | | | | | | |
| AMR | DIQMTQSPS | EVQLVESG | RASQS | SASSL | QQSW | SSSIH | SISSSY | YRYSS |
| A11- | SLSASVGDR | GGLVQPG | VSSAV | YS | YYPLT | | GYTSY | WNRG |
| 22 | VTITCRASQ | GSLRLSCA | A | | | | ADSVK | AIDY |
| | SVSSAVAW | ASGFTLYS | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSWYYPL | TAYLQMN | | | | | | |
| | TFGQGTKV | SLRAEDTA | | | | | | |
| | EIKRTV | VYYCARY | | | | | | |
| | | RYSSWNR | | | | | | |
| | | GAIDYWG | | | | | | |
| | | QGTLVTV | | | | | | |
| | | SS | | | | | | |
| AMR | DIQMTQSPS | EVQLVESG | RASQS | SASSL | QQSY | SSSIH | SIYSYY | WSKSP |
| A11- | SLSASVGDR | GGLVQPG | VSSAV | YS | WWPL | | GYTSY | WYYQ |
| 24 | VTITCRASQ | GSLRLSCA | A | | T | | ADSVK | GIDY |
| | SVSSAVAW | ASGFTVSS | | | | | G | |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSYWWP | TAYLQMN | | | | | | |
| | LTFGQGTK | SLRAEDTA | | | | | | |
| | VEIKRTV | VYYCARW | | | | | | |
| | | SKSPWYYQ | | | | | | |
| | | GIDYWGQG | | | | | | |
| | | TLVTVSS | | | | | | |
| IBA1-4 | DIQMTQSPS | EVQLVESG | RASQS | SASSL | QQYH | SSSIH | SIYSYS | QYSSS |
| | SLSASVGDR | GGLVQPG | VSSAV | YS | YWAS | | GSTYY | YYVW |
| | VTITCRASQ | GSLRLSCA | A | | LIT | | ADSVK | PGMDY |
| | SVSSAVAW | ASGFTVSS | | | | | G | |
| | YQQKPGKA | SSIHWVRQ | | | | | | |
| | PKLLIYSAS | APGKGLE | | | | | | |
| | SLYSGVPSR | WVASIYSY | | | | | | |
| | FSGSRSGTD | SGSTYYA | | | | | | |
| | FTLTISSLQ | DSVKGRFT | | | | | | |
| | PEDFATYYC | ISADTSKN | | | | | | |
| | QQYHYWA | TAYLQMN | | | | | | |
| | SLITFGQGT | SLRAEDTA | | | | | | |
| | KVEIKRTV | VYYCARQ | | | | | | |
| | | YSSSYYV | | | | | | |
| | | WPGMDY | | | | | | |
| | | WGQGTLV | | | | | | |
| | | TVSS | | | | | | |
| IBA1-7 | DIQMTQSPS | EVQLVESG | RASQS | SASSL | QQSY | SSSIH | SISSYY | MHYS |
| | SLSASVGDR | GGLVQPG | VSSAV | YS | WWKS | | GSTSY | WQEY |
| | VTITCRASQ | GSLRLSCA | A | | LVT | | ADSVK | YSYD |
| | SVSSAVAW | ASGFTLSS | | | | | G | WGMD |
| | YQQKPGKA | SSIHWVRQ | | | | | | Y |
| | PKLLIYSAS | APGKGLE | | | | | | |
| | SLYSGVPSR | WVASISSY | | | | | | |
| | FSGSRSGTD | YGSTSYA | | | | | | |
| | FTLTISSL | DSVKGRF | | | | | | |
| | QPEDFATY | TISADTS | | | | | | |
| | YCQQSYWWK | KNTAYLQ | | | | | | |
| | SLVTFGQG | MNSLRAE | | | | | | |
| | TKVEIKRTV | DTAVYYCA | | | | | | |
| | | RMHYSWQE | | | | | | |
| | | YYSYDWGM | | | | | | |
| | | DYWGQGTL | | | | | | |
| | | VTVSS | | | | | | |
| IBA1-8 | DIQMTQSPS | EVQLVESG | RASQS | SASSLYS | QQPY | YSSIH | SIYPSY | WQGY |
| | SLSASVGDR | GGLVQPG | VSSAV | | YPLIT | | GSTSY | YQPAL |
| | VTITCRASQ | GSLRLSCA | A | | | | ADSVK | DY |
| | SVSSAVAW | ASGFTISY | | | | | G | |
| | YQQKPGKA | SSIHWVRQ | | | | | | |
| | PKLLIYSAS | APGKGLE | | | | | | |
| | SLYSGVPSR | WVASIYPS | | | | | | |
| | FSGSRSGTD | YGSTSYA | | | | | | |
| | FTLTISSLQ | DSVKGRFT | | | | | | |
| | PEDFATYYC | ISADTSKN | | | | | | |
| | QQPYYPLI | TAYLQMN | | | | | | |
| | TFGQGTKV | SLRAEDTA | | | | | | |
| | EIKRTV | VYYCARW | | | | | | |
| | | QGYYQPA | | | | | | |
| | | LDYWGQG | | | | | | |
| | | TLVTVSS | | | | | | |
| IBA1-12 | DIQMTQSPS | EVQLVESG | RASQS | SASSL | QQSS | SSSIH | SISPYY | WGYG |
| | SLSASVGDR | GGLVQPG | VSSAV | YS | KYYY | | GYTSY | WYWY |
| | VTITCRASQ | GSLRLSCA | A | | PIT | | ADSVK | GLDY |
| | SVSSAVAW | ASGFTISS | | | | | G | |
| | YQQKPGKA | SSIHWVRQ | | | | | | |
| | PKLLIYSAS | APGKGLE | | | | | | |
| | SLYSGVPSR | WVASISPY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSSKYYY | TAYLQMN | | | | | | |
| | PITFGQGTK | SLRAEDTA | | | | | | |
| | VEIKRTV | VYYCARW | | | | | | |
| | | GYGWYW | | | | | | |
| | | YGLDYWG | | | | | | |
| | | QGTLVTVS | | | | | | |
| | | S | | | | | | |
| IBA1-13 | DIQMTQSPS | EVQLVESG | RASQS | SASSL | QQGH | SSSIH | SIYSSY | WYGG |
| | SLSASVGDR | GGLVQPG | VSSAV | YS | DMNP | | GYTYY | MDY |
| | VTITCRASQ | GSLRLSCA | A | | VT | | ADSVK | |
| | SVSSAVAW | ASGFTLSS | | | | | G | |
| | YQQKPGKA | SSIHWVRQ | | | | | | |
| | PKLLIYSAS | APGKGLE | | | | | | |
| | SLYSGVPSR | WVASIYSS | | | | | | |
| | FSGSRSGTD | YGYTYYA | | | | | | |
| | FTLTISSLQ | DSVKGRFT | | | | | | |
| | PEDFATYYC | ISADTSKN | | | | | | |
| | QQGHDMN | TAYLQMN | | | | | | |
| | PVTFGQGT | SLRAEDTA | | | | | | |
| | KVEIKRTV | VYYCARY | | | | | | |
| | | YYYWYG | | | | | | |
| | | GMDYWGQ | | | | | | |
| | | GTLVTVS | | | | | | |
| | | S | | | | | | |
| IBA1-19 | DIQMTQSPS | EVQLVESG | RASQS | SASSL | QQSW | YSSIH | SIYPSS | GWWY |
| | SLSASVGDR | GGLVQPG | VSSAV | YS | MSDS | | GYTSY | WMAW |
| | VTITCRASQ | GSLRLSCA | A | | LIT | | ADSVK | DYAM |
| | SVSSAVAW | ASGFTFSY | | | | | G | DY |
| | YQQKPGKA | SSIHWVRQ | | | | | | |
| | PKLLIYSAS | APGKGLE | | | | | | |
| | SLYSGVPSR | WVASIYPS | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQSWMSD | TAYLQMN | | | | | | |
| | SLITFGQGT | SLRAEDTA | | | | | | |
| | KVEIKRTV | VYYCARG | | | | | | |
| | | WWYWMA | | | | | | |
| | | WDYAMD | | | | | | |
| | | YWGQGTL | | | | | | |
| | | VTVSS | | | | | | |
| IBA1- | DIQMTQSPS | EVQLVESG | RASQS | SASSL | QQMQ | SSSIH | SISSYY | YYSYS |
| 21 | SLSASVGDR | GGLVQPGG | VSSAV | YS | YSGW | | GYTSY | SGYGY |
| | VTITCRASQ | SLRLSCAA | A | | LIT | | ADSVK | YDYFD |
| | SVSSAVAW | SGFTISSS | | | | | G | WGAM |
| | YQQKPGKA | SIHWVRQ | | | | | | DY |
| | PKLLIYSAS | APGKGLE | | | | | | |
| | SLYSGVPSR | WVASISSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQMQYSG | TAYLQMN | | | | | | |
| | WLITFGQG | SLRAEDTA | | | | | | |
| | TKVEIKRTV | VYYCARY | | | | | | |
| | | YSYSSGY | | | | | | |
| | | GYYDYFD | | | | | | |
| | | WGAMDYW | | | | | | |
| | | GQGTLVT | | | | | | |
| | | VSS | | | | | | |
| UCHT | DIQMTQTTS | EVQLQQSG | RASQD | YTSRL | QQGN | GYTMN | LINPYK | SGYYG |
| 1 | SLSASLGDR | PELVKPGA | IRNYL | HS | TLPW | | GVSTY | DSDW |
| | VTISCRASQ | SMKISCKA | N | | T | | NQKFK | YFDV |
| | DIRNYLNW | SGYSFTGY | | | | | D | |
| | YQQKPDGT | TMNWVKQ | | | | | | |
| | VKLLIYYTS | SHGKNLE | | | | | | |
| | RLHSGVPSK | WMGLINP | | | | | | |
| | FSGSGSGTD | YKGVSTY | | | | | | |
| | YSLTISNLE | NQKFKDK | | | | | | |
| | QEDIATYFC | ATLTVDKS | | | | | | |
| | QQGNTLPW | SSTAYMEL | | | | | | |
| | TFAGGTKL | LSLTSEDS | | | | | | |
| | EIK | AVYYCAR | | | | | | |
| | | SGYYGDS | | | | | | |
| | | DWYFDVWG | | | | | | |
| | | QGTTLTV | | | | | | |
| | | SS | | | | | | |
| 12C- | DIQMTQSPS | EVQLVESG | RASQS | SASSL | QQDW | FTFSSY | SISPSS | YGGRS |
| ARS- | SLSASVGDR | GGLVQPG | VSSAV | YS | YFPIT | YIH | GSTYY | YWQK |
| Fab59 | VTITCRASQ | GSLRLSCA | A | | | | ADSVK | QDSYF |
| | SVSSAVAW | ASGFTFSS | | | | | G | YQHGL |
| | YQQKPGKA | YYIHWVR | | | | | | DY |
| | PKLLIYSAS | QAPGKGLE | | | | | | |
| | SLYSGVPSR | WVASISPS | | | | | | |
| | FSGSRSGTD | SGSTYYA | | | | | | |
| | FTLTISSLQ | DSVKGRFT | | | | | | |
| | PEDFATYYC | ISADTSKN | | | | | | |
| | QQDWYFPI | TAYLQMN | | | | | | |
| | TFGQGTKV | SLRAEDTA | | | | | | |
| | EIK | VYYCARY | | | | | | |
| | | GGRSYWQ | | | | | | |
| | | KQDSYFY | | | | | | |
| | | QHGLDYW | | | | | | |
| | | GQGTLVSS | | | | | | |
| 12C- | DIQMTQSPS | EVQLVESG | RASQS | SASSL | QQSSS | FTFSSS | SISSYS | SYSYS |
| ARS- | SLSASVGDR | GGLVQPG | VSSAV | YS | SLIT | SIH | GYTSY | EFRYY |
| Fab56 | VTITCRASQ | GSLRLSCA | A | | | | ADSVK | YSGQG |
| | SVSSAVAW | ASGFTFSS | | | | | G | MDY |
| | YQQKPGKA | SSIHWVRQ | | | | | | |
| | PKLLIYSAS | APGKGLE | | | | | | |
| | SLYSGVPSR | WVASISSY | | | | | | |
| | FSGSRSGTD | SGYTSYA | | | | | | |
| | FTLTISSLQ | DSVKGRFT | | | | | | |
| | PEDFATYYC | ISADTSKN | | | | | | |
| | QQSSSSLIT | TAYLQMN | | | | | | |
| | FGQGTKVEI | SLRAEDTA | | | | | | |
| | K | VYYCARS | | | | | | |
| | | YSYSEFRY | | | | | | |
| | | YYSGQGMDY | | | | | | |
| | | WGQGTLVSS | | | | | | |
| 12C- | DIQMTQSPS | EVQLVESG | RASQS | SASSL | QQSSS | FTFSSS | SISSSS | SNYG |
| ARS- | SLSASVGDR | GGLVQPG | VSSAV | YS | SLIT | SIH | GSTSY | WRWH |
| Fab30 | VTITCRASQ | GSLRLSCA | A | | | | ADSVK | LVGM |
| | SVSSAVAW | ASGFTFSS | | | | | G | DY |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNSL | | | | | | |
| | FGQGTKVEI | RAEDTAVY | | | | | | |
| | K | YCARSNYG | | | | | | |
| | | WRWHLVGM | | | | | | |
| | | DYWGQGTL | | | | | | |
| | | VTVSS | | | | | | |
| 12C- | DIQMTQSPS | EVQLVESG | RASQS | SASSL | QQSSS | TFSSSS | SISSSS | SPYVY |
| ARS- | SLSASVGDR | GGLVQPG | VSSAV | YS | SLIT | IH | GSTSY | YWYM |
| Fab85 | VTITCRASQ | GSLRLSCA | A | | | | ADSVK | VGFDY |
| | SVSSAVAW | ASGFTFSS | | | | | G | |
| | YQQKPGKA | SSIHWVRQ | | | | | | |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | K | YYCARSPY | | | | | | |
| | | VYYWYMVG | | | | | | |
| | | FDYWGQGT | | | | | | |
| | | LVTVSS | | | | | | |
| P2AM | QSVLIQPRS | QVQLVQSE | TGTSS | DVSK | GSYA | TDAIT | GIIPLL | WSSVD |
| R-1 | VSGSPGQSV | PEVKKPGS | DVGGY | RPS | DTDTI | | DSVDY | TGLDY |
| | TISCTGTSS | SVKLSCKA | NYVS | | V | | AQRFQ | |
| | DVGGYNY | SGGTFSTD | | | | | G | |
| | VSWYQQHP | AITWVRQ | | | | | | |
| | GKAPKLMI | APGQGLEY | | | | | | |
| | YDVSKRPS | MGGIIPLL | | | | | | |
| | GVPDRFSGS | DSVDYAQ | | | | | | |
| | KSGNTASLT | RFQGRVT | | | | | | |
| | VSGLQAED | VSADKSTG | | | | | | |
| | EADYYCGS | TAYMEVR | | | | | | |
| | YADTDTIV | SLGSEDTA | | | | | | |
| | FGTGTKLT | KYYCAKW | | | | | | |
| | VL | SSVDTGLD | | | | | | |
| | | YWGQGTLV | | | | | | |
| | | TVSS | | | | | | |
| P2AM | N/A | QVQLQESG | N/A | N/A | N/A | NDPHY | STSHS | MRYY |
| R-12 | | PGLVKPSE | | | | WG | GHTYF | YSGTY |
| | | TLSLTCTV | | | | | NPSLKS | PVYYF |
| | | SGDSIIND | | | | | | DY |
| | | PHYWGWIR | | | | | | |
| | | QSPGKGLE | | | | | | |
| | | WIGSTSHS | | | | | | |
| | | GHTYFNPS | | | | | | |
| | | LKSRVSMS | | | | | | |
| | | IDVAKNQF | | | | | | |
| | | SLNVRSVT | | | | | | |
| | | AADTAVYY | | | | | | |
| | | CARMRYYY | | | | | | |
| | | SGTYPVYY | | | | | | |
| | | FDYWGQGT | | | | | | |
| | | LVTVSS | | | | | | |
| P2AMR-13 | SYVLTQPPS | QVQLVQSE | SGSSS | SNNQRPS | AAWDD | RYGVS | GIIPMFG | GDNSA |
| | ASGTPGQR | AEVKKPGS | NIGSN | | SLNGP | | TANYAQK | YSDAF |
| | VTISCSGSS | SVKVSCKA | FVS | | V | | FQG | NI |
| | SNIGSNFVS | SGGTFSRY | | | | | | |
| | WYQQLPGT | GVSWVRQ | | | | | | |
| | APKLLISSN | APGQGLE | | | | | | |
| | NQRPSGVP | WMGGIIP | | | | | | |
| | DRFSGSKSD | MFGTANY | | | | | | |
| | TSASLAISG | AQKFQGR | | | | | | |
| | LQSEDEAD | VTITADES | | | | | | |
| | YYCAAWD | TSTAYMEL | | | | | | |
| | DSLNGPVF | RSLRSEDT | | | | | | |
| | GGGTQLTV | AVYYCAR | | | | | | |
| | L | GDNSAYS | | | | | | |
| | | DAFNIWG | | | | | | |
| | | QGTMVTV | | | | | | |
| | | SS | | | | | | |
| OEA2-1 | DIQMTQSPS | EVQLVESG | RASQS | SASSL | QQSSS | SSSIH | SISSSS | YYGYV |
| | SLSASVGDR | GGLVQPG | VSSAV | YS | SLIT | | GSTSY | WGGY |
| | VTITCRASQ | GSLRLSCA | A | | | | ADSVK | WGWW |
| | SVSSAVAW | ASGFTFSS | | | | | G | YSKAL |
| | YQQKPGKA | SSIHWVRQ | | | | | | DY |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARYY | | | | | | |
| | | GYVWGG | | | | | | |
| | | YWGWWY | | | | | | |
| | | SKALDYW | | | | | | |
| | | GQGTLVT | | | | | | |
| | | VSS | | | | | | |
| OEA2-12 | DIQMTQSPS | EVQLVESG | RASQS | SASSL | QQYD | SSSIH | SISSYY | YQYYG |
| | SLSASVGDR | GGLVQPG | VSSAV | YS | WNYY | | GYTSY | SLYYS |
| | VTITCRASQ | GSLRLSCA | A | | LVT | | ADSVK | QQWA |
| | SVSSAVAW | ASGFTIYS | | | | | G | MDY |
| | YQQKPGKA | SSIHWVRQ | | | | | | |
| | PKLLIYSAS | APGKGLE | | | | | | |
| | SLYSGVPSR | WVASISSY | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQYDWNY | TAYLQMNS | | | | | | |
| | YLVTFGQ | LRAEDTAV | | | | | | |
| | GTKVEI | YYCARYQY | | | | | | |
| | KRTV | YGSLYYSQ | | | | | | |
| | | QWAMDYWG | | | | | | |
| | | QGTLVTVS | | | | | | |
| | | S | | | | | | |
| OEA2-16 | DIQMTQSPS | EVQLVESG | RASQS | SASSL | QQSSS | SSSIH | SISSSS | SPSSPY |
| | SLSASVGDR | GGLVQPG | VSSAV | YS | SLIT | | GSTSY | FMSW |
| | VTITCRASQ | GSLRLSCA | A | | | | ADSVK | GWYW |
| | SVSSAVAW | ASGFTFSS | | | | | G | QYGID |
| | YQQKPGKA | SSIHWVRQ | | | | | | Y |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARSPS | | | | | | |
| | | SPYFMSW | | | | | | |
| | | GWYWQY | | | | | | |
| | | GIDYWGQ | | | | | | |
| | | GTLVTVSS | | | | | | |
| OEA2-21 | DIQMTQSPS | EVQLVESG | RASQS | SASSL | QQSS | SSYIH | SISPSY | DMYE |
| | SLSASVGDR | GGLVQPG | VSSAV | YS | WGGL | | GYTSY | WWHW |
| | VTITCRASQ | GSLRLSCA | A | | VT | | ADSVK | AIDY |
| | SVSSAVAW | ASGFTFSS | | | | | G | |
| | YQQKPGKA | SYIHWVRQ | | | | | | |
| | PKLLIYSAS | APGKGLE | | | | | | |
| | SLYSGVPSR | WVASISPS | | | | | | |
| | FSGSRSGTD | YGYTSYA | | | | | | |
| | FTLTISSLQ | DSVKGRFT | | | | | | |
| | PEDFATYYC | ISADTSKN | | | | | | |
| | QQSSWGG | TAYLQMN | | | | | | |
| | LVTFGQGT | SLRAEDTA | | | | | | |
| | KVEIKRTV | VYYCARD | | | | | | |
| | | MYEWWHW | | | | | | |
| | | AIDYWGQ | | | | | | |
| | | GTLVTVSS | | | | | | |
| OEA2-24 | DIQMTQSPS | EVQLVESG | RASQS | SASSL | QQSSS | SSSIH | SISSSS | YGHYL |
| | SLSASVGDR | GGLVQPG | VSSAV | YS | SLIT | | GSTSY | YYWG |
| | VTITCRASQ | GSLRLSCA | A | | | | ADSVK | WGWY |
| | SVSSAVAW | ASGFTFSS | | | | | G | WSAAL |
| | YQQKPGKA | SSIHWVRQ | | | | | | DY |
| | PKLLIYSAS | APGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQSSSSLIT | AYLQMNS | | | | | | |
| | FGQGTKVEI | LRAEDTAV | | | | | | |
| | KRTV | YYCARYG | | | | | | |
| | | HYLYYWG | | | | | | |
| | | WGWYWSA | | | | | | |
| | | ALDYWGQ | | | | | | |
| | | GTLVTVS | | | | | | |
| | | S | | | | | | |
| BO01 | DIQMTQSPS | EVQLVESG | RASQS | SASSL | QQVS | DYSIH | SISSSS | GEWY |
| | SLSASVGDR | GGLVQPG | VSSAV | YS | YVAK | | GSTSY | AAMD |
| | VTITCRASQ | GSLRLSCA | A | | AVT | | ADSVK | Y |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQVSYVAK | AYLQMNS | | | | | | |
| | AVTFGQGT | LRAEDTAV | | | | | | |
| | KVEIKRTV | YYCARGE | | | | | | |
| | | WYAAMD | | | | | | |
| | | YWGQGTL | | | | | | |
| | | VTVSS | | | | | | |
| BO04 | DIQMTQSPS | EVQLVESG | RASQS | SASSL | QQISY | DYSIH | SISSSS | GLWSA |
| | SLSASVGDR | GGLVQPG | VSSAV | YS | VIRAL | | GSTSY | AMDY |
| | VTITCRASQ | GSLRLSCA | A | | T | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQISYVIRA | AYLQMNSL | | | | | | |
| | LTFGQGTK | RAEDTAVY | | | | | | |
| | VEIKRTV | YCARGLWS | | | | | | |
| | | AAMDYWGQ | | | | | | |
| | | GTLVTVSS | | | | | | |
| BO06 | DIQMTQSPS | EVQLVESG | RASQS | SASSL | QQTS | DYSIH | SISSSS | GPWY |
| | SLSASVGDR | GGLVQPG | VSSAV | YS | YVAR | | GSTSY | AAMD |
| | VTITCRASQ | GSLRLSCA | A | | SIT | | ADSVK | Y |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQTSYVAR | AYLQMNS | | | | | | |
| | SITFGQGTK | LRAEDTAV | | | | | | |
| | VEIKRTV | YYCARGP | | | | | | |
| | | WYAAMD | | | | | | |
| | | YWGQGTL | | | | | | |
| | | VTVSS | | | | | | |
| BO14 | DIQMTQSPS | EVQLVESG | RASQS | SASSL | QQVS | DYSIH | SISSSS | GSWA |
| | SLSASVGDR | GGLVQPG | VSSAV | YS | YVKR | | GSTSY | AAMD |
| | VTITCRASQ | GSLRLSCA | A | | EIT | | ADSVK | Y |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQVSYVKR | AYLQMNS | | | | | | |
| | EITFGQGTK | LRAEDTAV | | | | | | |
| | VEIKRTV | YYCARGS | | | | | | |
| | | WAAAMDY | | | | | | |
| | | WGQGTLV | | | | | | |
| | | TVSS | | | | | | |
| BO15 | DIQMTQSPS | EVQLVESG | RASQS | SASSL | QQVS | DYSIH | SISSSS | GLWTA |
| | SLSASVGDR | GGLVQPG | VSSAV | YS | YVIRE | | GSTSY | AMDY |
| | VTITCRASQ | GSLRLSCA | A | | LT | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQVSYVIR | AYLQMNS | | | | | | |
| | ELTFGQGT | LRAEDTAV | | | | | | |
| | KVEIKRTV | YYCARGL | | | | | | |
| | | WTAAMDY | | | | | | |
| | | WGQGTLV | | | | | | |
| | | TVSS | | | | | | |
| BO19 | DIQMTQSPS | EVQLVESG | RASQS | SASSLYS | QQISY | DYSIH | SISSSS | GHWTP |
| | SLSASVGDR | GGLVQPG | VSSAV | | VIRTV | | GSTSY | AMDY |
| | VTITCRASQ | GSLRLSCA | A | | T | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQISYVIRT | AYLQMNSL | | | | | | |
| | VTFGQGTK | RAEDTAVY | | | | | | |
| | VEIKRTV | YCARGHWT | | | | | | |
| | | PAMDYWGQ | | | | | | |
| | | GTLVTVSS | | | | | | |
| BO21 | DIQMTQSPS | EVQLVESG | RASQS | SASSL | QQISY | DYSIH | SISSSS | GPWRP |
| | SLSASVGDR | GGLVQPG | VSSAV | YS | VHRL | | GSTSY | AMDY |
| | VTITCRASQ | GSLRLSCA | A | | VT | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQISYVHR | AYLQMNS | | | | | | |
| | LVTFGQGT | LRAEDTAV | | | | | | |
| | KVEIKRTV | YYCARGP | | | | | | |
| | | WRPAMD | | | | | | |
| | | YWGQGTL | | | | | | |
| | | VTVSS | | | | | | |
| BO23 | DIQMTQSPS | EVQLVESG | RASQS | SASSLY | QQTS | DYSIH | SISSSS | GPWYP |
| | SLSASVGDR | GGLVQPG | VSSAV | | YVRR | | GSTSY | AMDY |
| | VTITCRASQ | GSLRLSCA | A | | KLT | | ADSVK | |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQISYVRR | AYLQMNS | | | | | | |
| | EITFGQGTK | LRAEDTAV | | | | | | |
| | VEIKRTV | YYCARGH | | | | | | |
| | | WHAAMDY | | | | | | |
| | | WGQGTL | | | | | | |
| | | VTVSS | | | | | | |
| BO33 | DIQMTQSPS | EVQLVESG | RASQS | SASSL | QQPS | DYSIH | SISSSS | GGWK |
| | SLSASVGDR | GGLVQPG | VSSAV | YS | YVRR | | GSTSY | PAMDY |
| | VTITCRASQ | GSLRLSCA | A | | SIT | | ADSVK | |
| | SVSSAVAW | ASGFTFSD | | | | | G | |
| | YQQKPGKAP | YSIHWVR | | | | | | |
| | KLLIYSASS | QAPGKGLE | | | | | | |
| | LYSGVPSRF | WVASISSS | | | | | | |
| | SGSRSGTDF | SGSTSYAD | | | | | | |
| | TLTISSLQP | SVKGRFTI | | | | | | |
| | EDFATYYCQ | SADTSKNT | | | | | | |
| | QPSYVRRSI | AYLQMNSL | | | | | | |
| | TFGQGTKVE | RAEDTAVY | | | | | | |
| | IKRTV | YCARGGWK | | | | | | |
| | | PAMDYWGQ | | | | | | |
| | | GTLVTVSS | | | | | | |
| BO34 | DIQMTQSPS | EVQLVESG | RASQS | SASSL | QQAS | DYSIH | SISSSS | GEWY |
| | SLSASVGDR | GGLVQPG | VSSAV | YS | YVRR | | GSTSY | AAMD |
| | VTITCRASQ | GSLRLSCA | A | | VVT | | ADSVK | Y |
| | SVSSAVAW | ASGFTFSD | | | | | G | |
| | YQQKPGKA | YSIHWVR | | | | | | |
| | PKLLIYSAS | QAPGKGLE | | | | | | |
| | SLYSGVPSR | WVASISSS | | | | | | |
| | FSGSRSGTD | SGSTSYAD | | | | | | |
| | FTLTISSLQ | SVKGRFTI | | | | | | |
| | PEDFATYYC | SADTSKNT | | | | | | |
| | QQASYVRR | AYLQMNS | | | | | | |
| | VVTFGQGT | LRAEDTAV | | | | | | |
| | KVEIKRTV | YYCARGE | | | | | | |
| | | WYAAMD | | | | | | |
| | | YWGQGTL | | | | | | |
| | | VTVSS | | | | | | |

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A binding partner that specifically binds to a peptide conjugate/MHC complex, wherein the peptide conjugate/MHC complex comprises:
(a) a peptide conjugate formed by the covalent reaction of a targeted covalent inhibitor or fragment thereof with a peptide; and
(b) an MHC.

2. The binding partner of claim 1, wherein the binding partner binds to the peptide conjugate/MHC complex with a greater affinity than to the peptide or free targeted covalent inhibitor.

3. The binding partner of claim 1 or 2, wherein the affinity of the binding partner for the peptide conjugate/MHC complex is 100-10,000 times greater than the affinity of the binding partner for the peptide or free targeted covalent inhibitor.

4. The binding partner of any one of claims 1-3, wherein the MHC is a human leukocyte antigen (HLA), optionally wherein the HLA is an HLA-A, HLA-B, or HLA-C.

5. The binding partner of claim 4, wherein the HLA molecule is an HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, and/or HLA-B*15:01 molecule.

6. The binding partner of any one of claims 1-5, wherein the peptide comprises a nucleophilic or an electrophilic residue, said residue optionally being one of cysteine, lysine, tyrosine, histidine, serine, arginine, or threonine.

7. The binding partner of any one of claims 1-6, wherein the peptide comprises a cysteine residue.

8. The binding partner of any one of claims 1-7, wherein the peptide conjugate is formed by a covalent reaction between the targeted covalent inhibitor and a cysteine residue in the peptide.

9. The binding partner of any one of claims 1-8, wherein the peptide is a segment of a protein that is associated with a cancer, optionally wherein the protein is encoded by a gene that is mutated in a cancer.

10. The binding partner of any one of claims 1-9, wherein the peptide is a segment of an enzyme, and wherein the targeted covalent inhibitor is an inhibitor of the enzyme.

11. The binding partner of claim 10, wherein the enzyme is a kinase or a GTPase.

12. The binding partner of any one of claims 1-11, wherein the peptide is or is derived from RAS, Bruton's tyrosine kinase (BTK), any epidermal growth factor receptor (EGFR) family member that is selected from EGFR (ERBB1), HER2/NEU (ERBB2), HER3 (ERBB3), and HER4 (ERBB4); MET (HGFR); any fibroblast growth factor receptor (FGFR); any cyclin-dependent kinase (CDK); Acetylcholine Esterase (ACHE); p90 ribosomal S6 kinase (RSK); TP53, IDH1, GNAS, FBXW7, CTNNB1, DNMT3A, a cathepsin that is selected from cathepsin B, C, F, H, K, L, O, S, V, W and X; any caspase; a protein involved in obesity that is optionally Pancreatic lipase or METAP2; any cancer testis antigen (CTA); a long interspersed element-1 (LINE-1); a short interspersed element that is optionally Alu; and any endogenous retroviral protein, and optionally wherein the RAS is KRAS, HRAS, or NRAS.

13. The binding partner of any one of claims 1-12, wherein the peptide comprises a segment of KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}.

14. The binding partner of any one of claims 1-13, wherein the peptide comprises the amino acid sequence of VVVGACGVGK, VVGACGVGK, or KLVVVGACGV.

15. The binding partner of any one of claims 1-14, wherein the targeted covalent inhibitor is (i) a tri-complex KRAS^{G12C} inhibitor or a KRAS^{G12C} degrader, (ii) a tri-complex KRAS^{G12D} inhibitor or a KRAS^{G12D} degrader, (iii) a tri-complex KRAS^{G12R} inhibitor or a KRAS^{G12R} degrader, or (iv) a tri-complex KRAS^{G12S} inhibitor or a KRAS^{G12S} degrader.

16. The binding partner of claim 13, wherein (i) the peptide comprises the KRAS^{G12C} mutation, and the targeted covalent inhibitor is a KRAS^{G12C} inhibitor, (ii) the peptide comprises the KRAS^{G12D} mutation, and the targeted covalent inhibitor is a KRAS^{G12D} inhibitor, (iii) the peptide comprises the KRAS^{G12R} mutation, and the targeted covalent inhibitor is a KRAS^{G12R} inhibitor, or (iv) the peptide comprises the KRAS^{G12S} mutation, and the targeted covalent inhibitor is a KRAS^{G12S} inhibitor.

17. The binding partner of any one of claims 13-16, wherein the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

18. The binding partner of any one of claims 1-12, wherein the peptide comprises a segment of EGFR.

19. The binding partner of any one of claim 1-12 or 17, wherein the peptide comprises the amino acid sequence of QLMPFGCLL, LMPFGCLLDY, or MPFGCLLDY.

20. The binding partner of any one of claims 1-12, wherein the peptide comprises a segment of EGFR and the targeted covalent inhibitor is an inhibitor of an EGFR family kinase.

21. The binding partner of any one of claims 18-20, wherein the targeted covalent inhibitor is osimertinib or neratinib.

22. The binding partner of any one of claims 1-12, wherein the peptide comprises a segment of BTK.

23. The binding partner of any one of claim 1-12 or 22, wherein the peptide comprises the amino acid sequence of YMANGCLLNY.

24. The binding partner of claim 22 or 23, wherein the targeted covalent inhibitor is ibrutinib.

25. The binding partner of any one of claims 1-24, wherein the peptide is a full length protein that is processed in the cell after the covalent reaction with the targeted covalent inhibitor, such that smaller peptide fragments are produced.

26. The binding partner of any one of claims 1-17, wherein the peptide conjugate comprises a compound selected from the group consisting of compounds 1-8: 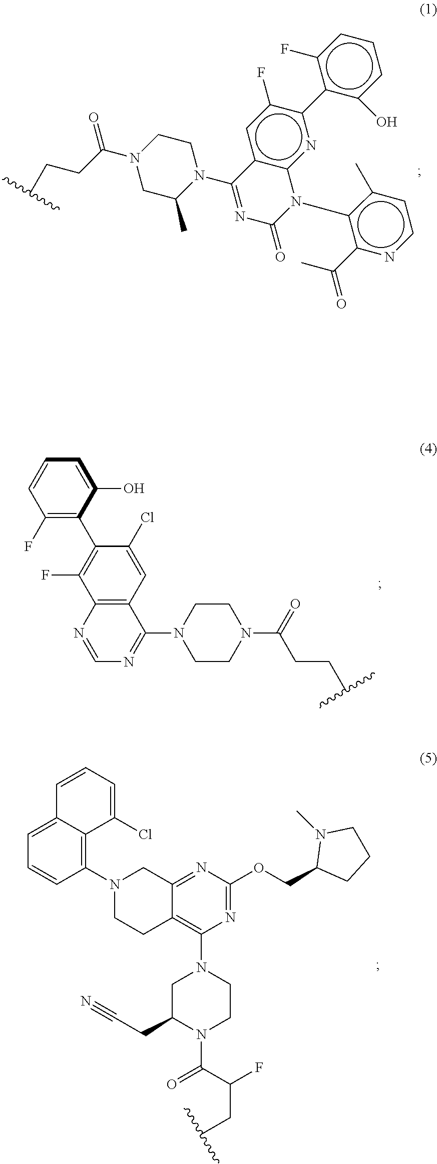 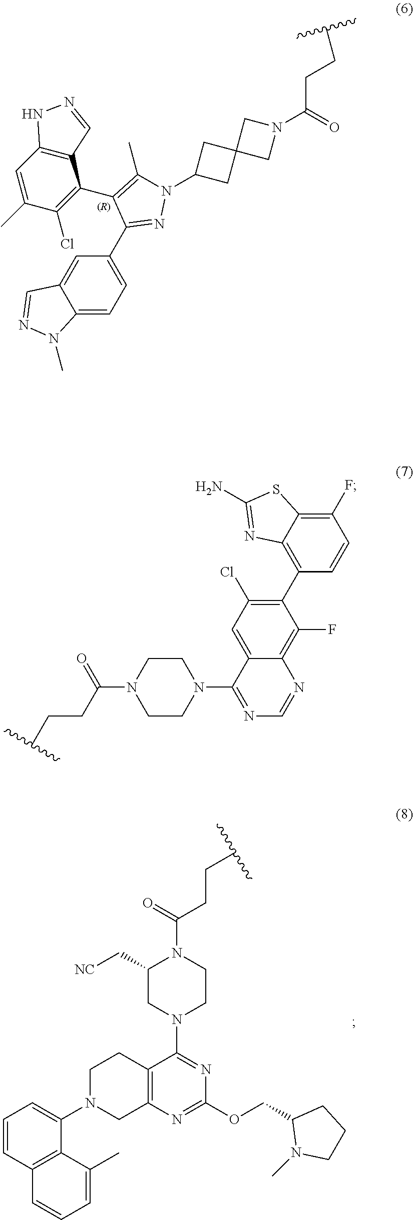
covalently bonded to the cysteine residue in a peptide comprising the amino acid sequence of VVVGACGVGK, VVGACGVGK, or KLVVVGACGV.

27. The binding partner of any one of claim 13-17, 25, or 26, wherein the MHC is HLA-A*02:01, HLA-A*03:01, and/or HLA-A*11:01.

28. The binding partner of claim 26 or 27, wherein the peptide comprises the amino acid sequence of VVVGACGVGK or VVGACGVGK and the MHC is HLA-A*03:01 or HLA-A*11:01.

29. The binding partner of claim 26 or 27, wherein the peptide comprises the amino acid sequence of KLVVVGACGV and the MHC is HLA-A*02:01.

30. The binding partner of any one of claim 1-12 or 18-21, wherein the peptide conjugate comprises compound 9: 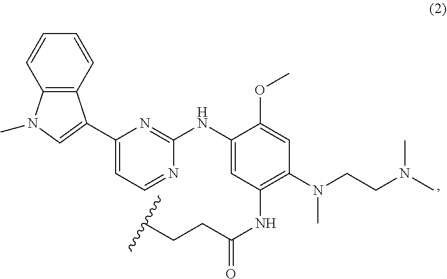
covalently bonded to the cysteine residue in a peptide comprising the amino acid sequence of QLMPFGCLL, LMPFGCLLDY, or MPFGCLLDY.

31. The binding partner of claim 30, wherein the MHC is HLA-A*02, HLA-A*01, HLA-A*03, or HLA-A*26.

32. The binding partner of any one of claim 1-12 or 22-24, wherein the peptide conjugate comprises compound 10: 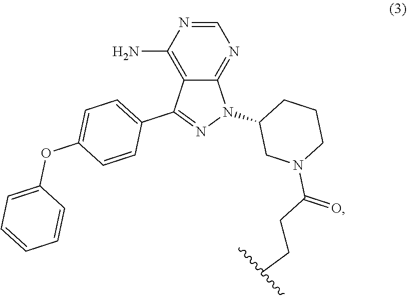 [SMILES: *CCC(=O)N1CCC[C@@H](n2nc(-c3ccc(Oc4ccccc4)cc3)c3c(N)ncnc32)C1]
covalently bonded to the cysteine residue in a peptide comprising the amino acid sequence of YMANGCLLNY.

33. The binding partner of claim 32, wherein the MHC is HLA-A*01:01.

34. The binding partner of any one of claim 1-17 or 25-29, wherein the binding partner specifically binds to a peptide conjugate/MHC complex, wherein the peptide conjugate is formed by the covalent reaction of sotorasib with a KRAS^{G12C} peptide, wherein the binding partner comprises a heavy chain variable region (VH) and a light chain variable region (VL), wherein
(a) the VH comprises:
(i) a CDR-H1 comprising the amino acid sequence of DYSIH, or a variant thereof comprising 1-3 amino acid changes;
(ii) a CDR-H2 comprising the amino acid sequence of SISSSSGSTSYADSVKG, or a variant thereof comprising 1-5 amino acid changes; and/or
(iii) a CDR-H3 comprising the amino acid sequence of GX₁WX₂X₃AMDY, wherein X₁ is G, R, H, S, or K, X₂ is Y or I, and X₃ is P or A; and/or
(b) the VL comprises:
(i) a CDR-L1 comprising the amino acid sequence of RASQSVSSAVA, or a variant thereof comprising 1-5 amino acid changes;
(ii) a CDR-L2 comprising the amino acid sequence of SASSLYS, or a variant thereof comprising 1-5 amino acid changes, and/or
(iii) a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

35. The binding partner of any one of claim 1-17 or 25-29, wherein the binding partner specifically binds to a peptide conjugate/MHC complex, wherein the peptide conjugate is formed by the covalent reaction of sotorasib with a KRAS^{G12C} peptide, wherein the binding partner comprises the CDR-H1, CDR-H2, and CDR-H3 amino acid sequences of a VH amino acid sequence and/or the CDR-L1, CDR-L2, and CDR-L3 amino acid sequences of a VL amino acid sequence of a binding partner selected from the group consisting of RA_D11, RA_D01-RA_D04, RA_D06-RA_D09, RA_D12-RA_D14, RA_D16, RA_D18-RA_D21, RA_D23, and RA_D24; or a variant thereof comprising 1-5 amino acid changes in one or more of the CDR amino acid sequences.

36. The binding partner of claim 34 or 35, wherein the binding partner comprises the CDR-H1, CDR-H2, CDR-H3, CDR-L1, CDR-L2, and/or CDR-L3 amino acid sequences of a binding partner selected from the group consisting of RA_D11, RA_D01-RA_D04, RA_D06-RA_D09, RA_D12-RA_D14, RA_D16, RA_D18-RA_D21, RA_D23, and RA_D24 (Tables G and H), or a variant thereof comprising 1-5 amino acid changes in one or more of the CDR amino acid sequences.

37. The binding partner of any one of claims 34-36, wherein the binding partner comprises a VH and/or a VL amino acid sequence that is 90%, 95%, or 100% identical to the following VH and VL sequences:
RA_D11
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRKTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWIAAMDYWGQGTLVTVSS
RA_D01
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWIAAMDYWGQGTLVTVSS
RA_D02
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQPSYVRRKITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D03
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVARKITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWIAAMDYWGQGTLVTVSS
RA_D04
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQVSYVARRITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D06
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVKRLITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGHWIAAMDYWGQGTLVTVSS
RA_D07
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRLITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D08
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRVITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWIAAMDYWGQGTLVTVSS
RA_D09
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGSWIAAMDYWGQGTLVTVSS
RA_D12
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVAKTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D13
V_{L}: DIQMTQSPSSLSASVGDRVTITCRAGQSVSSAVAWYQQKPGKAPKLLIYSASSLYSGVPS
RFSGSRSGTDFTLTISSLQPEDFATYYCQQSSYVRRKITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGKWIPAMDYWGQGTLVTVSS
RA_D14
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRKAITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGGWIAAMDYWGQGTLVTVSS
RA_D16
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRRAITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D18
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQSSYVKRTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D19
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQPSYVRKTITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D20
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVKKEITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D21
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQSSYVHKLITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGRWIPAMDYWGQGTLVTVSS
RA_D23
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVRREITFGQGTKVEIKRTV
V_{H}: EVQLVESGGGLVQPGGSLRLSCAASGFTFSDYSIHWVRQAPGKGLEWVASISSSSGSTSY
ADSVKGRFTISADTSKNTAYLQMNSLRAEDTAVYYCARGHWIAAMDYWGQGTLVTVSS
RA_D24
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQASYVHRLITFGQGTKVEIKRTV
V_{H}: EVQLVESGGGLVQPGGSLRLSCAASGFTFSDYSIHWVRQAPGKGLEWVASISSSSGSTSYADS
VKGRFTISADTSKNTAYLQMNSLRAEDTAVYYCARGRWIPAMDYWGQGTLVTVSS

38. The binding partner of any one of claim 1-12, 18-21, 30, or 31, wherein the binding partner specifically binds to a peptide conjugate/MHC complex, wherein the peptide conjugate is formed by the covalent reaction of osimertinib with an EGFR peptide, wherein the binding partner comprises a heavy chain variable region (VH) and a light chain variable region (VL), wherein
(a) the VH comprises:
(i) a CDR-H1 comprising the amino acid sequence of SSYIH, or a variant thereof comprising 1-5 amino acid changes;
(ii) a CDR-H2 comprising the amino acid sequence of YISPSYGSTSYADSVKG, or a variant thereof comprising 1-5 amino acid changes; and/or
(iii) a CDR-H3 comprising the amino acid sequence of EX₁X₂X₃MX₄X₅DY, wherein X₁ is Y, L, S, or E, X₂ is V, T, or I, X₃ is T or I, X₄ is A, T, or S, and X₅ is L, A, I, K, P, or T; and/or
(b) the VL comprises:
(i) a CDR-L1 comprising the amino acid sequence of RASQSVSSAVA, or a variant thereof comprising 1-5 amino acid changes;
(ii) a CDR-L2 comprising the amino acid sequence of SASSLYS, or a variant thereof comprising 1-5 amino acid changes; and/or
(iii) a CDR-L3 comprising the amino acid sequence of QQYX₁X₂WPX₃T, wherein X₁ is S or A, or S; X₂ is Y, H, A, D, E, K, S, or G; and X₃ is I or E.

39. The binding partner of any one of claim 1-12, 18-21, 30, or 31, wherein the binding partner specifically binds to a peptide conjugate/MHC complex, wherein the peptide conjugate is formed by the covalent reaction of osimertinib with an EGFR peptide, wherein the binding partner comprises the CDR-H1, CDR-H2, and CDR-H3 amino acid sequences of a VH amino acid sequence and/or the CDR-L1, CDR-L2, and CDR-L3 amino acid sequences of a VL amino acid sequence of a binding partner selected from the group consisting of OEA2-5, EO_Q01-EO_Q18, and EO_Q20-EO_Q24, or a variant thereof comprising 1-5 amino acid changes in one or more of the CDR amino acid sequences.

40. The binding partner of claim 38 or 39, wherein the binding partner comprises the CDR-H1, CDR-H2, CDR-H3, CDR-L1, CDR-L2, and/or CDR-L3 amino acid sequences of a binding partner selected from the group consisting of OEA2-5, EO_Q01-EO_Q18, and EO_Q20-EO_Q24 (Tables I and J), or a variant thereof comprising 1-5 amino acid changes in one or more of the CDR amino acid sequences.

41. The binding partner of any one of claims 38-40, wherein the binding partner comprises a VH and/or a VL amino acid sequence that is 90%, 95%, or 100% identical to the following VH and VL sequences:
EO_Q01
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELVTMTADYWGQGTLVTVSS
EO_Q02
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSAWPETFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYTTMSIDYWGQGTLVTVSS
EO_Q03
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSAWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARESVTMSADYWGQGTLVTVSS
EO_Q04
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARESVTMTKDYWGQGTLVTVSS
EO_Q05
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYAEWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREEVTMSIDYWGQGTLVTVSS
EO_Q06
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARESITMTKDYWGQGTLVTVSS
EO_Q07
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYAKWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELTTMSIDYWGQGTLVTVSS
EO_Q08
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELTEMTTDYWGQGTLVTVSS
EO_Q09
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREEVTMTADYWGQGTLVTVSS
EO_Q10
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSGWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELIEMTPDYWGQGTLVTVSS
EO_Q11
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSGWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELVTMTIDYWGQGTLVTVSS
EO_Q12
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYASWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELTTMSIDYWGQGTLVTVSS
EO_Q13
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELTTMTADYWGQGTLVTVSS
EO_Q14
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARESITMSPDYWGQGTLVTVSS
EO_Q15
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELIEMTTDYWGQGTLVTVSS
EO_Q16
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYVTMSIDYWGQGTLVTVSS
EO_Q17
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSSWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCARELVEMTPDYWGQGTLVTVSS
EO_Q18
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSAWPETFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYVTMALDYWGQGTLVTVSS
EO_Q20
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSDWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREEVTMTPDYWGQGTLVTVSS
EO_Q22
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPITFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYVTMALDYWGQGTLVTVSS
EO_Q23
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSHWPETFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYVTMALDYWGQGTLVTVSS
EO_Q24
V_{L}: DIQMTQSPSSLSASVGDRVTITCRASQSVSSAVAWYQQKPGKAPKLLIYSASSLYSGVPSR
FSGSRSGTDFTLTISSLQPEDFATYYCQQYSEWPETFGQGTKVEIKRTV
V_{H}: EVQLVESGGGLVQPGGSLRLSCAASGFTISSSYIHWVRQAPGKGLEWVAYISPSYGSTSY
ADSVKGRFTISADTSKNTAYLQMNSLRAEDTAVYYCAREYVTMALDYWGQGTLVTVSS

42. The binding partner of any one of the preceding claims, wherein the binding partner has a higher affinity for the peptide conjugate/MHC complex comprising a first HLA than for the peptide conjugate/MHC complex comprising a second HLA.

43. The binding partner of claim 42, wherein the first and second HLAs are each selected from the group consisting of HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, and/or HLA-B*15:01.

44. The binding partner of any one of claims 1-43, the binding partner is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BiTE), bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer.

45. The binding partner of any one of claims 1-44, wherein the binding partner is bispecific.

46. The binding partner of claim 45, wherein the binding partner specifically binds to the peptide conjugate/MHC complex and a T cell antigen.

47. The binding partner of claim 45 or 46, wherein the binding partner specifically binds to the peptide conjugate/MHC complex and human CD3.

48. The binding partner of claim 47, wherein the binding partner comprises the VH and/or VL amino acid sequences of UCHT1:
VH:
EVQLQQSGPELVKPGASMKISCKASGYSFTGYTMNWVKQSHGKNLEWMGL
INPYKGVSTYNQKFKDKATLTVDKSSSTAYMELLSLTSEDSAVYYCARSG
YYGDSDWYFDVWGQGTTLTVSS
VL:
DIQMTQTTSSLSASLGDRVTISCRASQDIRNYLNWYQQKPDGTVKLLIYY
TSRLHSGVPSKFSGSGSGTDYSLTISNLEQEDIATYFCQQGNTLPWTFAG
GTKLEIK.

49. The binding partner of any one of claims 45-48, wherein the binding partner comprises a sequence that is at least 90% similar to any of the following sequences, excluding underlined sequences:
OEA2-5_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQYSYWP
ITFGQGTKVEIKGGGGSEVQLQQSGPELVKPGASMKISCKASGYSFTGYT
MNWVKQSHGKNLEWMGLINPYKGVSTYNQKFKDKATLTVDKSSSTAYMEL
LSLTSEDSAVYYCARSGYYGDSDWYFDVWGQGTTLTVSSGGGGSGGGGSG
GGGSDIQMTQTTSSLSASLGDRVTISCRASQDIRNYLNWYQQKPDGTVKL
LIYYTSRLHSGVPSKFSGSGSGTDYSLTISNLEQEDIATYFCQQGNTLPW
TFAGGTKLEIKGGGGSEVQLVESGGGLVQPGGSLRLSCAASGFTISSSYI
HWVRQAPGKGLEWVAYISPSYGSTSYADSVKGRFTISADTSKNTAYLQMN
SLRAEDTAVYYCAREYVTMALDYWGQGTLVTVSSLEGGGGLNDIFEAQKI
EWHESRHHHHHH
EO_Q16_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQYSDWP
ITFGQGTKVEIKGGGGSEVQLQQSGPELVKPGASMKISCKASGYSFTGYT
MNWVKQSHGKNLEWMGLINPYKGVSTYNQKFKDKATLTVDKSSSTAYMEL
LSLTSEDSAVYYCARSGYYGDSDWYFDVWGQGTTLTVSSGGGGSGGGGSG
GGGSDIQMTQTTSSLSASLGDRVTISCRASQDIRNYLNWYQQKPDGTVKL
LIYYTSRLHSGVPSKFSGSGSGTDYSLTISNLEQEDIATYFCQQGNTLPW
TFAGGTKLEIKGGGGSEVQLVESGGGLVQPGGSLRLSCAASGFTISSSYI
HWVRQAPGKGLEWVAYISPSYGSTSYADSVKGRFTISADTSKNTAYLQMN
SLRAEDTAVYYCAREYVTMSIDYWGQGTLVTVSSLEGGGGLNDIFEAQKI
EWHESRHHHHHH
EO_Q17_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQYSSWP
ITFGQGTKVEIKGGGGSEVQLQQSGPELVKPGASMKISCKASGYSFTGYT
MNWVKQSHGKNLEWMGLINPYKGVSTYNQKFKDKATLTVDKSSSTAYMEL
LSLTSEDSAVYYCARSGYYGDSDWYFDVWGQGTTLTVSSGGGGSGGGGSG
GGGSDIQMTQTTSSLSASLGDRVTISCRASQDIRNYLNWYQQKPDGTVKL
LIYYTSRLHSGVPSKFSGSGSGTDYSLTISNLEQEDIATYFCQQGNTLPW
TFAGGTKLEIKGGGGSEVQLVESGGGLVQPGGSLRLSCAASGFTISSSYI
HWVRQAPGKGLEWVAYISPSYGSTSYADSVKGRFTISADTSKNTAYLQMN
SLRAEDTAVYYCARELVEMTPDYWGQGTLVTVSSLEGGGGLNDIFEAQKI
EWHESRHHHHHH

50. The binding partner of any one of claims 45-48, wherein the binding partner is a single-chain Diabody (scDb) and comprises a sequence that is at least 90% identical to any of the following sequences, excluding underlined sequences:
RA_D01_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQASYVR
RTITFGQGTKVEIKGGGGSEVQLQQSGPELVKPGASMKISCKASGYSFTG
YTMNWVKQSHGKNLEWMGLINPYKGVSTYNQKFKDKATLTVDKSSSTAYM
ELLSLTSEDSAVYYCARSGYYGDSDWYFDVWGQGTTLTVSSGGGGSGGGG
SGGGGSDIQMTQTTSSLSASLGDRVTISCRASQDIRNYLNWYQQKPDGTV
KLLIYYTSRLHSGVPSKFSGSGSGTDYSLTISNLEQEDIATYFCQQGNTL
PWTFAGGTKLEIKGGGGSEVQLVESGGGLVQPGGSLRLSCAASGFTFSDY
SIHWVRQAPGKGLEWVASISSSSGSTSYADSVKGRFTISADTSKNTAYLQ
MNSLRAEDTAVYYCARGGWIAAMDYWGQGTLVTVSSLEGGGGLNDIFEAQ
KIEWHESRHHHHHH
RA_D08_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQASYVR
RVITFGQGTKVEIKGGGGSEVQLQQSGPELVKPGASMKISCKASGYSFTG
YTMNWVKQSHGKNLEWMGLINPYKGVSTYNQKFKDKATLTVDKSSSTAYM
ELLSLTSEDSAVYYCARSGYYGDSDWYFDVWGQGTTLTVSSGGGGSGGGG
SGGGGSDIQMTQTTSSLSASLGDRVTISCRASQDIRNYLNWYQQKPDGTV
KLLIYYTSRLHSGVPSKFSGSGSGTDYSLTISNLEQEDIATYFCQQGNTL
PWTFAGGTKLEIKGGGGSEVQLVESGGGLVQPGGSLRLSCAASGFTFSDY
SIHWVRQAPGKGLEWVASISSSSGSTSYADSVKGRFTISADTSKNTAYLQ
MNSLRAEDTAVYYCARGGWIAAMDYWGQGTLVTVSSLEGGGGLNDIFEAQ
KIEWHESRHHHHHH
RA_D11_UCHT1_scDb
DIVRSDIQMTQSPSSLSASVGDRVTITCRASQSVSSAVAWYQQKPGKAPK
LLIYSASSLYSGVPSRFSGSRSGTDFTLTISSLQPEDFATYYCQQASYVR
KTITFGQGTKVEIKGGGGSEVQLQQSGPELVKPGASMKISCKASGYSFTG
YTMNWVKQSHGKNLEWMGLINPYKGVSTYNQKFKDKATLTVDKSSSTAYM
ELLSLTSEDSAVYYCARSGYYGDSDWYFDVWGQGTTLTVSSGGGGSGGGG
SGGGGSDIQMTQTTSSLSASLGDRVTISCRASQDIRNYLNWYQQKPDGTV
KLLIYYTSRLHSGVPSKFSGSGSGTDYSLTISNLEQEDIATYFCQQGNTL
PWTFAGGTKLEIKGGGGSEVQLVESGGGLVQPGGSLRLSCAASGFTFSDY
SIHWVRQAPGKGLEWVASISSSSGSTSYADSVKGRFTISADTSKNTAYLQ
MNSLRAEDTAVYYCARGGWIAAMDYWGQGTLVTVSSLEGGGGLNDIFEAQ
KIEWHESRHHHHHH.

51. The binding partner of any one of claims 1-48, wherein the binding partner comprises a heavy chain constant region selected from the group consisting of human IgM, IgG₁, IgG₂, IgG₃, IgG₄, IgA₁, and IgA₂.

52. The binding partner of claim 51, wherein the heavy chain constant region comprises one or more amino acid substitutions in the Fc region.

53. The binding partner of any one of claim 1-48, 51, or 52, wherein the binding partner comprises a human kappa light chain constant region or a human lambda light chain constant region.

54. The binding partner of any one of claims 45-50, wherein the binding partner is in a CrossMab format.

55. The binding partner of any one of claims 1-54, wherein the binding partner is conjugated to a detectable label, a chemotherapeutic agent, a radioisotope, or a toxin.

56. The binding partner of any one of claims 1-54, wherein the binding partner is comprised within a chimeric antigen receptor.

57. The binding partner of claim 56, wherein the binding partner is expressed by a T cell, macrophage, neutrophil, or natural killer cell.

58. The binding partner of any one of the preceding claims, wherein binding of the binding partner to the peptide conjugate/MHC complex is not inhibited by free targeted covalent inhibitor.

59. A complex comprising a binding partner of any one of claims 1-58 and the peptide conjugate/MHC complex.

60. A polynucleotide encoding the binding partner of any one of claims 1-58.

61. A polynucleotide encoding a heavy chain variable region and/or a light chain variable region of the binding partner of any one of claims 1-58.

62. A vector comprising the polynucleotide of claim 60 or 61.

63. The vector of claim 62, wherein the vector is a viral vector.

64. The vector of claim 63, wherein the viral vector is an adenoviral vector, lentiviral vector, retroviral vector, or adeno-associated viral vector.

65. A recombinant host cell comprising:
(a) the polynucleotide of claim 60 or 61;
(b) the vector of any one of claims 62-64;
(c) a first polynucleotide encoding a VH or a heavy chain of the binding partner of any one of claims 1-58, and a second polynucleotide encoding a VL or a light chain of the binding partner of any one of claims 1-58; or
(d) a first vector comprising a first polynucleotide encoding a VH or a heavy chain of the binding partner of any one of claims 1-58, and a second vector comprising a second polynucleotide encoding a VL or a light chain of the binding partner of any one of claims 1-58.

66. A pharmaceutical composition comprising the binding partner of any one of claims 1-58, the polynucleotide of claim 60 or 61, the vector of any one of claims 62-64, or the host cell of claim 65 and a pharmaceutically acceptable carrier or excipient.

67. A method of producing a binding partner, the method comprising culturing the host cell of claim 65 under suitable conditions so that the polynucleotide is expressed and the binding partner is produced.

68. A eukaryotic cell comprising the polynucleotide of claim 60 or 61 or the vector of any one of claims 62-64, wherein the cell is optionally a totipotent, multipotent, or pluripotent stem cell, wherein optionally the stem cell has an induced stem cell phenotype, or wherein the cell is optionally a leukocyte, optionally a CD4+ T cell, optionally a CD8+ T cell, optionally a γδ T cell, optionally a natural killer cell, a natural killer T cell, mucosal-associated invariant T (MAIT) cell, a neutrophil, or a macrophage.

69. A method comprising administering to an individual in need thereof the binding partner of any one of claims 1-58, the polynucleotide of claim 60 or 61, the vector of any one of claims 62-64, the pharmaceutical composition of claim 66, or the cell of claim 68.

70. A method for generating a peptide conjugate/MHC complex, the method comprising contacting a cell with a targeted covalent inhibitor, and isolating the peptide conjugate/MHC complex.

71. The method of claim 70, further comprising identifying the peptide conjugate/MHC complex.

72. A cell free peptide conjugate/MHC complex comprising:
(a) an isolated peptide conjugate formed by the covalent reaction of a targeted covalent inhibitor with a peptide; and
(b) an MHC.

73. The peptide conjugate/MHC complex of claim 72, wherein RAS, EGFR, BTK, HER2/NEU (ERBB2), HER3 (ERBB3), HER4 (ERBB4), MET (HGFR); FGFR, CDK, Acetylcholine Esterase (ACHE), p90 ribosomal S6 kinase (RSK), TP53, IDH1, GNAS, FBXW7, CTNNB1, DNMT3A, cathepsin B, cathepsin C, cathepsin F, cathepsin H, cathepsin K, cathepsin L, cathepsin O, cathepsin S, cathepsin V, cathepsin W, cathepsin X, a caspase, pancreatic lipase, METAP2, any cancer testis antigen (CTA), a long interspersed element-1 (LINE-1), a short interspersed element that is optionally Alu, or any endogenous retroviral protein, and optionally wherein the RAS is a KRAS, a HRAS or an NRAS.

74. The antigen of claim 72 or 73, wherein the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

75. A cell free peptide conjugate/MHC complex comprising
(a) a compound selected from the group consisting of compounds 1 and 4-8, 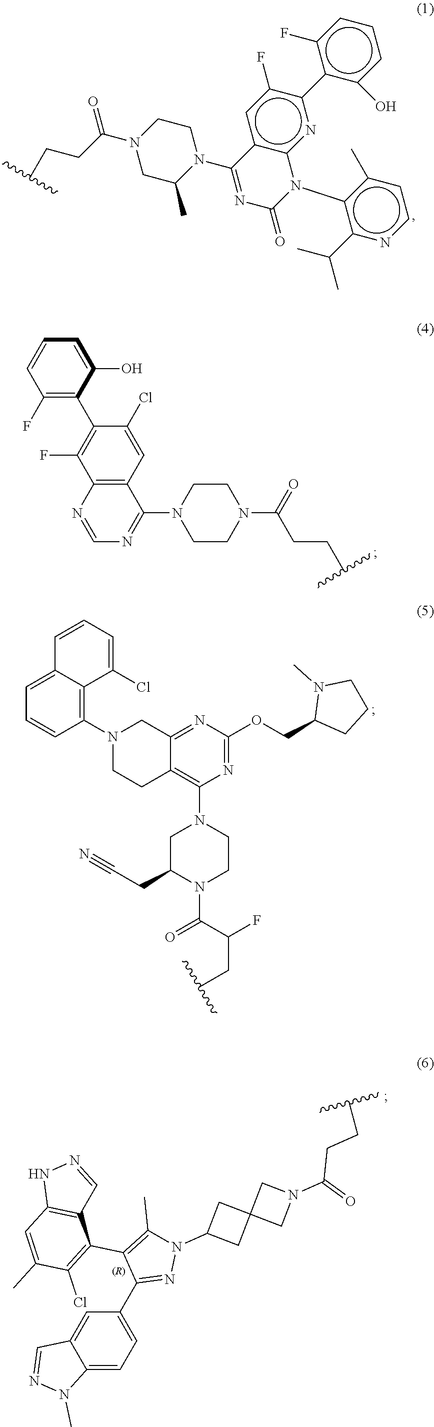 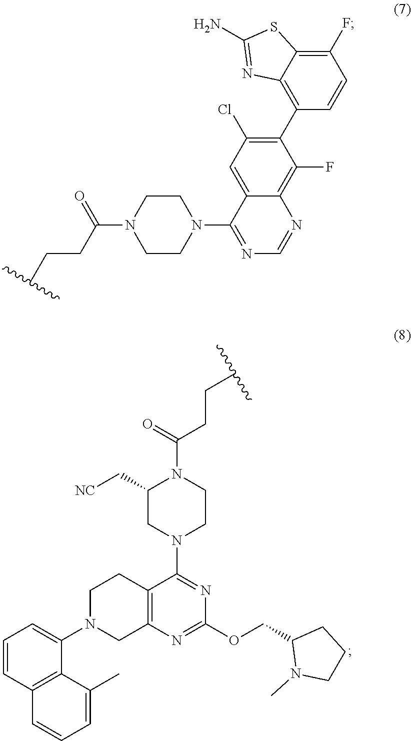
covalently bonded to a cysteine residue in a peptide comprising the amino acid sequence of VVVGACGVGK, VVGACGVGK, or KLVVVGACGV; and
(b) an MHC.

76. A cell-free peptide conjugate/MHC complex comprising
(a) compound 2: 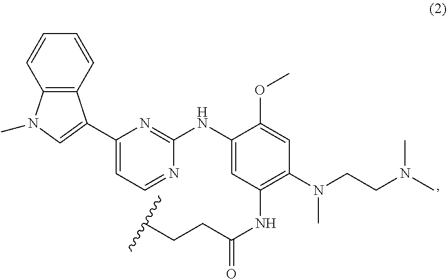
covalently bonded to the cysteine residue in a peptide comprising the amino acid sequence of QLMPFGCLL, LMPFGCLLDY, or MFPGCLLDY; and
(b) an MHC.

77. A cell-free peptide conjugate/MHC complex comprising
(a) compound 3: 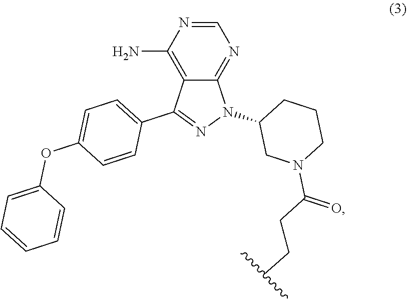 [SMILES: *CCC(=O)N1CCC[C@@H](n2nc(-c3ccc(Oc4ccccc4)cc3)c3c(N)ncnc32)C1]
covalently bonded to the cysteine residue in a peptide comprising the amino acid sequence of YMANGCLLNY; and
(b) an MHC.

78. The peptide conjugate/MHC complex of any one of claims 72-77, wherein the MHC is an HLA, optionally wherein the HLA is HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, or HLA-B*15:01.

79. A recombinant cell or particle comprising on its outer surface the cell free peptide conjugate/MHC complex of any one of claims 72-78.

80. A method of identifying one or more binding partners that bind with specificity to a peptide conjugate presented by an HLA, the method comprising contacting the cell-free peptide conjugate/MHC complex of any one of claims 72-78 with a plurality of binding partners, and selecting one or more binding partners that bind with specificity to the cell-free peptide conjugate/MHC complex.

81. The method of claim 80, further comprising determining the sequence of the one or more selected binding partners.

82. The method of claim 80 or 81, further comprising producing the one or more selected binding partners.

83. A method for identifying a binding partner that specifically binds to a peptide conjugate presented by two or more HLAs, the method comprising providing a sample of cells from a subject who has been treated with a targeted covalent inhibitor and has different HLA types, or providing the cell free peptide conjugate/MHC complex of any one of claims 72-78 presented by two or more HLAs, and screening binding partners to thereby identify a binding partner that binds with specificity to the peptide conjugate or the antigen that is presented by more than one HLA.

84. The method of claim 74, wherein the two or more HLAs types comprise HLA-A*02:01 and at least one additional HLA type.

85. The method of claim 74 or 75, wherein the two or more HLAs are each HLA-A*02:01, HLA-A*03:01, HLA-A*01:01, HLA-A*11:01, HLA-A*24:02, HLA-A*26:01, HLA-B*07:02, HLA-B*08:01, HLA-B*27:05, HLA-B*39:01, HLA-B*40:01, HLA-B*58:01, or HLA-B*15:01.

86. A method of killing a cancer cell in a subject, the method comprising administering to the subject:
(a) a targeted covalent inhibitor, and
(b) the binding partner of any one of claims 1-58, the polynucleotide of claim 60 or 61, the vector of any one of claims 62-64, the pharmaceutical composition of claim 66, or the cell of claim 68.

87. The method of claim 86, wherein the targeted covalent inhibitor targets RAS, Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, any cancer testis antigen, an endogenous retroviral protein, a long interspersed element-1 (LINE-1), or a short interspersed element (SINE), and optionally wherein the RAS is a KRAS, a HRAS or an NRAS.

88. A method of targeting a cell that expresses EGFR, BTK, or RAS mutation in a subject that has been treated with an EGFR, BTK, or RAS targeted covalent inhibitor, the method comprising administering to the subject a binding partner of any one of claims 1-58, the polynucleotide of claim 60 or 61, the vector of any one of claims 62-64, the pharmaceutical composition of claim 66, or the cell of claim 68.

89. The method of claim 88, wherein the subject has cancer.

90. A method of treating cancer in a subject that has been treated with a targeted covalent inhibitor, the method comprising administering to the subject a binding partner of any one of claims 1-58, the polynucleotide of claim 60 or 61, the vector of any one of claims 62-64, the pharmaceutical composition of claim 66, or the cell of claim 68.

91. A method of treating a disease or disorder in a subject that has been treated with a targeted covalent inhibitor, the method comprising administering to the subject a binding partner of any one of claims 1-58, the polynucleotide of claim 60 or 61, the vector of any one of claims 62-64, the pharmaceutical composition of claim 66, or the cell of claim 68.

92. The method of claim 91, wherein the disease or disorder is an autoimmune disease or a fibrotic disease.

93. A method of enhancing immune recognition of a cell expressing a RAS or EGFR mutation in a subject that has a cancer that exhibits a RAS or EGFR mutation, the method comprising administering to the subject:
(a) a RAS or EGFR inhibitor, and
(b) the binding partner of any one of claims 1-58, the polynucleotide of claim 60 or 61, the vector of any one of claims 62-64, the pharmaceutical composition of claim 66, or the cell of claim 68, and optionally wherein the RAS mutation is KRAS^{G12C}, KRAS^{G12D}, KRAS^{G12R}, KRAS^{G12S}, HRAS^{G12C}, HRAS^{G12D}, HRAS^{G12R}, HRAS^{G12S} NRAS^{G12C}, NRAS^{G12D}, NRAS^{G12R}, or NRAS^{G12S}.

94. The method of claim 93, wherein the subject has been previously treated with the RAS or EGFR inhibitor.

95. The method of claim 93 or 94, wherein the inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

96. The method of any one of claims 90-92, wherein the targeted covalent inhibitor targets RAS, Bruton's tyrosine kinase (BTK), EGFR (ERBB1), HER2/NEU (ERBB2), HER3 (ERBB3), HER4 (ERBB4), a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, Pancreatic lipase, METAP2, any cancer testis antigen, an endogenous retroviral protein, a long interspersed element-1 (LINE-1), or a short interspersed element (SINE).

97. The method of any one of claim 86, 87, 89, 90, or 93-96, wherein the cancer is renal cell carcinoma, breast cancer, prostate cancer, pancreatic cancer, lung cancer, liver cancer, ovarian cancer, cervical cancer, colorectal cancer, esophageal cancer, glioma, glioblastoma, brain cancer, stomach cancer, bladder cancer, testicular cancer, head and neck cancer, melanoma, skin cancer, sarcoma, fibrosarcoma, angiosarcoma, osteosarcoma, rhabdomyosarcoma, leukemia, lymphoma, or myeloma.

98. The method of any one of claims 86-90, wherein the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

99. The method of any one of claims 86-98, further comprising administering an additional therapeutic agent.

100. The method of claim 99, wherein the additional therapeutic agent is a chemotherapy, an immunomodulator, or another targeted covalent inhibitor.

101. The method of claim 100, wherein the immunomodulator is a checkpoint targeting agent, optionally wherein the checkpoint targeting agent is selected from the group consisting of an antagonist anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, and an agonist anti-CD28 antibody; a cytokine, optionally wherein the cytokine is an anchored IL2 or an engineered IL2; or an inhibitor of extracellular adenosine (eADO) signaling.

102. A method of detecting a peptide conjugate/MHC complex in a biological sample, the method comprising contacting the sample with the binding partner of any one of claims 1-58, wherein the peptide conjugate/MHC complex comprises a peptide conjugate formed by the covalent reaction of a targeted covalent inhibitor with a peptide.

103. The method of claim 102, wherein the peptide is an EGFR, BTK, or RAS peptide, and optionally wherein the RAS peptide is a KRAS^{G12C} peptide, a KRAS^{G12D} peptide, a KRAS^{G12R} peptide, a KRAS^{G12S} peptide, a HRAS^{G12C} peptide, a HRAS^{G12D} peptide, a HRAS^{G12R} peptide, a HRAS^{G12S} peptide, a NRAS^{G12C} peptide, a NRAS^{G12D} peptide, a NRAS^{G12R} peptide, or a NRAS^{G12S} peptide.

104. The method of claim 102 or 103, wherein the targeted covalent inhibitor is an EGFR, BTK, or KRAS^{G12C} inhibitor.

105. The method of any one of claims 102-104, wherein the targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

106. The method of any one of claims 102-105, wherein the biological sample is blood or serum.

107. A method of identifying a cell containing the peptide conjugate/MHC complex of any one of claims 72-78, the method comprising contacting the cell with the binding partner of any one of claims 1-58.

108. A method for generating one or more binding partners that specifically bind to a peptide conjugate, wherein said peptide conjugate comprises a peptide covalently bound to a non-peptide molecule, the method comprising:
a) exposing said peptide conjugate to a plurality of binding partners, and
b) selecting binding partners that specifically bind to said peptide conjugate to provide one or more selected binding partners.

109. The method of claim 108, wherein step (b) comprises selecting binding partners that specifically bind to said peptide conjugate but do not detectably bind, or bind with lower affinity, to said peptide or to said non-peptide molecule when they are not covalently bound to each other.

110. A method for generating one or more binding partners that specifically bind to a peptide conjugate presented in the context of a MHC molecule or a fragment or derivative thereof, wherein said peptide conjugate comprises a peptide covalently bound to a non-peptide molecule, the method comprising:
a) providing a complex of said peptide conjugate with said MHC molecule or the fragment or derivative thereof;
b) exposing said complex to a plurality of binding partners, and
c) selecting binding partners that specifically bind to said complex to provide one or more selected binding partners.

111. The method of claim 110, wherein step (c) comprises selecting binding partners that specifically bind to said complex but do not detectably bind or bind with lower affinity to a complex of said peptide with said MHC molecule or fragment thereof, wherein said peptide is not covalently bound to said non-peptide molecule.

112. The method of claim 110 or claim 111, wherein step (c) comprises selecting binding partners that specifically bind to said peptide conjugate presented in the context of two or more different MHC molecules.

113. The method of any one of claims 110-112, wherein the MHC molecule is MHC class I molecule and the peptide is 7-15 amino acids long, or wherein the MHC molecule is a non-classical MHC class I molecule and the peptide is 7-15 amino acids long.

114. The method of any one of claims 110-112, wherein the MHC molecule is MHC class II molecule and the peptide is 9-30 amino acids long.

115. The method of any one of claims 110-114, wherein said complex is immobilized on a solid support.

116. The method of any one of claims 110-114, wherein said complex is present on a surface of a cell.

117. The method of claim 116, further comprising determining whether said one or more binding partners selected in step (c) can mediate immune cell-mediated killing or antibody-dependent cellular cytotoxicity (ADCC), antibody dependent cellular phagocytosis (ADCP), or complement dependent cytotoxicity (CDC) mediated killing of said cell, or by use of said binding partners as immunotoxins or as antibody-drug conjugates (ADCs) or as radioconjugates.

118. The method of any one of claims 110-117, wherein said plurality of binding partners is generated by a phage-display library or yeast-display library.

119. A method of killing a cancer cell, the method comprising administering to said cell a binding partner that specifically binds to a peptide conjugate, wherein said peptide conjugate comprises (i) a peptide derived from a target protein within said cell which peptide is covalently bound to (ii) a non-peptide molecule, and wherein said binding partner mediates an immune cell-mediated killing or antibody-drug conjugate (ADC)-mediated, antibody dependent cellular phagocytosis (ADCP), or complement dependent cytotoxicity (CDC) killing of said cell, or by using the binding partner as an immunotoxin, or by action of a radioconjugate, to thereby kill the cancer cell.

120. The method of claim 119, wherein said peptide conjugate is presented on said cell in the context of an MHC molecule.

121. The method of claim 120, wherein said binding partner specifically binds to a complex comprising said peptide conjugate and said MHC molecule.

122. The method of claim 121, wherein said binding partner does not detectably bind to a complex of said peptide with said MHC molecule, wherein said peptide is not covalently bound to said non-peptide molecule.

123. The method of any one of claims 119-122, further comprising administering to said cell said non-peptide molecule, wherein said non-peptide molecule forms a covalent bond with said target protein in said cell.

124. The method of claim 123, wherein said non-peptide molecule is administered to said cell prior to administering said binding partner.

125. The method of any one of claims 119-124, wherein said cell is isolated from a subject.

126. The method of any one of claims 119-125, wherein said cell is in a subject and said binding partner is administered to the subject.

127. The method of claim 123 or claim 124, wherein said cell is in a subject and said non-peptide molecule and said binding partner are administered to the subject.

128. The method of any one of claims 119-127, wherein said target protein wherein said target protein is alternatively spliced or over-expressed in cancer cells but is not alternatively spliced or over-expressed by cancer cells.

129. The method of claim 128, wherein said target protein is encoded by a gene that is mutated in cancer cells but not mutated in non-cancer cells.

130. The method of claim 128 or claim 129, wherein said non-peptide molecule is a covalent inhibitor of said target protein.

131. The method of any one of claims 119-131, wherein said binding partner is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a Diabody (Db), a Dual-Affinity Retargeting (DART) molecule, a single-chain variable fragment (scFv), a bispecific T-cell engager (BiTE), a bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer or comprises a radioconjugate.

132. The method of claim 131, wherein said CAR is present on a T cell, natural killer (NK) cell, neutrophil, or macrophage.

133. The method of any one of claims 119-130, wherein said binding partner is an antibody-drug conjugate (ADC), a radioconjugate, or toxin conjugate.

134. The method of any one of claims 119-133, wherein said target protein is selected from KRAS, Bruton's tyrosine kinase (BTK), a member of epidermal growth factor receptor (EGFR) family, a fibroblast growth factor receptor (FGFR), MET, BRAF, a cyclin-dependent kinase (CDK), Acetyl Choline Esterase (ACHE), TP53, IDH1, GNAS, FBXW7, CTNNB1, DNMT3A, a cathepsin, a caspase, pancreatic lipase, METAP2, a Cancer Testis Antigen, viral polymerase, a protein required for viral cell entry, a protein encoded by a transposable element (e.g. an endogenous retrovirus), or a mutant thereof.

135. The method of claim 134, wherein said target protein is a KRAS protein comprising G12C mutation and said non-peptide molecule is selected from sotorasib, ARS-853, ARS-1620, ARS-3248, MRTX849, JNJ74699157, LY3499446, LY3537982, MRTX-1257, JDQ443, RMC-6291, GDC-6036, D-1553, 2E07, 6H05, SML-8-73-1, or BI 182391, and derivatives thereof.

136. The method of claim 134, wherein said target protein is EGFR and said non-peptide molecule is selected from PD168393, PF00299804 (dacomitinib), EKB569 (pelitinib), afatinib, WZ4002, osimertinib (AZD9291), PF-06459988, nazartinib, naquotinib, olmutinib, avitinib, rociletinib, neratinib, pyrotinib, poziotinib, and derivatives thereof.

137. The method of claim 134, wherein said target protein is Bruton's tyrosine kinase (BTK) and said non-peptide molecule is selected from ibrutinib, acalabrutinib, zanubrutinib, CHMFL-BTK-11, ONO/GS-405, PRN1008, CC-292, and derivatives thereof.

138. The method of claim 134, wherein said target protein is p90 ribosomal S6 kinase (RSK) and said non-peptide molecule is fluoromethylketone (FMK), dimethyl fumarate, or derivatives thereof.

139. The method of claim 134, wherein said target protein is FGFR and said non-peptide molecule is selected from FIIN-1, FIIN-2, FIIN-3, BGJ398, AZD4547, PRN1371, FGF401, and derivatives thereof.

140. A method of treating a cancer in a subject in need thereof, the method comprising administering to said subject an effective amount of a binding partner that specifically binds to a peptide conjugate, wherein said peptide conjugate comprises (i) a peptide derived from a target protein present in cancer cells of said subject, which peptide is covalently bound to (ii) a non-peptide molecule, and wherein said binding partner mediates immune cell-mediated killing or antibody-drug conjugates (ADC)-mediated killing of cancer cells, antibody dependent cellular phagocytosis (ADCP), or complement dependent cytotoxicity (CDC) mediated killing of said cancer cell, or by use of said binding partners as immunotoxins or radioconjugates in said subject, to thereby kill the cancer cell.

141. The method of claim 140, wherein said peptide conjugate is presented on cancer cells of said subject in the context of a MHC molecule.

142. The method of claim 140, wherein said binding partner specifically binds to a complex comprising said peptide conjugate and said MHC molecule.

143. The method of claim 142, wherein said binding partner does not detectably bind to a complex of said peptide with said MHC molecule, wherein said peptide is not covalently bound to said non-peptide molecule.

144. The method of any one of claims 140-143, wherein said subject has previously received said non-peptide molecule.

145. The method of any one of claims 140-143, further comprising administering to said subject an effective amount of said non-peptide molecule, wherein said non-peptide molecule forms a covalent bond with said target protein in cancer cells of said subject.

146. The method of claim 145, wherein said non-peptide molecule is administered to said subject prior to administering said binding partner.

147. A method for improving efficacy of an anti-cancer treatment in a subject in need thereof, wherein said anti-cancer treatment comprises administering to said subject a non-peptide molecule, which non-peptide molecule forms a covalent bond with a target protein in cancer cells of said subject, the method comprising further administering to said subject an effective amount of a binding partner that specifically binds to a peptide conjugate, wherein said peptide conjugate comprises a peptide derived from said target protein covalently bound to said non-peptide molecule, and wherein said binding partner mediates immune cell-mediated killing or antibody-drug conjugate (ADC)-mediated killing of cancer cells, antibody dependent cellular phagocytosis (ADCP), or complement dependent cytotoxicity (CDC) mediated killing of said cancer cell, or by use of said binding partners as immunotoxins, or by use of said binding partners as radioconjugates, in said subject.

148. The method of claim 147, wherein said peptide conjugate is presented on cancer cells of said subject in the context of an MHC molecule.

149. The method of claim 148, wherein said binding partner specifically binds to a complex comprising said peptide conjugate and said MHC molecule.

150. The method of claim 149, wherein said binding partner does not detectably bind to a complex of said peptide with said MHC molecule, wherein said peptide is not covalently bound to said non-peptide molecule.

151. The method of any one of claims 147-149, wherein said subject has previously received said non-peptide molecule prior to administering said binding partner.

152. A kit comprising (i) a non-peptide molecule, wherein said non-peptide molecule forms a covalent bond with a target protein in a cell, (ii) a binding partner that specifically binds to a peptide conjugate, wherein said peptide conjugate comprises a peptide derived from said target protein covalently bound to said non-peptide molecule, and optionally (iii) instructions for use.

153. An isolated peptide conjugate comprising a peptide of 7-30 amino acids in length comprising an amino acid sequence that is at least 80% identical to the amino acid sequence VVGACGVGK, wherein the peptide is conjugated to sotorasib or a derivative thereof.

154. An isolated peptide conjugate comprising a peptide of 7-30 amino acids in length comprising an amino acid sequence that is at least 80% identical to the amino acid sequence QLMPFGCLL, wherein the peptide is conjugated to osimertinib or a derivative thereof.

155. An isolated peptide conjugate comprising a peptide of 7-30 amino acids in length comprising an amino acid sequence that is at least 80% identical to the amino acid sequence YMANGCLLNY, wherein the peptide is conjugated to ibrutinib or a derivative thereof.

156. An isolated molecular complex comprising the peptide conjugate of any one of claims 46-48 and a MHC molecule, or a fragment or derivative thereof.

157. A host cell comprising the molecular complex of claim 156.

158. A solid surface carrier comprising the molecular complex of claim 156.

159. A kit comprising (i) the peptide conjugate of any one of claims 153-155, the molecular complex of claim 156, the cell of claim 157, the solid surface carrier of claim 158, or any combination thereof, and optionally (ii) instructions for use.

160. An isolated binding partner that specifically binds the peptide conjugate of any one of claims 46-48 or the molecular complex of claim 156.

161. The isolated binding partner of claim 160, wherein said binding partner is an antibody or an antigen-binding fragment thereof.

162. The isolated binding partner of claim 161, wherein said binding partner is a component of a chimeric antigen receptor (CAR).

163. A fusion protein comprising the binding partner of any of the preceding claims, said fusion protein comprising (i) at least one cytokine, and/or (ii) an additional antibody or fragment thereof that is an immune checkpoint inhibitor.

164. The binding partner of claim 163, wherein (i) the cytokine is selected from the group consisting of interleukin (IL)-2, IL-7, IL-8, IL-15, IL-17, and a combination thereof, and/or (ii) the additional antibody or fragment thereof that is an immune checkpoint inhibitor is an anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.

165. The binding partner of any one of the preceding claims, wherein the binding partner comprises a component that binds to a T cell or natural killer (NK) cell protein, wherein said T cell or NK cell protein is selected from the group consisting of a T cell receptor protein, CD4, CD8, CD28, CD16A, NKG2D, NKp30, NKp46, and a combination thereof.

166. A polypeptide comprising an antigen-binding domain comprising a heavy chain variable region (VH) and/or a light chain variable region (VL), wherein
(a) the VH comprises:
i. a heavy chain complementarity determining region 3 (CDR-H3) comprising the amino acid sequence of GX₁WX₂X₃AMDY, wherein X₁ is G, R, H, S, or K, X₂ is Y or I, and X₃ is P or A; and/or
(b) the VL comprises:
i. a light chain complementarity determining region 3 CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

167. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide binds to an epitope of the MHC.

168. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the antigen-binding domain binds to the peptide conjugate/MHC complex with a dissociation constant (K_{D}) of at most about 50 nM.

169. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the antigen-binding domain binds to the free targeted covalent inhibitor with a dissociation constant (K_{D}) of at least 200 nM.

170. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein:
(a) the polypeptide binds to the peptide conjugate/MHC complex at an angle from about 10° to 60° between the axis of the MHC and the axis of the polypeptide;
(b) when the polypeptide is bound to the peptide conjugate/MHC complex, the angle between the axis of the polypeptide and the axis of the peptide conjugate/MHC complex is from about 10° to 60°;
(c) when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex;
(d) when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex;
(e) the polypeptide binds to the peptide conjugate/MHC complex at an angle that is different than the angle to which a T cell receptor binds to the peptide conjugate/MHC complex; and/or
(f) the polypeptide binds to the peptide conjugate/MHC complex at an angle that is from about 10° to 60° less or from about 10° to 60° more than the angle to which a T cell receptor binds to the peptide conjugate/MHC complex.

171. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide contacts one or more residues of an alpha 1 domain or region and one or more residues of an alpha 2 domain or region of a heavy chain of the MHC of the peptide conjugate/MHC complex.

172. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein the polypeptide binds to residues 62-66, 106-109, and/or 150-170 of the MHC, or one or more residues thereof.

173. A polypeptide comprising an antigen binding domain, wherein the antigen binding domain binds to a peptide conjugate/MHC complex, wherein the peptide conjugate of the peptide conjugate/MHC complex is a peptide covalently linked to a targeted covalent inhibitor or fragment thereof; and wherein an interface area of the polypeptide with the peptide conjugate/MHC complex is at least about 500 Å².

174. The polypeptide of any one of claims 166-173, wherein
(a) the VH comprises a CDR-H3 comprising the amino acid sequence of GX₁WX₂X₃AMDY, wherein X₁ is G, R, H, S, or K, X₂ is Y or I, and X₃ is P or A; and/or
(b) the VL comprises a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

175. The polypeptide of any one of claims 166-174, wherein the VH comprises:
(i) a CDR-H1 comprising the amino acid sequence of DYSIH, or a variant thereof comprising 1-3 amino acid changes;
(ii) a CDR-H2 comprising the amino acid sequence of SISSSSGSTSYADSVKG, or a variant thereof comprising 1-5 amino acid changes; and/or
(iii) a CDR-H3 comprising the amino acid sequence of GX₁WX₂X₃AMDY, wherein X₁ is G, R, H, S, or K, X₂ is Y or I, and X₃ is P or A.

176. The polypeptide of any one of claims 166-175, wherein the VL comprises:
(i) a CDR-L1 comprising the amino acid sequence of RASQSVSSAVA, or a variant thereof comprising 1-5 amino acid changes;
(ii) a CDR-L2 comprising the amino acid sequence of SASSLYS, or a variant thereof comprising 1-5 amino acid changes, and/or
(iii) a CDR-L3 comprising the amino acid sequence of QQX₁SYVX₂X₃X₄IT, wherein X₁ is I, A, P, V, or S, X₂ is K, R, A, or H, X₃ is K or R, and X₄ is L, T, K, R, V, A, or E.

177. The polypeptide of claim 166, wherein the VH comprises a CDR-H1 having a sequence of DYSIH, a CDR-H2 having a sequence of SISSSSGSTSYADSVKG, and a CDR-H3 having a sequence of GGWIAAMDY.

178. The polypeptide of claim 166 or 177, wherein the VL comprises a CDR-L1 having a sequence of RASQSVSSAVA, a CDR-L2 having a sequence of SASSLYS, and a CDR-L3 having a sequence of QQASYVRKTIT.

179. The polypeptide of any one of claims 166-178, wherein the VH comprises an amino acid sequence having at least about 90%, 95%, or 100% sequence identity to the amino acid sequence
EVQLVESGGGLVQPGGSLRLSCAASGFTFSDYSIHWVRQAPGKGLEWVAS
ISSSSGSTSYADSVKGRFTISADTSKNTAYLQMNSLRAEDTAVYYCARGG
WIAAMDYWGQGTLVTVSS.

180. The polypeptide of any one of claims 166-179, wherein the VL comprises an amino acid sequence having at least about 90%, 95%, or 100% sequence identity to the amino acid sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

181. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GSWIHAMDY.

182. The polypeptide of claim 181, wherein the VH comprises a CDR-H2 comprising the amino acid sequence of SISSSWGVTSYADSVKG.

183. The polypeptide of claim 181 or 182, wherein the VH comprises a CDR-H1 comprising the amino acid sequence of FHWYSIH.

184. The polypeptide of any one of claims 181-183, wherein the antigen-binding domain further comprises a light chain complementarity determining region 3 (VL), and wherein the VL comprises a CDR-L3 comprising the amino acid sequence of QQASYVRKTIT.

185. The polypeptide of claim 184, wherein the VL comprises a CDR-L2 comprising the amino acid sequence of SASSLYS.

186. The polypeptide of claim 184 or 185, wherein the VL comprises a CDR-L1 comprising the amino acid sequence of RASQSVSSAVA.

187. The polypeptide of any one of claims 181-186, wherein the antigen binding domain comprises:
a CDR-H3 sequence of GSWIHAMDY,
a CDR-H2 sequence of SISSSWGVTSYADSVKG,
a CDR-H1 sequence of FHWYSIH,
a CDR-L3 sequence of QQASYVRKTIT,
a CDR-L2 sequence of SASSLYS, and
a CDR-L1 sequence of RASQSVSSAVA.

188. The polypeptide of any one of claims 181-187, wherein the VH comprises a sequence with at least 80% sequence identity to the sequence
EVQLVESGGGLVQPGGSLRLSCAASGFTFHWYSIHWVRQAPGKGLEWVAS
ISSSWGVTSYADSVKGRFTISADTSKNTAYLQMNSLRAEDTAVYYCARGS
WIHAMDYWGQGTLVTVSS.

189. The polypeptide of any one of claims 184-187, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

190. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GHWIAAMDY.

191. The polypeptide of claim 190, wherein the VH comprises a CDR-H2 comprising the amino acid sequence of SIASSSGSTGYADSVKG.

192. The polypeptide of claim 190 or 191, wherein the VH comprises a CDR-H1 comprising the amino acid sequence of FSWYSIH.

193. The polypeptide of any one of claims 190-192, wherein the antigen-binding domain further comprises a light chain variable region (VL), and wherein the VL comprises a light chain complementarity determining region 3 (CDR-L3) comprising the amino acid sequence of QQASYVRKTIT.

194. The polypeptide of claim 193, wherein the VL comprises a CDR-L2 comprising the amino acid sequence of SASSLYS.

195. The polypeptide of claim 193 or 194, wherein the VL comprises a CDR-L1 comprising the amino acid sequence of RASQSVSSAVA.

196. The polypeptide of any one of claims 190-195, wherein the antigen binding domain comprises:
a CDR-H3 sequence of GHWIAAMDY,
a CDR-H2 sequence of SIASSSGSTGYADSVKG,
a CDR-H1 sequence of FSWYSIH,
a CDR-L3 sequence of QQASYVRKTIT,
a CDR-L2 sequence of SASSLYS, and
a CDR-L1 sequence of RASQSVSSAVA.

197. The polypeptide of any one of claims 190-196, wherein the VH comprises a sequence with at least 80% sequence identity to the sequence
EVQLVESGGGLVQPGGSLRLSCAASGFTFSWYSIHWVRQAPGKGLEWVAS
IASSSGSTGYADSVKGRFTISADTSKNTAYLQMNSLRAEDTAVYYCARGH
WIAAMDYWGQGTLVTVSS.

198. The polypeptide of any one of claims 193-196, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

199. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GGVIHAMDY.

200. The polypeptide of claim 199, wherein the VH comprises a CDR-H2 comprising the amino acid sequence of SILSRWGVTSYADSVKG.

201. The polypeptide of claim 199 or 200, wherein the VH comprises a CDR-H1 comprising the amino acid sequence of FSPYSIH.

202. The polypeptide of any one of claims 199-201, wherein the antigen-binding domain further comprises a light chain complementarity determining region 3 (VL), and wherein the VL comprises a CDR-L3 comprising the amino acid sequence of QQASYVRKTIT.

203. The polypeptide of claim 202, wherein the VL comprises a CDR-L2 comprising the amino acid sequence of SASSLYS.

204. The polypeptide of claim 202 or 203, wherein the VL comprises a CDR-L1 comprising the amino acid sequence of RASQSVSSAVA.

205. The polypeptide of any one of claims 199-204, wherein the antigen binding domain comprises:
a CDR-H3 sequence of GGVIHAMDY,
a CDR-H2 sequence of SILSRWGVTSYADSVKG,
a CDR-H1 sequence of FSPYSIH,
a CDR-L3 sequence of QQASYVRKTIT,
a CDR-L2 sequence of SASSLYS, and
a CDR-L1 sequence of RASQSVSSAVA.

206. The polypeptide of any one of claims 199-205, wherein the VH comprises a sequence with at least 80% sequence identity to the sequence
EVQLVESGGGLVQPGGSLRLSCAASGFTFSPYSIHWVRQAPGKGLEWVAS
ILSRWGVTSYADSVKGRFTISADTSKNTAYLQMNSLRAEDTAVYYCARGG
VIHAMDYWGQGTLVTVSS.

207. The polypeptide of any one of claims 202-205, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

208. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GSWIAAMDY.

209. The polypeptide of claim 208, wherein the VH comprises a CDR-H2 comprising the amino acid sequence of SISSWHGETGYADSVKG.

210. The polypeptide of claim 208 or 209, wherein the VH comprises a CDR-H1 comprising the amino acid sequence of FSPYSIH.

211. The polypeptide of any one of claims 208-210, wherein the antigen-binding domain further comprises a light chain variable region (VL), and wherein the VL comprises a CDR-L3 comprising the amino acid sequence of QQASYVRKTIT.

212. The polypeptide of claim 211, wherein the VL comprises a CDR-L2 comprising the amino acid sequence of SASSLYS.

213. The polypeptide of claim 211 or 212, wherein the VL comprises a CDR-L1 comprising the amino acid sequence of RASQSVSSAVA.

214. The polypeptide of any one of claims 208-213, wherein the antigen binding domain comprises:
a CDR-H3 sequence of GSWIAAMDY,
a CDR-H2 sequence of SISSWHGETGYADSVKG,
a CDR-H1 sequence of FSPYSIH,
a CDR-L3 sequence of QQASYVRKTIT,
a CDR-L2 sequence of SASSLYS, and
a CDR-L1 sequence of RASQSVSSAVA.

215. The polypeptide of any one of claims 208-214, wherein the VH comprises a sequence with at least 80% sequence identity to the sequence
EVQLVESGGGLVQPGGSLRLSCAASGFTFSPYSIHWVRQAPGKGL
EWVASISSWHGETGYADSVKGRFTISADTSKNTAYLQMNSLRAED
TAVYYCARGSWIAAMDYWGQGTLVTVSS.

216. The polypeptide of any one of claims 211-214, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

217. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GGWIAAMDY.

218. The polypeptide of claim 217, wherein the VH comprises a CDR-H2 comprising the amino acid sequence of SISSLQGDTGYADSVKG.

219. The polypeptide of claim 217 or 218, wherein the VH comprises a CDR-H1 comprising the amino acid sequence of FSWYSIH.

220. The polypeptide of any one of claims 217-219, wherein the antigen-binding domain further comprises a light chain variable region (VL), and wherein the VL comprises a CDR-L3 comprising the amino acid sequence of QQASYVRKTIT.

221. The polypeptide of claim 220, wherein the VL comprises a CDR-L2 comprising the amino acid sequence of SASSLYS.

222. The polypeptide of claim 220 or 221, wherein the VL comprises a CDR-L1 comprising the amino acid sequence of RASQSVSSAVA.

223. The polypeptide of any one of claims 217-222, wherein the antigen binding domain comprises:
a CDR-H3 sequence of GGWIAAMDY,
a CDR-H2 sequence of SISSLQGDTGYADSVKG,
a CDR-H1 sequence of FSWYSIH,
a CDR-L3 sequence of QQASYVRKTIT,
a CDR-L2 sequence of SASSLYS, and
a CDR-L1 sequence of RASQSVSSAVA.

224. The polypeptide of any one of claims 217-223, wherein the VH comprises a sequence with at least 80% sequence identity to the sequence
EVQLVESGGGLVQPGGSLRLSCAASGFTFSWYSIHWVRQAPGKGL
EWVASISSLQGDTGYADSVKGRFTISADTSKNTAYLQMNSLRAED
TAVYYCARGGWIAAMDYWGQGTLVTVSS.

225. The polypeptide of any one of claims 220-223, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

226. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GSWIAAMDY.

227. The polypeptide of claim 226, wherein the VH comprises a CDR-H2 comprising the amino acid sequence of SIASWYGDTGYADSVKG.

228. The polypeptide of claim 226 or 227, wherein the VH comprises a CDR-H1 comprising the amino acid sequence of FHYYSIH.

229. The polypeptide of any one of claims 226-228, wherein the antigen-binding domain further comprises a light chain variable region (VL), and wherein the VL comprises a CDR-L3 comprising the amino acid sequence of QQASYVRKTIT.

230. The polypeptide of claim 229, wherein the VL comprises a CDR-L2 comprising the amino acid sequence of SASSLYS.

231. The polypeptide of claim 229 or 230, wherein the VL comprises a CDR-L1 comprising the amino acid sequence of RASQSVSSAVA.

232. The polypeptide of any one of claims 229-231, wherein the antigen binding domain comprises:
a CDR-H3 sequence of GSWIAAMDY,
a CDR-H2 sequence of SIASWYGDTGYADSVKG,
a CDR-H1 sequence of FHYYSIH,
a CDR-L3 sequence of QQASYVRKTIT,
a CDR-L2 sequence of SASSLYS, and
a CDR-L1 sequence of RASQSVSSAVA.

233. The polypeptide of any one of claims 226-232, wherein the VH comprises a sequence with at least 80% sequence identity to the sequence
EVQLVESGGGLVQPGGSLRLSCAASGFTFHYYSIHWVRQAPGKGL
EWVASIASWYGDTGYADSVKGRFTISADTSKNTAYLQMNSLRAED
TAVYYCARGSWIAAMDYWGQGTLVTVSS.

234. The polypeptide of any one of claims 229-232, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

235. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GGRIEAMDY.

236. The polypeptide of claim 235, wherein the VH comprises a CDR-H2 comprising the amino acid sequence of SISSWYGKTGYADSVKG.

237. The polypeptide of claim 235 or 236, wherein the VH comprises a CDR-H1 comprising the amino acid sequence of FGYYSIH.

238. The polypeptide of any one of claims 235-237, wherein the antigen-binding domain further comprises a light chain variable region (VL), and wherein the VL comprises a CDR-L3 comprising the amino acid sequence of QQASYVRKTIT.

239. The polypeptide of claim 238, wherein the VL comprises a CDR-L2 comprising the amino acid sequence of SASSLYS.

240. The polypeptide of claim 238 or 239, wherein the VL comprises a CDR-L1 comprising the amino acid sequence of RASQSVSSAVA.

241. The polypeptide of any one of claims 238-240, wherein the antigen binding domain comprises:
a CDR-H3 sequence of GGRIEAMDY,
a CDR-H2 sequence of SISSWYGKTGYADSVKG,
a CDR-H1 sequence of FGYYSIH,
a CDR-L3 sequence of QQASYVRKTIT,
a CDR-L2 sequence of SASSLYS, and
a CDR-L1 sequence of RASQSVSSAVA.

242. The polypeptide of any one of claims 236-241, wherein the VH comprises a sequence with at least 80% sequence identity to the sequence
EVQLVESGGGLVQPGGSLRLSCAASGFTFGYYSIHWVRQAPGKGL
EWVASISSWYGKTGYADSVKGRFTISADTSKNTAYLQMNSLRAED
TAVYYCARGGRIEAMDYWGQGTLVTVSS.

243. The polypeptide of any one of claims 238-241, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

244. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GYWIEAMDY.

245. The polypeptide of claim 244, wherein the VH comprises a CDR-H2 comprising the amino acid sequence of SIASSYGSTGYADSVKG.

246. The polypeptide of claim 244 or 245, wherein the VH comprises a CDR-H1 comprising the amino acid sequence of FSKYSIH.

247. The polypeptide of any one of claims 244-246, wherein the antigen-binding domain further comprises a light chain variable region (VL), and wherein the VL comprises a CDR-L3 comprising the amino acid sequence of QQASYVRKTIT.

248. The polypeptide of claim 247, wherein the VL comprises a CDR-L2 comprising the amino acid sequence of SASSLYS.

249. The polypeptide of claim 247 or 248, wherein the VL comprises a CDR-L1 comprising the amino acid sequence of RASQSVSSAVA.

250. The polypeptide of any one of claims 247-249, wherein the antigen binding domain comprises:
a CDR-H3 sequence of GYWIEAMDY,
a CDR-H2 sequence of SIASSYGSTGYADSVKG,
a CDR-H1 sequence of FSKYSIH,
a CDR-L3 sequence of QQASYVRKTIT,
a CDR-L2 sequence of SASSLYS, and
a CDR-L1 sequence of RASQSVSSAVA.

251. The polypeptide of any one of claims 244-250, wherein the VH comprises a sequence with at least 80% sequence identity to the sequence
EVQLVESGGGLVQPGGSLRLSCAASGFTFSKYSIHWVRQAPGKGL
EWVASIASSYGSTGYADSVKGRFTISADTSKNTAYLQMNSLRAED
TAVYYCARGYWIEAMDYWGQGTLVTVSS.

252. The polypeptide of any one of claims 247-250, wherein the VL comprises a sequence with at least 80% sequence identity to the sequence
DIQMTQSPSSLSASVGDRVTITCRASQSVSSAVAWYQQKPGKAPK
LLIYSASSLYSGVPSRFSGSRSGTDFTLTISSLQPEDFATYYCQQ
ASYVRKTITFGQGTKVEIKRTV.

253. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GSWIAAMDY.

254. The polypeptide of claim 253, wherein the VH comprises a CDR-H2 comprising the amino acid sequence of SIHSSIGTTGYADSVKG.

255. The polypeptide of claim 253 or 254, wherein the VH comprises a CDR-H1 comprising the amino acid sequence of FGLYSIH.

256. The polypeptide of any one of claims 253-255, wherein the antigen-binding domain further comprises a light chain variable region (VL), and wherein the VL comprises a CDR-L3 comprising the amino acid sequence of QQASYVRKTIT.

257. The polypeptide of claim 256, wherein the VL comprises a CDR-L2 comprising the amino acid sequence of SASSLYS.

258. The polypeptide of claim 256 or 257, wherein the VL comprises a CDR-L1 comprising the amino acid sequence of RASQSVSSAVA.

259. The polypeptide of any one of claims 256-258, wherein the antigen binding domain comprises:
a CDR-H3 sequence of GSWIAAMDY,
a CDR-H2 sequence of SIHSSIGTTGYADSVKG,
a CDR-H1 sequence of FGLYSIH,
a CDR-L3 sequence of QQASYVRKTIT,
a CDR-L2 sequence of SASSLYS, and
a CDR-L1 sequence of RASQSVSSAVA.

260. The polypeptide of any one of claims 253-259, wherein the VH comprises a sequence with at least 80% sequence identity to the sequence
EVQLVESGGGLVQPGGSLRLSCAASGFTFGLYSIHWVRQAPGKGL
EWVASIHSSIGTTGYADSVKGRFTISADTSKNTAYLQMNSLRAED
TAVYYCARGSWIAAMDYWGQGTLVTVSS.

261. The polypeptide of any one of claims 256-259, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

262. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GSVIHAMDY.

263. The polypeptide of claim 262, wherein the VH comprises a CDR-H2 comprising the amino acid sequence of SILSWIGKTSYADSVKG.

264. The polypeptide of claim 262 or 263, wherein the VH comprises a CDR-H1 comprising the amino acid sequence of FSPYSIH.

265. The polypeptide of any one of claims 262-264, wherein the antigen-binding domain further comprises a light chain variable region (VL), and wherein the VL comprises a CDR-L3 comprising the amino acid sequence of QQASYVRKTIT.

266. The polypeptide of claim 265, wherein the VL comprises a CDR-L2 comprising the amino acid sequence of SASSLYS.

267. The polypeptide of claim 265 or 266, wherein the VL comprises a CDR-L1 comprising the amino acid sequence of RASQSVSSAVA.

268. The polypeptide of any one of claims 262-267, wherein the antigen binding domain comprises:
a CDR-H3 sequence of GSVIHAMDY,
a CDR-H2 sequence of SILSWIGKTSYADSVKG,
a CDR-H1 sequence of FSPYSIH,
a CDR-L3 sequence of QQASYVRKTIT,
a CDR-L2 sequence of SASSLYS, and
a CDR-L1 sequence of RASQSVSSAVA.

269. The polypeptide of any one of claims 262-268, wherein the VH comprises a sequence with at least 80% sequence identity to the sequence
EVQLVESGGGLVQPGGSLRLSCAASGFTFSPYSIHWVRQAPGKGLEWVAS
ILSWIGKTSYADSVKGRFTISADTSKNTAYLQMNSLRAEDTAVYYCARGS
VIHAMDYWGQGTLVTVSS.

270. The polypeptide of any one of claims 265-268, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

271. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GGWIAAMDY.

272. The polypeptide of claim 271, wherein the VH comprises a CDR-H2 comprising the amino acid sequence of SIASRWGHTGYADSVKG.

273. The polypeptide of claim 271 or 272, wherein the VH comprises a CDR-H1 comprising the amino acid sequence of FSPYHIH.

274. The polypeptide of any one of claims 271-273, wherein the antigen-binding domain further comprises a light chain variable region (VL), and wherein the VL comprises a CDR-L3 comprising the amino acid sequence of QQASYVRKTIT.

275. The polypeptide of claim 274, wherein the VL comprises a CDR-L2 comprising the amino acid sequence of SASSLYS.

276. The polypeptide of claim 274 or 275, wherein the VL comprises a CDR-L1 comprising the amino acid sequence of RASQSVSSAVA.

277. The polypeptide of any one of claims 274-276, wherein the antigen binding domain comprises:
a CDR-H3 sequence of GGWIAAMDY,
a CDR-H2 sequence of SIASRWGHTGYADSVKG,
a CDR-H1 sequence of FSPYHIH,
a CDR-L3 sequence of QQASYVRKTIT,
a CDR-L2 sequence of SASSLYS, and
a CDR-L1 sequence of RASQSVSSAVA.

278. The polypeptide of any one of claims 271-277, wherein the VH comprises a sequence with at least 80% sequence identity to the sequence
EVQLVESGGGLVQPGGSLRLSCAASGFTFSPYHIHWVRQAPGKGLEWVAS
IASRWGHTGYADSVKGRFTISADTSKNTAYLQMNSLRAEDTAVYYCARGG
WIAAMDYWGQGTLVTVSS.

279. The polypeptide of any one of claims 274-277, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

280. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of GSWIAAMDY.

281. The polypeptide of claim 280, wherein the VH comprises a CDR-H2 comprising the amino acid sequence of SIASLQGITGYADSVKG.

282. The polypeptide of claim 280 or 281, wherein the VH comprises a CDR-H1 comprising the amino acid sequence of FHEYSIH.

283. The polypeptide of any one of claims 280-282, wherein the antigen-binding domain further comprises a light chain variable region (VL), and wherein the VL comprises a CDR-L3 comprising the amino acid sequence of QQASYVRKTIT.

284. The polypeptide of claim 283, wherein the VL comprises a CDR-L2 comprising the amino acid sequence of SASSLYS.

285. The polypeptide of claim 283 or 284, wherein the VL comprises a CDR-L1 comprising the amino acid sequence of RASQSVSSAVA.

286. The polypeptide of any one of claims 280-285, wherein the antigen binding domain comprises:
a CDR-H3 sequence of GSWIAAMDY,
a CDR-H2 sequence of SIASLQGITGYADSVKG,
a CDR-H1 sequence of FHEYSIH,
a CDR-L3 sequence of QQASYVRKTIT,
a CDR-L2 sequence of SASSLYS, and
a CDR-L1 sequence of RASQSVSSAVA.

287. The polypeptide of any one of claims 280-286, wherein the VH comprises a sequence with at least 80% sequence identity to the sequence
EVQLVESGGGLVQPGGSLRLSCAASGFTFHEYSIHWVRQAPGKGLEWVAS
IASLQGITGYADSVKGRFTISADTSKNTAYLQMNSLRAEDTAVYYCARGS
WIAAMDYWGQGTLVTVSS.

288. The polypeptide of any one of claims 283-286, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

289. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GGVIHAMDY.

290. The polypeptide of claim 289, wherein the VH comprises a CDR-H2 comprising the amino acid sequence of SILSRWGVTSYADSVKG.

291. The polypeptide of claim 289 or 290, wherein the VH comprises a CDR-H1 comprising the amino acid sequence of FSDYSIH.

292. The polypeptide of any one of claims 289-291, wherein the antigen-binding domain further comprises a light chain variable region (VL), and wherein the VL comprises a CDR-L3 comprising the amino acid sequence of QQASYVRKTIT.

293. The polypeptide of claim 292, wherein the VL comprises a CDR-L2 comprising the amino acid sequence of SASSLYS.

294. The polypeptide of claim 292 or 293, wherein the VL comprises a CDR-L1 comprising the amino acid sequence of RASQSVSSAVA.

295. The polypeptide of any one of claims 289-294, wherein the antigen binding domain comprises:
a CDR-H3 sequence of GGVIHAMDY,
a CDR-H2 sequence of SILSRWGVTSYADSVKG,
a CDR-H1 sequence of FSDYSIH,
a CDR-L3 sequence of QQASYVRKTIT,
a CDR-L2 sequence of SASSLYS, and
a CDR-L1 sequence of RASQSVSSAVA.

296. The polypeptide of any one of claims 289-295, wherein the VH comprises a sequence with at least 80% sequence identity to the sequence
EVQLVESGGGLVQPGGSLRLSCAASGFTFSDYSIHWVRQAPGKGLEWVAS
ILSRWGVTSYADSVKGRFTISADTSKNTAYLQMNSLRAEDTAVYYCARGG
VIHAMDYWGQGTLVTVSS.

297. The polypeptide of any one of claims 292-295, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

298. The polypeptide of any one of claims 167-173, wherein the antigen-binding domain comprises a heavy chain variable region (VH), and wherein the VH comprises a heavy chain complementarity determining region 3 (CDR-H3) comprising the amino acid sequence of
GGVIHAMDY.

299. The polypeptide of claim 298, wherein the VH comprises a CDR-H2 comprising the amino acid sequence of SISSRWGVTSYADSVKG.

300. The polypeptide of claim 298 or 299, wherein the VH comprises a CDR-H1 comprising the amino acid sequence of FSDYSIH.

301. The polypeptide of any one of claims 298-300, wherein the antigen-binding domain further comprises a light chain variable region (VL), and wherein the VL comprises a CDR-L3 comprising the amino acid sequence of QQASYVRKTIT.

302. The polypeptide of claim 301, wherein the VL comprises a CDR-L2 comprising the amino acid sequence of SASSLYS.

303. The polypeptide of claim 301 or 302, wherein the VL comprises a CDR-L1 comprising the amino acid sequence of RASQSVSSAVA.

304. The polypeptide of any one of claims 298-303, wherein the antigen binding domain comprises:
a CDR-H3 sequence of GGVIHAMDY,
a CDR-H2 sequence of SISSRWGVTSYADSVKG,
a CDR-H1 sequence of FSDYSIH,
a CDR-L3 sequence of QQASYVRKTIT,
a CDR-L2 sequence of SASSLYS, and
a CDR-L1 sequence of RASQSVSSAVA.

305. The polypeptide of any one of claims 298-304, wherein the VH comprises a sequence with at least 80% sequence identity to the sequence
EVQLVESGGGLVQPGGSLRLSCAASGFTFSDYSIHWVRQAPGKGLEWVAS
ISSRWGVTSYADSVKGRFTISADTSKNTAYLQMNSLRAEDTAVYYCARGG
VIHAMDYWGQGTLVTVSS.

306. The polypeptide of any one of claims 301-304, wherein the VL comprises a sequence with at least 80% sequence identity to the sequence
DIQMTQSPSSLSASVGDRVTITCRASQSVSSAVAWYQQKPGKAPKLLIYS
ASSLYSGVPSRFSGSRSGTDFTLTISSLQPEDFATYYCQQASYVRKTITF
GQGTKVEIKRTV.

307. The polypeptide of any one of claims 166-180, wherein the antibody or the antigen-binding fragment specifically binds to a peptide conjugate/MHC complex and wherein the antibody or the antigen-binding fragment interacts with the MHC of the peptide conjugate/MHC complex.

308. The polypeptide of claim 307, wherein the peptide conjugate/MHC complex comprises:
(a) a peptide conjugate comprising a peptide covalently linked to a targeted covalent inhibitor or a fragment thereof; and
(b) an MHC.

309. The polypeptide of claim 308, wherein the MHC is a human leukocyte antigen (HLA).

310. The polypeptide of claim 309, wherein the HLA is HLA-A*03:01, HLA-A*11:01, and/or HLA-A*02:01.

311. The polypeptide of any one of claims 308-310, wherein the antigen-binding domain binds to the peptide conjugate/MHC complex with a greater affinity than to the peptide or free targeted covalent inhibitor.

312. The polypeptide of any one of claims 307-311, wherein the antigen-binding domain binds to the peptide conjugate/MHC complex with a dissociation constant (K_{D}) of at most about 50 nM, at most about 40 nM, at most about 30 nM, at most about 20 nM, at most about 10 nM, at most about 1 nM, at most about 0.1 nM, at most about 10 pM, at most about 1 pM, or at most about 0.1 pM.

313. The polypeptide of any one of claims 307-312, wherein the antigen-binding domain binds to the peptide conjugate/MHC complex with a dissociation constant (K_{D}) of from about 0.01 nM to about 20 nM.

314. The polypeptide of any one of claims 308-313, wherein the antigen-binding domain binds to the free targeted covalent inhibitor with a dissociation constant (K_{D}) of at least about 100 nM, at least about 200 nM, at least about 300 nM, at least about 400 nM, at least about 500 nM, at least about 1 μM, at least about 10 μM, at least about 20 μM, at least about 30 μM, at least about 40 μM, at least about 50 M, or at least 100 μM.

315. The polypeptide of any one of claims 308-313, wherein the antigen-binding domain binds to the free targeted covalent inhibitor with a dissociation constant (K_{D}) of at least about 10, 100, 10,000, or 100,000 times more than a K_{D} of the antibody or the antigen-binding fragment binding to the peptide conjugate/MHC complex.

316. The polypeptide of any one of claims 308-315, wherein the antigen-binding domain does not detectably bind to the free targeted covalent inhibitor.

317. The polypeptide of any one of claims 308-315, wherein the antigen-binding domain binds to the free targeted covalent inhibitor with an IC₅₀ of at least about 50 nM.

318. The polypeptide of any one of claims 308-315, wherein the antigen-binding domain binds to the free peptide conjugate with a dissociation constant (K_{D}) of more than about 100 nM, more than about 200 nM, more than about 300 nM, more than about 400 nM, more than about 500 nM, more than 1 μM, more than 10 μM, more than 20 μM, more than 30 pM, more than 40 μM, more than 50 μM, or more than 100 μM.

319. The polypeptide of any one of claims 308-318, wherein the antigen-binding domain binds to the free peptide conjugate with a dissociation constant (K_{D}) that is at least about 10, 100, 10,000, 100,000 times more than a K_{D} of the antibody or the antigen-binding fragment to the peptide conjugate/MHC complex.

320. The polypeptide of any one of claims 308-318, wherein the antigen-binding domain binds to the free peptide conjugate with a dissociation constant (K_{D}) that is at least 2.5 times more than a K_{D} of the antibody or the antigen-binding fragment to the peptide conjugate/MHC complex.

321. The polypeptide of claim 320, wherein the antigen-binding domain binds to the free peptide conjugate with a dissociation constant (K_{D}) that is higher than a K_{D} of the antibody or the antigen-binding fragment to the peptide conjugate/MHC complex, wherein the MHC is HLA-A02:01.

322. The polypeptide of claim 320, wherein the antigen-binding domain binds to the free peptide conjugate with a dissociation constant (K_{D}) that is at least 2.5 times more than a K_{D} of the antibody or the antigen-binding fragment to the peptide conjugate/MHC complex, wherein the MHC is HLA-A03:01.

323. The polypeptide of any one of claims 308-319, wherein the antigen-binding domain binds to the peptide conjugate/MHC complex with an affinity that is at least 100 times, at least 200 times, at least 300 times, at least 400 times, at least 500 times, at least 600 times, at least 700 times, at least 800 times, at least 900 times, at least 1,000 times, at least 2,500 times, at least 5,000 times, or at least 10,000 times greater than the affinity of antibody or the antigen-binding fragment to the free targeted covalent inhibitor or the free peptide conjugate.

324. The polypeptide of any one of claims 308-323, wherein the peptide conjugate is formed by the covalent reaction of a free targeted covalent inhibitor with a KRAS^{G12C} peptide, a KRAS^{G12D} peptide, a KRAS^{G12R} peptide, or a KRAS^{G12S} peptide.

325. The polypeptide of claim 324, wherein the free targeted covalent inhibitor is osimertinib, ibrutinib, neratinib, sotorasib, ARS-853, ARS-1620, ARS-3248, MRTX849, JNJ74699157, LY3499446, LY3537982, MRTX-1257, JDQ443, MRTX-1133, RMC-6291, RMC-9805, GDC-6036, D-1553, 2E07, 6H05, SML-8-73-1, or BI 182391.

326. The polypeptide of claim 325, wherein the peptide conjugate is formed by the covalent reaction of sotorasib with a KRAS^{G12C} peptide.

327. The polypeptide of claim 324, wherein the peptide comprises or consists of the amino acid sequence of VVVGACGVGK, VVGACGVGK, or KLVVVGACGV.

328. The polypeptide of any one of claims 166-327, wherein the antigen binding domain (i) has specificity to a peptide conjugate/MHC complex comprising VVGACGVGK conjugated to a targeted covalent inhibitor or fragment thereof presented by HLA-A*11:01 and/or a peptide conjugate/MHC complex comprising VVVGACGVGK conjugated to a targeted covalent inhibitor or fragment thereof presented by HLA-A*11:01, or (ii) has specificity to a peptide conjugate/MHC complex comprising VVGACGVGK conjugated to a targeted covalent inhibitor or fragment thereof presented by HLA-A*03:01 and/or a peptide conjugate/MHC complex comprising VVVGACGVGK conjugated to a targeted covalent inhibitor or fragment thereof presented by HLA-A*03:01.

329. The polypeptide of any one of claims 166-327, wherein the antigen binding domain has specificity to a peptide conjugate/MHC complex comprising VVVGACGVGK conjugated to a targeted covalent inhibitor or fragment thereof presented by HLA-A*03:01 and/or a peptide conjugate/MHC complex comprising VVVGACGVGK conjugated to a targeted covalent inhibitor or fragment thereof presented by HLA-A*11:01.

330. The polypeptide of any one of claims 166-327, wherein the antigen binding domain has specificity to a peptide conjugate/MHC complex comprising VVGACGVGK conjugated to a targeted covalent inhibitor or fragment thereof presented by HLA-A*11:01, a peptide conjugate/MHC complex comprising VVGACGVGK conjugated to a targeted covalent inhibitor or fragment thereof presented by HLA-A*03:01, a peptide conjugate/MHC complex comprising VVVGACGVGK conjugated to a targeted covalent inhibitor or fragment thereof presented by HLA-A*03:01, a peptide conjugate/MHC complex comprising VVVGACGVGK conjugated to a targeted covalent inhibitor or fragment thereof presented by HLA-A*11:01, and/or a peptide conjugate/MHC complex comprising KLVVVGACGV conjugated to a targeted covalent inhibitor or fragment thereof presented by HLA-A*02:01.

331. The polypeptide of any one of claims 166-330, wherein the polypeptide binds to:
(i) a peptide conjugate/HLA-A*11:01 MHC complex containing a peptide consisting of the amino acid sequence VVGACGVGK and
a peptide conjugate/HLA-A*11:01 MHC complex containing a peptide consisting of the amino acid sequence VVVGACGVGK;
(ii) a peptide conjugate/HLA-A*03:01 MHC complex containing a peptide consisting of the amino acid sequence VVGACGVGK and
a peptide conjugate/HLA-A*03:01 MHC complex containing a peptide consisting of the amino acid sequence VVVGACGVGK;
(iii) a peptide conjugate/HLA-A*03:01 MHC complex containing a peptide consisting of the amino acid sequence VVGACGVGK and
a peptide conjugate/HLA-A*11:01 MHC complex containing a peptide consisting of the amino acid sequence VVGACGVGK;
(iv) a peptide conjugate/HLA-A*03:01 MHC complex containing a peptide consisting of the amino acid sequence VVVGACGVGK and
a peptide conjugate/HLA-A*11:01 MHC complex containing a peptide consisting of the amino acid sequence VVVGACGVGK; and/or
(v) a peptide conjugate/HLA-A*02:01 MHC complex containing a peptide consisting of the amino acid sequence KLVVVGACGV.

332. The polypeptide of any one of claims 166-330, wherein the polypeptide binds to: a peptide conjugate/HLA-A*03:01 MHC complex containing a peptide consisting of the amino acid sequence VVGACGVGK, and a peptide conjugate/HLA-A*03:01 MHC complex containing a peptide consisting of the amino acid sequence VVVGACGVGK, and a peptide conjugate/HLA-A*11:01 MHC complex containing a peptide consisting of the amino acid sequence VVGACGVGK, and a peptide conjugate/HLA-A*11:01 MHC complex containing a peptide consisting of the amino acid sequence VVVGACGVGK, and a peptide conjugate/HLA-A*02:01 MHC complex containing a peptide consisting of the amino acid sequence KLVVVGACGV.

333. The polypeptide of any one of claims 308-327, wherein an interface area of the polypeptide with the peptide conjugate/MHC complex is at least about 500 Å², 600 Å², 700 Å², 800 Å², 900 Å², 1,000 Å², 1,200 Å², 1,500 Å², or 2,000 Å².

334. The polypeptide of any one of claims 308-333, wherein the interface area of the polypeptide with the MHC of the peptide conjugate/MHC complex is more than the interface area of the polypeptide with the peptide or the targeted covalent inhibitor.

335. The polypeptide of any one of claims 308-334, wherein the polypeptide forms a binding pocket at the interface between VH and VL domains to accommodate the targeted covalent inhibitor of the peptide conjugate/MHC complex.

336. The polypeptide of any one of claims 308-335, wherein the polypeptide does not bind to the peptide conjugate/MHC complex with a head-to-head coaxial interaction.

337. The polypeptide of any one of claims 308-336, wherein:
(a) the polypeptide binds to the peptide conjugate/MHC complex at an angle from about 10° to 60° between the axis of the MHC and the axis of the polypeptide;
(b) when the polypeptide is bound to the peptide conjugate/MHC complex, the angle between the axis of the polypeptide and the axis of the peptide conjugate/MHC complex is from about 10° to 60°;
(c) when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex;
(d) when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex;
(e) the polypeptide binds to the peptide conjugate/MHC complex at an angle that is different than the angle to which a T cell receptor binds to the peptide conjugate/MHC complex; and/or
(f) the polypeptide binds to the peptide conjugate/MHC complex at an angle that is from about 10° to 60° less or from about 10° to 60° more than the angle to which a T cell receptor binds to the peptide conjugate/MHC complex.

338. The polypeptide of any one of claims 308-337, wherein
(a) the polypeptide binds to the peptide conjugate/MHC complex at an angle of about 40° between the axis of the MHC and the axis of the polypeptide;
(b) when the polypeptide is bound to the peptide conjugate/MHC complex, the angle between the axis of the polypeptide and the axis of the peptide conjugate/MHC complex is about 40°;
(c) when the polypeptide is bound to the peptide conjugate/MHC complex, the angle between the axis of the polypeptide and the axis of the peptide conjugate/MHC complex is about 40° less or about 40° more than the angle between the axis of a TCR and the axis of the peptide conjugate/MHC complex when the TCR is bound to the peptide conjugate/MHC complex; and/or
(d) the polypeptide binds to the peptide conjugate/MHC complex at an angle that is about 40° less or about 40° more than the angle to which a T cell receptor binds to the peptide conjugate/MHC complex.

339. The polypeptide of any one of claims 308-338, wherein the polypeptide contacts the alpha 1 domain and alpha 2 domain of a heavy chain of the MHC of the peptide conjugate/MHC complex.

340. The polypeptide of any one of claims 308-339, wherein the polypeptide binds to an epitope of the MHC, and wherein the epitope comprises one or more residues from the regions comprising residues 62-66, 106-109, and/or 150-170 of the MHC.

341. The polypeptide of claim 340, wherein the polypeptide binds to an epitope of the MHC, and wherein the epitope comprises one or more residues from the regions comprising residues 62-66, 106-109, and/or 150-170 of the HLA-A*03:01 or the HLA-A*11:01.

342. The polypeptide of claim 340 or 341, wherein the polypeptide binds to an epitope of the MHC, and wherein the epitope comprises one or more residues selected from the group consisting of residues 62, 106, 108, 109, 158, 161, 162, 162, 165, 166, 167, 169 and 170 of the HLA-A*03:01.

343. The polypeptide of claim 342, wherein the VL domain of the antigen-binding domain binds to an epitope of the MHC, and wherein the epitope comprises one or more residues selected from the group consisting of residues 62, 106, 108, 109, 158, 161, 162, 162, 165, 166, 167, 169 and 170 of the HLA-A*03:01.

344. The polypeptide of any one of claims 340-343, wherein the polypeptide binds to an epitope of the MHC, and wherein the epitope comprises one or more residues selected from the group consisting of residues 62, 65, 66, 150, 151, 154, 155, 157, and 158 of the HLA-A*03:01.

345. The polypeptide of claim 344, wherein the VH domain of the antigen-binding domain binds to an epitope of the MHC, and wherein the epitope comprises one or more residues selected from the group consisting of residues 62, 65, 66, 150, 151, 154, 155, 157, and 158 of the HLA-A*03:01.

346. The polypeptide of claim 340 or 341, wherein the polypeptide binds to an epitope of the MHC, and wherein the epitope comprises one or more residues selected from the group consisting of residues 62, 106, 108, 109, 154, 157, 158, 161, 162, 163, 165, 166, 167, 169 and 170 of the HLA-A*11:01.

347. The polypeptide of claim 346, wherein the VL domain of the antigen-binding domain binds to an epitope of the MHC, and wherein the epitope comprises one or more residues selected from the group consisting of residues 62, 106, 108, 109, 154, 157, 158, 161, 162, 163, 165, 166, 167, 169 and 170 of the HLA-A*11:01.

348. The polypeptide of claim 346 or 347, wherein the polypeptide binds to an epitope of the MHC, and wherein the epitope comprises one or more residues selected from the group consisting of residues 62, 65, 66, 151, 154, 155 and 158 of the HLA-A*11:01.

349. The polypeptide of claim 348, wherein the VH domain of the antigen-binding domain binds to an epitope of the MHC, and wherein the epitope comprises one or more residues selected from the group consisting of residues 62, 65, 66, 151, 154, 155 and 158 of the HLA-A*11:01.

350. The polypeptide of any one of claims 308-349, wherein the VH is linked to the VL through a linker.

351. The polypeptide of claim 350, wherein the linker comprises (G₄S)ₙ or (S₄G)ₙ where n is any integer from 1 to 10.

352. The polypeptide of claim 351, wherein the linker comprises the glycine-serine-alanine linker G₄SA₃ or a glycine-serine linker (G₄S)₄.

353. The polypeptide of any one of claims 166-352, wherein the polypeptide is an intact antibody, a bispecific antibody, a multispecific antibody, an antigen-binding (Fab) fragment, an Fab′ fragment, an (Fab′)2 fragment, an Fd, an Fv, a dAb, a single domain fragment or single monomeric variable antibody domain, a Dual-Affinity Retargeting (DART) molecule, a Diabody (Db), a single-chain Diabody (scDb), a single-chain variable fragment (scFv), a bispecific T-cell engager (BiTE), bispecific killer cell engager (BiKE), CrossMab, a camelid antibody, a tri-specific binding partner, a chimeric antigen receptor (CAR), a Monobody (aka Adnectin), a DARPin, an anticalin, an affibody, or an affimer.

354. The polypeptide of claim 353, wherein the polypeptide is a bispecific antibody.

355. The polypeptide of claim 354, wherein the bispecific antibody is a bispecific T-cell engager (BiTE).

356. The polypeptide of any one of claims 166-355, wherein polypeptide further comprises a second antigen-binding domain that binds to a T cell surface marker.

357. The polypeptide of claim 356, wherein the T cell surface marker is CD3 epsilon, CD3 gamma, CD3 delta, CD3 eta, a TCR alpha, or a TCR beta of a TCR.

358. The polypeptide of claim 356 or 357, wherein the antigen-binding domain and the second antigen-binding domain is linked by a linker.

359. The polypeptide of claim 358, wherein the linker comprises (G₄S)ₙ or (S₄G)ₙ where n is any integer from 1 to 10.

360. The polypeptide of claims 358 and 359, wherein the linker is configured such that:
(a) the polypeptide binds to the peptide conjugate/MHC complex at an angle from about 10° to 60° between the axis of the MHC and the axis of the polypeptide;
(b) when the polypeptide is bound to the peptide conjugate/MHC complex, the angle between the axis of the polypeptide and the axis of the peptide conjugate/MHC complex is from about 10° to 60°;
(c) when the polypeptide is bound to the peptide conjugate/MHC complex, the angle between the axis of the polypeptide and the axis of the peptide conjugate/MHC complex is different than the angle between the axis of a TCR and the axis of the peptide conjugate/MHC complex when the TCR is bound to the peptide conjugate/MHC complex;
(d) when the polypeptide is bound to the peptide conjugate/MHC complex, the angle between the axis of the polypeptide and the axis of the peptide conjugate/MHC complex is from about 10° to 60° less or from about 10° to 60° more than the angle between the axis of a TCR and the axis of the peptide conjugate/MHC complex when the TCR is bound to the peptide conjugate/MHC complex;
(e) the polypeptide binds to the peptide conjugate/MHC complex at an angle that is different than the angle to which a T cell receptor binds to the peptide conjugate/MHC complex; and/or
(f) the polypeptide binds to the peptide conjugate/MHC complex at an angle that is from about 10° to 60° less or from about 10° to 60° more than the angle to which a T cell receptor binds to the peptide conjugate/MHC complex.

361. The polypeptide of any one of claims 356-360, wherein the polypeptide comprises a first polypeptide chain comprising the antigen-binding domain and a second polypeptide chain comprising the second antigen-binding domain.

362. The polypeptide of claim 361, wherein the first or the second polypeptide chain is further fused to a cytokine or fragment thereof.

363. The polypeptide of claim 362, wherein the cytokine comprises IL-2, IL-7, IL-15, IL-12, IL-18, or IL-21, or an interferon (IFN).

364. The polypeptide of any one of claims 307-363, wherein the peptide conjugate/MHC complex is presented on a surface of a cell.

365. The polypeptide of claim 364, wherein the cell expresses a low copy number of the peptide conjugate/MHC complex, and wherein the low copy number is at most about 1000, 900, 800, 700, 600, 500, 400, 300, 200, 100, 90, 80, 70, 60, 50, 40, 30, 20, 10, 5, 3, or 1 copy per a single cell.

366. The polypeptide of claim 364 or 365, wherein the peptide of the peptide conjugate/MHC complex is from an intracellular protein.

367. The polypeptide of any one of claims 166-366, wherein the polypeptide is the binding partner of any one of the preceding claims.

368. A polypeptide comprising an antigen binding domain that specifically binds to a peptide conjugate/MHC complex, wherein the peptide conjugate/MHC complex comprises:
(c) a peptide conjugate comprising the peptide covalently linked to a targeted covalent inhibitor or fragment thereof; and
(d) an MHC.

369. A pharmaceutical composition comprising a polypeptide of any one of claims 166-368, and a pharmaceutically acceptable carrier.

370. A method of treating cancer in a subject that has been treated with a free targeted covalent inhibitor, the method comprising administering to the subject the polypeptide of any one of claims 166-368 or the pharmaceutical composition of claim 238.

371. The method of claim 370, wherein the subject is refractory to a treatment with the free targeted covalent inhibitor.

372. A method of treating cancer in a subject, the method comprising administering to the subject the polypeptide of any one of claims 166-368 or the pharmaceutical composition of claim 369 after or simultaneously with administration of a small molecule drug.

373. A method of treating cancer in a subject, the method comprising
administering to the subject the targeted covalent inhibitor, and
administering to the subject the polypeptide of any one of claims 166-368 or the pharmaceutical composition of claim 369.

374. The method of claim 373, wherein the polypeptide of any one of claims 166-368 or the pharmaceutical composition of claim 369 is administered after administration of the targeted covalent inhibitor or simultaneously with the targeted covalent inhibitor.

375. A method of identifying a T-cell receptor (TCR) that recognizes the peptide conjugate/MHC complex of any one of claims 167-374, the method comprising:
(a) contacting a plurality of candidate TCRs with the peptide conjugate/MHC complex, and
(b) identifying at least one TCR that binds to the peptide conjugate/MHC complex.

376. A method of identifying a T-cell receptor (TCR) that recognizes the peptide conjugate/MHC complex of any one of claims 167-374, the method comprising:
(a) culturing T cells and antigen presenting cells (APCs) treated with the targeted covalent inhibitor, thereby generating peptide conjugate/MHC complex specific T cells with a T cell receptor (TCR) that binds to the peptide conjugate/MHC complex; and
(b) identifying at least one TCR from the peptide conjugate/MHC complex specific T cells that binds to the peptide conjugate/MHC complex.

377. The method of claim 375 or 376, wherein identifying in (b) comprises selecting or isolating the at least one TCR.

378. The method of claim 375 or 377, wherein the plurality of candidate TCRs is a plurality of soluble TCRs or a plurality of TCRs expressed on cell surface of a plurality of cells.

379. The method of claim 378, wherein the plurality of candidate TCRs is the plurality of TCRs expressed on cell surface of the plurality of cells, and identifying in (b) comprises isolating or selecting a cell comprising the at least one TCR based on an activation marker of the cell.

380. The method of claim 379, wherein the activation marker is a T cell activation marker.

381. The method of claim 380, wherein the T cell activation marker is CD26, CD27, CD28, CD30, CD154, CD40L, CD134, CD25, CD44, CD69, CD137, or KLRG1.

382. A T-cell receptor (TCR) comprising the at least one TCR identified in (b) of any one of claims 375-381.

383. The TCR of claim 382, wherein the TCR is a soluble TCR.

384. The TCR of claim 382 or 383, wherein the TCR is a bispecific TCR.

385. A method of treating cancer, the method comprising prophylactically administering a peptide conjugate to a subject in need thereof prior to the subject developing a cancer, or prior to the subject being administered a drug, or both, wherein the peptide conjugate comprises the peptide covalently linked to a targeted covalent inhibitor or fragment thereof.
